SEQUENCE LISTING (24 sequences)

SEQ ID NO: 1 - DNA, 444 bp, Artificial Sequence
  HBV truncated core antigen gene
gacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatcgaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggattcgcactccccctgcttatcgaccacccaacgcaccaattctgtccaccctgcccgagaccacagtggtc

SEQ ID NO: 2 - protein, 148 aa, Artificial Sequence
  HBV truncated core antigen
DIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVV

SEQ ID NO: 3 - DNA, 447 bp, Artificial Sequence
  HBV truncated core antigen gene
atggacatcgacccttacaaggagttcggcgccagcgtggaactgctgtcttttctgcccagtgatttctttccttccattcgagacctgctggataccgcctctgctctgtatcgggaagccctggagagcccagaacactgctccccacaccataccgctctgcgacaggcaatcctgtgctggggggagctgatgaacctggccacatgggtgggatccaatctggaggaccccgcttcacgggaactggtggtcagctacgtgaacgtcaatatgggcctgaaaatccgccagctgctgtggttccatattagctgcctgacttttggacgagagaccgtgctggaatacctggtgtccttcggcgtctggatccgcactccccctgcttatcgaccacccaacgcaccaattctgtccaccctgcccgagaccacagtggtc

SEQ ID NO: 4 - protein, 149 aa, Artificial Sequence
  HBV truncated core antigen
MDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVV

SEQ ID NO: 5 - DNA, 2529 bp, Artificial Sequence
  HBV pol antigen gene
atgcccctgtcttaccagcactttagaaagcttctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcacacgccacggggatgagtccttttgccagcagtctagtggcatcctgagcagatcccccgtggggccttgtctgcagtctcagctgcggaagagtagactgggactgcagccacagcagggacacctggcacgacggcagcagggaaggtctggcagtatccgggctagagtgcatcccacaactagaaggcctttcggcgtcgagccatcaggaagcggccacaccacaaacaccgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctcca

SEQ ID NO: 6 - DNA, 2529 bp, Artificial Sequence
  HBV pol antigen gene
atgcccctgtcttaccagcactttagaaagctgctgctgctggacgatgaagccgggcctctggaggaagagctgccaaggctggcagacgaggggctgaaccggagagtggccgaagatctgaatctgggaaacctgaacgtgagcatcccttggactcataaagtcggcaacttcaccgggctgtacagctccacagtgcctgtcttcaatccagagtggcagacaccatcctttcccaacattcacctgcaggaggacatcattaatagatgcgaacagttcgtgggacctctgacagtcaacgaaaagaggcgcctgaaactgatcatgcctgccaggttttacccaaatgtgactaagtatctgccactggataagggcatcaagccttactatccagagcacctggtgaaccattacttccagactagacactatctgcataccctgtggaaggccggaatcctgtacaaacgagaaactacccggagtgcttcattttgtggctccccatattcttgggaacaggagctgcagcatggcaggctggtgttccagaccagcacacgccacggggatgagtccttttgccagcagtctagtggcatcctgagcagatcccccgtggggccttgtctgcagtctcagctgcggaagagtagactgggactgcagccacagcagggacacctggcacgacggcagcagggaaggtctggcagtatccgggctagagtgcatcccacaactagaaggcctttcggcgtcgagccatcaggaagcggccacaccacaaacaccgcatcaagctcctctagttgcctgcatcagtcagccgtgagaaaggccgcttacagccacctgtccacatctaaaaggcactcaagctccgggcatgctgtggagctgcacaacatccctccaaattctgcacgcagtcagtcagaaggacccgtgttcagctgctggtggctgcagtttcggaactcaaagccttgcagcgactattgtctgagccatattgtgaatctgctggaggattggggcccttgtaccgagcacggggaacaccatatcaggattccacgaacaccagcacgagtgactggaggggtgttcctggtggacaagaacccccacaatactaccgagagccggctggtggtcgatttcagtcagttttcaagaggcaacacaagggtgtcatggcccaaattcgccgtccctaatctgcagagtctgactaacctgctgtctagtaatctgagctggctgtccctggacgtgtccgcagccttttaccacctgcctctgcatccagctgcaatgccccatctgctggtggggtcaagcggactgagtcgctacgtcgcccgactgtcctctaactcacgcatcattaatcaccagcatggcaccatgcagaacctgcacgatagctgttcccggaatctgtacgtgtctctgctgctgctgtataagacattcggcagaaaactgcacctgtacagccatcctatcattctggggtttaggaagatcccaatgggagtgggactgagccccttcctgctggcacagtttacctccgccatttgctctgtggtccgccgagccttcccacactgtctggctttttcctatatgaacaatgtggtcctgggcgccaaatccgtgcagcatctggagtctctgttcacagctgtcactaactttctgctgagcctggggatccacctgaacccaaataagactaaacgctgggggtacagcctgaatttcatgggatatgtgattggatcctgggggaccctgccacaggagcacatcgtgcagaagatcaaggaatgctttcggaagctgcccgtcaacagacctatcgactggaaagtgtgccagcggattgtcggactgctgggcttcgccgctccctttacccagtgcgggtacccagcactgatgcccctgtatgcctgtatccagtctaagcaggctttcacctttagtcctacatacaaggcattcctgtgcaaacagtacctgaacctgtatccagtggcaaggcagcgacctggactgtgccaggtctttgcaaatgccactcctaccggctgggggctggctatcggacatcagcgaatgcggggcacattcgtggcccccctgcctattcacactgctcagctgctggcagcctgctttgctagatctaggagtggagcaaagctgatcggcaccgacaatagtgtggtcctgtcaagaaaatacacatccttcccatggctgctgggatgtgctgcaaactggattctgaggggcaccagcttcgtgtacgtcccctcagccctgaatcctgctgacgatccatcccgcgggcgactgggactgtaccgacctctgctgagactgcccttcaggcctacaactggccggacatctctgtatgccgattcaccaagcgtgccctcacacctgcctgacagagtccactttgcttcacccctgcacgtcgcttggcggcctcca

SEQ ID NO: 7 - protein, 843 aa, Artificial Sequence
  HBV pol antigen
MPLSYQHFRKLLLLDDEAGPLEEELPRLADEGLNRRVAEDLNLGNLNVSIPWTHKVGNFTGLYSSTVPVFNPEWQTPSFPNIHLQEDIINRCEQFVGPLTVNEKRRLKLIMPARFYPNVTKYLPLDKGIKPYYPEHLVNHYFQTRHYLHTLWKAGILYKRETTRSASFCGSPYSWEQELQHGRLVFQTSTRHGDESFCQQSSGILSRSPVGPCLQSQLRKSRLGLQPQQGHLARRQQGRSGSIRARVHPTTRRPFGVEPSGSGHTTNTASSSSSCLHQSAVRKAAYSHLSTSKRHSSSGHAVELHNIPPNSARSQSEGPVFSCWWLQFRNSKPCSDYCLSHIVNLLEDWGPCTEHGEHHIRIPRTPARVTGGVFLVDKNPHNTTESRLVVDFSQFSRGNTRVSWPKFAVPNLQSLTNLLSSNLSWLSLDVSAAFYHLPLHPAAMPHLLVGSSGLSRYVARLSSNSRIINHQHGTMQNLHDSCSRNLYVSLLLLYKTFGRKLHLYSHPIILGFRKIPMGVGLSPFLLAQFTSAICSVVRRAFPHCLAFSYMNNVVLGAKSVQHLESLFTAVTNFLLSLGIHLNPNKTKRWGYSLNFMGYVIGSWGTLPQEHIVQKIKECFRKLPVNRPIDWKVCQRIVGLLGFAAPFTQCGYPALMPLYACIQSKQAFTFSPTYKAFLCKQYLNLYPVARQRPGLCQVFANATPTGWGLAIGHQRMRGTFVAPLPIHTAQLLAACFARSRSGAKLIGTDNSVVLSRKYTSFPWLLGCAANWILRGTSFVYVPSALNPADDPSRGRLGLYRPLLRLPFRPTTGRTSLYADSPSVPSHLPDRVHFASPLHVAWRPP

SEQ ID NO: 8 - DNA, 63 bp, Artificial Sequence
  Cystatin S signal peptide coding sequence
atggctcgacctctgtgtaccctgctactcctgatggctaccctggctggagctctggccagc

SEQ ID NO: 9 - protein, 21 aa, Artificial Sequence
  Cystatin S signal peptide sequence
MARPLCTLLLLMATLAGALAS

SEQ ID NO: 10 - DNA, 378 bp, Artificial Sequence
  triple enhancer regulatory sequence
ggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttctctcgttaacttaatgagacagatagaaactggtcttgtagaaacagagtagtcgcctgcttttctgccaggtgctgacttctctcccctgggcttttttctttttctcaggttgaaaagaagaagacgaagaagacgaagaagac

SEQ ID NO: 11 - DNA, 12 bp, Artificial Sequence
  linker coding sequence
gccggagctggc

SEQ ID NO: 12 - DNA, 248 bp, Artificial Sequence
  ApoAI gene fragment
ttggccgtgctcttcctgacgggtaggtgtcccctaacctagggagccaaccatcggggggccttctccctaaatccccgtggcccaccctcctgggcagaggcagcaggtttctcactggccccctctcccccacctccaagcttggcctttcggctcagatctcagcccacagctggcctgatctgggtctcccctcccaccctcagggagccaggctcggcatttcgtcgacaagcttagccacc

SEQ ID NO: 13 - DNA, 130 bp, Artificial Sequence
  SV40 polyadenylation signal sequence
aacttgtttattgcagcttataatggttacaaataaagcaatagcatcacaaatttcacaaataaagcatttttttcactgcattctagttgtggtttgtccaaactcatcaatgtatcttatcatgtct

SEQ ID NO: 14 - DNA, 81 bp, Artificial Sequence
  immunoglobulin secretion signal coding sequence
atggagttcggcctgtcttgggtctttctggtggcaatcctgaagggcgtgcagtgtgaagtgcagctgctggagtctgga

SEQ ID NO: 15 - protein, 27 aa, Artificial Sequence
  immunoglobulin secretion signal sequence
MEFGLSWVFLVAILKGVQCEVQLLESG

SEQ ID NO: 16 - protein, 996 aa, Artificial Sequence
  HBV core-pol fusion antigen sequence
MDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVAGAGMPLSYQHFRKLLLLDDEAGPLEEELPRLADEGLNRRVAEDLNLGNLNVSIPWTHKVGNFTGLYSSTVPVFNPEWQTPSFPNIHLQEDIINRCEQFVGPLTVNEKRRLKLIMPARFYPNVTKYLPLDKGIKPYYPEHLVNHYFQTRHYLHTLWKAGILYKRETTRSASFCGSPYSWEQELQHGRLVFQTSTRHGDESFCQQSSGILSRSPVGPCLQSQLRKSRLGLQPQQGHLARRQQGRSGSIRARVHPTTRRPFGVEPSGSGHTTNTASSSSSCLHQSAVRKAAYSHLSTSKRHSSSGHAVELHNIPPNSARSQSEGPVFSCWWLQFRNSKPCSDYCLSHIVNLLEDWGPCTEHGEHHIRIPRTPARVTGGVFLVDKNPHNTTESRLVVDFSQFSRGNTRVSWPKFAVPNLQSLTNLLSSNLSWLSLDVSAAFYHLPLHPAAMPHLLVGSSGLSRYVARLSSNSRIINHQHGTMQNLHDSCSRNLYVSLLLLYKTFGRKLHLYSHPIILGFRKIPMGVGLSPFLLAQFTSAICSVVRRAFPHCLAFSYMNNVVLGAKSVQHLESLFTAVTNFLLSLGIHLNPNKTKRWGYSLNFMGYVIGSWGTLPQEHIVQKIKECFRKLPVNRPIDWKVCQRIVGLLGFAAPFTQCGYPALMPLYACIQSKQAFTFSPTYKAFLCKQYLNLYPVARQRPGLCQVFANATPTGWGLAIGHQRMRGTFVAPLPIHTAQLLAACFARSRSGAKLIGTDNSVVLSRKYTSFPWLLGCAANWILRGTSFVYVPSALNPADDPSRGRLGLYRPLLRLPFRPTTGRTSLYADSPSVPSHLPDRVHFASPLHVAWRPP

SEQ ID NO: 17 - protein, 1023 aa, Artificial Sequence
  HBV core-pol fusion antigen sequence with Ig signal sequence
MEFGLSWVFLVAILKGVQCEVQLLESGMDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVAGAGMPLSYQHFRKLLLLDDEAGPLEEELPRLADEGLNRRVAEDLNLGNLNVSIPWTHKVGNFTGLYSSTVPVFNPEWQTPSFPNIHLQEDIINRCEQFVGPLTVNEKRRLKLIMPARFYPNVTKYLPLDKGIKPYYPEHLVNHYFQTRHYLHTLWKAGILYKRETTRSASFCGSPYSWEQELQHGRLVFQTSTRHGDESFCQQSSGILSRSPVGPCLQSQLRKSRLGLQPQQGHLARRQQGRSGSIRARVHPTTRRPFGVEPSGSGHTTNTASSSSSCLHQSAVRKAAYSHLSTSKRHSSSGHAVELHNIPPNSARSQSEGPVFSCWWLQFRNSKPCSDYCLSHIVNLLEDWGPCTEHGEHHIRIPRTPARVTGGVFLVDKNPHNTTESRLVVDFSQFSRGNTRVSWPKFAVPNLQSLTNLLSSNLSWLSLDVSAAFYHLPLHPAAMPHLLVGSSGLSRYVARLSSNSRIINHQHGTMQNLHDSCSRNLYVSLLLLYKTFGRKLHLYSHPIILGFRKIPMGVGLSPFLLAQFTSAICSVVRRAFPHCLAFSYMNNVVLGAKSVQHLESLFTAVTNFLLSLGIHLNPNKTKRWGYSLNFMGYVIGSWGTLPQEHIVQKIKECFRKLPVNRPIDWKVCQRIVGLLGFAAPFTQCGYPALMPLYACIQSKQAFTFSPTYKAFLCKQYLNLYPVARQRPGLCQVFANATPTGWGLAIGHQRMRGTFVAPLPIHTAQLLAACFARSRSGAKLIGTDNSVVLSRKYTSFPWLLGCAANWILRGTSFVYVPSALNPADDPSRGRLGLYRPLLRLPFRPTTGRTSLYADSPSVPSHLPDRVHFASPLHVAWRPP

SEQ ID NO: 18 - DNA, 584 bp, Artificial Sequence
  hCMV promoter
tgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagc

SEQ ID NO: 19 - DNA, 684 bp, Artificial Sequence
  hCMV promoter sequence
accgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgg

SEQ ID NO: 20 - DNA, 225 bp, Artificial Sequence
  bGH polyA signal
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 21 - DNA, 671 bp, Artificial Sequence
  pUC ORI
cccgtagaaaagatcaaaggatcttcttgagatcctttttttctgcgcgtaatctgctgcttgcaaacaaaaaaaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacat

SEQ ID NO: 22 - DNA, 795 bp, Artificial Sequence
  KanR coding sequence
atgattgagcaagatggtcttcacgctggctcgccagctgcgtgggtggaacgcctgtttggttatgattgggcgcagcagactattggatgttccgacgcggctgtatttcggctgtctgctcagggtcgccccgtgctgtttgtgaagacggatttgtctggcgcattaaatgagttacaggacgaggcggctcgtctgagttggttggccaccaccggcgtgccctgcgccgcagtgctggatgtcgtgacagaagcaggccgcgattggctccttctcggcgaagtgccgggccaggacctgctcagcagccacttggcaccggcagaaaaagtttctatcatggccgacgccatgcgtcgtcttcacactctcgatccggccacgtgcccctttgaccaccaggccaagcatcgtattgaacgtgcgcgtactcggatggaagcaggtttagtagaccaggacgatttggatgaggaacatcaaggcctggccccggctgaactgtttgcgcgcttaaaagcgtcgatgccagatggcgaagatttggtagtcacccatggagatgcgtgtttgccaaacatcatggttgaaaatggccgcttctcaggctttattgactgtgggcgcctgggtgttgccgaccgctatcaagatattgcgctcgcaactcgtgacatcgctgaagagctgggcggagaatgggctgaccgtttcctggtactgtatggcattgcagcgcccgattcccaacgcatcgcattttatcgtctgctggatgagtttttctaa

SEQ ID NO: 23 - protein, 264 aa, Artificial Sequence
  Codon optimized Kanr
MIEQDGLHAGSPAAWVERLFGYDWAQQTIGCSDAAVFRLSAQGRPVLFVKTDLSGALNELQDEAARLSWLATTGVPCAAVLDVVTEAGRDWLLLGEVPGQDLLSSHLAPAEKVSIMADAMRRLHTLDPATCPFDHQAKHRIERARTRMEAGLVDQDDLDEEHQGLAPAELFARLKASMPDGEDLVVTHGDACLPNIMVENGRFSGFIDCGRLGVADRYQDIALATRDIAEELGGEWADRFLVLYGIAAPDSQRIAFYRLLDEFF

SEQ ID NO: 24 - DNA, 99 bp, Artificial Sequence
  bla promoter
acccctatttgtttatttttctaaatacattcaaatatgtatccgctcatgagacaataaccctgataaatgcttcaataatattgaaaaaggaagagt

(19) United States
(12) Patent Application Publication
(10) Pub. No.: US 2022/0305115 A1
(43) Pub. Date: Sep. 29, 2022

(54) COMBINATION OF HEPATITIS B VIRUS (HBV) VACCINES AND PYRIDOPYRIMIDINE DERIVATIVES

(71) Applicant: JANSSEN SCIENCES IRELAND UNLIMITED COMPANY, Co Cork (IE)

(72) Inventors: Helen HORTON, Beerse (BE); Antony Chien-Hung CHEN, Beerse (BE)

(21) Appl. No.: 17/596,210

(22) PCT Filed: Jun. 18, 2020
(86) PCT No.: PCT/IB2020/055743

§ 371 Date: Dec. 6, 2021

Related U.S. Application Data
(60) Provisional application No. 62/863,206, filed on Jun. 18, 2019.

(51) Int. Cl. A61K 39/29; A61K 31/519; A61P 31/20; A61K 31/55

(52) U.S. Cl. CPC A61K 39/292; A61K 31/519; A61P 31/20; A61K 31/55; A61K 2039/525

(57) ABSTRACT

Therapeutic combinations of hepatitis B virus (HBV) vaccines and a pyridopyrimidine derivative are described. Methods of inducing an immune response against HBV or treating an HBV-induced disease, particularly in individuals having chronic HBV infection, using the disclosed therapeutic combinations are also described. The invention provides therapeutic combinations or compositions and methods for inducing an immune response against hepatitis B viruses (HBV) infection.

### CROSS REFERENCE TO RELATED APPLICATION

This application claims priority to U.S. Provisional Application No. 62/863,206 filed on Jun. 18, 2019, the disclosure of which is incorporated herein by reference in its entirety.

### REFERENCE TO SEQUENCE LISTING SUBMITTED ELECTRONICALLY

This application contains a sequence listing, which is submitted electronically via EFS-Web as an ASCII formatted sequence listing with a file name “065814_20WO1_Sequence_Listing” and a creation date of Jun. 17, 2020 and having a size of 46 kb. The sequence listing submitted via EFS-Web is part of the specification and is herein incorporated by reference in its entirety.

### BACKGROUND OF THE INVENTION

Hepatitis B virus (HBV) is a small 3.2-kb hepatotropic DNA virus that encodes four open reading frames and seven proteins. Approximately 240 million people have chronic hepatitis B infection (chronic HBV), characterized by persistent virus and subvirus particles in the blood for more than 6 months (Cohen et al. J. Viral Hepat. (2011) 18(6), 377-83). Persistent HBV infection leads to T-cell exhaustion in circulating and intrahepatic HBV-specific CD4+ and CD8+ T-cells through chronic stimulation of HBV-specific T-cell receptors with viral peptides and circulating antigens. As a result, T-cell polyfunctionality is decreased (i.e., decreased levels of IL-2, tumor necrosis factor (TNF)-α, IFN-γ, and lack of proliferation).

A safe and effective prophylactic vaccine against HBV infection has been available since the 1980s and is the mainstay of hepatitis B prevention (World Health Organization, Hepatitis B: Fact sheet No. 204 [Internet] 2015 March). The World Health Organization recommends vaccination of all infants, and, in countries where there is low or intermediate hepatitis B endemicity, vaccination of all children and adolescents (<18 years of age), and of people of certain at risk population categories. Due to vaccination, worldwide infection rates have dropped dramatically. However, prophylactic vaccines do not cure established HBV infection.

Chronic HBV is currently treated with IFN-α and nucleoside or nucleotide analogs, but there is no ultimate cure due to the persistence in infected hepatocytes of an intracellular viral replication intermediate called covalently closed circular DNA (cccDNA), which plays a fundamental role as a template for viral RNAs, and thus new virions. It is thought that induced virus-specific T-cell and B-cell responses can effectively eliminate cccDNA-carrying hepatocytes. Current therapies targeting the HBV polymerase suppress viremia, but offer limited effect on cccDNA that resides in the nucleus and related production of circulating antigen. The most rigorous form of a cure may be elimination of HBV cccDNA from the organism, which has neither been observed as a naturally occurring outcome nor as a result of any therapeutic intervention. However, loss of HBV surface antigens (HBsAg) is a clinically credible equivalent of a cure, since disease relapse can occur only in cases of severe immunosuppression, which can then be prevented by prophylactic treatment. Thus, at least from a clinical standpoint, loss of HBsAg is associated with the most stringent form of immune reconstitution against HBV.

For example, immune modulation with pegylated interferon (pegIFN)-α has proven better in comparison to nucleoside or nucleotide therapy in terms of sustained off-treatment response with a finite treatment course. Besides a direct antiviral effect, IFN-α is reported to exert epigenetic suppression of cccDNA in cell culture and humanized mice, which leads to reduction of virion productivity and transcripts (Belloni et al. J. Clin. Invest. (2012) 122(2), 529-537). However, this therapy is still fraught with side-effects and overall responses are rather low, in part because IFN-α has only poor modulatory influences on HBV-specific T-cells. In particular, cure rates are low (<10%) and toxicity is high. Likewise, direct acting HBV antivirals, namely the HBV polymerase inhibitors entecavir and tenofovir, are effective as monotherapy in inducing viral suppression with a high genetic barrier to emergence of drug resistant mutants and consecutive prevention of liver disease progression. However, cure of chronic hepatitis B, defined by HBsAg loss or seroconversion, is rarely achieved with such HBV polymerase inhibitors. Therefore, these antivirals in theory need to be administered indefinitely to prevent reoccurrence of liver disease, similar to antiretroviral therapy for human immunodeficiency virus (HIV).

Therapeutic vaccination has the potential to eliminate HBV from chronically infected patients (Michel et al. J. Hepatol. (2011) 54(6), 1286-1296). Many strategies have been explored, but to date therapeutic vaccination has not proven successful.

### BRIEF SUMMARY OF THE INVENTION

Accordingly, there is an unmet medical need in the treatment of hepatitis B virus (HBV), particularly chronic HBV, for a finite well-tolerated treatment with a higher cure rate. The invention satisfies this need by providing therapeutic combinations or compositions and methods for inducing an immune response against hepatitis B viruses (HBV) infection. The immunogenic compositions/combinations and methods of the invention can be used to provide therapeutic immunity to a subject, such as a subject having chronic HBV infection.

In a general aspect, the application relates to therapeutic combinations or compositions comprising one or more HBV antigens, or one or more polynucleotides encoding the HBV antigens, and a pyridopyrimidine derivative, for use in treating an HBV infection in a subject in need thereof.

In one embodiment, the therapeutic composition comprises:

i) at least one of:
a) a truncated HBV core antigen consisting of an amino acid sequence that is at least 95%, such as at least 95%, 96%, 97%, 98%, 99% or 100%, identical to SEQ ID NO: 2,
b) a first non-naturally occurring nucleic acid molecule comprising a first polynucleotide sequence encoding the truncated HBV core antigen;
c) an HBV polymerase antigen having an amino acid sequence that is at least 90%, such as at least 90%, 91%, 92%, 93%, 94%, 95%, 96%, 97%, 98%, 99% or 100%, identical to SEQ ID NO: 7, wherein the HBV polymerase antigen does not have reverse transcriptase activity and RNase H activity, and
d) a second non-naturally occurring nucleic acid molecule comprising a second polynucleotide sequence encoding the HBV polymerase antigen; and
   ii) a benzazepine carboxamide compound of formula (K):

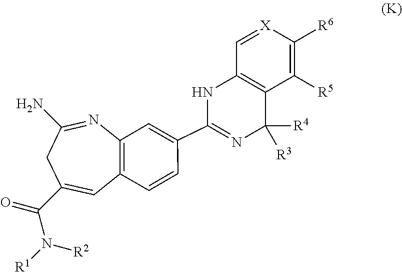

or a pharmaceutically acceptable salt thereof,
wherein
R¹ is C₃₋₇-alkyl;
R² is C₃₋₇-alkyl or C₃₋₇-cycloalkyl-C₁₋₇-alkyl;
R³ is hydrogen or C₁₋₇-alkyl;
R⁴ is hydrogen or C₁₋₇-alkyl;
R⁵ is selected from the group consisting of hydrogen, halogen, C₁₋₇-alkyl and C₁₋₇-alkoxy;
R⁶ is selected from the group consisting of hydrogen, halogen, C₁₋₇-alkyl and C₁₋₇-alkoxy; and
X is N or CR⁷, wherein R⁷ is selected from the group consisting of hydrogen, halogen, C₁₋₇-alkyl and C₁₋₇-alkoxy.

In another embodiment, the therapeutic composition comprises:

i) at least one of:
a) a truncated HBV core antigen consisting of an amino acid sequence that is at least 95%, such as at least 95%, 96%, 97%, 98%, 99% or 100%, identical to SEQ ID NO: 2,
b) a first non-naturally occurring nucleic acid molecule comprising a first polynucleotide sequence encoding the truncated HBV core antigen;
c) an HBV polymerase antigen having an amino acid sequence that is at least 90%, such as at least 90%, 91%, 92%, 93%, 94%, 95%, 96%, 97%, 98%, 99% or 100%, identical to SEQ ID NO: 7, wherein the HBV polymerase antigen does not have reverse transcriptase activity and RNase H activity, and
d) a second non-naturally occurring nucleic acid molecule comprising a second polynucleotide sequence encoding the HBV polymerase antigen; and
   ii) a pyridopyrimidine compound of formula (J):

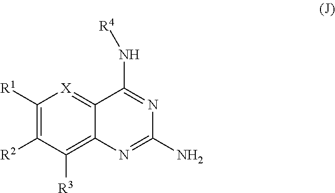

or a pharmaceutically acceptable salt thereof,
wherein

### X is N or CR¹⁰;

R¹ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups;
R² is selected from the group consisting of hydrogen, halogen, C₁₋₆alkyl, CN, —NR^{a}R^{b}, —S(O)₁₋₂R^{a} and OR^{a}, wherein C₁₋₆alkyl is optionally substituted with 1 to 5 R²⁰ groups:
R³ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups;
R⁴ is C₁₋₁₂ alkyl which is optionally substituted with 1 to 5 substituents independently selected from halogen, —OR^{a}, —NR^{a}R^{b},
CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O) OR^{b}, —SR^{a}, —S(O)₁₋₂R^{a}, —S(O)₂NR^{a}R^{b}, —NR^{a}S(O)₂R^{b}, C₁₋₆haloalkyl, C₃₋₆cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur;
wherein each C₃₋₆cycloalkyl, 3 to 6 membered heterocyclyl, C₆₋₁₀ aryl, and 5 to 10 membered heteroaryl is optionally substituted with 1 to 5 R²¹ groups;
R¹⁰ is selected from hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups
each R²⁰ is independently selected from the group consisting of halogen, C₁₋₆haloalkyl, CN, —NR^{a}R^{b}, S(O)₁₋₂R^{a}, and OR^{a};
each R²¹ is independently selected from the group consisting of halogen, C₁₋₆alkyl, C₁₋₆haloalkyl, CN, —NR^{a}R^{b}, S(O)₁₋₂R^{a}, and OR^{a}; and
each R^{a} and R^{b} are independently selected from the group consisting of hydrogen and C₁₋₆alkyl; wherein each C₁₋₆alkyl is optionally substituted with 1 to 5 substituents independently selected from halogen, hydroxyl, amino, 5 to 10 membered heteroaryl wherein the 5 to 10 membered heteroaryl has 1 to 3 heteroatoms selected from oxygen, nitrogen, and sulfur, and C₁₋₆haloalkyl; provided that when X is N, R¹ is Cl, R² is H and R³ is H then R⁴ is not CH₂CH₂OMe or CH₂CH₂SO₂Me.

In another embodiment, the therapeutic composition comprises:

i) at least one of:
a) a truncated HBV core antigen consisting of an amino acid sequence that is at least 95%, such as at least 95%, 96%, 97%, 98%, 99% or 100%, identical to SEQ ID NO: 2,
b) a first non-naturally occurring nucleic acid molecule comprising a first polynucleotide sequence encoding the truncated HBV core antigen;
c) an HBV polymerase antigen having an amino acid sequence that is at least 90%, such as at least 90%, 91%, 92%, 93%, 94%, 95%, 96%, 97%, 98%, 99% or 100%, identical to SEQ ID NO: 7, wherein the HBV polymerase antigen does not have reverse transcriptase activity and RNase H activity, and
d) a second non-naturally occurring nucleic acid molecule comprising a second polynucleotide sequence encoding the HBV polymerase antigen; and
   ii) a pyridopyrimidine compound of formula (I):

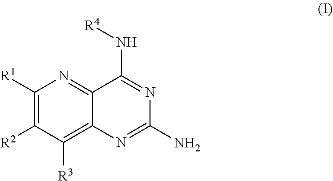

or a pharmaceutically acceptable salt thereof, wherein:
R¹ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups;
R² is selected from the group consisting of hydrogen, halogen, C₁₋₆alkyl, CN, —NR^{a}R^{b}, —S(O)₁₋₂R^{a} and OR^{a}, wherein C₁₋₆alkyl optionally substituted with 1 to 5 R²⁰ groups;
R³ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups;
R⁴ is C₁₋₁₂ alkyl which is optionally substituted with 1 to 5 substituents independently selected from halogen, —OR^{a}, —NR^{a}R^{b},
CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆ haloalkyl, C₃₋₆cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur;
wherein each C₃₋₆cycloalkyl, 3 to 6 membered heterocyclyl, C₆₋₁₀ aryl, and 5 to 10 membered heteroaryl is optionally substituted with 1 to 5 R²¹ groups;
each R²⁰ is independently selected from the group consisting of halogen, C₁₋₆haloalkyl, CN, —NR^{a}R^{b}, S(O)₁₋₂R^{a}, and OR^{a};
each R²¹ is independently selected from the group consisting of halogen, C₁₋₆alkyl, C₁₋₆haloalkyl, CN, —NR^{a}R^{b}, S(O)₁₋₂R^{a}, and OR^{a}; and
each R^{a} and R^{b} are independently selected from the group consisting of hydrogen and C₁₋₆alkyl, wherein each C₁₋₆alkyl is optionally substituted with 1 to 5 substituents independently selected from halogen, hydroxyl, amino, 5 to 10 membered heteroaryl wherein the 5 to 10 membered heteroaryl has 1 to 3 heteroatoms selected from oxygen, nitrogen, and sulfur, and C₁₋₆haloalkyl; provided that when R¹ is Cl, R² is H and R³ is H then R⁴ is not CH₂CH₂OMe or CH₂CH₂SO₂Me.

In one embodiment, the truncated HBV core antigen consists of the amino acid sequence of SEQ ID NO: 2 or SEQ ID NO: 4, and the HBV polymerase antigen comprises the amino acid sequence of SEQ ID NO: 7.

In one embodiment, the therapeutic combination comprises at least one of the HBV polymerase antigen and the truncated HBV core antigen. In certain embodiments, the therapeutic combination comprises the HBV polymerase antigen and the truncated HBV core antigen.

In one embodiment, the therapeutic combination comprises at least one of the first non-naturally occurring nucleic acid molecule comprising the first polynucleotide sequence encoding the truncated HBV core antigen, and the second non-naturally occurring nucleic acid molecule comprising the second polynucleotide sequence encoding the HBV polymerase antigen. In certain embodiments, the first non-naturally occurring nucleic acid molecule further comprises a polynucleotide sequence encoding a signal sequence operably linked to the N-terminus of the truncated HBV core antigen, and the second non-naturally occurring nucleic acid molecule further comprises a polynucleotide sequence encoding a signal sequence operably linked to the N-terminus of the HBV polymerase antigen, preferably, the signal sequence independently comprises the amino acid sequence of SEQ ID NO: 9 or SEQ ID NO: 15, more preferably, the signal sequence is encoded by the polynucleotide sequence of SEQ ID NO: 8 or SEQ ID NO: 14, respectively.

In certain embodiments, the first polynucleotide sequence comprises the polynucleotide sequence having at least 90%, such as at least 90%, 91%, 92%, 93%, 94%, 95%, 96%, 97%, 98%, 99% or 100%, sequence identity to SEQ ID NO: 1 or SEQ ID NO: 3.

In certain embodiments, the second polynucleotide sequence comprises a polynucleotide sequence having at least 90%, such as at least 90%, 91%, 92%, 93%, 94%, 95%, 96%, 97%, 98%, 99% or 100%, sequence identity to SEQ ID NO: 5 or SEQ ID NO: 6.

In an embodiment, a therapeutic combination comprises:
a) a first non-naturally occurring nucleic acid molecule comprising a first polynucleotide sequence encoding a truncated HBV core antigen consisting of an amino acid sequence that is at least 95%, such as at least 95%, 96%, 97%, 98%, 99% or 100%, identical to SEQ ID NO: 2;
b) a second non-naturally occurring nucleic acid molecule comprising a second polynucleotide sequence encoding an HBV polymerase antigen having an amino acid sequence that is at least 90%, such as at least 90%, 91%, 92%, 93%, 94%, 95%, 96%, 97%, 98%, 99% or 100%, identical to SEQ ID NO: 7, wherein the HBV polymerase antigen does not have reverse transcriptase activity and RNase H activity; and
c) a compound of formula (K)

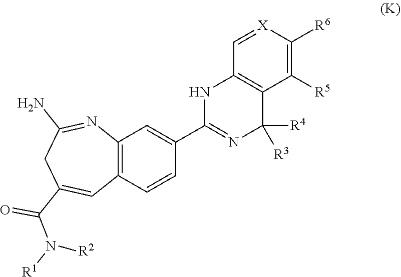

or a pharmaceutically acceptable salt thereof;
wherein
R¹ is C₃₋₇-alkyl;
R² is C₃₋₇-alkyl or C₃₋₇-cycloalkyl-C₁₋₇-alkyl;
R³ is hydrogen or C₁₋₇-alkyl;
R⁴ is hydrogen or C₁₋₇-alkyl;
R⁵ is selected from the group consisting of hydrogen, halogen, C₁₋₇-alkyl and C₁₋₇-alkoxy;
R⁶ is selected from the group consisting of hydrogen, halogen, C₁₋₇-alkyl and C₁₋₇-alkoxy;
X is N or CR⁷, wherein R⁷ is selected from the group consisting of hydrogen, halogen, C₁₋₇-alkyl and C₁₋₇-alkoxy.

In another embodiment, a therapeutic combination comprises:
a) a first non-naturally occurring nucleic acid molecule comprising a first polynucleotide sequence encoding a truncated HBV core antigen consisting of an amino acid sequence that is at least 95%, such as at least 95%, 96%, 97%, 98%, 99% or 100%, identical to SEQ ID NO: 2;
b) a second non-naturally occurring nucleic acid molecule comprising a second polynucleotide sequence encoding an HBV polymerase antigen having an amino acid sequence that is at least 90%, such as at least 90%, 91%, 92%, 93%, 94%, 95%, 96%, 97%, 98%, 99% or 100%, identical to SEQ ID NO: 7, wherein the HBV polymerase antigen does not have reverse transcriptase activity and RNase H activity; and
c) a compound of formula (J)

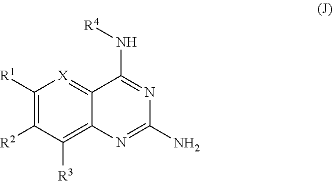

or a pharmaceutically acceptable salt thereof, wherein:

### X is N or CR¹⁰;

R¹ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups;
R² is selected from the group consisting of hydrogen, halogen, C₁₋₆alkyl, CN, —NR^{a}R^{b}, —S(O)₁₋₂R^{a} and OR^{a}, wherein C₁₋₆alkyl is optionally substituted with 1 to 5 R²⁰ groups;
R³ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups;
R⁴ is C₁₋₁₂ alkyl which is optionally substituted with 1 to 5 substituents independently selected from halogen, —OR^{a}, —NR^{a}R^{b},
CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O) OR^{b}, —SR^{a}, —S(O)₁₋₂R^{a}, —S(O)₂NR^{a}R^{b}, —NR^{a}S(O)₂R^{b}, C₁₋₆haloalkyl, C₃₋₆cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur;
wherein each C₃₋₆cycloalkyl, 3 to 6 membered heterocyclyl, C₆₋₁₀ aryl, and 5 to 10 membered heteroaryl is optionally substituted with 1 to 5 R²¹ groups;
R¹⁰ is selected from hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups
each R²⁰ is independently selected from the group consisting of halogen, C₁₋₆haloalkyl, CN, —NR^{a}R^{b}, S(O)₁₋₂R^{a}, and OR^{a};
each R²¹ is independently selected from the group consisting of halogen, C₁₋₆alkyl, C₁₋₆haloalkyl, CN, —NR^{a}R^{b}, S(O)₁₋₂R^{a}, and OR^{a}; and
each R^{a} and R^{b} are independently selected from the group consisting of hydrogen and C₀₋₆alkyl; wherein each C₁₋₆alkyl is optionally substituted with 1 to 5 substituents independently selected from halogen, hydroxyl, amino, 5 to 10 membered heteroaryl wherein the 5 to 10 membered heteroaryl has 1 to 3 heteroatoms selected from oxygen, nitrogen, and sulfur, and C₁₋₆haloalkyl; provided that when X is N, R¹ is Cl, R² is H and R³ is H then R⁴ is not CH₂CH₂OMe or CH₂CH₂SO₂Me.

In another embodiment, a therapeutic combination comprises:
a) a first non-naturally occurring nucleic acid molecule comprising a first polynucleotide sequence encoding a truncated HBV core antigen consisting of an amino acid sequence that is at least 95%, such as at least 95%, 96%, 97%, 98%, 99% or 100%, identical to SEQ ID NO: 2;
b) a second non-naturally occurring nucleic acid molecule comprising a second polynucleotide sequence encoding an HBV polymerase antigen having an amino acid sequence that is at least 90%, such as at least 90%, 91%, 92%, 93%, 94%, 95%, 96%, 97%, 98%, 99% or 100%, identical to SEQ ID NO: 7, wherein the HBV polymerase antigen does not have reverse transcriptase activity and RNase H activity; and
c) a compound of formula (I)

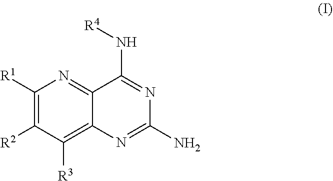

or a pharmaceutically acceptable salt thereof, wherein:
R¹ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups;
R² is selected from the group consisting of hydrogen, halogen, C₁₋₆alkyl, CN, —NR^{a}R^{b}, —S(O)₁₋₂R^{a} and OR^{a}, wherein C₁₋₆alkyl optionally substituted with 1 to 5 R²⁰ groups;
R³ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups;
R⁴ is C₁₋₁₂ alkyl which is optionally substituted with 1 to 5 substituents independently selected from halogen, —OR^{a}, —NR^{a}R^{b},
CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆haloalkyl, C₃₋₆cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur;
wherein each C₃₋₆cycloalkyl, 3 to 6 membered heterocyclyl, C₆₋₁₀ aryl, and 5 to 10 membered heteroaryl is optionally substituted with 1 to 5 R²¹ groups;
each R²⁰ is independently selected from the group consisting of halogen, C₁₋₆haloalkyl, CN, —NR^{a}R^{b}, S(O)₁₋₂R^{a}, and OR^{a};
each R²¹ is independently selected from the group consisting of halogen, C₁₋₆alkyl, C₁₋₆haloalkyl, CN, —NR^{a}R^{b}, S(O)₁₋₂R^{a}, and OR^{a}; and
each R^{a} and R^{b} are independently selected from the group consisting of hydrogen and C₁₋₆alkyl, wherein each C₁₋₆alkyl is optionally substituted with 1 to 5 substituents independently selected from halogen, hydroxyl, amino, 5 to 10 membered heteroaryl wherein the 5 to 10 membered heteroaryl has 1 to 3 heteroatoms selected from oxygen, nitrogen, and sulfur, and C₁₋₆haloalkyl; provided that when R¹ is Cl, R² is H and R³ is H then R⁴ is not CH₂CH₂OMe or CH₂CH₂SO₂Me.

Preferably, the therapeutic combination comprises a) a first non-naturally occurring nucleic acid molecule comprising a first polynucleotide sequence encoding an truncated HBV core antigen consisting of the amino acid sequence of SEQ ID NO: 2 or SEQ ID NO: 4; b) a second non-naturally occurring nucleic acid molecule comprising a second polynucleotide sequence encoding an HBV polymerase antigen having the amino acid sequence of SEQ ID NO: 7, and (c) a compound of formula (K), of formula (J), or of formula (I).

Preferably, the therapeutic combination comprises a first non-naturally occurring nucleic acid molecule comprising a polynucleotide sequence having at least 90%, such as at least 90%, 91%, 92%, 93%, 94%, 95%, 96%, 97%, 98%, 99% or 100%, sequence identity to SEQ ID NO: 1 or SEQ ID NO: 3, and a second non-naturally occurring nucleic acid molecule comprising the polynucleotide sequence having at least 90%, such as at least 90%, 91%, 92%, 93%, 94%, 95%, 96%, 97%, 98%, 99% or 100%, sequence identity to SEQ ID NO: 5 or SEQ ID NO: 6.

More preferably, the therapeutic combination comprises a) a first non-naturally occurring nucleic acid molecule comprising a first polynucleotide sequence of SEQ ID NO: 1 or SEQ ID NO: 3; b) a second non-naturally occurring nucleic acid molecule comprising a second polynucleotide sequence of SEQ ID NO: 5 or 6; and c) a compound of formula (K), of formula (J), or of formula (I).

In an embodiment, each of the first and the second non-naturally occurring nucleic acid molecules is a DNA molecule, preferably the DNA molecule is present on a plasmid or a viral vector.

In another embodiment, each of the first and the second non-naturally occurring nucleic acid molecules is an RNA molecule, preferably an mRNA or a self-replicating RNA molecule.

In some embodiments, each of the first and the second non-naturally occurring nucleic acid molecules is independently formulated with a lipid nanoparticle (LNP).

In another general aspect, the application relates to a kit comprising a therapeutic combination of the application.

The application also relates to a therapeutic combination or kit of the application for use in inducing an immune response against hepatitis B virus (HBV); and use of a therapeutic combination, composition or kit of the application in the manufacture of a medicament for inducing an immune response against hepatitis B virus (HBV). The use can further comprise a combination with another immunogenic or therapeutic agent, preferably another HBV antigen or another HBV therapy. Preferably, the subject has chronic HBV infection.

The application further relates to a therapeutic combination or kit of the application for use in treating an HBV-induced disease in a subject in need thereof; and use of therapeutic combination or kit of the application in the manufacture of a medicament for treating an HBV-induced disease in a subject in need thereof. The use can further comprise a combination with another therapeutic agent, preferably another anti-HBV antigen. Preferably, the subject has chronic HBV infection, and the HBV-induced disease is selected from the group consisting of advanced fibrosis, cirrhosis, and hepatocellular carcinoma (HCC).

The application also relates to a method of inducing an immune response against an HBV or a method of treating an HBV infection or an HBV-induced disease, comprising administering to a subject in need thereof a therapeutic combination according to embodiments of the invention.

Other aspects, features and advantages of the invention will be apparent from the following disclosure, including the detailed description of the invention and its preferred embodiments and the appended claims.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIG. 1A and FIG. 1B show schematic representations of DNA plasmids according to embodiments of the application; FIG. 1A shows a DNA plasmid encoding an HBV core antigen according to an embodiment of the application; FIG. 1B shows a DNA plasmid encoding an HBV polymerase (pol) antigen according to an embodiment of the application; the HBV core and pol antigens are expressed under control of a CMV promoter with an N-terminal cystatin S signal peptide that is cleaved from the expressed antigen upon secretion from the cell; transcriptional regulatory elements of the plasmid include an enhancer sequence located between the CMV promoter and the polynucleotide sequence encoding the HBV antigen and a bGH polyadenylation sequence located downstream of the polynucleotide sequence encoding the HBV antigen; a second expression cassette is included in the plasmid in reverse orientation including a kanamycin resistance gene under control of an Ampr (bla) promoter; an origin of replication (pUC) is also included in reverse orientation.

FIG. 2A and FIG. 2B. show the schematic representations of the expression cassettes in adenoviral vectors according to embodiments of the application; FIG. 2A shows the expression cassette for a truncated HBV core antigen, which contains a CMV promoter, an intron (a fragment derived from the human ApoAI gene—GenBank accession X01038 base pairs 295-523, harboring the ApoAI second intron), a human immunoglobulin secretion signal, followed by a coding sequence for a truncated HBV core antigen and a SV40 polyadenylation signal; FIG. 2B shows the expression cassette for a fusion protein of a truncated HBV core antigen operably linked to an HBV polymerase antigen, which is otherwise identical to the expression cassette for the truncated HBV core antigen except the HBV antigen.

FIG. 3 shows ELISPOT responses of Balb/c mice immunized with different DNA plasmids expressing HBV core antigen or HBV pol antigen, as described in Example 3; peptide pools used to stimulate splenocytes isolated from the various vaccinated animal groups are indicated in gray scale; the number of responsive T-cells are indicated on the y-axis expressed as spot forming cells (SFC) per 10⁶ splenocytes;

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification. All patents, published patent applications and publications cited herein are incorporated by reference as if set forth fully herein. International Application PCT/US2016/020499, filed Mar. 2, 2016 (published as International Application Publication WO 2016/141092 on Sep. 9, 2016), U.S. patent application Ser. No. 15/059,070, filed Mar. 2, 2016 (published as U.S. Patent Application Publication 2016-0289229 A1 on Oct. 6, 2016), International Application No. PCT/EP2017/064107, filed Jun. 9, 2017 (published as International Application Publication No. WO2017/216054, filed Dec. 21, 2017), and U.S. patent application Ser. No. 16/213,308, filed Dec. 7, 2018 (published as U.S. Patent Application Publication 2019-0135788 on May 9, 2019) are hereby incorporated by reference in their entireties.

It must be noted that as used herein and in the appended claims, the singular forms “a,” “an,” and “the” include plural reference unless the context clearly dictates otherwise.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the application can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Unless otherwise stated, any numerical value, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1 mg/mL to 10 mg/mL includes 0.9 mg/mL to 11 mg/mL. As used herein, the use of a numerical range expressly includes all possible subranges, all individual numerical values within that range, including integers within such ranges and fractions of the values unless the context clearly indicates otherwise.

The phrases “percent (%) sequence identity” or “% identity” or “% identical to” when used with reference to an amino acid sequence describe the number of matches (“hits”) of identical amino acids of two or more aligned amino acid sequences as compared to the number of amino acid residues making up the overall length of the amino acid sequences. In other terms, using an alignment, for two or more sequences the percentage of amino acid residues that are the same (e.g. 90%, 91%, 92%, 93%, 94%, 95%, 97%, 98%, 99%, or 100% identity over the full-length of the amino acid sequences) may be determined, when the sequences are compared and aligned for maximum correspondence as measured using a sequence comparison algorithm as known in the art, or when manually aligned and visually inspected. The sequences which are compared to determine sequence identity may thus differ by substitution(s), addition(s) or deletion(s) of amino acids. Suitable programs for aligning protein sequences are known to the skilled person. The percentage sequence identity of protein sequences can, for example, be determined with programs such as CLUSTALW, Clustal Omega, FASTA or BLAST, e.g. using the NCBI BLAST algorithm (Altschul S F, et al (1997), Nucleic Acids Res. 25:3389-3402).

As used herein, the terms and phrases “in combination,” “in combination with,” “co-delivery,” and “administered together with” in the context of the administration of two or more therapies or components to a subject refers to simultaneous administration or subsequent administration of two or more therapies or components, such as two vectors, e.g., DNA plasmids, peptides, or a therapeutic combination and an adjuvant. “Simultaneous administration” can be administration of the two or more therapies or components at least within the same day. When two components are “administered together with” or “administered in combination with,” they can be administered in separate compositions sequentially within a short time period, such as 24, 20, 16, 12, 8 or 4 hours, or within 1 hour, or they can be administered in a single composition at the same time. “Subsequent administration” can be administration of the two or more therapies or components in the same day or on separate days. The use of the term “in combination with” does not restrict the order in which therapies or components are administered to a subject. For example, a first therapy or component (e.g. first DNA plasmid encoding an HBV antigen) can be administered prior to (e.g., 5 minutes to one hour before), concomitantly with or simultaneously with, or subsequent to (e.g., 5 minutes to one hour after) the administration of a second therapy or component (e.g., second DNA plasmid encoding an HBV antigen), and/or a third therapy or component (e.g., a pyridopyrimidine compound (i.e., a pyridopyrimidine derivative)). In some embodiments, a first therapy or component (e.g. first DNA plasmid encoding an HBV antigen), a second therapy or component (e.g., second DNA plasmid encoding an HBV antigen), and a third therapy or component (e.g., a pyridopyrimidine compound (i.e., a pyridopyrimidine derivative)) are administered in the same composition. In other embodiments, a first therapy or component (e.g. first DNA plasmid encoding an HBV antigen), a second therapy or component (e.g., second DNA plasmid encoding an HBV antigen), and a third therapy or component (e.g., a pyridopyrimidine compound (i.e., a pyridopyrimidine derivative)) are administered in separate compositions, such as two or three separate compositions.

As used herein, a “non-naturally occurring” nucleic acid or polypeptide, refers to a nucleic acid or polypeptide that does not occur in nature. A “non-naturally occurring” nucleic acid or polypeptide can be synthesized, treated, fabricated, and/or otherwise manipulated in a laboratory and/or manufacturing setting. In some cases, a non-naturally occurring nucleic acid or polypeptide can comprise a naturally-occurring nucleic acid or polypeptide that is treated, processed, or manipulated to exhibit properties that were not present in the naturally-occurring nucleic acid or polypeptide, prior to treatment. As used herein, a “non-naturally occurring” nucleic acid or polypeptide can be a nucleic acid or polypeptide isolated or separated from the natural source in which it was discovered, and it lacks covalent bonds to sequences with which it was associated in the natural source. A “non-naturally occurring” nucleic acid or polypeptide can be made recombinantly or via other methods, such as chemical synthesis.

As used herein, “subject” means any animal, preferably a mammal, most preferably a human, to whom will be or has been treated by a method according to an embodiment of the application. The term “mammal” as used herein, encompasses any mammal. Examples of mammals include, but are not limited to, cows, horses, sheep, pigs, cats, dogs, mice, rats, rabbits, guinea pigs, non-human primates (NHPs) such as monkeys or apes, humans, etc., more preferably a human.

As used herein, the term “operably linked” refers to a linkage or a juxtaposition wherein the components so described are in a relationship permitting them to function in their intended manner. For example, a regulatory sequence operably linked to a nucleic acid sequence of interest is capable of directing the transcription of the nucleic acid sequence of interest, or a signal sequence operably linked to an amino acid sequence of interest is capable of secreting or translocating the amino acid sequence of interest over a membrane.

In an attempt to help the reader of the application, the description has been separated in various paragraphs or sections, or is directed to various embodiments of the application. These separations should not be considered as disconnecting the substance of a paragraph or section or embodiments from the substance of another paragraph or section or embodiments. To the contrary, one skilled in the art will understand that the description has broad application and encompasses all the combinations of the various sections, paragraphs and sentences that can be contemplated. The discussion of any embodiment is meant only to be exemplary and is not intended to suggest that the scope of the disclosure, including the claims, is limited to these examples. For example, while embodiments of HBV vectors of the application (e.g., plasmid DNA or viral vectors) described herein may contain particular components, including, but not limited to, certain promoter sequences, enhancer or regulatory sequences, signal peptides, coding sequence of an HBV antigen, polyadenylation signal sequences, etc. arranged in a particular order, those having ordinary skill in the art will appreciate that the concepts disclosed herein may equally apply to other components arranged in other orders that can be used in HBV vectors of the application. The application contemplates use of any of the applicable components in any combination having any sequence that can be used in HBV vectors of the application, whether or not a particular combination is expressly described. The invention generally relates to a therapeutic combination comprising one or more HBV antigens and a pyridopyrimidine derivative.

### Hepatitis B Virus (HBV)

As used herein “hepatitis B virus” or “HBV” refers to a virus of the hepadnaviridae family. HBV is a small (e.g., 3.2 kb) hepatotropic DNA virus that encodes four open reading frames and seven proteins. The seven proteins encoded by HBV include small (S), medium (M), and large (L) surface antigen (HBsAg) or envelope (Env) proteins, pre-Core protein, core protein, viral polymerase (Pol), and HBx protein. HBV expresses three surface antigens, or envelope proteins, L, M, and S, with S being the smallest and L being the largest. The extra domains in the M and L proteins are named Pre-S2 and Pre-S1, respectively. Core protein is the subunit of the viral nucleocapsid. Pol is needed for synthesis of viral DNA (reverse transcriptase, RNaseH, and primer), which takes place in nucleocapsids localized to the cytoplasm of infected hepatocytes. PreCore is the core protein with an N-terminal signal peptide and is proteolytically processed at its N and C termini before secretion from infected cells, as the so-called hepatitis B e-antigen (HBeAg). HBx protein is required for efficient transcription of covalently closed circular DNA (cccDNA). HBx is not a viral structural protein. All viral proteins of HBV have their own mRNA except for core and polymerase, which share an mRNA. With the exception of the protein pre-Core, none of the HBV viral proteins are subject to post-translational proteolytic processing.

The HBV virion contains a viral envelope, nucleocapsid, and single copy of the partially double-stranded DNA genome. The nucleocapsid comprises 120 dimers of core protein and is covered by a capsid membrane embedded with the S, M, and L viral envelope or surface antigen proteins. After entry into the cell, the virus is uncoated and the capsid-containing relaxed circular DNA (rcDNA) with covalently bound viral polymerase migrates to the nucleus. During that process, phosphorylation of the core protein induces structural changes, exposing a nuclear localization signal enabling interaction of the capsid with so-called importins. These importins mediate binding of the core protein to nuclear pore complexes upon which the capsid disassembles and polymerase/rcDNA complex is released into the nucleus. Within the nucleus the rcDNA becomes deproteinized (removal of polymerase) and is converted by host DNA repair machinery to a covalently closed circular DNA (cccDNA) genome from which overlapping transcripts encode for HBeAg, HBsAg, Core protein, viral polymerase and HBx protein. Core protein, viral polymerase, and pre-genomic RNA (pgRNA) associate in the cytoplasm and self-assemble into immature pgRNA-containing capsid particles, which further convert into mature rcDNA-capsids and function as a common intermediate that is either enveloped and secreted as infectious virus particles or transported back to the nucleus to replenish and maintain a stable cccDNA pool.

To date, HBV is divided into four serotypes (adr, adw, ayr, ayw) based on antigenic epitopes present on the envelope proteins, and into eight genotypes (A, B, C, D, E, F, G, and H) based on the sequence of the viral genome. The HBV genotypes are distributed over different geographic regions. For example, the most prevalent genotypes in Asia are genotypes B and C. Genotype D is dominant in Africa, the Middle East, and India, whereas genotype A is widespread in Northern Europe, sub-Saharan Africa, and West Africa.

### HBV Antigens

As used herein, the terms “HBV antigen,” “antigenic polypeptide of HBV,” “HBV antigenic polypeptide,” “HBV antigenic protein,” “HBV immunogenic polypeptide,” and “HBV immunogen” all refer to a polypeptide capable of inducing an immune response, e.g., a humoral and/or cellular mediated response, against an HBV in a subject. The HBV antigen can be a polypeptide of HBV, a fragment or epitope thereof, or a combination of multiple HBV polypeptides, portions or derivatives thereof. An HBV antigen is capable of raising in a host a protective immune response, e.g., inducing an immune response against a viral disease or infection, and/or producing an immunity (i.e., vaccinates) in a subject against a viral disease or infection, that protects the subject against the viral disease or infection. For example, an HBV antigen can comprise a polypeptide or immunogenic fragment(s) thereof from any HBV protein, such as HBeAg, pre-core protein, HBsAg (S, M, or L proteins), core protein, viral polymerase, or HBx protein derived from any HBV genotype, e.g., genotype A, B, C, D, E, F, G, and/or H, or combination thereof.

### (1) HBV Core Antigen

As used herein, each of the terms “HBV core antigen,” “HBc” and “core antigen” refers to an HBV antigen capable of inducing an immune response, e.g., a humoral and/or cellular mediated response, against an HBV core protein in a subject. Each of the terms “core,” “core polypeptide,” and “core protein” refers to the HBV viral core protein. Full-length core antigen is typically 183 amino acids in length and includes an assembly domain (amino acids 1 to 149) and a nucleic acid binding domain (amino acids 150 to 183). The 34-residue nucleic acid binding domain is required for pre-genomic RNA encapsidation. This domain also functions as a nuclear import signal. It comprises 17 arginine residues and is highly basic, consistent with its function. HBV core protein is dimeric in solution, with the dimers self-assembling into icosahedral capsids. Each dimer of core protein has four α-helix bundles flanked by an α-helix domain on either side. Truncated HBV core proteins lacking the nucleic acid binding domain are also capable of forming capsids.

In an embodiment of the application, an HBV antigen is a truncated HBV core antigen. As used herein, a “truncated HBV core antigen,” refers to an HBV antigen that does not contain the entire length of an HBV core protein, but is capable of inducing an immune response against the HBV core protein in a subject. For example, an HBV core antigen can be modified to delete one or more amino acids of the highly positively charged (arginine rich) C-terminal nucleic acid binding domain of the core antigen, which typically contains seventeen arginine (R) residues. A truncated HBV core antigen of the application is preferably a C-terminally truncated HBV core protein which does not comprise the HBV core nuclear import signal and/or a truncated HBV core protein from which the C-terminal HBV core nuclear import signal has been deleted. In an embodiment, a truncated HBV core antigen comprises a deletion in the C-terminal nucleic acid binding domain, such as a deletion of 1 to 34 amino acid residues of the C-terminal nucleic acid binding domain, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 amino acid residues, preferably a deletion of all 34 amino acid residues. In a preferred embodiment, a truncated HBV core antigen comprises a deletion in the C-terminal nucleic acid binding domain, preferably a deletion of all 34 amino acid residues.

An HBV core antigen of the application can be a consensus sequence derived from multiple HBV genotypes (e.g., genotypes A, B, C, D, E, F, G, and H). As used herein, “consensus sequence” means an artificial sequence of amino acids based on an alignment of amino acid sequences of homologous proteins, e.g., as determined by an alignment (e.g., using Clustal Omega) of amino acid sequences of homologous proteins. It can be the calculated order of most frequent amino acid residues, found at each position in a sequence alignment, based upon sequences of HBV antigens (e.g., core, pol, etc.) from at least 100 natural HBV isolates. A consensus sequence can be non-naturally occurring and different from the native viral sequences. Consensus sequences can be designed by aligning multiple HBV antigen sequences from different sources using a multiple sequence alignment tool, and at variable alignment positions, selecting the most frequent amino acid. Preferably, a consensus sequence of an HBV antigen is derived from HBV genotypes B, C, and D. The term “consensus antigen” is used to refer to an antigen having a consensus sequence.

An exemplary truncated HBV core antigen according to the application lacks the nucleic acid binding function, and is capable of inducing an immune response in a mammal against at least two HBV genotypes. Preferably a truncated HBV core antigen is capable of inducing a T cell response in a mammal against at least HBV genotypes B, C and D. More preferably, a truncated HBV core antigen is capable of inducing a CD8 T cell response in a human subject against at least HBV genotypes A, B, C and D.

Preferably, an HBV core antigen of the application is a consensus antigen, preferably a consensus antigen derived from HBV genotypes B, C, and D, more preferably a truncated consensus antigen derived from HBV genotypes B, C, and D. An exemplary truncated HBV core consensus antigen according to the application consists of an amino acid sequence that is at least 90% identical to SEQ ID NO: 2 or SEQ ID NO: 4, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9%, or 100% identical to SEQ ID NO: 2 or SEQ ID NO: 4. SEQ ID NO: 2 and SEQ ID NO: 4 are core consensus antigens derived from HBV genotypes B, C, and D. SEQ ID NO: 2 and SEQ ID NO: 4 each contain a 34-amino acid C-terminal deletion of the highly positively charged (arginine rich) nucleic acid binding domain of the native core antigen.

In one embodiment of the application, an HBV core antigen is a truncated HBV antigen consisting of the amino acid sequence of SEQ ID NO: 2. In another embodiment, an HBV core antigen is a truncated HBV antigen consisting of the amino acid sequence of SEQ ID NO: 4. In another embodiment, an HBV core antigen further contains a signal sequence operably linked to the N-terminus of a mature HBV core antigen sequence, such as the amino acid sequence of SEQ ID NO: 2 or SEQ ID NO: 4. Preferably, the signal sequence has the amino acid sequence of SEQ ID NO: 9 or SEQ ID NO: 15.

### (2) HBV Polymerase Antigen

As used herein, the term “HBV polymerase antigen,” “HBV Pol antigen” or “HBV pol antigen” refers to an HBV antigen capable of inducing an immune response, e.g., a humoral and/or cellular mediated response, against an HBV polymerase in a subject. Each of the terms “polymerase,” “polymerase polypeptide,” “Pol” and “pol” refers to the HBV viral DNA polymerase. The HBV viral DNA polymerase has four domains, including, from the N terminus to the C terminus, a terminal protein (TP) domain, which acts as a primer for minus-strand DNA synthesis; a spacer that is nonessential for the polymerase functions; a reverse transcriptase (RT) domain for transcription; and an RNase H domain.

In an embodiment of the application, an HBV antigen comprises an HBV Pol antigen, or any immunogenic fragment or combination thereof. An HBV Pol antigen can contain further modifications to improve immunogenicity of the antigen, such as by introducing mutations into the active sites of the polymerase and/or RNase domains to decrease or substantially eliminate certain enzymatic activities.

Preferably, an HBV Pol antigen of the application does not have reverse transcriptase activity and RNase H activity, and is capable of inducing an immune response in a mammal against at least two HBV genotypes. Preferably, an HBV Pol antigen is capable of inducing a T cell response in a mammal against at least HBV genotypes B, C and D. More preferably, an HBV Pol antigen is capable of inducing a CD8 T cell response in a human subject against at least HBV genotypes A, B, C and D.

Thus, in some embodiments, an HBV Pol antigen is an inactivated Pol antigen. In an embodiment, an inactivated HBV Pol antigen comprises one or more amino acid mutations in the active site of the polymerase domain. In another embodiment, an inactivated HBV Pol antigen comprises one or more amino acid mutations in the active site of the RNaseH domain. In a preferred embodiment, an inactivated HBV pol antigen comprises one or more amino acid mutations in the active site of both the polymerase domain and the RNaseH domain. For example, the “YXDD” motif in the polymerase domain of an HBV pol antigen that can be required for nucleotide/metal ion binding can be mutated, e.g., by replacing one or more of the aspartate residues (D) with asparagine residues (N), eliminating or reducing metal coordination function, thereby decreasing or substantially eliminating reverse transcriptase function. Alternatively, or in addition to mutation of the “YXDD” motif, the “DEDD” motif in the RNaseH domain of an HBV pol antigen required for Mg2+ coordination can be mutated, e.g., by replacing one or more aspartate residues (D) with asparagine residues (N) and/or replacing the glutamate residue (E) with glutamine (Q), thereby decreasing or substantially eliminating RNaseH function. In a particular embodiment, an HBV pol antigen is modified by (1) mutating the aspartate residues (D) to asparagine residues (N) in the “YXDD” motif of the polymerase domain; and (2) mutating the first aspartate residue (D) to an asparagine residue (N) and the first glutamate residue (E) to a glutamine residue (N) in the “DEDD” motif of the RNaseH domain, thereby decreasing or substantially eliminating both the reverse transcriptase and RNaseH functions of the pol antigen.

In a preferred embodiment of the application, an HBV pol antigen is a consensus antigen, preferably a consensus antigen derived from HBV genotypes B, C, and D, more preferably an inactivated consensus antigen derived from HBV genotypes B, C, and D. An exemplary HBV pol consensus antigen according to the application comprises an amino acid sequence that is at least 90% identical to SEQ ID NO: 7, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 7, preferably at least 98% identical to SEQ ID NO: 7, such as at least 98%, 98.5%, 99%, 99.1%, 99.2%, 99.3%, 99.4%, 99.5%, 99.6%, 99.7%, 99.8%, 99.9% or 100% identical to SEQ ID NO: 7. SEQ ID NO: 7 is a pol consensus antigen derived from HBV genotypes B, C, and D comprising four mutations located in the active sites of the polymerase and RNaseH domains. In particular, the four mutations include mutation of the aspartic acid residues (D) to asparagine residues (N) in the “YXDD” motif of the polymerase domain; and mutation of the first aspartate residue (D) to an asparagine residue (N) and mutation of the glutamate residue (E) to a glutamine residue (Q) in the “DEDD” motif of the RNaseH domain.

In a particular embodiment of the application, an HBV pol antigen comprises the amino acid sequence of SEQ ID NO: 7. In other embodiments of the application, an HBV pol antigen consists of the amino acid sequence of SEQ ID NO: 7. In a further embodiment, an HBV pol antigen further contains a signal sequence operably linked to the N-terminus of a mature HBV pol antigen sequence, such as the amino acid sequence of SEQ ID NO: 7. Preferably, the signal sequence has the amino acid sequence of SEQ ID NO: 9 or SEQ ID NO: 15.

### (3) Fusion of HBV Core Antigen and HBV Polymerase Antigen

As used herein the term “fusion protein” or “fusion” refers to a single polypeptide chain having at least two polypeptide domains that are not normally present in a single, natural polypeptide.

In an embodiment of the application, an HBV antigen comprises a fusion protein comprising a truncated HBV core antigen operably linked to an HBV Pol antigen, or an HBV Pol antigen operably linked to a truncated HBV core antigen, preferably via a linker.

For example, in a fusion protein containing a first polypeptide and a second heterologous polypeptide, a linker serves primarily as a spacer between the first and second polypeptides. In an embodiment, a linker is made up of amino acids linked together by peptide bonds, preferably from 1 to 20 amino acids linked by peptide bonds, wherein the amino acids are selected from the 20 naturally occurring amino acids. In an embodiment, the 1 to 20 amino acids are selected from glycine, alanine, proline, asparagine, glutamine, and lysine. Preferably, a linker is made up of a majority of amino acids that are sterically unhindered, such as glycine and alanine. Exemplary linkers are polyglycines, particularly (Gly)5, (Gly)8; poly(Gly-Ala), and polyalanines. One exemplary suitable linker as shown in the Examples below is (AlaGly)n, wherein n is an integer of 2 to 5.

Preferably, a fusion protein of the application is capable of inducing an immune response in a mammal against HBV core and HBV Pol of at least two HBV genotypes. Preferably, a fusion protein is capable of inducing a T cell response in a mammal against at least HBV genotypes B, C and D. More preferably, the fusion protein is capable of inducing a CD8 T cell response in a human subject against at least HBV genotypes A, B, C and D.

In an embodiment of the application, a fusion protein comprises a truncated HBV core antigen having an amino acid sequence at least 90%, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9%, or 100% identical to SEQ ID NO: 2 or SEQ ID NO: 4, a linker, and an HBV Pol antigen having an amino acid sequence at least 90%, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9%, or 100%, identical to SEQ ID NO: 7.

In a preferred embodiment of the application, a fusion protein comprises a truncated HBV core antigen consisting of the amino acid sequence of SEQ ID NO: 2 or SEQ ID NO: 4, a linker comprising (AlaGly)n, wherein n is an integer of 2 to 5, and an HBV Pol antigen having the amino acid sequence of SEQ ID NO: 7. More preferably, a fusion protein according to an embodiment of the application comprises the amino acid sequence of SEQ ID NO: 16.

In one embodiment of the application, a fusion protein further comprises a signal sequence operably linked to the N-terminus of the fusion protein. Preferably, the signal sequence has the amino acid sequence of SEQ ID NO: 9 or SEQ ID NO: 15. In one embodiment, a fusion protein comprises the amino acid sequence of SEQ ID NO: 17.

Additional disclosure on HBV vaccines that can be used for the present invention are described in U.S. patent application Ser. No. 16/223,251, filed Dec. 18, 2018, the contents of the application, more preferably the examples of the application, are hereby incorporated by reference in their entireties.

### Polynucleotides and Vectors

In another general aspect, the application provides a non-naturally occurring nucleic acid molecule encoding an HBV antigen useful for an invention according to embodiments of the application, and vectors comprising the non-naturally occurring nucleic acid. A first or second non-naturally occurring nucleic acid molecule can comprise any polynucleotide sequence encoding an HBV antigen useful for the application, which can be made using methods known in the art in view of the present disclosure. Preferably, a first or second polynucleotide encodes at least one of a truncated HBV core antigen and an HBV polymerase antigen of the application. A polynucleotide can be in the form of RNA or in the form of DNA obtained by recombinant techniques (e.g., cloning) or produced synthetically (e.g., chemical synthesis). The DNA can be single-stranded or double-stranded, or can contain portions of both double-stranded and single-stranded sequence. The DNA can, for example, comprise genomic DNA, cDNA, or combinations thereof. The polynucleotide can also be a DNA/RNA hybrid. The polynucleotides and vectors of the application can be used for recombinant protein production, expression of the protein in host cell, or the production of viral particles. Preferably, a polynucleotide is DNA.

In an embodiment of the application, a first non-naturally occurring nucleic acid molecule comprises a first polynucleotide sequence encoding a truncated HBV core antigen consisting of an amino acid sequence that is at least 90% identical to SEQ ID NO: 2 or SEQ ID NO: 4, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 2, preferably 98%, 99% or 100% identical to SEQ ID NO: 2 or SEQ ID NO: 4. In a particular embodiment of the application, a first non-naturally occurring nucleic acid molecule comprises a first polynucleotide sequence encoding a truncated HBV core antigen consisting the amino acid sequence of SEQ ID NO: 2 or SEQ ID NO: 4.

Examples of polynucleotide sequences of the application encoding a truncated HBV core antigen consisting of the amino acid sequence of SEQ ID NO: 2 or SEQ ID NO: 4 include, but are not limited to, a polynucleotide sequence at least 90% identical to SEQ ID NO: 1 or SEQ ID NO: 3, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 1 or SEQ ID NO: 3, preferably 98%, 99% or 100% identical to SEQ ID NO: 1 or SEQ ID NO: 3. Exemplary non-naturally occurring nucleic acid molecules encoding a truncated HBV core antigen have the polynucleotide sequence of SEQ ID NOs: 1 or 3.

In another embodiment, a first non-naturally occurring nucleic acid molecule further comprises a coding sequence for a signal sequence that is operably linked to the N-terminus of the HBV core antigen sequence. Preferably, the signal sequence has the amino acid sequence of SEQ ID NO: 9 or SEQ ID NO: 15. More preferably, the coding sequence for a signal sequence comprises the polynucleotide sequence of SEQ ID NO: 8 or SEQ ID NO: 14.

In an embodiment of the application, a second non-naturally occurring nucleic acid molecule comprises a second polynucleotide sequence encoding an HBV polymerase antigen comprising an amino acid sequence that is at least 90% identical to SEQ ID NO: 7, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 7, preferably 100% identical to SEQ ID NO: 7. In a particular embodiment of the application, a second non-naturally occurring nucleic acid molecule comprises a second polynucleotide sequence encoding an HBV polymerase antigen consisting of the amino acid sequence of SEQ ID NO: 7.

Examples of polynucleotide sequences of the application encoding an HBV Pol antigen comprising the amino acid sequence of at least 90% identical to SEQ ID NO: 7 include, but are not limited to, a polynucleotide sequence at least 90% identical to SEQ ID NO: 5 or SEQ ID NO: 6, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 5 or SEQ ID NO: 6, preferably 98%, 99% or 100% identical to SEQ ID NO: 5 or SEQ ID NO: 6. Exemplary non-naturally occurring nucleic acid molecules encoding an HBV pol antigen have the polynucleotide sequence of SEQ ID NOs: 5 or 6.

In another embodiment, a second non-naturally occurring nucleic acid molecule further comprises a coding sequence for a signal sequence that is operably linked to the N-terminus of the HBV pol antigen sequence, such as the amino acid sequence of SEQ ID NO: 7. Preferably, the signal sequence has the amino acid sequence of SEQ ID NO: 9 or SEQ ID NO: 15. More preferably, the coding sequence for a signal sequence comprises the polynucleotide sequence of SEQ ID NO: 8 or SEQ ID NO: 14.

In another embodiment of the application, a non-naturally occurring nucleic acid molecule encodes an HBV antigen fusion protein comprising a truncated HBV core antigen operably linked to an HBV Pol antigen, or an HBV Pol antigen operably linked to a truncated HBV core antigen. In a particular embodiment, a non-naturally occurring nucleic acid molecule of the application encodes a truncated HBV core antigen consisting of an amino acid sequence that is at least 90% identical to SEQ ID NO: 2 or SEQ ID NO: 4, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 2 or SEQ ID NO: 4, preferably 100% identical to SEQ ID NO: 2 or SEQ ID NO: 4, more preferably 100% identical to SEQ ID NO: 2 or SEQ ID NO:4; a linker; and an HBV polymerase antigen comprising an amino acid sequence that is at least 90% identical to SEQ ID NO: 7, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 7, preferably 98%, 99% or 100% identical to SEQ ID NO: 7. In a particular embodiment of the application, a non-naturally occurring nucleic acid molecule encodes a fusion protein comprising a truncated HBV core antigen consisting of the amino acid sequence of SEQ ID NO: 2 or SEQ ID NO: 4, a linker comprising (AlaGly)n, wherein n is an integer of 2 to 5; and an HBV Pol antigen comprising the amino acid sequence of SEQ ID NO: 7. In a particular embodiment of the application, a non-naturally occurring nucleic acid molecule encodes an HBV antigen fusion protein comprising the amino acid sequence of SEQ ID NO: 16.

Examples of polynucleotide sequences of the application encoding an HBV antigen fusion protein include, but are not limited to, a polynucleotide sequence at least 90% identical to SEQ ID NO: 1 or SEQ ID NO: 3, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 1 or SEQ ID NO: 3, preferably 98%, 99% or 100% identical to SEQ ID NO: 1 or SEQ ID NO: 3, operably linked to a linker coding sequence at least 90% identical to SEQ ID NO: 11, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 11, preferably 98%, 99% or 100% identical to SEQ ID NO: 11, which is further operably linked a polynucleotide sequence at least 90% identical to SEQ ID NO: 5 or SEQ ID NO: 6, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 5 or SEQ ID NO: 6, preferably 98%, 99% or 100% identical to SEQ ID NO: 5 or SEQ ID NO: 6. In particular embodiments of the application, a non-naturally occurring nucleic acid molecule encoding an HBV antigen fusion protein comprises SEQ ID NO: 1 or SEQ ID NO: 3, operably linked to SEQ ID NO: 11, which is further operably linked to SEQ ID NO: 5 or SEQ ID NO: 6.

In another embodiment, a non-naturally occurring nucleic acid molecule encoding an HBV fusion further comprises a coding sequence for a signal sequence that is operably linked to the N-terminus of the HBV fusion sequence, such as the amino acid sequence of SEQ ID NO: 16. Preferably, the signal sequence has the amino acid sequence of SEQ ID NO: 9 or SEQ ID NO: 15. More preferably, the coding sequence for a signal sequence comprises the polynucleotide sequence of SEQ ID NO: 8 or SEQ ID NO: 14. In one embodiment, the encoded fusion protein with the signal sequence comprises the amino acid sequence of SEQ ID NO: 17.

The application also relates to a vector comprising the first and/or second non-naturally occurring nucleic acid molecules. As used herein, a “vector” is a nucleic acid molecule used to carry genetic material into another cell, where it can be replicated and/or expressed. Any vector known to those skilled in the art in view of the present disclosure can be used. Examples of vectors include, but are not limited to, plasmids, viral vectors (bacteriophage, animal viruses, and plant viruses), cosmids, and artificial chromosomes (e.g., YACs). Preferably, a vector is a DNA plasmid. A vector can be a DNA vector or an RNA vector. One of ordinary skill in the art can construct a vector of the application through standard recombinant techniques in view of the present disclosure.

A vector of the application can be an expression vector. As used herein, the term “expression vector” refers to any type of genetic construct comprising a nucleic acid coding for an RNA capable of being transcribed. Expression vectors include, but are not limited to, vectors for recombinant protein expression, such as a DNA plasmid or a viral vector, and vectors for delivery of nucleic acid into a subject for expression in a tissue of the subject, such as a DNA plasmid or a viral vector. It will be appreciated by those skilled in the art that the design of the expression vector can depend on such factors as the choice of the host cell to be transformed, the level of expression of protein desired, etc.

Vectors of the application can contain a variety of regulatory sequences. As used herein, the term “regulatory sequence” refers to any sequence that allows, contributes or modulates the functional regulation of the nucleic acid molecule, including replication, duplication, transcription, splicing, translation, stability and/or transport of the nucleic acid or one of its derivative (i.e. mRNA) into the host cell or organism. In the context of the disclosure, this term encompasses promoters, enhancers and other expression control elements (e.g., polyadenylation signals and elements that affect mRNA stability).

In some embodiments of the application, a vector is a non-viral vector. Examples of non-viral vectors include, but are not limited to, DNA plasmids, bacterial artificial chromosomes, yeast artificial chromosomes, bacteriophages, etc. Examples of non-viral vectors include, but are not limited to, RNA replicon, mRNA replicon, modified mRNA replicon or self-amplifying mRNA, closed linear deoxyribonucleic acid, e.g. a linear covalently closed DNA such as linear covalently closed double stranded DNA molecule. Preferably, a non-viral vector is a DNA plasmid. A “DNA plasmid”, which is used interchangeably with “DNA plasmid vector,” “plasmid DNA” or “plasmid DNA vector,” refers to a double-stranded and generally circular DNA sequence that is capable of autonomous replication in a suitable host cell. DNA plasmids used for expression of an encoded polynucleotide typically comprise an origin of replication, a multiple cloning site, and a selectable marker, which for example, can be an antibiotic resistance gene. Examples of DNA plasmids suitable that can be used include, but are not limited to, commercially available expression vectors for use in well-known expression systems (including both prokaryotic and eukaryotic systems), such as pSE420 (Invitrogen, San Diego, Calif.), which can be used for production and/or expression of protein in Escherichia coli; pYES2 (Invitrogen, Thermo Fisher Scientific), which can be used for production and/or expression in Saccharomyces cerevisiae strains of yeast; MAXBAC® complete baculovirus expression system (Thermo Fisher Scientific), which can be used for production and/or expression in insect cells; pcDNA™ or pcDNA3™ (Life Technologies, Thermo Fisher Scientific), which can be used for high level constitutive protein expression in mammalian cells; and pVAX or pVAX-1 (Life Technologies, Thermo Fisher Scientific), which can be used for high-level transient expression of a protein of interest in most mammalian cells. The backbone of any commercially available DNA plasmid can be modified to optimize protein expression in the host cell, such as to reverse the orientation of certain elements (e.g., origin of replication and/or antibiotic resistance cassette), replace a promoter endogenous to the plasmid (e.g., the promoter in the antibiotic resistance cassette), and/or replace the polynucleotide sequence encoding transcribed proteins (e.g., the coding sequence of the antibiotic resistance gene), by using routine techniques and readily available starting materials. (See e.g., Sambrook et al., Molecular Cloning a Laboratory Manual, Second Ed. Cold Spring Harbor Press (1989)).

Preferably, a DNA plasmid is an expression vector suitable for protein expression in mammalian host cells. Expression vectors suitable for protein expression in mammalian host cells include, but are not limited to, pcDNA™, pcDNA3™, pVAX, pVAX-1, ADVAX, NTC8454, etc. Preferably, an expression vector is based on pVAX-1, which can be further modified to optimize protein expression in mammalian cells. pVAX-1 is commonly used plasmid in DNA vaccines, and contains a strong human intermediate early cytomegalovirus (CMV-IE) promoter followed by the bovine growth hormone (bGH)-derived polyadenylation sequence (pA). pVAX-1 further contains a pUC origin of replication and kanamycin resistance gene driven by a small prokaryotic promoter that allows for bacterial plasmid propagation.

A vector of the application can also be a viral vector. In general, viral vectors are genetically engineered viruses carrying modified viral DNA or RNA that has been rendered non-infectious, but still contains viral promoters and transgenes, thus allowing for translation of the transgene through a viral promoter. Because viral vectors are frequently lacking infectious sequences, they require helper viruses or packaging lines for large-scale transfection. Examples of viral vectors that can be used include, but are not limited to, adenoviral vectors, adeno-associated virus vectors, pox virus vectors, enteric virus vectors, Venezuelan Equine Encephalitis virus vectors, Semliki Forest Virus vectors, Tobacco Mosaic Virus vectors, lentiviral vectors, etc. Examples of viral vectors that can be used include, but are not limited to, arenavirus viral vectors, replication-deficient arenavirus viral vectors or replication-competent arenavirus viral vectors, bi-segmented or tri-segmented arenavirus, infectious arenavirus viral vectors, nucleic acids which comprise an arenavirus genomic segment wherein one open reading frame of the genomic segment is deleted or functionally inactivated (and replaced by a nucleic acid encoding an HBV antigen as described herein), arenavirus such as lymphocytic choriomeningitidis virus (LCMV), e.g., clone 13 strain or MP strain, and arenavirus such as Junin virus e.g., Candid #1 strain. The vector can also be a non-viral vector.

Preferably, a viral vector is an adenovirus vector, e.g., a recombinant adenovirus vector. A recombinant adenovirus vector can for instance be derived from a human adenovirus (HAdV, or AdHu), or a simian adenovirus such as chimpanzee or gorilla adenovirus (ChAd, AdCh, or SAdV) or rhesus adenovirus (rhAd). Preferably, an adenovirus vector is a recombinant human adenovirus vector, for instance a recombinant human adenovirus serotype 26, or any one of recombinant human adenovirus serotype 5, 4, 35, 7, 48, etc. In other embodiments, an adenovirus vector is a rhAd vector, e.g. rhAd51, rhAd52 or rhAd53. A recombinant viral vector useful for the application can be prepared using methods known in the art in view of the present disclosure. For example, in view of the degeneracy of the genetic code, several nucleic acid sequences can be designed that encode the same polypeptide. A polynucleotide encoding an HBV antigen of the application can optionally be codon-optimized to ensure proper expression in the host cell (e.g., bacterial or mammalian cells). Codon-optimization is a technology widely applied in the art, and methods for obtaining codon-optimized polynucleotides will be well known to those skilled in the art in view of the present disclosure.

A vector of the application, e.g., a DNA plasmid or a viral vector (particularly an adenoviral vector), can comprise any regulatory elements to establish conventional function(s) of the vector, including but not limited to replication and expression of the HBV antigen(s) encoded by the polynucleotide sequence of the vector. Regulatory elements include, but are not limited to, a promoter, an enhancer, a polyadenylation signal, translation stop codon, a ribosome binding element, a transcription terminator, selection markers, origin of replication, etc. A vector can comprise one or more expression cassettes. An “expression cassette” is part of a vector that directs the cellular machinery to make RNA and protein. An expression cassette typically comprises three components: a promoter sequence, an open reading frame, and a 3′-untranslated region (UTR) optionally comprising a polyadenylation signal. An open reading frame (ORF) is a reading frame that contains a coding sequence of a protein of interest (e.g., HBV antigen) from a start codon to a stop codon. Regulatory elements of the expression cassette can be operably linked to a polynucleotide sequence encoding an HBV antigen of interest. As used herein, the term “operably linked” is to be taken in its broadest reasonable context, and refers to a linkage of polynucleotide elements in a functional relationship. A polynucleotide is “operably linked” when it is placed into a functional relationship with another polynucleotide. For instance, a promoter is operably linked to a coding sequence if it affects the transcription of the coding sequence. Any components suitable for use in an expression cassette described herein can be used in any combination and in any order to prepare vectors of the application.

A vector can comprise a promoter sequence, preferably within an expression cassette, to control expression of an HBV antigen of interest. The term “promoter” is used in its conventional sense, and refers to a nucleotide sequence that initiates the transcription of an operably linked nucleotide sequence. A promoter is located on the same strand near the nucleotide sequence it transcribes. Promoters can be a constitutive, inducible, or repressible. Promoters can be naturally occurring or synthetic. A promoter can be derived from sources including viral, bacterial, fungal, plants, insects, and animals. A promoter can be a homologous promoter (i.e., derived from the same genetic source as the vector) or a heterologous promoter (i.e., derived from a different vector or genetic source). For example, if the vector to be employed is a DNA plasmid, the promoter can be endogenous to the plasmid (homologous) or derived from other sources (heterologous). Preferably, the promoter is located upstream of the polynucleotide encoding an HBV antigen within an expression cassette.

Examples of promoters that can be used include, but are not limited to, a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter (CMV-IE), Epstein Barr virus (EBV) promoter, or a Rous sarcoma virus (RSV) promoter. A promoter can also be a promoter from a human gene such as human actin, human myosin, human hemoglobin, human muscle creatine, or human metalothionein. A promoter can also be a tissue specific promoter, such as a muscle or skin specific promoter, natural or synthetic.

Preferably, a promoter is a strong eukaryotic promoter, preferably a cytomegalovirus immediate early (CMV-IE) promoter. A nucleotide sequence of an exemplary CMV-IE promoter is shown in SEQ ID NO: 18 or SEQ ID NO: 19.

A vector can comprise additional polynucleotide sequences that stabilize the expressed transcript, enhance nuclear export of the RNA transcript, and/or improve transcriptional-translational coupling. Examples of such sequences include polyadenylation signals and enhancer sequences. A polyadenylation signal is typically located downstream of the coding sequence for a protein of interest (e.g., an HBV antigen) within an expression cassette of the vector. Enhancer sequences are regulatory DNA sequences that, when bound by transcription factors, enhance the transcription of an associated gene. An enhancer sequence is preferably located upstream of the polynucleotide sequence encoding an HBV antigen, but downstream of a promoter sequence within an expression cassette of the vector.

Any polyadenylation signal known to those skilled in the art in view of the present disclosure can be used. For example, the polyadenylation signal can be a SV40 polyadenylation signal, LTR polyadenylation signal, bovine growth hormone (bGH) polyadenylation signal, human growth hormone (hGH) polyadenylation signal, or human β-globin polyadenylation signal. Preferably, a polyadenylation signal is a bovine growth hormone (bGH) polyadenylation signal or a SV40 polyadenylation signal. A nucleotide sequence of an exemplary bGH polyadenylation signal is shown in SEQ ID NO: 20. A nucleotide sequence of an exemplary SV40 polyadenylation signal is shown in SEQ ID NO: 13.

Any enhancer sequence known to those skilled in the art in view of the present disclosure can be used. For example, an enhancer sequence can be human actin, human myosin, human hemoglobin, human muscle creatine, or a viral enhancer, such as one from CMV, HA, RSV, or EBV. Examples of particular enhancers include, but are not limited to, Woodchuck HBV Post-transcriptional regulatory element (WPRE), intron/exon sequence derived from human apolipoprotein A1 precursor (ApoAI), untranslated R-U5 domain of the human T-cell leukemia virus type 1 (HTLV-1) long terminal repeat (LTR), a splicing enhancer, a synthetic rabbit β-globin intron, or any combination thereof. Preferably, an enhancer sequence is a composite sequence of three consecutive elements of the untranslated R-U5 domain of HTLV-1 LTR, rabbit β-globin intron, and a splicing enhancer, which is referred to herein as “a triple enhancer sequence.” A nucleotide sequence of an exemplary triple enhancer sequence is shown in SEQ ID NO: 10. Another exemplary enhancer sequence is an ApoAI gene fragment shown in SEQ ID NO: 12.

A vector can comprise a polynucleotide sequence encoding a signal peptide sequence. Preferably, the polynucleotide sequence encoding the signal peptide sequence is located upstream of the polynucleotide sequence encoding an HBV antigen. Signal peptides typically direct localization of a protein, facilitate secretion of the protein from the cell in which it is produced, and/or improve antigen expression and cross-presentation to antigen-presenting cells. A signal peptide can be present at the N-terminus of an HBV antigen when expressed from the vector, but is cleaved off by signal peptidase, e.g., upon secretion from the cell. An expressed protein in which a signal peptide has been cleaved is often referred to as the “mature protein.” Any signal peptide known in the art in view of the present disclosure can be used. For example, a signal peptide can be a cystatin S signal peptide; an immunoglobulin (Ig) secretion signal, such as the Ig heavy chain gamma signal peptide SPIgG or the Ig heavy chain epsilon signal peptide SPIgE.

Preferably, a signal peptide sequence is a cystatin S signal peptide. Exemplary nucleic acid and amino acid sequences of a cystatin S signal peptide are shown in SEQ ID NOs: 8 and 9, respectively. Exemplary nucleic acid and amino acid sequences of an immunoglobulin secretion signal are shown in SEQ ID NOs: 14 and 15, respectively.

A vector, such as a DNA plasmid, can also include a bacterial origin of replication and an antibiotic resistance expression cassette for selection and maintenance of the plasmid in bacterial cells, e.g., E. coli. Bacterial origins of replication and antibiotic resistance cassettes can be located in a vector in the same orientation as the expression cassette encoding an HBV antigen, or in the opposite (reverse) orientation. An origin of replication (ORI) is a sequence at which replication is initiated, enabling a plasmid to reproduce and survive within cells. Examples of ORIs suitable for use in the application include, but are not limited to ColE1, pMB1, pUC, pSC101, R6K, and 15A, preferably pUC. An exemplary nucleotide sequence of a pUC ORI is shown in SEQ ID NO: 21.

Expression cassettes for selection and maintenance in bacterial cells typically include a promoter sequence operably linked to an antibiotic resistance gene. Preferably, the promoter sequence operably linked to an antibiotic resistance gene differs from the promoter sequence operably linked to a polynucleotide sequence encoding a protein of interest, e.g., HBV antigen. The antibiotic resistance gene can be codon optimized, and the sequence composition of the antibiotic resistance gene is normally adjusted to bacterial, e.g., E. coli, codon usage. Any antibiotic resistance gene known to those skilled in the art in view of the present disclosure can be used, including, but not limited to, kanamycin resistance gene (Kanr), ampicillin resistance gene (Ampr), and tetracycline resistance gene (Tetr), as well as genes conferring resistance to chloramphenicol, bleomycin, spectinomycin, carbenicillin, etc.

Preferably, an antibiotic resistance gene in the antibiotic expression cassette of a vector is a kanamycin resistance gene (Kanr). The sequence of Kanr gene is shown in SEQ ID NO: 22. Preferably, the Kanr gene is codon optimized. An exemplary nucleic acid sequence of a codon optimized Kanr gene is shown in SEQ ID NO: 23. The Kanr can be operably linked to its native promoter, or the Kanr gene can be linked to a heterologous promoter. In a particular embodiment, the Kanr gene is operably linked to the ampicillin resistance gene (Ampr) promoter, known as the bla promoter. An exemplary nucleotide sequence of a bla promoter is shown in SEQ ID NO: 24.

In a particular embodiment of the application, a vector is a DNA plasmid comprising an expression cassette including a polynucleotide encoding at least one of an HBV antigen selected from the group consisting of an HBV pol antigen comprising an amino acid sequence at least 90%, such as 90%, 91%, 92%, 93%, 94%, 95%, 96, 97%, preferably at least 98%, such as at least 98%, 98.5%, 99%, 99.1%, 99.2%, 99.3%, 99.4%, 99.5%, 99.6%, 99.7%, 99.8%, 99.9% or 100%, identical to SEQ ID NO: 7, and a truncated HBV core antigen consisting of the amino acid sequence at least 95%, such as 95%, 96, 97%, preferably at least 98%, such as at least 98%, 98.5%, 99%, 99.1%, 99.2%, 99.3%, 99.4%, 99.5%, 99.6%, 99.7%, 99.8%, 99.9% or 100%, identical of SEQ ID NO: 2 or SEQ ID NO: 4; an upstream sequence operably linked to the polynucleotide encoding the HBV antigen comprising, from 5′ end to 3′ end, a promoter sequence, preferably a CMV promoter sequence of SEQ ID NO: 18, an enhancer sequence, preferably a triple enhancer sequence of SEQ ID NO: 10, and a polynucleotide sequence encoding a signal peptide sequence, preferably a cystatin S signal peptide having the amino acid sequence of SEQ ID NO: 9; and a downstream sequence operably linked to the polynucleotide encoding the HBV antigen comprising a polyadenylation signal, preferably a bGH polyadenylation signal of SEQ ID NO: 20. Such vector further comprises an antibiotic resistance expression cassette including a polynucleotide encoding an antibiotic resistance gene, preferably a Kan^{r} gene, more preferably a codon optimized Kan^{r} gene of at least 90% identical to SEQ ID NO: 23, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 23, preferably 100% identical to SEQ ID NO: 23, operably linked to an Ampr (bla) promoter of SEQ ID NO: 24, upstream of and operably linked to the polynucleotide encoding the antibiotic resistance gene; and an origin of replication, preferably a pUC ori of SEQ ID NO: 21. Preferably, the antibiotic resistance cassette and the origin of replication are present in the plasmid in the reverse orientation relative to the HBV antigen expression cassette.

In another particular embodiment of the application, a vector is a viral vector, preferably an adenoviral vector, more preferably an Ad26 or Ad35 vector, comprising an expression cassette including a polynucleotide encoding at least one of an HBV antigen selected from the group consisting of an HBV pol antigen comprising an amino acid sequence at least 90%, such as 90%, 91%, 92%, 93%, 94%, 95%, 96, 97%, preferably at least 98%, such as at least 98%, 98.5%, 99%, 99.1%, 99.2%, 99.3%, 99.4%, 99.5%, 99.6%, 99.7%, 99.8%, 99.9% or 100%, identical to SEQ ID NO: 7, and a truncated HBV core antigen consisting of the amino acid sequence at least 95%, such as 95%, 96, 97%, preferably at least 98%, such as at least 98%, 98.5%, 99%, 99.1%, 99.2%, 99.3%, 99.4%, 99.5%, 99.6%, 99.7%, 99.8%, 99.9% or 100%, identical of SEQ ID NO: 2 or SEQ ID NO: 4; an upstream sequence operably linked to the polynucleotide encoding the HBV antigen comprising, from 5′ end to 3′ end, a promoter sequence, preferably a CMV promoter sequence of SEQ ID NO: 19, an enhancer sequence, preferably an ApoAI gene fragment sequence of SEQ ID NO: 12, and a polynucleotide sequence encoding a signal peptide sequence, preferably an immunoglobulin secretion signal having the amino acid sequence of SEQ ID NO: 15; and a downstream sequence operably linked to the polynucleotide encoding the HBV antigen comprising a polyadenylation signal, preferably a SV40 polyadenylation signal of SEQ ID NO: 13.

In an embodiment of the application, a vector, such as a plasmid DNA vector or a viral vector (preferably an adenoviral vector, more preferably an Ad26 or Ad35 vector), encodes an HBV Pol antigen having the amino acid sequence of SEQ ID NO: 7. Preferably, the vector comprises a coding sequence for the HBV Pol antigen that is at least 90% identical to the polynucleotide sequence of SEQ ID NO: 5 or 6, such as 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 5 or 6, preferably 100% identical to SEQ ID NO: 5 or 6.

In an embodiment of the application, a vector, such as a plasmid DNA vector or a viral vector (preferably an adenoviral vector, more preferably an Ad26 or Ad35 vector), encodes a truncated HBV core antigen consisting of the amino acid sequence of SEQ ID NO: 2 or SEQ ID NO: 4. Preferably, the vector comprises a coding sequence for the truncated HBV core antigen that is at least 90% identical to the polynucleotide sequence of SEQ ID NO: 1 or SEQ ID NO: 3, such as 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 1 or SEQ ID NO: 3, preferably 100% identical to SEQ ID NO: 1 or SEQ ID NO: 3.

In yet another embodiment of the application, a vector, such as a plasmid DNA vector or a viral vector (preferably an adenoviral vector, more preferably an Ad26 or Ad35 vector), encodes a fusion protein comprising an HBV Pol antigen having the amino acid sequence of SEQ ID NO: 7 and a truncated HBV core antigen consisting of the amino acid sequence of SEQ ID NO: 1 or SEQ ID NO: 3. Preferably, the vector comprises a coding sequence for the fusion, which contains a coding sequence for the truncated HBV core antigen at least 90% identical to SEQ ID NO: 1 or SEQ ID NO: 3, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 1 or SEQ ID NO: 3, preferably 98%, 99% or 100% identical to SEQ ID NO: 1 or SEQ ID NO: 3, more preferably SEQ ID NO: 1 or SEQ ID NO: 3, operably linked to a coding sequence for the HBV Pol antigen at least 90% identical to SEQ ID NO: 5 or SEQ ID NO: 6, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 5 or SEQ ID NO: 6, preferably 98%, 99% or 100% identical to SEQ ID NO: 5 or SEQ ID NO: 6, more preferably SEQ ID NO: 5 or SEQ ID NO: 6. Preferably, the coding sequence for the truncated HBV core antigen is operably linked to the coding sequence for the HBV Pol antigen via a coding sequence for a linker at least 90% identical to SEQ ID NO: 11, such as at least 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 11, preferably 98%, 99% or 100% identical to SEQ ID NO: 11. In particular embodiments of the application, a vector comprises a coding sequence for the fusion having SEQ ID NO: 1 or SEQ ID NO: 3 operably linked to SEQ ID NO: 11, which is further operably linked to SEQ ID NO: 5 or SEQ ID NO: 6.

The polynucleotides and expression vectors encoding the HBV antigens of the application can be made by any method known in the art in view of the present disclosure. For example, a polynucleotide encoding an HBV antigen can be introduced or “cloned” into an expression vector using standard molecular biology techniques, e.g., polymerase chain reaction (PCR), etc., which are well known to those skilled in the art.

### Cells, Polypeptides and Antibodies

The application also provides cells, preferably isolated cells, comprising any of the polynucleotides and vectors described herein. The cells can, for instance, be used for recombinant protein production, or for the production of viral particles.

Embodiments of the application thus also relate to a method of making an HBV antigen of the application. The method comprises transfecting a host cell with an expression vector comprising a polynucleotide encoding an HBV antigen of the application operably linked to a promoter, growing the transfected cell under conditions suitable for expression of the HBV antigen, and optionally purifying or isolating the HBV antigen expressed in the cell. The HBV antigen can be isolated or collected from the cell by any method known in the art including affinity chromatography, size exclusion chromatography, etc. Techniques used for recombinant protein expression will be well known to one of ordinary skill in the art in view of the present disclosure. The expressed HBV antigens can also be studied without purifying or isolating the expressed protein, e.g., by analyzing the supernatant of cells transfected with an expression vector encoding the HBV antigen and grown under conditions suitable for expression of the HBV antigen.

Thus, also provided are non-naturally occurring or recombinant polypeptides comprising an amino acid sequence that is at least 90% identical to the amino acid sequence of SEQ ID NO: 2, SEQ ID NO: 4, or SEQ ID NO: 7. As described above and below, isolated nucleic acid molecules encoding these sequences, vectors comprising these sequences operably linked to a promoter, and compositions comprising the polypeptide, polynucleotide, or vector are also contemplated by the application.

In an embodiment of the application, a recombinant polypeptide comprises an amino acid sequence that is at least 90% identical to the amino acid sequence of SEQ ID NO: 2, such as 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 2. Preferably, a non-naturally occurring or recombinant polypeptide consists of SEQ ID NO: 2.

In another embodiment of the application, a non-naturally occurring or recombinant polypeptide comprises an amino acid sequence that is at least 90% identical to the amino acid sequence of SEQ ID NO: 4, such as 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 4. Preferably, a non-naturally occurring or recombinant polypeptide comprises SEQ ID NO: 4.

In another embodiment of the application, a non-naturally occurring or recombinant polypeptide comprises an amino acid sequence that is at least 90% identical to the amino acid sequence of SEQ ID NO: 7, such as 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% identical to SEQ ID NO: 7. Preferably, a non-naturally occurring or recombinant polypeptide consists of SEQ ID NO: 7.

Also provided are antibodies or antigen binding fragments thereof that specifically bind to a non-naturally occurring polypeptide of the application. In an embodiment of the application, an antibody specific to a non-naturally HBV antigen of the application does not bind specifically to another HBV antigen. For example, an antibody of the application that binds specifically to an HBV Pol antigen having the amino acid sequence of SEQ ID NO: 7 will not bind specifically to an HBV Pol antigen not having the amino acid sequence of SEQ ID NO: 7.

As used herein, the term “antibody” includes polyclonal, monoclonal, chimeric, humanized, Fv, Fab and F(ab′)2; bifunctional hybrid (e.g., Lanzavecchia et al., Eur. J. Immunol. 17:105, 1987), single-chain (Huston et al., Proc. Natl. Acad. Sci. USA 85:5879, 1988; Bird et al., Science 242:423, 1988); and antibodies with altered constant regions (e.g., U.S. Pat. No. 5,624,821).

As used herein, an antibody that “specifically binds to” an antigen refers to an antibody that binds to the antigen with a KD of 1×10⁻⁷ M or less. Preferably, an antibody that “specifically binds to” an antigen binds to the antigen with a KD of 1×10⁻⁸ M or less, more preferably 5×10⁻⁹ M or less, 1×10⁻⁹ M or less, 5×10⁻¹⁰ M or less, or 1×10⁻¹⁰ M or less. The term “KD” refers to the dissociation constant, which is obtained from the ratio of Kd to Ka (i.e., Kd/Ka) and is expressed as a molar concentration (M). KD values for antibodies can be determined using methods in the art in view of the present disclosure. For example, the KD of an antibody can be determined by using surface plasmon resonance, such as by using a biosensor system, e.g., a Biacore® system, or by using bio-layer interferometry technology, such as a Octet RED96 system.

The smaller the value of the KD of an antibody, the higher affinity that the antibody binds to a target antigen.

### Compositions, Therapeutic Combinations, and Vaccines

The application also relates to compositions, therapeutic combinations, more particularly kits, and vaccines comprising one or more HBV antigens, polynucleotides, and/or vectors encoding one or more HBV antigens according to the application. Any of the HBV antigens, polynucleotides (including RNA and DNA), and/or vectors of the application described herein can be used in the compositions, therapeutic combinations or kits, and vaccines of the application.

In an embodiment of the application, a composition comprises an isolated or non-naturally occurring nucleic acid molecule (DNA or RNA) comprising polynucleotide sequence encoding a truncated HBV core antigen consisting of an amino acid sequence that is at least 90% identical to SEQ ID NO: 2 or SEQ ID NO: 4, or an HBV polymerase antigen comprising an amino acid sequence that is at least 90% identical to SEQ ID NO: 7, a vector comprising the isolated or non-naturally occurring nucleic acid molecule, and/or an isolated or non-naturally occurring polypeptide encoded by the isolated or non-naturally occurring nucleic acid molecule.

In an embodiment of the application, a composition comprises an isolated or non-naturally occurring nucleic acid molecule (DNA or RNA) comprising a polynucleotide sequence encoding an HBV Pol antigen comprising an amino acid sequence that is at least 90% identical to SEQ ID NO: 7, preferably 100% identical to SEQ ID NO: 7.

In an embodiment of the application, a composition comprises an isolated or non-naturally occurring nucleic acid molecule (DNA or RNA) encoding a truncated HBV core antigen consisting of an amino acid sequence that is at least 90% identical to SEQ ID NO: 2 or SEQ ID NO: 4, preferably 100% identical to SEQ ID NO: 2 or SEQ ID NO: 4.

In an embodiment of the application, a composition comprises an isolated or non-naturally occurring nucleic acid molecule (DNA or RNA) comprising a polynucleotide sequence encoding a truncated HBV core antigen consisting of an amino acid sequence that is at least 90% identical to SEQ ID NO: 2 or SEQ ID NO: 4, preferably 100% identical to SEQ ID NO: 2 or SEQ ID NO: 4; and an isolated or non-naturally occurring nucleic acid molecule (DNA or RNA) comprising a polynucleotide sequence encoding an HBV Pol antigen comprising an amino acid sequence that is at least 90% identical to SEQ ID NO: 7, preferably 100% identical to SEQ ID NO: 7. The coding sequences for the truncated HBV core antigen and the HBV Pol antigen can be present in the same isolated or non-naturally occurring nucleic acid molecule (DNA or RNA), or in two different isolated or non-naturally occurring nucleic acid molecules (DNA or RNA).

In an embodiment of the application, a composition comprises a vector, preferably a DNA plasmid or a viral vector (such as an adenoviral vector) comprising a polynucleotide encoding a truncated HBV core antigen consisting of an amino acid sequence that is at least 90% identical to SEQ ID NO: 2 or SEQ ID NO: 4, preferably 100% identical to SEQ ID NO: 2 or SEQ ID NO: 4.

In an embodiment of the application, a composition comprises a vector, preferably a DNA plasmid or a viral vector (such as an adenoviral vector), comprising a polynucleotide encoding an HBV Pol antigen comprising an amino acid sequence that is at least 90% identical to SEQ ID NO: 7, preferably 100% identical to SEQ ID NO: 7.

In an embodiment of the application, a composition comprises a vector, preferably a DNA plasmid or a viral vector (such as an adenoviral vector), comprising a polynucleotide encoding a truncated HBV core antigen consisting of an amino acid sequence that is at least 90% identical to SEQ ID NO: 2 or SEQ ID NO: 4, preferably 100% identical to SEQ ID NO: 2 or SEQ ID NO: 4; and a vector, preferably a DNA plasmid or a viral vector (such as an adenoviral vector), comprising a polynucleotide encoding an HBV Pol antigen comprising an amino acid sequence that is at least 90% identical to SEQ ID NO: 7, preferably 100% identical to SEQ ID NO: 7. The vector comprising the coding sequence for the truncated HBV core antigen and the vector comprising the coding sequence for the HBV Pol antigen can be the same vector, or two different vectors.

In an embodiment of the application, a composition comprises a vector, preferably a DNA plasmid or a viral vector (such as an adenoviral vector), comprising a polynucleotide encoding a fusion protein comprising a truncated HBV core antigen consisting of an amino acid sequence that is at least 90% identical to SEQ ID NO: 2 or SEQ ID NO: 4, preferably 100% identical to SEQ ID NO: 2 or SEQ ID NO: 4, operably linked to an HBV Pol antigen comprising an amino acid sequence that is at least 90% identical to SEQ ID NO: 7, preferably 100% identical to SEQ ID NO: 7, or vice versa. Preferably, the fusion protein further comprises a linker that operably links the truncated HBV core antigen to the HBV Pol antigen, or vice versa. Preferably, the linker has the amino acid sequence of (AlaGly)n, wherein n is an integer of 2 to 5.

In an embodiment of the application, a composition comprises an isolated or non-naturally occurring truncated HBV core antigen consisting of an amino acid sequence that is at least 90% identical to SEQ ID NO: 2 or SEQ ID NO: 4, preferably 100% identical to SEQ ID NO: 2 or SEQ ID NO: 4.

In an embodiment of the application, a composition comprises an isolated or non-naturally occurring HBV Pol antigen comprising an amino acid sequence that is at least 90% identical to SEQ ID NO: 7, preferably 100% identical to SEQ ID NO: 7.

In an embodiment of the application, a composition comprises an isolated or non-naturally occurring truncated HBV core antigen consisting of an amino acid sequence that is at least 90% identical to SEQ ID NO: 2 or SEQ ID NO: 4, preferably 100% identical to SEQ ID NO: 2 or SEQ ID NO: 4; and an isolated or non-naturally occurring HBV Pol antigen comprising an amino acid sequence that is at least 90% identical to SEQ ID NO: 7, preferably 100% identical to SEQ ID NO: 7.

In an embodiment of the application, a composition comprises an isolated or non-naturally occurring fusion protein comprising a truncated HBV core antigen consisting of an amino acid sequence that is at least 90% identical to SEQ ID NO: 2 or SEQ ID NO: 14, preferably 100% identical to SEQ ID NO: 2 or SEQ ID NO: 4, operably linked to an HBV Pol antigen comprising an amino acid sequence that is at least 90% identical to SEQ ID NO: 7, preferably 100% identical to SEQ ID NO: 7, or vice versa. Preferably, the fusion protein further comprises a linker that operably links the truncated HBV core antigen to the HBV Pol antigen, or vice versa. Preferably, the linker has the amino acid sequence of (AlaGly)n, wherein n is an integer of 2 to 5.

The application also relates to a therapeutic combination or a kit comprising polynucleotides expressing a truncated HBV core antigen and an HBV pol antigen according to embodiments of the application. Any polynucleotides and/or vectors encoding HBV core and pol antigens of the application described herein can be used in the therapeutic combinations or kits of the application.

According to embodiments of the application, a therapeutic combination or kit for use in treating an HBV infection in a subject in need thereof, comprises:

i) at least one of:
a) a truncated HBV core antigen consisting of an amino acid sequence that is at least 95% identical to SEQ ID NO: 2,
b) a first non-naturally occurring nucleic acid molecule comprising a first polynucleotide sequence encoding the truncated HBV core antigen,
c) an HBV polymerase antigen having an amino acid sequence that is at least 90% identical to SEQ ID NO: 7, wherein the HBV polymerase antigen does not have reverse transcriptase activity and RNase H activity, and
d) a second non-naturally occurring nucleic acid molecule comprising a second polynucleotide sequence encoding the HBV polymerase antigen; and
   ii) a benzazepine carboxamide compound of formula (K)

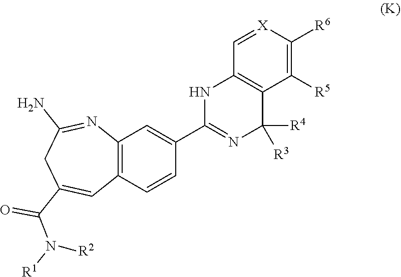

wherein
R¹ is C₃₋₇-alkyl,
R² is C₃₋₇-alkyl or C₃₋₇-cycloalkyl-C₁₋₇-alkyl,
R³ is hydrogen or C₁₋₇-alkyl,
R⁴ is hydrogen or C₁₋₇-alkyl,
R⁵ is selected from the group consisting of hydrogen, halogen, C₁₋₇-alkyl and C₁₋₇-alkoxy,
R⁶ is selected from the group consisting of hydrogen, halogen, C₁₋₇-alkyl and C₁₋₇-alkoxy, and

### X is N or CR⁷, and

wherein R⁷ is selected from the group consisting of hydrogen, halogen, C₁₋₇-alkyl and C₁₋₇-alkoxy)
or pharmaceutically acceptable salts thereof;
or a pyridopyrimidine compound of formula (J)

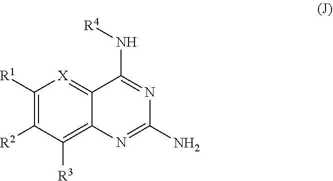

wherein

### X is N or CR¹⁰,

R¹ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups,
R² is selected from the group consisting of hydrogen, halogen, C₁₋₆alkyl, CN, —NR^{a}R^{b}, —S(O)₁₋₂R^{a} and OR^{a}, wherein C₁₋₆alkyl is optionally substituted with 1 to 5 R²⁰ groups,
R³ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups, and
R⁴ is C₁₋₁₂ alkyl which is optionally substituted with 1 to 5 substituents independently selected from halogen, —OR^{a}, —NR^{a}R^{b},
CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O) OR^{b}, —SR^{a}, —S(O)₁₋₂R^{a}, —S(O)₂NR^{a}R^{b}, —NR^{a}S(O)₂R^{b}, C₁₋₆haloalkyl, C₃₋₆cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur, and
wherein each C₃₋₆cycloalkyl, 3 to 6 membered heterocyclyl, C₆₋₁₀ aryl, and 5 to 10 membered heteroaryl is optionally substituted with 1 to 5 R²¹ groups,
R¹⁰ is selected from hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups,
each R²⁰ is independently selected from the group consisting of halogen, C₁₋₆haloalkyl, CN, —NR^{a}R^{b}, S(O)₁₋₂R^{a}, and OR^{a},
each R²¹ is independently selected from the group consisting of halogen, C₁₋₆alkyl, C₁₋₆haloalkyl, CN, —NR^{a}R^{b}, S(O)₁₋₂R^{a}, and OR^{a}, and
each R^{a} and R^{b} are independently selected from the group consisting of hydrogen and C₁₋₆alkyl, and
wherein each C₁₋₆alkyl is optionally substituted with 1 to 5 substituents independently selected from halogen, hydroxyl, amino, 5 to 10 membered heteroaryl wherein the 5 to 10 membered heteroaryl has 1 to 3 heteroatoms selected from oxygen, nitrogen, and sulfur, and C₁₋₆haloalkyl, and
provided that when X is N, R¹ is Cl, R² is H and R³ is H then R⁴ is not CH₂CH₂OMe or CH₂CH₂SO₂Me)
or pharmaceutically acceptable salts thereof;
or a pyridopyrimidine compound of formula (I)

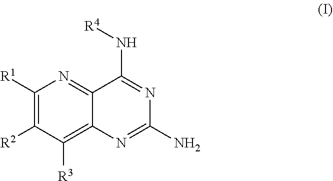

wherein
R¹ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups,
R² is selected from the group consisting of hydrogen, halogen, C₁₋₆alkyl, CN, —NR^{a}R^{b}, —S(O)₁₋₂R^{a} and OR^{a}, wherein C₁₋₆alkyl optionally substituted with 1 to 5 R²⁰ groups,
R³ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups, and
R⁴ is C₁₋₁₂ alkyl which is optionally substituted with 1 to 5 substituents independently selected from halogen, —OR^{a}, —NR^{a}R^{b},
CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆haloalkyl, C₃₋₆cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur, and
wherein each C₃₋₆cycloalkyl, 3 to 6 membered heterocyclyl, C₆₋₁₀ aryl, and 5 to 10 membered heteroaryl is optionally substituted with 1 to 5 R²¹ groups,
each R²⁰ is independently selected from the group consisting of halogen, C₁₋₆haloalkyl, CN, —NR^{a}R^{b}, S(O)₁₋₂R^{a}, and OR^{a},
each R²¹ is independently selected from the group consisting of halogen, C₁₋₆alkyl, C₁₋₆haloalkyl, CN, —NR^{a}R^{b}, S(O)₁₋₂R^{a}, and OR^{a}, and
each R^{a} and R^{b} are independently selected from the group consisting of hydrogen and C₁₋₆alkyl, wherein each C₁₋₆alkyl is optionally substituted with 1 to 5 substituents independently selected from halogen, hydroxyl, amino, 5 to 10 membered heteroaryl wherein the 5 to 10 membered heteroaryl has 1 to 3 heteroatoms selected from oxygen, nitrogen, and sulfur, and C₁₋₆haloalkyl, and
provided that when R¹ is Cl, R² is H and R³ is H then R⁴ is not CH₂CH₂OMe or CH₂CH₂SO₂Me) or pharmaceutically acceptable salts thereof.

In a particular embodiment of the application, a therapeutic combination or kit comprises: i) a first non-naturally occurring nucleic acid molecule comprising a first polynucleotide sequence encoding a truncated HBV core antigen consisting of an amino acid sequence that is at least 95% identical to SEQ ID NO: 2; ii) a second non-naturally occurring nucleic acid molecule comprising a second polynucleotide sequence encoding an HBV polymerase antigen having an amino acid sequence that is at least 90% identical to SEQ ID NO: 7, wherein the HBV polymerase antigen does not have reverse transcriptase activity and RNase H activity; and iii) any one of the following compounds:
2-amino-8-(1,4-dihydroquinazolin-2-yl)-N,N-dipropyl-3H-1-benzazepine-4-carboxamide,
2-amino-8-(1,4-dihydropyrido[3,4-d]pyrimidin-2-yl)-N,N-dipropyl-3H-1-benzazepine-4-carboxamide,
2-amino-N-(cyclopropylmethyl)-8-(1,4-dihydroquinazolin-2-yl)-N-propyl-3H-1-benzazepine-4-carboxamide,
2-amino-8-(1,4-dihydroquinazolin-2-yl)-N-isobutyl-N-propyl-3H-1-benzazepine-4-carboxamide,
2-amino-8-(5-chloro-1,4-dihydroquinazolin-2-yl)-N,N-dipropyl-3H-1-benzazepine-4-carboxamide,
2-amino-8-(7-chloro-1,4-dihydroquinazolin-2-yl)-N,N-dipropyl-3H-1-benzazepine-4-carboxamide,
2-amino-8-(4,4-dimethyl-1H-quinazolin-2-yl)-N,N-dipropyl-3H-1-benzazepine-4-carboxamide,
2-amino-8-(6-chloro-1,4-dihydroquinazolin-2-yl)-iV,iV-dipropyl-3H-1-benzazepine-4-carboxamide,
2-amino-8-(5-methyl-1,4-dihydroquinazolin-2-yl)-N,N-dipropyl-3H-1-benzazepine-4-carboxamide,
2-amino-8-(5-fluoro-1,4-dihydroquinazolin-2-yl)-N,N-dipropyl-3H-1-benzazepine-4-carboxamide,
2-amino-8-(6-methoxy-1,4-dihydroquinazolin-2-yl)-N,N-dipropyl-3H-1-benzazepine-4-carboxamide,

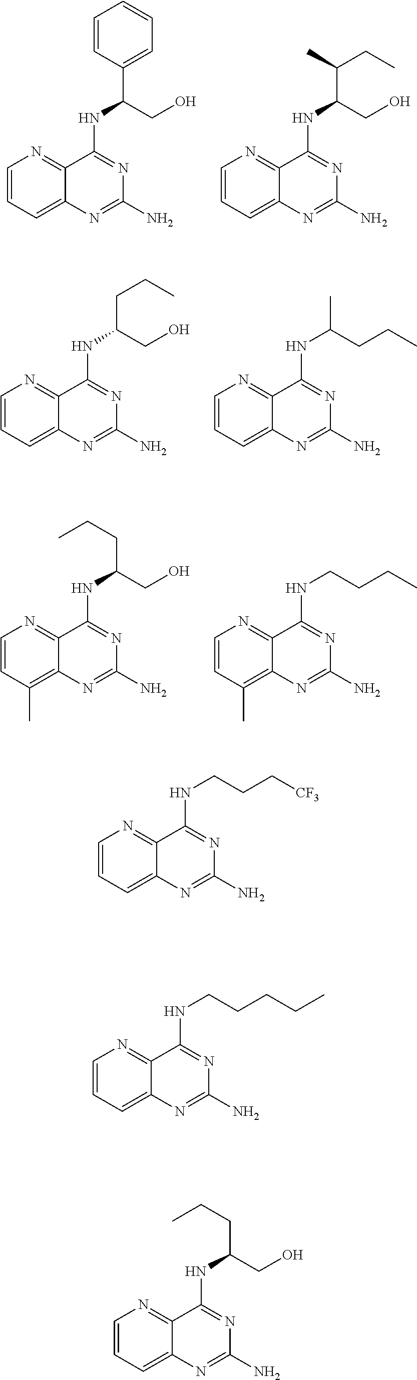

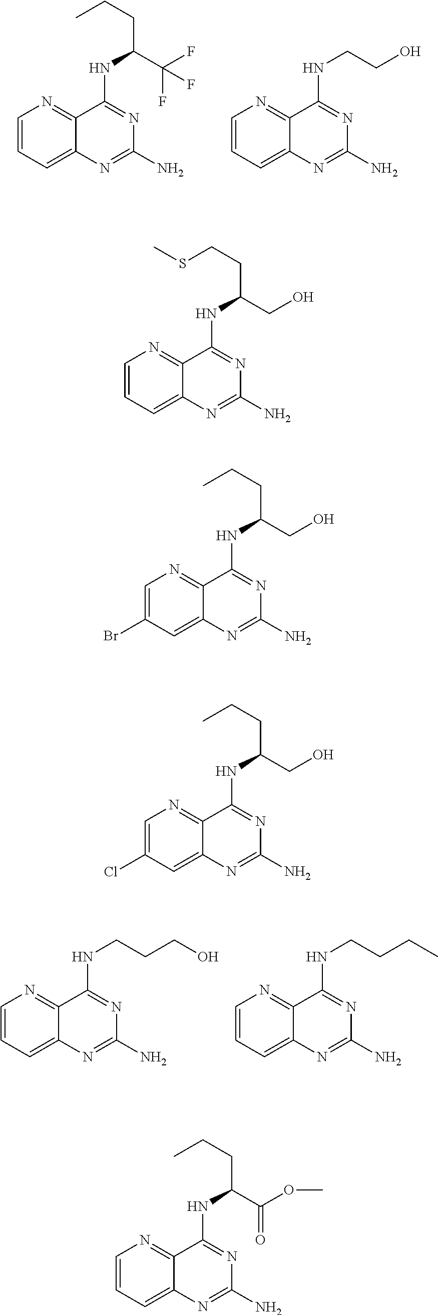

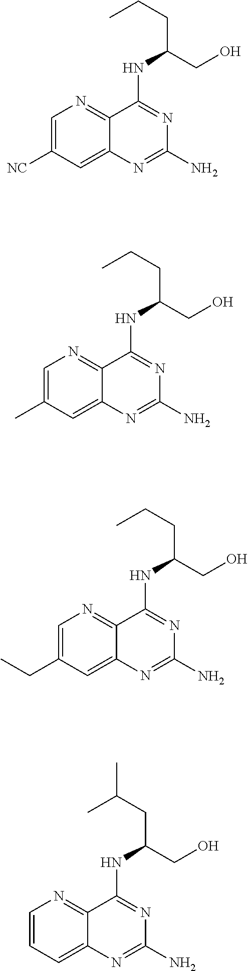 [SMILES: CCC[C@@H](CO)Nc1nc(N)nc2cc(C#N)cnc12 ; CCC[C@@H](CO)Nc1nc(N)nc2cc(C)cnc12 ; CCC[C@@H](CO)Nc1nc(N)nc2cc(CC)cnc12 ; CC(C)C[C@@H](CO)Nc1nc(N)nc2cccnc12]

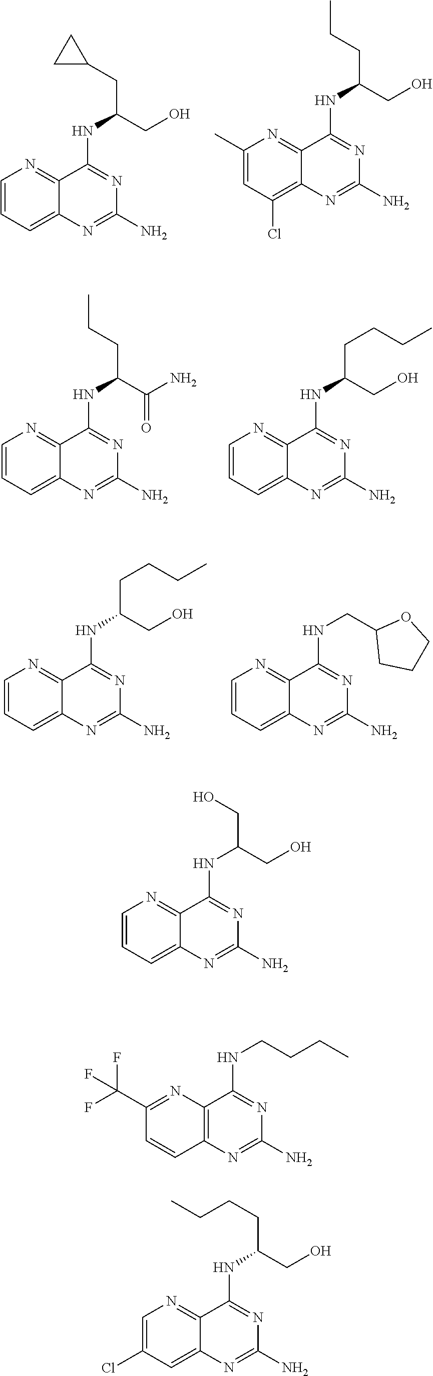

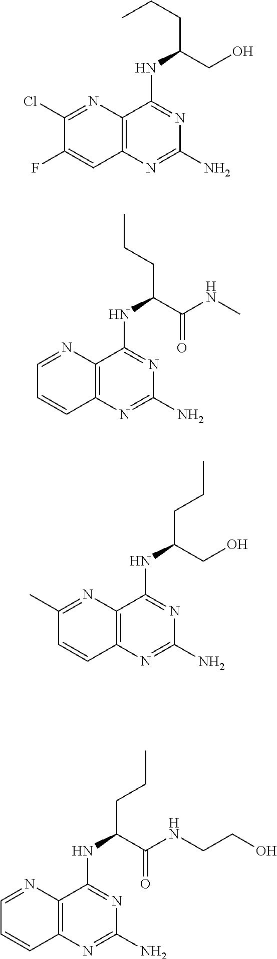

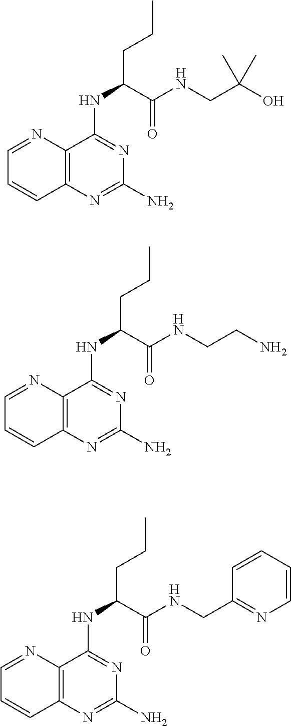

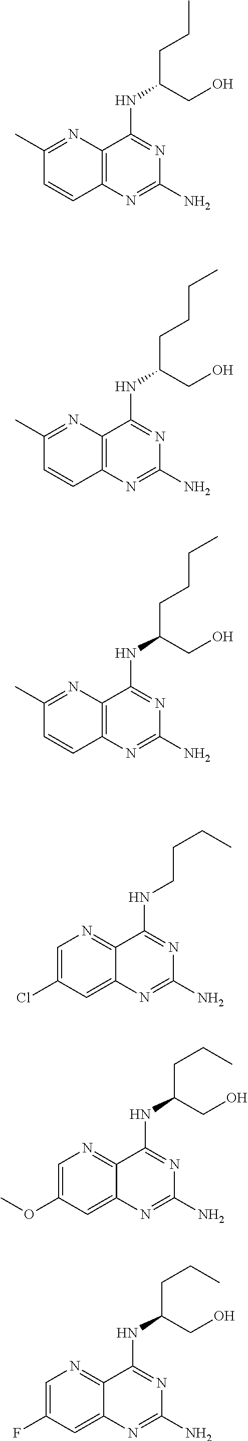

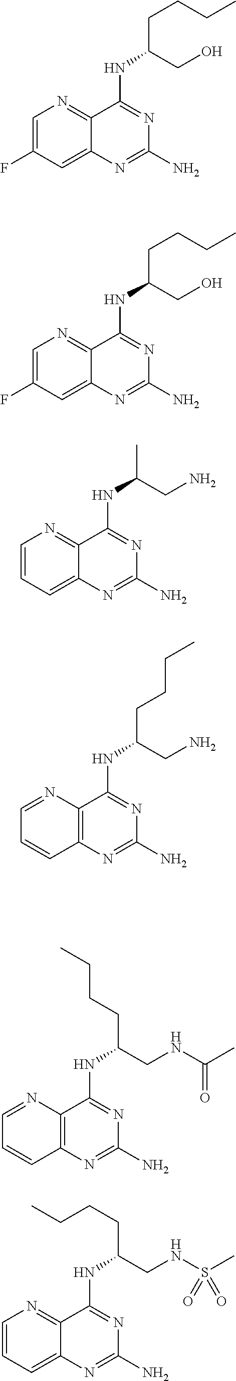

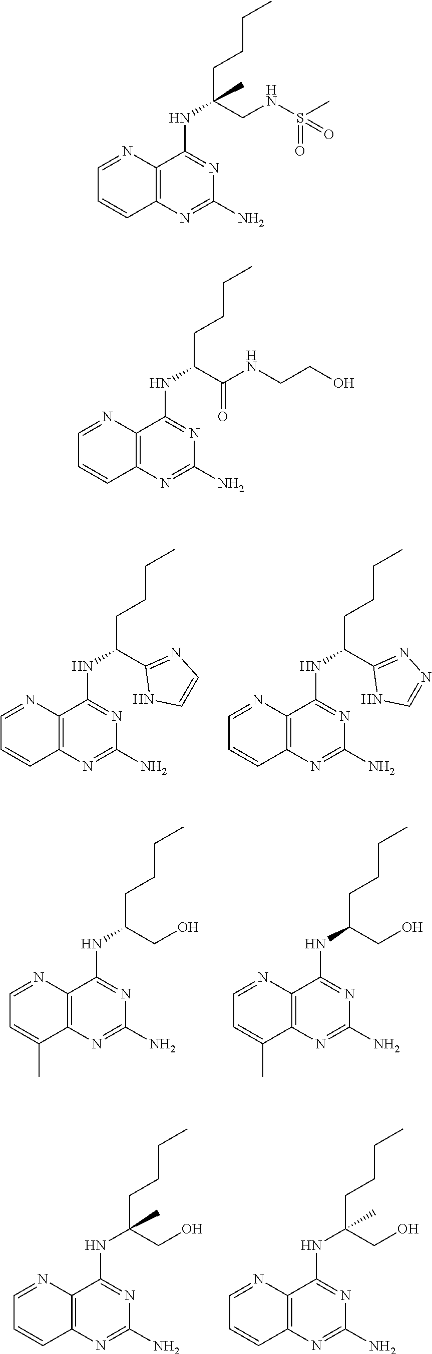

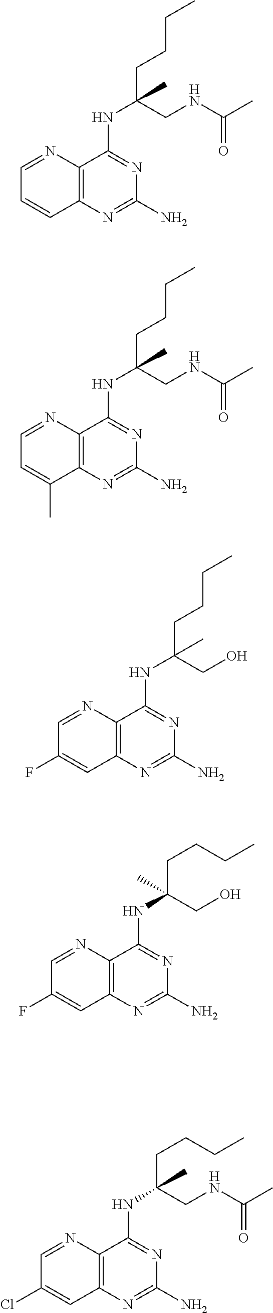

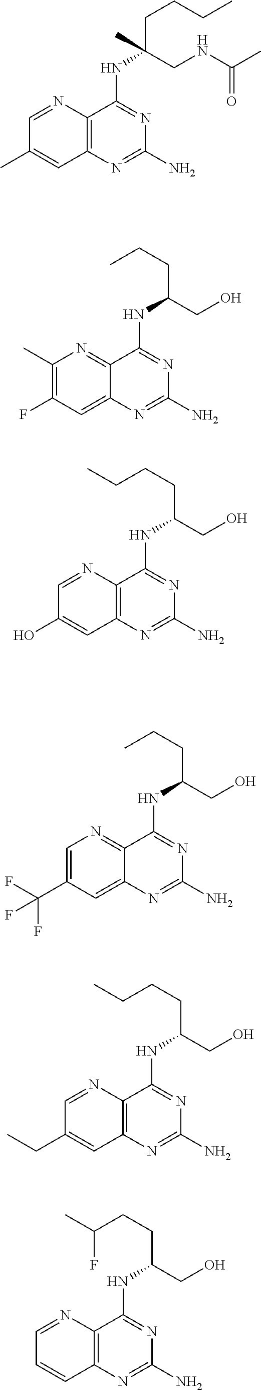

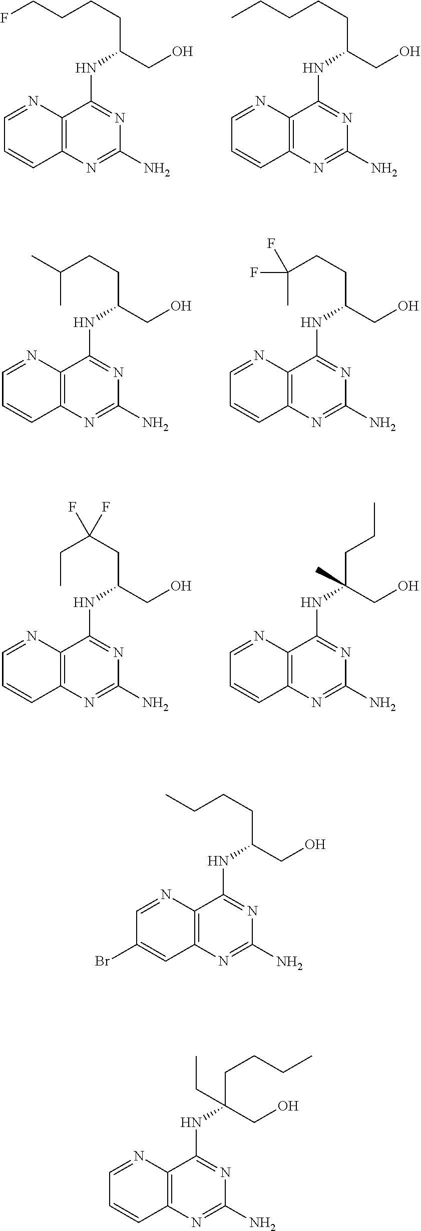

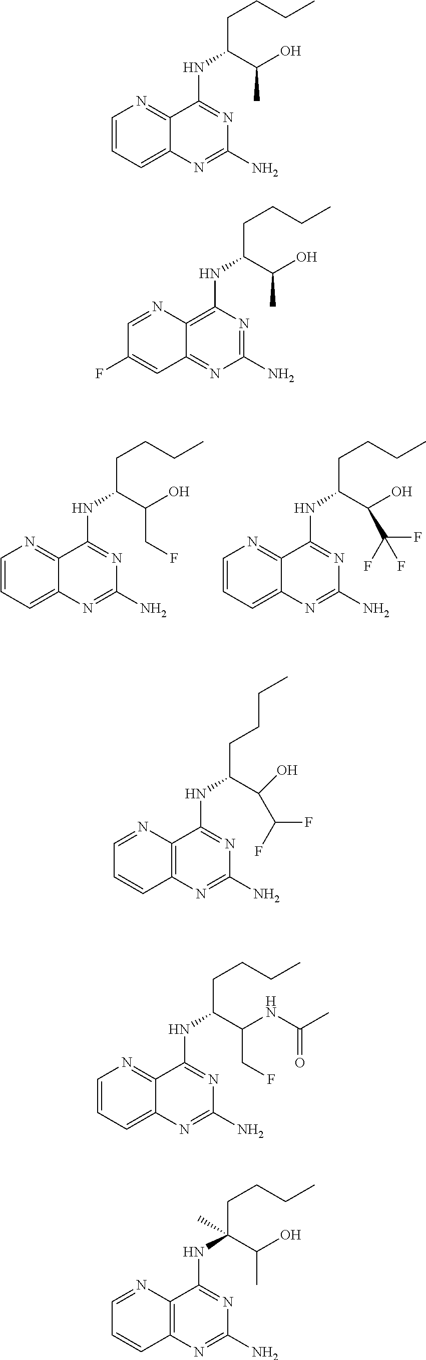

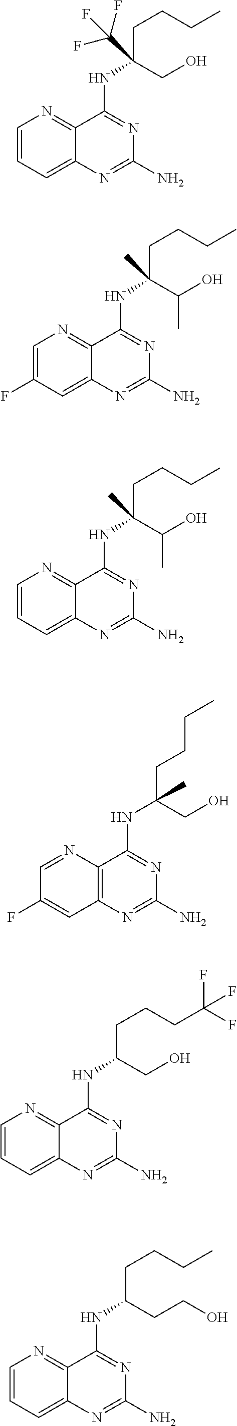 [SMILES: CCCC[C@](CO)(Nc1nc(N)nc2cccnc12)C(F)(F)F ; CCCC[C@@](C)(Nc1nc(N)nc2cc(F)cnc12)C(C)O ; CCCC[C@@](C)(Nc1nc(N)nc2cccnc12)C(C)O ; CCCC[C@](C)(CO)Nc1nc(N)nc2cc(F)cnc12 ; Nc1nc(N[C@@H](CO)CCCC(F)(F)F)c2ncccc2n1 ; CCCC[C@H](CCO)Nc1nc(N)nc2cccnc12]

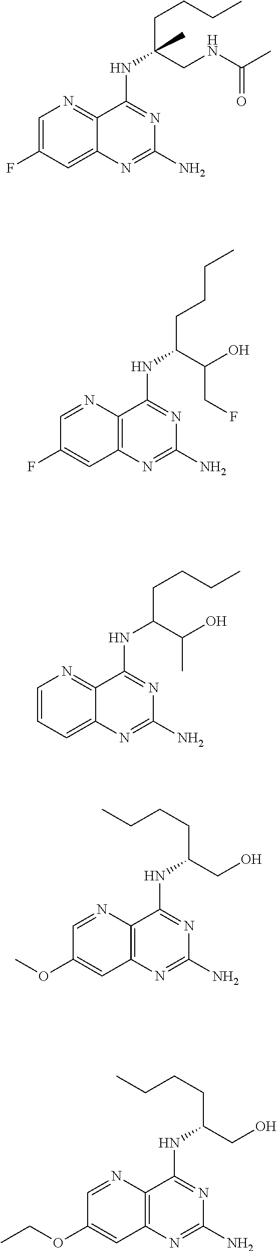

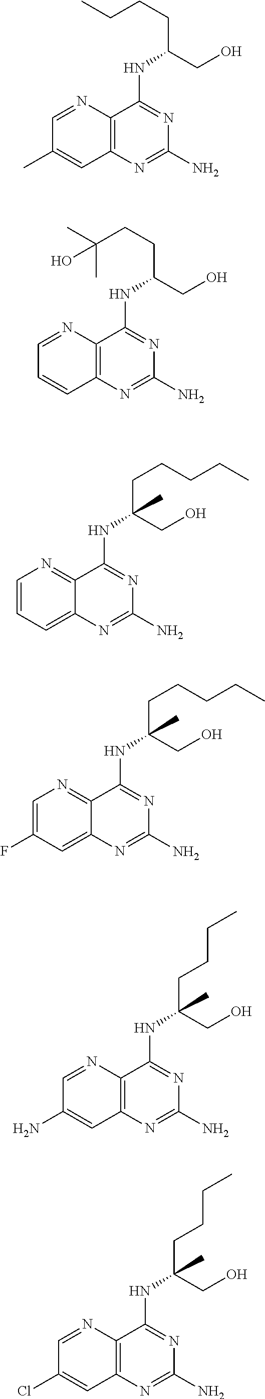

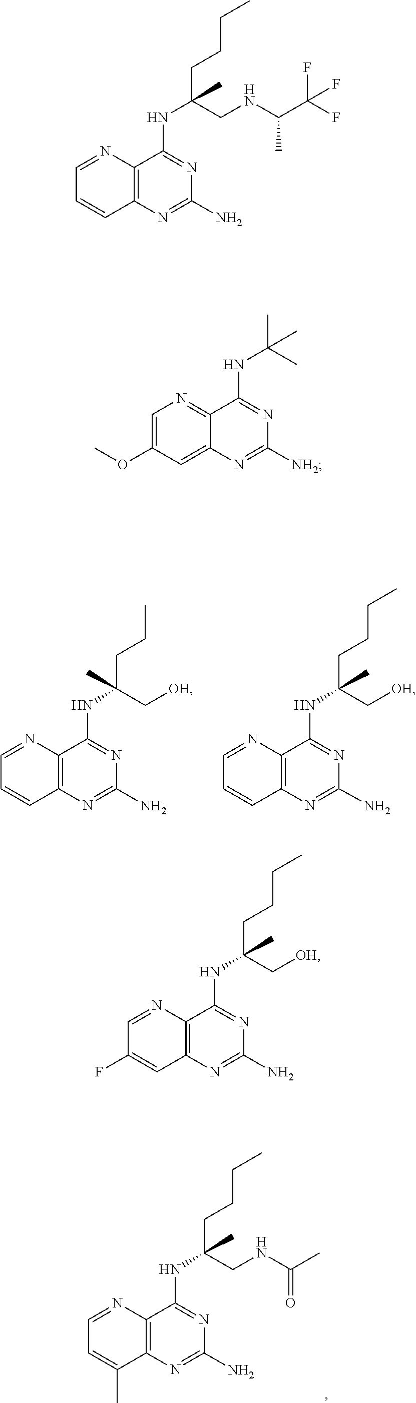

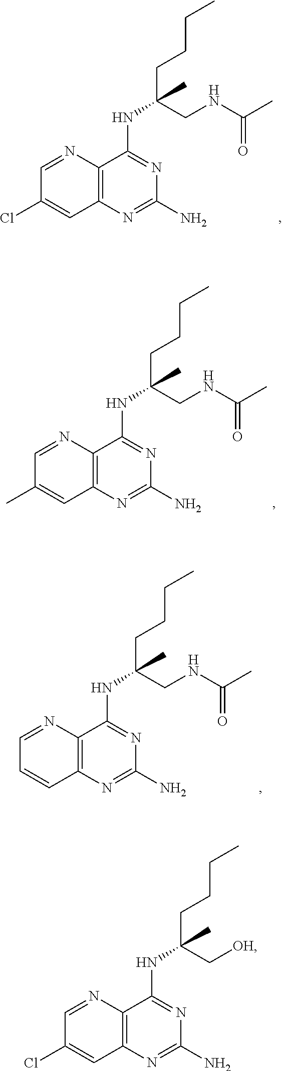

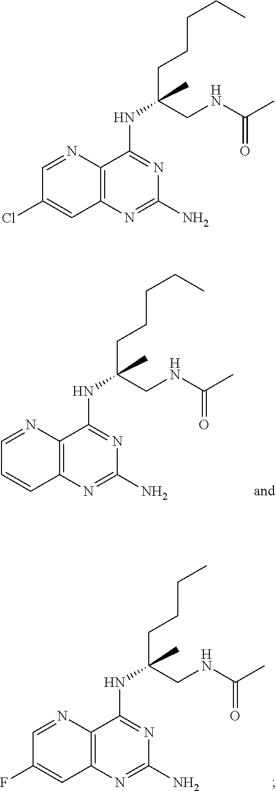

According to embodiments of the application, the polynucleotides in a vaccine combination or kit can be linked or separate, such that the HBV antigens expressed from such polynucleotides are fused together or produced as separate proteins, whether expressed from the same or different polynucleotides. In an embodiment, the first and second polynucleotides are present in separate vectors, e.g., DNA plasmids or viral vectors, used in combination either in the same or separate compositions, such that the expressed proteins are also separate proteins, but used in combination. In another embodiment, the HBV antigens encoded by the first and second polynucleotides can be expressed from the same vector, such that an HBV core-pol fusion antigen is produced. Optionally, the core and pol antigens can be joined or fused together by a short linker. Alternatively, the HBV antigens encoded by the first and second polynucleotides can be expressed independently from a single vector using a using a ribosomal slippage site (also known as cis-hydrolase site) between the core and pol antigen coding sequences. This strategy results in a bicistronic expression vector in which individual core and pol antigens are produced from a single mRNA transcript. The core and pol antigens produced from such a bicistronic expression vector can have additional N or C-terminal residues, depending upon the ordering of the coding sequences on the mRNA transcript. Examples of ribosomal slippage sites that can be used for this purpose include, but are not limited to, the FA2 slippage site from foot-and-mouth disease virus (FMDV). Another possibility is that the HBV antigens encoded by the first and second polynucleotides can be expressed independently from two separate vectors, one encoding the HBV core antigen and one encoding the HBV pol antigen.

In a preferred embodiment, the first and second polynucleotides are present in separate vectors, e.g., DNA plasmids or viral vectors. Preferably, the separate vectors are present in the same composition.

According to preferred embodiments of the application, a therapeutic combination or kit comprises a first polynucleotide present in a first vector, a second polynucleotide present in a second vector. The first and second vectors can be the same or different. Preferably the vectors are DNA plasmids.

In a particular embodiment of the application, the first vector is a first DNA plasmid, the second vector is a second DNA plasmid. Each of the first and second DNA plasmids comprises an origin of replication, preferably pUC ORI of SEQ ID NO: 21, and an antibiotic resistance cassette, preferably comprising a codon optimized Kanr gene having a polynucleotide sequence that is at least 90% identical to SEQ ID NO: 23, preferably under control of a bla promoter, for instance the bla promoter shown in SEQ ID NO: 24. Each of the first and second DNA plasmids independently further comprises at least one of a promoter sequence, enhancer sequence, and a polynucleotide sequence encoding a signal peptide sequence operably linked to the first polynucleotide sequence or the second polynucleotide sequence. Preferably, each of the first and second DNA plasmids comprises an upstream sequence operably linked to the first polynucleotide or the second polynucleotide, wherein the upstream sequence comprises, from 5′ end to 3′ end, a promoter sequence of SEQ ID NO: 18 or 19, an enhancer sequence, and a polynucleotide sequence encoding a signal peptide sequence having the amino acid sequence of SEQ ID NO: 9 or 15. Each of the first and second DNA plasmids can also comprise a polyadenylation signal located downstream of the coding sequence of the HBV antigen, such as the bGH polyadenylation signal of SEQ ID NO: 20.

In one particular embodiment of the application, the first vector is a viral vector and the second vector is a viral vector. Preferably, each of the viral vectors is an adenoviral vector, more preferably an Ad26 or Ad35 vector, comprising an expression cassette including the polynucleotide encoding an HBV pol antigen or an truncated HBV core antigen of the application; an upstream sequence operably linked to the polynucleotide encoding the HBV antigen comprising, from 5′ end to 3′ end, a promoter sequence, preferably a CMV promoter sequence of SEQ ID NO: 19, an enhancer sequence, preferably an ApoAI gene fragment sequence of SEQ ID NO: 12, and a polynucleotide sequence encoding a signal peptide sequence, preferably an immunoglobulin secretion signal having the amino acid sequence of SEQ ID NO: 15; and a downstream sequence operably linked to the polynucleotide encoding the HBV antigen comprising a polyadenylation signal, preferably a SV40 polyadenylation signal of SEQ ID NO: 13.

In another preferred embodiment, the first and second polynucleotides are present in a single vector, e.g., DNA plasmid or viral vector. Preferably, the single vector is an adenoviral vector, more preferably an Ad26 vector, comprising an expression cassette including a polynucleotide encoding an HBV pol antigen and a truncated HBV core antigen of the application, preferably encoding an HBV pol antigen and a truncated HBV core antigen of the application as a fusion protein; an upstream sequence operably linked to the polynucleotide encoding the HBV pol and truncated core antigens comprising, from 5′ end to 3′ end, a promoter sequence, preferably a CMV promoter sequence of SEQ ID NO: 19, an enhancer sequence, preferably an ApoAI gene fragment sequence of SEQ ID NO: 12, and a polynucleotide sequence encoding a signal peptide sequence, preferably an immunoglobulin secretion signal having the amino acid sequence of SEQ ID NO: 15; and a downstream sequence operably linked to the polynucleotide encoding the HBV antigen comprising a polyadenylation signal, preferably a SV40 polyadenylation signal of SEQ ID NO: 13.

When a therapeutic combination of the application comprises a first vector, such as a DNA plasmid or viral vector, and a second vector, such as a DNA plasmid or viral vector, the amount of each of the first and second vectors is not particularly limited. For example, the first DNA plasmid and the second DNA plasmid can be present in a ratio of 10:1 to 1:10, by weight, such as 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10, by weight. Preferably, the first and second DNA plasmids are present in a ratio of 1:1, by weight. The therapeutic combination of the application can further comprise a third vector encoding a third active agent useful for treating an HBV infection.

Compositions and therapeutic combinations of the application can comprise additional polynucleotides or vectors encoding additional HBV antigens and/or additional HBV antigens or immunogenic fragments thereof, such as an HBsAg, an HBV L protein or HBV envelope protein, or a polynucleotide sequence encoding thereof. However, in particular embodiments, the compositions and therapeutic combinations of the application do not comprise certain antigens.

In a particular embodiment, a composition or therapeutic combination or kit of the application does not comprise a HBsAg or a polynucleotide sequence encoding the HBsAg.

In another particular embodiment, a composition or therapeutic combination or kit of the application does not comprise an HBV L protein or a polynucleotide sequence encoding the HBV L protein.

In yet another particular embodiment of the application, a composition or therapeutic combination of the application does not comprise an HBV envelope protein or a polynucleotide sequence encoding the HBV envelope protein.

Compositions and therapeutic combinations of the application can also comprise a pharmaceutically acceptable carrier. A pharmaceutically acceptable carrier is non-toxic and should not interfere with the efficacy of the active ingredient. Pharmaceutically acceptable carriers can include one or more excipients such as binders, disintegrants, swelling agents, suspending agents, emulsifying agents, wetting agents, lubricants, flavorants, sweeteners, preservatives, dyes, solubilizers and coatings. Pharmaceutically acceptable carriers can include vehicles, such as lipid nanoparticles (LNPs). The precise nature of the carrier or other material can depend on the route of administration, e.g., intramuscular, intradermal, subcutaneous, oral, intravenous, cutaneous, intramucosal (e.g., gut), intranasal or intraperitoneal routes. For liquid injectable preparations, for example, suspensions and solutions, suitable carriers and additives include water, glycols, oils, alcohols, preservatives, coloring agents and the like. For solid oral preparations, for example, powders, capsules, caplets, gelcaps and tablets, suitable carriers and additives include starches, sugars, diluents, granulating agents, lubricants, binders, disintegrating agents and the like. For nasal sprays/inhalant mixtures, the aqueous solution/suspension can comprise water, glycols, oils, emollients, stabilizers, wetting agents, preservatives, aromatics, flavors, and the like as suitable carriers and additives.

Compositions and therapeutic combinations of the application can be formulated in any matter suitable for administration to a subject to facilitate administration and improve efficacy, including, but not limited to, oral (enteral) administration and parenteral injections. The parenteral injections include intravenous injection or infusion, subcutaneous injection, intradermal injection, and intramuscular injection. Compositions of the application can also be formulated for other routes of administration including transmucosal, ocular, rectal, long acting implantation, sublingual administration, under the tongue, from oral mucosa bypassing the portal circulation, inhalation, or intranasal.

In a preferred embodiment of the application, compositions and therapeutic combinations of the application are formulated for parental injection, preferably subcutaneous, intradermal injection, or intramuscular injection, more preferably intramuscular injection.

According to embodiments of the application, compositions and therapeutic combinations for administration will typically comprise a buffered solution in a pharmaceutically acceptable carrier, e.g., an aqueous carrier such as buffered saline and the like, e.g., phosphate buffered saline (PBS). The compositions and therapeutic combinations can also contain pharmaceutically acceptable substances as required to approximate physiological conditions such as pH adjusting and buffering agents. For example, a composition or therapeutic combination of the application comprising plasmid DNA can contain phosphate buffered saline (PBS) as the pharmaceutically acceptable carrier. The plasmid DNA can be present in a concentration of, e.g., 0.5 mg/mL to 5 mg/mL, such as 0.5 mg/mL 1, mg/mL, 2 mg/mL, 3 mg/mL, 4 mg/mL, or 5 mg/mL, preferably at 1 mg/mL.

Compositions and therapeutic combinations of the application can be formulated as a vaccine (also referred to as an “immunogenic composition”) according to methods well known in the art. Such compositions can include adjuvants to enhance immune responses. The optimal ratios of each component in the formulation can be determined by techniques well known to those skilled in the art in view of the present disclosure.

In a particular embodiment of the application, a composition or therapeutic combination is a DNA vaccine. DNA vaccines typically comprise bacterial plasmids containing a polynucleotide encoding an antigen of interest under control of a strong eukaryotic promoter. Once the plasmids are delivered to the cell cytoplasm of the host, the encoded antigen is produced and processed endogenously. The resulting antigen typically induces both humoral and cell-medicated immune responses. DNA vaccines are advantageous at least because they offer improved safety, are temperature stable, can be easily adapted to express antigenic variants, and are simple to produce. Any of the DNA plasmids of the application can be used to prepare such a DNA vaccine.

In other particular embodiments of the application, a composition or therapeutic combination is an RNA vaccine. RNA vaccines typically comprise at least one single-stranded RNA molecule encoding an antigen of interest, e.g., a fusion protein or HBV antigen according to the application. Once the RNA is delivered to the cell cytoplasm of the host, the encoded antigen is produced and processed endogenously, inducing both humoral and cell-mediated immune responses, similar to a DNA vaccine. The RNA sequence can be codon optimized to improve translation efficiency. The RNA molecule can be modified by any method known in the art in view of the present disclosure to enhance stability and/or translation, such by adding a polyA tail, e.g., of at least 30 adenosine residues; and/or capping the 5-end with a modified ribonucleotide, e.g., 7-methylguanosine cap, which can be incorporated during RNA synthesis or enzymatically engineered after RNA transcription. An RNA vaccine can also be self-replicating RNA vaccine developed from an alphavirus expression vector. Self-replicating RNA vaccines comprise a replicase RNA molecule derived from a virus belonging to the alphavirus family with a subgenomic promoter that controls replication of the fusion protein or HBV antigen RNA followed by an artificial poly A tail located downstream of the replicase.

In certain embodiments, a further adjuvant can be included in a composition or therapeutic combination of the application, or co-administered with a composition or therapeutic combination of the application. Use of another adjuvant is optional, and can further enhance immune responses when the composition is used for vaccination purposes. Other adjuvants suitable for co-administration or inclusion in compositions in accordance with the application should preferably be ones that are potentially safe, well tolerated and effective in humans. An adjuvant can be a small molecule or antibody including, but not limited to, immune checkpoint inhibitors (e.g., anti-PD1, anti-TIM-3, etc.), toll-like receptor agonists (e.g., TLR7 agonists and/or TLR8 agonists), RIG-1 agonists, IL-15 superagonists (Altor Bioscience), mutant IRF3 and IRF7 genetic adjuvants, STING agonists (Aduro), FLT3L genetic adjuvant, and IL-7-hyFc. For example, adjuvants can e.g., be chosen from among the following anti-HBV agents: HBV DNA polymerase inhibitors; Immunomodulators; Toll-like receptor 7 modulators; Toll-like receptor 8 modulators; Toll-like receptor 3 modulators; Interferon alpha receptor ligands; Hyaluronidase inhibitors; Modulators of IL-10; HBsAg inhibitors; Toll like receptor 9 modulators; Cyclophilin inhibitors; HBV Prophylactic vaccines; HBV Therapeutic vaccines; HBV viral entry inhibitors; Antisense oligonucleotides targeting viral mRNA, more particularly anti-HBV antisense oligonucleotides; short interfering RNAs (siRNA), more particularly anti-HBV siRNA; Endonuclease modulators; Inhibitors of ribonucleotide reductase; Hepatitis B virus E antigen inhibitors; HBV antibodies targeting the surface antigens of the hepatitis B virus; HBV antibodies; CCR2 chemokine antagonists; Thymosin agonists; Cytokines, such as IL12; Capsid Assembly Modulators, Nucleoprotein inhibitors (HBV core or capsid protein inhibitors); Nucleic Acid Polymers (NAPs); Stimulators of retinoic acid-inducible gene 1; Stimulators of NOD2; Recombinant thymosin alpha-1; Hepatitis B virus replication inhibitors; PI3K inhibitors; cccDNA inhibitors; immune checkpoint inhibitors, such as PD-L1 inhibitors, PD-1 inhibitors, TIM-3 inhibitors, TIGIT inhibitors, Lag3 inhibitors, CTLA-4 inhibitors; Agonists of co-stimulatory receptors that are expressed on immune cells (more particularly T cells), such as CD27 and CD28; BTK inhibitors; Other drugs for treating HBV; IDO inhibitors; Arginase inhibitors; and KDM5 inhibitors.

In certain embodiments, each of the first and second non-naturally occurring nucleic acid molecules is independently formulated with a lipid nanoparticle (LNP).

The application also provides methods of making compositions and therapeutic combinations of the application. A method of producing a composition or therapeutic combination comprises mixing an isolated polynucleotide encoding an HBV antigen, vector, and/or polypeptide of the application with one or more pharmaceutically acceptable carriers. One of ordinary skill in the art will be familiar with conventional techniques used to prepare such compositions.

### Methods of Inducing an Immune Response or Treating an HBV Infection

The application also provides methods of inducing an immune response against hepatitis B virus (HBV) in a subject in need thereof, comprising administering to the subject an immunogenically effective amount of a composition or immunogenic composition of the application. Any of the compositions and therapeutic combinations of the application described herein can be used in the methods of the application.

As used herein, the term “infection” refers to the invasion of a host by a disease causing agent. A disease causing agent is considered to be “infectious” when it is capable of invading a host, and replicating or propagating within the host. Examples of infectious agents include viruses, e.g., HBV and certain species of adenovirus, prions, bacteria, fungi, protozoa and the like. “HBV infection” specifically refers to invasion of a host organism, such as cells and tissues of the host organism, by HBV.

The phrase “inducing an immune response” when used with reference to the methods described herein encompasses causing a desired immune response or effect in a subject in need thereof against an infection, e.g., an HBV infection. “Inducing an immune response” also encompasses providing a therapeutic immunity for treating against a pathogenic agent, e.g., HBV. As used herein, the term “therapeutic immunity” or “therapeutic immune response” means that the vaccinated subject is able to control an infection with the pathogenic agent against which the vaccination was done, for instance immunity against HBV infection conferred by vaccination with HBV vaccine. In an embodiment, “inducing an immune response” means producing an immunity in a subject in need thereof, e.g., to provide a therapeutic effect against a disease, such as HBV infection. In certain embodiments, “inducing an immune response” refers to causing or improving cellular immunity, e.g., T cell response, against HBV infection. In certain embodiments, “inducing an immune response” refers to causing or improving a humoral immune response against HBV infection. In certain embodiments, “inducing an immune response” refers to causing or improving a cellular and a humoral immune response against HBV infection.

As used herein, the term “protective immunity” or “protective immune response” means that the vaccinated subject is able to control an infection with the pathogenic agent against which the vaccination was done. Usually, the subject having developed a “protective immune response” develops only mild to moderate clinical symptoms or no symptoms at all. Usually, a subject having a “protective immune response” or “protective immunity” against a certain agent will not die as a result of the infection with said agent.

Typically, the administration of compositions and therapeutic combinations of the application will have a therapeutic aim to generate an immune response against HBV after HBV infection or development of symptoms characteristic of HBV infection, e.g., for therapeutic vaccination.

As used herein, “an immunogenically effective amount” or “immunologically effective amount” means an amount of a composition, polynucleotide, vector, or antigen sufficient to induce a desired immune effect or immune response in a subject in need thereof. An immunogenically effective amount can be an amount sufficient to induce an immune response in a subject in need thereof. An immunogenically effective amount can be an amount sufficient to produce immunity in a subject in need thereof, e.g., provide a therapeutic effect against a disease such as HBV infection. An immunogenically effective amount can vary depending upon a variety of factors, such as the physical condition of the subject, age, weight, health, etc.; the particular application, e.g., providing protective immunity or therapeutic immunity; and the particular disease, e.g., viral infection, for which immunity is desired. An immunogenically effective amount can readily be determined by one of ordinary skill in the art in view of the present disclosure.

In particular embodiments of the application, an immunogenically effective amount refers to the amount of a composition or therapeutic combination which is sufficient to achieve one, two, three, four, or more of the following effects: (i) reduce or ameliorate the severity of an HBV infection or a symptom associated therewith; (ii) reduce the duration of an HBV infection or symptom associated therewith; (iii) prevent the progression of an HBV infection or symptom associated therewith; (iv) cause regression of an HBV infection or symptom associated therewith; (v) prevent the development or onset of an HBV infection, or symptom associated therewith; (vi) prevent the recurrence of an HBV infection or symptom associated therewith; (vii) reduce hospitalization of a subject having an HBV infection; (viii) reduce hospitalization length of a subject having an HBV infection; (ix) increase the survival of a subject with an HBV infection; (x) eliminate an HBV infection in a subject; (xi) inhibit or reduce HBV replication in a subject; and/or (xii) enhance or improve the prophylactic or therapeutic effect(s) of another therapy.

An immunogenically effective amount can also be an amount sufficient to reduce HBsAg levels consistent with evolution to clinical seroconversion; achieve sustained HBsAg clearance associated with reduction of infected hepatocytes by a subject's immune system; induce HBV-antigen specific activated T-cell populations; and/or achieve persistent loss of HBsAg within 12 months. Examples of a target index include lower HBsAg below a threshold of 500 copies of HBsAg international units (IU) and/or higher CD8 counts.

As general guidance, an immunogenically effective amount when used with reference to a DNA plasmid can range from about 0.1 mg/mL to 10 mg/mL of DNA plasmid total, such as 0.1 mg/mL, 0.25 mg/mL, 0.5 mg/mL. 0.75 mg/mL 1 mg/mL, 1.5 mg/mL, 2 mg/mL, 3 mg/mL, 4 mg/mL, 5 mg/mL, 6 mg/mL, 7 mg/mL, 8 mg/mL, 9 mg/mL, or 10 mg/mL. Preferably, an immunogenically effective amount of DNA plasmid is less than 8 mg/mL, more preferably less than 6 mg/mL, even more preferably 3-4 mg/mL. An immunogenically effective amount can be from one vector or plasmid, or from multiple vectors or plasmids. As further general guidance, an immunogenically effective amount when used with reference to a peptide can range from about 10 μg to 1 mg per administration, such as 10, 20, 50, 100, 200, 300, 400, 500, 600, 700, 800, 9000, or 1000 μg per administration. An immunogenically effective amount can be administered in a single composition, or in multiple compositions, such as 1, 2, 3, 4, 5, 6, 7, 8, 9, or 10 compositions (e.g., tablets, capsules or injectables, or any composition adapted to intradermal delivery, e.g., to intradermal delivery using an intradermal delivery patch), wherein the administration of the multiple capsules or injections collectively provides a subject with an immunogenically effective amount. For example, when two DNA plasmids are used, an immunogenically effective amount can be 3-4 mg/mL, with 1.5-2 mg/mL of each plasmid. It is also possible to administer an immunogenically effective amount to a subject, and subsequently administer another dose of an immunogenically effective amount to the same subject, in a so-called prime-boost regimen. This general concept of a prime-boost regimen is well known to the skilled person in the vaccine field. Further booster administrations can optionally be added to the regimen, as needed.

A therapeutic combination comprising two DNA plasmids, e.g., a first DNA plasmid encoding an HBV core antigen and second DNA plasmid encoding an HBV pol antigen, can be administered to a subject by mixing both plasmids and delivering the mixture to a single anatomic site. Alternatively, two separate immunizations each delivering a single expression plasmid can be performed. In such embodiments, whether both plasmids are administered in a single immunization as a mixture of in two separate immunizations, the first DNA plasmid and the second DNA plasmid can be administered in a ratio of 10:1 to 1:10, by weight, such as 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10, by weight. Preferably, the first and second DNA plasmids are administered in a ratio of 1:1, by weight.

Preferably, a subject to be treated according to the methods of the application is an HBV-infected subject, particular a subject having chronic HBV infection. Acute HBV infection is characterized by an efficient activation of the innate immune system complemented with a subsequent broad adaptive response (e.g., HBV-specific T-cells, neutralizing antibodies), which usually results in successful suppression of replication or removal of infected hepatocytes. In contrast, such responses are impaired or diminished due to high viral and antigen load, e.g., HBV envelope proteins are produced in abundance and can be released in sub-viral particles in 1,000-fold excess to infectious virus.

Chronic HBV infection is described in phases characterized by viral load, liver enzyme levels (necroinflammatory activity), HBeAg, or HBsAg load or presence of antibodies to these antigens. cccDNA levels stay relatively constant at approximately 10 to 50 copies per cell, even though viremia can vary considerably. The persistence of the cccDNA species leads to chronicity. More specifically, the phases of chronic HBV infection include: (i) the immune-tolerant phase characterized by high viral load and normal or minimally elevated liver enzymes; (ii) the immune activation HBeAg-positive phase in which lower or declining levels of viral replication with significantly elevated liver enzymes are observed; (iii) the inactive HBsAg carrier phase, which is a low replicative state with low viral loads and normal liver enzyme levels in the serum that may follow HBeAg seroconversion; and (iv) the HBeAg-negative phase in which viral replication occurs periodically (reactivation) with concomitant fluctuations in liver enzyme levels, mutations in the pre-core and/or basal core promoter are common, such that HBeAg is not produced by the infected cell.

As used herein, “chronic HBV infection” refers to a subject having the detectable presence of HBV for more than 6 months. A subject having a chronic HBV infection can be in any phase of chronic HBV infection. Chronic HBV infection is understood in accordance with its ordinary meaning in the field. Chronic HBV infection can for example be characterized by the persistence of HBsAg for 6 months or more after acute HBV infection. For example, a chronic HBV infection referred to herein follows the definition published by the Centers for Disease Control and Prevention (CDC), according to which a chronic HBV infection can be characterized by laboratory criteria such as: (i) negative for IgM antibodies to hepatitis B core antigen (IgM anti-HBc) and positive for hepatitis B surface antigen (HBsAg), hepatitis B e antigen (HBeAg), or nucleic acid test for hepatitis B virus DNA, or (ii) positive for HBsAg or nucleic acid test for HBV DNA, or positive for HBeAg two times at least 6 months apart.

Preferably, an immunogenically effective amount refers to the amount of a composition or therapeutic combination of the application which is sufficient to treat chronic HBV infection.

In some embodiments, a subject having chronic HBV infection is undergoing nucleoside analog (NUC) treatment, and is NUC-suppressed. As used herein, “NUC-suppressed” refers to a subject having an undetectable viral level of HBV and stable alanine aminotransferase (ALT) levels for at least six months. Examples of nucleoside/nucleotide analog treatment include HBV polymerase inhibitors, such as entacavir and tenofovir. Preferably, a subject having chronic HBV infection does not have advanced hepatic fibrosis or cirrhosis. Such subject would typically have a METAVIR score of less than 3 for fibrosis and a fibroscan result of less than 9 kPa. The METAVIR score is a scoring system that is commonly used to assess the extent of inflammation and fibrosis by histopathological evaluation in a liver biopsy of patients with hepatitis B. The scoring system assigns two standardized numbers: one reflecting the degree of inflammation and one reflecting the degree of fibrosis.

It is believed that elimination or reduction of chronic HBV may allow early disease interception of severe liver disease, including virus-induced cirrhosis and hepatocellular carcinoma. Thus, the methods of the application can also be used as therapy to treat HBV-induced diseases. Examples of HBV-induced diseases include, but are not limited to cirrhosis, cancer (e.g., hepatocellular carcinoma), and fibrosis, particularly advanced fibrosis characterized by a METAVIR score of 3 or higher for fibrosis. In such embodiments, an immunogenically effective amount is an amount sufficient to achieve persistent loss of HBsAg within 12 months and significant decrease in clinical disease (e.g., cirrhosis, hepatocellular carcinoma, etc.).

Methods according to embodiments of the application further comprises administering to the subject in need thereof another immunogenic agent (such as another HBV antigen or other antigen) or another anti-HBV agent (such as a nucleoside analog or other anti-HBV agent) in combination with a composition of the application. For example, another anti-HBV agent or immunogenic agent can be a small molecule or antibody including, but not limited to, immune checkpoint inhibitors (e.g., anti-PD1, anti-TIM-3, etc.), toll-like receptor agonists (e.g., TLR7 agonists and/or or TLR8 agonists), RIG-1 agonists, IL-15 superagonists (Altor Bioscience), mutant IRF3 and IRF7 genetic adjuvants, STING agonists (Aduro), FLT3L genetic adjuvant, IL12 genetic adjuvant, IL-7-hyFc; CAR-T which bind HBV env (S-CAR cells); capsid assembly modulators; cccDNA inhibitors, HBV polymerase inhibitors (e.g., entecavir and tenofovir). The one or other anti-HBV active agents can be, for example, a small molecule, an antibody or antigen binding fragment thereof, a polypeptide, protein, or nucleic acid. The one or other anti-HBV agents can e.g., be chosen from among HBV DNA polymerase inhibitors; Immunomodulators; Toll-like receptor 7 modulators; Toll-like receptor 8 modulators; Toll-like receptor 3 modulators; Interferon alpha receptor ligands; Hyaluronidase inhibitors; Modulators of IL-10; HBsAg inhibitors; Toll like receptor 9 modulators; Cyclophilin inhibitors; HBV Prophylactic vaccines; HBV Therapeutic vaccines; HBV viral entry inhibitors; Antisense oligonucleotides targeting viral mRNA, more particularly anti-HBV antisense oligonucleotides; short interfering RNAs (siRNA), more particularly anti-HBV siRNA; Endonuclease modulators; Inhibitors of ribonucleotide reductase; Hepatitis B virus E antigen inhibitors; HBV antibodies targeting the surface antigens of the hepatitis B virus; HBV antibodies; CCR2 chemokine antagonists; Thymosin agonists; Cytokines, such as IL12; Capsid Assembly Modulators, Nucleoprotein inhibitors (HBV core or capsid protein inhibitors); Nucleic Acid Polymers (NAPs); Stimulators of retinoic acid-inducible gene 1; Stimulators of NOD2; Recombinant thymosin alpha-1; Hepatitis B virus replication inhibitors; PI3K inhibitors; cccDNA inhibitors; immune checkpoint inhibitors, such as PD-L1 inhibitors, PD-1 inhibitors, TIM-3 inhibitors, TIGIT inhibitors, Lag3 inhibitors, and CTLA-4 inhibitors; Agonists of co-stimulatory receptors that are expressed on immune cells (more particularly T cells), such as CD27, CD28; BTK inhibitors; Other drugs for treating HBV; IDO inhibitors; Arginase inhibitors; and KDM5 inhibitors.

### Methods of Delivery

Compositions and therapeutic combinations of the application can be administered to a subject by any method known in the art in view of the present disclosure, including, but not limited to, parenteral administration (e.g., intramuscular, subcutaneous, intravenous, or intradermal injection), oral administration, transdermal administration, and nasal administration. Preferably, compositions and therapeutic combinations are administered parenterally (e.g., by intramuscular injection or intradermal injection) or transdermally.

In some embodiments of the application in which a composition or therapeutic combination comprises one or more DNA plasmids, administration can be by injection through the skin, e.g., intramuscular or intradermal injection, preferably intramuscular injection. Intramuscular injection can be combined with electroporation, i.e., application of an electric field to facilitate delivery of the DNA plasmids to cells. As used herein, the term “electroporation” refers to the use of a transmembrane electric field pulse to induce microscopic pathways (pores) in a bio-membrane. During in vivo electroporation, electrical fields of appropriate magnitude and duration are applied to cells, inducing a transient state of enhanced cell membrane permeability, thus enabling the cellular uptake of molecules unable to cross cell membranes on their own. Creation of such pores by electroporation facilitates passage of biomolecules, such as plasmids, oligonucleotides, siRNAs, drugs, etc., from one side of a cellular membrane to the other. In vivo electroporation for the delivery of DNA vaccines has been shown to significantly increase plasmid uptake by host cells, while also leading to mild-to-moderate inflammation at the injection site. As a result, transfection efficiency and immune response are significantly improved (e.g., up to 1,000 fold and 100 fold respectively) with intradermal or intramuscular electroporation, in comparison to conventional injection.

In a typical embodiment, electroporation is combined with intramuscular injection. However, it is also possible to combine electroporation with other forms of parenteral administration, e.g., intradermal injection, subcutaneous injection, etc.

Administration of a composition, therapeutic combination or vaccine of the application via electroporation can be accomplished using electroporation devices that can be configured to deliver to a desired tissue of a mammal a pulse of energy effective to cause reversible pores to form in cell membranes. The electroporation device can include an electroporation component and an electrode assembly or handle assembly. The electroporation component can include one or more of the following components of electroporation devices: controller, current waveform generator, impedance tester, waveform logger, input element, status reporting element, communication port, memory component, power source, and power switch. Electroporation can be accomplished using an in vivo electroporation device. Examples of electroporation devices and electroporation methods that can facilitate delivery of compositions and therapeutic combinations of the application, particularly those comprising DNA plasmids, include CELLECTRA® (Inovio Pharmaceuticals, Blue Bell, Pa.), Elgen electroporator (Inovio Pharmaceuticals, Inc.) Tri-Grid™ delivery system (Ichor Medical Systems, Inc., San Diego, Calif. 92121) and those described in U.S. Pat. Nos. 7,664,545, 8,209,006, 9,452,285, 5,273,525, 6,110,161, 6,261,281, 6,958,060, and 6,939,862, 7,328,064, 6,041,252, 5,873,849, 6,278,895, 6,319,901, 6,912,417, 8,187,249, 9,364,664, 9,802,035, 6,117,660, and International Patent Application Publication WO2017172838, all of which are herein incorporated by reference in their entireties. Other examples of in vivo electroporation devices are described in International Patent Application entitled “Method and Apparatus for the Delivery of Hepatitis B Virus (HBV) Vaccines,” filed on the same day as this application with the Attorney Docket Number 688097-405WO, the contents of which are hereby incorporated by reference in their entireties. Also contemplated by the application for delivery of the compositions and therapeutic combinations of the application are use of a pulsed electric field, for instance as described in, e.g., U.S. Pat. No. 6,697,669, which is herein incorporated by reference in its entirety.

In other embodiments of the application in which a composition or therapeutic combination comprises one or more DNA plasmids, the method of administration is transdermal. Transdermal administration can be combined with epidermal skin abrasion to facilitate delivery of the DNA plasmids to cells. For example, a dermatological patch can be used for epidermal skin abrasion. Upon removal of the dermatological patch, the composition or therapeutic combination can be deposited on the abraised skin.

Methods of delivery are not limited to the above described embodiments, and any means for intracellular delivery can be used. Other methods of intracellular delivery contemplated by the methods of the application include, but are not limited to, liposome encapsulation, lipid nanoparticles (LNPs), etc.

### Adjuvants

In some embodiments of the application, a method of inducing an immune response against HBV further comprises administering an adjuvant. The terms “adjuvant” and “immune stimulant” are used interchangeably herein, and are defined as one or more substances that cause stimulation of the immune system. In this context, an adjuvant is used to enhance an immune response to HBV antigens and antigenic HBV polypeptides of the application.

According to embodiments of the application, an adjuvant can be present in a therapeutic combination or composition of the application, or administered in a separate composition. An adjuvant can be, e.g., a small molecule or an antibody. Examples of adjuvants suitable for use in the application include, but are not limited to, immune checkpoint inhibitors (e.g., anti-PD1, anti-TIM-3, etc.), toll-like receptor agonists (e.g., TLR7 and/or TLR8 agonists), RIG-1 agonists, IL-15 superagonists (Altor Bioscience), mutant IRF3 and IRF7 genetic adjuvants, STING agonists (Aduro), FLT3L genetic adjuvant, IL12 genetic adjuvant, and IL-7-hyFc. Examples of adjuvants can e.g., be chosen from among the following anti-HBV agents: HBV DNA polymerase inhibitors; Immunomodulators; Toll-like receptor 7 modulators; Toll-like receptor 8 modulators; Toll-like receptor 3 modulators; Interferon alpha receptor ligands; Hyaluronidase inhibitors; Modulators of IL-10; HBsAg inhibitors; Toll like receptor 9 modulators; Cyclophilin inhibitors; HBV Prophylactic vaccines; HBV Therapeutic vaccines; HBV viral entry inhibitors; Antisense oligonucleotides targeting viral mRNA, more particularly anti-HBV antisense oligonucleotides; short interfering RNAs (siRNA), more particularly anti-HBV siRNA; Endonuclease modulators; Inhibitors of ribonucleotide reductase; Hepatitis B virus E antigen inhibitors; HBV antibodies targeting the surface antigens of the hepatitis B virus; HBV antibodies; CCR2 chemokine antagonists; Thymosin agonists; Cytokines, such as IL12; Capsid Assembly Modulators, Nucleoprotein inhibitors (HBV core or capsid protein inhibitors); Nucleic Acid Polymers (NAPs); Stimulators of retinoic acid-inducible gene 1; Stimulators of NOD2; Recombinant thymosin alpha-1; Hepatitis B virus replication inhibitors; PI3K inhibitors; cccDNA inhibitors; immune checkpoint inhibitors, such as PD-L1 inhibitors, PD-1 inhibitors, TIM-3 inhibitors, TIGIT inhibitors, Lag3 inhibitors, and CTLA-4 inhibitors; Agonists of co-stimulatory receptors that are expressed on immune cells (more particularly T cells), such as CD27, CD28; BTK inhibitors; Other drugs for treating HBV; IDO inhibitors; Arginase inhibitors; and KDM5 inhibitors.

Compositions and therapeutic combinations of the application can also be administered in combination with at least one other anti-HBV agent. Examples of anti-HBV agents suitable for use with the application include, but are not limited to small molecules, antibodies, and/or CAR-T therapies which bind HBV env (S-CAR cells), capsid assembly modulators, TLR agonists (e.g., TLR7 and/or TLR8 agonists), cccDNA inhibitors, HBV polymerase inhibitors (e.g., entecavir and tenofovir), and/or immune checkpoint inhibitors, etc.

The at least one anti-HBV agent can e.g., be chosen from among HBV DNA polymerase inhibitors; Immunomodulators; Toll-like receptor 7 modulators; Toll-like receptor 8 modulators; Toll-like receptor 3 modulators; Interferon alpha receptor ligands; Hyaluronidase inhibitors; Modulators of IL-10; HBsAg inhibitors; Toll like receptor 9 modulators; Cyclophilin inhibitors; HBV Prophylactic vaccines; HBV Therapeutic vaccines; HBV viral entry inhibitors; Antisense oligonucleotides targeting viral mRNA, more particularly anti-HBV antisense oligonucleotides; short interfering RNAs (siRNA), more particularly anti-HBV siRNA; Endonuclease modulators; Inhibitors of ribonucleotide reductase; Hepatitis B virus E antigen inhibitors; HBV antibodies targeting the surface antigens of the hepatitis B virus; HBV antibodies; CCR2 chemokine antagonists; Thymosin agonists; Cytokines, such as IL12; Capsid Assembly Modulators, Nucleoprotein inhibitors (HBV core or capsid protein inhibitors); Nucleic Acid Polymers (NAPs); Stimulators of retinoic acid-inducible gene 1; Stimulators of NOD2; Recombinant thymosin alpha-1; Hepatitis B virus replication inhibitors; PI3K inhibitors; cccDNA inhibitors; immune checkpoint inhibitors, such as PD-L1 inhibitors, PD-1 inhibitors, TIM-3 inhibitors, TIGIT inhibitors, Lag3 inhibitors, and CTLA-4 inhibitors; Agonists of co-stimulatory receptors that are expressed on immune cells (more particularly T cells), such as CD27, CD28; BTK inhibitors; Other drugs for treating HBV; IDO inhibitors; Arginase inhibitors; and KDM5 inhibitors. Such anti-HBV agents can be administered with the compositions and therapeutic combinations of the application simultaneously or sequentially.

### Methods of Prime/Boost Immunization

Embodiments of the application also contemplate administering an immunogenically effective amount of a composition or therapeutic combination to a subject, and subsequently administering another dose of an immunogenically effective amount of a composition or therapeutic combination to the same subject, in a so-called prime-boost regimen Thus, in an embodiment, a composition or therapeutic combination of the application is a primer vaccine used for priming an immune response. In another embodiment, a composition or therapeutic combination of the application is a booster vaccine used for boosting an immune response. The priming and boosting vaccines of the application can be used in the methods of the application described herein. This general concept of a prime-boost regimen is well known to the skilled person in the vaccine field. Any of the compositions and therapeutic combinations of the application described herein can be used as priming and/or boosting vaccines for priming and/or boosting an immune response against HBV.

In some embodiments of the application, a composition or therapeutic combination of the application can be administered for priming immunization. The composition or therapeutic combination can be re-administered for boosting immunization. Further booster administrations of the composition or vaccine combination can optionally be added to the regimen, as needed. An adjuvant can be present in a composition of the application used for boosting immunization, present in a separate composition to be administered together with the composition or therapeutic combination of the application for the boosting immunization, or administered on its own as the boosting immunization. In those embodiments in which an adjuvant is included in the regimen, the adjuvant is preferably used for boosting immunization.

An illustrative and non-limiting example of a prime-boost regimen includes administering a single dose of an immunogenically effective amount of a composition or therapeutic combination of the application to a subject to prime the immune response; and subsequently administering another dose of an immunogenically effective amount of a composition or therapeutic combination of the application to boost the immune response, wherein the boosting immunization is first administered about two to six weeks, preferably four weeks after the priming immunization is initially administered. Optionally, about 10 to 14 weeks, preferably 12 weeks, after the priming immunization is initially administered, a further boosting immunization of the composition or therapeutic combination, or other adjuvant, is administered.

### Kits

Also provided herein is a kit comprising a therapeutic combination of the application. A kit can comprise the first polynucleotide, the second polynucleotide, and the pyridopyrimidine derivative in one or more separate compositions, or a kit can comprise the first polynucleotide, the second polynucleotide, and the pyridopyrimidine derivative in a single composition. A kit can further comprise one or more adjuvants or immune stimulants, and/or other anti-HBV agents.

The ability to induce or stimulate an anti-HBV immune response upon administration in an animal or human organism can be evaluated either in vitro or in vivo using a variety of assays which are standard in the art. For a general description of techniques available to evaluate the onset and activation of an immune response, see for example Coligan et al. (1992 and 1994, Current Protocols in Immunology; ed. J Wiley & Sons Inc, National Institute of Health). Measurement of cellular immunity can be performed by measurement of cytokine profiles secreted by activated effector cells including those derived from CD4+ and CD8+ T-cells (e.g. quantification of IL-10 or IFN gamma-producing cells by ELISPOT), by determination of the activation status of immune effector cells (e.g. T cell proliferation assays by a classical [3H] thymidine uptake or flow cytometry-based assays), by assaying for antigen-specific T lymphocytes in a sensitized subject (e.g. peptide-specific lysis in a cytotoxicity assay, etc.).

The ability to stimulate a cellular and/or a humoral response can be determined by antibody binding and/or competition in binding (see for example Harlow, 1989, Antibodies, Cold Spring Harbor Press). For example, titers of antibodies produced in response to administration of a composition providing an immunogen can be measured by enzyme-linked immunosorbent assay (ELISA). The immune responses can also be measured by neutralizing antibody assay, where a neutralization of a virus is defined as the loss of infectivity through reaction/inhibition/neutralization of the virus with specific antibody. The immune response can further be measured by Antibody-Dependent Cellular Phagocytosis (ADCP) Assay.

### EMBODIMENTS

The invention provides also the following non-limiting embodiments.

Embodiment 1 is a therapeutic combination for use in treating a hepatitis B virus (HBV) infection in a subject in need thereof, comprising:
i) at least one of:
   a) a truncated HBV core antigen consisting of an amino acid sequence that is at least 95%, such as at least 95%, 96%, 97%, 98%, 99% or 100%, identical to SEQ ID NO: 2,
   b) a first non-naturally occurring nucleic acid molecule comprising a first polynucleotide sequence encoding the truncated HBV core antigen
   c) an HBV polymerase antigen having an amino acid sequence that is at least 90%, such as at least 90%, 91%, 92%, 93%, 94%, 95%, 96%, 97%, 98%, 99% or 100%, identical to SEQ ID NO: 7, wherein the HBV polymerase antigen does not have reverse transcriptase activity and RNase H activity, and
   d) a second non-naturally occurring nucleic acid molecule comprising a second polynucleotide sequence encoding the HBV polymerase antigen; and
ii) a benzazepine carboxamide compound of formula (K)

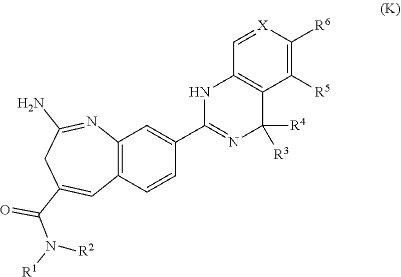

or a pharmaceutically acceptable salt thereof,

wherein R¹ is C₃₋₇-alkyl;

wherein R² is C₃₋₇-alkyl or C₃₋₇-cycloalkyl-C₁₋₇-alkyl;

wherein R³ is hydrogen or C₁₋₇-alkyl;

wherein R⁴ is hydrogen or C₁₋₇-alkyl;

wherein R⁵ is selected from the group consisting of hydrogen, halogen, C₁₋₇-alkyl and C₁₋₇-alkoxy;

wherein R⁶ is selected from the group consisting of hydrogen, halogen, C₁₋₇-alkyl and C₁₋₇-alkoxy;

wherein X is N or CR⁷,

wherein R⁷ is selected from the group consisting of hydrogen, halogen, C₁₋₇-alkyl and C₁₋₇-alkoxy.

Embodiment 1B is a therapeutic combination for use in treating a hepatitis B virus (HBV) infection in a subject in need thereof, comprising:
i) at least one of:
   a) a truncated HBV core antigen consisting of an amino acid sequence that is at least 95%, such as at least 95%, 96%, 97%, 98%, 99% or 100%, identical to SEQ ID NO: 2,
   b) a first non-naturally occurring nucleic acid molecule comprising a first polynucleotide sequence encoding the truncated HBV core antigen
   c) an HBV polymerase antigen having an amino acid sequence that is at least 90%, such as at least 90%, 91%, 92%, 93%, 94%, 95%, 96%, 97%, 98%, 99% or 100%, identical to SEQ ID NO: 7, wherein the HBV polymerase antigen does not have reverse transcriptase activity and RNase H activity, and
   d) a second non-naturally occurring nucleic acid molecule comprising a second polynucleotide sequence encoding the HBV polymerase antigen; and
      ii) a pyridopyrimidine compound of formula (J)

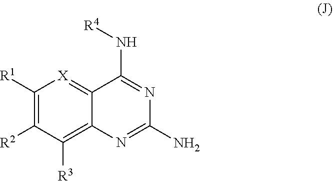

or a pharmaceutically acceptable salt thereof,
wherein X is N or CR¹⁰,

wherein R¹ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups,

wherein R² is selected from the group consisting of hydrogen, halogen, C₁₋₆alkyl, CN, —NR^{a}R^{b}, —S(O)₁₋₂R^{a} and OR^{a}, wherein C₁₋₆alkyl is optionally substituted with 1 to 5 R²⁰ groups,

wherein R³ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups,

wherein R⁴ is C₁₋₁₂ alkyl which is optionally substituted with 1 to 5 substituents independently selected from halogen, —OR^{a}, —NR^{a}R^{b},

CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O) OR^{b}, —SR^{a}, —S(O)₁₋₂R^{a}, —S(O)₂NR^{a}R^{b}, —NR^{a}S(O)₂R^{b}, C₁₋₆haloalkyl, C₃₋₆cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur,

wherein each C₃₋₆cycloalkyl, 3 to 6 membered heterocyclyl, C₆₋₁₀ aryl, and 5 to 10 membered heteroaryl is optionally substituted with 1 to 5 R²¹ groups,

wherein R¹⁰ is selected from hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups,

wherein each R²⁰ is independently selected from the group consisting of halogen, C₁₋₆haloalkyl, CN, —NR^{a}R^{b}, S(O)₁₋₂R^{a}, and OR^{a},

wherein each R²¹ is independently selected from the group consisting of halogen, C₁₋₆alkyl, C₁₋₆haloalkyl, CN, —NR^{a}R^{b}, S(O)₁₋₂R^{a}, and OR^{a}, and

wherein each R^{a} and R^{b} are independently selected from the group consisting of hydrogen and C₁₋₆alkyl,

wherein each C₁₋₆alkyl is optionally substituted with 1 to 5 substituents independently selected from halogen, hydroxyl, amino, 5 to 10 membered heteroaryl wherein the 5 to 10 membered heteroaryl has 1 to 3 heteroatoms selected from oxygen, nitrogen, and sulfur, and C₁₋₆haloalkyl,

provided that when X is N, R¹ is Cl, R² is H and R³ is H then R⁴ is not CH₂CH₂OMe or CH₂CH₂SO₂Me.

Embodiment 1C is a therapeutic combination for use in treating a hepatitis B virus (HBV) infection in a subject in need thereof, comprising:
i) at least one of:
   a) a truncated HBV core antigen consisting of an amino acid sequence that is at least 95%, such as at least 95%, 96%, 97%, 98%, 99% or 100%, identical to SEQ ID NO: 2,
   b) a first non-naturally occurring nucleic acid molecule comprising a first polynucleotide sequence encoding the truncated HBV core antigen
   c) an HBV polymerase antigen having an amino acid sequence that is at least 90%, such as at least 90%, 91%, 92%, 93%, 94%, 95%, 96%, 97%, 98%, 99% or 100%, identical to SEQ ID NO: 7, wherein the HBV polymerase antigen does not have reverse transcriptase activity and RNase H activity, and
   d) a second non-naturally occurring nucleic acid molecule comprising a second polynucleotide sequence encoding the HBV polymerase antigen; and
      ii) a pyridopyrimidine compound of formula (I)

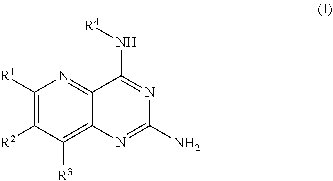

or a pharmaceutically acceptable salt thereof,

wherein R¹ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups;

wherein R² is selected from the group consisting of hydrogen, halogen, C₁₋₆alkyl, CN, —NR^{a}R^{b}, —S(O)₁₋₂R^{a} and OR^{a}, wherein C₁₋₆alkyl optionally substituted with 1 to 5 R²⁰ groups;

wherein R³ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups;

wherein R⁴ is C₁₋₁₂ alkyl which is optionally substituted with 1 to 5 substituents independently selected from halogen, —OR^{a}, —NR^{a}R^{b},

CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆haloalkyl, C₃₋₆cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur;

wherein each C₃₋₆cycloalkyl, 3 to 6 membered heterocyclyl, C₆₋₁₀ aryl, and 5 to 10 membered heteroaryl is optionally substituted with 1 to 5 R²¹ groups;

wherein each R²⁰ is independently selected from the group consisting of halogen, C₁₋₆haloalkyl, CN, —NR^{a}R^{b}, S(O)₁₋₂R^{a}, and OR^{a};

wherein each R²¹ is independently selected from the group consisting of halogen, C₁₋₆alkyl, C₁₋₆haloalkyl, CN, —NR^{a}R^{b}, S(O)₁₋₂R^{a}, and OR^{a}; and

wherein each R^{a} and R^{b} are independently selected from the group consisting of hydrogen and C₁₋₆alkyl, wherein each C₁₋₆alkyl is optionally substituted with 1 to 5 substituents independently selected from halogen, hydroxyl, amino, 5 to 10 membered heteroaryl wherein the 5 to 10 membered heteroaryl has 1 to 3 heteroatoms selected from oxygen, nitrogen, and sulfur, and C₁₋₆haloalkyl;

provided that when R¹ is Cl, R² is H and R³ is H then R⁴ is not CH₂CH₂OMe or CH₂CH₂SO₂Me.

Embodiment 2 is the therapeutic combination of any one of embodiments 1 through 1C, comprising at least one of the HBV polymerase antigen and the truncated HBV core antigen.

Embodiment 3 is the therapeutic combination of embodiment 2, comprising the HBV polymerase antigen and the truncated HBV core antigen.

Embodiment 4 is the therapeutic combination of any one of embodiments 1 through 1C, comprising at least one of the first non-naturally occurring nucleic acid molecule comprising the first polynucleotide sequence encoding the truncated HBV core antigen, and the second non-naturally occurring nucleic acid molecule comprising the second polynucleotide sequence encoding the HBV polymerase antigen.

Embodiment 5 is a therapeutic combination for use in treating a hepatitis B virus (HBV) infection in a subject in need thereof, comprising
i) a first non-naturally occurring nucleic acid molecule comprising a first polynucleotide sequence encoding a truncated HBV core antigen consisting of an amino acid sequence that is at least 95% identical to SEQ ID NO: 2; and
ii) a second non-naturally occurring nucleic acid molecule comprising a second polynucleotide sequence encoding an HBV polymerase antigen having an amino acid sequence that is at least 90% identical to SEQ ID NO: 7, wherein the HBV polymerase antigen does not have reverse transcriptase activity and RNase H activity; and
iii) a benzazepine carboxamide compound of formula (K)

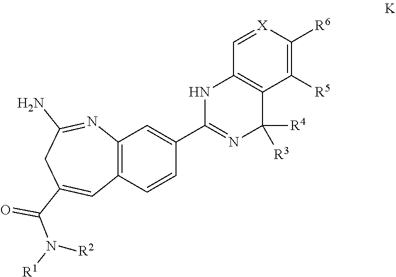

or a pharmaceutically acceptable salt thereof,
wherein R¹ is C₃₋₇-alkyl,
wherein R² is C₃₋₇-alkyl or C₃₋₇-cycloalkyl-C₁₋₇alkyl,
wherein R³ is hydrogen or C₁₋₇-alkyl,
wherein R⁴ is hydrogen or C₁₋₇-alkyl,
wherein R⁵ is selected from the group consisting of hydrogen, halogen, C₁₋₇-alkyl and C₁₋₇-alkoxy,
wherein R⁶ is selected from the group consisting of hydrogen, halogen, C₁₋₇-alkyl and C₁₋₇-alkoxy,
wherein X is N or CR⁷, and
wherein R⁷ is selected from the group consisting of hydrogen, halogen, C₁₋₇-alkyl and C₁₋₇-alkoxy.

Embodiment 5B is a therapeutic combination for use in treating a hepatitis B virus (HBV) infection in a subject in need thereof, comprising
i) a first non-naturally occurring nucleic acid molecule comprising a first polynucleotide sequence encoding a truncated HBV core antigen consisting of an amino acid sequence that is at least 95% identical to SEQ ID NO: 2; and
ii) a second non-naturally occurring nucleic acid molecule comprising a second polynucleotide sequence encoding an HBV polymerase antigen having an amino acid sequence that is at least 90% identical to SEQ ID NO: 7, wherein the HBV polymerase antigen does not have reverse transcriptase activity and RNase H activity; and
iii) a pyridopyrimidine compound of formula (J)

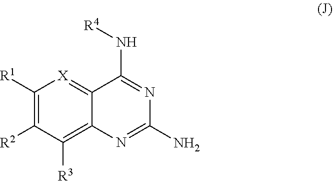

or a pharmaceutically acceptable salt thereof,
wherein X is N or CR¹⁰,
wherein R¹ is selected from the group consisting of hydrogen, halogen, C₁₋₆alkyl,
CN, —NR^{a}R^{b}, —S(O)₁₋₂R^{a}, and OR^{a}, wherein C₁₋₆alkyl is optionally substituted with 1 to 5 R²⁰ groups,
wherein R² is selected from the group consisting of hydrogen, halogen, C₁₋₆alkyl,
CN, —NR^{a}R^{b}, —S(O)₁₋₂R^{a} and OR^{a}, wherein C₁₋₆alkyl is optionally substituted with 1 to 5 R²⁰ groups,
wherein R³ is selected from the group consisting of hydrogen, halogen, C₁₋₆alkyl,
CN, —NR^{a}R^{b}, —S(O)₁₋₂R^{a}, and OR^{a}, wherein C₁₋₆alkyl is optionally substituted with 1 to 5 R²⁰ groups,
wherein R⁴ is C₁₋₁₂ alkyl which is optionally substituted with 1 to 5 substituents independently selected from halogen, —OR^{a}, —NR^{a}R^{b},
CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O) OR^{b}, —SR^{a}, —S(O)₁₋₂R^{a}, —S(O)₂NR^{a}R^{b}, —NR^{a}S(O)₂R^{b}, C₁₋₆haloalkyl, C₃₋₆cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur,
wherein each C₃₋₆cycloalkyl, 3 to 6 membered heterocyclyl, C₆₋₁₀ aryl, and 5 to 10 membered heteroaryl is optionally substituted with 1 to 5 R²¹ groups,
wherein R¹⁰ is selected from hydrogen, halogen, C₁₋₆alkyl, CN, —NR^{a}R^{b}, —S(O)₁₋₂R^{a}, and OR^{a},
wherein C₁₋₆alkyl is optionally substituted with 1 to 5 R²⁰ groups,
wherein each R²⁰ is independently selected from the group consisting of halogen, C₁₋₆haloalkyl, CN, —NR^{a}R^{b}, S(O)₁₋₂R^{a}, and OR^{a},
wherein each R²¹ is independently selected from the group consisting of halogen, C₁₋₆alkyl, C₁₋₆haloalkyl, CN, —NR^{a}R^{b}, S(O)₁₋₂R^{a}, and OR^{a},
wherein each R^{a} and R^{b} are independently selected from the group consisting of hydrogen and C₁₋₆alkyl, and
wherein each C₁₋₆alkyl is optionally substituted with 1 to 5 substituents independently selected from halogen, hydroxyl, amino, 5 to 10 membered heteroaryl wherein the 5 to 10 membered heteroaryl has 1 to 3 heteroatoms selected from oxygen, nitrogen, and sulfur, and C₁₋₆haloalkyl, and
provided that when X is N, R¹ is Cl, R² is H and R³ is H then R⁴ is not CH₂CH₂OMe or CH₂CH₂SO₂Me.

Embodiment 5C is a therapeutic combination for use in treating a hepatitis B virus (HBV) infection in a subject in need thereof, comprising
i) a first non-naturally occurring nucleic acid molecule comprising a first polynucleotide sequence encoding a truncated HBV core antigen consisting of an amino acid sequence that is at least 95% identical to SEQ ID NO: 2; and
ii) a second non-naturally occurring nucleic acid molecule comprising a second polynucleotide sequence encoding an HBV polymerase antigen having an amino acid sequence that is at least 90% identical to SEQ ID NO: 7, wherein the HBV polymerase antigen does not have reverse transcriptase activity and RNase H activity; and
iii) a pyridopyrimidine compound of formula (I)

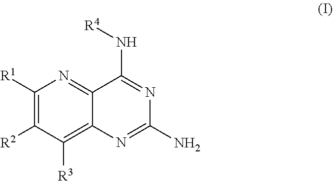

or a pharmaceutically acceptable salt thereof,
wherein R¹ is selected from the group consisting of hydrogen, halogen, C₁₋₆alkyl,
CN, —NR^{a}R^{b}, —S(O)₁₋₂R^{a}, and OR^{a}, wherein C₁₋₆alkyl is optionally substituted with 1 to 5 R²⁰ groups,
wherein R² is selected from the group consisting of hydrogen, halogen, C₁₋₆alkyl,
CN, —NR^{a}R^{b}, —S(O)₁₋₂R^{a} and OR^{a}, wherein C₁₋₆alkyl optionally substituted with 1 to 5 R²⁰ groups,
wherein R³ is selected from the group consisting of hydrogen, halogen, C₁₋₆alkyl,
CN, —NR^{a}R^{b}, —S(O)₁₋₂R^{a}, and OR^{a}, wherein C₁₋₆alkyl is optionally substituted with 1 to 5 R²⁰ groups,
wherein R⁴ is C₁₋₁₂ alkyl which is optionally substituted with 1 to 5 substituents independently selected from halogen, —OR^{a}, —NR^{a}R^{b},
CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆haloalkyl, C₃₋₆cycloalkyl, 3 to 6 membered heterocyclyl wherein the 3 to 6 membered heterocycyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur,
wherein each C₃₋₆cycloalkyl, 3 to 6 membered heterocyclyl, C₆₋₁₀ aryl, and 5 to 10 membered heteroaryl is optionally substituted with 1 to 5 R²¹ groups,
wherein each R²⁰ is independently selected from the group consisting of halogen, C₁₋₆haloalkyl, CN, —NR^{a}R^{b}, S(O)₁₋₂R^{a}, and OR^{a},
wherein each R²¹ is independently selected from the group consisting of halogen, C₁₋₆alkyl, C₁₋₆haloalkyl, CN, —NR^{a}R^{b}, S(O)₁₋₂R^{a}, and OR^{a}, and
wherein each R^{a} and R^{b} are independently selected from the group consisting of hydrogen and C₁₋₆alkyl, wherein each C₁₋₆alkyl is optionally substituted with 1 to 5 substituents independently selected from halogen, hydroxyl, amino, 5 to 10 membered heteroaryl wherein the 5 to 10 membered heteroaryl has 1 to 3 heteroatoms selected from oxygen, nitrogen, and sulfur, and C₁₋₆haloalkyl, and
provided that when R¹ is Cl, R² is H and R³ is H then R⁴ is not CH₂CH₂OMe or CH₂CH₂SO₂Me.

Embodiment 6 is the therapeutic combination of embodiment 4 or 5, wherein the first non-naturally occurring nucleic acid molecule further comprises a polynucleotide sequence encoding a signal sequence operably linked to the N-terminus of the truncated HBV core antigen.

Embodiment 6a is the therapeutic combination of any one of embodiments 4 to 6, wherein the second non-naturally occurring nucleic acid molecule further comprises a polynucleotide sequence encoding a signal sequence operably linked to the N-terminus of the HBV polymerase antigen.

Embodiment 6b is the therapeutic combination of embodiment 6 or 6a, wherein the signal sequence independently comprises the amino acid sequence of SEQ ID NO: 9 or SEQ ID NO: 15.

Embodiment 6c is the therapeutic combination of embodiment 6 or 6a, wherein the signal sequence is independently encoded by the polynucleotide sequence of SEQ ID NO: 8 or SEQ ID NO: 14.

Embodiment 7 is the therapeutic combination of any one of embodiments 1-6c, wherein the HBV polymerase antigen comprises an amino acid sequence that is at least 98%, such as at least 98%, 98.5%, 99%, 99.1%, 99.2%, 99.3%, 99.4%, 99.5%, 99.6%, 99.7%, 99.8%, 99.9%, or 100%, identical to SEQ ID NO: 7.

Embodiment 7a is the therapeutic combination of embodiment 7, wherein the HBV polymerase antigen comprises the amino acid sequence of SEQ ID NO: 7.

Embodiment 7b is the therapeutic combination of any one of embodiments 1 to 7a, wherein and the truncated HBV core antigen consists of the amino acid sequence that is at least 98%, such as at least 98%, 98.5%, 99%, 99.1%, 99.2%, 99.3%, 99.4%, 99.5%, 99.6%, 99.7%, 99.8%, 99.9%, or 100%, identical to SEQ ID NO: 2.

Embodiment 7c is the therapeutic combination of embodiment 7b, wherein the truncated HBV antigen consists of the amino acid sequence of SEQ ID NO: 2 or SEQ ID NO: 4.

Embodiment 8 is the therapeutic combination of any one of embodiments 1-7c, wherein each of the first and second non-naturally occurring nucleic acid molecules is a DNA molecule.

Embodiment 8a is the therapeutic combination of embodiment 8, wherein the DNA molecule is present on a DNA vector.

Embodiment 8b is the therapeutic combination of embodiment 8a, wherein the DNA vector is selected from the group consisting of DNA plasmids, bacterial artificial chromosomes, yeast artificial chromosomes, and closed linear deoxyribonucleic acid.

Embodiment 8c is the therapeutic combination of embodiment 8, wherein the DNA molecule is present on a viral vector.

Embodiment 8d is the therapeutic combination of embodiment 8c, wherein the viral vector is selected from the group consisting of bacteriophages, animal viruses, and plant viruses.

Embodiment 8e is the therapeutic combination of any one of embodiments 1-7c, wherein each of the first and second non-naturally occurring nucleic acid molecules is an RNA molecule.

Embodiment 8f is the therapeutic combination of embodiment 8e, wherein the RNA molecule is an RNA replicon, preferably a self-replicating RNA replicon, an mRNA replicon, a modified mRNA replicon, or self-amplifying mRNA.

Embodiment 8g is the therapeutic combination of any one of embodiments 1 to 8f, wherein each of the first and second non-naturally occurring nucleic acid molecules is independently formulated with a lipid composition, preferably a lipid nanoparticle (LNP).

Embodiment 9 is the therapeutic combination of any one of embodiments 4-8g, comprising the first non-naturally occurring nucleic acid molecule and the second non-naturally occurring nucleic acid molecule in the same non-naturally occurring nucleic acid molecule.

Embodiment 10 is the therapeutic combination of any one of embodiments 4-8g, comprising the first non-naturally occurring nucleic acid molecule and the second non-naturally occurring nucleic acid molecule in two different non-naturally occurring nucleic acid molecules.

Embodiment 11 is the therapeutic combination of any one of embodiments 4-10, wherein the first polynucleotide sequence comprises a polynucleotide sequence having at least 90%, such as at least 90%, 91%, 92%, 93%, 94%, 95%, 96%, 97%, 98%, 99% or 100%, sequence identity to SEQ ID NO: 1 or SEQ ID NO: 3.

Embodiment 11a is the therapeutic combination of embodiment 11, wherein the first polynucleotide sequence comprises a polynucleotide sequence having at least 98%, such as at least 98%, 98.5%, 99%, 99.1%, 99.2%, 99.3%, 99.4%, 99.5%, 99.6%, 99.7%, 99.8%, 99.9%, or 100%, sequence identity to SEQ ID NO: 1 or SEQ ID NO: 3.

Embodiment 12 is the therapeutic combination of embodiment 11a, wherein the first polynucleotide sequence comprises the polynucleotide sequence of SEQ ID NO: 1 or SEQ ID NO: 3.

Embodiment 13 the therapeutic combination of any one of embodiments 4 to 12, wherein the second polynucleotide sequence comprises a polynucleotide sequence having at least 90%, such as at least 90%, 91%, 92%, 93%, 94%, 95%, 96%, 97%, 98%, 99% or 100%, sequence identity to SEQ ID NO: 5 or SEQ ID NO: 6.

Embodiment 13a the therapeutic combination of embodiment 13, wherein the second polynucleotide sequence comprises a polynucleotide sequence having at least 98%, such as at least 98%, 98.5%, 99%, 99.1%, 99.2%, 99.3%, 99.4%, 99.5%, 99.6%, 99.7%, 99.8%, 99.9%, or 100%, sequence identity to SEQ ID NO: 5 or SEQ ID NO: 6.

Embodiment 14 is the therapeutic combination of embodiment 13a, wherein the second polynucleotide sequence comprises the polynucleotide sequence of SEQ ID NO: 5 or SEQ ID NO: 6.

Embodiment 15 is the therapeutic combination of any one of embodiments 1 through 14, wherein the compound is selected from the group consisting of
2-amino-8-(1,4-dihydroquinazolin-2-yl)-N,N-dipropyl-3H-1-benzazepine-4-carboxamide,
2-amino-8-(1,4-dihydropyrido[3,4-d]pyrimidin-2-yl)-N,N-dipropyl-3H-1-benzazepine-4-carboxamide,
2-amino-N-(cyclopropylmethyl)-8-(1,4-dihydroquinazolin-2-yl)-N-propyl-3H-1-benzazepine-4-carboxamide,
2-amino-8-(1,4-dihydroquinazolin-2-yl)-N-isobutyl-N-propyl-3H-1-benzazepine-4-carboxamide,
2-amino-8-(5-chloro-1,4-dihydroquinazolin-2-yl)-N,N-dipropyl-3H-1-benzazepine-4-carboxamide,
2-amino-8-(7-chloro-1,4-dihydroquinazolin-2-yl)-N,N-dipropyl-3H-1-benzazepine-4-carboxamide,
2-amino-8-(4,4-dimethyl-1H-quinazolin-2-yl)-N,N-dipropyl-3H-1-benzazepine-4-carboxamide,
2-amino-8-(6-chloro-1,4-dihydroquinazolin-2-yl)-iV,iV-dipropyl-3H-1-benzazepine-4-carboxamide,
2-amino-8-(5-methyl-1,4-dihydroquinazolin-2-yl)-N,N-dipropyl-3H-1-benzazepine-4-carboxamide,
2-amino-8-(5-fluoro-1,4-dihydroquinazolin-2-yl)-N,N-dipropyl-3H-1-benzazepine-4-carboxamide, and
2-amino-8-(6-methoxy-1,4-dihydroquinazolin-2-yl)-N,N-dipropyl-3H-1-benzazepine-4-carboxamide,

or a pharmaceutically acceptable salt thereof.

Embodiment 15B. The therapeutic combination of any one of claims 1 through 14, wherein the compound is selected from the group consisting of

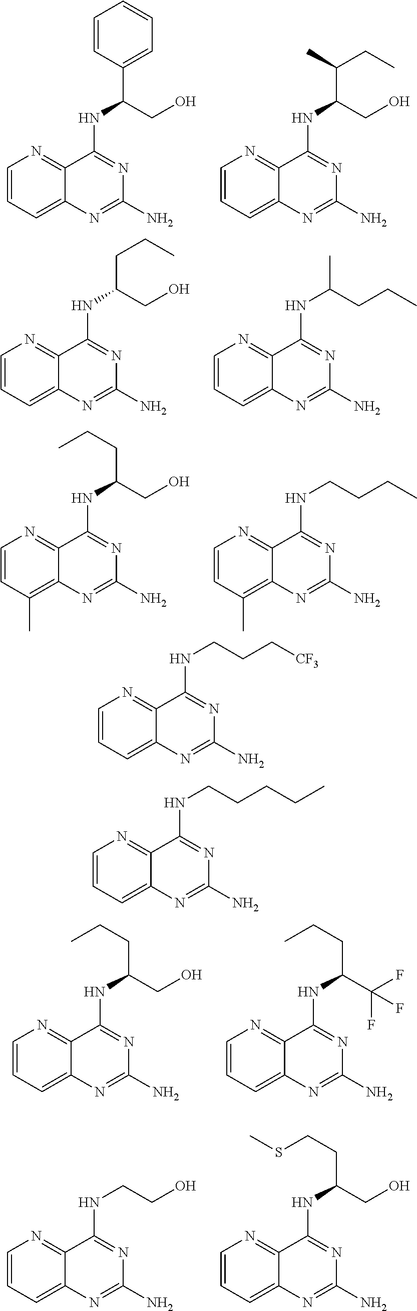

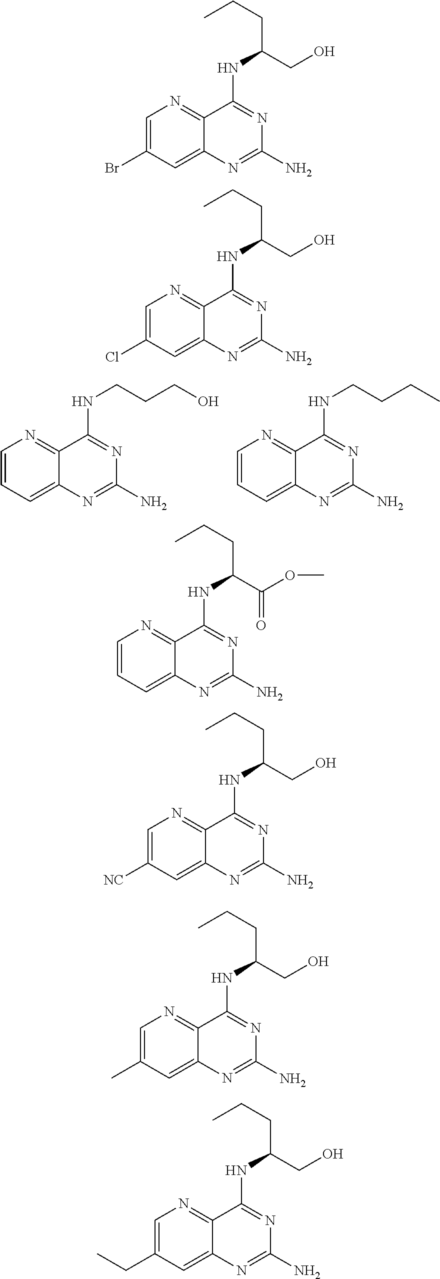

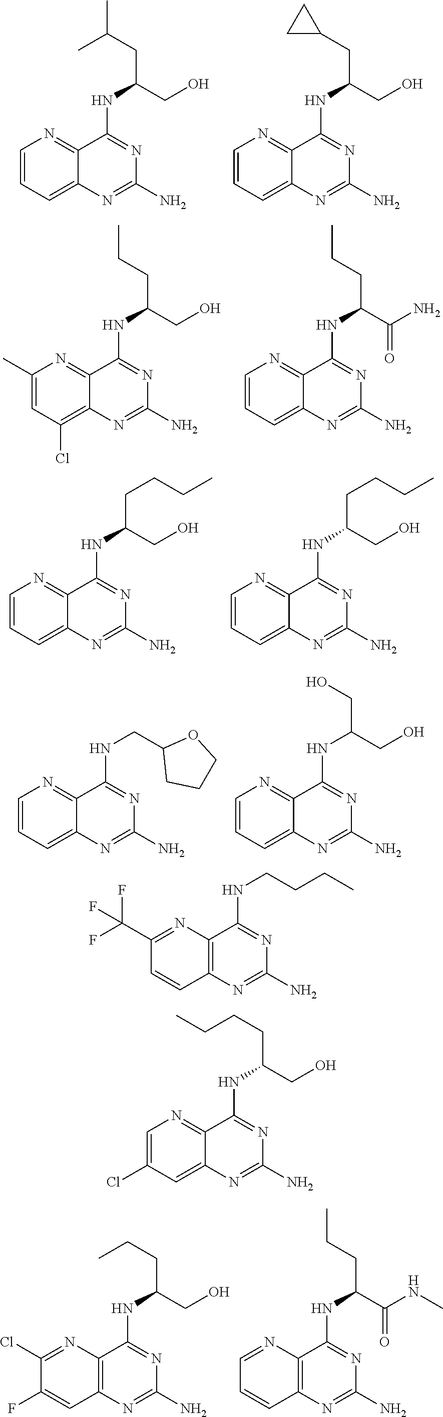

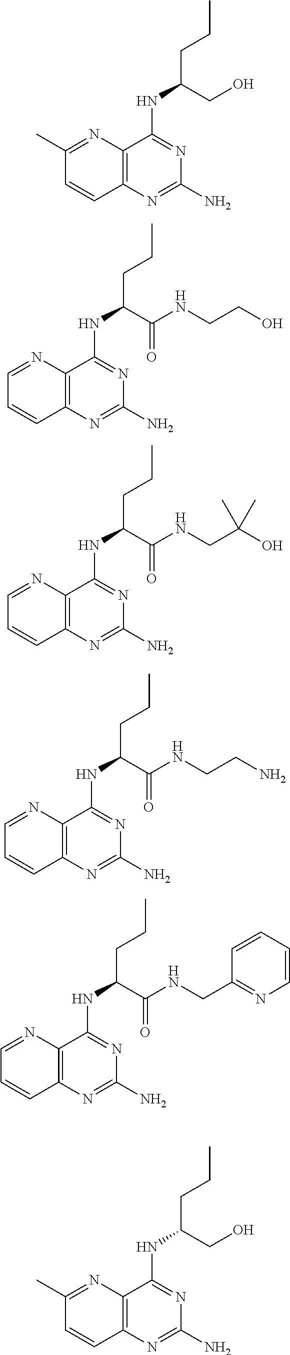

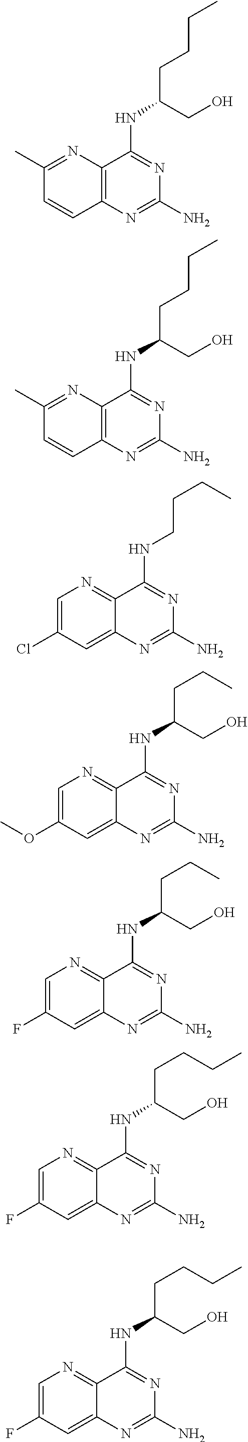

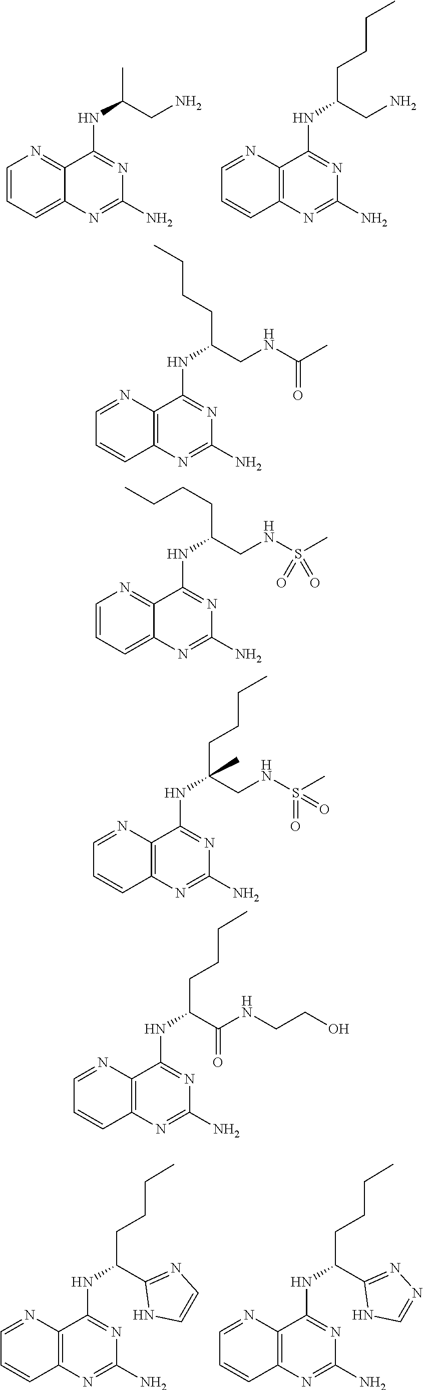

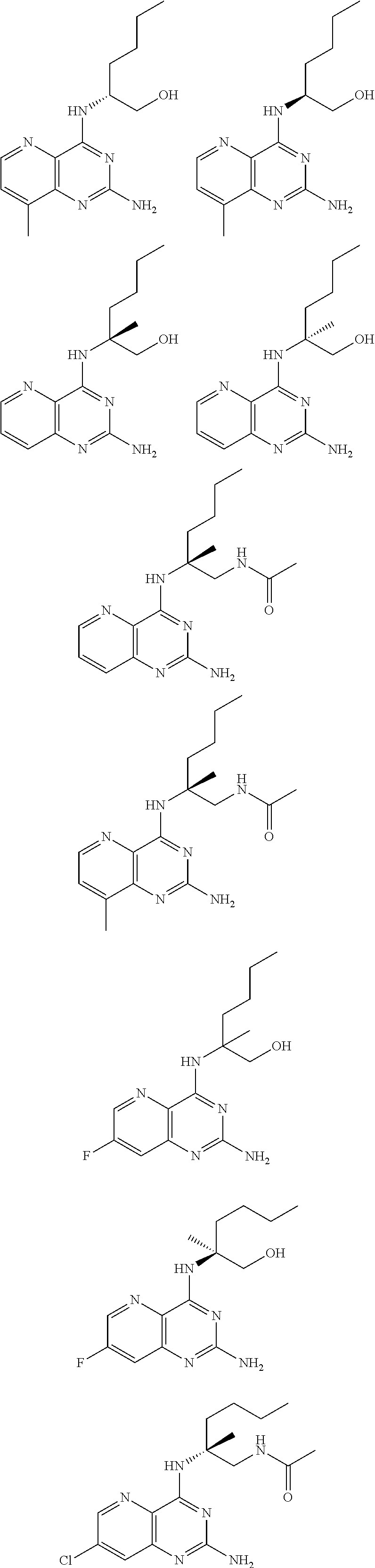

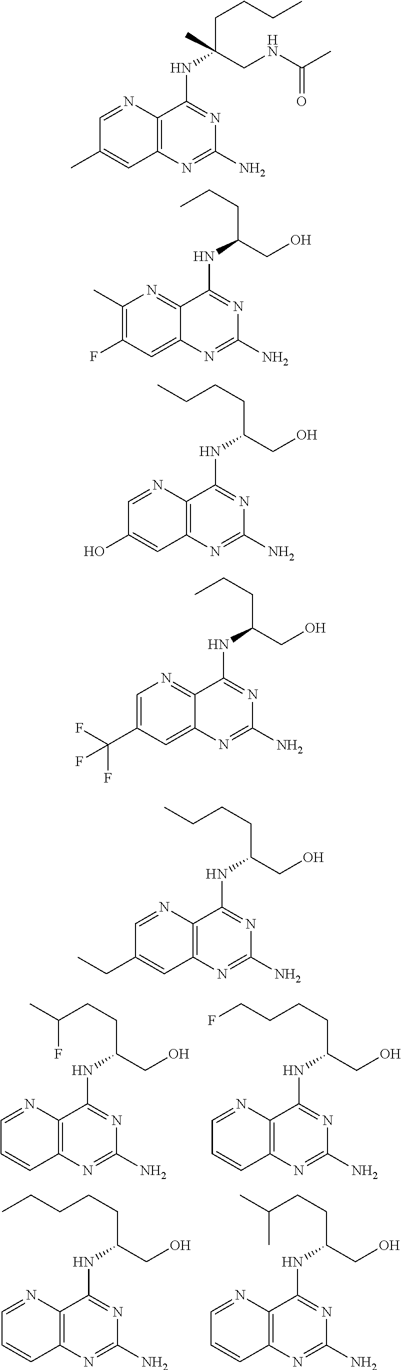

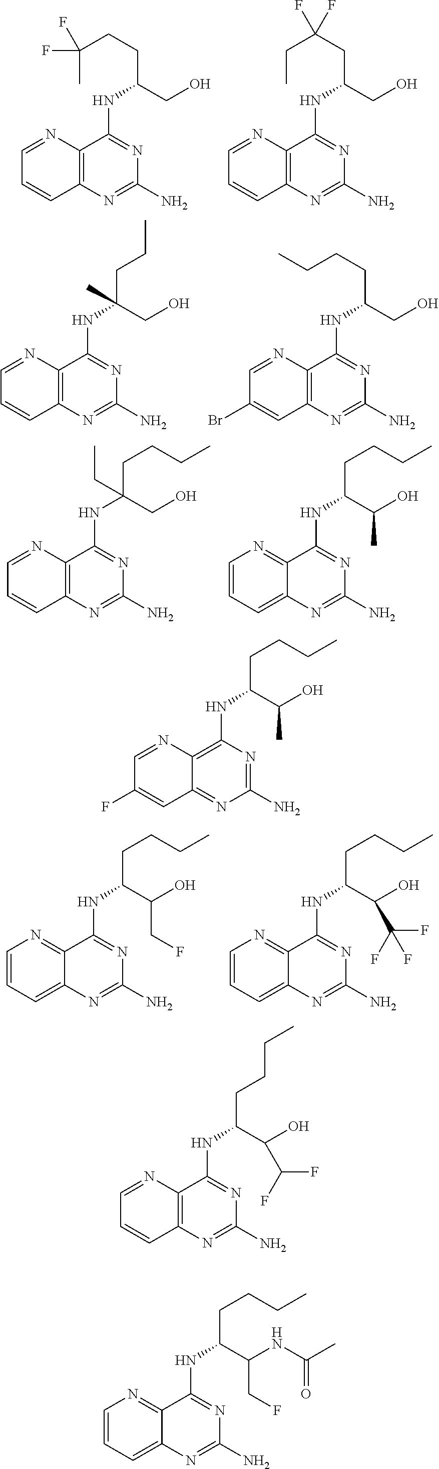

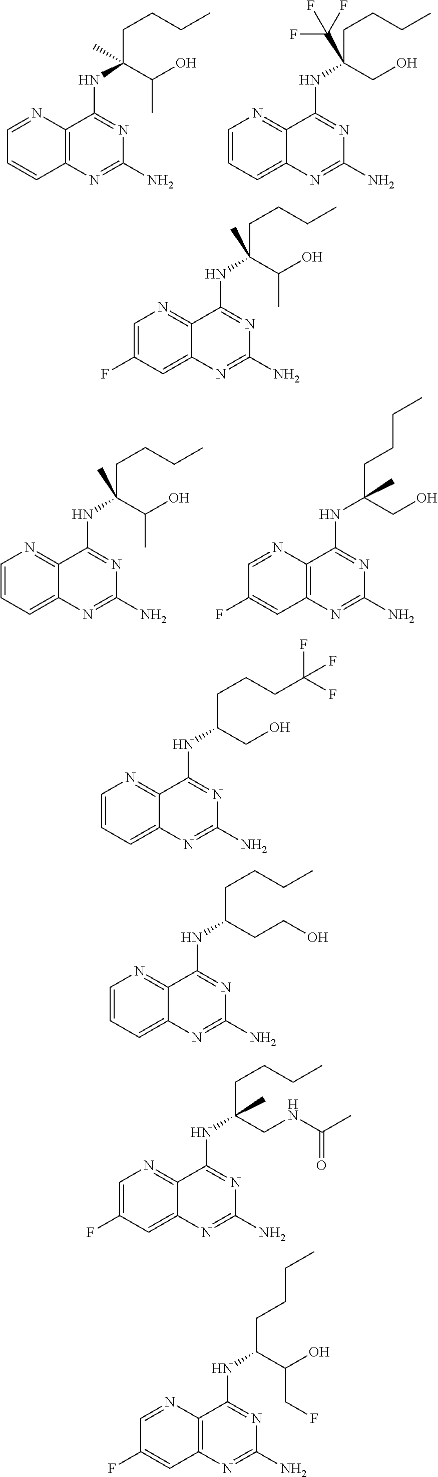

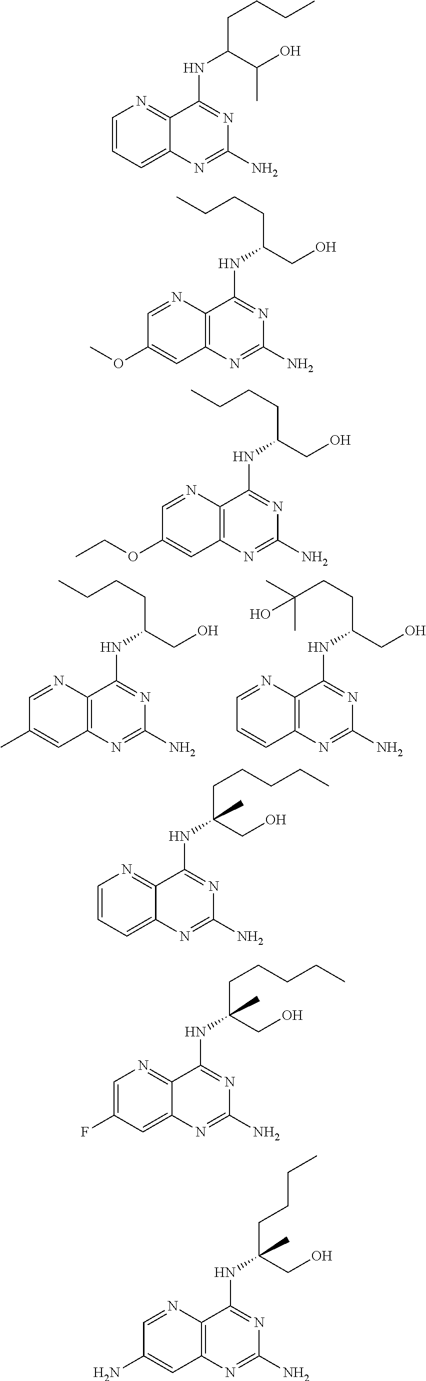

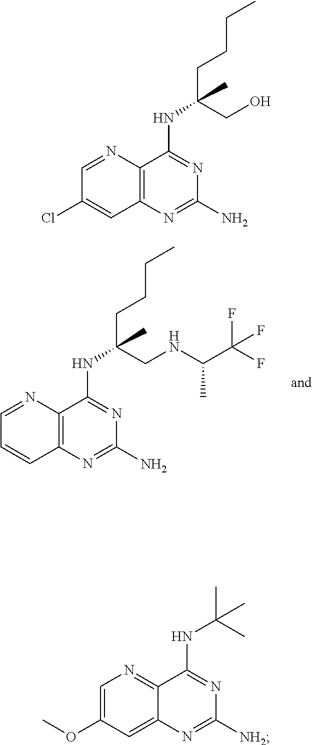

or a pharmaceutically acceptable salt thereof.

Embodiment 15C. The therapeutic combination of any one of claims 1 through 14, wherein the compound is selected from the group consisting of

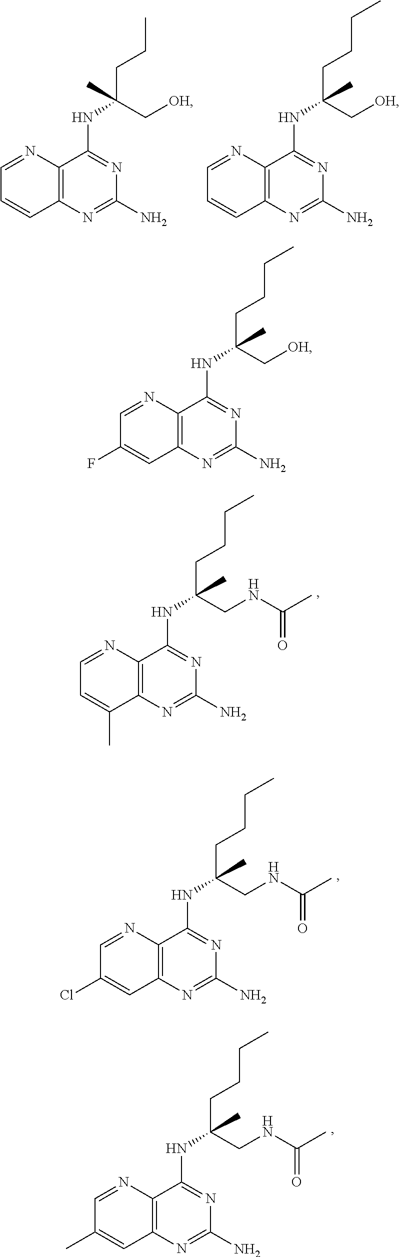

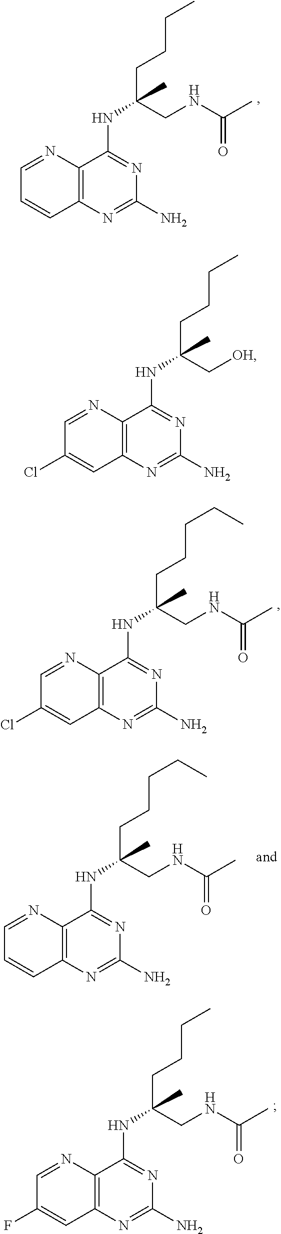

or a pharmaceutically acceptable salt thereof.

Embodiment 16 is a kit comprising the therapeutic combination of any one of embodiments 1 through 15, and instructions for using the therapeutic combination in treating a hepatitis B virus (HBV) infection in a subject in need thereof.

Embodiment 17 is a method of treating a hepatitis B virus (HBV) infection in a subject in need thereof, comprising administering to the subject the therapeutic combination of any one of embodiments 1 through 15.

Embodiment 17a is the method of embodiment 17, wherein the treatment induces an immune response against a hepatitis B virus in a subject in need thereof, preferably the subject has chronic HBV infection.

Embodiment 17b is the method of embodiment 17 or 17a, wherein the subject has chronic HBV infection.

Embodiment 17c is the method of any one of embodiments 17 through 17b, wherein the subject is in need of a treatment of an HBV-induced disease selected from the group consisting of advanced fibrosis, cirrhosis and hepatocellular carcinoma (HCC).

Embodiment 18 is the method of any one of embodiments 17 through 17c, wherein the therapeutic combination is administered by injection through the skin, e.g., intramuscular or intradermal injection, preferably intramuscular injection.

Embodiment 19 is the method of embodiment 18, wherein the therapeutic combination comprises at least one of the first and second non-naturally occurring nucleic acid molecules.

Embodiment 19a is the method of embodiment 19, wherein the therapeutic combination comprises the first and second non-naturally occurring nucleic acid molecules.

Embodiment 20 is the method of embodiment 19 or 19a, wherein the non-naturally occurring nucleic acid molecules are administered to the subject by intramuscular injection in combination with electroporation.

Embodiment 21 is the method of embodiment 19 or 19a, wherein the non-naturally occurring nucleic acid molecules are administered to the subject by a lipid composition, preferably by a lipid nanoparticle.

### Compound K

Unless otherwise indicated, references to substituents (e.g., R¹), compounds, formulas, “Tables”, “Examples”, “Schemes”, and “Aspects” within this section, “Compound K”, are intended to refer to such as defined within this section, “Compound K”.

Dihydropyrimidinyl benzazepine carboxamide compounds having pharmaceutical activity, their manufacture, pharmaceutical compositions containing them and their potential use as medicaments are set forth herein. These compounds may act as TLRS agonists and may therefore be useful as medicaments for the treatment of diseases such as cancer, autoimmune diseases, inflammation, sepsis, allergy, asthma, graft rejection, graft-versus-host disease, immunodeficiencies, and infectious diseases.

In particular, the present invention relates to compounds of the formula

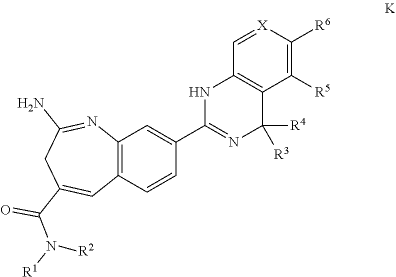

wherein X and R¹ to R⁶ are as described below, or to pharmaceutically acceptable salts thereof.

The compounds are TLR agonists. More particularly, the compounds are TLR8 agonists and may be useful for the treatment and prevention (e.g. vaccination) of cancer, autoimmune diseases, inflammation, sepsis, allergy, asthma, graft rejection, graft-versus-host disease, immunodeficiencies, and infectious diseases.

Toll-like receptors (TLRs) are a family of membrane-spanning receptors that are expressed on cells of the immune system like dendritic cells, macrophages, monocytes, T cells, B cells, NK cells and mast cells but also on a variety of non-immune cells such as endothelial cells, epithelial cells and even tumor cells (Kawai et al., Immunity, 2011, 34, 637-650, Kawai et al., Nat. Immunol., 2010, 11, 373-384). TLRs that recognize bacterial and fungal components are expressed on the cell surface (i.e. TLR1, 2, 4, 5 and 6), while others that recognize viral or microbial nucleic acids like TLR3, 7, 8 and 9 are localized to the endolysosomal/phagosomal compartment (Henessy et al. Nat. Rev. Drug Discovery 2010, 9, 293-307) and predominantly found to be expressed by cells of the myeloid lineage. TLR ligation leads to activation of NF-KB and IRF-dependent pathways with the specific activation sequence and response with respect to the specific TLR and cell type. While TLR7 is mainly expressed in all dendritic cells subtypes (DC and here highly in pDC, plasmacytoid DC) and can be induced in B cells upon IFNcc stimulation (Bekeredjian-Ding et al. J. Immunology 2005, 174:4043-4050), TLR8 expression is rather restricted to monocytes, macrophages and myeloid DC. TLR8 signaling via MyD88 can be activated by bacterial single stranded RNA, small molecule agonists and lately discovered microRNAs (Chen et al. RNA 2013, 19:737-739). The activation of TLR8 results in the production of various pro-inflammatory cytokines such as IL-6, IL-12 and TNF-a as well as enhanced expression of co-stimulatory molecules, such as CD80, CD86, and chemokine receptors (Cros et al. Immunity 2010, 33:375-386). In addition, TLR8 activation can induce type I interferon (ΠTNΓβ) in primary human monocytes (Pang et al. BMC Immunology 2011, 12:55).

Small molecule agonists for both the TLR7 and TLR8 receptor as well as analogs modified for use as vaccine adjuvants or conjugates have been identified in many patents (i.e. WO1992015582, WO2007024612, WO2009111337, WO2010093436, WO2011017611, WO2011068233, WO2011139348, WO2012066336, WO2012167081, WO2013033345, WO2013166110, and US2013202629). Clinical experience has been obtained mainly for TLR7 agonists, but only very few clinical studies focused on using highly specific TLR8 agonists. To date, the only FDA (U.S. Food and Drug Administration)-approved small molecule drug is the TLR7 agonist imiquimod (ALDARA™) as a topical agent for the treatment of genital warts, superficial basal cell carcinoma and actinic keratosis. Systemic application however of the early TLR7 agonists like resiquimod has been abandoned due to intolerable cardiotoxicity observed upon global chemokine stimulation at therapeutic levels (Holldack, Drug Discovery Today, 2013, 1-4). Knowledge about TLR8 agonists is less advanced and mostly restricted to data with early mixed TLR7/8 agonists like resiquimod. For the resiquimod agonist, however, the stimulatory capacity of the TLR7 is superior compared to the activation of the TLR8, so that most of the effects of resiquimod are dominated by the effect of TLR7 activity. More recently, TLR8 specific compounds like VTX-2337 have been described by VentiRX Pharmaceuticals (i.e. WO 2007024612), allowing for the first time to analyse the specific role of TLR8 without activation of TLR7 at the same time. At present there is still a need for small molecule TLR8 agonists, specifically those with improved potency or selectivity.

The present invention is directed to benzazepine compounds with improved cellular potency over known TLR8 agonists of this type for use in the treatment of cancer, preferably solid tumors and lymphomas, and for other uses including the treatment of certain skin conditions or diseases, such as atopic dermatitis, the treatment of infectious diseases, preferably viral diseases, and for use as adjuvants in vaccines formulated for use in cancer therapy or by desensitizing of the receptors by continuous stimulation in the treatment of autoimmune diseases.

The new compounds are characterized by improved cellular potency at TLR8 compared to known TLR8 agonists such as VTX-2337. In addition, these compounds are highly specific towards TLR8 and possess only low or even no activity towards TLR7. Due to the more restricted expression pattern of TLR8 less severe side effects when administered systemically are expected and thus the compounds possess advantageous properties compared to combined TLR7/8 agonists.

The present invention relates to benzazepine-4-carboxamide compounds of the formula

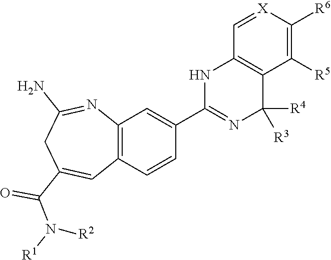

wherein
R¹ is C₃₋₇-alkyl;
R² is C₃₋₇-alkyl or C₃₋₇-cycloalkyl-C₁₋₇-alkyl;
R³ is hydrogen or C₁₋₇-alkyl;
R⁴ is hydrogen or C₁₋₇-alkyl;
R⁵ is selected from the group consisting of hydrogen, halogen, C₁₋₇-alkyl and C₁₋₇-alkoxy;
R⁶ is selected from the group consisting of hydrogen, halogen, C₁₋₇-alkyl and C₁₋₇-alkoxy;
X is N or 7 wherein 7
CR, R is selected from the group consisting of hydrogen, halogen, C₁-7-alkyl and Ci-7-alkoxy; or pharmaceutically acceptable salts thereof.

The invention is also concerned with processes for the manufacture of compounds of formula K. The invention also relates to pharmaceutical compositions comprising a compound of formula K as described above and a pharmaceutically acceptable carrier and/or adjuvant.

A further aspect of the invention is the use of compounds of formula K as therapeutic active substances for the treatment of diseases that can be mediated with TLR agonists, in particular TLR8 agonists. The invention thus also relates to a method for the treatment of a disease that can be mediated with TLR agonists such as for example cancer and autoimmune or infectious diseases.

Unless otherwise defined, all technical and scientific terms used herein have the same meaning as commonly understood by one of ordinary skill in the art to which this invention belongs. Furthermore, the following definitions are set forth to illustrate and define the meaning and scope of the various terms used to describe the invention. The nomenclature used in this application is based on IUPAC systematic nomenclature, unless indicated otherwise.

The term “compound(s) of this invention” and “compound(s) of the present invention” refers to compounds of formula K and solvates or salts thereof (e.g., pharmaceutically acceptable salts).

The term “substituent” denotes an atom or a group of atoms replacing a hydrogen atom on the parent molecule.

The term “lower alkyl” or “C₁₋₇-alkyl”, alone or in combination, signifies a straight-chain or branched-chain optionally substituted alkyl group with 1 to 7 carbon atoms, in particular a straight or branched-chain alkyl group with 1 to 6 carbon atoms and more particularly a straight or branched-chain alkyl group with 1 to 4 carbon atoms. Examples of straight-chain and branched C₁₋₇-alkyl groups are methyl, ethyl, n-propyl, isopropyl, n-butyl, isobutyl, tert-butyl, the isomeric pentyls, the isomeric hexyls and the isomeric heptyls. Methyl, ethyl, n-propyl, isopropyl, n-butyl, isobutyl and tert-butyl are particularly preferred.

The term “C₃₋₇-alkyl” likewise refers to a straight-chain or branched-chain alkyl group with 3 to 7 carbon atoms as defined above, n-propyl is particularly preferred. The term “C₃₋₇-cycloalkyl-C₁₋₇-alkyl” refers to lower alkyl groups as defined above wherein at least one of the hydrogen atoms of the lower alkyl group is replaced by a cycloalkyl group. Among the cycloalkylalkyl groups of particular interest is cyclopropylmethyl.

The term “cycloalkyl” or “C₃₋₇-cycloalkyl” denotes a saturated carbocyclic group containing from 3 to 7 carbon atoms, such as cyclopropyl, cyclobutyl, cyclopentyl, cyclohexyl or cycloheptyl, more particularly cyclopropyl.

The term “C₃₋₇-cycloalkyl-C₁₋₇-alkyl” refers to lower alkyl groups as defined above wherein at least one of the hydrogen atoms of the lower alkyl group is replaced by a cycloalkyl group. Among the lower cycloalkylalkyl groups of particular interest is cyclopropylmethyl.

The term “halogen” refers to fluoro, chloro, bromo and iodo, with fluoro, chloro and bromo being of particular interest. More particularly, halogen refers to fluoro or chloro.

The term “lower alkoxy” or “C₁₋₇-alkoxy” refers to the group R′—O—, wherein R′ is lower alkyl and the term “lower alkyl” has the previously given significance. Examples of lower alkoxy groups are methoxy, ethoxy, n-propoxy, isopropoxy, n-butoxy, isobutoxy, sec.-butoxy and tert-butoxy, in particular methoxy.

The term “pharmaceutically acceptable” denotes an attribute of a material which is useful in preparing a pharmaceutical composition that is generally safe, non-toxic, and neither biologically nor otherwise undesirable and is acceptable for veterinary as well as human pharmaceutical use.

Compounds of formula K can form pharmaceutically acceptable salts. The term “pharmaceutically acceptable salts” refers to those salts which retain the biological effectiveness and properties of the free bases or free acids, which are not biologically or otherwise undesirable. Pharmaceutically acceptable salts include both acid and base addition salts. The salts are for example acid addition salts of compounds of formula K with physiologically compatible mineral acids, such as hydrochloric acid, hydrobromic acid, nitric acid, carbonic acid, sulfuric acid, sulfurous acid or phosphoric acid; or with organic acids, such as methanesulfonic acid, ethanesulfonic acid, p-toluenesulfonic acid, formic acid, acetic acid, propionic acid, glycolic acid, pyruvic acid, oxylic acid, lactic acid, trifluoroacetic acid, citric acid, fumaric acid, maleic acid, malonic acid, tartaric acid, benzoic acid, cinnamic acid, mandelic acid, embonic acid, succinic acid or salicylic acid. In addition, pharmaceutically acceptable salts may be prepared from addition of an inorganic base or an organic base to the free acid. Salts derived from an inorganic base include, but are not limited to, the sodium, potassium, lithium, ammonium, calcium, magnesium, zinc, copper, manganese and aluminium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, histidine, caffeine, procaine, hydrabamine, choline, betaine, ethylendiamine, glucosamine, methylglucamine, theobromine, piperazine, N-ethylpiperidine, piperidine and polyamine resins. The compound of formula K can also be present in the form of zwitterions. Pharmaceutically acceptable salts of compounds of formula K of particular interest are the sodium salts or salts with tertiary amines.

The compounds of formula K can also be solvated, e.g., hydrated. The solvation can be effected in the course of the manufacturing process or can take place e.g. as a consequence of hygroscopic properties of an initially anhydrous compound of formula K (hydration). The term “pharmaceutically acceptable salts” also includes physiologically acceptable solvates.

The term “agonist” denotes a compound that enhances the activity of another compound or receptor site as defined e.g. in Goodman and Gilman's “The Pharmacological Basis of Therapeutics, 7th ed.” in page 35, Macmillan Publ. Company, Canada, 1985. A “full agonist” effects a full response whereas a “partial agonist” effects less than full activation even when occupying the total receptor population. An “inverse agonist” produces an effect opposite to that of an agonist, yet binds to the same receptor binding-site.

The term “half maximal effective concentration” (EC₅₀) denotes the plasma concentration of a particular compound required for obtaining 50% of the maximum of a particular effect in vivo.

The term “therapeutically effective amount” denotes an amount of a compound of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, disease state being treated, the severity or the disease treated, the age and relative health of the subject, the route and form of administration, the judgment of the attending medical or veterinary practitioner, and other factors.

In detail, the present invention relates to compounds of the formula

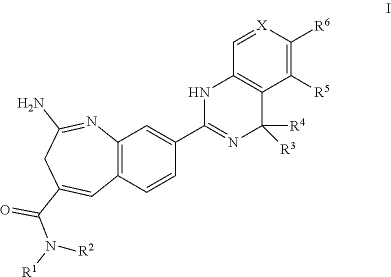

wherein
R¹ is C₃₋₇-alkyl;
R² is C₃₋₇-alkyl or C₃₋₇-cycloalkyl-C₁₋₇-alkyl;
R³ is hydrogen or C₁₋₇-alkyl;
R⁴ is hydrogen or C₁₋₇-alkyl;
R⁵ is selected from the group consisting of hydrogen, halogen, C₁₋₇-alkyl and C₁₋₇-alkoxy;
R⁶ is selected from the group consisting of hydrogen, halogen, C₁₋₇-alkyl and C₁₋₇-alkoxy;
X is N or C—R⁷, wherein R⁷ is selected from the group consisting of hydrogen, halogen, C₁₋₇-alkyl and C₁₋₇-alkoxy;
or pharmaceutically acceptable salts thereof.

In a particular aspect, the invention relates to compounds of formula K, wherein R is n-propyl.

In another aspect, provided are compounds of formula K, wherein R is selected from the group consisting of n-propyl, isobutyl and cyclopropylmethyl. In particular, the invention is concerned with compounds of formula K, wherein R¹ and R² are n-propyl.

In a further aspect, the invention relates to compounds of formula K as defined herein before, wherein R is hydrogen or C₁₋₇-alkyl, in particular hydrogen or methyl. In another aspect, the invention relates to compounds of formula K as defined herein before, wherein R⁴ is hydrogen or C₁₋₇-alkyl, in particular hydrogen or methyl. More particularly, both R³ and R⁴ are hydrogen. In another particular aspect, both R³ and R⁴ are methyl.

In a further aspect, provided are compounds of formula K, wherein X is CR⁷ and R⁷ is selected from the group consisting of hydrogen, halogen, C₁₋₇-alkyl and C₁₋₇-alkoxy. More particularly, R is hydrogen or halogen. In particular, halogen is chloro.

In another aspect, provided are compounds of formula K, wherein X is N.

In a further aspect, the invention relates to compounds of formula K, wherein R⁵ is selected from the group consisting of hydrogen, halogen and C₁₋₇-alkyl. More particularly, R⁵ is hydrogen, chloro, fluoro or methyl.

In another aspect, provided are compounds of formula K, wherein R⁶ is selected from the group consisting of hydrogen, halogen and C₁₋₇-alkoxy. In particular, R⁶ is hydrogen, chloro or methoxy.

Particular compounds of the invention are the following:
2-amino-8-(1,4-dihydroquinazolin-2-yl)-N,N-dipropyl-3H-1-benzazepine-4-carboxamide,
2-amino-8-(1,4-dihydropyrido[3,4-d]pyrimidin-2-yl)-N,N-dipropyl-3H-1-benzazepine-4-carboxamide,
2-amino-N-(cyclopropylmethyl)-8-(1,4-dihydroquinazolin-2-yl)-N-propyl-3H-1-benzazepine-4-carboxamide,
2-amino-8-(1,4-dihydroquinazolin-2-yl)-N-isobutyl-N-propyl-3H-1-benzazepine-4-carboxamide,
2-amino-8-(5-chloro-1,4-dihydroquinazolin-2-yl)-N,N-dipropyl-3H-1-benzazepine-4-carboxamide,
2-amino-8-(7-chloro-1,4-dihydroquinazolin-2-yl)-N,N-dipropyl-3H-1-benzazepine-4-carboxamide,
2-amino-8-(4,4-dimethyl-1H-quinazolin-2-yl)-N,N-dipropyl-3H-1-benzazepine-4-carboxamide,
2-amino-8-(6-chloro-1,4-dihydroquinazolin-2-yl)-N,N-dipropyl-3H-1-benzazepine-4-carboxamide,
2-amino-8-(5-methyl-1,4-dihydroquinazolin-2-yl)-N,N-dipropyl-3H-1-benzazepine-4-carboxamide,
2-amino-8-(5-fluoro-1,4-dihydroquinazolin-2-yl)-N,N-dipropyl-3H-1-benzazepine-4-carboxamide, and
2-amino-8-(6-methoxy-1,4-dihydroquinazolin-2-yl)-N,N-dipropyl-3H-1-benzazepine-4-carboxamide.

A further aspect of the present invention is the process for the manufacture of compounds of formula K as defined above, which process comprises
a) coupling a compound of the formula II

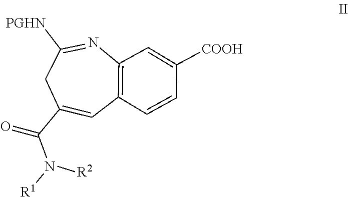

wherein R¹ and R² are as defined in Aspect 1 and PG is a protecting group, with a compound of the formula II

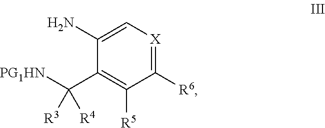

wherein X and R³, R⁴, R⁵ and R⁶ are as defined in Aspect 1 and PG₁ is a protecting group, under basic conditions in the presence of a coupling agent and removing the protecting groups PG and PG₁ under acidic conditions to obtain a compound of the formula K

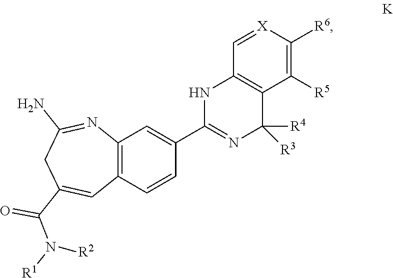

wherein X and R¹ to R⁶ are as defined in Aspect 1, and, if desired, converting the compound obtained into a pharmaceutically acceptable salt.

It will be appreciated, that the compounds of general formula K in this invention may be derivatised at functional groups to provide derivatives which are capable of conversion back to the parent compound in vivo. Physiologically acceptable and metabolically labile derivatives, which are capable of producing the parent compounds of general formula K in vivo are also within the scope of this invention.

In particular, a suitable protecting group PG is an amino-protecting group selected from Boc (tert-butoxycarbonyl), benzyl (Bz) and benzyloxycarbonyl (Cbz). In particular, the protecting group is Boc.

“Removing the protecting group PG under acidic conditions” means treating the protected compound with acids in a suitable solvent, for instance trifluoro acetic acid (TFA) in a solvent such as dichloromethane (DCM) can be employed.

A suitable “coupling agent” for the reaction of compounds of formula II with amines of formula III is selected from the group consisting of N,N′-carbonyldiimidazole (CD I), N,N′-dicyclohexylcarbodiimide (DCC), 1-(3-dimethylaminopropyl)-3-ethylcarbodiimidehydrochloride (EDCI), 1-[bis(dimethylamino)-methylene]-1H-1,2,3-triazolo[4,5-b]pyridinium-3-oxide hexafluorophosphate (HATU), 1-hydroxy-1,2,3-benzotriazole (HOBT), O-benzotriazole-N,N,N′,N′-tetramethyl-uronium-hexafluoro-phosphate (HBTU) or O-benzotriazol-1-yl-N,N,N′,N′-tetramethyluronium tetrafluoroborate (TBTU). In particular, the coupling agent is TBTU. Suitable bases include triethylamine, N-methylmorpholine and, particularly, diisopropylethylamine.

“Under basic conditions” means the presence of a base, in particular a base selected from the group consisting of triethylamine, N-methylmorpholine and, particularly, diisopropylethylamine. Typically, the reaction is carried out in inert solvents such as dimethylformamide or dichloromethane at room temperature.

The invention further relates to compounds of formula K as defined above obtainable according to a process as defined above.

The compounds of the present invention can be prepared by any conventional means. Suitable processes for synthesizing these compounds as well as their starting materials are provided in the schemes below and in the examples. All substituents, in particular, R¹ to R⁴ are as defined above unless otherwise indicated. Furthermore, and unless explicitly otherwise stated, all reactions, reaction conditions, abbreviations and symbols have the meanings well known to a person of ordinary skill in organic chemistry.

A general synthetic route for preparing the compounds of formula K is shown in Scheme 1 below. A symbolizes an aryl ring or a heteroaryl ring.

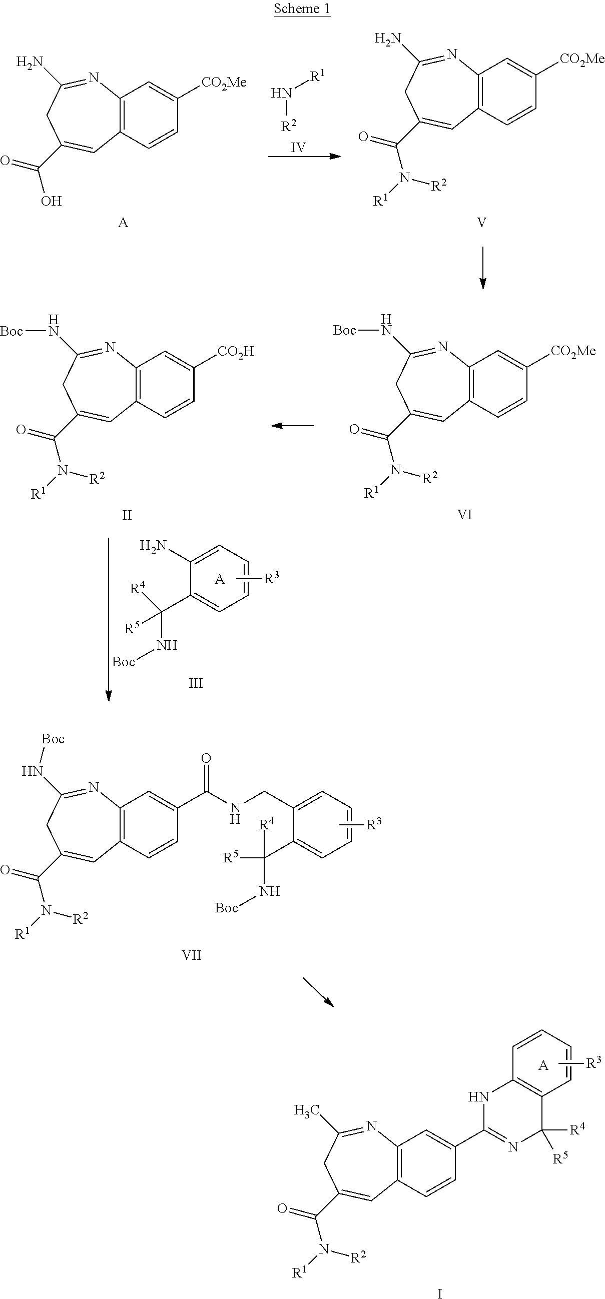

Compounds of formula K can be prepared according to Scheme 1. A coupling reaction between carboxylic acid A and a selected amine IV gives the amide of formula V, which is then protected with an amino protecting group such as Boc to obtain a compound of formula VI. Hydrolysis of the compound of formula VI leads to a carboxylic acid of formula II. The carboxylic acid of formula II is then coupled with a selected aryl or heteroarylamine III to obtain an amide of formula VII. Finally, the compound of formula K is obtained by deprotection of the amino protecting group (e.g. Boc) and in situ cyclization of the amide of formula VII. In some cases, the compound of formula VII may contain an additional acid labile protection group originated from amine IV or amine III, like Boc or TBS, which will be removed also in the final deprotection step.

A coupling reagent, like HBTU, is used to couple the carboxylic acid of formula A and a selected amine IV in the presence of a base, like DIPEA, in a solvent like DCM at ambient or elevated temperature to give a compound of formula V.

Then, the compound of formula V is protected with an amino protecting group, in particular with Boc, to provide a compound of formula VI. The compound of formula VI is hydrolyzed by a base, in particular LiOH, in a suitable solvent, for example a mixed solvent like THF/MeOH/H₂0, at ambient or elevated temperature to obtain a carboxylic acid of formula II.

The carboxylic acid of formula II is then reacted with a selected arylamine or heteroarylamine of formula III under the assistance of a suitable coupling reagent, in particular HATU, in a solvent like DCM and in the presence of a base, in particular DIPEA, at ambient or elevated temperature to result in a compound of formula VII.

Finally, a compound of formula K is obtained by treating the compound of formula VII with TFA in dichloromethane (Boc deprotection and in situ cyclization) and subsequent purification by prep-HPLC.

If one of the starting materials contains one or more functional groups which are not stable or are reactive under the reaction conditions of one or more reaction steps, appropriate protecting groups (PG) (as described e.g. in T. W. Greene et al., Protective Groups in Organic Chemistry, John Wiley and Sons Inc. New York 1999, 3rd edition) can be introduced before the critical step applying methods well known in the art. Such protecting groups can be removed at a later stage of the synthesis using standard methods known in the art. Besides of the Boc protection group at amidine, a compound of formula VII also contains an additional acid labile protection group, like Boc or TBS originated from amine II, which will be also removed in this step.

If one or more compounds of the formula contain chiral centers, compounds of formula K can be obtained as mixtures of diastereomers or enantiomers, which can be separated by methods well known in the art, e.g. (chiral) HPLC or crystallization. Racemic compounds can e.g. be separated into their antipodes via diastereomeric salts by crystallization or by separation of the antipodes by specific chromatographic methods using either a chiral adsorbent or a chiral eluent.

As described herein before, the compounds of formula K of the present invention can be used as medicaments for the treatment of diseases which are mediated by TLR agonists, in particular for the treatment of diseases which are mediated by TLR8 agonists.

The compounds defined in the present invention are agonists of TLR8 receptors in cellular assays in vitro. Accordingly, the compounds of the present invention are expected to be potentially useful agents in the treatment of diseases or medical conditions that may benefit from the activation of the immune system via TLR8 agonists. They are useful in the treatment or prevention of diseases such as cancer, autoimmune diseases, inflammation, sepsis, allergy, asthma, graft rejection, graft-versus-host disease, immunodeficiencies, and infectious diseases.

In more detail, the compounds of formula K of the present invention are useful in oncology, i.e. they may be used in the treatment of common cancers including bladder cancer, head and neck cancer, prostate cancer, colorectal cancer, kidney cancer, breast cancer, lung cancer, ovarian cancer, cervical cancer, liver cancer, pancreatic cancer, bowel and colon cancer, stomach cancer, thyroid cancer, melanoma, skin and brain tumors and malignancies affecting the bone marrow such as leukemias and lymphoproliferative systems, such as Hodgkin's and non-Hodgkin's lymphoma; including the prevention (e.g. vaccination) and treatment of metastatic cancer and tumor recurrences, and paraneoplastic syndromes.

The compounds of formula K of the present invention are also useful in the treatment of autoimmune diseases. An “autoimmune disease” is a disease or disorder arising from and directed against an individual's own tissues or organs or a co-segregate or manifestation thereof or resulting condition therefrom. “Autoimmune disease” can be an organ-specific disease (i.e., the immune response is specifically directed against an organ system such as the endocrine system, the hematopoietic system, the skin, the cardiopulmonary system, the gastrointestinal and liver systems, the renal system, the thyroid, the ears, the neuromuscular system, the central nervous system, etc.) or a systemic disease which can affect multiple organ systems (for example, systemic lupus erythematosus (SLE), rheumatoid arthritis, polymyositis, etc.). In a particular aspect, the autoimmune disease is associated with the skin, muscle tissue, and/or connective tissue.

Particular autoimmune diseases include autoimmune rheumatologic disorders (such as, for example, rheumatoid arthritis, Sjogren's syndrome, scleroderma, lupus such as SLE and lupus nephritis, polymyositis/dermatomyositis, cryoglobulinemia, anti-phospholipid antibody syndrome, and psoriatic arthritis), autoimmune gastrointestinal and liver disorders (such as, for example, inflammatory bowel diseases, ulcerative colitis and Crohn's disease), autoimmune gastritis and pernicious anemia, autoimmune hepatitis, primary biliary cirrhosis, primary sclerosing cholangitis, and celiac disease), vasculitis (such as, for example, ANCA-negative vasculitis and ANCA-associated vasculitis, including Churg-Strauss vasculitis, Wegener's granulomatosis, and microscopic polyangiitis), autoimmune neurological disorders (such as, for example, multiple sclerosis, opsoclonus myoclonus syndrome, myasthenia gravis, neuromyelitis optica, Parkinson's disease, Alzheimer's disease, and autoimmune polyneuropathies), renal disorders (such as, for example, glomerulonephritis, Goodpasture's syndrome, and Berger's disease), autoimmune dermatologic disorders (such as, for example, psoriasis, urticaria, hives, pemphigus vulgaris, bullous pemphigoid, and cutaneous lupus erythematosus), hematologic disorders (such as, for example, thrombocytopenic purpura, thrombotic thrombocytopenic purpura, post-transfusion purpura, and autoimmune hemolytic anemia), atherosclerosis, uveitis, autoimmune hearing diseases (such as, for example, inner ear disease and hearing loss), Behcet's disease, Raynaud's syndrome, organ transplant, and autoimmune endocrine disorders (such as, for example, diabetic-related autoimmune diseases such as insulin-dependent diabetes mellitus (IDDM), Addison's disease, and autoimmune thyroid disease (e.g., Graves' disease and thyroiditis)), allergic conditions and responses, food allergies, drug allergies, insect allergies, rare allergic disorders such as mastocytosis, allergic reaction, eczema including allergic or atopic eczema, asthma such as bronchial asthma and auto-immune asthma, conditions involving infiltration of myeloid cells and T cells and chronic inflammatory responses:

The compounds of formula K of the present invention are also useful in the treatment of infectious diseases. Thus, they may be useful in the treatment of viral diseases, in particular for diseases caused by infection with viruses selected from the group consisting of papilloma viruses, such as human papilloma virus (HPV) and those that cause genital warts, common warts and plantar warts, herpes simplex virus (HSV), molluscum contagiosum, hepatitis B virus (HBV), hepatitis C virus (HCV), Dengue virus, variola virus, human immunodeficiency virus (HIV), cytomegalovirus (CMV), varicella zoster virus (VZV), rhinovirus, enterovirus, adenovirus, coronavirus (e.g. SARS), influenza, mumps and parainfluenza.

They may also be useful in the treatment of bacterial diseases, in particular for diseases caused by infection with bacteria selected from the group consisting of Mycobacterium such as Mycobacterium tuberculosis, Mycobacterium avium and Mycobacterium leprae. The compounds of formula K of the present invention may further be useful in the treatment of other infectious diseases, such as chlamydia, fungal diseases, in particular fungal diseases selected from the group consisting of candidiasis, aspergillosis and cryptococcal meningitis, and parasitic diseases such as Pneumocystis carnii, pneumonia, cryptosporidiosis, histoplasmosis, toxoplasmosis, trypanosome infection and leishmaniasis.

Thus, the expression “diseases which are mediated by TLR8 agonists” means diseases which may be treated by activation of the immune system with TLR8 agonists such as cancer, autoimmune diseases, inflammation, sepsis, allergy, asthma, graft rejection, graft-versus-host disease, immunodeficiencies, and infectious diseases. In particular, the expression “diseases which are mediated by TLR agonists” means cancer, autoimmune diseases, inflammation, sepsis, allergy, asthma, graft rejection, graft-versus-host disease, immunodeficiencies, and infectious diseases.

In a particular aspect, the expression “which are mediated by TLR8 agonists” relates to cancer selected from the group consisting of bladder cancer, head and neck cancer, liver cancer, prostate cancer, colorectal cancer, kidney cancer, breast cancer, lung cancer, ovarian cancer, cervical cancer, pancreatic cancer, bowel and colon cancer, stomach cancer, thyroid cancer, melanoma, skin and brain tumors and malignancies affecting the bone marrow such as leukemias and lymphoproliferative systems, such as Hodgkin's and non-Hodgkin's lymphoma; including the prevention (e.g. vaccination) and treatment of metastatic cancer and tumor recurrences, and paraneoplastic syndromes.

The invention also relates to pharmaceutical compositions comprising a compound of formula K as defined above and a pharmaceutically acceptable carrier and/or adjuvant. More specifically, the invention relates to pharmaceutical compositions useful for the treatment of diseases which are which are mediated by TLR8 agonists.

Further, the invention relates to compounds of formula K as defined above for use as therapeutically active substances, particularly as therapeutically active substances for the treatment of diseases which are which are mediated by TLR8 agonists. In particular, the invention relates to compounds of formula K for use in the treatment of cancers or autoimmune diseases or infectious diseases selected from the group consisting of viral diseases, bacterial diseases, fungal diseases and parasitic diseases.

In another aspect, the invention relates to a method for the treatment a of diseases which are mediated by TLR8 agonists, which method comprises administering a therapeutically active amount of a compound of formula K to a human being or animal. In particular, the invention relates to a method for the treatment of cancers and infectious diseases selected from the group consisting of viral diseases, bacterial diseases, fungal diseases and parasitic diseases.

The invention further relates to the use of compounds of formula K as defined above for the treatment of diseases which are mediated by TLR8 agonists.

In addition, the invention relates to the use of compounds of formula K as defined above for the preparation of medicaments for the treatment of diseases which are mediated by TLR8 agonists. In particular, the invention relates to the use of compounds of formula K as defined above for the preparation of medicaments for the treatment of cancers or autoimmune diseases or infectious diseases selected from the group consisting of viral diseases, bacterial diseases, fungal diseases and parasitic diseases.

In a further aspect, compounds of formula K can be in combination with one or more additional treatment modalities in a regimen for the treatment of cancer.

Combination therapy encompasses, in addition to the administration of a compound of the invention, the adjunctive use of one or more modalities that are effective in the treatment of cancer. Such modalities include, but are not limited to, chemotherapeutic agents, immunotherapeutics, anti-angiogenic agents, cytokines, hormones, antibodies, polynucleotides, radiation and photodynamic therapeutic agents. In a specific aspect, combination therapy can be used to prevent the recurrence of cancer, inhibit metastasis, or inhibit the growth and/or spread of cancer or metastasis. As used herein, “in combination with” means that the compound of formula K is administered as part of a treatment regimen that comprises one or more additional treatment modalities as mentioned above. The invention thus also relates to a method for the treatment of cancer, which method comprises administering a therapeutically active amount of a compound of formula K in combination with one or more other pharmaceutically active compounds to a human being or animal.

Compounds of formula K can be used alone or in combination with one or more additional treatment modalities in treating autoimmune diseases.

Combination therapy encompasses, in addition to the administration of a compound of the invention, the adjunctive use of one or more modalities that aid in the prevention or treatment of autoimmune diseases. Such modalities include, but are not limited to, chemotherapeutic agents, immunotherapeutics, anti-angiogenic agents, cytokines, hormones, antibodies, polynucleotides, radiation and photodynamic therapeutic agents. As used herein, “in combination with” means that the compound of formula K is administered as part of a treatment regimen that comprises one or more additional treatment modalities as mentioned above. The invention thus also relates to a method for the treatment of autoimmune diseases, which method comprises administering a therapeutically active amount of a compound of formula K in combination with one or more other pharmaceutically active compounds to a human being or animal.

In a further aspect, compounds of formula K can be used alone or in combination with more additional treatment modalities in treating infectious diseases.

Combination therapy encompasses, in addition to the administration of a compound of the invention, the adjunctive use of one or more modalities that aid in the prevention or treatment of infectious diseases. Such modalities include, but are not limited to, antiviral agents, antibiotics, and anti-fungal agents. As used herein, “in combination with” means that the compound of formula K is administered as part of a treatment regimen that comprises one or more additional treatment modalities as mentioned above. The invention thus also relates to a method for the treatment of infectious diseases, which method comprises administering a therapeutically active amount of a compound of formula K in combination with one or more other pharmaceutically active compounds to a human being or animal.

### Pharmacological Test

The following tests were carried out in order to determine the activity of the compounds of formula K:

For TLR8 and TLR7 activity testing, HEK-Blue human TLR8 or TLR7 cells (Invivogen, San Diego, Calif., USA) are used, respectively. These cells are designed for studying the stimulation of human TLR8 or TLR7 by monitoring the activation of NF-κB. A SEAP (secreted embryonic alkaline phosphatase) reporter gene is placed under the control of the IFN-b minimal promoter fused to five NF-κB and AP-1-binding sites. Therefore the reporter expression is regulated by the NF-κB promoter upon stimulation of human TLR8 or TLR7 for 20 hours. The cell culture supernatant SEAP reporter activity was determined using Quanti Blue kit (Invivogen, San Diego, Ca, USA) at a wavelength of 640 nm, a detection medium that turns purple/blue in the presence of alkaline phosphatase. EC₅₀ values were determined using Activity Base analysis (ID Business Solution, Limited).

VTX-133 and VTX-135 are two examples described in International Patent Application No. WO 2011/022509 and their activity in HEK-blue human TLR7 and TLR8 cells are shown in Table 1.

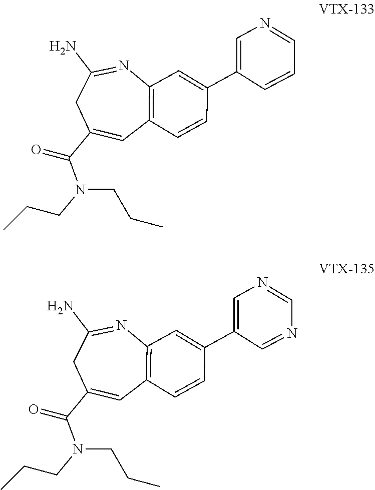 [SMILES: CCCN(CCC)C(=O)C1=Cc2ccc(-c3cccnc3)cc2N=C(N)C1 ; CCCN(CCC)C(=O)C1=Cc2ccc(-c3cncnc3)cc2N=C(N)C1]

Of note, the new compounds described in this patent have improved cellular potency at TLR8 compared to known TLR8 agonists such as VTX-133 and VTX-135 described in WO 2011022509. In addition these compounds are highly specific towards TLR8 with no appreciable activity towards TLR7. Thus, they are expected to possess advantageous properties compared to combined TLR7/8 agonists due to the more restricted expression pattern of TLR8 resulting in less served side effects when administered systemically.

The compounds according to formula K have an activity (EC₅₀ value) in the above assay for human TLR8 in the range of 0.001 μM to 0.03 μM, more particularly of 0.001 μM to 0.015 μM, whereas the activity (EC₅₀ value) in the above assay for human TLR7 is greater than 100 μM, meaning the compounds show very high selectivity towards human TLR8.

For example, the following compounds showed the following EC₅₀ values in the assay described above:

TABLE 1

| | human TLR8 | human TLR7 |
|---|---|---|
| Example | EC₅₀ [μM] | EC₅₀ [μM] |
| VTX-133 | 0.077 | 1.86 |
| VTX-135 | 0.039 | 3.61 |
| 1 | 0.003 | >100 |
| 2 | 0.003 | >100 |
| 3 | 0.006 | >100 |
| 4 | 0.011 | >100 |
| 5 | 0.011 | >100 |
| 6 | 0.009 | >100 |
| 7 | 0.007 | >100 |
| 8 | 0.006 | >100 |
| 9 | 0.001 | >100 |
| 10 | 0.003 | >100 |
| 11 | 0.002 | >100 |

### Pharmaceutical Compositions

The compounds of formula K and their pharmaceutically acceptable salts can be used as medicaments, e.g., in the form of pharmaceutical preparations for enteral, parenteral or topical administration. The compounds of formula K and their pharmaceutically acceptable salts may be administered by systemic (e.g., parenteral) or local (e.g., topical or intralesional injection) administration, in some instances, the pharmaceutical formulation is topically, parenterally, orally, vaginally, intrauterine, intranasal, or by inhalation administered. As described herein, certain tissues may be preferred targets for the TLR8 agonist. Thus, administration of the TLR8 agonist to lymph nodes, spleen, bone marrow, blood, as well as tissue exposed to virus, are preferred sites of administration.

In one aspect, the pharmaceutical formulation comprising the compounds of formula K or its pharmaceutically acceptable salts is administered parenterally. Parenteral routes of administration include, but are not limited to. transdermal, transmucosal. nasopharyngeal, pulmonary and direct injection. Parenteral administration by injection may be by any parenteral injection route, including, but not limited to. intravenous (IV), including bolus and infusion (e.g., fast or slow), intraperitoneal (IP), intramuscular (IM), subcutaneous (SC) and intradermal (ID) routes. Transdermal and transmucosal administration may be accomplished by, for example, inclusion of a carrier (e.g., dimethyisu!foxide, DMSO), by application of electrical impulses (e.g., iontophoresis) or a combination thereof. A variety of devices are available for transdermal administration which may be used. Formulations of the compounds of formula K suitable for parenteral administration are generally formulated in USP water or water for injection and may further comprise pH buffers, salts bulking agents, preservatives, and other pharmaceutically acceptable excipients.

Transdermal administration is accomplished by application of a cream, rinse, gel, etc. capable of allowing the TLR8 agonist to penetrate the skin and enter the blood stream. Compositions suitable for transdermal administration include, but are not limited to, pharmaceutically acceptable suspensions, oils, creams and ointments applied directly to the skin or incorporated into a protective carrier such as a transdermal device (so-called “patch”). Examples of suitable creams, ointments etc. can be found, for instance, in the Physician's Desk Reference. Transdermal transmission may also be accomplished by iontophoresis, for example using commercially available patches which deliver their product continuously through unbroken skin for periods of several days or more. Use of this method allows for controlled transmission of pharmaceutical compositions in relatively great concentrations, permits infusion of combination drugs and allows for con tern poraneous use of an absorption promoter. Administration via the transdermal and transmucosal routes may be continuous or pulsatile.

Pulmonary administration is accomplished by inhalation, and includes delivery routes such as intranasal, transbronchial and transalveolar routes. Formulations of compounds of formula K suitable for administration by inhalation including, but not limited to, liquid suspensions for forming aerosols as well as powder forms for dry powder inhalation delivery systems are provided. Devices suitable for administration by inhalation include, but are not limited to, atomizers, vaporizers, nebulizers, and dry powder inhalation delivery devices. Other methods of delivering to respiratory mucosa include delivery of liquid formulations, such as by nose drops. Administration by inhalation is preferably accomplished in discrete doses (e.g., via a metered dose inhaler), although delivery similar to an infusion may be accomplished through use of a nebulizer.

The compounds of formula K and pharmaceutically acceptable salts thereof may also be administered orally, e.g., in the form of tablets, coated tablets, dragees, hard and soft gelatine capsules.

The production of the pharmaceutical preparations can be effected in a manner which will be familiar to any person skilled in the art by bringing the described compounds of formula K and their pharmaceutically acceptable salts, optionally in combination with other therapeutically valuable substances, into a galenical administration form together with suitable, non-toxic, inert, therapeutically compatible solid or liquid carrier materials and, if desired, usual pharmaceutical adjuvants.

Suitable carrier materials are not only inorganic carrier materials, but also organic carrier materials. Thus, for example, lactose, corn starch or derivatives thereof, talc, stearic acid or its salts can be used as carrier materials for tablets, coated tablets, dragees and hard gelatine capsules. Suitable carrier materials for soft gelatine capsules are, for example, vegetable oils, waxes, fats and semi-solid and liquid polyols (depending on the nature of the active ingredient no carriers might, however, be required in the case of soft gelatine capsules). Suitable carrier materials for the production of solutions and syrups are, for example, water, polyols, sucrose, invert sugar and the like. Suitable carrier materials for injection solutions are, for example, water, alcohols, polyols, glycerol and vegetable oils. Suitable carrier materials for suppositories are, for example, natural or hardened oils, waxes, fats and semi-liquid or liquid polyols. Suitable carrier materials for topical preparations are glycerides, semi-synthetic and synthetic glycerides, hydrogenated oils, liquid waxes, liquid paraffins, liquid fatty alcohols, sterols, polyethylene glycols and cellulose derivatives.

Usual stabilizers, preservatives, wetting and emulsifying agents, consistency-improving agents, flavour-improving agents, salts for varying the osmotic pressure, buffer substances, solubilizers, colorants and masking agents and antioxidants come into consideration as pharmaceutical adjuvants.

The dosage of the compounds of formula K can vary within wide limits depending on the disease to be controlled, the age and the individual condition of the patient and the mode of administration, and will, of course, be fitted to the individual requirements in each particular case. For adult patients a daily dosage of about 1 to 1000 mg, especially about 1 to 300 mg, comes into consideration. Depending on severity of the disease and the precise pharmacokinetic profile the compound could be administered with one or several daily dosage units, e.g., in 1 to 3 dosage units.

The pharmaceutical preparations conveniently contain about 1-500 mg, preferably 1-100 mg, of a compound of formula K.

The following examples C1 to C3 illustrate typical compositions of the present invention, but serve merely as representative thereof.

### Example C1

Film coated tablets containing the following ingredients can be manufactured in a conventional manner:

| | |
|---|---|
| Ingredients | Per tablet |

Kernel:

| | | |
|---|---|---|
| Compound of formula K | 10.0 mg | 200.0 mg |
| Microcrystalline | cellulose 23.5 mg | 43.5 mg |
| Lactose hydrous | 60.0 mg | 70.0 mg |
| Povidone K30 | 12.5 mg | 15.0 mg |
| Sodium starch glycolate | 12.5 mg | 17.0 mg |
| Magnesium stearate | 1.5 mg | 4.5 mg |
| (Kernel Weight) | 120.0 mg | 350.0 mg |

Film Coat:

| | | |
|---|---|---|
| Hydroxypropyl methyl | 3.5 mg | 7.0 mg |
| cellulose | | |
| Polyethylene glycol 6000 | 0.8 mg | 1.6 mg |
| Talc | 1.3 mg | 2.6 mg |
| Iron oxide (yellow) | 0.8 mg | 1.6 mg |
| Titanium dioxide | 0.8 mg | 1.6 mg |

The active ingredient is sieved and mixed with microcrystalline cellulose and the mixture is granulated with a solution of polyvinylpyrrolidone in water. The granulate is mixed with sodium starch glycolate and magnesiumstearate and compressed to yield kernels of 120 or 350 mg respectively. The kernels are lacquered with an aqueous solution/suspension of the above mentioned film coat.

### Example C2

Capsules containing the following ingredients can be manufactured in a conventional manner:

| Ingredients | Per capsule |
|---|---|
| Compound of formula K | 25.0 mg |
| Lactose | 150.0 mg |
| Maize starch | 20.0 mg |
| Talc | 5.0 mg |

The components are sieved and mixed and filled into capsules of size 2.

### Example C3

Injection solutions can have the following composition:

| Compound of formula K | 3.0 mg |
|---|---|
| Polyethylene glycol 400 | 150.0 mg |
| Acetic acid | q.s. ad pH 5.0 |
| Water for injection solutions | ad 1.0 ml |

The active ingredient is dissolved in a mixture of Polyethylene Glycol 400 and water for injection (part). The pH is adjusted to 5.0 by acetic acid. The volume is adjusted to 1.0 ml by addition of the residual amount of water. The solution is filtered, filled into vials using an appropriate overage and sterilized.

The following examples serve to illustrate the present invention in more detail. They are, however, not intended to limit its scope in any manner.

### EXAMPLES

Abbreviations used therein:
Boc₂0=di-ie/t-butyl dicarbonate, Boc=i-butyl carbamate, calc'd=calculated, CD₃OD=deuterated methanol, d=day, DIPEA=N,N-diisopropylethylamine, DCM=dichloromethane, DMAP: 4-dimethylaminopyridine, DMF-DMA: N,N-dimethylformamide dimethyl acetal, EA=ethyl acetate or EtOAc, EC₅₀=half maximal effective concentration, h or hr=hour, HBTU=O-(benzotriazol-1-yl)-N,N,N′,N′-tetramethyluronium hexafluorophosphate, DMAP=4-dimethylaminopyridine, HATU=(1-[Bis(dimethylamino)methylene]-1H-1,2,3-triazolo[4,5-b]pyridinium 3-oxid hexafluorophosphate), HPLC-UV=high performance liquid chromatography with ultraviolet detector, Hz=hertz, mg=milligram, MHz=megahertz, min=minute(s), mL=milliliter, mm=millimeter, mM=mmol/L, mmol=millimole, MS=mass spectrometry, MW=molecular weight, NMR=nuclear magnetic resonance, PE=petroleum ether, prep-HPLC=preparative high performance liquid chromatography, rt=room temperature, sat.=sat., TBS=ie/t-butyldimethylsilyl, sxt=sextet, TEA=triethylamine, TFA=trifluoroacetic acid, THF=tetrahydrofuran, μM=micromole/L, μιη=micrometer, UV=ultraviolet detector, OD=optical density, TLR8=toll-like receptor 8, TLR7=toll-like receptor 7, NF-κB=nuclear factor kappa-light-chain-enhancer of activated B cells, SEAP=secreted embryonic alkaline phosphatase, IFN-β=interferon-beta.

### Example A—Preparation of Key Intermediate A

### 2-Amino-8-methoxycarbonyl-3H-1-benzazepine-4-carboxylic Acid

A detailed synthetic route is provided in Scheme 2.

### a) Preparation of Compound B

To a solution of methyl 4-methyl-3-nitrobenzoate (100 g, 0.51 mol) in DMF (1 L) was added DMF-DMA (73 g, 0.61 mol). The reaction mixture was heated to 105° C. for 18 hrs. Then the solvent was removed in vacuo to give methyl 4-(2-(dimethylamino)vinyl)-3-nitrobenzoate (compound B, 127 g, crude) which was used in the next step without purification. MS: calc'd 251 (M+H)⁺, measured 251 (M+H)⁺.

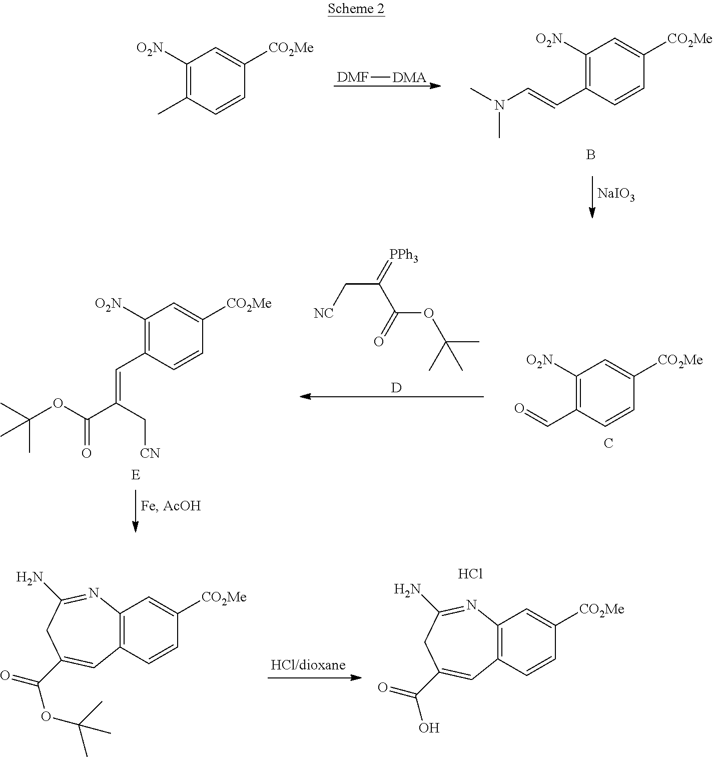

### b) Preparation of Compound C

To a solution of NaIO₄ (327 g, 1.53 mol) in a mixed solvent of THF (1.3 L) and water (2.0 L) was added a THF (0.7 L) solution of methyl 4-(2-(dimethylamino)vinyl)-3-nitrobenzoate (compound A, 127 g, 0.51 mol) at 10° C. After the reaction mixture was stirred at 25° C. for 18 hrs, the mixture was filtered and then extracted with EA. The organic layer was washed with brine, dried over anhydrous Na₂S0₄, filtered and concentrated to give the crude product. The crude product was purified by silica gel column chromatography (PE:EA=20:1-10:1) to give methyl 4-formyl-3-nitrobenzoate (compound C, 84 g, 79%) as a yellow solid. MS: calc'd 210 (M+H)⁺, measured 210 (M+H)⁺.

### c) Preparation of Compound D

To a solution of tert-butyl 2-(triphenylphosphoranylidene)acetate (300 g, 0.797 mol) in EA (2 L) was added 2-bromoacetonitrile (57 g, 0.479 mol) at 25° C. The reaction was heated to reflux for 18 hrs. After it was cooled to ambient temperature, the solid was filtered and the filtrate was concentrated. The residue was purified by triturating from EA and PE (200 mL, 2.5:1) to give the desired product ie/t-butyl 3-cyano-2-(triphenylphosphoranylidene)propanoate (compound D, 125 g, 63%) as a white solid. MS: calc'd 416 (M+H)⁺, measured 416 (M+H)⁺.

### d) Preparation of Compound E

To a solution of 4-formyl-3-nitrobenzoate (compound C, 50 g, 0.24 mol) in toluene (600 mL) was added ie/t-butyl 3-cyano-2-(triphenylphosphoranylidene)propanoate (compound D, 109 g, 0.26 mol) at 25° C. After the reaction mixture was stirred at 25° C. for 18 hrs, it was cooled in ice-bath for 1 hr. The precipitate was collected and dried to give the desired product as a white solid. The filtrate was concentrated and treated with EtOH (120 mL). The undissolved material was filtered and the filtrate was concentrated to give an additional batch of the desired product. These two batches were combined to give methyl 4-(3-(tert-butoxy)-2-(cyanomethyl)-3-oxoprop-1-en-1-yl)-3-nitrobenzoate (compound E, 60 g, 72%). MS: calc'd 347 (M+H)⁺, measured 347 (M+H)⁺.

### e) Preparation of Compound F

To a solution of methyl 4-(3-(tert-butoxy)-2-(cyanomethyl)-3-oxoprop-1-en-1-yl)-3-nitrobenzoate (compound E, 30 g, 87 mmol) in AcOH (450 mL) was added Fe powder (29.1 g, 520 mmol) at 60° C. After the reaction mixture was heated at 85° C. for 3 hrs, it was filtered through celite and the precipitate was washed with acetic acid. The filtrate was concentrated in vacuo and the residue was carefully basified with aqueous sat. NaHCO₃ solution (3M) mL). Then EA (600 mL) was added. The mixture was filtered through celite and the precipitate was washed with EA (200 mL). The filtrate was then washed with water, dried over Na₂S0₄ and concentrated in vacuo to get 4-ie/t-butyl 8-methyl 2-amino-3H-benzo[b]azepine-4,8-dicarboxylate (compound F, 25 g, 93%) as a light yellow solid. MS: calc'd 317 (M+H)⁺, measured 317 (M+H)⁺.

### f) Preparation of Compound A

To a solution of 4-ie/t-butyl 8-methyl 2-amino-3H-benzo[b]azepine-4,8-dicarboxylate (compound F, 25 g, 80 mmol) in dioxane (400 mL) was added a 1 M solution of HQ in dioxane (600 mL) at 0° C. After the reaction mixture was stirred at 25° C. for 18 hrs, it was concentrated in vacuo to give 2-amino-8-(methoxycarbonyl)-3H-benzo[b]azepine-4-carboxylic acid hydrochloride (compound A, 25 g, crude) which was used in the next step without any purification. MS: calc'd 261 (M+H)⁺, measured 261 (M+H)⁺.

### Example B—Preparation of Key Intermediate J

### 2-(tert-butoxycarbonylamino)-4-(dipropylcarbamoyl)-3H-1-benzazepine-8-carboxylic Acid

A detailed synthetic route is provided in Scheme 3.

### g) Preparation of Compound G

To a mixture of 2-amino-8-(methoxycarbonyl)-3H-benzo[b]azepine-4-carboxylic acid hydrochloride (compound A, 19 g, 64 mmol), HBTU (29 g, 77 mmol), DIPEA (33 g, 257 mmol) in DMF (400 mL) was added di-w-propylamine (13 g, 128 mmol) at 0° C. After the reaction mixture was stirred at 20° C. for 2 hrs. it was quenched with sat. NH₄Cl (500 mL), diluted with H₂0 (1 L), and extracted with EA (300 mL×3). The combined organic layers were washed with brine (300 mL×2), dried over Na₂S0₄ and concentrated to give the crude product. The crude product was purified by silica gel column chromatography (PE:EA=1:1) to give methyl 2-amino-4-(dipropylcarbamoyl)-3H-benzo[b]azepine-8-carboxylate (compound G, 18 g, 82%) as a yellow solid. MS: calc'd 344 (M+H)⁺, measured 344 (M+H)⁺.

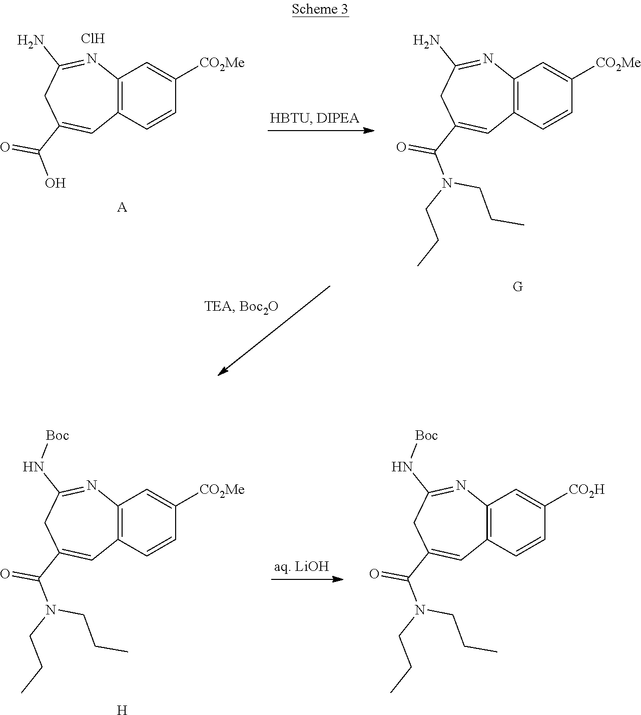

### h) Preparation of Compound H

To a mixture of methyl 2-amino-4-(dipropylcarbamoyl)-3H-benzo[b]azepine-8-carboxylate (compound G, 18 g, 53 mmol) and TEA (16 g, 157 mmol) in DCM (300 mL) was added Boc₂0 (17 g, 79 mmol) at 0° C. After the mixture was stirred at 20° C. for 16 hrs, it was quenched with sat. NH₄Cl (300 mL), diluted with H₂0 (500 mL), and extracted with DCM (100 mL×3). The combined organic layers were washed with brine (100 mL×2), dried over Na₂S0₄ and concentrated to give the crude product. The crude product was purified by silica gel column chromatography (PE:EA=3:1) to give methyl 2-((tert-butoxycarbonyl)amino)-4-(dipropylcarbamoyl)-3H-benzo[b]azepine-8-carboxylate (compound H, 21 g, yield: 91%) as a yellow solid. MS: calc'd 444 (M+H)⁺, measured 444 (M+H)⁺.

### i) Preparation of Compound J

To a solution of methyl 2-((tert-butoxycarbonyl)amino)-4-(dipropylcarbamoyl)-3H-benzo[b]azepine-8-carboxylate (compound H, 5.0 g, 11.3 mmol) in THF/H₂0 (1/1, 100 mL) was added aq. LiOH solution (1 M, 17 mL, 17 mmol) at 0° C. Then the mixture was warmed to 25° C. and stirred for 6 hrs. The mixture was poured into ice-water (150 mL), acidified with aq. citric acid (5%) to pH=5 and extracted with EtOAc (100 mL×3). The combined organic layers were washed with brine (100 mL×2), dried over Na₂S0₄ and concentrated in vacuo to give 2-(tert-butoxycarbonylamino)-4-(dipropylcarbamoyl)-3H-1-benzazepine-8-carboxylic acid (compound J, 4.0 g, 83.3%) as a yellow solid. 1H NMR (400 MHz, DMSO-d₆) δ ppm=7.78-7.72 (m, 1H), 7.64 (dd, /=1.5, 8.0 Hz, 1H), 7.55 (d, /=8.3 Hz, 1H), 6.93-6.89 (m, 1H), 3.14 (s, 6H), 1.54 (br. s., 4H), 1.44 (s, 9H), 0.80 (br. s., 6H). MS: calc'd 430 (M+H)⁺, measured 430 (M+H)⁺.

### Example 1

### 2-Amino-8-(1,4-dihydroquinazolin-2-yl)-N,N-dipropyl-3H-1-benzazepine-4-carboxamide

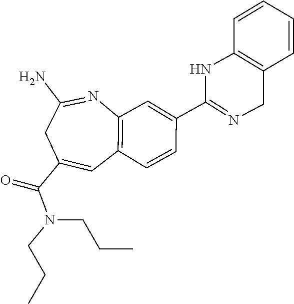 [SMILES: CCCN(CCC)C(=O)C1=Cc2ccc(C3=NCc4ccccc4N3)cc2N=C(N)C1]

Example 1 can be prepared according to general procedure in scheme 1. A detailed synthetic route is provided in Scheme 4.

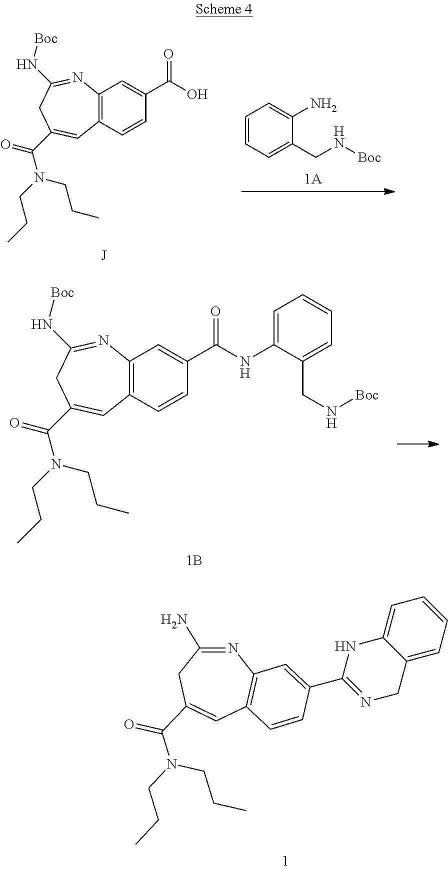 [SMILES: CCCN(CCC)C(=O)C1=Cc2ccc(C(=O)O)cc2N=C(NC(=O)OC(C)(C)C)C1 ; CC(C)(C)OC(=O)NCc1ccccc1N ; CCCN(CCC)C(=O)C1=Cc2ccc(C(=O)Nc3ccccc3CNC(=O)OC(C)(C)C)cc2N=C(NC(=O)OC(C)(C)C)C1 ; CCCN(CCC)C(=O)C1=Cc2ccc(C3=NCc4ccccc4N3)cc2N=C(N)C1]

Preparation of compound IB:

To a solution of 2-(tert-butoxycarbonylamino)-4-(dipropylcarbamoyl)-3H-1-benzazepine-8-carboxylic acid (compound J, 200 mg, 0.465 mmol) in DMF (4.0 mL) was added HATU (177 mg, 0.550 mmol), DIPEA (84 mg, 0.60 mmol) and tert-butyl N-[(2-aminophenyl)methyl]-carbamate (compound 1A, 122 mg, 0.55 mmol). The solution was stirred at 50° C. for 24 hrs. Water (10 mL) was added and the mixture was extracted with EA (10 mL×2). The organic layer was washed by brine (10 mL×2), dried over Na₂S0₄ and concentrated in vacuo to give the crude product. The residue was purified by prep-TLC to give tert-butyl N-[[2-[[2-(tert-butoxycarbonyl-amino)-4-(dipropylcarbamoyl)-3H-1-benzazepine-8-carbonyl]amino]phenyl]methyl]carbamate (compound IB, 15 mg) as a yellow solid. MS: calc'd 634 (M+H)⁺, measured 634 (M+H)⁺.

### Preparation of Example 1

To a solution of tert-butyl N-[[2-[[2-(tert-butoxycarbonylamino)-4-(dipropylcarbamoyl)-3H-1-benzazepine-8-carbonyl]amino]phenyl]methyl]carbamate (compound IB, 15 mg, 0.023 mmol) in DCM (1.0 ml) was added TFA (0.3 mL). The reaction was stirred at 20° C. for 2 hrs. Then the reaction mixture was concentrated and the residue was purified by preparative HPLC to give 2-amino-8-(1,4-dihydroquinazolin-2-yl)-N,N-dipropyl-3H-1-benzazepine-4-carboxamide (Example 1, 12 mg) as a yellow solid. 1H NMR (400 MHz, MeOD) δ ppm=7.89-7.85 (m, 3H), 7.42-7.36 (m, 2H), 7.29-7.25 (m, 2H), 7.16 (s, 1H), 5.01 (s, 2H), 3.48 (m, 4H), 3.41 (s, 2H), 1.74-1.69 (m, 4H), 1.00-0.93 (m, 6H). MS: calc'd 416 (M+H)⁺, measured 416 (M+H)⁺.

### Example 2

### 2-Amino-8-(1,4-dihydropyrido[3,4-d]pyrimidin-2-yl)-N,N-dipropyl-3H-1-benzazepine-4-carboxamide

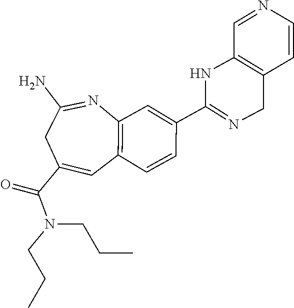 [SMILES: CCCN(CCC)C(=O)C1=Cc2ccc(C3=NCc4ccncc4N3)cc2N=C(N)C1]

The title compound was prepared in analogy to Example 1 by using tert-butyl ((3-aminopyridin-4-yl)methyl)carbamate instead of tert-butyl 2-aminobenzylcarbamate. Example 2 was obtained (16 mg) as a yellow solid. 1H NMR (400 MHz, MeOD) δ ppm=8.44 (m, 2H), 7.84-7.80 (m, 3H), 7.33-7.27 (m, 1H), 7.01 (s, 1H), 4.94 (s, 2H), 3.41-3.16 (m, 6H), 1.75-1.55 (m, 4H), 1.15-0.8 (m, 6H). MS: calc'd 417 (M+H)⁺, measured 417 (M+H)⁺.

### Example 3

### 2-Amino-N-(cyclopropylmethyl)-8-(1,4-dihydroquinazolin-2-yl)-N-propyl-3H-1-benzazepine-4-carboxamide

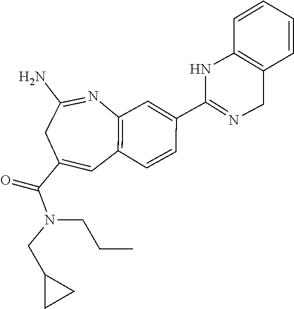 [SMILES: CCCN(CC1CC1)C(=O)C1=Cc2ccc(C3=NCc4ccccc4N3)cc2N=C(N)C1]

A detailed synthetic route is provided in Scheme 5.

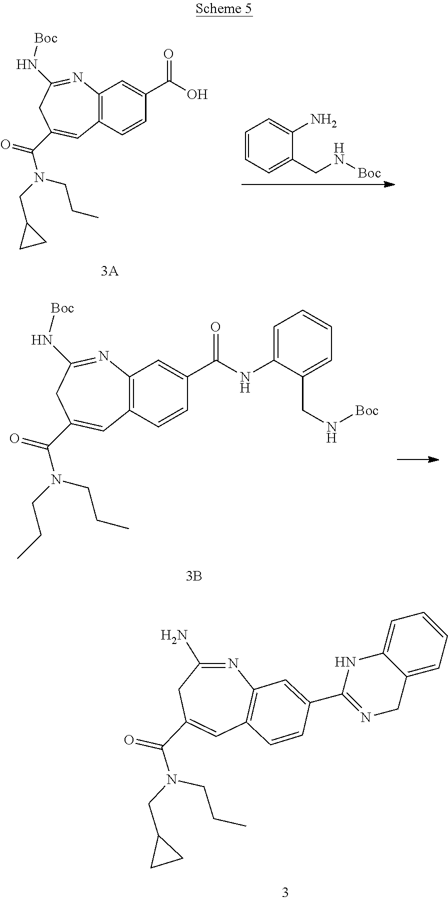 [SMILES: CCCN(CC1CC1)C(=O)C1=Cc2ccc(C(=O)O)cc2N=C(NC(=O)OC(C)(C)C)C1 ; CC(C)(C)OC(=O)NCc1ccccc1N ; CCCN(CCC)C(=O)C1=Cc2ccc(C(=O)Nc3ccccc3CNC(=O)OC(C)(C)C)cc2N=C(NC(=O)OC(C)(C)C)C1 ; CCCN(CC1CC1)C(=O)C1=Cc2ccc(C3=NCc4ccccc4N3)cc2N=C(N)C1]

The title compound was prepared in analogy to Example 1 by using 2-((tert-butoxycarbonyl)amino)-4-((cyclopropylmethyl)(propyl)carbamoyl)-3H-benzo[b]azepine-8-carboxylic acid (compound 3A) instead of 2-(tert-butoxycarbonylamino)-4-(dipropylcarbamoyl)-3H-1-benzazepine-8-carboxylic acid (compound J). Example 3 was obtained (2 mg) as a white solid. 1H NMR (400 MHz, MeOD) δ ppm=7.87-7.85 (m, 3H), 7.42-7.36 (m, 2H), 7.30-7.24 (m, 2H), 7.17 (s, IH), 5.04 (s, 2H), 3.61-3.59 (m, 2H), 3.44-3.41 (m, 4H), 1.76-1.74 (m, 2H), 1.31 (br s, IH), 1.11-0.97 (br s, 3H), 0.64 (br s, 2H), 0.31 (br s, 2H). MS: calc'd 428 (M+H)⁺, measured 428 (M+H)⁺.

### Preparation of Compound 3A:

The title compound was prepared in analogy to key intermediate J of Example B by using N-(cyclopropylmethyl)propan-1-amine instead of di-w-propylamine.

### Example 4

### 2-Amino-8-(1,4-dihydroquinazolin-2-yl)-N-isobutyl-N-propyl-3H-1-benzazepine-4-carboxamide

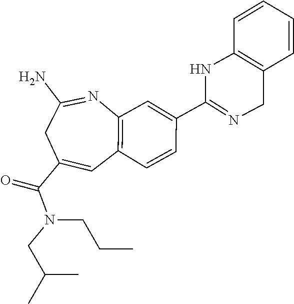 [SMILES: CCCN(CC(C)C)C(=O)C1=Cc2ccc(C3=NCc4ccccc4N3)cc2N=C(N)C1]

The title compound was prepared in analogy to Example 3 by using 2-methyl-N-propylpropan-1-amine instead of N-(cyclopropylmethyl)propan-1-amine. Example 4 was obtained (4.5 mg) as a yellow solid. 1H NMR (400 MHz, MeOD) δ ppm=7.87-7.83 (m, 3H), 7.35-7.27 (m, 4H), 7.14 (s, 1H), 5.03 (s, 2H), 3.38 (br s, 6H), 1.75-1.6 (m, 3H), 0.92 (br s, 9H). MS: calc'd 430 (M+H)⁺, measured 430 (M+H)⁺.

### Example 5

### 2-Amino-8-(5-chloro-1,4-dihydroquinazolin-2-yl)-N,N-dipropyl-3H-1-benzazepine-4-carboxamide

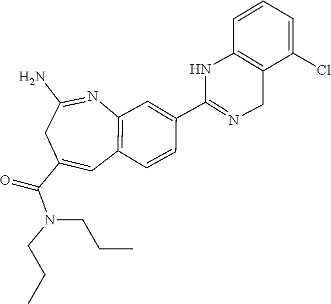 [SMILES: CCCN(CCC)C(=O)C1=Cc2ccc(C3=NCc4c(Cl)cccc4N3)cc2N=C(N)C1]

The title compound was prepared in analogy to Example 1 by using tert-butyl 2-amino-6-chlorobenzylcarbamate (compound 5C) instead of ie/t-butyl N-[(2-aminophenyl)methyl]-carbamate. Example 5 was obtained (19 mg) as a white solid. 1H NMR (400 MHz, MeOD) δ ppm=7.76-7.72 (m, 3H), 7.79-7.78 (m, 2H), 7.03 (s, 2H), 4.92 (s, 2H), 3.37 (br s, 6H), 1.61-1.59 (m, 4H), 1.00-0.93 (m, 6H). MS: calc'd 450 (M+H)⁺, measured 450 (M+H)⁺.

The preparation of compound 5C is shown in scheme 6.

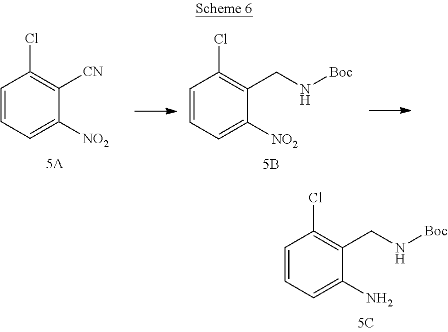 [SMILES: N#Cc1c(Cl)cccc1[N+](=O)[O-] ; CC(C)(C)OC(=O)NCc1c(Cl)cccc1[N+](=O)[O-] ; CC(C)(C)OC(=O)NCc1c(N)cccc1Cl]

To a solution of 2-chloro-6-nitrobenzonitrile (compound 5A, 2.0 g, 10.98 mmol) in THF (20 mL) was added BH₃.THF (33 mL, 32.9 mmol). The solution was refluxed for 3 hrs. The reaction solution was cooled under ice-bath and then MeOH (20 mL) was added dropwise. The solution was stirred for 30 min and then Boc₂0 (2.63 g, 12.1 mmol) was added. The solution was stirred at 20° C. for 3 hrs. After the reaction solution was concentrated in vacuo, the residue was purified by column chromatography (PE/EtOAc=20/1-5/1) to give crude tert-butyl 2-chloro-6-nitrobenzylcarbamate (compound 5B, 1.4 g, 44.5%) as yellow oil, which was used for the next step directly. MS: calc'd 287 (M+H)⁺, measured 287 (M+H)⁺.

To a solution of ie/t-butyl 2-chloro-6-nitrobenzylcarbamate (compound 5B, 1.4 g, 4.9 mmol) in MeOH (70 mL) was added NH₄C1 (3.6 g, 68.5 mmol) and Zn (2.79 g, 44.0 mmol). The solution was stirred at 20° C. for 2 hrs. The reaction solution was concentrated in vacuo. Water (30 mL) was added, and the mixture was extracted with EA (30 mL). The organic layer was washed with brine, dried over anhydrous Na₂SO₄ and concentrated in vacuo to give tert-butyl 2-amino-6-chlorobenzylcarbamate (compound 5C, 800 mg, 64%) as a yellow solid, which was used for the next step directly. MS: calc'd 257 (M+H)⁺, measured 257 (M+H)⁺.

### Example 6

### 2-Amino-8-(7-chloro-1,4-dihydroquinazolin-2-yl)-N,N-dipropyl-3H-1-benzazepine-4-carboxamide

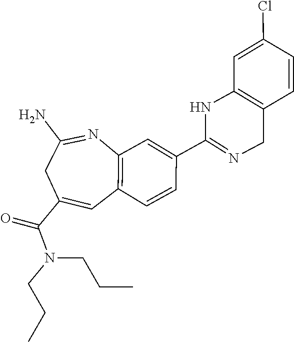 [SMILES: CCCN(CCC)C(=O)C1=Cc2ccc(C3=NCc4ccc(Cl)cc4N3)cc2N=C(N)C1]

The title compound was prepared in analogy to Example 5 by using tert-butyl 2-amino-4-chlorobenzylcarbamate instead of tert-butyl N-[(2-aminophenyl)methyl]carbamate. Example 5 was obtained (5 mg) as a white solid. 1H NMR (400 MHz, MeOD) δ ppm=7.88-7.84 (m, 3H), 7.35-7.28 (m, 3H), 7.15 (s, 1H), 5.01 (s, 2H), 3.48-3.40 (m, 6H), 1.75-1.68 (m, 4H), 0.96 (br s, 6H). MS: calc'd 450 (M+H)⁺, measured 450 (M+H)⁺.

### Example 7

### 2-Amino-8-(4,4-dimethyl-1H-quinazolin-2-yl)-N,N-dipropyl-3H-1-benzazepine-4-carboxamide

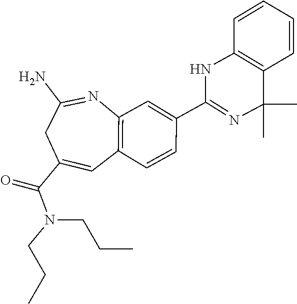 [SMILES: CCCN(CCC)C(=O)C1=Cc2ccc(C3=NC(C)(C)c4ccccc4N3)cc2N=C(N)C1]

The title compound was prepared in analogy to Example 1 by using 2-(2-aminopropan-2-yl)aniline instead of tert-butyl N-[(2-aminophenyl)methyl]carbamate. Example 7 was obtained (18 mg) as a white solid. 1H NMR (400 MHz, MeOD) δ ppm=7.86 (brs, 3H), 7.51-7.25 (m, 4H), 7.17 (s, 1H), 3.55-3.40 (m, 6H), 1.86 (s, 6H), 1.73-1.71 (m, 4H), 0.97 (br s, 6H). MS: calc'd 444 (M+H)⁺, measured 444 (M+H)⁺.

### Example 8

### 2-Amino-8-(6-chloro-1,4-dihydroquinazolin-2-yl)-N,N-dipropyl-3H-1-benzazepine-4-carboxamide

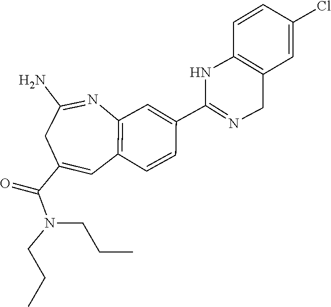 [SMILES: CCCN(CCC)C(=O)C1=Cc2ccc(C3=NCc4cc(Cl)ccc4N3)cc2N=C(N)C1]

The title compound was prepared in analogy to Example 5 by using tert-butyl 2-amino-5-chlorobenzylcarbamate instead of tert-butyl 2-amino-6-chlorobenzylcarbamate. Example 8 was obtained (6 mg) as a white solid. 1H NMR (400 MHz, MeOD) δ ppm=7.86-7.83 (m, 3H), 7.42-7.23 (m, 3H), 7.15 (s, 1H), 5.02 (s, 2H), 3.49-3.39 (m, 6H), 1.74-1.69 (m, 4H), 1.00-0.92 (br s, 6H). MS: calc'd 450 (M+H)⁺, measured 450 (M+H)⁺.

### Example 9

### 2-Amino-8-(5-methyl-1,4-dihydroquinazolin-2-yl)-N,N-dipropyl-3H-1-benzazepine-4-carboxamide

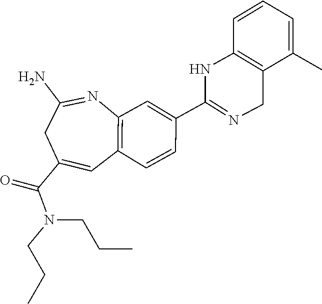 [SMILES: CCCN(CCC)C(=O)C1=Cc2ccc(C3=NCc4c(C)cccc4N3)cc2N=C(N)C1]

The title compound was prepared in analogy to Example 5 by using tert-butyl 2-amino-6-methylbenzylcarbamate instead of tert-butyl 2-amino-6-chlorobenzylcarbamate. Example 9 was obtained (29 mg) as a white solid. 1H NMR (400 MHz, MeOD) δ ppm=7.87-7.85 (m, 3H), 7.30-7.28 (m, 1H), 7.20-7.16 (m, 2H), 7.06-7.04 (s, 1H), 4.99 (s, 2H), 3.48 (br s, 4H), 3.41 (s, 2H), 2.30 (s, 3H), 1.75-1.69 (m, 4H), 0.99-0.93 (br s, 6H). MS: calc'd 430 (M+H)⁺, measured 430 (M+H)⁺.

### Example 10

### 2-Amino-8-(5-fluoro-1,4-dihydroquinazolin-2-yl)-N,N-dipropyl-3H-1-benzazepine-4-carboxamide

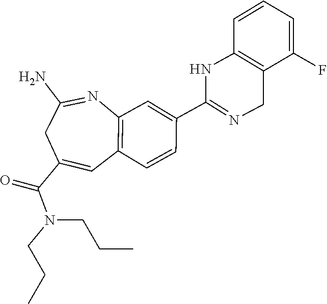 [SMILES: CCCN(CCC)C(=O)C1=Cc2ccc(C3=NCc4c(F)cccc4N3)cc2N=C(N)C1]

The title compound was prepared in analogy to Example 5 by using tert-butyl 2-amino-6-fluorobenzylcarbamate instead of ie/t-butyl 2-amino-6-chlorobenzylcarbamate. Example 10 was obtained (5 mg) as a white solid. 1H NMR (400 MHz, MeOD) δ ppm=7.87-7.83 (m, 3H), 7.46-7.40 (m, 1H), 7.13-7.06 (m, 3H), 5.03 (s, 2H), 3.46-3.31 (br s, 4H), 3.30 (s, 2H), 1.72-1.67 (m, 4H), 0.98-0.97 (br s, 6H). MS: calc'd 434 (M+H)⁺, measured 434 (M+H)⁺.

### Example 11

### 2-Amino-8-(6-methoxy-1,4-dihydroquinazolin-2-yl)-N,N-dipropyl-3H-1-benzazepine-4-carboxamide

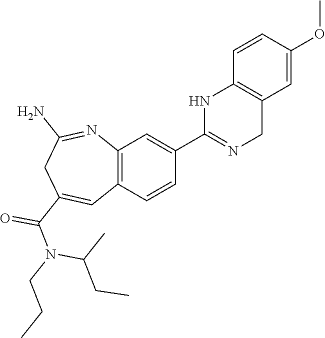 [SMILES: CCCN(C(=O)C1=Cc2ccc(C3=NCc4cc(OC)ccc4N3)cc2N=C(N)C1)C(C)CC]

The title compound was prepared in analogy to Example 5 by using tert-butyl 2-amino-5-methoxybenzylcarbamate instead of tert-butyl 2-amino-6-chlorobenzylcarbamate. Example 11 was obtained (37 mg) as a white solid. 1H NMR (400 MHz, MeOD) δ ppm=7.85-7.84 (m, 3H), 7.21-7.15 (m, 2H), 6.99-6.96 (m, 1H), 6.87-6.86 (m, IH), 5.00 (s, 2H), 3.85 (s, 3H), 3.48 (br s, 4H), 3.41 (s, 2H), 2.30 (s, 3H), 1.74-1.69 (m, 4H), 1.00-0.93 (br s, 6H). MS: calc'd 446 (M+H)⁺, measured 446 (M+H)⁺.

### Aspects:

Aspect 1. A benzazepine carboxamide compound of the formula

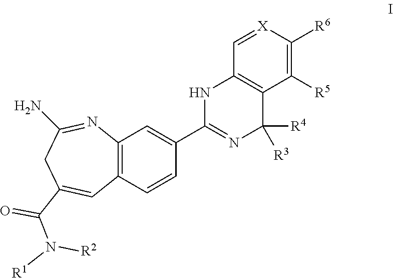

wherein
R¹ is C₃₋₇-alkyl;
R is C₃₋₇-alkyl or C₃₋₇-cycloalkyl-C₁₋₇-alkyl;
R is hydrogen or C₁₋₇-alkyl;
R⁴ is hydrogen or C₁₋₇-alkyl;
R⁵ is selected from the group consisting of hydrogen, halogen, C₁₋₇-alkyl and C₁₋₇-alkoxy;
R⁶ is selected from the group consisting of hydrogen, halogen, C₁₋₇-alkyl and C₁₋₇-alkoxy;
X is N or CR⁷, wherein R⁷ is selected from the group consisting of hydrogen, halogen, C₁₋₇-alkyl and C₁₋₇-alkoxy;
or pharmaceutically acceptable salts thereof.
Aspect 2. The compound of Aspect 1, wherein R¹ is n-propyl.
Aspect 3. The compound of Aspects 1 or 2, wherein R is selected from n-propyl, isobutyl and cyclopropylmethyl.
Aspect 4. The compound of any one of Aspects 1 to 3, wherein R¹ and R² are n-propyl.
Aspect 5. The compound of any one of Aspects 1 to 4, wherein R³ and R⁴ are hydrogen.
Aspect 6. The compound of any one of Aspects 1 to 4, wherein R³ and R⁴ are methyl.
Aspect 7. The compound of any one of Aspects 1 to 6, wherein X is CR and R is selected from the group consisting of hydrogen, halogen, C₁₋₇-alkyl and C₁₋₇-alkoxy.
Aspect 8. The compound of Aspect 7, wherein R is hydrogen or halogen.
Aspect 9. The compound of any one of Aspects 1 to 6, wherein X is N.
Aspect 10. The compound of any one of Aspects 1 to 9, wherein R⁵ is selected from the group consisting of hydrogen, halogen and C₁₋₇-alkyl.
Aspect 11. The compound of any one of Aspects 1 to 10, wherein R⁶ is selected from the group consisting of hydrogen, halogen and C₁₋₇-alkoxy.
Aspect 12. A compound of the formula K according to Aspect 1, selected from the group of
2-amino-8-(1,4-dihydroquinazolin-2-yl)-N,N-dipropyl-3H-1-benzazepine-4-carboxamide,
2-amino-8-(1,4-dihydropyrido[3,4-d]pyrimidin-2-yl)-N,N-dipropyl-3H-1-benzazepine-4-carboxamide,
2-amino-N-(cyclopropylmethyl)-8-(1,4-dihydroquinazolin-2-yl)-N-propyl-3H-1-benzazepine-4-carboxamide,
2-amino-8-(1,4-dihydroquinazolin-2-yl)-N-isobutyl-N-propyl-3H-1-benzazepine-4-carboxamide,
2-amino-8-(5-chloro-1,4-dihydroquinazolin-2-yl)-N,N-dipropyl-3H-1-benzazepine-4-carboxamide,
2-amino-8-(7-chloro-1,4-dihydroquinazolin-2-yl)-N,N-dipropyl-3H-1-benzazepine-4-carboxamide,
2-amino-8-(4,4-dimethyl-1H-quinazolin-2-yl)-N,N-dipropyl-3H-1-benzazepine-4-carboxamide,
2-amino-8-(6-chloro-1,4-dihydroquinazolin-2-yl)-iV,iV-dipropyl-3H-1-benzazepine-4-carboxamide,
2-amino-8-(5-methyl-1,4-dihydroquinazolin-2-yl)-N,N-dipropyl-3H-1-benzazepine-4-carboxamide,
2-amino-8-(5-fluoro-1,4-dihydroquinazolin-2-yl)-N,N-dipropyl-3H-1-benzazepine-4-carboxamide, and
2-amino-8-(6-methoxy-1,4-dihydroquinazolin-2-yl)-N,N-dipropyl-3H-1-benzazepine-4-carboxamide.
   Aspect 13. A compound of formula K according to any one of Aspects 1 to 12 for use as medicament.
   Aspect 14. A compound of formula K according to any one of Aspects 1 to 12 for use as medicament for the treatment of diseases which can be mediated with TLR agonists.
   Aspect 15. A pharmaceutical composition comprising a compound of formula K according to any one of Aspects 1 to 12 and a pharmaceutically acceptable carrier and/or adjuvant.
   Aspect 16. The use of a compound of formula K according to any one of Aspects 1 to 12 for the preparation of a medicament for the treatment of diseases for the treatment of diseases which can be mediated with TLR agonists, particularly for the treatment of diseases selected from the group consisting of cancer, autoimmune diseases, inflammation, sepsis, allergy, asthma, graft rejection, graft-versus-host disease, immunodeficiencies, and infectious diseases.
   Aspect 17. A process for the manufacture of a compound of formula K as defined in Aspect 1, which process comprises
   a) coupling a compound of the formula II

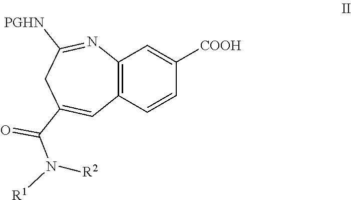

wherein R¹ and R² are as defined in Aspect 1 and PG is a protecting group, with compound of the formula III

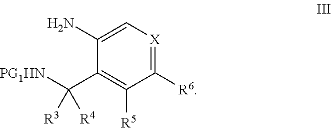

wherein X and R³, R⁴, R⁵ and R⁶ are as defined in Aspect 1 and PGi is a protecting group, under basic conditions in the presence of a coupling agent and removing the protecting groups PG and PG₁ under acidic conditions to obtain a compound of the formula K

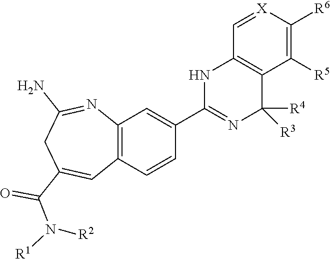

wherein X and R¹ to R⁶ are as defined in Aspect 1, and, if desired, converting the compound obtained into a pharmaceutically acceptable salt.

### Compounds J and I

Unless otherwise indicated, references to substituents (e.g., R¹), compounds, formulas, “Tables”, “Examples”, “Schemes”, and “Aspects” within this section, “Compounds J and I”, are intended to refer to such as defined within this section, “Compounds J and I”.

Set forth herein are diamino pyrido[3,2 D] pyrimidine compounds and pharmaceutical compositions which, among other things, may modulate toll-like receptors (e.g. TLR-8) and methods of making and using them.

The toll-like receptor (TLR) family plays a fundamental role in pathogen recognition and activation of innate immunity. Toll-like receptor 8 (TLR-8) is predominantly expressed by myeloid immune cells and activation of this receptor stimulates a broad immunological response. Agonists of TLR-8 activate myeloid dendritic cells, monocytes, monocyte-derived dendridic cells and Kupffer cells leading to the production of proinflammatory cytokines and chemokines, such as interleukin-18 (IL-18), interleukin-12 (IL-12), tumor necrosis factor-alpha (TNF-a), and interferon-gamma (IFN-γ). Such agonists also promote the increased expression of co-stimulatory molecules such as CD8⁺ cells, major histocompatibility complex molecules (MAIT, NK cells), and chemokine receptors.

Collectively, activation of these innate and adaptive immune responses induces an immune response and provides a therapeutic benefit in various conditions involving autoimmunity, inflammation, allergy, asthma, graft rejection, graft versus host disease (GvHD), infection, cancer, and immunodeficiency. For example, with respect to hepatitis B, activation of TLR8 on professional antigen presenting cells (pAPCs) and other intrahepatic immune cells is associated with induction of IL-12 and proinflammatory cytokines, which is expected to augment HBV-specific T cell responses, activate intrahepatic NK cells and drive reconstitution of antiviral immunity. See e.g. Wille-Reece, U. et al. J Exp Med 203, 1249-1258 (2006); Peng, G. et al, Science 309, 1380-1384 (2005); Jo, J. et al., PLoS Pathogens 10, e1004210 (2014) and Watashi, K. et al., J Biol Chem 288, 31715-31727 (2013).

Given the potential to treat a wide array of diseases, there remains a need for novel modulators of toll like receptors, for example TLR-8. Potent and selective modulators of TLR-8 that have reduced potential for off target liabilities are particularly desirable.

The present disclosure provides a compound of Formula (J):

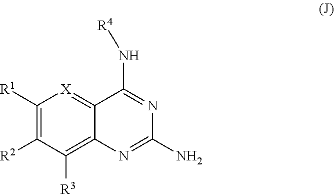

or a pharmaceutically acceptable salt thereof, wherein:

### X is N or CR¹⁰;

R¹ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups;
R² is selected from the group consisting of hydrogen, halogen, C₁₋₆alkyl, CN, —NR^{a}R^{b}, —S(O)₁₋₂R^{a} and OR^{a}, wherein C₁₋₆alkyl is optionally substituted with 1 to 5 R²⁰ groups;
R³ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups;
R⁴ is C₁₋₁₂ alkyl which is optionally substituted with 1 to 5 substituents independently selected from halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆haloalkyl, C₃₋₆cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur;
wherein each C₃₋₆cycloalkyl, 3 to 6 membered heterocyclyl, C₆₋₁₀ aryl, and 5 to 10 membered heteroaryl is optionally substituted with 1 to 5 R²¹ groups;
R¹⁰ is selected from hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups
each R²⁰ is independently selected from the group consisting of halogen, C₁₋₆haloalkyl, CN, —NR^{a}R^{b}, S(O)₁₋₂R^{a}, and OR^{a};
each R²¹ is independently selected from the group consisting of halogen, C₁₋₆alkyl, C₁₋₆haloalkyl, CN, —NR^{a}R^{b}, S(O)₁₋₂R^{a}, and OR^{a}; and
each R^{a} and R^{b} are independently selected from the group consisting of hydrogen and C₁₋₆alkyl; wherein each C₁₋₆alkyl is optionally substituted with 1 to 5 substituents independently selected from halogen, hydroxyl, amino, 5 to 10 membered heteroaryl wherein the 5 to 10 membered heteroaryl has 1 to 3 heteroatoms selected from oxygen, nitrogen, and sulfur, and C₁₋₆haloalkyl; provided that when X is N, R¹ is Cl, R² is H and R³ is H then R⁴ is not CH₂CH₂OMe or CH₂CH₂SO₂Me.

The present disclosure provides a compound of Formula (I):

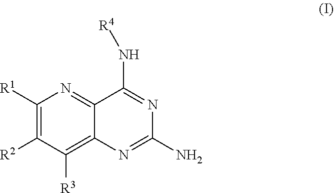

or a pharmaceutically acceptable salt thereof, wherein:
R¹ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups;
R² is selected from the group consisting of hydrogen, halogen, C₁₋₆alkyl, CN, —NR^{a}R^{b}, —S(O)₁₋₂R^{a} and OR^{a}, wherein C₁₋₆alkyl is optionally substituted with 1 to 5 R²⁰ groups;
R³ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups;
R⁴ is C₁₋₁₂ alkyl which is optionally substituted with 1 to 5 substituents independently selected from halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆haloalkyl, C₃₋₆cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur;
wherein each C₃₋₆cycloalkyl, 3 to 6 membered heterocyclyl, C₆₋₁₀ aryl, and 5 to 10 membered heteroaryl is optionally substituted with 1 to 5 R²¹ groups;
each R²⁰ is independently selected from the group consisting of halogen, C₁₋₆haloalkyl, CN, —NR^{a}R^{b}, S(O)₁₋₂R^{a}, and OR^{a};
each R²¹ is independently selected from the group consisting of halogen, C₁₋₆alkyl, C₁₋₆haloalkyl, CN, —NR^{a}R^{b}, S(O)₁₋₂R^{a}, and OR^{a}; and
each R^{a} and R^{b} are independently selected from the group consisting of hydrogen and C₁₋₆alkyl; wherein each C₁₋₆alkyl is optionally substituted with 1 to 5 substituents independently selected from halogen, hydroxyl, amino, 5 to 10 membered heteroaryl wherein the 5 to 10 membered heteroaryl has 1 to 3 heteroatoms selected from oxygen, nitrogen, and sulfur, and C₁₋₆haloalkyl; provided that when R¹ is Cl, R² is H and R³ is H then R⁴ is not CH₂CH₂OMe or CH₂CH₂SO₂Me.

The present disclosure provides a compound of Formula (IV):

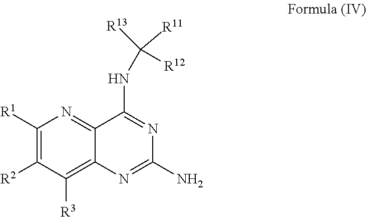

wherein:
R¹ is selected from the group consisting of hydrogen, halogen, C₁₋₆ alkyl, CN, and OR^{a}, wherein C₁₋₆alkyl is optionally substituted with 1 to 5 R²⁰ groups;
R² is selected from the group consisting of hydrogen, halogen, C₁₋₆ alkyl, CN, and OR^{a}, wherein C₁₋₆ alkyl optionally substituted with 1 to 5 R²⁰ groups; R³ is selected from the group consisting of hydrogen, halogen, C₁₋₆alkyl, CN, and OR^{a}, wherein C₁₋₆alkyl is optionally substituted with 1 to 5 R²⁰ groups;
R¹¹ is selected from the group consisting of C₁₋₂ alkyl, C₃₋₆ cycloalkyl, and C₁₋₃ haloalkyl;
R¹² is selected from C₁₋₃ alkyl, halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₃ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur, wherein the C₁₋₃ alkyl group is optionally substituted with 1 or 2 substituents independently selected from halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₃ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur;
R¹³ is selected from C₁₋₆ alkyl, halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur, wherein the C₁₋₆ alkyl is optionally substituted with 1 to 2 substituents independently selected from halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur; each R²⁰ is independently selected from the group consisting of halogen, CN, —NR^{a}R^{b}, and OR^{a}; and
each R^{a} and R^{b} is independently selected from the group consisting of hydrogen and C₁₋₃ alkyl, wherein each C₁₋₃ alkyl is optionally substituted with 1 to 3 substituents independently selected from halogen, —OH, and NH₂.

In certain embodiments, the present disclosure provides a pharmaceutical composition comprising a compound of the present disclosure, or a pharmaceutically acceptable salt thereof, and a pharmaceutically acceptable excipient. In certain embodiments, the pharmaceutical composition comprises one or more additional therapeutic agents.

In certain embodiments, a method of modulating TLR-8 is provided, comprising administering a compound of the present disclosure, or a pharmaceutically acceptable salt thereof, to an individual (e.g. a human).

In certain embodiments, a method of treating or preventing a disease or condition responsive to the modulation of TLR-8 is provided, comprising administering to an individual (e.g. a human) in need thereof a therapeutically effective amount of a compound of the present disclosure, or a pharmaceutically acceptable salt thereof. In certain embodiments, the method of treating or preventing a disease or condition responsive to the modulation of TLR-8, comprises administering one or more additional therapeutic agents.

In certain embodiments, a method of treating or preventing a viral infection is provided, comprising administering to an individual (e.g. a human) in need thereof a therapeutically effective amount a compound of the present disclosure, or a pharmaceutically acceptable salt thereof.

In certain embodiments, a method of treating or preventing a hepatitis B viral infection is provided, comprising administering to an individual (e.g. a human) in need thereof a therapeutically effective amount of a compound of the present disclosure, or a pharmaceutically acceptable salt thereof. In certain embodiments, the method of treating or preventing a hepatitis B viral infection comprises administering one or more additional therapeutic agents. In certain embodiments, the individual is a human infected with hepatitis B.

In certain embodiments, a method of treating or preventing a HIV infection is provided, comprising administering to an individual (e.g. a human) in thereof a therapeutically effective amount a compound of the present disclosure, or a pharmaceutically acceptable salt thereof. In certain embodiments, the method of treating or preventing a HIV infection comprises administering one or more additional therapeutic agents. In certain embodiments, the individual is a human infected with HIV (e.g. HIV-1).

In certain embodiments, a method of treating a hyperproliferative disease (e.g. cancer) is provided, comprising administering to an individual (e.g. a human) in thereof a therapeutically effective amount a compound of the present disclosure, or a pharmaceutically acceptable salt thereof. In certain embodiments, the method of treating a hyperproliferative disease (e.g. cancer) comprises administering one or more additional therapeutic agents. In certain embodiments, the individual is a human.

In certain embodiments, a compound of the present disclosure, or a pharmaceutically acceptable salt thereof, for use in medical therapy is provided.

In certain embodiments, a compound of the present disclosure, or a pharmaceutically acceptable salt thereof, for use in treating or preventing a disease or condition responsive to the modulation of TLR-8, is provided. In certain embodiments, the disease or condition is a viral infection.

In certain embodiments, a compound of the present disclosure, or a pharmaceutically acceptable salt thereof, for use in treating or preventing hepatitis B, is provided

In certain embodiments, the use of a compound of the present disclosure, or a pharmaceutically acceptable salt thereof, for the manufacture of a medicament for treating or preventing a disease or condition responsive to the modulation of TLR-8, is provided.

In certain embodiments, the use of a compound of the present disclosure, or a pharmaceutically acceptable salt thereof, for the manufacture of a medicament for treating or preventing hepatitis B, is provided.

Kits comprising the compounds, or pharmaceutically acceptable salts thereof, or pharmaceutical compositions of the foregoing are also provided. Articles of manufacture comprising a unit dose of the compounds, or pharmaceutically acceptable salts thereof, of the foregoing are also provided. Methods of preparing compounds of the present disclosure are also provided.

The description below is made with the understanding that the present disclosure is to be considered as an exemplification of subject matter, and is not intended to limit the appended Aspects to the specific embodiments illustrated. The headings used throughout this disclosure are provided for convenience and are not to be construed to limit the Aspects in any way. Embodiments illustrated under any heading may be combined with embodiments illustrated under any other heading.

Unless defined otherwise, all technical and scientific terms used herein have the same meaning as commonly understood by one of ordinary skill in the art. A dash at the front or end of a chemical group is a matter of convenience to indicate the point of attachment to a parent moiety; chemical groups may be depicted with or without one or more dashes without losing their ordinary meaning. A prefix such as “Cᵤ₋ᵥ” or (Cᵤ-Cᵥ) indicates that the following group has from u to v carbon atoms, where u and v are integers. For example, C₁₋₆alkyl indicates that the alkyl group has from 1 to 6 carbon atoms.

“Alkyl” is a linear or branched saturated monovalent hydrocarbon. For example, an alkyl group can have 1 to 10 carbon atoms (i.e., (C₁₋₁₀)alkyl) or 1 to 8 carbon atoms (i.e., (C₁₋₈)alkyl) or 1 to 6 carbon atoms (i.e., (C₁₋₆ alkyl) or 1 to 4 carbon atoms (i.e., (C₁₋₄)alkyl). Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl(—C(CH₃)₂CH₂CH₃), 3-methyl-2-butyl (—CH(CH₃)CH(CH₃)₂), 3-methyl-1-butyl(—CH₂CH₂CH(CH₃)₂), 2-methyl-1-butyl (—CH₂CH(CH₃)CH₂CH₃), 1-hexyl(—CH₂CH₂CH₂CH₂CH₂CH₃), 2-hexyl (—CH(CH₃)CH₂CH₂CH₂CH₃), 3-hexyl(—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl(—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, and octyl (—(CH₂)₇CH₃).

“Alkenyl” is a linear or branched monovalent hydrocarbon radical with at least one carbon-carbon double bond. For example, an alkenyl group can have 2 to 8 carbon atoms (i.e., C₂₋₈ alkenyl), or 2 to 6 carbon atoms (i.e., C₂₋₆ alkenyl) or 2 to 4 carbon atoms (i.e., C₂₋₄ alkenyl). Examples of suitable alkenyl groups include, but are not limited to, ethylene or vinyl (—CH═CH₂), allyl (—CH₂CH═CH₂), 5-hexenyl(—CH₂CH₂CH₂CH₂CH═CH₂), and 3-hexenyl (—CH₂CH₂CH═CHCH₂CH₂).

“Alkynyl” is a linear or branched monovalent hydrocarbon radical with at least one carbon-carbon triple bond. For example, an alkynyl group can have 2 to 8 carbon atoms (i.e., C₂₋₈ alkyne) or 2 to 6 carbon atoms (i.e., C₂₋₆ alkynyl) or 2 to 4 carbon atoms (i.e., C₂₋₄ alkynyl). Examples of alkynyl groups include, but are not limited to, acetylenyl (—C≡CH), propargyl (—CH2C≡CH), and —CH2-C≡C—CH3.

The term “halo” or “halogen” as used herein refers to fluoro (—F), chloro (—Cl), bromo (—Br) and iodo (—I).

The term “haloalkyl” as used herein refers to an alkyl as defined herein, wherein one or more hydrogen atoms of the alkyl are independently replaced by a halo substituent, which may be the same or different. For example, C₁₋₈haloalkyl is a C₁₋₈alkyl wherein one or more of the hydrogen atoms of the C₁₋₈alkyl have been replaced by a halo substituent. Examples of haloalkyl groups include but are not limited to fluoromethyl, fluorochloromethyl, difluoromethyl, difluorochloromethyl, trifluoromethyl, 1,1,1-trifluoroethyl and pentafluoroethyl.

The term “heteroalkyl” as used herein refers to an alkyl as defined herein, wherein one or more of the carbon atoms of the alkyl are replaced by an O, S, or NR^{q}, wherein each R^{q} is independently H or C₁₋₆alkyl. For example, C₁₋₈heteroalkyl intends a heteroalkyl of one to eight carbons wherein one or more carbon atoms is replaced by a heteroatom (e.g., O, S, NR^{q}, OH, SH or N(R^{q})₂), which may the same or different. Examples of heteroalkyls include but are not limited to methoxymethyl, ethoxymethyl, methoxy, 2-hydroxyethyl and N,N′-dimethylpropylamine. A heteroatom of a heteroalkyl may optionally be oxidized or alkylated. A heteroatom may be placed at any interior position of the heteroalkyl group or at a position at which the group is attached to the remainder of the molecule. Examples include, but are not limited to, —CH₂OCH₃, —CH₂CH₂NHCH₃, —CH₂CH₂N(CH₃)—CH₃, —CH₂SCH₂CH₃, —S(O)CH₃, —CH₂CH₂S(O)₂CH₃, —CHCHOCH₃, —CH₂CHNOCH₃, —CHCHN(CH₃)CH₃, —CH₂NHOCH₃ and —CH₂OS(CH₃)₃

The term “aryl” as used herein refers to a single all carbon aromatic ring or a multiple condensed all carbon ring system wherein at least one of the rings is aromatic. For example, in certain embodiments, an aryl group has 6 to 20 carbon atoms, 6 to 14 carbon atoms, or 6 to 12 carbon atoms. Aryl includes a phenyl radical. Aryl also includes multiple condensed ring systems (e.g., ring systems comprising 2, 3 or 4 rings) having about 9 to 20 carbon atoms in which at least one ring is aromatic and wherein the other rings may be aromatic or not aromatic (i.e., carbocycle). Such multiple condensed ring systems are optionally substituted with one or more (e.g., 1, 2 or 3) oxo groups on any carbocycle portion of the multiple condensed ring system. The rings of the multiple condensed ring system can be connected to each other via fused, spiro and bridged bonds when allowed by valency requirements. It is also to be understood that when reference is made to a certain atom-range membered aryl (e.g., 6-10 membered aryl), the atom range is for the total ring atoms of the aryl. For example, a 6-membered aryl would include phenyl and a 10-membered aryl would include naphthyl and 1, 2, 3, 4-tetrahydronaphthyl.

Non-limiting examples of aryl groups include, but are not limited to, phenyl, indenyl, naphthyl, 1, 2, 3, 4-tetrahydronaphthyl, anthracenyl, and the like.

The term “heteroaryl” as used herein refers to a single aromatic ring that has at least one atom other than carbon in the ring, wherein the atom is selected from the group consisting of oxygen, nitrogen and sulfur; “heteroaryl” also includes multiple condensed ring systems that have at least one such aromatic ring, which multiple condensed ring systems are further described below. Thus, “heteroaryl” includes single aromatic rings of from about 1 to 6 carbon atoms and about 1-4 heteroatoms selected from the group consisting of oxygen, nitrogen and sulfur. The sulfur and nitrogen atoms may also be present in an oxidized form provided the ring is aromatic. Exemplary heteroaryl ring systems include but are not limited to pyridyl, pyrimidinyl, oxazolyl or furyl. “Heteroaryl” also includes multiple condensed ring systems (e.g., ring systems comprising 2, 3 or 4 rings) wherein a heteroaryl group, as defined above, is condensed with one or more rings selected from heteroaryls (to form for example 1,8-naphthyridinyl), heterocycles, (to form for example 1,2,3,4-tetrahydro-1,8-naphthyridinyl), carbocycles (to form for example 5,6,7,8-tetrahydroquinolyl) and aryls (to form for example indazolyl) to form the multiple condensed ring system. Thus, a heteroaryl (a single aromatic ring or multiple condensed ring system) has about 1-20 carbon atoms and about 1-6 heteroatoms within the heteroaryl ring. Such multiple condensed ring systems may be optionally substituted with one or more (e.g., 1, 2, 3 or 4) oxo groups on the carbocycle or heterocycle portions of the condensed ring. The rings of the multiple condensed ring system can be connected to each other via fused, spiro and bridged bonds when allowed by valency requirements. It is to be understood that the individual rings of the multiple condensed ring system may be connected in any order relative to one another. It is to be understood that the point of attachment for a heteroaryl or heteroaryl multiple condensed ring system can be at any suitable atom of the heteroaryl or heteroaryl multiple condensed ring system including a carbon atom and a heteroatom (e.g., a nitrogen). It also to be understood that when a reference is made to a certain atom-range membered heteroaryl (e.g., a 5 to 10 membered heteroaryl), the atom range is for the total ring atoms of the heteroaryl and includes carbon atoms and heteroatoms. For example, a 5-membered heteroaryl would include a thiazolyl and a 10-membered heteroaryl would include a quinolinyl. Exemplary heteroaryls include but are not limited to pyridyl, pyrrolyl, pyrazinyl, pyrimidinyl, pyridazinyl, pyrazolyl, thienyl, indolyl, imidazolyl, oxazolyl, isoxazolyl, thiazolyl, furyl, oxadiazolyl, thiadiazolyl, quinolyl, isoquinolyl, benzothiazolyl, benzoxazolyl, indazolyl, quinoxalyl, quinazolyl, 5,6,7,8-tetrahydroisoquinolinyl benzofuranyl, benzimidazolyl, thianaphthenyl, pyrrolo[2,3-b]pyridinyl, quinazolinyl-4(3H)-one, triazolyl, 4,5,6,7-tetrahydro-1H-indazole and 3b,4,4a,5-tetrahydro-1H-cyclopropa[3,4]cyclopenta[1,2-c]pyrazole.

The term “cycloalkyl” refers to a single saturated or partially unsaturated all carbon ring having 3 to 20 annular carbon atoms (i.e., C₃₋₂₀ cycloalkyl), for example from 3 to 12 annular atoms, for example from 3 to 10 annular atoms. The term “cycloalkyl” also includes multiple condensed, saturated and partially unsaturated all carbon ring systems (e.g., ring systems comprising 2, 3 or 4 carbocyclic rings). Accordingly, cycloalkyl includes multicyclic carbocycles such as a bicyclic carbocycles (e.g., bicyclic carbocycles having about 6 to 12 annular carbon atoms such as bicyclo[3.1.0]hexane and bicyclo[2.1.1]hexane), and polycyclic carbocycles (e.g tricyclic and tetracyclic carbocycles with up to about 20 annular carbon atoms). The rings of a multiple condensed ring system can be connected to each other via fused, spiro and bridged bonds when allowed by valency requirements. Non-limiting examples of monocyclic cycloalkyl include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl and 1-cyclohex-3-enyl.

The term “heterocyclyl” or“heterocycle” as used herein refers to a single saturated or partially unsaturated non-aromatic ring or a non-aromatic multiple ring system that has at least one heteroatom in the ring (i.e., at least one annular heteroatom selected from oxygen, nitrogen, and sulfur). Unless otherwise specified, a heterocyclyl group has from 5 to about 20 annular atoms, for example from 3 to 12 annular atoms, for example from 5 to 10 annular atoms. Thus, the term includes single saturated or partially unsaturated rings (e.g., 3, 4, 5, 6 or 7-membered rings) having from about 1 to 6 annular carbon atoms and from about 1 to 3 annular heteroatoms selected from the group consisting of oxygen, nitrogen and sulfur in the ring. The rings of the multiple condensed ring system can be connected to each other via fused, spiro and bridged bonds when allowed by valency requirements. Heterocycles include, but are not limited to, azetidine, aziridine, imidazolidine, morpholine, oxirane (epoxide), oxetane, piperazine, piperidine, pyrazolidine, piperidine, pyrrolidine, pyrrolidinone, tetrahydrofuran, tetrahydrothiophene, dihydropyridine, tetrahydropyridine, quinuclidine, N-bromopyrrolidine, N-chloropiperidine, and the like.

The term “oxo” as used herein refers to ═O.

As used herein, “treatment” or “treating” is an approach for obtaining beneficial or desired results. For purposes of the present disclosure, beneficial or desired results include, but are not limited to, alleviation of a symptom and/or diminishment of the extent of a symptom and/or preventing a worsening of a symptom associated with a disease or condition. In one embodiment, “treatment” or“treating” includes one or more of the following: a) inhibiting the disease or condition (e.g., decreasing one or more symptoms resulting from the disease or condition, and/or diminishing the extent of the disease or condition); b) slowing or arresting the development of one or more symptoms associated with the disease or condition (e.g., stabilizing the disease or condition, delaying the worsening or progression of the disease or condition); and c) relieving the disease or condition, e.g., causing the regression of clinical symptoms, ameliorating the disease state, delaying the progression of the disease, increasing the quality of life, and/or prolonging survival.

A “compound of the present disclosure” includes compounds disclosed herein, for example a compound of the present disclosure includes compounds of Formula (J), (I), (Ia), (Ib), (II), (IIa), (IIb), (III), (IIIa), (IIIb), and the compounds listed in Table 1. A compound of the present disclosure also includes compounds of Formula (J), (I), (Ia), (Ib), (II), (IIa), (IIb), (III), (IIIa), (IIIb), (VI), (Va), (IVb), (IVc), (IVd), the compounds of Examples 1-113, and the compounds listed in Tables 1 and 3. A compound of the present disclosure also includes the compounds of Examples 1-118

As used herein, “delaying” development of a disease or condition means to defer, hinder, slow, retard, stabilize and/or postpone development of the disease or condition. This delay can be of varying lengths of time, depending on the history of the disease and/or individual being treated. As is evident to one skilled in the art, a sufficient or significant delay can, in effect, encompass prevention, in that the individual does not develop the disease or condition. For example, a method that“delays” development of AIDS is a method that reduces the probability of disease development in a given time frame and/or reduces extent of the disease in a given time frame, when compared to not using the method. Such comparisons may be based on clinical studies, using a statistically significant number of subjects. For example, the development of AIDS can be detected using known methods, such as confirming an individual's HIV⁺ status and assessing the individual's T-cell count or other indication of AIDS development, such as extreme fatigue, weight loss, persistent diarrhea, high fever, swollen lymph nodes in the neck, armpits or groin, or presence of an opportunistic condition that is known to be associated with AIDS (e.g., a condition that is generally not present in individuals with functioning immune systems but does occur in AIDS patients). Development may also refer to disease progression that may be initially undetectable and includes occurrence, recurrence and onset.

As used herein, “prevention” or “preventing” refers to a regimen that protects against the onset of the disease or disorder such that the clinical symptoms of the disease do not develop. Thus, “prevention” relates to administration of a therapy (e.g., administration of a therapeutic substance) to a subject before signs of the disease are detectable in the subject (e.g., administration of a therapeutic substance to a subject in the absence of detectable infectious agent (e.g., virus) in the subject). The subject may be an individual at risk of developing the disease or disorder, such as an individual who has one or more risk factors known to be associated with development or onset of the disease or disorder. Thus, in certain embodiments, the term “preventing HBV infection” refers to administering to a subject who does not have a detectable HBV infection an anti-HBV therapeutic substance. It is understood that the subject for anti-HBV preventative therapy may be an individual at risk of contracting the HBV virus. Thus, in certain embodiments, the term “preventing HIV infection” refers to administering to a subject who does not have a detectable HIV infection an anti-HIV therapeutic substance. It is understood that the subject for anti-HIV preventative therapy may be an individual at risk of contracting the HIV virus.

As used herein, an “at risk” individual is an individual who is at risk of developing a condition to be treated. An individual“at risk” may or may not have detectable disease or condition, and may or may not have displayed detectable disease prior to the treatment of methods described herein. “At risk” denotes that an individual has one or more so-called risk factors, which are measurable parameters that correlate with development of a disease or condition and are known in the art. An individual having one or more of these risk factors has a higher probability of developing the disease or condition than an individual without these risk factor(s). For example, individuals at risk for AIDS are those having HIV.

As used herein, the term “therapeutically effective amount” or “effective amount” refers to an amount that is effective to elicit the desired biological or medical response, including the amount of a compound that, when administered to a subject for treating a disease, is sufficient to effect such treatment for the disease. The effective amount will vary depending on the compound, the disease, and its severity and the age, weight, etc., of the subject to be treated. The effective amount can include a range of amounts. As is understood in the art, an effective amount may be in one or more doses, i.e., a single dose or multiple doses may be required to achieve the desired treatment endpoint. An effective amount may be considered in the context of administering one or more therapeutic agents, and a single agent may be considered to be given in an effective amount if, in conjunction with one or more other agents, a desirable or beneficial result may be or is achieved. Suitable doses of any co-administered compounds may optionally be lowered due to the combined action (e.g., additive or synergistic effects) of the compounds.

As used herein, an “agonist” is a substance that stimulates its binding partner, typically a receptor. Stimulation is defined in the context of the particular assay, or may be apparent in the literature from a discussion herein that makes a comparison to a factor or substance that is accepted as an “agonist” or an “antagonist” of the particular binding partner under substantially similar circumstances as appreciated by those of skill in the art. Stimulation may be defined with respect to an increase in a particular effect or function that is induced by interaction of the agonist or partial agonist with a binding partner and can include allosteric effects.

As used herein, an “antagonist” is a substance that inhibits its binding partner, typically a receptor. Inhibition is defined in the context of the particular assay, or may be apparent in the literature from a discussion herein that makes a comparison to a factor or substance that is accepted as an “agonist” or an “antagonist” of the particular binding partner under substantially similar circumstances as appreciated by those of skill in the art. Inhibition may be defined with respect to a decrease in a particular effect or function that is induced by interaction of the antagonist with a binding partner, and can include allosteric effects.

As used herein, a “partial agonist” or a “partial antagonist” is a substance that provides a level of stimulation or inhibition, respectively, to its binding partner that is not fully or completely agonistic or antagonistic, respectively. It will be recognized that stimulation, and hence, inhibition is defined intrinsically for any substance or category of substances to be defined as agonists, antagonists, or partial agonists.

As used herein, “intrinsic activity” or “efficacy” relates to some measure of biological effectiveness of the binding partner complex. With regard to receptor pharmacology, the context in which intrinsic activity or efficacy should be defined will depend on the context of the binding partner (e.g., receptor/ligand) complex and the consideration of an activity relevant to a particular biological outcome. For example, in some circumstances, intrinsic activity may vary depending on the particular second messenger system involved. Where such contextually specific evaluations are relevant, and how they might be relevant in the context of the present disclosure, will be apparent to one of ordinary skill in the art.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals

As used herein, modulation of a receptor includes agonism, partial agonism, antagonism, partial antagonism, or inverse agonism of a receptor.

The nomenclature used herein to name the subject compounds is illustrated in the Examples and elsewhere herein.

As used herein, “co-administration” includes administration of unit dosages of the compounds disclosed herein before or after administration of unit dosages of one or more additional therapeutic agents, for example, administration of the compound disclosed herein within seconds, minutes, or hours of the administration of one or more additional therapeutic agents. For example, in some embodiments, a unit dose of a compound of the present disclosure is administered first, followed within seconds or minutes by administration of a unit dose of one or more additional therapeutic agents. Alternatively, in other embodiments, a unit dose of one or more additional therapeutic agents is administered first, followed by administration of a unit dose of a compound of the present disclosure within seconds or minutes. In some embodiments, a unit dose of a compound of the present disclosure is administered first, followed, after a period of hours (e.g., 1-12 hours), by administration of a unit dose of one or more additional therapeutic agents. In other embodiments, a unit dose of one or more additional therapeutic agents is administered first, followed, after a period of hours (e.g., 1-12 hours), by administration of a unit dose of a compound of the present disclosure.

Provided are also pharmaceutically acceptable salts, hydrates, solvates, tautomeric forms, polymorphs, and prodrugs of the compounds described herein. “Pharmaceutically acceptable” or “physiologically acceptable” refer to compounds, salts, compositions, dosage forms and other materials which are useful in preparing a pharmaceutical composition that is suitable for veterinary or human pharmaceutical use.

The compounds of described herein may be prepared and/or formulated as pharmaceutically acceptable salts. Pharmaceutically acceptable salts are non-toxic salts of a free base form of a compound that possesses the desired pharmacological activity of the free base. These salts may be derived from inorganic or organic acids or bases. For example, a compound that contains a basic nitrogen may be prepared as a pharmaceutically acceptable salt by contacting the compound with an inorganic or organic acid. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington: The Science and Practice of Pharmacy, 21^{st} Edition, Lippincott Williams and Wilkins, Philadelphia, Pa., 2006.

Examples of “pharmaceutically acceptable salts” of the compounds disclosed herein also include salts derived from an appropriate base, such as an alkali metal (for example, sodium, potassium), an alkaline earth metal (for example, magnesium), ammonium and NX₄⁺ (wherein X is C₁-C₄ alkyl). Also included are base addition salts, such as sodium or potassium salts.

Provided are also compounds described herein or pharmaceutically acceptable salts, isomers, or a mixture thereof, in which from 1 to n hydrogen atoms attached to a carbon atom may be replaced by a deuterium atom or D, in which n is the number of hydrogen atoms in the molecule. As known in the art, the deuterium atom is a non-radioactive isotope of the hydrogen atom. Such compounds may increase resistance to metabolism, and thus may be useful for increasing the half-life of the compounds described herein or pharmaceutically acceptable salts, isomer, or a mixture thereof when administered to a mammal. See, e.g., Foster, “Deuterium Isotope Effects in Studies of Drug Metabolism”, Trends Pharmacol. Sci., 5(12):524-527 (1984). Such compounds are synthesized by means well known in the art, for example by employing starting materials in which one or more hydrogen atoms have been replaced by deuterium.

Examples of isotopes that can be incorporated into the disclosed compounds also include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of Formula (I), can generally be prepared by conventional techniques known to those skilled in the art or by processes analogous to those described in the Examples as set out below using an appropriate isotopically-labeled reagent in place of the non-labeled reagent previously employed.

The compounds of the embodiments disclosed herein, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centres of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present disclosure contemplates various stereoisomers and mixtures thereof and includes“enantiomers”, which refers to two stereoisomers whose molecules are non-superimposable mirror images of one another.

A “tautomer” refers to a proton shift from one atom of a molecule to another atom of the same molecule. The present disclosure includes tautomers of any said compounds.

A “solvate” is formed by the interaction of a solvent and a compound. Solvates of salts of the compounds described herein are also provided. Hydrates of the compounds described herein are also provided.

A “prodrug” includes any compound that becomes a compound described herein when administered to a subject, e.g., upon metabolic processing of the prodrug.

The terms“combination antiretroviral therapy” (“cART”) refers to combinations or “cocktails” of antiretroviral medications used to treat human viral infections, including HIV infections. As used herein, the terms “combination antiretroviral therapy” and “cART include combinations and regimens often referred to as Highly Active Antiretroviral Therapy (HAART). HAART and cART combinations and regimens commonly include multiple, often two or more, drugs such as nucleoside reverse transcriptase inhibitors (NRTIs), non-nucleoside reverse transcriptase inhibitors (NNRTIs), protease inhibitors (PIs), fusion inhibitors, CCR5 agonists, and/or integrase inhibitors.

The terms “latent HIV reservoir”, “HIV latent reservoir”, “HIV reservoir”, “latent reservoir”, and “latent HIV infection” refer to a condition in which resting CD4+ T lymphocytes or other cells are infected with HIV but are not actively producing HIV. The presently inactive HIV infected cells are referred to as “latently infected cells”. Antiretroviral therapy (ART) can reduce the level of HIV in the blood to an undetectable level, while latent reservoirs of HIV continue to survive. When a latently infected cell is reactivated, the cell begins to produce HIV (HIV replication).

The present disclosure provides a compound of Formula (J):

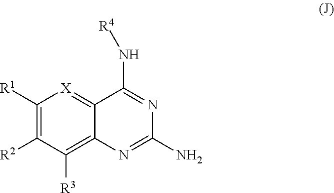

or a pharmaceutically acceptable salt thereof, wherein:

### X is N or CR¹⁰;

R¹ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups;
R² is selected from the group consisting of hydrogen, halogen, C₁₋₆alkyl, CN, —NR^{a}R^{b}, —S(O)₁₋₂R^{a} and OR^{a}, wherein C₁₋₆alkyl is optionally substituted with 1 to 5 R²⁰ groups;
R³ is selected from the group consisting of hydrogen, halogen, C1-6alkyl, CN, —NR^{a}R^{b}, —S(O)₁₋₂R^{a}, and OR^{a}, wherein C₁₋₆alkyl is optionally substituted with 1 to 5 R²⁰ groups;
R⁴ is C₁₋₁₂ alkyl which is optionally substituted with 1 to 5 substituents independently selected from halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆haloalkyl, C₃₋₆cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur;
wherein each C₃₋₆cycloalkyl, 3 to 6 membered heterocyclyl, C₆₋₁₀ aryl, and 5 to 10 membered heteroaryl is optionally substituted with 1 to 5 R²¹ groups;
R¹⁰ is selected from hydrogen, halogen, C₁₋₆alkyl, CN, —NR^{a}R^{b}, —S(O)₁₋₂R^{a} and OR^{a}, wherein C₁₋₆alkyl is optionally substituted with 1 to 5 R²⁰ groups each R²⁰ is independently selected from the group consisting of halogen, C₁₋₆haloalkyl, CN, —NR^{a}R^{b}, S(O)₁₋₂R^{a}, and OR^{a};
each R²¹ is independently selected from the group consisting of halogen, C₁₋₆alkyl, C₁₋₆haloalkyl, CN, —NR^{a}R^{b}, S(O)₁₋₂R^{a}, and OR^{a}; and each R^{a} and R^{b} are independently selected from the group consisting of hydrogen and C₁₋₆alkyl; wherein each C₁₋₆alkyl is optionally substituted with 1 to 5 substituents independently selected from halogen, hydroxyl, amino, 5 to 10 membered heteroaryl wherein the 5 to 10 membered heteroaryl has 1 to 3 heteroatoms selected from oxygen, nitrogen, and sulfur, and C₁₋₆haloalkyl;
provided that when X is N, R¹ is Cl, R² is H and R³ is H then R⁴ is not CH₂CH₂OMe or CH₂CH₂SO₂Me.

In certain embodiments of Formula (J), X is CR¹⁰. In certain embodiments of Formula (J), X is N.

The present disclosure provides a compound of Formula (I):

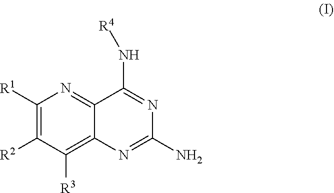

or a pharmaceutically acceptable salt thereof, wherein:
R¹ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups;
R² is selected from the group consisting of hydrogen, halogen, C₁₋₆alkyl, CN, —NR^{a}R^{b}, —S(O)₁₋₂R^{a} and OR^{a}, wherein C₁₋₆alkyl is optionally substituted with 1 to 5 R²⁰ groups;
R³ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups;
R⁴ is C₁₋₁₂ alkyl which is optionally substituted with 1 to 5 substituents independently selected from halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆haloalkyl, C₃₋₆cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur;
wherein each C₃₋₆cycloalkyl, 3 to 6 membered heterocyclyl, C₆₋₁₀ aryl, and 5 to 10 membered heteroaryl is optionally substituted with 1 to 5 R²¹ groups;
each R²⁰ is independently selected from the group consisting of halogen, C₁₋₆haloalkyl, CN, —NR^{a}R^{b}, S(O)₁₋₂R^{a}, and OR^{a};
each R²¹ is independently selected from the group consisting of halogen, C₁₋₆alkyl, C₁₋₆haloalkyl, CN, —NR^{a}R^{b}, S(O)₁₋₂R^{a}, and OR^{a}; and
each R^{a} and R^{b} are independently selected from the group consisting of H and C₁₋₆alkyl; wherein each C₁₋₆alkyl is optionally substituted with 1 to 5 substituents independently selected from halogen, hydroxyl, amino, 5 to 10 membered heteroaryl wherein the 5 to 10 membered heteroaryl has 1 to 3 heteroatoms selected from oxygen, nitrogen, and sulfur, and C₁₋₆haloalkyl; provided that when R¹ is Cl, R² is H and R³ is H then R⁴ is not CH₂CH₂OMe or CH₂CH₂SO₂Me.

In certain embodiments of a compound of Formula (J) or (I), R⁴ is C₁₋₈ alkyl which is optionally substituted with 1 to 5 substituents independently selected from the group consisting of halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆haloalkyl, C₃₋₆cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur; and wherein each C₃₋₆cycloalkyl, 3 to 6 membered heterocyclyl, C₆₋₁₀ aryl, and 5 to 10 membered heteroaryl is optionally substituted with 1 to 5 R²¹ groups.

In certain embodiments of a compound of Formula (J) or (I), R⁴ is C₁₋₆ alkyl optionally substituted with 1 to 5 substituents independently selected from the group consisting of halogen, —OR^{a}, —C(O)OR^{a}, —C(O)NR^{a}R^{b}, —SR^{a}, C₁₋₆haloalkyl, C₃₋₆cycloalkyl, 3 to 6 membered heterocyclyl, and C₆₋₁₀ aryl; wherein each C₃₋₆cycloalkyl, 3 to 6 membered heterocyclyl, and C₆₋₁₀ aryl is optionally substituted with 1 to 5 R²¹ groups. In certain embodiments of a compound of Formula (J) or (I), R⁴ is C₃₋₈ alkyl optionally substituted with 1 to 5 substituents independently selected from the group consisting of halogen, —OR^{a}, —C(O)OR^{a}, —NR^{a}C(O)R^{b}, —SR^{a}, C₁₋₆haloalkyl, C₃₋₆cycloalkyl, 3 to 6 membered heterocyclyl, and C₆₋₁₀ aryl; wherein each C₃₋₆cycloalkyl, 3 to 6 membered heterocyclyl, and C₆₋₁₀ aryl is optionally substituted with 1 to 5 R²¹ groups.

In certain embodiments of a compound of Formula (J) or (I), R⁴ is C₁₋₆ alkyl optionally substituted with 1 to 3 substituents independently selected from the group consisting of halogen, —OR^{a}, —C(O)OR^{a}, —C(O)NR^{a}R^{b}, —SR^{a}, —C₁₋₃haloalkyl, C₃₋₆cycloalkyl, 3 to 6 membered heterocyclyl and C₆₋₁₀ aryl; wherein each C₃₋₆cycloalkyl and C₆₋₁₀ aryl is optionally substituted with 1 to 3 R²¹ groups. In certain embodiments of a compound of Formula (J) or (I), R⁴ is C₃₋₈ alkyl optionally substituted with 1 to 3 substituents independently selected from the group consisting of halogen, —OR^{a}, —C(O)OR^{a}, —NR^{a}C(O)R^{b}, —SR^{a}, —C₁₋₃haloalkyl, C₃₋₆cycloalkyl, 3 to 6 membered heterocyclyl and C₆₋₁₀ aryl; wherein each C₃₋₆cycloalkyl and C₆₋₁₀ aryl is optionally substituted with 1 to 3 R²¹ groups.

In certain embodiments of a compound of Formula (J) or (I), R⁴ is C₁₋₆ alkyl optionally substituted with 1 or 2 substituents independently selected halogen, —OR^{a}, —C(O)OR^{a}, —C(O)NR^{a}R^{b}, —SR^{a}, C₁₋₃haloalkyl, C₃₋₆cycloalkyl, 3 to 6 membered heterocyclyl and C₆₋₁₀ aryl; wherein each C₃₋₆cycloalkyl and C₆₋₁₀ aryl is optionally substituted with 1 to 3 R²¹ groups and wherein R^{a} and R^{b} are each independently hydrogen or C₁₋₄alkyl, wherein the C₁₋₄ alkyl is optionally substituted with —NH₂, OH, or pyridyl. In certain embodiments of a compound of Formula (J) or (I), R⁴ is C₃₋₈ alkyl which is optionally substituted with 1 or 2 substituents independently selected from the group consisting of halogen, —OR^{a}, —C(O)OR^{a}, —NR^{a}C(O)R^{b}, —SR^{a}, C₁₋₃haloalkyl, C₃₋₆cycloalkyl, 3 to 6 membered heterocyclyl and C₆₋₁₀ aryl; wherein each C₃₋₆cycloalkyl and C₆₋₁₀ aryl is optionally substituted with 1 to 3 R²⁰ groups and wherein R^{a} and R^{b} are each independently hydrogen or C₁₋₄alkyl, wherein each C₁₋₄ alkyl is optionally substituted with —NH₂, OH, or pyridyl.

In certain embodiments of a compound of Formula (J) or (I), R⁴ is C₁₋₆ alkyl optionally substituted with 1 or 2 substituents independently selected from the group consisting of OH, CF₃, —C(O)OH, —C(O)OCH₃, —C(O)NH₂, SCH₃, —C(O)NHCH₃, —C(O)NHCH₂CH₂NH₂, —C(O)NHCH₂CH₂OH, —C(O)NHCH₂-pyridyl, phenyl, tetrahydrofuranyl, and cyclopropyl. In certain embodiments of a compound of Formula (J) or (I), R⁴ is C₃₋₈ alkyl which is optionally substituted with 1 or 2 substituents independently selected from OH, CF₃, —C(O)OH, —C(O)OCH₃, SCH₃, —NHC(O)CH₃, —NHC(O)CH₂CH₂NH₂, —NHC(O)CH₂CH₂OH, —NHC(O)CH₂-pyridyl, phenyl, tetrahydrofuranyl, and cyclopropyl.

In certain embodiments of a compound of Formula (J) or (I), R⁴ is C₃₋₆ alkyl optionally substituted with 1 or 2 substituents independently selected from the group consisting of OH, CF₃, —C(O)OH, —C(O)OCH₃, —C(O)NH₂, SCH₃, —C(O)NHCH₃, —C(O)NHCH₂CH₂NH₂, —C(O)NHCH₂CH₂OH, and —C(O)NHCH₂-pyridyl. In certain embodiments of a compound of Formula (J) or (I), R⁴ is C₃₋₆ alkyl which is optionally substituted with 1 or 2 substituents independently selected from OH, CF₃, —C(O)OH, —C(O)OCH₃, SCH₃, —NHC(O)CH₃, —NHC(O)CH₂CH₂NH₂, —NHC(O)CH₂CH₂OH, —NHC(O)CH₂-pyridyl, phenyl, tetrahydrofuranyl, and cyclopropyl.

In certain embodiments of a compound of Formula (J) or (I), R⁴ is C₁₋₆ alkyl which is optionally substituted with OH. In certain embodiments of a compound of Formula (J) or (I), R⁴ is C₃₋₈ alkyl which is optionally substituted with OH. In certain embodiments of a compound of Formula (J) or (I), R⁴ is C₃₋₈ alkyl which is substituted with —NHC(O)CH₃.

In certain embodiments of a compound of Formula (J) or (I), R⁴ is C₃₋₆ alkyl which is optionally substituted with OH. In certain embodiments of a compound of Formula (J) or (I), R⁴ is C₃₋₆ alkyl which is substituted with —NHC(O)CH₃.

In certain embodiments of a compound of Formula (J) or (I), R⁴ has at least one chiral center. In certain embodiments, the at least one chiral center is in the S configuration. In certain embodiments, the at least one chiral center is in the R configuration.

In certain embodiments of a compound of Formula (J) or (I), R⁴ is selected from the group consisting of:

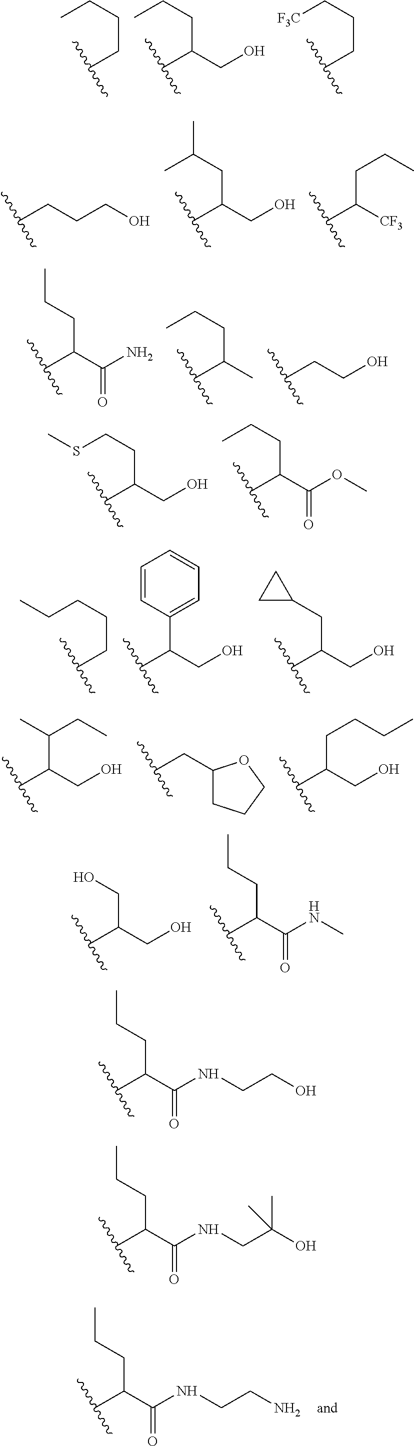

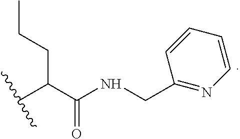 [SMILES: *C(CCC)C(=O)NCc1ccccn1]

In certain embodiments of a compound of Formula (J) or (I), R⁴ is selected from the group consisting of:

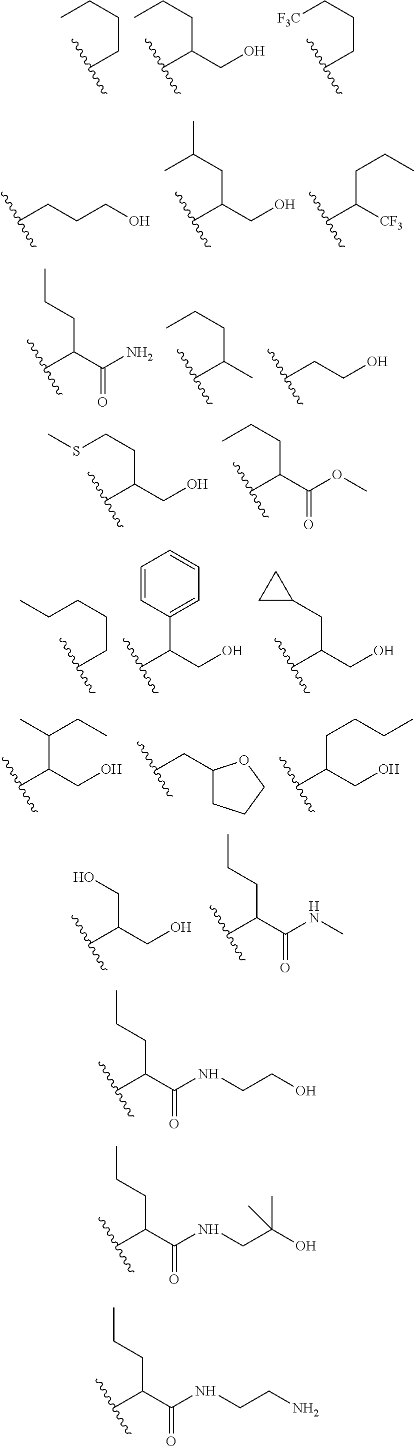

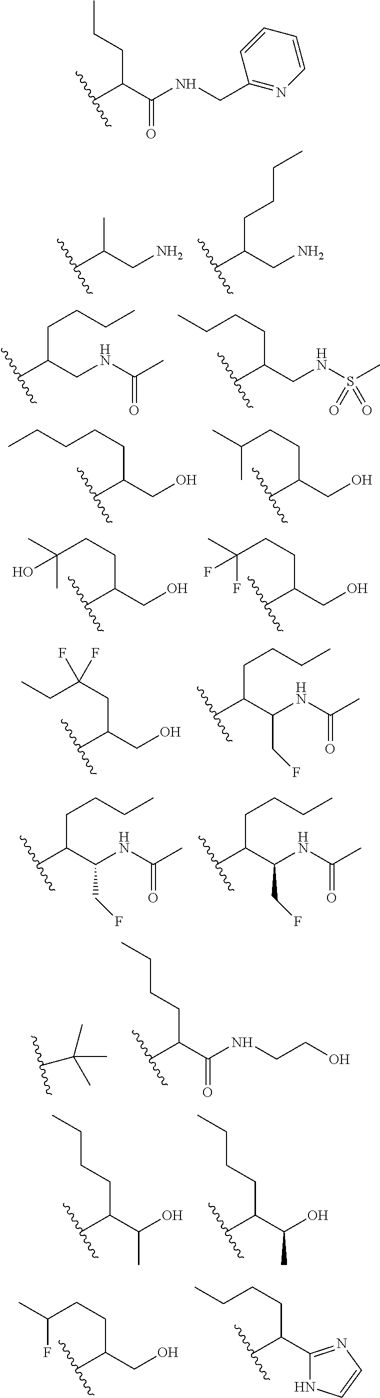

In certain embodiments of a compound of Formula (J) or (I), R⁴ is selected from the group consisting of:

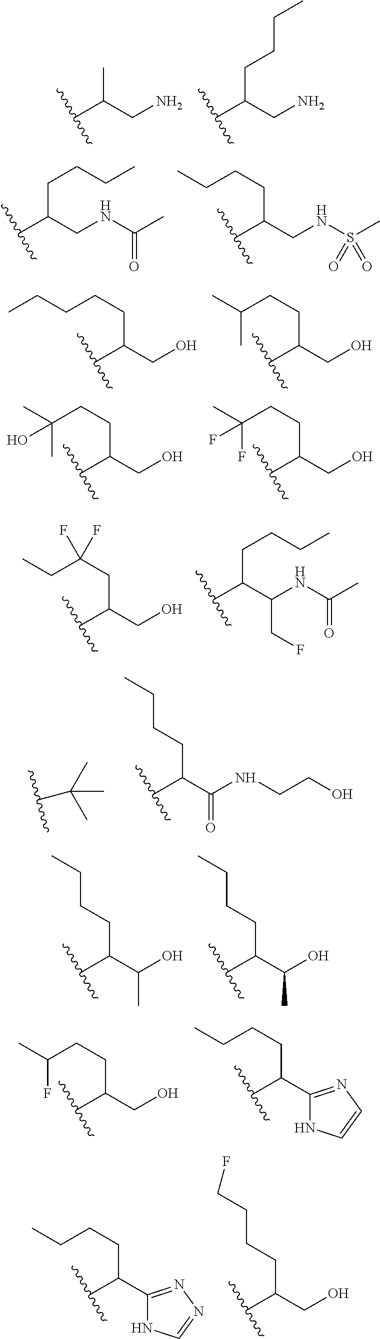

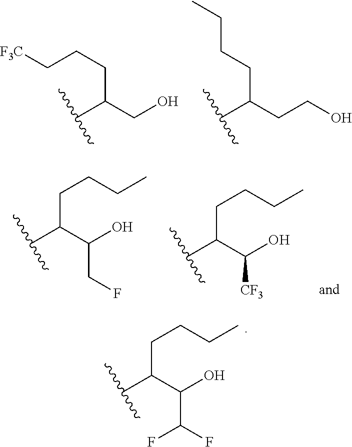

In certain embodiments of a compound of Formula (J) or (I), R⁴ is selected from the group consisting of:

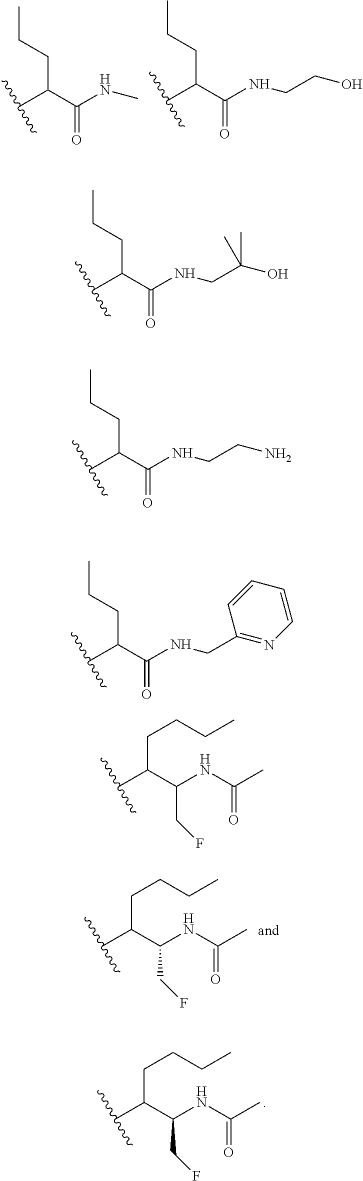

In certain embodiments of a compound of Formula (J) or (I), R⁴ is selected from the group consisting of:

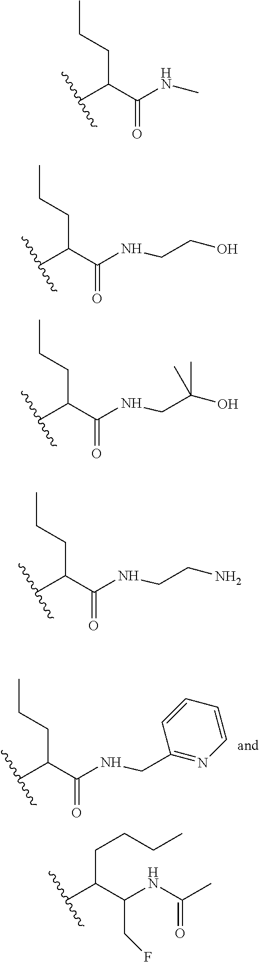

In certain embodiments of a compound of Formula (J) or (I), R⁴ is selected from the group consisting of:

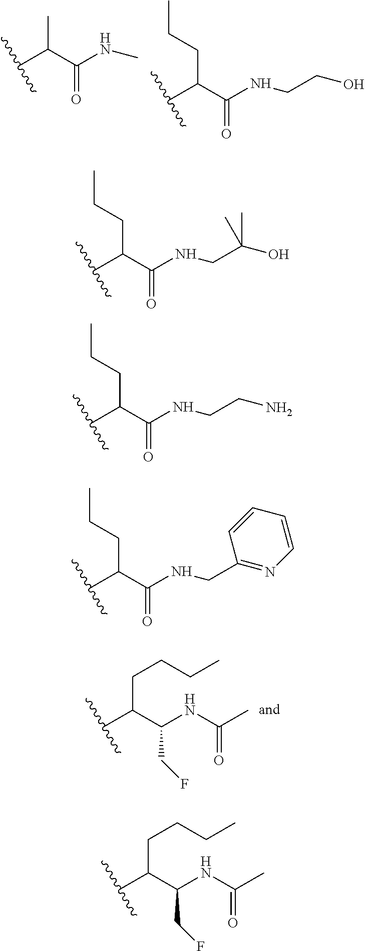

In certain embodiments of a compound of Formula (J) or (I), R⁴ is selected from the group consisting of:

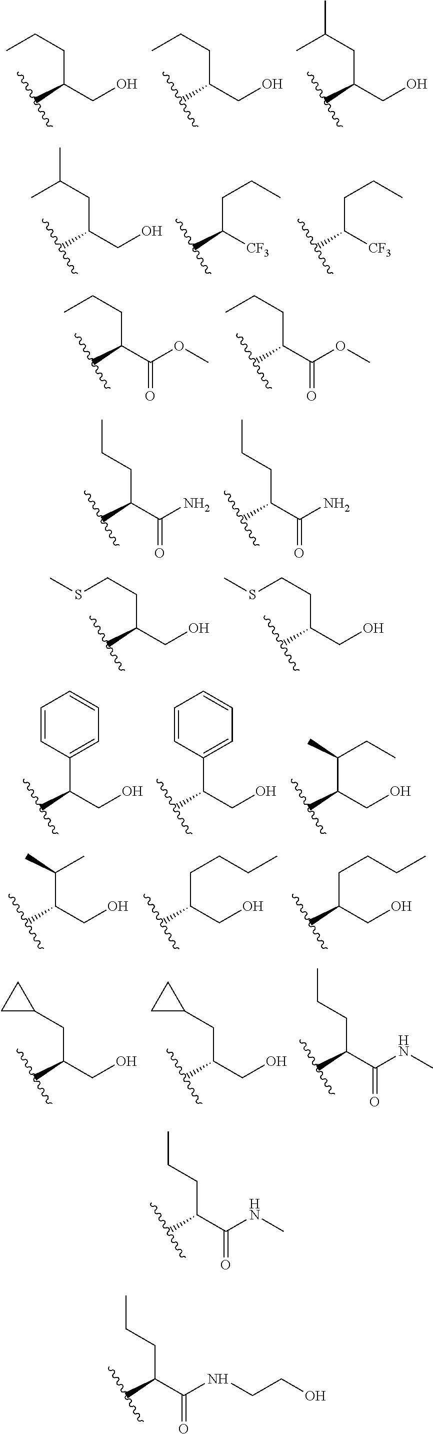

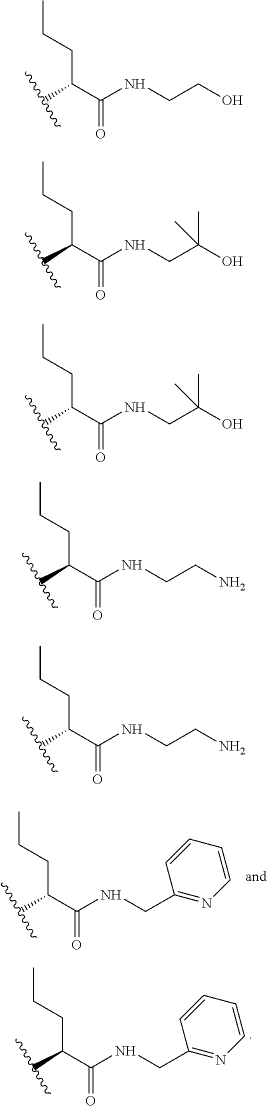

In certain embodiments of a compound of Formula (J) or (I), R⁴ is selected from the group consisting of:

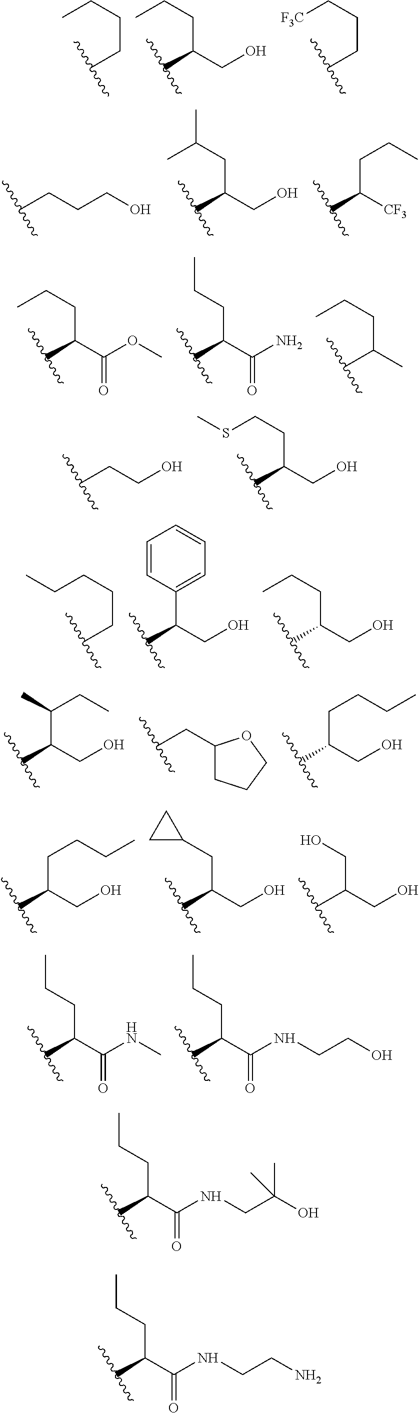

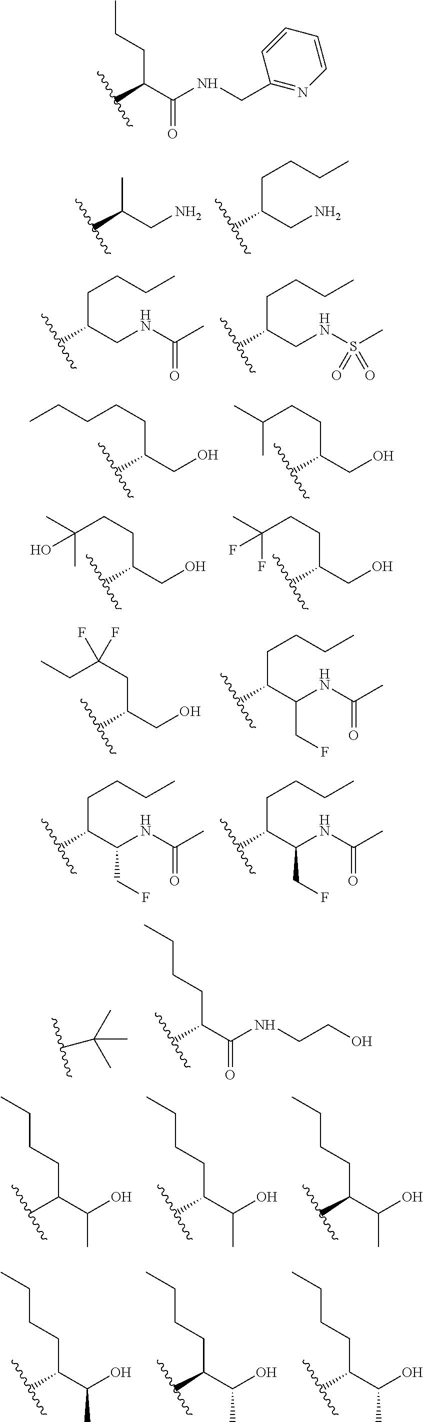

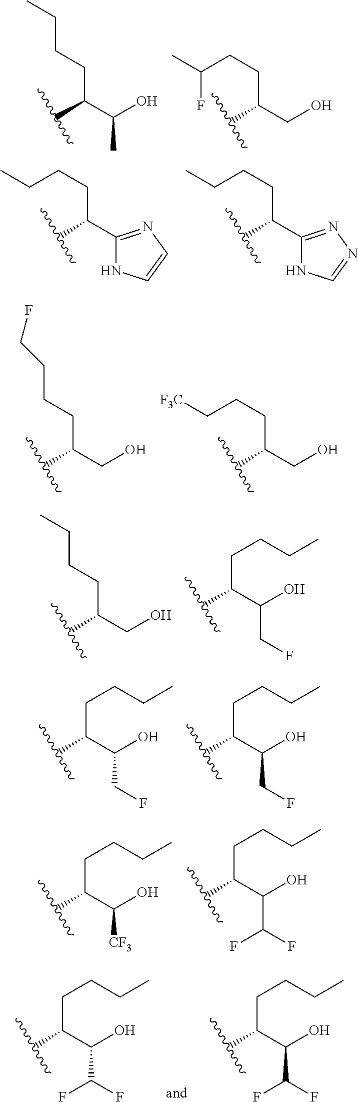

In certain embodiments of a compound of Formula (J) or (I), R⁴ is selected from the group consisting of:

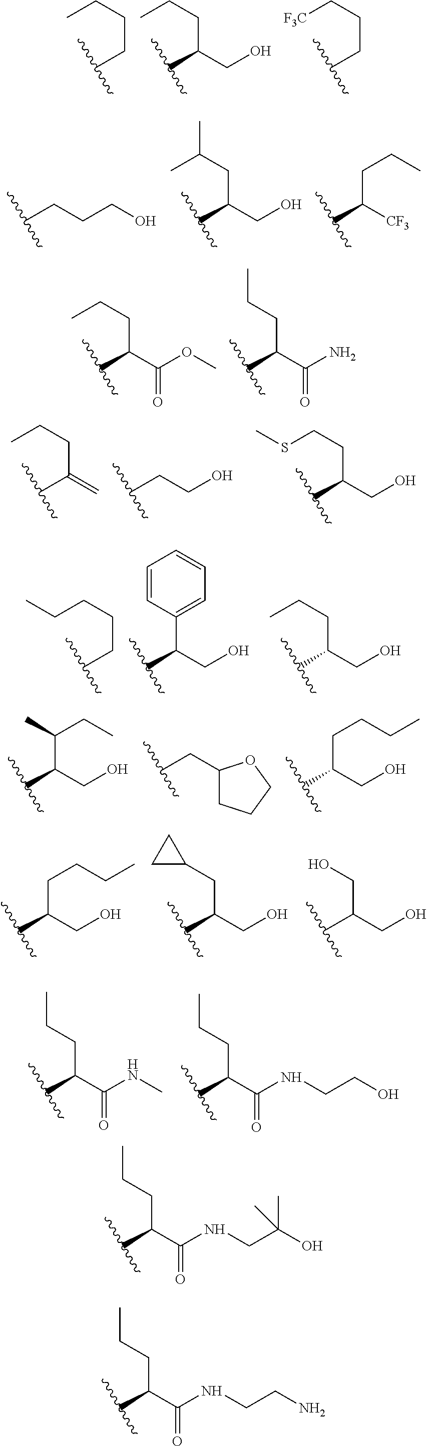

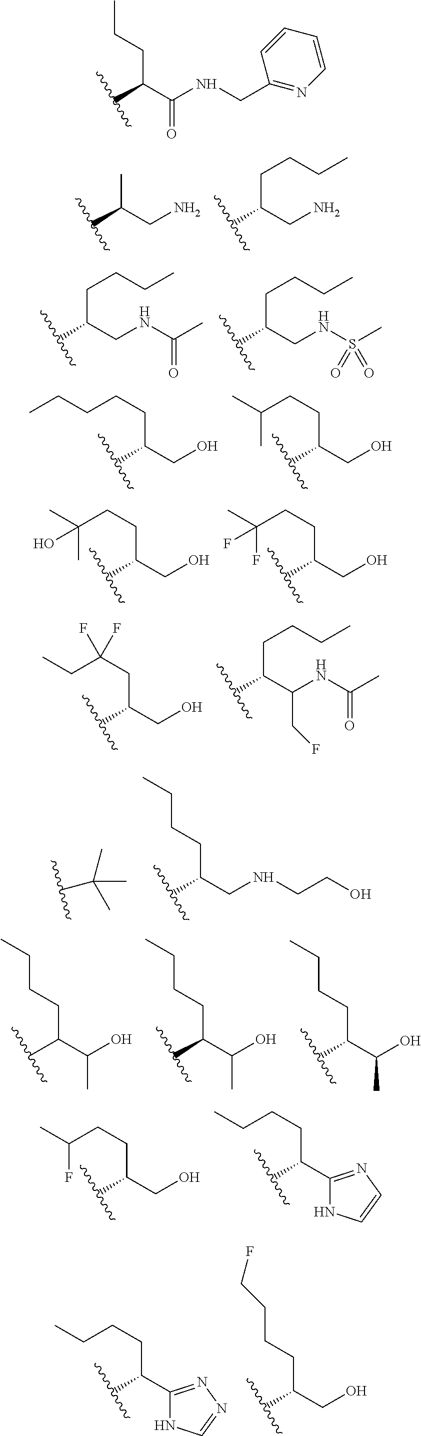

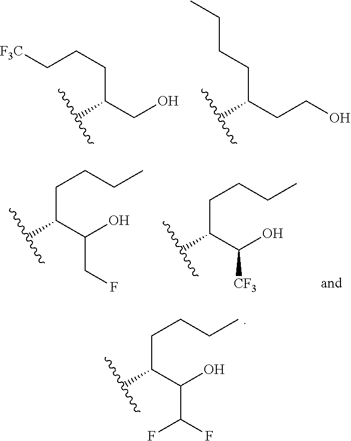

In certain embodiments of a compound of Formula (J) or (I), R⁴ is selected from the group consisting of:

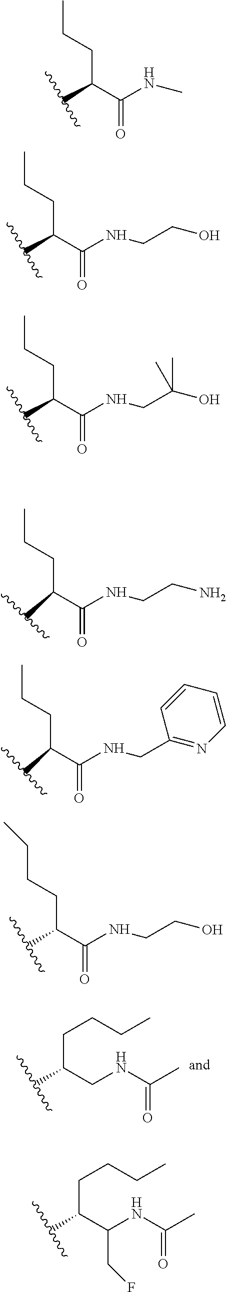

In certain embodiments of a compound of Formula (J) or (I), R⁴ is selected from the group consisting of:

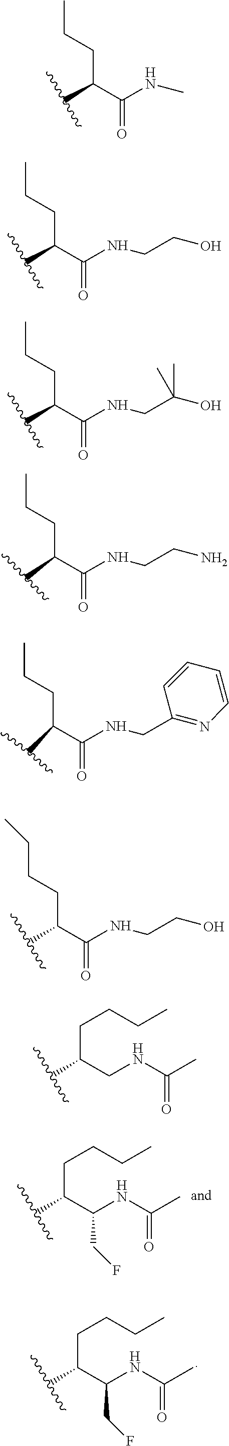

In certain embodiments of a compound of Formula (J) or (I), R⁴ is selected from the group consisting of:

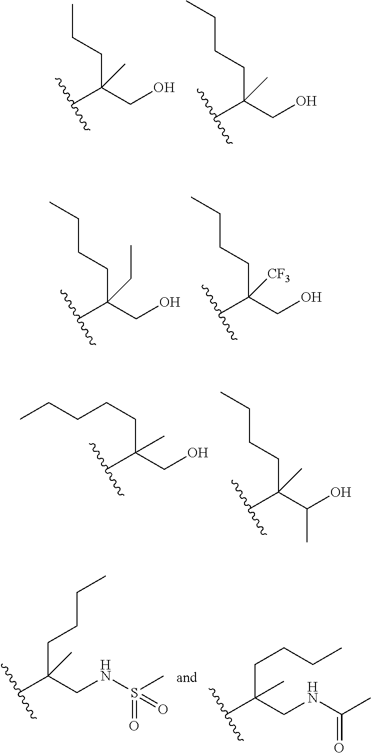

In certain embodiments of a compound of Formula (J) or (I), R⁴ is selected from the group consisting of:

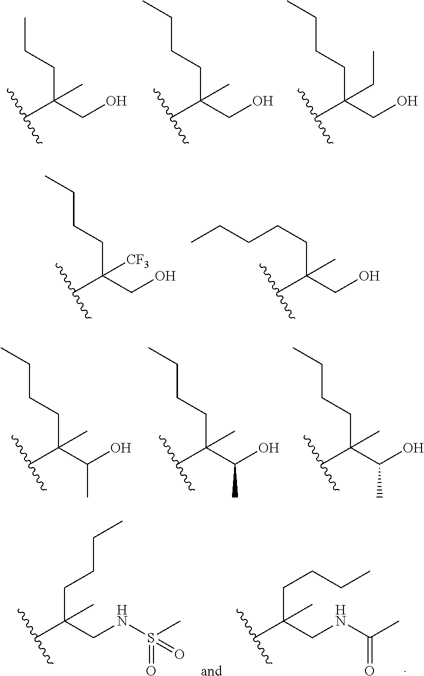

In certain embodiments of a compound of Formula (J) or (I), R⁴ is selected from the group consisting of:

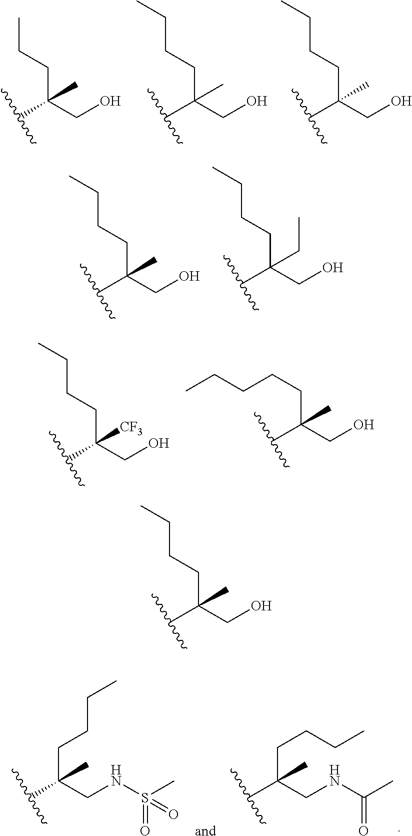

In certain embodiments of a compound of Formula (J) or (I), R⁴ is selected from the group consisting of:

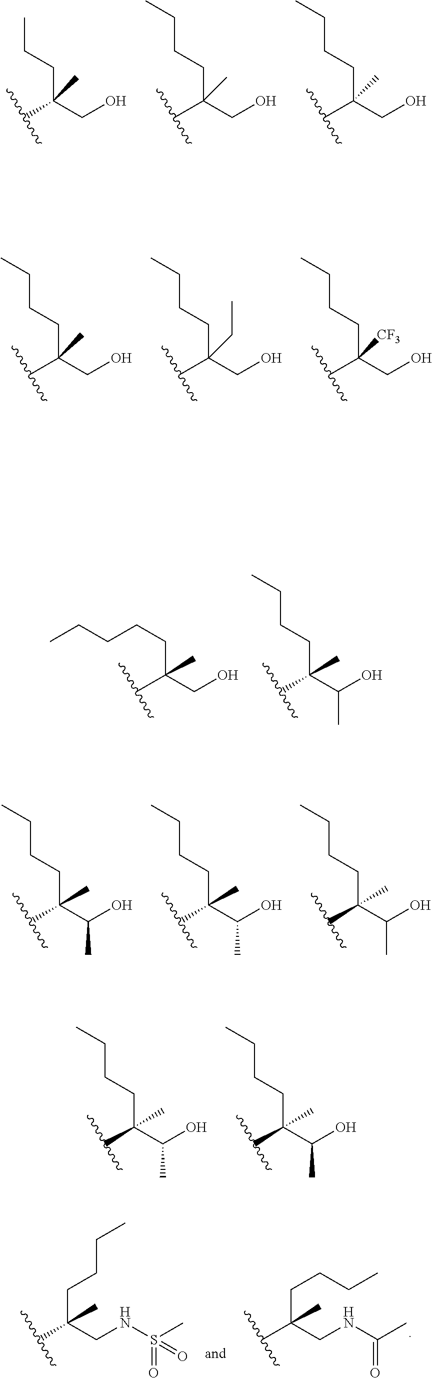

In certain embodiments of a compound of Formula (J) or (I), R⁴ is selected from the group consisting of:

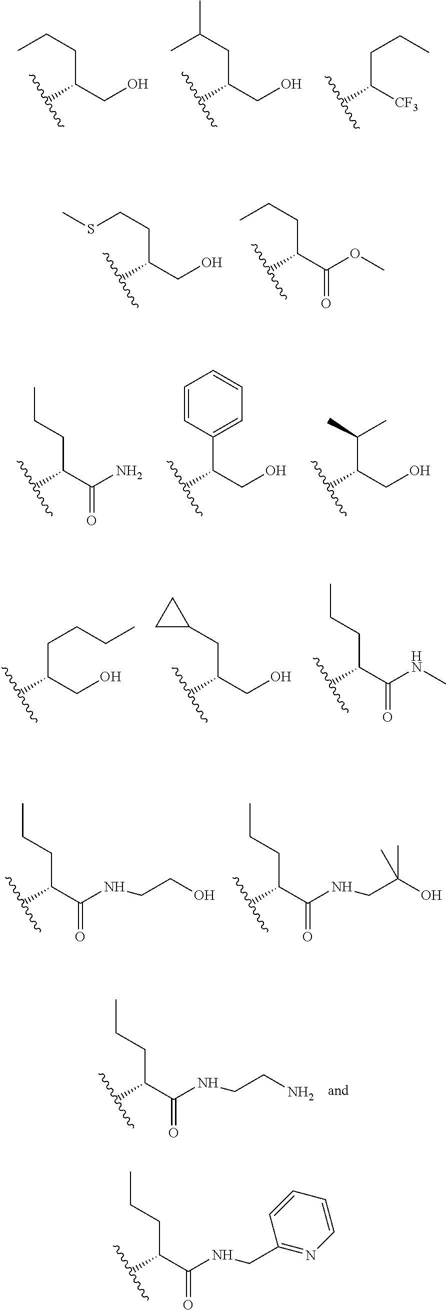

In certain embodiments of a compound of Formula (J) or (I), R⁴ is selected from the group consisting of:

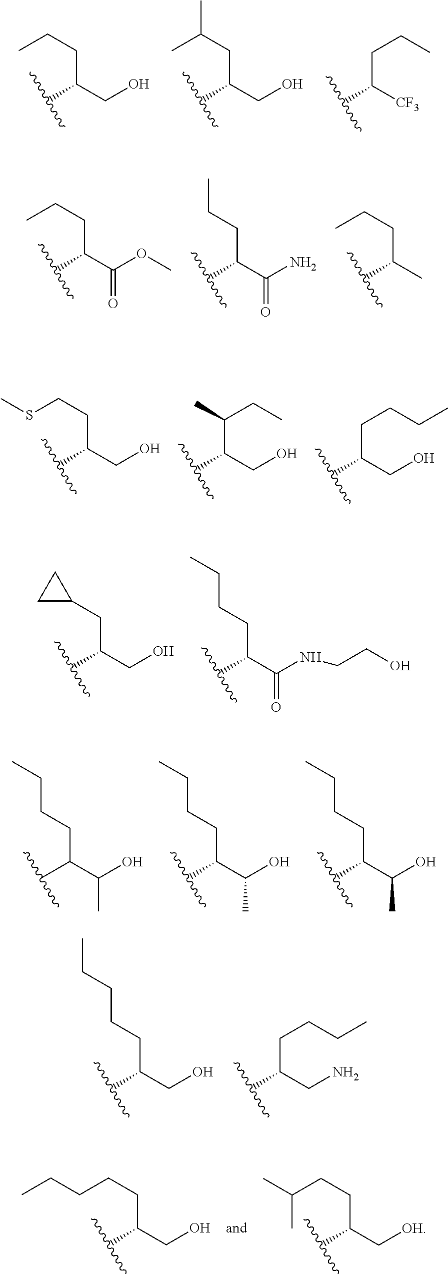

In certain embodiments of a compound of Formula (J) or (I), R⁴ is selected from the group consisting of:

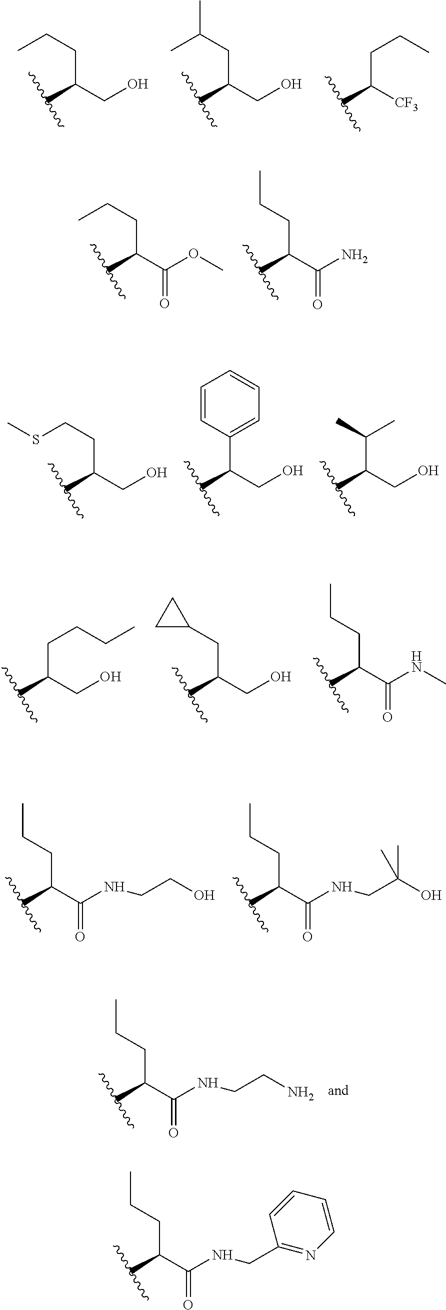

In certain embodiments of a compound of Formula (J) or (I), R⁴ is selected from the group consisting of

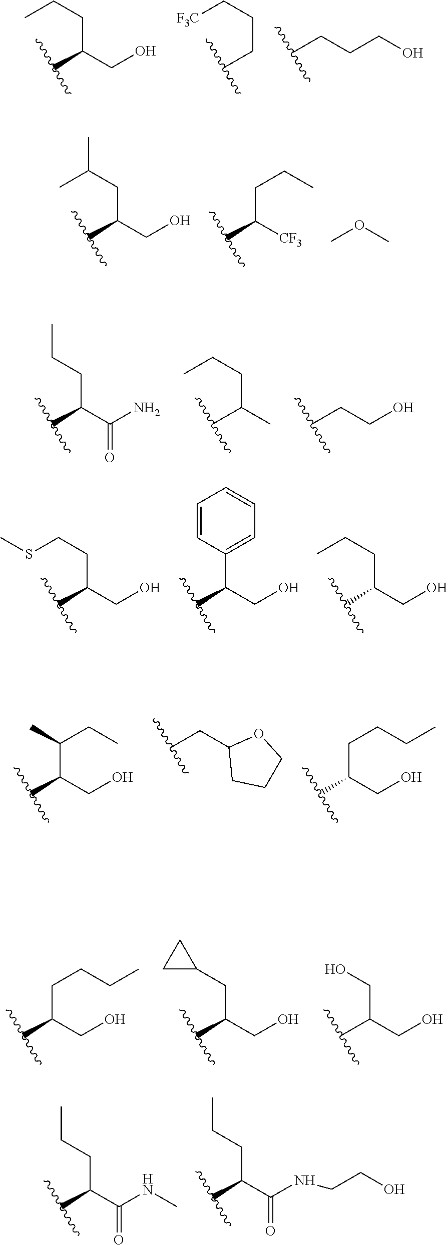

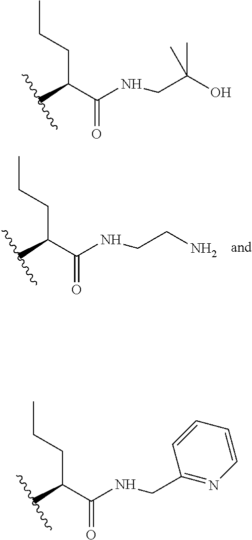 [SMILES: *[C@@H](CCC)C(=O)NCC(C)(C)O ; *[C@@H](CCC)C(=O)NCCN ; *[C@@H](CCC)C(=O)NCc1ccccn1]

In certain embodiments of a compound of Formula (J) or (I), R⁴ is

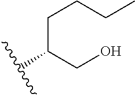 [SMILES: *[C@@H](CO)CCCC]

In certain embodiments of a compound of Formula (J) or (I), R⁴ is

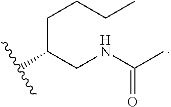 [SMILES: *[C@H](CCCC)CNC(C)=O]

In certain embodiments of a compound of Formula (J) or (I), R⁴ is

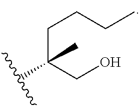

In certain embodiments of a compound of Formula (J) or (I), R⁴ is

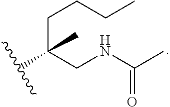

In certain embodiments of a compound of Formula (J) or (I), R⁴ is

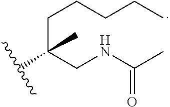

In certain embodiments, the compound of Formula (J) or (I) is a compound of Formula (II)

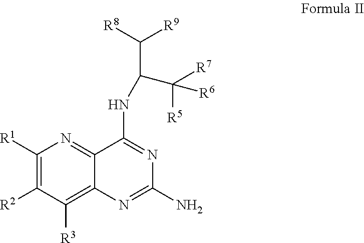

or a pharmaceutically acceptable salt thereof, wherein:
R⁵ is selected from the group consisting of hydrogen, halogen, and methyl; R⁶ is selected from the group consisting of hydrogen, halogen, and methyl; or R⁵ and R⁶ together form an oxo group;
R⁷ is selected from the group consisting of hydrogen, halogen, OR^{a} and NR^{a}R^{b}; R⁸ is selected from the group consisting of hydrogen and methyl;
R⁹ is is selected from the group consisting of C₁₋₄ alkyl, C₃₋₅cycloalkyl, and —S—C₁₋₄alkyl;
R^{a} and R^{b} are independently selected from the group consisting of hydrogen and C₁₋₆alkyl; wherein each C₁₋₆alkyl is optionally substituted with 1 to 3 substituents independently selected from the group consisting of halogen, hydroxyl, and pyridyl; and R¹, R², and R³ are as otherwise defined herein.

For example, in Formula (II), (IIa), and (IIb), R¹ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups; R² is selected from the group consisting of hydrogen, halogen, C₁₋₆alkyl, CN, —NR^{a}R^{b}, —S(O)₁₋₂R^{a} and OR^{a}, wherein C₁₋₆alkyl is optionally substituted with 1 to 5 R²⁰ groups; and R³ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups;

In certain embodiments, the compound of Formula (II) is a compound of Formula (IIa)

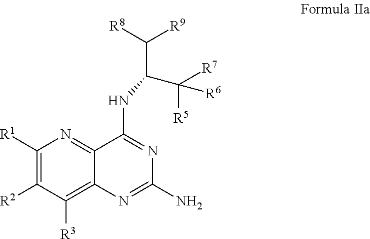

In certain embodiments, the compound of Formula (II) is a compound of Formula (IIb)

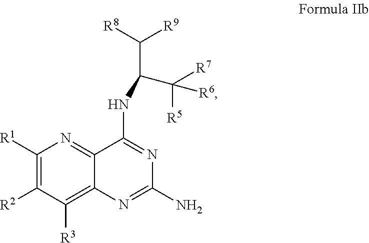

In certain embodiments of the compound of Formula (II), (IIa), or (IIb), R⁵ is hydrogen; R⁶ is hydrogen; or R⁵ and R⁶ together form an oxo group; R⁷ is OR^{a} or NR^{a}R^{b};

R⁸ is hydrogen; R⁹ is C₁₋₄ alkyl, cyclopropyl or —SCH₃; R^{a} and R^{b} are independently selected from the group consisting of hydrogen and C₁₋₄alkyl; wherein each C₁₋₄alkyl is optionally substituted with 1 to 3 substituents independently selected from halogen, hydroxyl, pyrid-2-yl, and CF₃, and R¹, R², and R³ are as otherwise defined herein. In certain embodiments, R^{a} and R^{b} are hydrogen. In certain embodiments, R⁷ is OH or NH₂. In certain embodiments, R¹ and R² are hydrogen.

In certain embodiments of a compound of Formula (IIa),

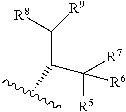

is selected from

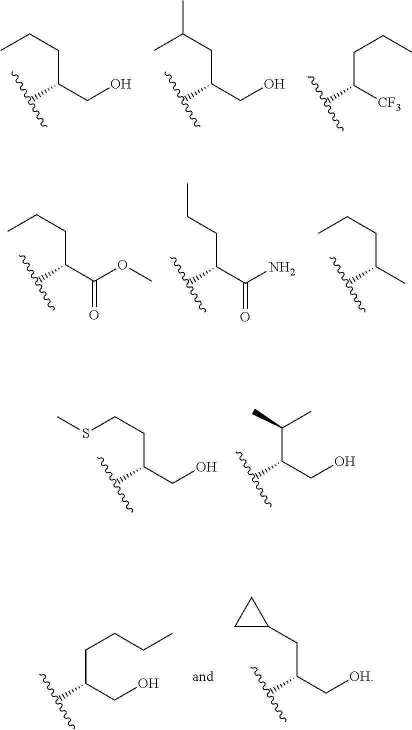

In certain embodiments of a compound of Formula (IIa),

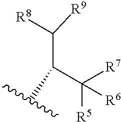

is selected from

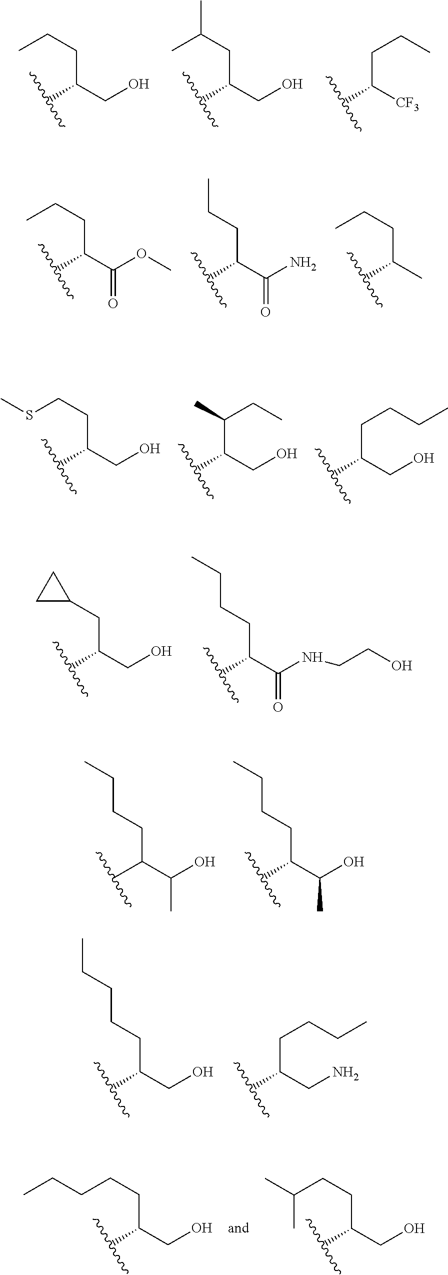

In certain embodiments of a compound of formula (lib),

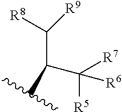

is selected from

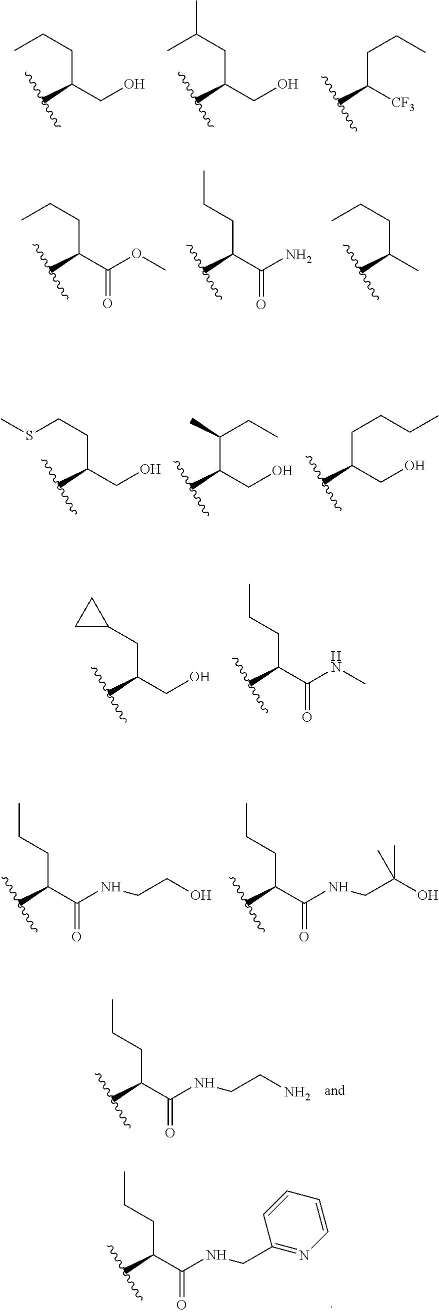

In certain embodiments of a compound of formula (lib),

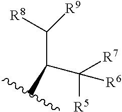

is selected from

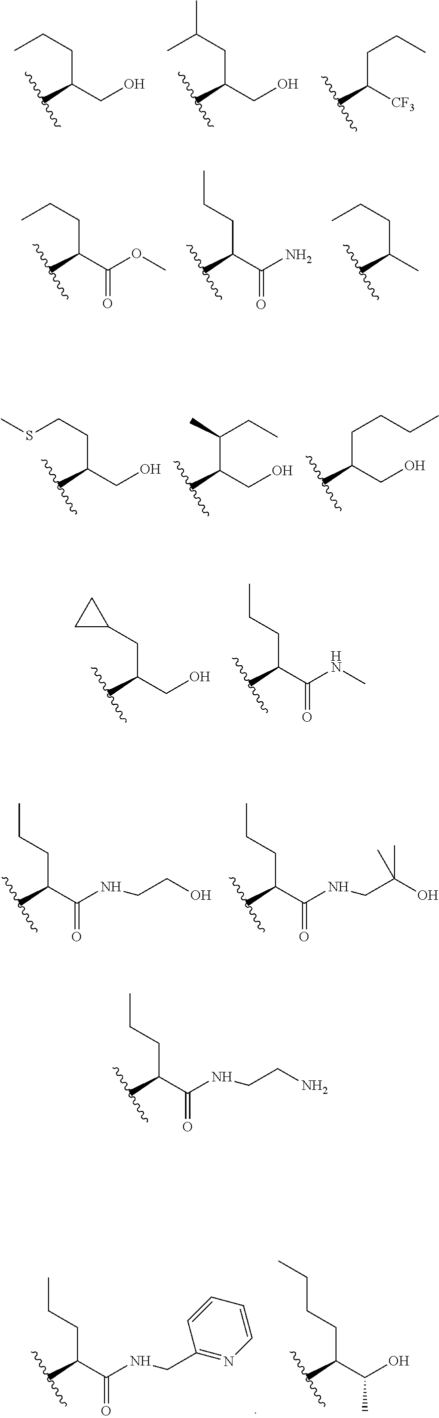

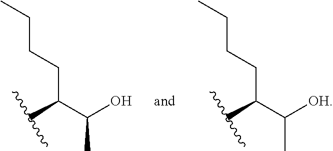

In certain embodiments of the compound of Formula (II), (IIa), or (IIb), R⁵ is hydrogen, R⁶ is hydrogen, or R⁵ and R⁶ together form an oxo group, R⁷ is OR^{a} or NR^{a}R^{b}, R⁸ is hydrogen, R⁹ is C₁₋₄alkyl, cyclopropyl or —SCH3, and R^{a} and R^{b} are independently selected from the group consisting of hydrogen and C₁₋₄alkyl; wherein each C₁₋₄alkyl is optionally substituted with 1 to 3 substituents independently selected from halogen, hydroxyl, pyrid-2-yl, and CF₃. In certain embodiments of the compound of Formula (II), (IIa), or (IIb), R⁷ is OH or NH₂.

In certain embodiments of a compound of Formula (J), Formula (I), or Formula (II), the compound is a compound of Formula (III)

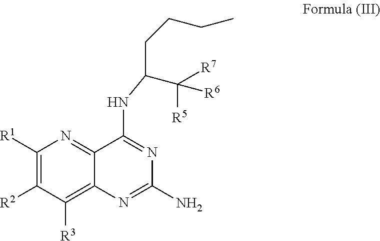

wherein
R⁵ is hydrogen;
R⁶ is hydrogen; or R⁵ and R⁶ together form an oxo group;
R⁷ is selected from the group consisting of OR and NR^{a}R^{b};
R^{a} and R^{b} are independently selected from the group consisting of hydrogen and C₁₋₃alkyl;
wherein each C₁₋₃alkyl is optionally substituted with 1 to 3 substituents independently selected from the group consisting of halogen and hydroxyl and R¹, R², and R³ are as otherwise defined herein.

In certain embodiments the compound of Formula (III) is a compound of Formula (IIIa)

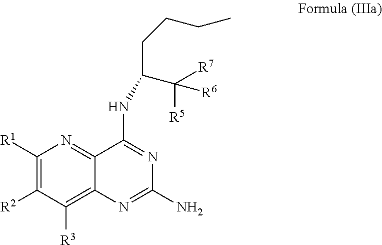

In certain embodiments the compound of Formula (III) is a compound of Formula (IIIb)

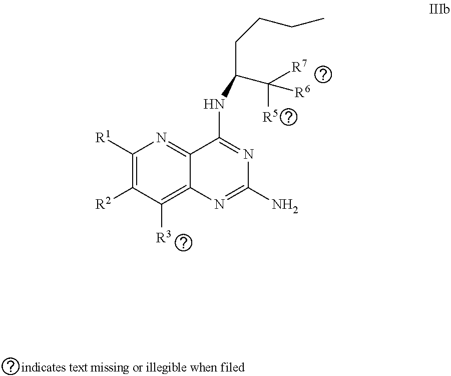

In certain embodiments of the compound of Formula (III), (IIIa), or (IIIb), R⁵ and R⁶ are both hydrogen and R⁷ is OR^{a}, wherein R^{a} is hydrogen or C₁₋₃alkyl. In certain embodiments of the compound of Formula (III), (IIIa), or (IIIb), R⁵ and R⁶ are both hydrogen and R⁷ is OH. In certain embodiments of the compound of Formula (III), (IIIa), or (IIIb), R¹, R², R⁵, and R⁶ are each hydrogen, and R⁷ is OH.

In certain embodiments of the compound of Formula (III), (IIIa), or (IIIb), R⁵ and R⁶ together form an oxo group and R⁷ is selected from the group consisting of OR^{a} and NR^{a}R^{b}, wherein R^{a} and R^{b} are independently selected from the group consisting of hydrogen and C₁₋₃alkyl. In certain embodiments of the compound of Formula (III), (IIIa), or (IIIb), R⁵ and R⁶ together form an oxo group and R⁷ is selected from the group consisting of OR^{a} and NR^{a}R^{b}, wherein R^{a} and R^{b} are independently selected from the group consisting of hydrogen and methyl.

In certain embodiments of a compound of Formula (J), or Formula (I), the compound is a compound of Formula (IV):

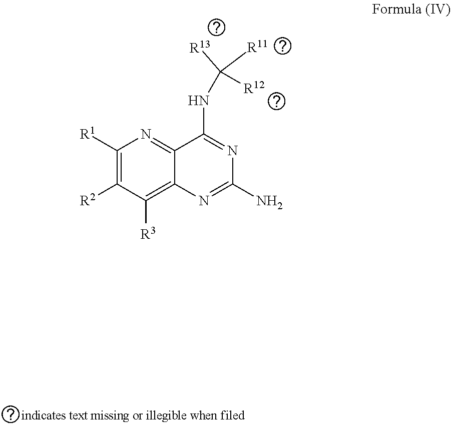

The R¹, R², and R³ groups of Formula (IV) are as defined above for Formula (J) or (I). The R¹¹, R¹² and R¹³ groups are as defined above for R⁴ in Formula (J) or Formula (I).

In certain embodiments, the compound of Formula (IV), or a pharmaceutically acceptable salt thereof, is a compound of Formula (IVa):

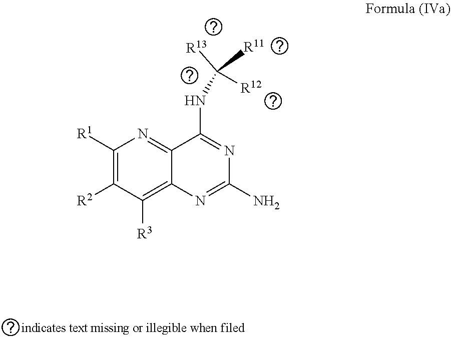

In certain embodiments, the compound of Formula (IV), or a pharmaceutically acceptable salt thereof, is a compound of Formula (IVb):

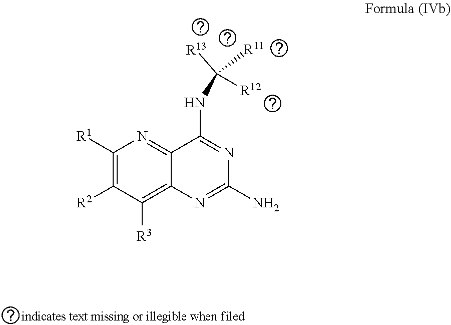

The groups R¹, R², R³, R¹¹, R¹² and R¹³ of Formula (IVa) and (IVb) are as defined for Formula (J), (I) or (IV) above, or as defined below, or any combination thereof.

R¹ of Formula (IV), (IVa) and (IVb) can be any suitable group selected from hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups. In certain embodiments, R¹ is selected from hydrogen, halogen, C₁₋₆ alkyl, CN, and OR^{a}, wherein C₁₋₆ alkyl is optionally substituted with 1 to 5 R²⁰ groups. In certain embodiments, R¹ can be hydrogen, halogen, and C₁₋₃ alkyl, wherein C₁₋₃ alkyl is optionally substituted with 1 to 5 halogen groups. In certain embodiments, R¹ can be hydrogen, fluoro, chloro, bromo, methyl or ethyl, wherein each methyl or ethyl group is optionally substituted with 1 to 5 halogen groups. In certain embodiments, R¹ can be hydrogen, fluoro, chloro, bromo, methyl or ethyl, wherein each methyl or ethyl group is optionally substituted with 1 to 5 fluoro groups. In certain embodiments, R¹ can be hydrogen, methyl, fluoro, chloro, and CF₃. In certain embodiments, R¹ can be hydrogen. In certain embodiments, R¹ is selected from hydrogen, halogen, NH₂, C₁₋₆ alkyl, CN, and OR^{a}, wherein C₁₋₆ alkyl is optionally substituted with 1 to 5 R²⁰ groups.

R² of Formula (IV), (IVa) and (IVb) can be any suitable group selected from hydrogen, halogen, C₁₋₄alkyl, CN, —NR^{a}R^{b}, —S(O)₁₋₂R^{a} and OR^{a}, wherein C₁₋₄alkyl is optionally substituted with 1 to 5 R²⁰ groups. In certain embodiments, R² is selected from hydrogen, halogen, C₁₋₆ alkyl, CN, and OR^{a}, wherein C₁₋₆ alkyl optionally substituted with 1 to 5 R²⁰ groups. In certain embodiments, R² is selected from hydrogen, halogen, C₁₋₃ alkyl, CN and OR^{a}, wherein C₁₋₃ alkyl is optionally substituted with 1 to 5 halogen groups. In certain embodiments, R² is selected from hydrogen, methyl, ethyl, fluoro, chloro, bromo, CF₃, CN, OH, OMe, and OEt. In certain embodiments, R² is selected from hydrogen, methyl, fluoro, and chloro. In certain embodiments, R² is selected from hydrogen and fluoro. In certain embodiments, R² is selected from hydrogen, halogen, NH₂, C₁₋₆ alkyl, CN, and OR^{a}, wherein C₁₋₆ alkyl is optionally substituted with 1 to 5 R²⁰ groups. In certain embodiments, R² is selected from hydrogen, methyl, ethyl, NH₂, fluoro, chloro, bromo, CF₃, CN, OH, OMe, and OEt.

R³ of Formula (IV), (IVa) and (IVb) can be any suitable group selected from hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups. In certain embodiments, R³ is selected from hydrogen, halogen, C₁₋₆ alkyl, CN, and OR^{a}, wherein C₁₋₆ alkyl is optionally substituted with 1 to 5 R²⁰ groups. In certain embodiments, R³ can be selected from hydrogen, halogen, and C₁₋₃ alkyl. In certain embodiments, R³ can be selected from hydrogen, methyl, fluoro, and chloro. In certain embodiments, R³ can be selected from hydrogen and methyl. In certain embodiments, R³ is selected from hydrogen, halogen, NH₂, C₁₋₆ alkyl, CN, and OR^{a}, wherein C₁₋₆ alkyl is optionally substituted with 1 to 5 R²⁰ groups.

In certain embodiments, the compound of Formula (IV), (IVa) or (IVb), or a pharmaceutically acceptable salt thereof, is the compound wherein R¹ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups, R² is selected from the group consisting of hydrogen, halogen, C₁₋₆alkyl, CN, —NR^{a}R^{b}, —S(O)₁₋₂R^{a} and OR^{a}, wherein C₁₋₄alkyl is optionally substituted with 1 to 5 R²⁰ groups, and R³ is selected from the group consisting of hydrogen, halogen, C₁₋₆alkyl, CN, —NR^{a}R^{b}, —S(O)₁₋₂R^{a}, and OR^{a}, wherein C₁₋₄alkyl is optionally substituted with 1 to 5 R²⁰ groups.

In certain embodiments, the compound of Formula (IV), (IVa) or (IVb), or a pharmaceutically acceptable salt thereof, is the compound wherein R¹ is selected from the group consisting of hydrogen, halogen, and C₁₋₃ alkyl, wherein C₁₋₃ alkyl is optionally substituted with 1 to 5 halogen groups, R² is selected from the group consisting of hydrogen, halogen, C₁₋₃ alkyl, CN and OR^{a}, wherein C₁₋₃ alkyl is optionally substituted with 1 to 5 halogen groups, and R³ is selected from the group consisting of hydrogen, halogen, and C₁₋₃ alkyl.

In certain embodiments, the compound of Formula (IV), (IVa) or (IVb), or a pharmaceutically acceptable salt thereof, is the compound wherein R¹ is selected from the group consisting of hydrogen, methyl, fluoro, chloro, and CF₃, R² is selected from the group consisting of hydrogen, methyl, ethyl, fluoro, chloro, bromo, CF₃, CN, OH, OMe, and OEt, and R³ is selected from the group consisting of hydrogen, methyl, fluoro, and chloro.

In certain embodiments, the compound of Formula (IV), (IVa) or (IVb), or a pharmaceutically acceptable salt thereof, is the compound wherein R¹ is selected from the group consisting of hydrogen, methyl, fluoro, chloro, and CF₃, R² is selected from the group consisting of hydrogen, methyl, ethyl, NH₂, fluoro, chloro, bromo, CF₃, CN, OH, OMe, and OEt, and R³ is selected from the group consisting of hydrogen, methyl, fluoro, and chloro.

In certain embodiments, the compound of Formula (IV), (IVa) or (IVb), or a pharmaceutically acceptable salt thereof, is the compound wherein R¹ is hydrogen, R² is selected from the group consisting of hydrogen, methyl, ethyl, fluoro, chloro, and bromo, and R³ is selected from the group consisting of hydrogen and methyl.

In certain embodiments, the compound of Formula (IV), (IVa) or (IVb), or a pharmaceutically acceptable salt thereof, is the compound wherein R¹ is hydrogen. R² is selected from the group consisting of hydrogen and fluoro, and R³ is selected from the group consisting of hydrogen and methyl.

In certain embodiments, R¹¹ of Formula (IV), (IVa) and (IVb) can be any suitable group selected from hydrogen, C₁₋₂ alkyl, C₃₋₆ cycloalkyl, and C₁₋₃haloalkyl. In certain embodiments, the compound of Formula (IV), (IVa) or (IVb), or a pharmaceutically acceptable salt thereof, is the compound wherein R¹¹ is selected from the group consisting of hydrogen, C₁₋₂ alkyl and C₁₋₂ haloalkyl. In certain embodiments, the compound of Formula (IV), (IVa) or (b), or a pharmaceutically acceptable salt thereof, is the compound wherein R¹¹ is selected from the group consisting of C₁₋₂ alkyl and C₁₋₂ haloalkyl. In certain embodiments, the compound of Formula (IV), (IVa) or (IVb), or a pharmaceutically acceptable salt thereof, is the compound wherein R¹¹ can be selected from hydrogen, methyl, ethyl or CF₃. In certain embodiments, the compound of Formula (IV), (IVa) or (IVb), or a pharmaceutically acceptable salt thereof, is the compound wherein R¹¹ can be selected from methyl, ethyl or CF₃. In certain embodiments, the compound of Formula (IV), (IVa) or (b), or a pharmaceutically acceptable salt thereof, is the compound wherein R¹¹ can be selected from hydrogen, methyl, or CF₃. In certain embodiments, the compound of Formula (IV), (IVa) or (IVb), or a pharmaceutically acceptable salt thereof, is the compound wherein R¹¹ can be selected from methyl, or CF₃. In certain embodiments, the compound of Formula (IV), (IVa) or (IVb), or a pharmaceutically acceptable salt thereof, is the compound wherein R¹¹ can be selected from hydrogen or methyl. In certain embodiments, the compound of Formula (IV), (IVa) or (IVb), or a pharmaceutically acceptable salt thereof, wherein R¹ is selected from the group consisting of methyl and CF₃. In certain embodiments, the compound of Formula (IV), (IVa) or (IVb), or a pharmaceutically acceptable salt thereof, is the compound wherein R¹¹ is methyl. In certain embodiments, the compound of Formula (IV), (IVa) or (b), or a pharmaceutically acceptable salt thereof, is the compound wherein R¹¹ is hydrogen.

R¹² of Formula (IV), (IVa) and (IVb) can be any suitable group selected from C₁₋₃ alkyl, halogen, —OR^{a}, —NR^{a}R^{b}, CN, —C(O)R^{a}, —(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₃ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur, wherein the C₁₋₃ alkyl group is optionally substituted with 1 to 5 substituents independently selected from halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₃ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur.

In certain embodiments, the compound of Formula (IV), (IVa) or (IVb), or a pharmaceutically acceptable salt thereof, wherein R¹² can be selected from C₁₋₂alkyl, —C(O)NR^{a}R^{b}, and 5 membered heteroaryl having 1 to 3 nitrogen heteroatoms, wherein C₁₋₂ alkyl is optionally substituted with 1 to 5 substituents independently selected from halogen, —OH, —NR^{a}R^{b}, —NR^{a}C(O)R^{b}, —NR^{a}S(O)₂R^{b}, and C₁₋₃ haloalkyl, and each R^{a} and R^{b} is independently selected from the group consisting of hydrogen and C₁₋₃ alkyl, wherein each C₁₋₃ alkyl is optionally substituted with 1 to 3 substituents independently selected from hydroxyl and amino. In certain embodiments, the compound of Formula (IV), (IVa) or (IVb), or a pharmaceutically acceptable salt thereof, wherein R¹² is C₁₋₂ alkyl, optionally substituted with 1 to 3 substituents independently selected from halogen, —OH, —NH₂, —NHC(O)—C₁₋₃ alkyl, —NHS(O)₂—C₁₋₃ alkyl, and C₁₋₃ haloalkyl. In certain embodiments, the compound of Formula (IV), (IVa) or (IVb), or a pharmaceutically acceptable salt thereof, wherein R¹² is methyl or ethyl, each optionally substituted with 1 or 2 substituents independently selected from halogen, —OH, —NH₂, —NHC(O)—C₁₋₃ alkyl, and C₁₋₃ haloalkyl. In certain embodiments, the compound of Formula (IV), (IVa) or (IVb), or a pharmaceutically acceptable salt thereof, wherein R¹² is methyl or ethyl, wherein the methyl or ethyl is substituted with 1 or 2 substituents independently selected from —OH and —NHC(O)CH₃. In certain embodiments, the compound of Formula (IV), (IVa) or (b), or a pharmaceutically acceptable salt thereof, wherein R¹² can be selected from CH₂OH, CH₂CH₂OH, CH(Me)OH, CH(CH₂F)OH, CH(CHF₂)OH, CH(CF₃)OH, CF₃, CH₂NH₂, CH₂NHC(O)Me, CH(CH₂F)NHC(O)Me, CH₂NHS(O)₂Me, C(O)NH₂, C(O)NHMe, C(O)NH—CH₂CH₂OH, C(O)NH—CH₂CH₂NH₂, C(O)NH-(pyridin-2-ylmethyl), imidazolyl, and triazolyl. In certain embodiments, the compound of Formula (IV), (IVa) or (IVb), or a pharmaceutically acceptable salt thereof, wherein R¹² can be selected from CH₂OH, CH(Me)OH, CH(CH₂F)OH, and CH₂NHC(O)Me. In certain embodiments, the compound of Formula (IV), (IVa) or (IVb), or a pharmaceutically acceptable salt thereof, wherein R¹² can be selected from CH₂OH, CH(Me)OH, and CH₂NHC(O)Me. In certain embodiments, the compound of Formula (IV), (IVa) or (IVb), or a pharmaceutically acceptable salt thereof, wherein R¹² is —CH₂OH or —CH₂NC(O)CH₃.

In certain embodiments, the compound of Formula (IV), (IVa) or (IVb), or a pharmaceutically acceptable salt thereof, wherein R¹² is C₁₋₂ alkyl substituted with —NR^{a}C(O)R^{b}, wherein each R^{a} and R^{b} is independently selected from the group consisting of hydrogen and C₁₋₃ alkyl, wherein each C₁₋₃ alkyl is optionally substituted with 1 to 3 substituents independently selected from hydroxyl and amino.

R¹³ of Formula (IV), (IVa) and (IVb) can be any suitable group selected from C₁₋₆ alkyl, halogen, —OR^{a}, —NR^{a}R^{b}, CN, —C(O)R^{a}, —C(O)OR^{a}, —C(O)NR⁸R, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur, wherein the C₁₋₆ alkyl is optionally substituted with 1 to 5 substituents independently selected from halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR, —NR^{a}C(O)OR^{b}, —SR^{a}, —S(O)₁₋₂R^{a}, —S(O)₂NR^{a}R^{b}, —NR^{a}S(O)₂R^{b}, C₁₋₆ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur.

In certain embodiments, the compound of Formula (IV), (IVa) or (IVb), or a pharmaceutically acceptable salt thereof, is the compound wherein R¹³ is C₃₋₆alkyl optionally substituted with 1 to 2 substituents independently selected from halogen and —OH. In certain embodiments, the compound of Formula (IV), (IVa) or (IVb), or a pharmaceutically acceptable salt thereof, is the compound wherein R¹³ is C₃₋₆ alkyl optionally substituted with 1 to 2 halogen substituents. In certain embodiments, the compound of Formula (IV), (IVa) or (IVb), or a pharmaceutically acceptable salt thereof, is the compound wherein R¹³ is C₃₋₆ alkyl. Representative C₃₋₆ alkyl groups for R¹³ include, but are not limited to, n-propyl, iso-propyl, n-butyl, sec-butyl, iso-butyl, tert-butyl, n-pentyl, tert-pentyl, neopentyl, isopentyl, sec-pentyl and 3-pentyl. In certain embodiments, the compound of Formula (IV), (IVa) or (IVb), or a pharmaceutically acceptable salt thereof, is the compound wherein R¹³ is propyl, butyl or pentyl. In certain embodiments, the compound of Formula (IV), (IVa) or (IVb), or a pharmaceutically acceptable salt thereof, is the compound wherein R¹³ is n-propyl, n-butyl or n-pentyl. In certain embodiments, the compound of Formula (IV), (IVa) or (IVb), or a pharmaceutically acceptable salt thereof, is the compound wherein R¹³ is propyl or butyl.

R²⁰ of Formula (IV), (IVa) and (IVb) can be any suitable group selected from halogen, C₁₋₆haloalkyl, CN, —NR^{a}R^{b}, S(O)₁₋₂R^{a}, and OR^{a}. In certain embodiments, each R²⁰ can independently be selected from halogen, CN, —NR^{a}R^{b}, and OR^{a}. In certain embodiments, each R²⁰ can independently be selected from halogen, CN, —NR^{a}R^{b}, and OR^{a}. In certain embodiments, each R²⁰ can independently be halogen. In certain embodiments, each R²⁰ can independently be selected from fluoro, chloro, bromo, CN, —NH₂, OH, OMe, and OEt. In certain embodiments, each R²⁰ can independently be selected from fluoro and chloro.

R^{a} and R^{b} of Formula (IV), (IVa) and (IVb) can each independently be any suitable group selected from the group consisting of hydrogen and C₁₋₆alkyl;

wherein each C₁₋₆alkyl is optionally substituted with 1 to 5 substituents independently selected from halogen, hydroxyl, amino, 5 to 10 membered heteroaryl wherein the 5 to 10 membered heteroaryl has 1 to 3 heteroatoms selected from oxygen, nitrogen, and sulfur, and C₁₋₆haloalkyl. In certain embodiments, R^{a} and R^{b} can each independently be selected from hydrogen and C₁₋₃ alkyl, wherein each C₁₋₃ alkyl is optionally substituted with 1 to 3 substituents independently selected from halogen, hydroxyl, amino, and C₁₋₆ haloalkyl. In certain embodiments, R^{a} and R^{b} can each independently be selected from hydrogen and C₁₋₃ alkyl, wherein each C₁₋₃ alkyl is optionally substituted with 1 to 3 substituents independently selected from hydroxyl and amino. In certain embodiments, R^{a} and R^{b} can each independently be selected from hydrogen and C₁₋₃ alkyl, wherein each C₁₋₃ alkyl is optionally substituted with 1 substituent selected from hydroxyl and amino. In certain embodiments, R^{a} and R^{b} can each independently be selected from hydrogen and C₁₋₃ alkyl. In certain embodiments, R^{a} and R^{b} can each independently be selected from hydrogen, methyl, ethyl, propyl, butyl, CF₃, CH₂CF₃, CH₂CH₂CF₃, CH₂OH, CH₂CH₂OH, CH₂NH₂, and CH₂CH₂NH₂. In certain embodiments, R^{a} and R^{b} can each independently be selected from hydrogen, methyl, ethyl, CF₃, CH₂OH, CH₂CH₂OH, CH₂NH₂, and CH₂CH₂NH₂. In certain embodiments, R^{a} and R^{b} can each independently be selected from hydrogen, methyl, ethyl, CH₂CH₂OH, and CH₂CH₂NH₂. In certain embodiments, R^{a} and R^{b} can each independently be selected from hydrogen, methyl and ethyl. In certain embodiments, R^{a} and R^{b} can each independently be selected from hydrogen and methyl.

In certain embodiments, the compound of Formula (IV), (IVa) or (IVb), or a pharmaceutically acceptable salt thereof, is the compound wherein:

R¹ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups;
R² is selected from the group consisting of hydrogen, halogen, C₁₋₆alkyl, CN, —NR^{a}R^{b}, —S(O)₁₋₂R^{a} and OR^{a}, wherein C₁₋₆alkyl is optionally substituted with 1 to 5 R²⁰ groups;
R³ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups;
R¹¹ is selected from the group consisting of hydrogen, C₁₋₂ alkyl, C₃₋₆ cycloalkyl, and C₁₋₃ haloalkyl;
R¹² is selected from C₁₋₃ alkyl, halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₃ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur, wherein the C₁₋₃ alkyl group is optionally substituted with 1 to 5 substituents independently selected from halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₃ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur;
R¹³ is selected from C₁₋₆ alkyl, halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur, wherein the C₁₋₆ alkyl is optionally substituted with 1 to 5 substituents independently selected from halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur; each R²⁰ is independently selected from the group consisting of halogen, CN, —NR^{a}R^{b}, and OR^{a}; and
each R^{a} and R^{b} is independently selected from the group consisting of hydrogen and C₁₋₃ alkyl, wherein each C₁₋₃ alkyl is optionally substituted with 1 to 3 substituents independently selected from halogen, hydroxyl, amino, and C₁₋₆ haloalkyl.

In certain embodiments, the compound of Formula (IV), (IVa) or (IVb), or a pharmaceutically acceptable salt thereof, is the compound wherein:

R¹ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups;
R² is selected from the group consisting of hydrogen, halogen, C₁₋₆alkyl, CN, —NR^{a}R^{b}, —S(O)₁₋₂R^{a} and OR^{a}, wherein C₁₋₆alkyl is optionally substituted with 1 to 5 R²⁰ groups;
R³ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups;
R¹¹ is selected from the group consisting of C₁₋₂ alkyl, C₃₋₆ cycloalkyl, and C₁₋₃ haloalkyl;
R¹² is selected from C₁₋₃ alkyl, halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₃ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur, wherein the C₁₋₃ alkyl group is optionally substituted with 1 to 5 substituents independently selected from halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₃ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur;
R¹³ is selected from C₁₋₆ alkyl, halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur, wherein the C₁₋₆ alkyl is optionally substituted with 1 to 5 substituents independently selected from halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur; each R²⁰ is independently selected from the group consisting of halogen, CN, —NR^{a}R^{b}, and OR^{a}; and
each R^{a} and R^{b} is independently selected from the group consisting of hydrogen and C₁₋₃ alkyl, wherein each C₁₋₃ alkyl is optionally substituted with 1 to 3 substituents independently selected from halogen, hydroxyl, amino, and C₁₋₆ haloalkyl.

In certain embodiments, the compound of Formula (IV), (IVa) or (IVb), or a pharmaceutically acceptable salt thereof, is the compound wherein:

R¹ is selected from the group consisting of hydrogen, halogen, C₁₋₆ alkyl, CN, and OR^{a}, wherein C₁₋₆ alkyl is optionally substituted with 1 to 5 R²⁰ groups;
R² is selected from the group consisting of hydrogen, halogen, C₁₋₆ alkyl, CN, and OR^{a}, wherein C₁₋₆ alkyl optionally substituted with 1 to 5 R²⁰ groups; R³ is selected from the group consisting of hydrogen, halogen, C₁₋₆ alkyl, CN, and OR^{a}, wherein C₁₋₆ alkyl is optionally substituted with 1 to 5 R²⁰ groups;
R¹¹ is selected from the group consisting of hydrogen, C₁₋₂ alkyl, C₃₋₆ cycloalkyl, and C₁₋₃ haloalkyl;
R¹² is selected from C₁₋₃ alkyl, halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₃ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur, wherein the C₁₋₃ alkyl group is optionally substituted with 1 to 5 substituents independently selected from halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₃ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur;
R¹³ is selected from C₁₋₆ alkyl, halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur, wherein the C₁₋₆ alkyl is optionally substituted with 1 to 5 substituents independently selected from halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur; each R²⁰ is independently selected from the group consisting of halogen, CN, —NR^{a}R^{b}, and OR^{a}; and
each R^{a} and R^{b} is independently selected from the group consisting of hydrogen and C₁₋₃ alkyl, wherein each C₁₋₃ alkyl is optionally substituted with 1 to 3 substituents independently selected from halogen, hydroxyl, amino, and C₁₋₆ haloalkyl.

In certain embodiments, the compound of Formula (IV), (IVa) or (IVb), or a pharmaceutically acceptable salt thereof, is the compound wherein:

R¹ is selected from the group consisting of hydrogen, halogen, C₁₋₆ alkyl, CN, and OR^{a}, wherein C₁₋₆ alkyl is optionally substituted with 1 to 5 R²⁰ groups;
R² is selected from the group consisting of hydrogen, halogen, C₁₋₆ alkyl, CN, and OR^{a}, wherein C₁₋₆ alkyl optionally substituted with 1 to 5 R²⁰ groups;
R³ is selected from the group consisting of hydrogen, halogen, C₁₋₆ alkyl, CN, and OR^{a}, wherein C₁₋₆ alkyl is optionally substituted with 1 to 5 R²⁰ groups;
R¹¹ is selected from the group consisting of C₁₋₂ alkyl, C₃₋₆ cycloalkyl, and C₁₋₃ haloalkyl;
R¹² is selected from C₁₋₃ alkyl, halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₃ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur, wherein the C₁₋₃ alkyl group is optionally substituted with 1 to 5 substituents independently selected from halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₃ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur;
R¹³ is selected from C₁₋₆ alkyl, halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur, wherein the C₁₋₆ alkyl is optionally substituted with 1 to 5 substituents independently selected from halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur;
each R²⁰ is independently selected from the group consisting of halogen, CN, —NR^{a}R^{b}, and OR^{a}; and
each R^{a} and R^{b} is independently selected from the group consisting of hydrogen and C₁₋₃ alkyl, wherein each C₁₋₃ alkyl is optionally substituted with 1 to 3 substituents independently selected from halogen, hydroxyl, amino, and C₁₋₆ haloalkyl.

In certain embodiments, the compound of Formula (IV), (IVa) or (IVb), or a pharmaceutically acceptable salt thereof, wherein R¹¹ is methyl or CF₃, R¹² is —CH₂OH, —CH(Me)OH or —CH₂NHC(O)CH₃, and R¹³ is selected from the group consisting of propyl, butyl and pentyl.

In certain embodiments, the compound of Formula (IV), (IVa) or (Vb), or a pharmaceutically acceptable salt thereof, wherein R¹¹ is methyl or CF₃, R¹² is —CH₂OH, —CH(Me)OH, CH₂NHCH(CH₃)(CF₃) or —CH₂NHC(O)CH₃, and R¹³ is selected from the group consisting of propyl, butyl and pentyl.

In certain embodiments, the compound of Formula (IV), (IVa) or (IVb), or a pharmaceutically acceptable salt thereof, wherein R¹¹ is methyl, R¹² is —CH₂OH or —CH₂NHC(O)CH₃, and R¹³ is selected from the group consisting of propyl and butyl.

In certain embodiments, the compound of Formula (IV), or a pharmaceutically acceptable salt thereof, wherein the moiety

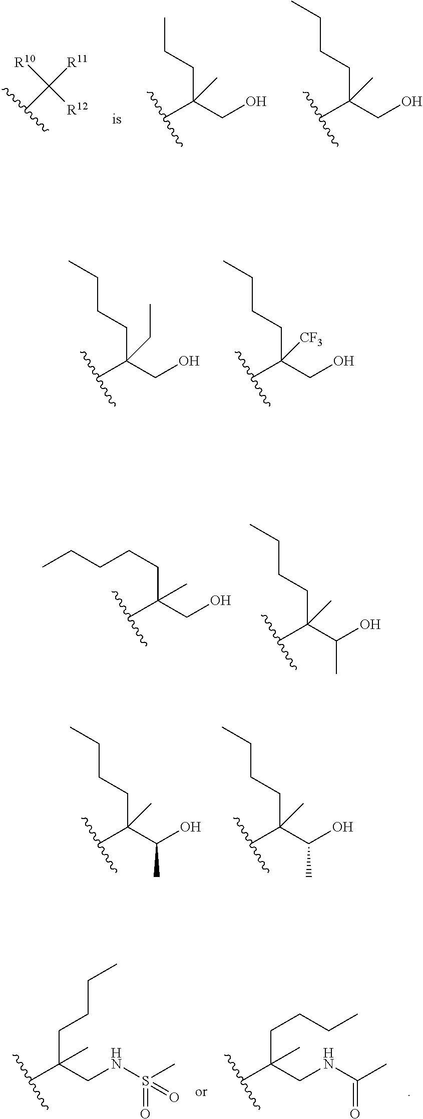

In certain embodiments, the compound of Formula (IV), or a pharmaceutically acceptable salt thereof, wherein the moiety

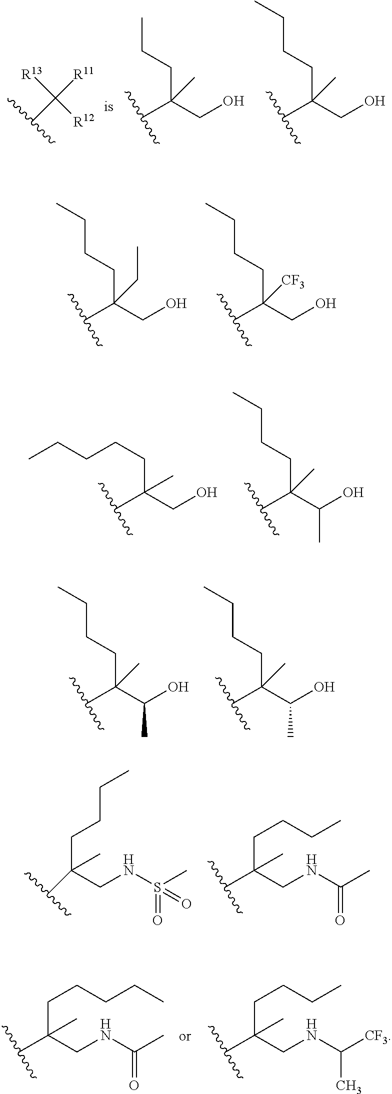

In certain embodiments, the compound of Formula (IV) or (IVa), or a pharmaceutically acceptable salt thereof, wherein the moiety

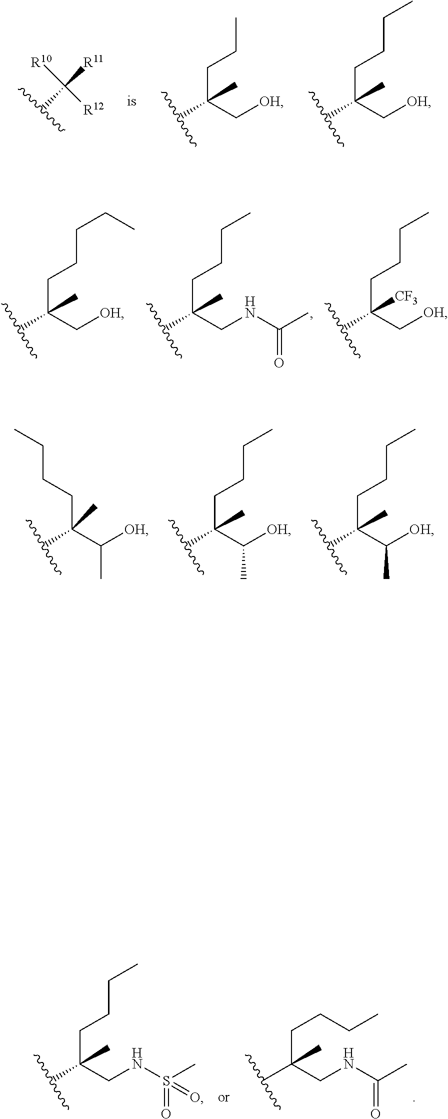

In certain embodiments, the compound of Formula (IV) or (IVa), or a pharmaceutically acceptable salt thereof, wherein the moiety

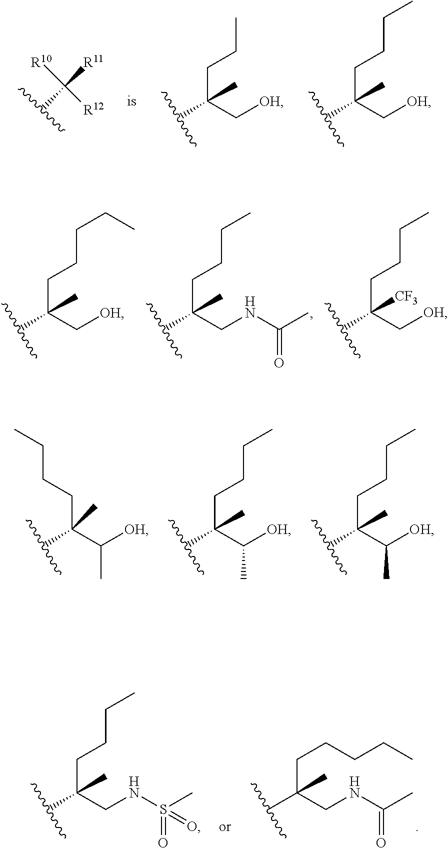

In certain embodiments, the compound of Formula (IV) or (IVa), or a pharmaceutically acceptable salt thereof, wherein the moiety

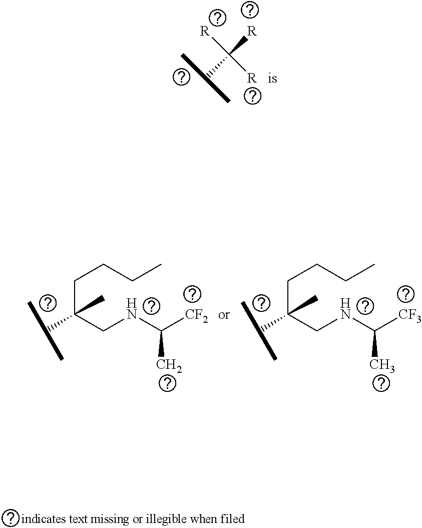

In certain embodiments, the compound of Formula (IV) or (IVa), or a pharmaceutically acceptable salt thereof, wherein the moiety

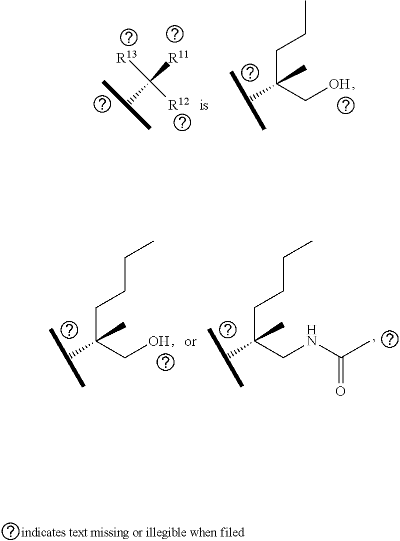

In certain embodiments, the compound of Formula (IV) or (IVa), or a pharmaceutically acceptable salt thereof, wherein the moiety

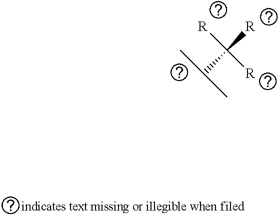

can also be drawn as the moiety

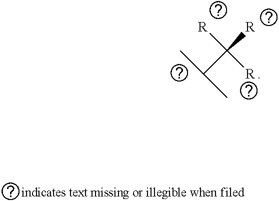

In certain embodiments, the compound of Formula (IV) or (IVb), or a pharmaceutically acceptable salt thereof, wherein the moiety

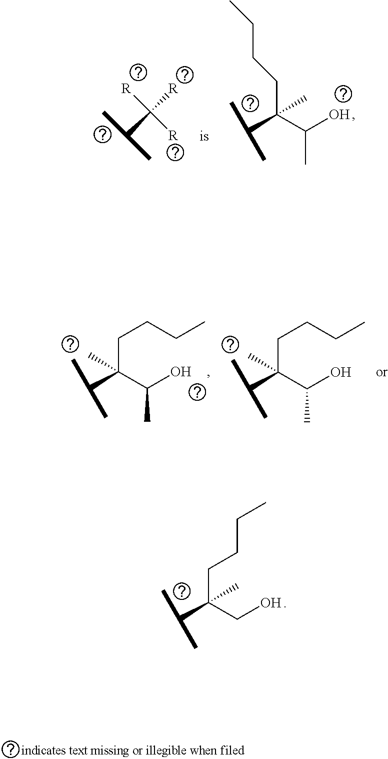

In certain embodiments, the compound of Formula (IV) or (IVb), or a pharmaceutically acceptable salt thereof, wherein the moiety

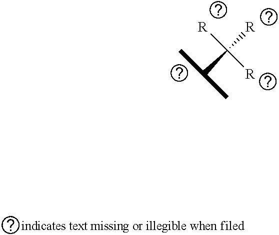

can also be drawn as the moiety

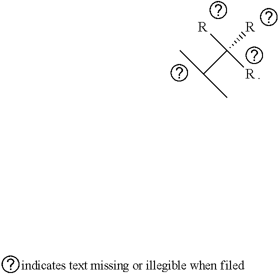

In certain embodiments, the compound of Formula (IV) or (IVa), or a pharmaceutically acceptable salt thereof, is a compound of Formula (IVc)

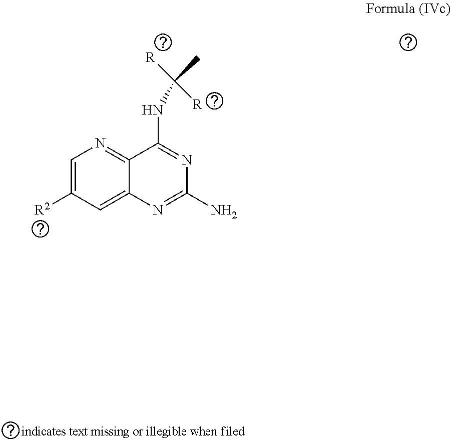

The R², R¹² and R¹³ groups of Formula (IVc) are as defined above for Formula (J), (I), (IV) or (IVa), or any combination thereof. For example, R² can be selected from hydrogen, halogen, C₁₋₃ alkyl, CN and OR^{a}, wherein C₁₋₃ alkyl is optionally substituted with 1 to 5 halogen groups, R¹² can be selected from C₁₋₂alkyl, —C(O)NR^{a}R^{b}, and 5 membered heteroaryl having 1 to 3 nitrogen heteroatoms, wherein C₁₋₂ alkyl is optionally substituted with 1 to 5 substituents independently selected from halogen, —OH, —NR^{a}R^{b}, —NR^{a}C(O)R^{b}, —NR^{a}S(O)₂R^{b}, and C₁₋₃ haloalkyl, and R¹³ can be C₃₋₆ alkyl optionally substituted with 1 to 2 substituents independently selected from halogen and —OH. In certain embodiments, the compound of Formula (IV), (IVa), or (IVc), or a pharmaceutically acceptable salt thereof, is a compound wherein R² can be selected from hydrogen, methyl, ethyl, fluoro, chloro, bromo, CF₃, CN, OH, OMe, and OEt, and R¹² can be selected CH₂OH, CH₂CH₂OH, CH(Me)OH, CH(CH₂F)OH, CH(CHF₂)OH, CH(CF₃)OH, CF₃, CH₂NH₂, CH₂NHC(O)Me, CH(CH₂F)NHC(O)Me, CH₂NHS(O)₂Me, C(O)NH₂, C(O)NHMe, C(O)NH—CH₂CH₂OH, C(O)NH—CH₂CH₂NH₂, C(O)NH-(pyridin-2-ylmethyl), imidazolyl, and triazolyl, and R¹³ can be propyl, butyl or pentyl. In certain embodiments, the compound of Formula (IV), (IVa), or (IVc), or a pharmaceutically acceptable salt thereof, is a compound wherein R² can be selected from hydrogen, methyl, fluoro, and chloro, and R¹² can be selected CH₂OH, CH(Me)OH, CH(CH₂F)OH, and CH₂NHC(O)Me, and R¹³ can be propyl, butyl or pentyl. In certain embodiments, the compound of Formula (IV), (IVa), or (IVc), or a pharmaceutically acceptable salt thereof, is a compound wherein R² is hydrogen or fluoro, R¹² is —CH₂OH or —CH₂NHC(O)CH₃, and R¹³ is selected from propyl and butyl. In certain embodiments, the compound of Formula (IV), (IVa), or (IVc), or a pharmaceutically acceptable salt thereof, is a compound wherein R² is hydrogen, chloro, or fluoro, R¹² is —CH₂OH or —CH₂NHC(O)CH₃, and R¹³ is selected from butyl or pentyl.

In certain embodiments, the compound of Formula (IV) or (IVa), or a pharmaceutically acceptable salt thereof, is a compound of Formula (IVd)

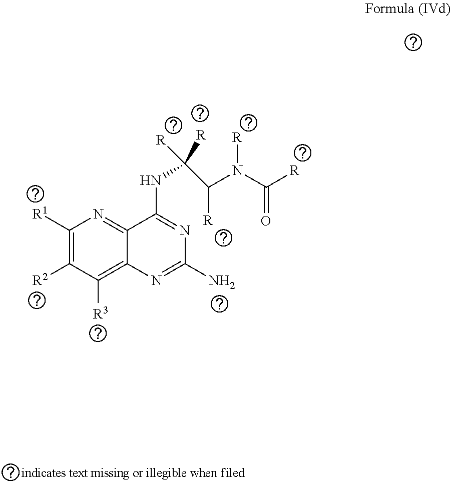

The R¹, R², R³, R¹¹, R¹³, R^{a} and R^{b} groups of Formula (IVd) can be as defined above for Formula (J), (I), (IV), or (IVa), or any combination thereof. R^{12a} can be any suitable group selected from hydrogen, C₁₋₂ alkyl and C₁₋₃ haloalkyl. In certain embodiments, the compound of Formula (IV), (IVa) or (IVd), or a pharmaceutically acceptable salt thereof, is a compound wherein R^{12a} can be selected from hydrogen, C₁₋₂ alkyl and C₁₋₃ haloalkyl. In certain embodiments, the compound of Formula (IV), (IVa) or (IVd), or a pharmaceutically acceptable salt thereof, is a compound wherein R^{12a} can be selected from hydrogen, methyl, ethyl and CF₃. In certain embodiments, the compound of Formula (IV), (IVa) or (IVd), or a pharmaceutically acceptable salt thereof, is a compound wherein R^{12a} can be hydrogen.

In certain embodiments, the compound of Formula (IVd), or a pharmaceutically acceptable salt thereof, is the compound wherein R¹ is selected from the group consisting of hydrogen, halogen, C₁₋₆ alkyl, CN, and OR^{a}, wherein C₁₋₆ alkyl is optionally substituted with 1 to 5 R²⁰ groups, R² is selected from the group consisting of hydrogen, halogen, C₁₋₆ alkyl, CN, and OR^{a}, wherein C₁₋₆ alkyl optionally substituted with 1 to 5 R²⁰ groups, R³ is selected from the group consisting of hydrogen, halogen, C₁₋₆ alkyl, CN, and OR^{a}, wherein C₁₋₆ alkyl is optionally substituted with 1 to 5 R²⁰ groups, R¹¹ is C₁₋₂ alkyl or CF₃, R^{12a} is selected from the group consisting of hydrogen, C₁₋₂ alkyl and C₁₋₃ haloalkyl, R¹³ is C₃₋₆ alkyl optionally substituted with 1 to 2 halogen substituents, each R²⁰ is independently selected from the group consisting of halogen, C₁₋₆haloalkyl, CN, —NR^{a}R^{b}, S(O)₁₋₂R^{a}, and OR^{a}, and each R^{a} and R^{b} is independently selected from the group consisting of hydrogen and C₁₋₃ alkyl, wherein each C₁₋₃ alkyl is optionally substituted with 1 to 3 substituents independently selected from halogen, hydroxyl, amino, and C₁₋₆ haloalkyl.

In certain embodiments, the compound of Formula (IVd), or a pharmaceutically acceptable salt thereof, is the compound wherein R¹ is selected from the group consisting of hydrogen, halogen, and C₁₋₃ alkyl, R² is selected from the group consisting of hydrogen, halogen, and C₁₋₃ alkyl, R³ is selected from the group consisting of hydrogen, halogen, and C₁₋₃ alkyl, R¹¹ is C₁₋₂ alkyl or CF₃, R^{12a} is selected from the group consisting of hydrogen, C₁₋₂ alkyl and C₁₋₃ haloalkyl, R¹³ is C₃₋₆ alkyl optionally substituted with 1 to 2 halogen substituents, and each R^{a} and R^{b} is independently selected from the group consisting of hydrogen and C₁₋₃alkyl, wherein each C₁₋₃ alkyl is optionally substituted with 1 to 3 substituents independently selected from halogen, hydroxyl, amino, and C₁₋₆ haloalkyl.

In certain embodiments, the compound of Formula (IVd), or a pharmaceutically acceptable salt thereof, has the structure:

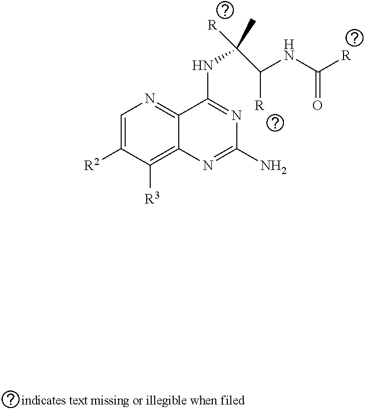

wherein R² is selected from the group consisting of hydrogen, methyl, fluoro, and chloro, R³ is selected from the group consisting of hydrogen and methyl, R^{12a} is selected from the group consisting of hydrogen, C₁₋₂ alkyl and C₁₋₃ haloalkyl, R¹³ is C₃₋₆ alkyl, and R^{b} is methyl or ethyl, each optionally substituted with hydroxyl or amino.

In certain embodiments, the compound of Formula (IVd), or a pharmaceutically acceptable salt thereof, has the structure:

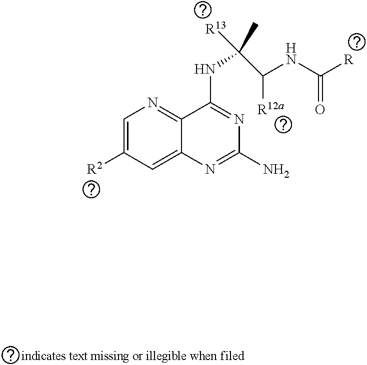

wherein R² is selected from the group consisting of hydrogen, methyl, fluoro, and chloro, R^{12a} is selected from the group consisting of hydrogen, C₁₋₂ alkyl and C₁₋₃ haloalkyl, R¹³ is C₃₋₆ alkyl, and R^{b} is methyl or ethyl, each optionally substituted with hydroxyl or amino. In certain embodiments, R² and R¹³ can be as defined above for Formula (J), (I), (IV), or (IVa), or any combination thereof.

In certain embodiments, the compound of Formula (IVd), or a pharmaceutically acceptable salt thereof, has the structure:

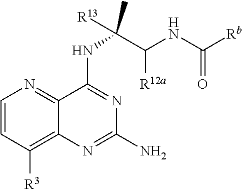

wherein R³ is selected from the group consisting of hydrogen and methyl, R^{12a} is selected from the group consisting of hydrogen, C₁₋₂ alkyl and C₁₋₃ haloalkyl, R¹³ is C₃₋₆ alkyl, and R^{b} is methyl or ethyl, each optionally substituted with hydroxyl or amino.

In certain embodiments, the compound of Formula (IVd), or a pharmaceutically acceptable salt thereof, has the structure:

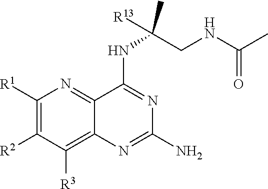

wherein R¹³ is C₃₋₆ alkyl. R¹, R² and R³ can be as defined above for Formula (J), (I), (IV), (IVa) or (IVd).

In certain embodiments, the compound of Formula (IVd), or a pharmaceutically acceptable salt thereof, has the structure:

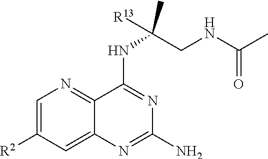

wherein R² is selected from the group consisting of hydrogen and F, and R¹³ is C₃₋₆ alkyl. In certain embodiments, R² and R¹³ can be as defined above for Formula (J), (I), (IV), or (IVa), or any combination thereof.

In certain embodiments, the compound of Formula (IVd), or a pharmaceutically acceptable salt thereof, has the structure:

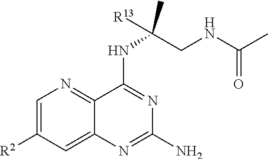

wherein R² is selected from the group consisting of hydrogen, Cl, and F, and R¹³ is C₃₋₆ alkyl. In certain embodiments, R² and R¹³ can be as defined above for Formula (J), (I), (IV), or (IVa), or any combination thereof.

In certain embodiments, the compound of Formula (IVd), or a pharmaceutically acceptable salt thereof, has the structure:

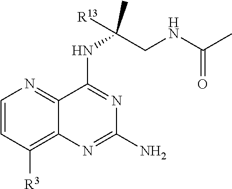

wherein R³ is selected from the group consisting of hydrogen and methyl, and R¹³ is C₃₋₆ alkyl. [In certain embodiments, the compound of Formula (J), (I), or (IV), is selected from:

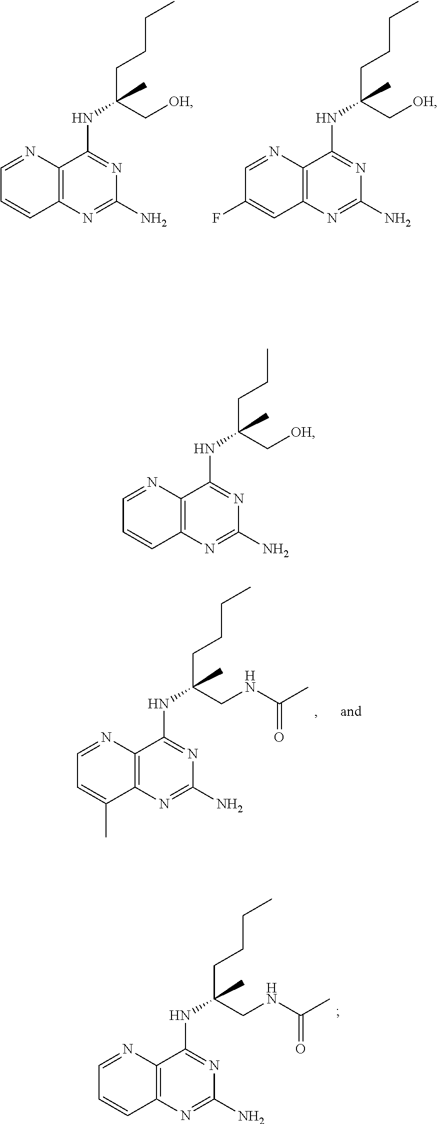

or a pharmaceutically acceptable salt thereof.

In certain embodiments, the compound of Formula (J), (I), or (IV), is selected from:

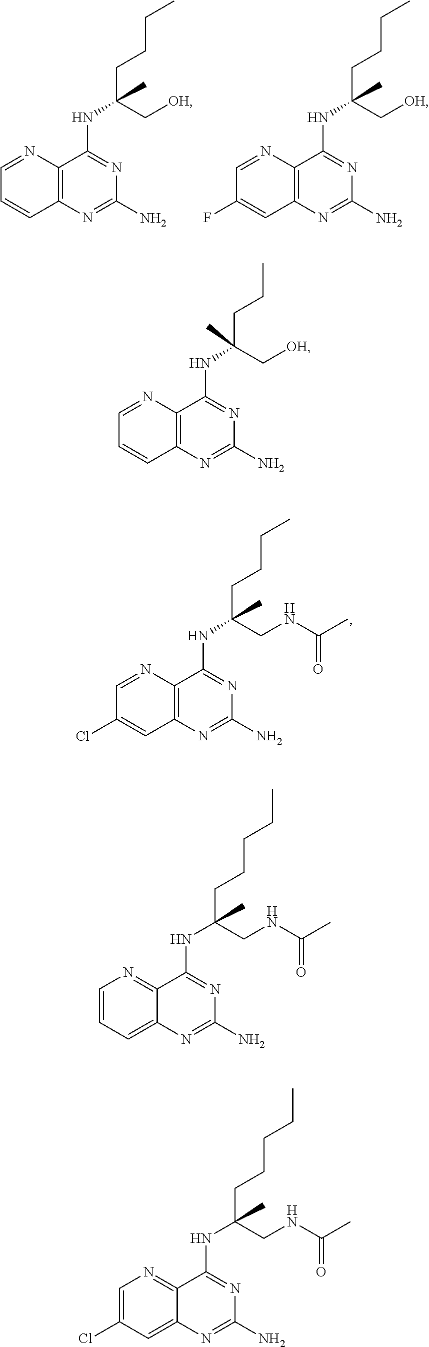

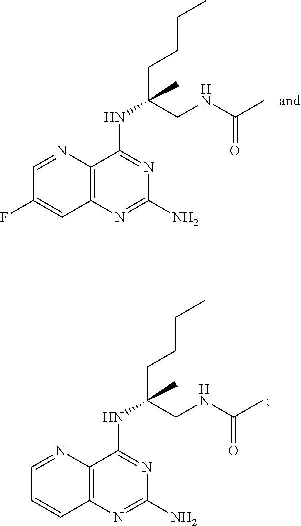

or a pharmaceutically acceptable salt thereof.

In certain embodiments, the compound of Formula (J), (I), or (IV), or a pharmaceutically acceptable salt thereof, is a compound of the following formula:

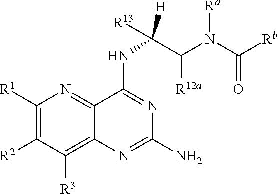

wherein R¹ is selected from the group consisting of hydrogen, halogen, C₁₋₆ alkyl, CN, and OR^{a}, wherein C₁₋₆ alkyl is optionally substituted with 1 to 5 R²⁰ groups, R² is selected from the group consisting of hydrogen, halogen, C₁₋₆ alkyl, CN, and OR^{a}, wherein C₁₋₆ alkyl optionally substituted with 1 to 5 R²⁰ groups, R³ is selected from the group consisting of hydrogen, halogen, C₁₋₆ alkyl, CN, and OR^{a}, wherein C₁₋₆ alkyl is optionally substituted with 1 to 5 R²⁰ groups, R^{12a} is selected from the group consisting of hydrogen, C₁₋₂ alkyl and C₁₋₃ haloalkyl, R¹³ is C₃₋₆ alkyl optionally substituted with 1 to 2 halogen substituents, each R²⁰ is independently selected from the group consisting of halogen, C₁₋₆haloalkyl, CN, —NR^{a}R^{b}, S(O)₁₋₂R^{a}, and OR^{a}, and each R^{a} and R^{b} is independently selected from the group consisting of hydrogen and C₁₋₃ alkyl, wherein each C₁₋₃ alkyl is optionally substituted with 1 to 3 substituents independently selected from halogen, hydroxyl, amino, and C₁₋₆ haloalkyl.

In certain embodiments, the compound of Formula (J), (I), or (IV), or a pharmaceutically acceptable salt thereof, is a compound of the following formula:

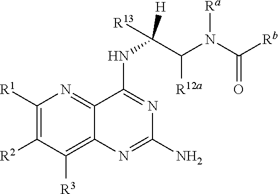

wherein R¹ is selected from the group consisting of hydrogen, halogen, and C₁₋₃ alkyl, R² is selected from the group consisting of hydrogen, halogen, and C₁₋₃alkyl, R³ is selected from the group consisting of hydrogen, halogen, and C₁₋₃ alkyl, R^{12a} is selected from the group consisting of hydrogen, C₁₋₂ alkyl and C₁₋₃haloalkyl, R¹³ is C₃₋₆ alkyl optionally substituted with 1 to 2 halogen substituents, and each R^{a} and R^{b} is independently selected from the group consisting of hydrogen and C₁₋₃ alkyl, wherein each C₁₋₃ alkyl is optionally substituted with 1 to 3 substituents independently selected from halogen, hydroxyl, amino, and C₁₋₆haloalkyl.

In certain embodiments, the compound of Formula (J), (I), or (IV), or a pharmaceutically acceptable salt thereof, is a compound of the following formula:

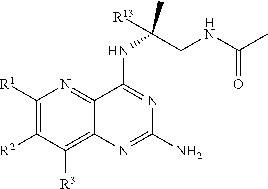

wherein R¹³ is C₃₋₆ alkyl. R¹, R² and R³ can be as defined above for Formula (J), (I), (IV), (IVa) or (IVd).

In certain embodiments of a compound of Formula (J), (I), (II), (IIa), (IIb), (III), (IIIa), or (IIIb), R¹ is hydrogen, halogen, or C₁₋₆alkyl optionally substituted with 1 to 5 R²⁰ groups. In certain embodiments of a compound of Formula (J), (I), (II), (IIa), (IIb), (III), (IIIa), (IIIb), (IV), (IVa), (Vb), or (IVd), R¹ is hydrogen, halogen, or C₁₋₆alkyl optionally substituted with 1 to 5 R²⁰ groups.

In certain embodiments of a compound of Formula (J), (I), (II), (IIa), (IIb), (III), (IIIa), or (IIIb), R¹ is hydrogen, halogen, or C₁₋₃alkyl optionally substituted with 1 to 5 halogens. In certain embodiments of a compound of Formula (J), (I), (II), (IIa), (IIb), (III), (IIIa), (IIIb), (IV), (IVa), (IVb) or (IVd), R¹ is hydrogen, halogen, or C₁₋₃alkyl optionally substituted with 1 to 5 halogens.

In certain embodiments of a compound of Formula (J), (I), (II), (IIa), (IIb), (III), (IIIa), or (IIIb), R¹ is hydrogen, Cl, CH₃, or CF₃. In certain embodiments of a compound of Formula (J), (I), (II), (IIa), (IIb), (III), (IIIa), (IIIb), (IV), (IVa), (IVb) or (IVd), R¹ is hydrogen, Cl, CH₃, or CF₃.

In certain embodiments of a compound of Formula (J), (I), (II), (IIa), (IIb), (III), (IIIa), or (IIIb), R² is hydrogen, halogen, CN, or C₁₋₆alkyl optionally substituted with 1 to 5 R²⁰ groups. In certain embodiments of a compound of Formula (J), (I), (II), (IIa), (IIb), (III), (IIIa), (IIIb), (IV), (IVa), (IVb), (IVc), or (IVd), R² is hydrogen, halogen, CN, or C₁₋₆alkyl optionally substituted with 1 to 5 R²⁰ groups.

In certain embodiments of a compound of Formula (J), (I), (II), (IIa), (IIb), (III), (IIIa), or (IIIb), R² is hydrogen, halogen, CN or C₁₋₃alkyl optionally substituted with 1 to 5 halogens. In certain embodiments of a compound of Formula (J), (I), (II), (IIa), (IIb), (III), (IIIa), (IIIb), (IV), (IVa), (IVb), (IVc), or (IVd), R² is hydrogen, halogen, CN or C₁₋₃alkyl optionally substituted with 1 to 5 halogens.

In certain embodiments of a compound of Formula (J), (I), (II), (IIa), (IIb), (III), (IIIa), or (IIIb), R² is hydrogen, CH₃, —CH₂CH₃, F, Br, Cl, or CN. In certain embodiments of a compound of Formula (J), (I), (II), (IIa), (IIb), (III), (IIIa), (IIIb), (IV), (IVa), (IVb), (IVc), or (IVd), R² is hydrogen, CH₃, —CH₂CH₃, F, Br, Cl, or CN.

In certain embodiments of a compound of Formula (J), (I), (II), (IIa), (IIb), (III), (IIIa), or (IIIb), R³ is hydrogen, halogen, or C₁₋₆alkyl optionally substituted with 1 to 5 R²⁰ groups. In certain embodiments of a compound of Formula (J), (I), (II), (IIa), (IIb), (III), (IIIa), (IIIb), (IV), (IVa), (IVb) or (d), R³ is hydrogen, halogen, or C₁₋₆alkyl optionally substituted with 1 to 5 R²⁰ groups.

In certain embodiments of a compound of Formula (J), (I), (II), (IIa), (IIb), (III), (IIIa), or (IIIb), R³ is hydrogen, halogen, or C₁₋₃alkyl optionally substituted with 1 to 5 R²⁰ groups. In certain embodiments of a compound of Formula (J), (I), (II), (IIa), (IIb), (III), (IIIa), (IIIb), (IV), (IVa), (IVb) or (IVd), R³ is hydrogen, halogen, or C₁₋₃alkyl optionally substituted with 1 to 5 R²⁰ groups.

In certain embodiments of a compound of Formula (J), (I), (II), (IIa), (IIb), (III), (IIIa), or (IIIb), R³ is hydrogen, Cl, or CH₃. In certain embodiments of a compound of Formula (J), (I), (II), (IIa), (IIb), (III), (IIIa), (IIIb), (IV), (IVa), (IVb) or (Nd), R³ is hydrogen, Cl, or CH₃.

In certain embodiments of a compound of Formula (J), R¹⁰ is hydrogen, F, Cl, or CH₃.

In certain embodiments of a compound of Formula (J), R¹⁰ is hydrogen.

In certain embodiments of a compound of Formula (J), (I), (II), (IIa), (IIb), (III), (IIIa), or (IIIb), R¹, R², and R³ are hydrogen. In certain embodiments of a compound of Formula (J), (I), (II), (IIa), (IIb), (III), (IIIa), (IIIb), (IV), (IVa), (IVb), ((IVc), or (IVd), R¹, R², and R³ are hydrogen.

In certain embodiments of a compound of Formula (J), (I), (II), (IIa), (IIb), (III), (IIIa), or (IIIb), R¹ and R³ are hydrogen and R² is F. In certain embodiments of a compound of Formula (J), (I), (II), (IIa), (IIb), (III), (IIIa), (IIIb), (IV), (IVa), (IVb), (IVc), or (IVd), R¹ and R³ are hydrogen and R² is F.

It is understood that each of the variables (e.g. R¹, R², R³, R⁴) may be combined with any other variables for Formula (J), (I), (II), (IIa) or (IIb) (e.g. R¹, R², R³, R⁴). Further, in instances describing a compound of Formula (J) or (I), it is understood that the variables also describe compounds of other formulae (e.g. Formula (II), (IIa), (IIb), (III), (IIIa), and (IIIb)) which fall within the scope of Formula (J) or (I).

It is understood that any variable for R¹ of Formula (J), (I), (II), (IIa), (IIb), (III), (IIIa), or (IIIb) may be combined with any variable of R⁴ in Formula (J), (I), (II), (IIa), (IIb), (III), (IIIa), or (IIIb), the same as if each and every combination were specifically and individually listed. For example, in one variation of Formula (J) or (I), R¹ is hydrogen, Cl, CH₃ or CF₃, and R⁴ is C₁₋₆ alkyl which is optionally substituted with 1 or 2 substituents independently selected from OH, CF₃, —C(O)OH, —C(O)OCH₃, —C(O)NH₂, SCH₃, —C(O)NHCH₃, —C(O)NHCH₂CH₂NH₂, —C(O)NHCH₂CH₂OH, —C(O)NHCH₂-pyridyl, phenyl, tetrahydrofuranyl, and cyclopropyl.

It is understood that any variable for R² of Formula (J), (I), (II), (IIa), (IIb), (III), (IIIa), or (IIIb) may be combined with any variable of R⁴ in Formula (J), (I), (II), (IIa), (IIb), (III), (IIIa), or (IIIb), the same as if each and every combination were specifically and individually listed. For example, in one variation of Formula (J) or (I), R² is hydrogen, CH₃, —CH₂CH₃, F, Br, Cl, or CN, and R⁴ is C₁₋₆ alkyl which is optionally

substituted with 1 or 2 substituents independently selected from OH,
CF₃, —C(O)OH, —C(O)OCH₃, —C(O)NH₂, SCH₃, —C(O)NHCH₃, —C(O)NHCH₂CH₂NH₂, —C(O)NHCH₂CH₂OH, —C(O)NHCH₂-pyridyl, phenyl, tetrahydrofuranyl, and cyclopropyl.

It is understood that any variable for R³ of Formula (J), (I), (II), (IIa), (IIb), (III), (IIIa), or (IIIb) may be combined with any variable of R⁴ in Formula (J), (I), (II), (IIa), (IIb), (III), (IIIa), or (IIIb), the same as if each and every combination were specifically and individually listed. For example, in one variation of Formula (J) or (I), R³ is hydrogen, Cl, or CH₃, and R⁴ is C₁₋₆ alkyl which is optionally substituted with 1 or 2 substituents independently selected from OH, CF₃, —C(O)OH, —C(O)OCH₃, —C(O)NH₂, SCH₃, —C(O)NHCH₃, —C(O)NHCH₂CH₂NH₂, —C(O)NHCH₂CH₂OH, —C(O)NHCH₂-pyridyl, phenyl, tetrahydrofuranyl, and cyclopropyl.

In certain embodiments, the compound of Formula (J) or (I), or a pharmaceutically acceptable salt thereof, has one or more features selected from:

(a) R⁴ is C₁₋₆ alkyl which is optionally substituted with 1 or 2 substituents independently selected halogen, —OR^{a}, —C(O)OR^{a}, —C(O)NR^{a}R^{b}, —SR^{a}, C₁₋₃haloalkyl, C₃₋₆cycloalkyl, 3 to 6 membered heterocyclyl and C₆₋₁₀ aryl; wherein each C₃₋₆cycloalkyl and C₆₋₁₀ aryl is optionally substituted with 1 to 3 R²¹ groups and wherein R^{a} and R^{b} are each independently hydrogen or C₁₋₄alkyl, wherein each C₁₋₄ alkyl is optionally substituted with —NH₂, OH, or pyridyl;
(b) R¹ is hydrogen, halogen, or C₁₋₆alkyl optionally substituted with 1 to 5 R²⁰ groups;
(c) R² is hydrogen, halogen, CN, or C₁₋₆alkyl optionally substituted with 1 to 5 R²⁰ groups; and
(d) R³ is hydrogen, halogen, or C₁₋₃alkyl optionally substituted with 1 to 5 R²⁰ groups.

In certain embodiments, the compound of Formula (J) or (I), or a pharmaceutically acceptable salt thereof has two or more features selected from (a)-(d), as listed above. In certain embodiments, the compound of Formula (J) or (I), or a pharmaceutically acceptable salt thereof has three or more features selected from (a)-(d), as listed above. In certain embodiments, the compound of Formula (J) or (I), or a pharmaceutically acceptable salt thereof has four features selected from (a)-(d), as listed above.

In certain embodiments, the compound of Formula (J) or (I), or a pharmaceutically acceptable salt thereof has one or more features selected from:

(e) R⁴ is C₁₋₆ alkyl which is optionally substituted with 1 or 2 substituents independently selected from OH, CF₃, —C(O)OH, —C(O)OCH₃, —C(O)NH₂, SCH₃, —C(O)NHCH₃, —C(O)NHCH₂CH₂NH₂, —C(O)NHCH₂CH₂OH, —C(O)NHCH₂-pyridyl, phenyl, tetrahydrofuranyl, and cyclopropyl.
(f) R¹ is hydrogen, halogen, or C₁₋₃alkyl optionally substituted with 1 to 5 halogens;
(g) R² is hydrogen, halogen, CN or C₁₋₃alkyl optionally substituted with 1 to 5 halogens; and
(h) R³ is hydrogen, halogen, or C₁₋₃alkyl.

In certain embodiments, the compound of Formula (J) or (I), or a pharmaceutically acceptable salt thereof has two or more features selected from (e)-(h), as listed above. In certain embodiments, the compound of Formula (J) or (I), or a pharmaceutically acceptable salt thereof has three or more features selected from (e)-(h), as listed above. In certain embodiments, the compound of Formula (J) or (I), or a pharmaceutically acceptable salt thereof has two or more features selected from (e)-(h), as listed above.

In certain embodiments, the compound of Formula (J) or (I) is selected from:

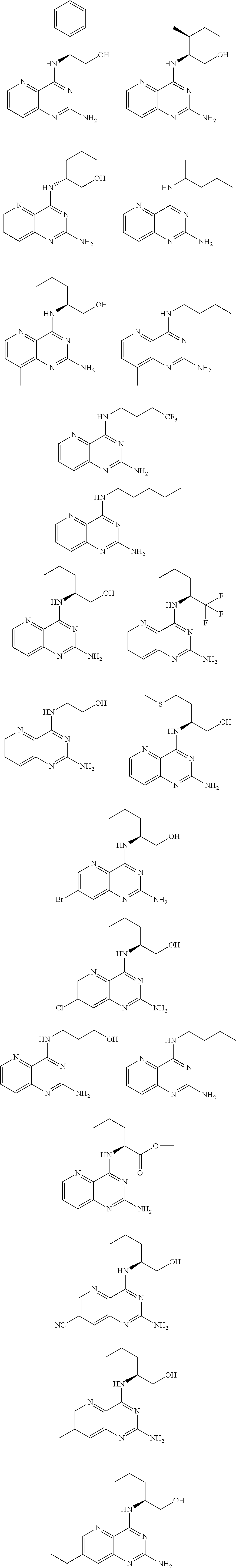

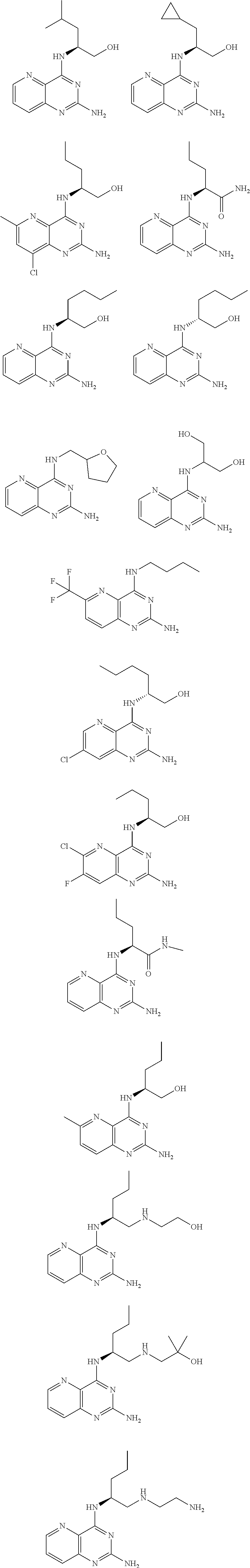

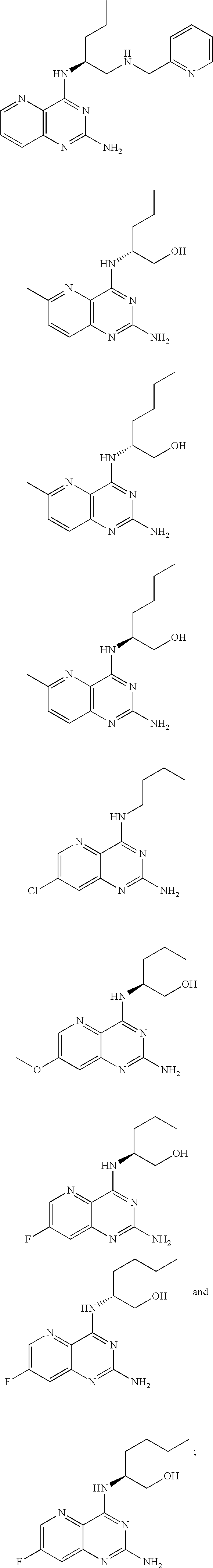

or a pharmaceutically acceptable salt thereof.

In certain embodiments, the compound of Formula (J) or (I) is selected from:

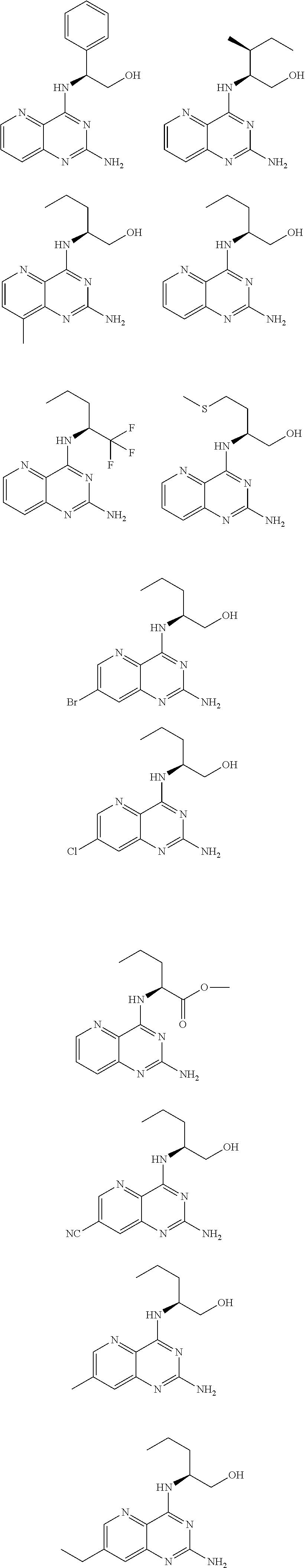

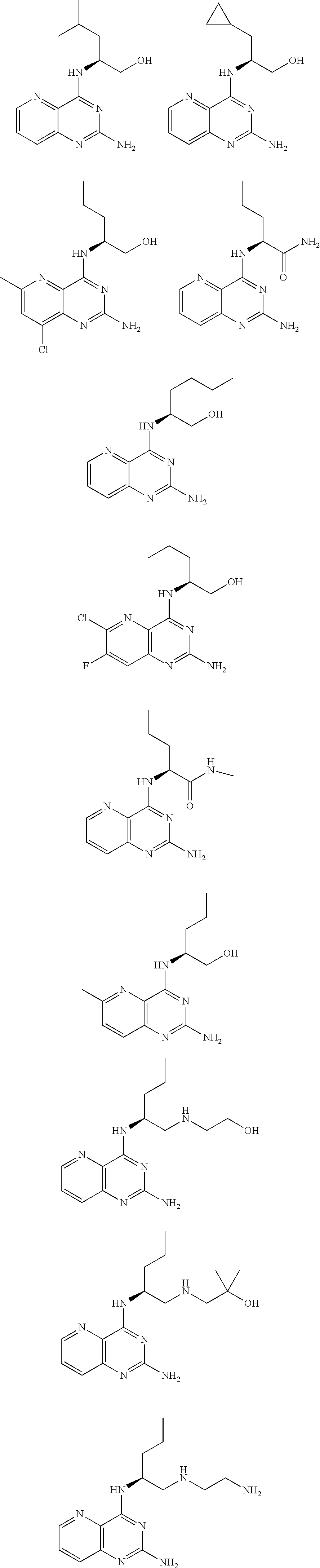

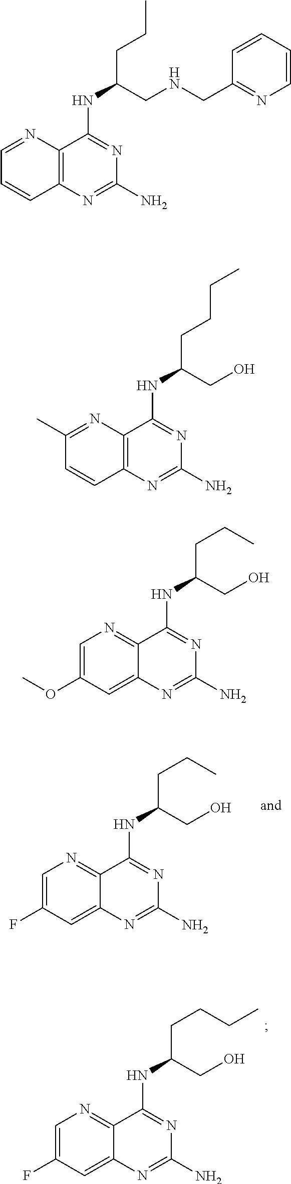

or a pharmaceutically acceptable salt thereof.

In certain embodiments, the compound of Formula (J) or (I) is selected from:

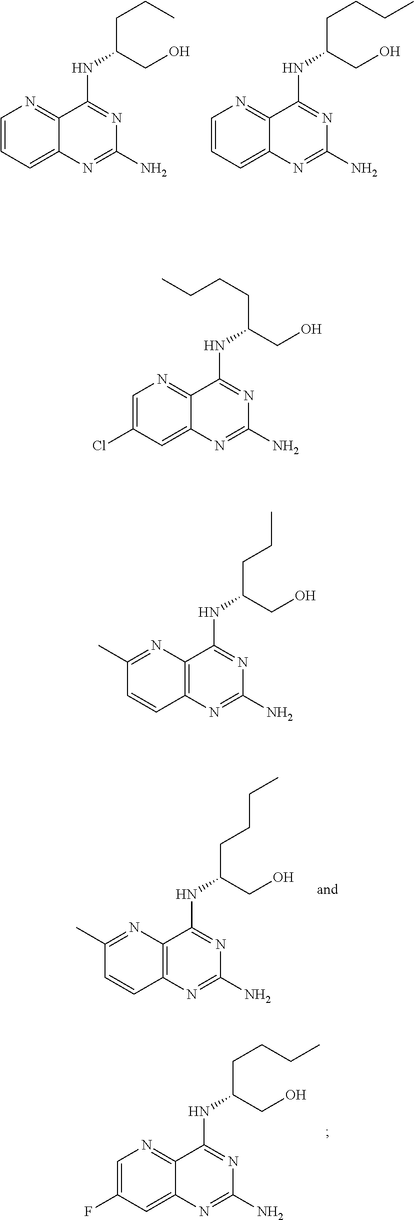 [SMILES: CCC[C@H](CO)Nc1nc(N)nc2cccnc12 ; CCCC[C@H](CO)Nc1nc(N)nc2cccnc12 ; CCCC[C@H](CO)Nc1nc(N)nc2cc(Cl)cnc12 ; CCC[C@H](CO)Nc1nc(N)nc2ccc(C)nc12 ; CCCC[C@H](CO)Nc1nc(N)nc2ccc(C)nc12 ; CCCC[C@H](CO)Nc1nc(N)nc2cc(F)cnc12]

or a pharmaceutically acceptable salt thereof.

In certain embodiments, the compound of Formula (J) is selected from:

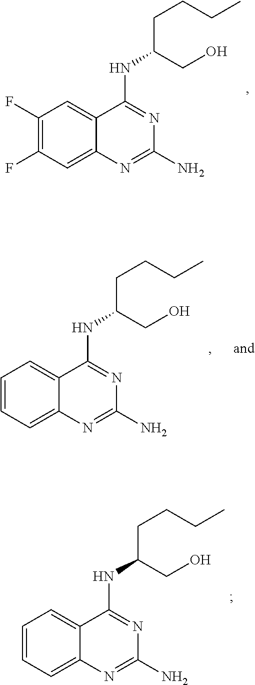 [SMILES: CCCC[C@H](CO)Nc1nc(N)nc2cc(F)c(F)cc12 ; CCCC[C@H](CO)Nc1nc(N)nc2ccccc12 ; CCCC[C@@H](CO)Nc1nc(N)nc2ccccc12]

or a pharmaceutically acceptable salt thereof.

In certain embodiments, the compound of Formula (J) or (I) is selected from:

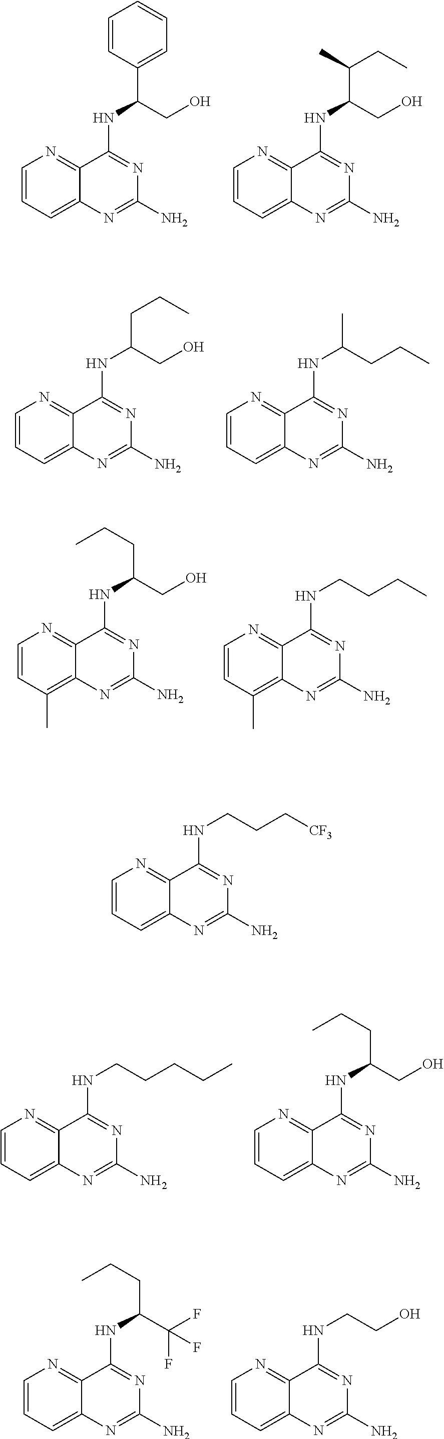

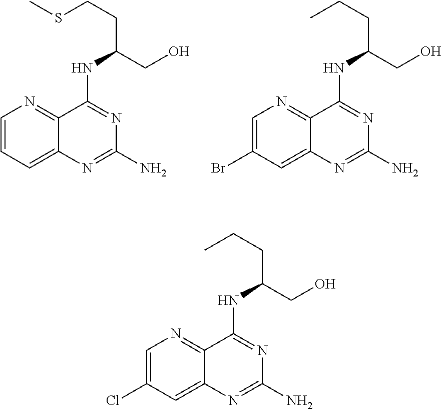 [SMILES: CSCC[C@@H](CO)Nc1nc(N)nc2cccnc12 ; CCC[C@@H](CO)Nc1nc(N)nc2cc(Br)cnc12 ; CCC[C@@H](CO)Nc1nc(N)nc2cc(Cl)cnc12]

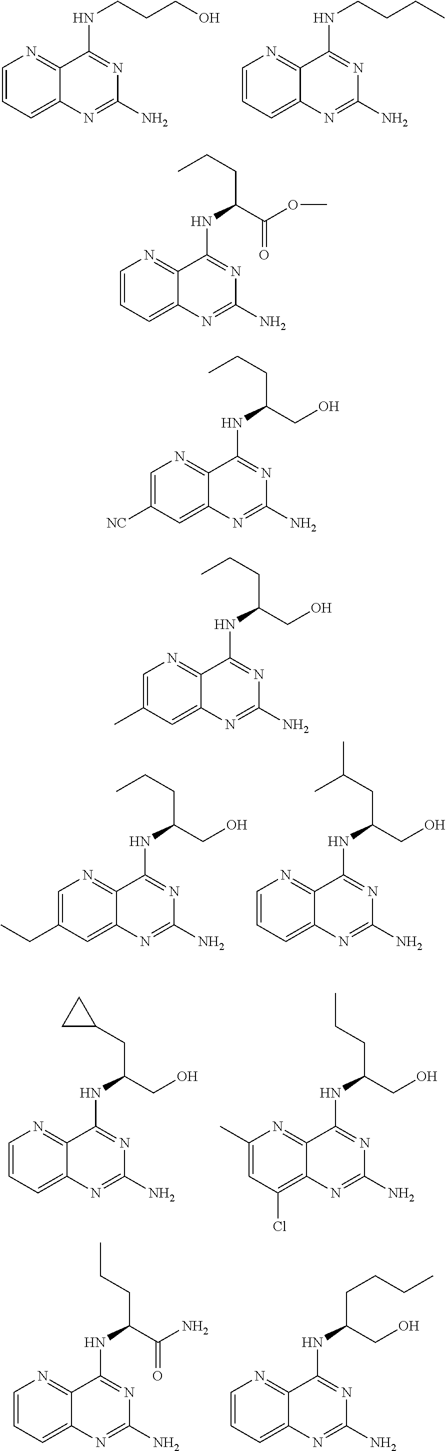

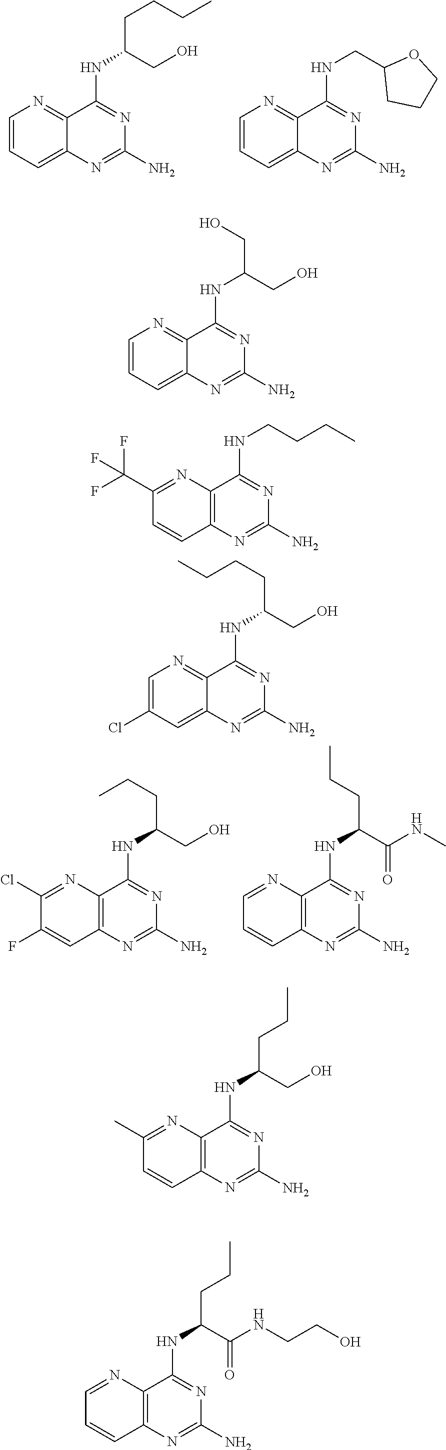

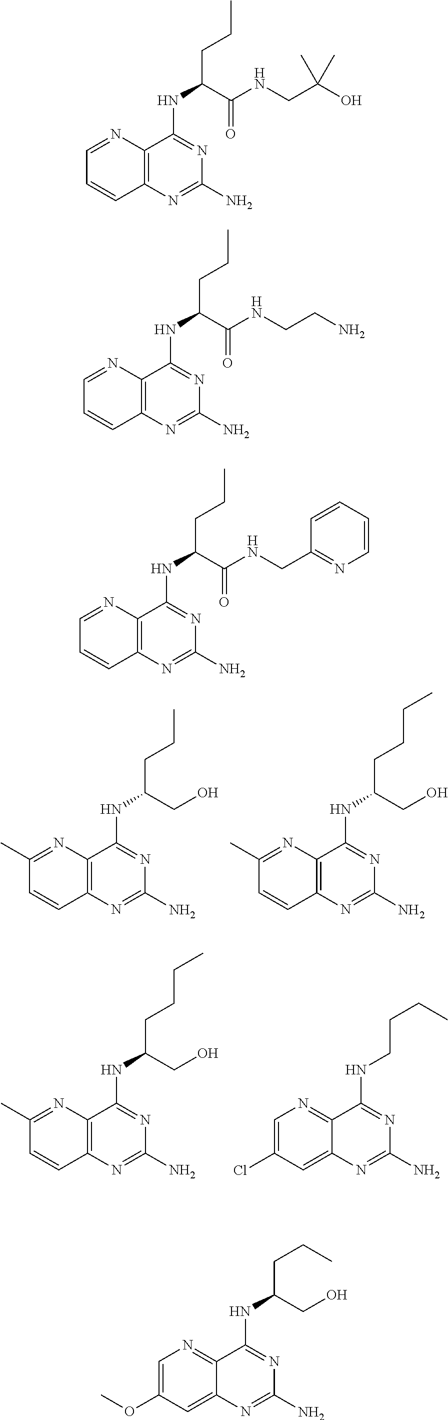

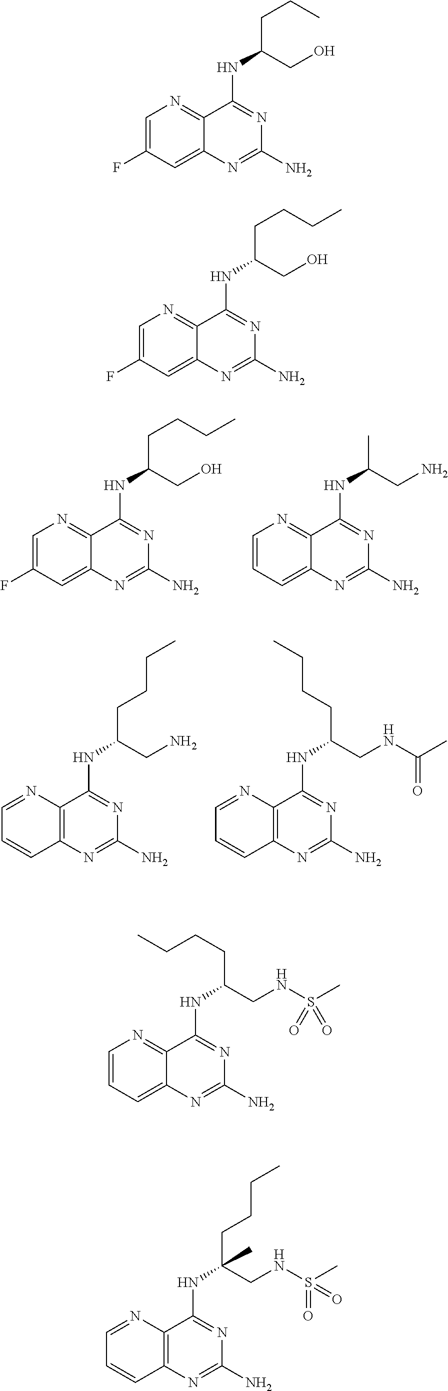

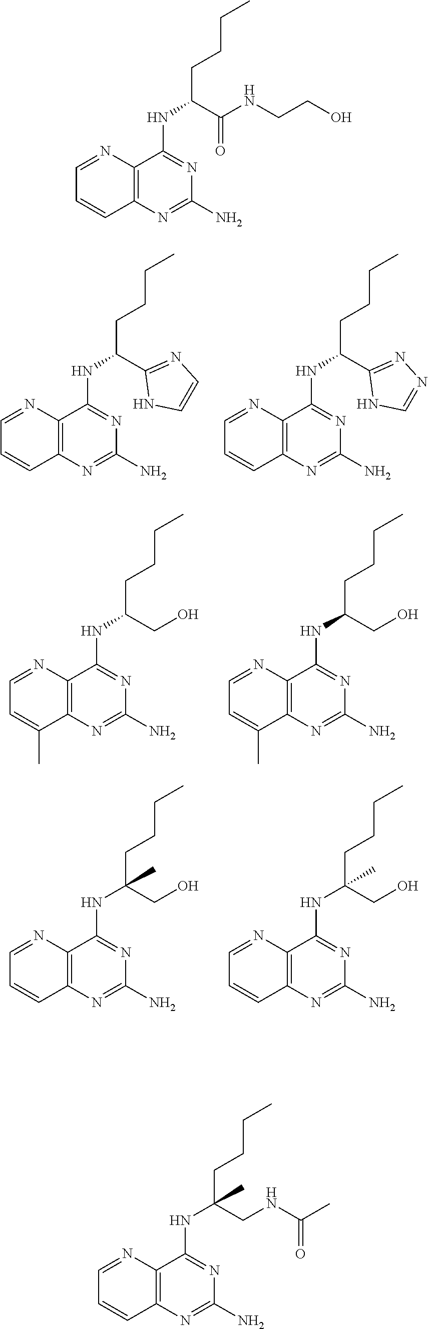

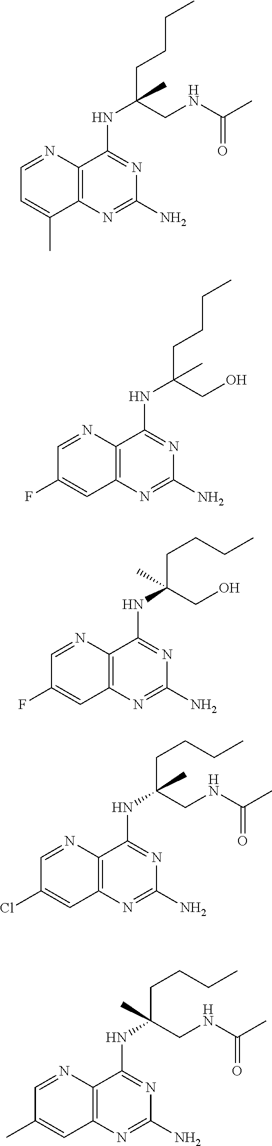

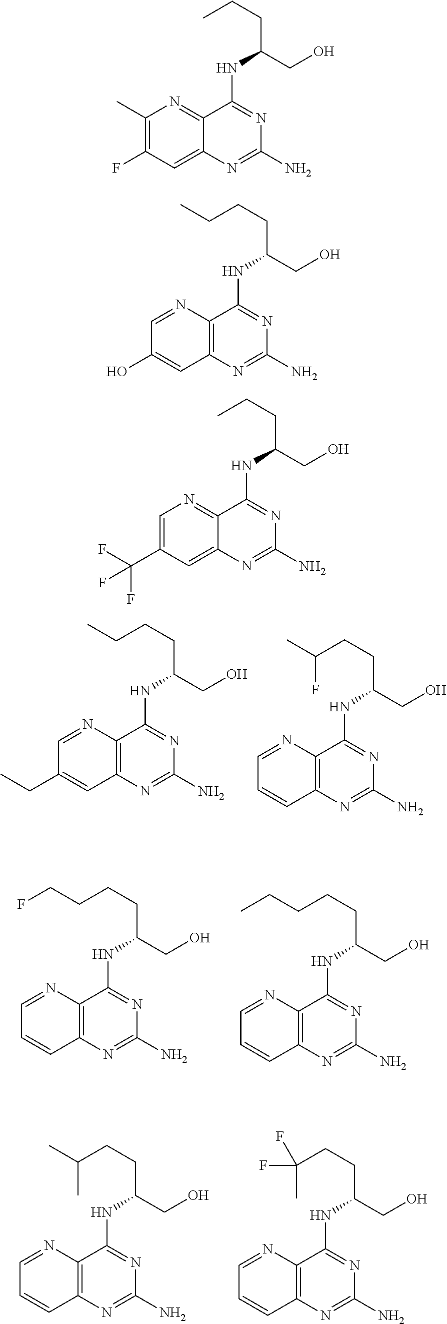

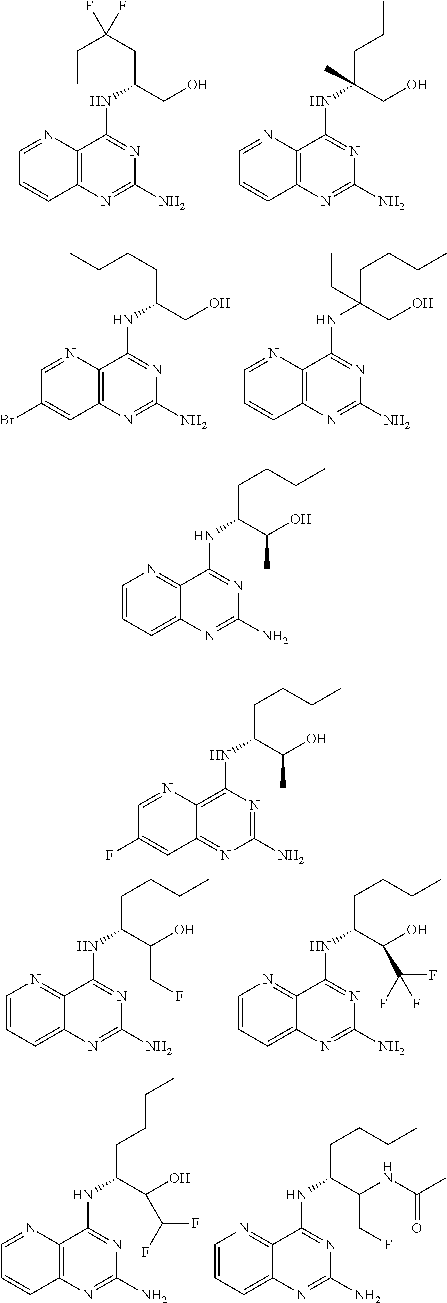

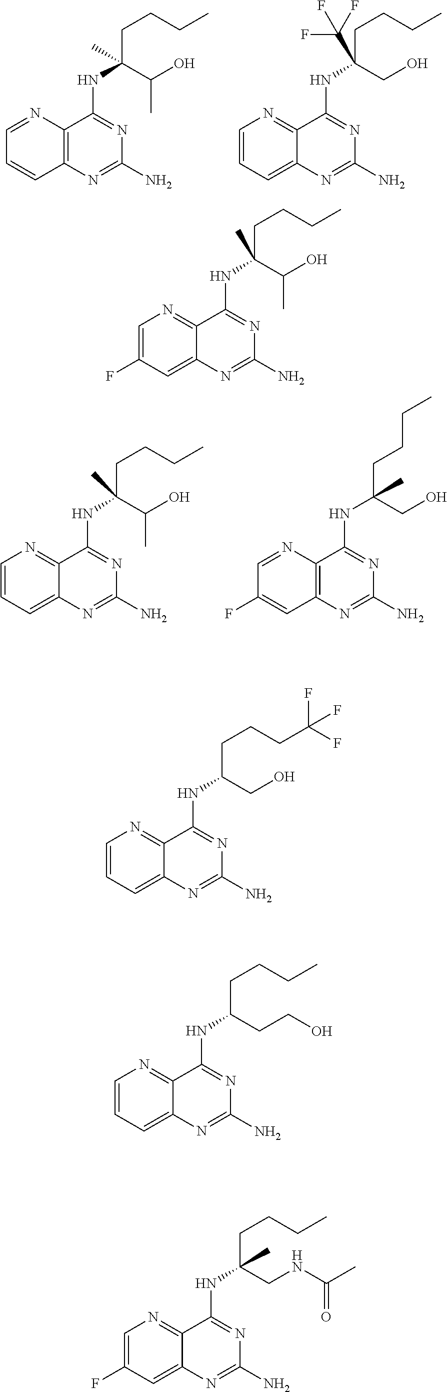

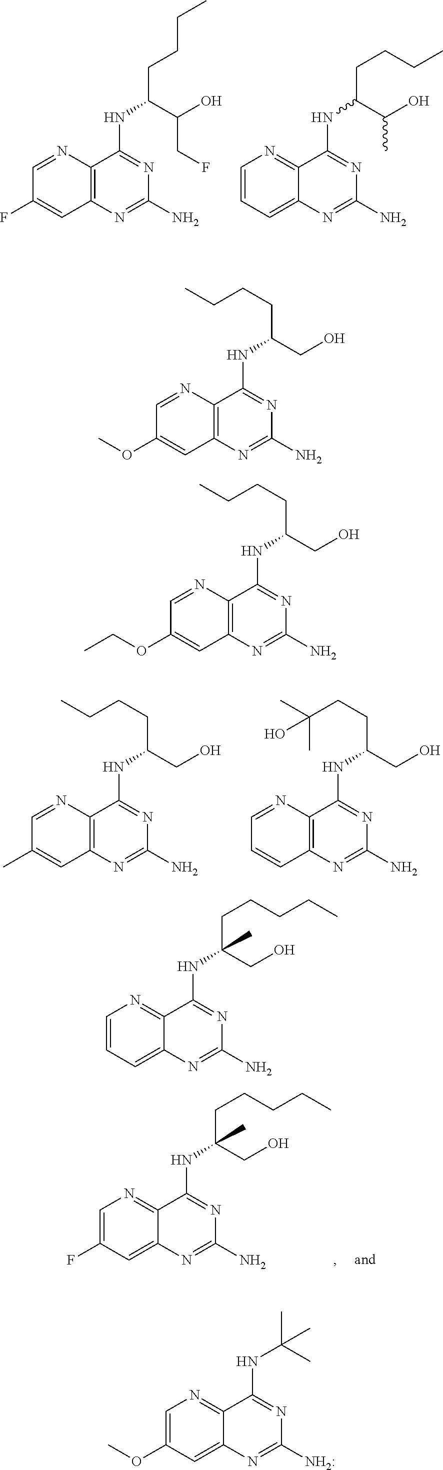

or a pharmaceutically acceptable salt thereof.

In certain embodiments, the compound of Formula (J), (I), (IV), or (IVa) is selected from:

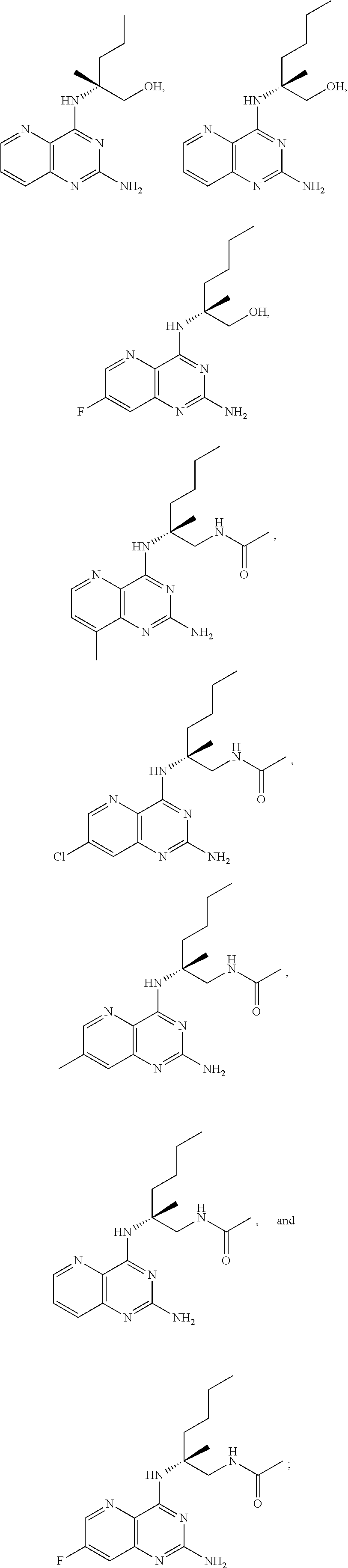

or a pharmaceutically acceptable salt thereof.

In certain embodiments, the compound of Formula (J), (I), (IV), or (IVa) is selected from:

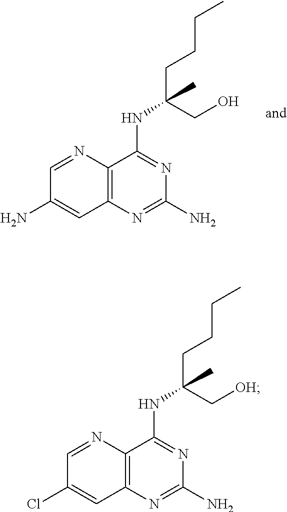

or a pharmaceutically acceptable salt thereof.

In certain embodiments, the compound of Formula (J), (I), (IV), or (IVa) is selected from:

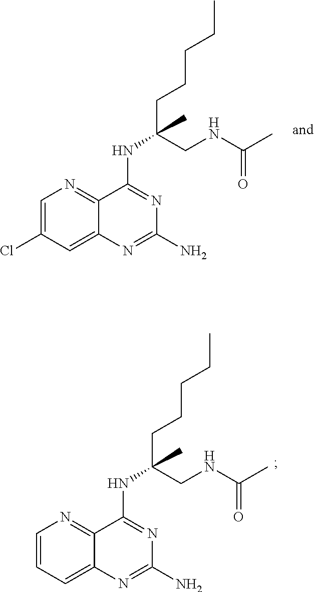

or a pharmaceutically acceptable salt thereof.

In certain embodiments, the compound of Formula (J), (I), (IV), or (IVa) is selected from:

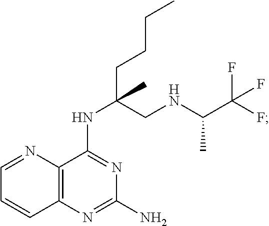 [SMILES: CCCC[C@](C)(CN[C@@H](C)C(F)(F)F)Nc1nc(N)nc2cccnc12]

or a pharmaceutically acceptable salt thereof.

In certain embodiments, the compound of Formula (J), (I), (IV), or (IVa) is selected from:

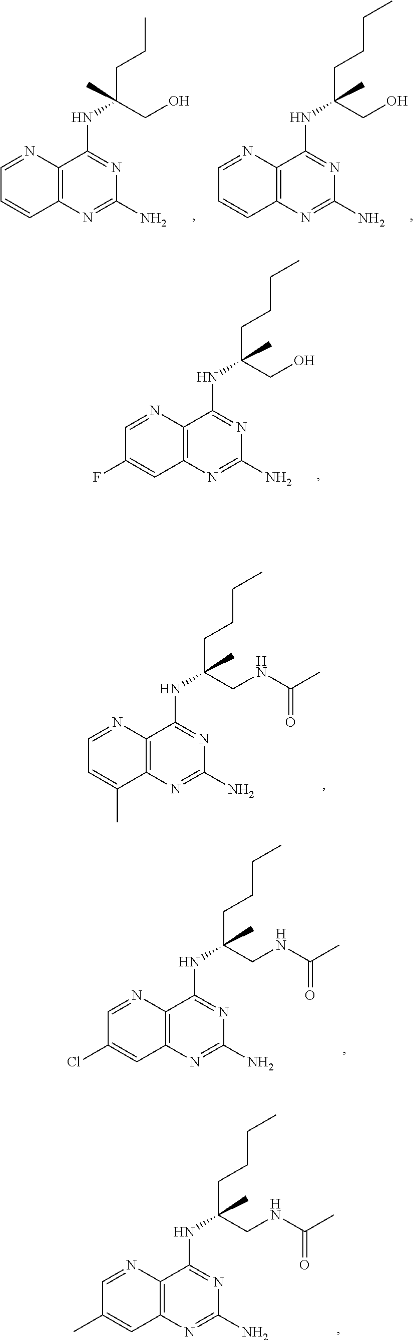

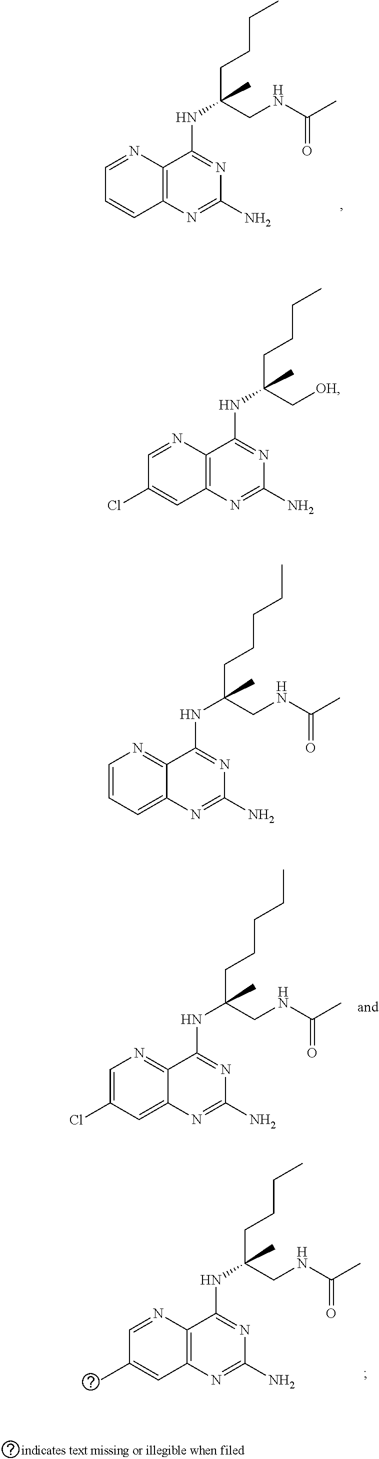

or a pharmaceutically acceptable salt thereof.

As used herein, “a compound of Formula (I)” includes compounds for Formula (II), (IIa), (IIb), (III), (IIIa), (IIIb), (IV), (IVa), (IVb), (IVc), or (IVd).

### Compositions:

In certain embodiments, the present disclosure provides a pharmaceutical composition comprising a compound of the present disclosure (e.g. a compound of Formula (J), (I), (II), (IIa), (IIb), (III), (IIIa), (IIIb), (IV), (IVa), (IVb), (IVc), or (IVd)), or a pharmaceutically acceptable salt thereof, and a pharmaceutically acceptable excipient.

In certain embodiments, the pharmaceutical composition comprises one or more additional therapeutic agent, as more fully set forth below.

Pharmaceutical compositions comprising the compounds disclosed herein, or pharmaceutically acceptable salts thereof, may be prepared with one or more pharmaceutically acceptable excipients which may be selected in accord with ordinary practice. Tablets may contain excipients including glidants, fillers, binders and the like. Aqueous compositions may be prepared in sterile form, and when intended for delivery by other than oral administration generally may be isotonic. All compositions may optionally contain excipients such as those set forth in the Rowe et al, Handbook of Pharmaceutical Excipients, 6^{th} edition, American Pharmacists Association, 2009.

Excipients can include ascorbic acid and other antioxidants, chelating agents such as EDTA, carbohydrates such as dextrin, hydroxyalkylcellulose,
hydroxyalkylmethylcellulose, stearic acid and the like. In certain embodiments, the composition is provided as a solid dosage form, including a solid oral dosage form.
The compositions include those suitable for various administration routes, including oral administration. The compositions may be presented in unit dosage form and may be prepared by any of the methods well known in the art of pharmacy. Such methods include the step of bringing into association the active ingredient (e.g., a compound of the present disclosure or a pharmaceutical salt thereof) with one or more pharmaceutically acceptable excipients. The compositions may be prepared by uniformly and intimately bringing into association the active ingredient with liquid excipients or finely divided solid excipients or both, and then, if necessary, shaping the product. Techniques and formulations generally are found in Remington: The Science and Practice of Pharmacy, 21^{st} Edition, Lippincott Williams and Wilkins, Philadelphia, Pa., 2006.

Compositions described herein that are suitable for oral administration may be presented as discrete units (a unit dosage form) including but not limited to capsules, cachets or tablets each containing a predetermined amount of the active ingredient. In one embodiment, the pharmaceutical composition is a tablet.

Pharmaceutical compositions disclosed herein comprise one or more compounds disclosed herein, or a pharmaceutically acceptable salt thereof, together with a pharmaceutically acceptable excipient and optionally other therapeutic agents.

Pharmaceutical compositions containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more excipients including sweetening agents, flavoring agents, coloring agents and preserving agents, in order to provide a palatable preparation. Tablets containing the active ingredient in admixture with non-toxic pharmaceutically acceptable excipients which are suitable for manufacture of tablets are acceptable. These excipients may be, for example, inert diluents, such as calcium or sodium carbonate, lactose, lactose monohydrate, croscarmellose sodium, povidone, calcium or sodium phosphate;
granulating and disintegrating agents, such as maize starch, or alginic acid; binding agents, such as cellulose, microcrystalline cellulose, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

The amount of active ingredient that may be combined with the inactive ingredients to produce a dosage form may vary depending upon the intended treatment subject and the particular mode of administration. For example, in some embodiments, a dosage form for oral administration to humans may contain approximately 1 to 1000 mg of active material formulated with an appropriate and convenient amount of a pharmaceutically acceptable excipient. In certain embodiments, the pharmaceutically

acceptable excipient varies from about 5 to about 95% of the total compositions (weight:weight).

In certain embodiments, a composition comprising a compound of the present disclosure (e.g. a compound of Formula (J), (I), (II), (IIa), (IIb), (III), (IIIa), (IIIb), (IV), (IVa), (IVb), (IVc), or (IVd)), or a pharmaceutically acceptable salt thereof in one variation does not contain an agent that affects the rate at which the active ingredient is metabolized. Thus, it is understood that compositions comprising a compound of the present disclosure in one aspect do not comprise an agent that would affect (e.g., slow, hinder or retard) the metabolism of a compound of the present disclosure or any other active ingredient administered separately, sequentially or simultaneously with a compound of the present disclosure. It is also understood that any of the methods, kits, articles of manufacture and the like detailed herein in one aspect do not comprise an agent that would affect (e.g., slow, hinder or retard) the metabolism of a compound of the present disclosure or any other active ingredient administered separately, sequentially or simultaneously with a compound of the present disclosure.

### IV. Methods

The present disclosure provides for methods of treating diseases or conditions that are responsive to the modulation of toll-like receptors (e.g. TLR-8 receptors). While not wishing to be bound by any one theory, the presently disclosed compounds are believed to modulate TLR-8 receptors as agonists. As is understood by those of skill in the art, modulators of TLR-8 may, to some degree, modulate other toll-like receptors (e.g. TLR-7). As such, in certain embodiments, the compounds disclosed herein may also modulate TLR-7 to a measureable degree. In certain embodiments, those compounds that modulate TLR-8 to a higher degree than TLR-7 are considered selective modulators of TLR-8. Exemplary methods of measuring the each compounds respective modulation of TLR-7 and TLR-8 are described in the Examples provided herein. In certain embodiments, the compounds disclosed herein are selective modulators of TLR-8.

In certain embodiments, a method of modulating TLR-8 is provided, comprising administering a compound of the present disclosure, or a pharmaceutically acceptable salt thereof, to an individual (e.g. a human).

In certain embodiments, a method of modulating TLR-8 in vitro is provided.

In certain embodiments, the present disclosure provides a compound of the present disclosure, or a pharmaceutically acceptable salt thereof, for use as a research tool, e.g., for use in identifying modulators of TLR-8

In certain embodiments, the present disclosure provides methods for the treatment or prevention of diseases or conditions in an individual (e.g. a human) in need thereof, comprising administering a compound of the present disclosure or a

pharmaceutically acceptable salt thereof. In certain embodiments, the methods comprise administering one or more additional therapeutic agents. Treatment with a compound of the present disclosure typically results in the stimulation of an immune response to the particular disease or condition being treated. Diseases or conditions contemplated by the present disclosure include those affected by the modulation of toll-like receptors (e.g. TLR-8). In certain embodiments, a method of treating or preventing a disease or condition responsive to the modulation of TLR-8 is provided, comprising administering to a human a therapeutically effective amount of a compound of the present disclosure, or a pharmaceutically acceptable salt thereof. Exemplary diseases, disorders and conditions include but are not limited to conditions involving autoimmunity, inflammation, allergy, asthma, graft rejection, graft versus host disease (GvHD), infectious diseases, cancer, and immunodeficiency.

In certain embodiments, infectious diseases include diseases such as hepatitis A, hepatitis B (HBV), hepatitis C (HCV), hepatitis D (HDV), HIV, human

papillomavirus (HPV), respiratory syncytial virus (RSV), severe acute respiratory syndrome (SARS), influenza, parainfluenza, cytomegalovirus, dengue, herpes simplex virus-1, herpes simplex virus-2, leishmania infection, and respiratory syncytial virus. In certain embodiments, infectious diseases include diseases such as hepatitis A, hepatitis B (HBV), hepatitis D (HDV), HIV, human papillomavirus (HPV), respiratory syncytial virus (RSV), severe acute respiratory syndrome (SARS), influenza, parainfluenza, cytomegalovirus, dengue, herpes simplex virus-1, herpes simplex virus-2, leishmania infection, and respiratory syncytial virus.

In certain embodiments, a method of treating or preventing a viral infection is provided, comprising administering to an individual (e.g. a human) a therapeutically effective amount a compound of the present disclosure, or a pharmaceutically acceptable salt thereof. In one embodiment, the method can be used to induce an immune response against multiple epitopes of a viral infection in a human. Induction of an immune response against viral infection can be assessed using any technique that is known by those of skill in the art for determining whether an immune response has occurred. Suitable methods of detecting an immune response for the present disclosure include, among others, detecting a decrease in viral load or antigen in a subject's serum, detection of IFN-gamma-secreting peptide specific T cells, and detection of elevated levels of one or more liver enzymes, such as alanine transferase (ALT) and aspartate transferase (AST). In one embodiment, the detection of IFN-gamma-secreting peptide specific T cells is accomplished using an ELISPOT assay. Another embodiment includes reducing the viral load associated with HBV infection, including a reduction as measured by PCR testing.

In certain embodiments, the present invention provides a method for enhancing the efficacy of a vaccine by co-administering with the vaccine, a therapeutically effective amount of a compound of the present disclosure, or a pharmaceutically acceptable salt thereof, to an individual (e.g. a human). In certain embodiments, the compound of the present disclosure or a pharmaceutically acceptable salt thereof, may be co-administered with a vaccine to boost the immune response by allowing the production of a higher amount of antibodies or by allowing a longer lasting protection. In certain embodiments, the compounds of the present disclosure, or a pharmaceutically acceptable salt thereof, may be used as vaccine adjuvants to increase the efficacy and response to the immunization with a particular antigen. In certain embodiments, co-administering the compounds of the present disclosure, or a pharmaceutically acceptable salt thereof, with a vaccine, may influence the way a vaccine's antigen is presented to the immune system and enhance the vaccine's efficacy.

In certain embodiments, a compound of the present disclosure, or a pharmaceutically acceptable salt thereof, for use in medical therapy is provided. In certain embodiments, a compound of the present disclosure or a pharmaceutically acceptable salt thereof, for use in treating or preventing a disease or condition responsive to the modulation of TLR-8, is provided. In certain embodiments, the disease or condition is a viral infection as set forth herein.

In certain embodiments, the use of a compound of the present disclosure, or a pharmaceutically acceptable salt thereof, for the manufacture of a medicament for treating or preventing a disease or condition responsive to the modulation of TLR-8, is provided.

In certain embodiments, the present disclosure also provides methods for treating a hepatitis B viral infection, comprising administering to an individual (e.g. a human) infected with hepatitis B virus a therapeutically effective amount a compound of the present disclosure or a pharmaceutically acceptable salt thereof. Typically, the individual is suffering from a chronic hepatitis B infection, although it is within the scope of the present disclosure to treat people who are acutely infected with HBV.

The present disclosure also provides methods for treating a hepatitis C viral infection, comprising administering to an individual (e.g. a human) infected with hepatitis C virus a therapeutically effective amount a compound of the present disclosure or a pharmaceutically acceptable salt thereof. Typically, the individual is suffering from a chronic hepatitis C infection, although it is within the scope of the present disclosure to treat people who are acutely infected with HCV.

Treatment of HBV or HCV in accordance with the present disclosure typically results in the stimulation of an immune response against HBV or HCV in an individual (e.g. a human) being infected with HBV or HCV, respectively, and a consequent reduction in the viral load of HBV or HCV in the infected individual. Examples of immune responses include production of antibodies (e.g., IgG antibodies) and/or production of cytokines, such as interferons, that modulate the activity of the immune system. The immune system response can be a newly induced response, or can be boosting of an existing immune response. In particular, the immune system response can be seroconversion against one or more HBV or HCV antigens.

As described more fully herein, compounds of the present disclosure can be administered with one or more additional therapeutic agent(s) to an individual (e.g. a human) infected with HBV or HCV. The additional therapeutic agent(s) can be administered to the infected individual (e.g. a human) at the same time as a compound of the present disclosure or before or after administration of a compound of the present disclosure. For example, in certain embodiments, when used to treat or prevent HCV, a compound of the present disclosure may be administered with one or more additional therapeutic agent(s) selected from the group consisting of interferons, ribavirin or its analogs, HCV NS3 protease inhibitors, HCV NS4 protease inhibitors, HCV NS3/NS4 protease inhibitors, alpha-glucosidase 1 inhibitors, hepatoprotectants, nucleoside or nucleotide inhibitors of HCV NS5B polymerase, non-nucleoside inhibitors of HCV NS5B polymerase, HCV NS5A inhibitors, TLR-7 agonists, cyclophilin inhibitors, HCV IRES inhibitors, pharmacokinetic enhancers, and other drugs for treating HCV, or mixtures thereof. Specific examples are more fully described below.

Further, in certain embodiments, when used to treat or prevent HBV, a compound of the present disclosure may be administered with one or more additional therapeutic agent(s) selected from the group consisting of HBV DNA polymerase inhibitors, toll-like receptor 7 modulators, toll-like receptor 8 modulators, Toll-like receptor 7 and 8 modulators, Toll-like receptor 3 modulators, interferon alpha ligands, HBsAg inhibitors, compounds targeting HbcAg, cyclophilin inhibitors, HBV therapeutic vaccines, HBV prophylactic vaccines, HBV viral entry inhibitors, NTCP inhibitors, antisense oligonucleotide targeting viral mRNA, short interfering RNAs (siRNA), hepatitis B virus E antigen inhibitors, HBx inhibitors, cccDNA inhibitors, HBV antibodies including HBV antibodies targeting the surface antigens of the hepatitis B virus, thymosin agonists, cytokines, nucleoprotein inhibitors (HBV core or capsid protein inhibitors), stimulators of retinoic acid-inducible gene 1, stimulators of NOD2, recombinant thymosin alpha-1 and hepatitis B virus replication inhibitors, and combinations thereof. Specific examples are more fully described below.

In certain embodiments, the present disclosure provides a method for ameliorating a symptom associated with an HBV infection or HCV infection, wherein the method comprises administering to an individual (e.g. a human) infected with hepatitis B virus or hepatitis C virus a therapeutically effective amount of a compound of the present disclosure, or a pharmaceutically acceptable salt thereof, wherein the therapeutically effective amount is sufficient to ameliorate a symptom associated with the HBV infection or HCV infection. Such symptoms include the presence of HBV virus particles (or HCV virus particles) in the blood, liver inflammation, jaundice, muscle aches, weakness and tiredness.

In certain embodiments, the present disclosure provides a method for reducing the rate of progression of a hepatitis B viral infection or a hepatitis C virus infection, in an individual (e.g. a human), wherein the method comprises administering to an individual (e.g. a human) infected with hepatitis B virus or hepatitis C virus a therapeutically effective amount of a compound of the present disclosure, or a pharmaceutically acceptable salt thereof, wherein the therapeutically effective amount is sufficient to reduce the rate of progression of the hepatitis B viral infection or hepatitis C viral infection. The rate of progression of the infection can be followed by measuring the amount of HBV virus particles or HCV virus particles in the blood.

In certain embodiments, the present disclosure provides a method for reducing the viral load associated with HBV infection or HCV infection, wherein the method comprises administering to an individual (e.g. a human) infected with HBV or HCV a therapeutically effective amount of a compound of the present disclosure, or a pharmaceutically acceptable salt thereof, wherein the therapeutically effective amount is sufficient to reduce the HBV viral load or the HCV viral load in the individual.

In certain embodiments, the present disclosure provides a method of inducing or boosting an immune response against hepatitis B virus or hepatitis C virus in an individual (e.g. a human), wherein the method comprises administering a therapeutically effective amount of a compound of the present disclosure, or a pharmaceutically acceptable salt thereof, to the individual, wherein a new immune response against hepatitis B virus or hepatitis C virus is induced in the individual, or a preexisting immune response against hepatitis B virus or hepatitis C virus is boosted in the individual. Seroconversion with respect to HBV or HCV can be induced in the individual. Examples of immune responses include production of antibodies, such as IgG antibody molecules, and/or production of cytokine molecules that modulate the activity of one or more components of the human immune system.

In certain embodiments, an immune response can be induced against one or more antigens of HBV or HCV. For example, an immune response can be induced against the HBV surface antigen (HBsAg), or against the small form of the HBV surface antigen (small S antigen), or against the medium form of the HBV surface antigen (medium S antigen), or against a combination thereof. Again by way of example, an immune response can be induced against the HBV surface antigen (HBsAg) and also against other HBV-derived antigens, such as the core polymerase or x-protein.

Induction of an immune response against HCV or HBV can be assessed using any technique that is known by those of skill in the art for determining whether an immune response has occurred. Suitable methods of detecting an immune response for the present disclosure include, among others, detecting a decrease in viral load in a individual's serum, such as by measuring the amount of HBV DNA or HCV DNA in a subject's blood using a PCR assay, and/or by measuring the amount of anti-HBV antibodies, or anti-HCV antibodies, in the subject's blood using a method such as an ELISA.

In certain embodiments, a compound of a compound of the present disclosure (e.g. a compound of Formula (I)), or a pharmaceutically acceptable salt thereof, for use in treating or preventing a HBV infection is provided. In certain embodiments, a compound of the present disclosure (e.g. a compound of Formula (I)), or a

pharmaceutically acceptable salt thereof, for use in treating or preventing a HCV infection is provided. In certain embodiments, a compound of the present disclosure (e.g. a compound of Formula (I)), or a pharmaceutically acceptable salt thereof, for the manufacture of a medicament for treating or preventing a HBV infection is provided. In certain embodiments, a compound of the present disclosure (e.g. a compound of Formula (I)), or a pharmaceutically acceptable salt thereof, for the manufacture of a medicament for treating or preventing a HCV infection is provided.

In certain embodiments, the present disclosure also provides methods for treating a Retroviridae viral infection (e.g., an HIV viral infection) in an individual (e.g., a human), comprising administering a compound of the present disclosure, or a pharmaceutically acceptable salt thereof, to the individual.

In certain embodiments, the present disclosure also provides methods for treating a HIV infection (e.g a HIV-1 infection), comprising administering to an individual (e.g. a human) infected with HIV virus a therapeutically effective amount of a compound of the present disclosure, or a pharmaceutically acceptable salt thereof. In certain embodiments, the individual in need thereof is a human who has been infected with HIV. In certain embodiments, the individual in need thereof is a human who has been infected with HIV but who has not developed AIDS. In certain embodiments, the individual in need thereof is an individual at risk for developing AIDS. In certain embodiments, the individual in need thereof is a human who has been infected with HIV and who has developed AIDS.

In certain embodiments, a method for treating or preventing an HIV viral infection in an individual (e.g., a human), comprising administering a compound of the present disclosure, or a pharmaceutically acceptable salt thereof, to the individual is provided.

In certain embodiments, a method for inhibiting the replication of the HIV virus, treating AIDS or delaying the onset of AIDS in an individual (e.g., a human), comprising administering a compound of the present disclosure, or a pharmaceutically acceptable salt thereof, to the individual is provided.

In certain embodiments, a method for preventing an HIV infection in an individual (e.g., a human), comprising administering a compound of the present disclosure, or a pharmaceutically acceptable salt thereof, to the individual is provided. In certain embodiments, the individual is at risk of contracting the HIV virus, such as an individual who has one or more risk factors known to be associated with of contracting the HIV virus.

In certain embodiments, a method for treating an HIV infection in an individual (e.g., a human), comprising administering a compound of the present disclosure, or a pharmaceutically acceptable salt thereof, to the individual is provided.

In certain embodiments, a method for treating an HIV infection in an individual (e.g., a human), comprising administering to the individual in need thereof a

therapeutically effective amount of a compound of the present disclosure, or a pharmaceutically acceptable salt thereof, in combination with a therapeutically effective amount of one or more additional therapeutic agents selected from the group consisting of HIV protease inhibiting compounds, HIV non-nucleoside inhibitors of reverse transcriptase, HIV nucleoside inhibitors of reverse transcriptase, HIV nucleotide inhibitors of reverse transcriptase, HIV integrase inhibitors, gp41 inhibitors, CXCR4 inhibitors, gp120 inhibitors, CCR5 inhibitors, capsid polymerization inhibitors, and other drugs for treating HIV, and combinations thereof is provided.

In certain embodiments, a compound of the present invention is administered to a patient where active HIV gene expression has been suppressed by administration of antiretroviral therapy (including combination antiretroviral therapy” or “cART”).

In certain embodiments, a method of reducing the latent HIV reservoir in a human infected with HIV is provided, the method comprising administering to the human a pharmaceutically effective amount of a compound of the present disclosure. In certain embodiments, the method further comprises administering one or more anti-HIV agents. In certain embodiments, the method further comprises administering

antiretroviral therapy (including combination antiretroviral therapy” or “cART”). In certain embodiments, active HIV gene expression in the human has been suppressed by administration of antiretroviral therapy (including combination antiretroviral therapy” or “cART”).

In certain embodiments, a compound of the present disclosure, or a pharmaceutically acceptable salt thereof for use in medical therapy of an HIV viral infection (e.g. HIV-1 or the replication of the HIV virus (e.g. HIV-1) or AIDS or delaying the onset of AIDS in an individual (e.g., a human)) is provided.

In certain embodiments, a compound of the present disclosure, or a pharmaceutically acceptable salt thereof for use in the manufacture of a medicament for treating an HIV viral infection or the replication of the HIV virus or AIDS or delaying the onset of AIDS in an individual (e.g., a human). One embodiment provides a compound of the present disclosure, or a pharmaceutically acceptable salt thereof, for use in the prophylactic or therapeutic treatment of an HIV infection or AIDS or for use in the therapeutic treatment or delaying the onset of AIDS is provided.

In certain embodiments, the use of a compound of the present disclosure (e.g. a compound of Formula (I)), or a pharmaceutically acceptable salt thereof, for the manufacture of a medicament for an HIV virus infection in an individual (e.g., a human) is provided. In certain embodiments, a compound of the present disclosure (e.g. a compound of Formula (I)), or a pharmaceutically acceptable salt thereof, for use in the prophylactic or therapeutic treatment of an HIV virus infection is provided.

In certain embodiments, in the methods of use, the administration is to an individual (e.g., a human) in need of the treatment. In certain embodiments, in the methods of use, the administration is to an individual (e.g., a human) who is at risk of developing AIDS.

Provided herein is a compound of the present disclosure (e.g. a compound of Formula (I)), or a pharmaceutically acceptable salt thereof, for use in therapy. In one embodiment, the compound of the present disclosure, or a pharmaceutically acceptable salt thereof, is for use in a method of treating an HIV viral infection or the replication of the HIV virus or AIDS or delaying the onset of AIDS in an individual (e.g., a human).

Also provided herein is a compound of the present disclosure (e.g. a compound of Formula (I)), or a pharmaceutically acceptable salt thereof, for use in a method of treating or preventing HIV in an individual in need thereof. In certain embodiments, the individual in need thereof is a human who has been infected with HIV. In certain embodiments, the individual in need thereof is a human who has been infected with HIV but who has not developed AIDS. In certain embodiments, the individual in need thereof is an individual at risk for developing AIDS. In certain embodiments, the individual in need thereof is a human who has been infected with HIV and who has developed AIDS.

Also provided herein is a compound of the present disclosure (e.g. a compound of Formula (I)), or a pharmaceutically acceptable salt thereof, for use in the therapeutic treatment or delaying the onset of AIDS.

Also provided herein is a compound of the present disclosure (e.g. a compound of Formula (I)), or a pharmaceutically acceptable salt thereof, for use in the prophylactic or therapeutic treatment of an HIV infection.

In certain embodiments, the HIV infection is an HIV-1 infection.

Additionally, the compounds of this disclosure are useful in the treatment of cancer or tumors (including dysplasias, such as uterine dysplasia). These includes hematological malignancies, oral carcinomas (for example of the lip, tongue or pharynx), digestive organs (for example esophagus, stomach, small intestine, colon, large intestine, or rectum), peritoneum, liver and biliary passages, pancreas, respiratory system such as larynx or lung (small cell and non-small cell), bone, connective tissue, skin (e.g., melanoma), breast, reproductive organs (fallopian tube, uterus, cervix, testicles, ovary, or prostate), urinary tract (e.g., bladder or kidney), brain and endocrine glands such as the thyroid. In summary, the compounds of this disclosure are employed to treat any neoplasm, including not only hematologic malignancies but also solid tumors of all kinds. In certain embodiments, the compounds are useful for treating a form of cancer selected from ovarian cancer, breast cancer, head and neck cancer, renal cancer, bladder cancer, hepatocellular cancer, and colorectal cancer.

Hematological malignancies are broadly defined as proliferative disorders of blood cells and/or their progenitors, in which these cells proliferate in an uncontrolled manner. Anatomically, the hematologic malignancies are divided into two primary groups: lymphomas—malignant masses of lymphoid cells, primarily but not exclusively in lymph nodes, and leukemias—neoplasm derived typically from lymphoid or myeloid cells and primarily affecting the bone marrow and peripheral blood. The lymphomas can be sub-divided into Hodgkin's Disease and Non-Hodgkin's lymphoma (NHL). The later group comprises several distinct entities, which can be distinguished clinically (e.g. aggressive lymphoma, indolent lymphoma), histologically (e.g. follicular lymphoma, mantle cell lymphoma) or based on the origin of the malignant cell (e.g. B lymphocyte, T lymphocyte). Leukemias and related malignancies include acute myelogenous leukemia (AML), chronic myelogenous leukemia (CML), acute lymphoblastic leukemia (ALL) and chronic lymphocytic leukemia (CLL). Other hematological malignancies include the plasma cell dyscrasias including multiple myeloma, and the myelodysplastic syndromes.

In certain embodiments, the compounds of the present disclosure are useful in the treatment of B-cell lymphoma, lymphoplasmacytoid lymphoma, fallopian tube cancer, head and neck cancer, ovarian cancer, and peritoneal cancer.

In certain embodiments, the compounds of the present disclosure are useful in the treatment of hepatocellular carcinoma, gastric cancer, and/or colorectal cancer. In certain embodiments, the compounds of the present disclosure are useful in the treatment of prostate cancer, breast cancer, and/or ovarian cancer. In certain embodiments, the compounds of the present disclosure are useful in the treatment of recurrent or metastatic squamous cell carcinoma.

In certain embodiments, a method of treating a hyperproliferative disease, comprising administering to an individual (e.g. a human) in need thereof a therapeutically effective amount of a compound of the present disclosure, or a pharmaceutically acceptable salt thereof, is provided. In certain embodiments, the hyperproliferative disease is cancer. In certain embodiments, the cancer is a solid tumor. In certain embodiments, the cancer is selected from ovarian cancer, breast cancer, head and neck cancer, renal cancer, bladder cancer, hepatocellular cancer, and colorectal cancer. In certain embodiments, the cancer is a lymphoma. In certain embodiments, the cancer is Hodgkin's lymphoma. In certain embodiments, the cancer is non-Hodgkin's lymphoma. In certain embodiments, the cancer is B-cell lymphoma. In certain embodiments, the cancer is selected from B-cell lymphoma; fallopian tube cancer, head and neck cancer, ovarian cancer and peritoneal cancer. In certain embodiments, the method further comprises administering one or more additional therapeutic agents as more fully described herein.

In certain embodiments, the cancer is prostate cancer, breast cancer, ovarian cancer, hepatocellular carcinoma, gastric cancer, colorectal cancer and/or recurrent or metastatic squamous cell carcinoma. In certain embodiments, the cancer is prostate cancer, breast cancer, and/or ovarian cancer. In certain embodiments, the cancer is hepatocellular carcinoma, gastric cancer, and/or colorectal cancer. In certain embodiments, the cancer is recurrent or metastatic squamous cell carcinoma.

### V. Administration

In some embodiments, in the methods of use, the administration is to an individual (e.g., a human) in need of the treatment.

Additional examples of diseases, disorders, or conditions include psoriasis, systemic lupus erythematosus and allergic rhinitis

In one embodiment, the compound of the present disclosure, or a pharmaceutically acceptable salt thereof, is for use in a method of treating a hyperproliferative disease (e.g. cancer) in an individual (e.g., a human).

Also provided herein is the use of a compound of the present disclosure (e.g. a compound of Formula (I)) or a pharmaceutically acceptable salt thereof for the manufacture of a medicament for treating a hyperproliferative disease (e.g. cancer) is provided.

### VI. Administration

One or more of the compounds of the present disclosure (also referred to herein as the active ingredients), can be administered by any route appropriate to the condition to be treated. Suitable routes include oral, rectal, nasal, topical (including buccal and sublingual), transdermal, vaginal and parenteral (including subcutaneous, intramuscular, intravenous, intradermal, intrathecal and epidural), and the like. It will be appreciated that the preferred route may vary with for example the condition of the recipient. An advantage of certain compounds disclosed herein is that they are orally bioavailable and can be dosed orally.

A compound of the present disclosure, such as a compound of Formula (I), may be administered to an individual in accordance with an effective dosing regimen for a desired period of time or duration, such as at least about one month, at least about 2 months, at least about 3 months, at least about 6 months, or at least about 12 months or longer. In one variation, the compound is administered on a daily or intermittent schedule for the duration of the individual's life.

The dosage or dosing frequency of a compound of the present disclosure may be adjusted over the course of the treatment, based on the judgment of the administering physician.

The compound may be administered to an individual (e.g., a human) in an effective amount. In certain embodiments, the compound is administered once daily.

In certain embodiments, methods for treating or preventing a disease or condition in a human are provided, comprising administering to the human a

therapeutically effective amount of a compound of the present disclosure, or a pharmaceutically acceptable salt thereof, in combination with a therapeutically effective amount of one or more (e.g., one, two, three, four, one or two, one to three, or one to four) additional therapeutic agents. As modulators of TLR-8 may be used in the treatment of various diseases or conditions, the particular identity of the additional therapeutic agents will depend on the particular disease or condition being treated.

The compound of Formula (J), (I), (II), (IIa), (IIb), (III), (IIIa), (IIIb), (IV), (IVa), (IVb), (IVc), or (IVd) can be administered by any useful route and means, such as by oral or parenteral (e.g., intravenous) administration. Therapeutically effective amounts of the compound of Formula (J), (I), (II), (IIa), (IIb), (III), (IIIa), (IIIb), (IV), (IVa), (IVb), (IVc), or (IVd) are from about 0.00001 mg/kg body weight per day to about 10 mg/kg body weight per day, such as from about 0.0001 mg/kg body weight per day to about 10 mg/kg body weight per day, or such as from about 0.001 mg/kg body weight per day to about 1 mg/kg body weight per day, or such as from about 0.01 mg/kg body weight per day to about 1 mg/kg body weight per day, or such as from about 0.05 mg/kg body weight per day to about 0.5 mg/kg body weight per day, or such as from about 0.3 μg to about 30 mg per day, or such as from about 30 μg to about 300 μg per day.

A compound of the present disclosure (e.g., any compound of Formula (I)) may be combined with one or more additional therapeutic agents in any dosage amount of the compound of the present disclosure (e.g., from 1 mg to 1000 mg of compound). Therapeutically effective amounts of the compound of Formula (J), (I), (II), (IIa), (IIb), (III), (IIIa), (IIIb), (IV), (IVa), (IVb), (IVc), or (IVd), are from about 0.01 mg per dose to about 1000 mg per dose, such as from about 0.01 mg per dose to about 100 mg per dose, or such as from about 0.1 mg per dose to about 100 mg per dose, or such as from about 1 mg per dose to about 100 mg per dose, or such as from about 1 mg per dose to about 10 mg per dose. Other therapeutically effective amounts of the compound of Formula (J), (I), (II), (IIa), (IIb), (III), (IIIa), (IIIb), (IV), (IVa), (IVb), (IVc), or (IVd) are about 1 mg per dose, or about 2, 3, 4, 5, 6, 7, 8, 9, 10, 15, 20, 25, 30, 35, 40, 45, 50, 55, 60, 65, 70, 75, 80, 85, 90, 95, or about 100 mg per dose. Other therapeutically effective amounts of the compound of Formula (J), (I), (II), (IIa), (IIb), (III), (IIIa), (IIIb), (IV), (IVa), (IVb), (IVc), or (IVd) are about 100 mg per dose, or about 125, 150, 175, 200, 225, 250, 275, 300, 350, 400, 450, or about 500 mg per dose. A single dose can be administered hourly, daily, or weekly. For example, a single dose can be administered once every 1 hour, 2, 3, 4, 6, 8, 12, 16 or once every 24 hours. A single dose can also be administered once every 1 day, 2, 3, 4, 5, 6, or once every 7 days. A single dose can also be administered once every 1 week, 2, 3, or once every 4 weeks. In certain embodiments, a single dose can be administered once every week. A single dose can also be administered once every month.

The frequency of dosage of the compound of Formula (J), (I), (II), (IIa), (IIb), (III), (IIIa), (IIIb), (IV), (IVa), (IVb), (IVc), or (IVd) will be determined by the needs of the individual patient and can be, for example, once per day or twice, or more times, per day. Administration of the compound continues for as long as necessary to treat the HBV or HCV infection. For example, Compound I can be administered to a human being infected with HBV or HCV for a period of from 20 days to 180 days or, for example, for a period of from 20 days to 90 days or, for example, for a period of from 30 days to 60 days.

Administration can be intermittent, with a period of several or more days during which a patient receives a daily dose of the compound of Formula (J), (I), (II), (IIa), (IIb), (III), (IIIa), (IIIb), (IV), (IVa), (IVb), (IVc), or (IVd), followed by a period of several or more days during which a patient does not receive a daily dose of the compound. For example, a patient can receive a dose of the compound every other day, or three times per week. Again by way of example, a patient can receive a dose of the compound each day for a period of from 1 to 14 days, followed by a period of 7 to 21 days during which the patient does not receive a dose of the compound, followed by a subsequent period (e.g., from 1 to 14 days) during which the patient again receives a daily dose of the compound. Alternating periods of administration of the compound, followed by non-administration of the compound, can be repeated as clinically required to treat the patient.

In one embodiment, pharmaceutical compositions comprising a compound of the present disclosure, or a pharmaceutically acceptable salt thereof, in combination with one or more (e.g., one, two, three, four, one or two, one to three, or one to four) additional therapeutic agents, and a pharmaceutically acceptable excipient are provided.

In one embodiment, kits comprising a compound of the present disclosure, or a pharmaceutically acceptable salt thereof, in combination with one or more (e.g., one, two, three, four, one or two, one to three, or one to four) additional therapeutic agents are provided.

In certain embodiments, a compound of the present disclosure, or a pharmaceutically acceptable salt thereof, is combined with one, two, three, four or more additional therapeutic agents. In certain embodiments, a compound of the present disclosure, or a pharmaceutically acceptable salt thereof, is combined with two additional therapeutic agents. In other embodiments, a compound of the present disclosure, or a pharmaceutically acceptable salt thereof, is combined with three additional therapeutic agents. In further embodiments, a compound of the present disclosure, or a pharmaceutically acceptable salt thereof, is combined with four additional therapeutic agents. The one, two, three, four or more additional therapeutic agents can be different therapeutic agents selected from the same class of therapeutic agents, and/or they can be selected from different classes of therapeutic agents.

In certain embodiments, when a compound of the present disclosure is combined with one or more additional therapeutic agents as described herein, the components of the composition are administered as a simultaneous or sequential regimen. When administered sequentially, the combination may be administered in two or more administrations.

In certain embodiments, a compound of the present disclosure is combined with one or more additional therapeutic agents in a unitary dosage form for simultaneous administration to a patient, for example as a solid dosage form for oral administration.

In certain embodiments, a compound of the present disclosure is administered with one or more additional therapeutic agents. Co-administration of a compound of the present disclosure with one or more additional therapeutic agents generally refers to simultaneous or sequential administration of a compound of the present disclosure and one or more additional therapeutic agents, such that therapeutically effective amounts of the compound disclosed herein and one or more additional therapeutic agents are both present in the body of the patient.

Co-administration includes administration of unit dosages of the compounds disclosed herein before or after administration of unit dosages of one or more additional therapeutic agents, for example, administration of the compound disclosed herein within seconds, minutes, or hours of the administration of one or more additional therapeutic agents. For example, in some embodiments, a unit dose of a compound of the present disclosure is administered first, followed within seconds or minutes by administration of a unit dose of one or more additional therapeutic agents. Alternatively, in other embodiments, a unit dose of one or more additional therapeutic agents is administered first, followed by administration of a unit dose of a compound of the present disclosure within seconds or minutes. In some embodiments, a unit dose of a compound of the present disclosure is administered first, followed, after a period of hours (e.g., 1-12 hours), by administration of a unit dose of one or more additional therapeutic agents. In other embodiments, a unit dose of one or more additional therapeutic agents is administered first, followed, after a period of hours (e.g., 1-12 hours), by administration of a unit dose of a compound of the present disclosure.

### VII. Combination Therapy for HBV

In certain embodiments, a method for treating or preventing an HBV infection in a human having or at risk of having the infection is provided, comprising administering to the human a therapeutically effective amount of a compound of the present disclosure, or a pharmaceutically acceptable salt thereof, in combination with a therapeutically effective amount of one or more (e.g., one, two, three, four, one or two, one to three or one to four) additional therapeutic agents. In one embodiment, a method for treating an HBV infection in a human having or at risk of having the infection is provided, comprising administering to the human a therapeutically effective amount of a compound of the present disclosure, or a pharmaceutically acceptable salt thereof, in combination with a therapeutically effective amount of one or more (e.g., one, two, three, four, one or two, one to three or one to four) additional therapeutic agents.

In certain embodiments, the present disclosure provides a method for treating an HBV infection, comprising administering to a patient in need thereof a therapeutically effective amount of a compound of the present disclosure, or a pharmaceutically acceptable salt thereof, in combination with a therapeutically effective amount of one or more additional therapeutic agents which are suitable for treating an HBV infection. In certain embodiments, one or more additional therapeutic agents includes, for example, one, two, three, four, one or two, one to three or one to four additional therapeutic agents.

In the above embodiments, the additional therapeutic agent may be an anti-HBV agent. For example, in some embodiments, the additional therapeutic agent is selected from the group consisting of HBV combination drugs, HBV DNA polymerase inhibitors, immunomodulators, toll-like receptor modulators (modulators of TLR-1, TLR-2, TLR-3, TLR-4, TLR-5, TLR-6, TLR-7, TLR-8, TLR-9, TLR-10, TLR-11, TLR-12 and TLR-13), interferon alpha receptor ligands, hyaluronidase inhibitors, recombinant IL-7, hepatitis B surface antigen (HBsAg) inhibitors, compounds targeting hepatitis B core antigen (HbcAg), cyclophilin inhibitors, HBV therapeutic vaccines, HBV prophylactic vaccines, HBV viral entry inhibitors, NTCP (Na+-taurocholate cotransporting polypeptide) inhibitors, antisense oligonucleotide targeting viral mRNA, short interfering RNAs (siRNA), miRNA gene therapy agents, endonuclease modulators, inhibitors of ribonucleotide reductase, hepatitis B virus E antigen inhibitors, recombinant scavenger receptor A (SRA) proteins, Src kinase inhibitors, HBx inhibitors, cccDNA inhibitors, short synthetic hairpin RNAs (sshRNAs), HBV antibodies including HBV antibodies targeting the surface antigens of the hepatitis B virus and bispecific antibodies and “antibody-like” therapeutic proteins (such as DARTs®, Duobodies®, Bites®, XmAbs®, TandAbs®, Fab derivatives), CCR2 chemokine antagonists, thymosin agonists, cytokines, nucleoprotein inhibitors (HBV core or capsid protein inhibitors), stimulators of retinoic acid-inducible gene 1, stimulators of NOD2, stimulators of NOD1, Arginase-1 inhibitors, STING agonists, PI3K inhibitors, lymphotoxin beta receptor activators, Natural Killer Cell Receptor 2B4 inhibitors, Lymphocyte-activation gene 3 inhibitors, CD160 inhibitors, cytotoxic T-lymphocyte-associated protein 4 inhibitors, CD137 inhibitors, Killer cell lectin-like receptor subfamily G member 1 inhibitors, TIM-3 inhibitors, B- and T-lymphocyte attenuator inhibitors, CD305 inhibitors, PD-1 inhibitors, PD-L1 inhibitors, PEG-Interferon Lambda, recombinant thymosin alpha-1, BTK inhibitors, modulators of TIGIT, modulators of CD47, modulators of SIRPalpha, modulators of ICOS, modulators of CD27, modulators of CD70, modulators of OX40, modulators of NKG2D, modulators of Tim-4, modulators of B7-H4, modulators of B7-H3, modulators of NKG2A, modulators of GITR, modulators of CD160, modulators of HEVEM, modulators of CD161, modulators of Axl, modulators of Mer, modulators of Tyro, gene modifiers or editors such as CRISPR (including CRISPR Cas9), zinc finger nucleases or synthetic nucleases (TALENs), Hepatitis B virus replication inhibitors, compounds such as those disclosed in U.S. Publication No. 2010/0143301 (Gilead Sciences), U.S. Publication No. 2011/0098248 (Gilead Sciences), U.S. Publication No. 2009/0047249 (Gilead Sciences), U.S. Pat. No. 8,722,054 (Gilead Sciences), U.S. Publication No. 2014/0045849 (Janssen), U.S. Publication No. 2014/0073642 (Janssen), WO2014/056953 (Janssen), WO2014/076221 (Janssen), WO2014/128189 (Janssen), U.S. Publication No. 2014/0350031 (Janssen), WO2014/023813 (Janssen), U.S. Publication No. 2008/0234251 (Array Biopharma), U.S. Publication No. 2008/0306050 (Array Biopharma), U.S. Publication No. 2010/0029585 (Ventirx Pharma), U.S. Publication No. 2011/0092485 (Ventirx Pharma), US2011/0118235 (Ventirx Pharma), U.S. Publication No. 2012/0082658 (Ventirx Pharma), U.S. Publication No. 2012/0219615 (Ventirx Pharma), U.S. Publication No. 2014/0066432 (Ventirx Pharma), U.S. Publication No. 2014/0088085 (Ventirx Pharma), U.S. Publication No. 2014/0275167 (Novira Therapeutics), U.S. Publication No. 2013/0251673 (Novira Therapeutics), U.S. Pat. No. 8,513,184 (Gilead Sciences), U.S. Publication No. 2014/0030221 (Gilead Sciences), U.S. Publication No. 2013/0344030 (Gilead Sciences), U.S. Publication No. 2013/0344029 (Gilead Sciences), U.S. Publication No. 2014/0343032 (Roche), WO2014037480 (Roche), U.S. Publication No. 2013/0267517 (Roche), WO2014131847 (Janssen), WO2014033176 (Janssen), WO2014033170 (Janssen), WO2014033167 (Janssen), U.S. Publication No. 2014/0330015 (Ono Pharmaceutical), U.S. Publication No. 2013/0079327 (Ono Pharmaceutical), U.S. Publication No. 2013/0217880 (Ono pharmaceutical), and other drugs for treating HBV, and combinations thereof. In some embodiments, the additional therapeutic agent is further selected from hepatitis B surface antigen (HBsAg) secretion or assembly inhibitors, TCR-like antibodies, IDO inhibitors, cccDNA epigenetic modifiers, IAPs inhibitors, SMAC mimetics, and compounds such as those disclosed in US20100015178 (Incyte).

In certain embodiments, the additional therapeutic is selected from the group consisting of HBV combination drugs, HBV DNA polymerase inhibitors, toll-like receptor 7 modulators, toll-like receptor 8 modulators, Toll-like receptor 7 and 8 modulators, Toll-like receptor 3 modulators, interferon alpha receptor ligands, HBsAg inhibitors, compounds targeting HbcAg, cyclophilin inhibitors, HBV therapeutic vaccines, HBV prophylactic vaccines, HBV viral entry inhibitors, NTCP inhibitors, antisense oligonucleotide targeting viral mRNA, short interfering RNAs (siRNA), hepatitis B virus E antigen inhibitors, HBx inhibitors, cccDNA inhibitors, HBV antibodies including HBV antibodies targeting the surface antigens of the hepatitis B virus, thymosin agonists, cytokines, nucleoprotein inhibitors (HBV core or capsid protein inhibitors), stimulators of retinoic acid-inducible gene 1, stimulators of NOD2, stimulators of NOD1, recombinant thymosin alpha-1, BTK inhibitors, and hepatitis B virus replication inhibitors, and combinations thereof. In certain embodiments, the additional therapeutic is selected from hepatitis B surface antigen (HBsAg) secretion or assembly inhibitors and IDO inhibitors.

In certain embodiments a compound of the present disclosure (e.g a compound of Formula (I)) is formulated as a tablet, which may optionally contain one or more other compounds useful for treating HBV. In certain embodiments, the tablet can contain another active ingredient for treating HBV, such as HBV DNA polymerase inhibitors, immunomodulators, toll-like receptor modulators (modulators of TLR-1, TLR-2, TLR-3, TLR-4, TLR-5, TLR-6, TLR-7, TLR-8, TLR-9, TLR-10, TLR-11, TLR-12 and TLR-13), modulators of tlr7, modulators of tlr8, modulators of tlr7 and tlr8, interferon alpha receptor ligands, hyaluronidase inhibitors, hepatitis B surface antigen (HBsAg) inhibitors, compounds targeting hepatitis B core antigen (HbcAg), cyclophilin inhibitors, HBV viral entry inhibitors, NTCP (Na+-taurocholate cotransporting polypeptide) inhibitors, endonuclease modulators, inhibitors of ribonucleotide reductase, hepatitis B virus E antigen inhibitors, Src kinase inhibitors, HBx inhibitors, cccDNA inhibitors, CCR2 chemokine antagonists, thymosin agonists, nucleoprotein inhibitors (HBV core or capsid protein inhibitors), stimulators of retinoic acid-inducible gene 1, stimulators of NOD2, stimulators of NOD1, Arginase-1 inhibitors, STING agonists, PI3K inhibitors, lymphotoxin beta receptor activators, Natural Killer Cell Receptor 2B4 inhibitors, Lymphocyte-activation gene 3 inhibitors, CD160 inhibitors, cytotoxic T-lymphocyte-associated protein 4 inhibitors, CD137 inhibitors, Killer cell lectin-like receptor subfamily G member 1 inhibitors, TIM-3 inhibitors, B- and T-lymphocyte attenuator inhibitors, CD305 inhibitors, PD-1 inhibitors, PD-L1 inhibitors, BTK inhibitors, modulators of TIGIT, modulators of CD47, modulators of SIRP alpha, modulators of ICOS, modulators of CD27, modulators of CD70, modulators of OX40, modulators of NKG2D, modulators of Tim-4, modulators of B7-H4, modulators of B7-H3, modulators of NKG2A, modulators of GITR, modulators of CD160, modulators of HEVEM, modulators of CD161, modulators of Axl, modulators of Mer, modulators of Tyro, and Hepatitis B virus replication inhibitors, and combinations thereof. In certain embodiments, the tablet can contain another active ingredient for treating HBV, such as hepatitis B surface antigen (HBsAg) secretion or assembly inhibitors, cccDNA epigenetic modifiers, IAPs inhibitors, SMAC mimetics, and IDO inhibitors.

In certain embodiments, such tablets are suitable for once daily dosing.

In certain embodiments, the additional therapeutic agent is selected from one or more of:

(1) Combination drugs selected from the group consisting of tenofovir disoproxil fumarate+emtricitabine (TRUVADA®); adefovir+clevudine and GBV-015, as well as combination drugs selected from ABX-203+lamivudine+PEG-IFNalpha, ABX-203+adefovir+PEG-IFNalpha, and INO-9112+RG7944 (INO-1800);
(2) HBV DNA polymerase inhibitors selected from the group consisting of besifovir, entecavir (Baraclude®), adefovir (Hepsera®), tenofovir disoproxil fumarate (Viread®), tenofovir alafenamide, tenofovir, tenofovir disoproxil, tenofovir alafenamide fumarate, tenofovir alafenamide hemifumarate, tenofovir dipivoxil, tenofovir dipivoxil fumarate, tenofovir octadecyloxyethyl ester, telbivudine (Tyzeka®), pradefovir, Clevudine, emtricitabine (Emtriva®), ribavirin, lamivudine (Epivir-HBV®), phosphazide, famciclovir, SNC-019754, FMCA, fusolin, AGX-1009 and metacavir, as well as HBV DNA polymerase inhibitors selected from AR-II-04-26 and HS-10234;
(3) Immunomodulators selected from the group consisting of rintatolimod, imidol hydrochloride, ingaron, dermaVir, plaquenil (hydroxychloroquine), proleukin, hydroxyurea, mycophenolate mofetil (MPA) and its ester derivative mycophenolate mofetil (MMF), WF-10, ribavirin, IL-12, polymer polyethyleneimine (PEI), Gepon, VGV-1, MOR-22, BMS-936559 and IR-103, as well as immunomodulators selected from INO-9112, polymer polyethyleneimine (PEI), Gepon, VGV-1, MOR-22, BMS-936559, RO-7011785, RO-6871765 and IR-103;
(4) Toll-like receptor 7 modulators selected from the group consisting of GS-9620, GSK-2245035, imiquimod, resiquimod, DSR-6434, DSP-3025, IMO-4200, MCT-465, 3M-051, SB-9922, 3M-052, Limtop, TMX-30X, TMX-202 RG-7863 and RG-7795;
(5) Toll-like receptor 8 modulators selected from the group consisting of motolimod, resiquimod, 3M-051, 3M-052, MCT-465, IMO-4200, VTX-763, VTX-1463;
(6) Toll-like receptor 3 modulators selected from the group consisting of rintatolimod, poly-ICLC, MCT-465, MCT-475, Riboxxon, Riboxxim and ND-1.1;
(7) Interferon alpha receptor ligands selected from the group consisting of interferon alpha-2b (Intron A®), pegylated interferon alpha-2a (Pegasys®), interferon alpha 1b (Hapgen®), Veldona, Infradure, Roferon-A, YPEG-interferon alfa-2a (YPEG-rhIFNalpha-2a), P-1101, Algeron, Alfarona, Ingaron (interferon gamma), rSIFN-co (recombinant super compound interferon), Ypeginterferon alfa-2b (YPEG-rhIFNalpha-2b), MOR-22, peginterferon alfa-2b (PEG-Intron®), Bioferon, Novaferon, Inmutag (Inferon), Multiferon®, interferon alfa-n1 (Humoferon®), interferon beta-1a (Avonex®), Shaferon, interferon alfa-2b (AXXO), Alfaferone, interferon alfa-2b (BioGeneric Pharma), interferon-alpha 2 (CJ), Laferonum, VIPEG, BLAUFERON-B, BLAUFERON-A, Intermax Alpha, Realdiron, Lanstion, Pegaferon, PDferon-B PDferon-B, interferon alfa-2b (IFN, Laboratorios Bioprofarma), alfainterferona 2b, Kalferon, Pegnano, Feronsure, PegiHep, interferon alfa 2b (Zydus-Cadila), Optipeg A, Realfa 2B, Reliferon, interferon alfa-2b (Amega), interferon alfa-2b (Virchow), peginterferon alfa-2b (Amega), Reaferon-EC, Proquiferon, Uniferon, Urifron, interferon alfa-2b (Changchun Institute of Biological Products), Anterferon, Shanferon, Layfferon, Shang Sheng Lei Tai, INTEFEN, SINOGEN, Fukangtai, Pegstat, rHSA-IFN alpha-2b and Interapo (Interapa);
(8) Hyaluronidase inhibitors selected from the group consisting of astodrimer;

### (9) Modulators of IL-10;

(10) HBsAg inhibitors selected from the group consisting of HBF-0259, PBHBV-001, PBHBV-2-15, PBHBV-2-1, REP 9AC, REP-9C and REP 9AC′, as well as HBsAg inhibitors selected from REP-9, REP-2139, REP-2139-Ca, REP-2165, REP-2055, REP-2163, REP-2165, REP-2053, REP-2031 and REP-006 and REP-9AC′ (11) Toll like receptor 9 modulators selected from CYT003, as well as Toll like receptor 9 modulators selected from CYT-003, IMO-2055, IMO-2125, IMO-3100, IMO-8400, IMO-9200, agatolimod, DIMS-9054, DV-1179, AZD-1419, MGN-1703, and CYT-003-QbG10;
(12) Cyclophilin inhibitors selected from the group consisting of OCB-030, SCY-635 and NVP-018;
(13) HBV Prophylactic vaccines selected from the group consisting of Hexaxim, Heplisav, Mosquirix, DTwP-HBV vaccine, Bio-Hep-B, D/T/P/HBV/M (LBVP-0101; LBVW-0101), DTwP-Hepb-Hib-IPV vaccine, Heberpenta L, DTwP-HepB-Hib, V-419, CVI-HBV-001, Tetrabhay, hepatitis B prophylactic vaccine (Advax Super D), Hepatrol-07, GSK-223192A, Engerix B®, recombinant hepatitis B vaccine (intramuscular, Kangtai Biological Products), recombinant hepatitis B vaccine (Hansenual polymorpha yeast, intramuscular, Hualan Biological Engineering), Bimmugen, Euforavac, Eutravac, anrix-DTaP-IPV-Hep B, Infanrix-DTaP-IPV-Hep B-Hib, Pentabio Vaksin DTP-HB-Hib, Comvac 4, Twinrix, Euvax-B, Tritanrix HB, Infanrix Hep B, Comvax, DTP-Hib-HBV vaccine, DTP-HBV vaccine, Yi Tai, Heberbiovac HB, Trivac HB, GerVax, DTwP-Hep B-Hib vaccine, Bilive, Hepavax-Gene, SUPERVAX, Comvac5, Shanvac-B, Hebsulin, Recombivax HB, Revac B mcf, Revac B+, Fendrix, DTwP-HepB-Hib, DNA-001, Shan6, rhHBsAG vaccine, and DTaP-rHB-Hib vaccine;
(14) HBV Therapeutic vaccines selected from the group consisting of HBsAG-HBIG complex, Bio-Hep-B, NASVAC, abi-HB (intravenous), ABX-203, Tetrabhay, GX-110E, GS-4774, peptide vaccine (epsilonPA-44), Hepatrol-07, NASVAC (NASTERAP), IMP-321, BEVAC, Revac B mcf, Revac B+, MGN-1333, KW-2, CVI-HBV-002, AltraHepB, VGX-6200, FP-02, TG-1050, NU-500, HBVax, im/TriGrid/antigen vaccine, Mega-CD40L-adjuvanted vaccine, HepB-v, NO-1800, recombinant VLP-based therapeutic vaccine (HBV infection, VLP Biotech), AdTG-17909, AdTG-17910 AdTG-18202, ChronVac-B, and Lm HBV, as well as HBV Therapeutic vaccines selected from FP-02.2 and RG7944 (INO-1800);
(15) HBV viral entry inhibitor selected from the group consisting of Myrcludex B;
(16) Antisense oligonucleotide targeting viral mRNA selected from the group consisting of ISIS-HBVRx;
(17) short interfering RNAs (siRNA) selected from the group consisting of TKM-HBV (TKM-HepB), ALN-HBV, SR-008, ddRNAi and ARC-520;
(18) Endonuclease modulators selected from the group consisting of PGN-514;
(19) Inhibitors of ribonucleotide reductase selected from the group consisting of Trimidox;
(20) Hepatitis B virus E antigen inhibitors selected from the group consisting of wogonin;
(21) HBV antibodies targeting the surface antigens of the hepatitis B virus selected from the group consisting of GC-1102, XTL-17, XTL-19, XTL-001, KN-003 and fully human monoclonal antibody therapy (hepatitis B virus infection, Humabs BioMed), as well as HBV antibodies targeting the surface antigens of the hepatitis B virus selected from IV Hepabulin SN;
(22) HBV antibodies including monoclonal antibodies and polyclonal antibodies selected from the group consisting of Zutectra, Shang Sheng Gan Di, Uman Big (Hepatitis B Hyperimmune), Omri-Hep-B, Nabi-HB, Hepatect CP, HepaGam B, igantibe, Niuliva, CT-P24, hepatitis B immunoglobulin (intravenous, pH4, HBV infection, Shanghai RAAS Blood Products) and Fovepta (BT-088);
(23) CCR2 chemokine antagonists selected from the group consisting of propagermanium;
(24) Thymosin agonists selected from the group consisting of Thymalfasin;
(25) Cytokines selected from the group consisting of recombinant IL-7, CYT-107, interleukin-2 (IL-2, Immunex); recombinant human interleukin-2 (Shenzhen Neptunus) and celmoleukin, as well as cytokines selected from IL-15, IL-21, IL-24; (26) Nucleoprotein inhibitors (HBV core or capsid protein inhibitors) selected from the group consisting of NVR-1221, NVR-3778, BAY 41-4109, morphothiadine mesilate and DVR-23;
(27) Stimulators of retinoic acid-inducible gene 1 selected from the group consisting of SB-9200, SB-40, SB-44, ORI-7246, ORI-9350, ORI-7537, ORI-9020, ORI-9198 and ORI-7170;
(28) Stimulators of NOD2 selected from the group consisting of SB-9200;
(29) Recombinant thymosin alpha-1 selected from the group consisting of NL-004 and PEGylated thymosin alpha 1;
(30) Hepatitis B virus replication inhibitors selected from the group consisting of isothiafludine, IQP-HBV, RM-5038 and Xingantie;
(31) PI3K inhibitors selected from the group consisting of idelalisib, AZD-8186, buparlisib, CLR-457, pictilisib, neratinib, rigosertib, rigosertib sodium, EN-3342, TGR-1202, alpelisib, duvelisib, UCB-5857, taselisib, XL-765, gedatolisib, VS-5584, copanlisib, CAI orotate, perifosine, RG-7666, GSK-2636771, DS-7423, panulisib, GSK-2269557, GSK-2126458, CUDC-907, PQR-309, INCB-040093, pilaralisib, BAY-1082439, puquitinib mesylate, SAR-245409, AMG-319, RP-6530, ZSTK-474, MLN-1117, SF-1126, RV-1729, sonolisib, LY-3023414, SAR-260301 and CLR-1401;
(32) cccDNA inhibitors selected from the group consisting of BSBI-25;
(33) PD-L1 inhibitors selected from the group consisting of MEDI-0680, RG-7446, durvalumab, KY-1003, KD-033, MSB-0010718C, TSR-042, ALN-PDL, STI-A1014 and BMS-936559;
(34) PD-1 inhibitors selected from the group consisting of nivolumab, pembrolizumab, pidilizumab, BGB-108 and mDX-400;
(35) BTK inhibitors selected from the group consisting of ACP-196, dasatinib, ibrutinib, PRN-1008, SNS-062, ONO-4059, BGB-3111, MSC-2364447, X-022, spebrutinib, TP-4207, HM-71224, KBP-7536, AC-0025;
(36) Other drugs for treating HBV selected from the group consisting of gentiopicrin (gentiopicroside), nitazoxanide, birinapant, NOV-205 (Molixan; BAM-205), Oligotide, Mivotilate, Feron, levamisole, Ka Shu Ning, Alloferon, WS-007, Y-101 (Ti Fen Tai), rSIFN-co, PEG-IIFNm, KW-3, BP-Inter-014, oleanolic acid, HepB-nRNA, cTP-5 (rTP-5), HSK-II-2, HEISCO-106-1, HEISCO-106, Hepbama, IBPB-006IA, Hepuyinfen, DasKloster 0014-01, Jiangantai (Ganxikang), picroside, GA5 NM-HBV, DasKloster-0039, hepulantai, IMB-2613, TCM-800B and ZH-2N, as well as other drugs for treating HBV selected from reduced glutathione, and RO-6864018; and
(37) The compounds disclosed in US20100143301 (Gilead Sciences), US20110098248 (Gilead Sciences), US20090047249 (Gilead Sciences), U.S. Pat. No. 8,722,054 (Gilead Sciences), US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (VentirxPharma), US20140275167 (Novira therapeutics), US20130251673 (Novira therapeutics), U.S. Pat. No. 8,513,184 (Gilead Sciences), US20140030221 (Gilead Sciences), US20130344030 (Gilead Sciences), US20130344029 (Gilead Sciences), US20140343032 (Roche), WO2014037480 (Roche), US20130267517 (Roche), WO2014131847 (Janssen), WO2014033176 (Janssen), WO2014033170 (Janssen), WO2014033167 (Janssen), US20140330015 (Ono pharmaceutical), US20130079327 (Ono pharmaceutical), and US20130217880 (Ono pharmaceutical), and the compounds disclosed in US20100015178 (Incyte).

Also included in the list above are:

(38) IDO inhibitors selected from the group consisting of epacadostat (INCB24360), F-001287, resminostat (4SC-201), SN-35837, NLG-919, GDC-0919, and indoximod;
(39) Arginase inhibitors selected from CB-1158, C-201, and resminostat; and
(40) Cytotoxic T-lymphocyte-associated protein 4 (ipi4) inhibitors selected from ipilumimab, belatacept, PSI-001, PRS-010, tremelimumab, and JHL-1155.

In certain embodiments, a compound of the present disclosure, or a pharmaceutically acceptable salt thereof, is combined with one, two, three, four or more additional therapeutic agents. In certain embodiments, a compound of the present disclosure, or a pharmaceutically acceptable salt thereof, is combined with two additional therapeutic agents. In other embodiments, a compound of the present disclosure, or a pharmaceutically acceptable salt thereof, is combined with three additional therapeutic agents. In further embodiments, a compound of the present disclosure, or a pharmaceutically acceptable salt thereof, is combined with four additional therapeutic agents. The one, two, three, four or more additional therapeutic agents can be different therapeutic agents selected from the same class of therapeutic agents, and/or they can be selected from different classes of therapeutic agents.

In a specific embodiment, a compound of the present disclosure, or a pharmaceutically acceptable salt thereof, is combined with an HBV DNA polymerase inhibitor. In another specific embodiment, a compound of the present disclosure, or a pharmaceutically acceptable salt thereof, is combined with an HBV DNA polymerase inhibitor and at least one additional therapeutic agent selected from the group consisting of: immunomodulators, toll-like receptor modulators (modulators of TLR-1, TLR-2, TLR-3, TLR-4, TLR-5, TLR-6, TLR-7, TLR-8, TLR-9, TLR-10, TLR-11, TLR-12 and TLR-13), interferon alpha receptor ligands, hyaluronidase inhibitors, recombinant IL-7, HBsAg inhibitors, compounds targeting HbcAg, cyclophilin inhibitors, HBV therapeutic vaccines, HBV prophylactic vaccines HBV viral entry inhibitors, NTCP inhibitors, antisense oligonucleotide targeting viral mRNA, short interfering RNAs (siRNA), miRNA gene therapy agents, endonuclease modulators, inhibitors of ribonucleotide reductase, Hepatitis B virus E antigen inhibitors, recombinant scavenger receptor A (SRA) proteins, src kinase inhibitors, HBx inhibitors, cccDNA inhibitors, short synthetic hairpin RNAs (sshRNAs), HBV antibodies including HBV antibodies targeting the surface antigens of the hepatitis B virus and bispecific antibodies and “antibody-like” therapeutic proteins (such as DARTs®, Duobodies®, Bites®, XmAbs®, TandAbs®, Fab derivatives), CCR2 chemokine antagonists, thymosin agonists, cytokines, nucleoprotein inhibitors (HBV core or capsid protein inhibitors), stimulators of retinoic acid-inducible gene 1, stimulators of NOD2, stimulators of NOD1, Arginase-1 inhibitors, STING agonists, PI3K inhibitors, lymphotoxin beta receptor activators, Natural Killer Cell Receptor 2B4 inhibitors, Lymphocyte-activation gene 3 inhibitors, CD160 inhibitors, cytotoxic T-lymphocyte-associated protein 4 inhibitors, CD137 inhibitors, Killer cell lectin-like receptor subfamily G member 1 inhibitors, TIM-3 inhibitors, B- and T-lymphocyte attenuator inhibitors, CD305 inhibitors, PD-1 inhibitors, PD-L1 inhibitors, PEG-Interferon Lambda, recombinant thymosin alpha-1, BTK inhibitors, modulators of TIGIT, modulators of CD47, modulators of SIRPalpha, modulators of ICOS, modulators of CD27, modulators of CD70, modulators of OX40, modulators of NKG2D, modulators of Tim-4, modulators of B7-H4, modulators of B7-H3, modulators of NKG2A, modulators of GITR, modulators of CD160, modulators of HEVEM, modulators of CD161, modulators of Axl, modulators of Mer, modulators of Tyro, gene modifiers or editors such as CRISPR (including CRISPR Cas9), zinc finger nucleases or synthetic nucleases (TALENs), and Hepatitis B virus replication inhibitors. In certain embodiments the at least one additional therapeutic agent is further selected from hepatitis B surface antigen (HBsAg) secretion or assembly inhibitors, TCR-like antibodies, cccDNA epigenetic modifiers, IAPs inhibitors, SMAC mimetics, and IDO inhibitors.

In another specific embodiment, a compound of the present disclosure, or a pharmaceutically acceptable salt thereof, is combined with an HBV DNA polymerase inhibitor and at least one additional therapeutic agent selected from the group consisting of: HBV viral entry inhibitors, NTCP inhibitors, HBx inhibitors, cccDNA inhibitors, HBV antibodies targeting the surface antigens of the hepatitis B virus, short interfering RNAs (siRNA), miRNA gene therapy agents, short synthetic hairpin RNAs (sshRNAs), and nucleoprotein inhibitors (HBV core or capsid protein inhibitors).

In another specific embodiment, a compound of the present disclosure, or a pharmaceutically acceptable salt thereof, is combined with an HBV DNA polymerase inhibitor, one or two additional therapeutic agents selected from the group consisting of: immunomodulators, toll-like receptor modulators (modulators of TLR-1, TLR-2, TLR-3, TLR-4, TLR-5, TLR-6, TLR-7, TLR-8, TLR-9, TLR-10, TLR-11, TLR-12 and TLR-13), HBsAg inhibitors, HBV therapeutic vaccines, HBV antibodies including HBV antibodies targeting the surface antigens of the hepatitis B virus and bispecific antibodies and “antibody-like” therapeutic proteins (such as DARTs®, Duobodies®, Bites®, XmAbs®, TandAbs®, Fab derivatives), cyclophilin inhibitors, stimulators of retinoic acid-inducible gene 1, PD-1 inhibitors, PD-L1 inhibitors, Arginase-1 inhibitors, PI3K inhibitors and stimulators of NOD2, and one or two additional therapeutic agents selected from the group consisting of: HBV viral entry inhibitors, NTCP inhibitors, HBx inhibitors, cccDNA inhibitors, HBV antibodies targeting the surface antigens of the hepatitis B virus, short interfering RNAs (siRNA), miRNA gene therapy agents, short synthetic hairpin RNAs (sshRNAs), and nucleoprotein inhibitors (HBV core or capsid protein inhibitors). In certain embodiments one or two additional therapeutic agents is further selected from hepatitis B surface antigen (HBsAg) secretion or assembly inhibitors, TCR-like antibodies, and IDO inhibitors.

In a particular embodiment, a compound of the present disclosure, or a pharmaceutically acceptable salt thereof, is combined with one, two, three, four or more additional therapeutic agents selected from adefovir (Hepsera®), tenofovir disoproxil funarate+emtricitabine (TRUVADA®), tenofovir disoproxil fumarate (Viread®), entecavir (Baraclude®), lamivudine (Epivir-HBV®), tenofovir alafenamide, tenofovir, tenofovir disoproxil, tenofovir alafenamide fumarate, tenofovir alafenamide hemifumarate, telbivudine (Tyzeka®), Clevudine®, emtricitabine (Emtriva®), peginterferon alfa-2b (PEG-Intron®), Multiferon®, interferon alpha 1b (Hapgen®), interferon alpha-2b (Intron A®), pegylated interferon alpha-2a (Pegasys®), interferon alfa-n1 (Humoferon®), ribavirin, interferon beta-1a (Avonex®), Bioferon, Ingaron, Inmutag (Inferon), Algeron, Roferon-A, Oligotide, Zutectra, Shaferon, interferon alfa-2b (AXXO), Alfaferone, interferon alfa-2b (BioGeneric Pharma), Feron, interferon-alpha 2 (CJ), BEVAC, Laferonum, VIPEG, BLAUFERON-B, BLAUFERON-A, Intermax Alpha, Realdiron, Lanstion, Pegaferon, PDferon-B, interferon alfa-2b (IFN, Laboratorios Bioprofarma), alfainterferona 2b, Kalferon, Pegnano, Feronsure, PegiHep, interferon alfa 2b (Zydus-Cadila), Optipeg A, Realfa 2B, Reliferon, interferon alfa-2b (Amega), interferon alfa-2b (Virchow), peginterferon alfa-2b (Amega), Reaferon-EC, Proquiferon, Uniferon, Urifron, interferon alfa-2b (Changchun Institute of Biological Products), Anterferon, Shanferon, MOR-22, interleukin-2 (IL-2, Immunex), recombinant human interleukin-2 (Shenzhen Neptunus), Layfferon, Ka Shu Ning, Shang Sheng Lei Tai, INTEFEN, SINOGEN, Fukangtai, Alloferon and celmoleukin

In a particular embodiment, a compound of the present disclosure, or a pharmaceutically acceptable salt thereof, is combined with entecavir (Baraclude®), adefovir (Hepsera®), tenofovir disoproxil fumarate (Viread®), tenofovir alafenamide, tenofovir, tenofovir disoproxil, tenofovir alafenamide fumarate, tenofovir alafenamide hemifumarate, telbivudine (Tyzeka®) or lamivudine (Epivir-HBV®)

In a particular embodiment, a compound of the present disclosure, or a pharmaceutically acceptable salt thereof, is combined with entecavir (Baraclude®), adefovir (Hepsera®), tenofovir disoproxil fumarate (Viread®), tenofovir alafenamide hemifumarate, telbivudine (Tyzeka®) or lamivudine (Epivir-HBV®).

In a particular embodiment, a compound of the present disclosure, or a pharmaceutically acceptable salt thereof is combined with a PD-1 inhibitor. In a particular embodiment, a compound of the present disclosure, or a pharmaceutically acceptable salt thereof is combined with a PD-L1 inhibitor. In a particular embodiment, a compound of the present disclosure, or a pharmaceutically acceptable salt thereof is combined with an IDO inhibitor. In a particular embodiment, a compound of the present disclosure, or a pharmaceutically acceptable salt thereof is combined with an IDO inhibitor and a PD-1 inhibitor. In a particular embodiment, a compound of the present disclosure, or a pharmaceutically acceptable salt thereof, is combined with an IDO inhibitor and a PD-L1 inhibitor. In a particular embodiment, a compound of the present disclosure, or a pharmaceutically acceptable salt thereof, is combined with a TLR7 modulator, such as GS-9620.

In a particular embodiment, a compound of the present disclosure, or a pharmaceutically acceptable salt thereof, is combined with a TLR7 modulator and an IDO inhibitor. In a particular embodiment, a compound of the present disclosure, or a pharmaceutically acceptable salt thereof, is combined with a TLR7 modulator such as GS-9620 and an IDO inhibitor such as epacadostat.

In a particular embodiment, a compound of the present disclosure, or a pharmaceutically acceptable salt thereof, is combined with (4-amino-2-butoxy-8-({3-[(pyrrolidin-1-yl)methyl]phenyl}methyl)-7,8-dihydropteridin-6(5H)-one) or a pharmaceutically acceptable salt thereof.

As used herein, GS-9620 (4-amino-2-butoxy-8-({3-[(pyrrolidin-1-yl)methyl]phenyl}methyl)-7,8-dihydropteridin-6(5H)-one), includes pharmaceutically acceptable salts thereof. J. Med. Chem., 2013, 56 (18), pp 7324-7333.

In a particular embodiment, a compound of the present disclosure, or a pharmaceutically acceptable salt thereof, is combined with a first additional therapeutic agent selected from the group consisting of: entecavir (Baraclude®), adefovir (Hepsera®), tenofovir disoproxil fumarate (Viread®), tenofovir alafenamide, tenofovir, tenofovir disoproxil, tenofovir alafenamide fumarate, tenofovir alafenamide hemifumarate, telbivudine (Tyzeka®) or lamivudine (Epivir-HBV®) and at least one additional therapeutic agent selected from the group consisting of immunomodulators, toll-like receptor modulators (modulators of TLR-1, TLR-2, TLR-3, TLR-4, TLR-5, TLR-6, TLR-7, TLR-8, TLR-9, TLR-10, TLR-11, TLR-12 and TLR-13), interferon alpha receptor ligands, hyaluronidase inhibitors, recombinant IL-7, HBsAg inhibitors, compounds targeting HbcAg, cyclophilin inhibitors, HBV Therapeutic vaccines, HBV prophylactic vaccines, HBV viral entry inhibitors, NTCP inhibitors, antisense oligonucleotide targeting viral mRNA, short interfering RNAs (siRNA), miRNA gene therapy agents, endonuclease modulators, inhibitors of ribonucleotide reductase, Hepatitis B virus E antigen inhibitors, recombinant scavenger receptor A (SRA) proteins, src kinase inhibitors, HBx inhibitors, cccDNA inhibitors, short synthetic hairpin RNAs (sshRNAs), HBV antibodies including HBV antibodies targeting the surface antigens of the hepatitis B virus and bispecific antibodies and “antibody-like” therapeutic proteins (such as DARTs®, Duobodies®, Bites®, XmAbs®, TandAbs®, Fab derivatives), CCR2 chemokine antagonists, thymosin agonists, cytokines, nucleoprotein inhibitors (HBV core or capsid protein inhibitors), stimulators of retinoic acid-inducible gene 1, stimulators of NOD2, stimulators of NOD1, recombinant thymosin alpha-1, Arginase-1 inhibitors, STING agonists, PI3K inhibitors, lymphotoxin beta receptor activators, Natural Killer Cell Receptor 2B4 inhibitors, Lymphocyte-activation gene 3 inhibitors, CD160 inhibitors, cytotoxic T-lymphocyte-associated protein 4 inhibitors, CD137 inhibitors, Killer cell lectin-like receptor subfamily G member 1 inhibitors, TIM-3 inhibitors, B- and T-lymphocyte attenuator inhibitors, CD305 inhibitors, PD-1 inhibitors, PD-L1 inhibitors, PEG-Interferon Lambd, BTK inhibitors, modulators of TIGIT, modulators of CD47, modulators of SIRPalpha, modulators of ICOS, modulators of CD27, modulators of CD70, modulators of OX40, modulators of NKG2D, modulators of Tim-4, modulators of B7-H4, modulators of B7-H3, modulators of NKG2A, modulators of GITR, modulators of CD160, modulators of HEVEM, modulators of CD161, modulators of Axl, modulators of Mer, modulators of Tyro, gene modifiers or editors such as CRISPR (including CRISPR Cas9), zinc finger nucleases or synthetic nucleases (TALENs), a and Hepatitis B virus replication inhibitors. In certain embodiments, the at least one additional therapeutic agent is further selected from hepatitis B surface antigen (HBsAg) secretion or assembly inhibitors, TCR-like antibodies, IDO inhibitors, cccDNA epigenetic modifiers, IAPs inhibitors, and SMAC mimetics.

In a particular embodiment, a compound of the present disclosure, or a pharmaceutically acceptable salt thereof, is combined with a first additional therapeutic agent selected from the group consisting of: entecavir (Baraclude®), adefovir (Hepsera®), tenofovir disoproxil fumarate (Viread®), tenofovir alafenamide, tenofovir, tenofovir disoproxil, tenofovir alafenamide fumarate, tenofovir alafenamide hemifumarate, telbivudine (Tyzeka®) or lamivudine (Epivir-HBV®) and at least a one additional therapeutic agent selected from the group consisting of peginterferon alfa-2b (PEG-Intron®), Multiferon®, interferon alpha 1b (Hapgen®), interferon alpha-2b (Intron A®), pegylated interferon alpha-2a (Pegasys®), interferon alfa-n1 (Humoferon®), ribavirin, interferon beta-1a (Avonex®), Bioferon, Ingaron, Inmutag (Inferon), Algeron, Roferon-A, Oligotide, Zutectra, Shaferon, interferon alfa-2b (AXXO), Alfaferone, interferon alfa-2b (BioGeneric Pharma), Feron, interferon-alpha 2 (CJ), BEVAC, Laferonum, VIPEG, BLAUFERON-B, BLAUFERON-A, Intermax Alpha, Realdiron, Lanstion, Pegaferon, PDferon-B, interferon alfa-2b (IFN, Laboratorios Bioprofarma), alfainterferona 2b, Kalferon, Pegnano, Feronsure, PegiHep, interferon alfa 2b (Zydus-Cadila), Optipeg A, Realfa 2B, Reliferon, interferon alfa-2b (Amega), interferon alfa-2b (Virchow), peginterferon alfa-2b (Amega), Reaferon-EC, Proquiferon, Uniferon, Urifron, interferon alfa-2b (Changchun Institute of Biological Products), Anterferon, Shanferon, MOR-22, interleukin-2 (IL-2, Immunex), recombinant human interleukin-2 (Shenzhen Neptunus), Layfferon, Ka Shu Ning, Shang Sheng Lei Tai, INTEFEN, SINOGEN, Fukangtai, Alloferon and celmoleukin.

In a particular embodiment, a compound of the present disclosure, or a pharmaceutically acceptable salt thereof, is combined with a first additional therapeutic agent selected from the group consisting of: entecavir (Baraclude®), adefovir (Hepsera®), tenofovir disoproxil fumarate (Viread®), tenofovir alafenamide, tenofovir, tenofovir disoproxil, tenofovir alafenamide fumarate, tenofovir alafenamide hemifumarate, telbivudine (Tyzeka®) or lamivudine (Epivir-HBV®) and at least one additional therapeutic agent selected from the group consisting of HBV viral entry inhibitors, NTCP inhibitors, HBx inhibitors, cccDNA inhibitors, HBV antibodies targeting the surface antigens of the hepatitis B virus, short interfering RNAs (siRNA), miRNA gene therapy agents, short synthetic hairpin RNAs (sshRNAs), and nucleoprotein inhibitors (HBV core or capsid protein inhibitors).

In a particular embodiment, a compound of the present disclosure, or a pharmaceutically acceptable salt thereof, is combined with a first additional therapeutic agent selected from the group consisting of: entecavir (Baraclude®), adefovir (Hepsera®), tenofovir disoproxil fumarate (Viread®), tenofovir alafenamide, tenofovir, tenofovir disoproxil, tenofovir alafenamide fumarate, tenofovir alafenamide hemifumarate, telbivudine (Tyzeka®) or lamivudine (Epivir-HBV®), one or two additional therapeutic agents selected from the group consisting of: immunomodulators, toll-like receptor modulators (modulators of TLR-1, TLR-2, TLR-3, TLR-4, TLR-5, TLR-6, TLR-7, TLR-8, TLR-9, TLR-10, TLR-11, TLR-12 and TLR-13), HBsAg inhibitors, HBV therapeutic vaccines, HBV antibodies including HBV antibodies targeting the surface antigens of the hepatitis B virus and bispecific antibodies and “antibody-like” therapeutic proteins (such as DARTs®, Duobodies®, Bites®, XmAbs®, TandAbs®, Fab derivatives), cyclophilin inhibitors, stimulators of retinoic acid-inducible gene 1, PD-1 inhibitors, PD-L1 inhibitors, Arginase-1 inhibitors, PI3K inhibitors and stimulators of NOD2, and one or two additional therapeutic agents selected from the group consisting of: HBV viral entry inhibitors, NTCP inhibitors, HBx inhibitors, cccDNA inhibitors, HBV antibodies targeting the surface antigens of the hepatitis B virus, short interfering RNAs (siRNA), miRNA gene therapy agents, short synthetic hairpin RNAs (sshRNAs), and nucleoprotein inhibitors (HBV core or capsid protein inhibitors). In certain embodiments, the one or two additional therapeutic agents is further selected from hepatitis B surface antigen (HBsAg) secretion or assembly inhibitors, TCR-like antibodies, and IDO inhibitors.

In certain embodiments, a compound of the present disclosure, or a pharmaceutically acceptable salt thereof, is combined with 5-30 mg tenofovir alafenamide fumarate, tenofovir alafenamide hemifumarate, or tenofovir alafenamide. In certain embodiments, a compound of the present disclosure, or a pharmaceutically acceptable salt thereof, is combined with 5-10; 5-15; 5-20; 5-25; 25-30; 20-30; 15-30; or 10-30 mg tenofovir alafenamide fumarate, tenofovir alafenamide hemifumarate, or tenofovir alafenamide. In certain embodiments, a compound of the present disclosure, or a pharmaceutically acceptable salt thereof, is combined with 10 mg tenofovir alafenamide fumarate, tenofovir alafenamide hemifumarate, or tenofovir alafenamide. In certain embodiments, a compound of the present disclosure, or a pharmaceutically acceptable salt thereof, is combined with 25 mg tenofovir alafenamide fumarate, tenofovir alafenamide hemifumarate, or tenofovir alafenamide. A compound of the present disclosure (e.g., a compound of Formula (I)) may be combined with the agents provided herein in any dosage amount of the compound (e.g., from 50 mg to 500 mg of compound) the same as if each combination of dosages were specifically and individually listed. A compound of the present disclosure (e.g., a compound of Formula (I)) may be combined with the agents provided herein in any dosage amount of the compound (e.g. from about 1 mg to about 150 mg of compound) the same as if each combination of dosages were specifically and individually listed.

In certain embodiments, a compound of the present disclosure, or a pharmaceutically acceptable salt thereof, is combined with 100-400 mg tenofovir disoproxil fumarate, tenofovir disoproxil hemifumarate, or tenofovir disoproxil. In certain embodiments, a compound of the present disclosure, or a pharmaceutically acceptable salt thereof, is combined with 100-150; 100-200, 100-250; 100-300; 100-350; 150-200; 150-250; 150-300; 150-350; 150-400; 200-250; 200-300; 200-350; 200-400; 250-350; 250-400; 350-400 or 300-400 mg tenofovir disoproxil fumarate, tenofovir disoproxil hemifumarate, or tenofovir disoproxil. In certain embodiments, a compound of the present disclosure, or a pharmaceutically acceptable salt thereof, is combined with 300 mg tenofovir disoproxil fumarate, tenofovir disoproxil hemifumarate, or tenofovir disoproxil. In certain embodiments, a compound of the present disclosure, or a pharmaceutically acceptable salt thereof, is combined with 250 mg tenofovir disoproxil fumarate, tenofovir disoproxil hemifumarate, or tenofovir disoproxil. In certain embodiments, a compound of the present disclosure, or a pharmaceutically acceptable salt thereof, is combined with 150 mg tenofovir disoproxil fumarate, tenofovir disoproxil hemifumarate, or tenofovir disoproxil. A compound of the present disclosure (e.g., a compound of Formula (I)) may be combined with the agents provided herein in any dosage amount of the compound (e.g., from 50 mg to 500 mg of compound) the same as if each combination of dosages were specifically and individually listed. A compound of the present disclosure (e.g., a compound of Formula (I)) may be combined with the agents provided herein in any dosage amount of the compound (e.g., from about 1 mg to about 150 mg of compound) the same as if each combination of dosages were specifically and individually listed.

Also provided herein is a compound of the present disclosure (e.g., a compound of Formula (I)), or a pharmaceutically acceptable salt thereof, and one or more additional active ingredients for treating HBV, for use in a method of treating or preventing HBV.

Also provided herein is a compound of the present disclosure (e.g., a compound of Formula (I)), or a pharmaceutically acceptable salt thereof, for use in a method of treating or preventing HBV, wherein the compound, or a pharmaceutically acceptable salt thereof is administered simultaneously, separately or sequentially with one or more additional therapeutic agents fort for treating HBV.

### VIII. Combination Therapy for HCV

In certain embodiments, a method for treating or preventing an HCV infection in a human having or at risk of having the infection is provided, comprising administering to the human a therapeutically effective amount of a compound of the present disclosure, or a pharmaceutically acceptable salt thereof, in combination with a therapeutically effective amount of one or more (e.g., one, two, three, one or two, or one to three) additional therapeutic agents. In one embodiment, a method for treating an HCV infection in a human having or at risk of having the infection is provided, comprising administering to the human a therapeutically effective amount of a compound of the present disclosure, or a pharmaceutically acceptable salt thereof, in combination with a therapeutically effective amount of one or more (e.g., one, two, three, one or two, or one to three) additional therapeutic agents.

In certain embodiments, the present disclosure provides a method for treating an HCV infection, comprising administering to a patient in need thereof a therapeutically effective amount of a compound of the present disclosure, or a pharmaceutically acceptable salt thereof, in combination with a therapeutically effective amount of one or more additional therapeutic agents which are suitable for treating an HCV infection.

In the above embodiments, the additional therapeutic agent may be an anti-HCV agent. For example, in some embodiments, the additional therapeutic agent is selected from the group consisting of interferons, ribavirin or its analogs, HCV NS3 protease inhibitors, HCV NS4 protease inhibitors, HCV NS3/NS4 protease inhibitors, alpha-glucosidase 1 inhibitors, hepatoprotectants, nucleoside or nucleotide inhibitors of HCV NS5B polymerase, non-nucleoside inhibitors of HCV NS5B polymerase, HCV NS5A inhibitors, TLR-7 agonists, cyclophilin inhibitors, HCV IRES inhibitors, and pharmacokinetic enhancers, compounds such as those disclosed in US2010/0310512, US2013/0102525, and WO2013/185093, or combinations thereof.

In certain embodiments a compound of the present disclosure (e.g., a compound of Formula (I)) is formulated as a tablet, which may optionally contain one or more other compounds useful for treating HCV. In certain embodiments, the tablet can contain another active ingredient for treating HCV, such as interferons, ribavirin or its analogs, HCV NS3 protease inhibitors, HCV NS4 protease inhibitors, HCV NS3/NS4 protease inhibitors, alpha-glucosidase 1 inhibitors, hepatoprotectants, nucleoside or nucleotide inhibitors of HCV NS5B polymerase, non-nucleoside inhibitors of HCV NS5B polymerase, HCV NS5A inhibitors, TLR-7 agonists, cyclophilin inhibitors, HCV IRES inhibitors, and pharmacokinetic enhancers, or combinations thereof.

In certain embodiments, such tablets are suitable for once daily dosing.

In certain embodiments, the additional therapeutic agent is selected from one or more of:

(1) Interferons selected from the group consisting of pegylated rIFN-alpha 2b (PEG-Intron), pegylated rIFN-alpha 2a (Pegasys), rIFN-alpha 2b (Intron A), rIFN-alpha 2a (Roferon-A), interferon alpha (MOR-22, OPC-18, Alfaferone, Alfanative, Multiferon, subalin), interferon alfacon-1 (Infergen), interferon alpha-n1 (Wellferon), interferon alpha-n3 (Alferon), interferon-beta (Avonex, DL-8234), interferon-omega (omega DUROS, Biomed 510), albinterferon alpha-2b (Albuferon), IFN alpha XL, BLX-883 (Locteron), DA-3021, glycosylated interferon alpha-2b (AVI-005), PEG-Infergen, PEGylated interferon lambda (PEGylated IL-29), or belerofon, IFN alpha-2b XL, rIFN-alpha 2a, consensus IFN alpha, infergen, rebif, pegylated IFN-beta, oral interferon alpha, feron, reaferon, intermax alpha, r-IFN-beta, and infergen+actimmuneribavirin and ribavirin analogs, e.g., rebetol, copegus, VX-497, and viramidine (taribavirin);
(2) Ribavirin and its analogs selected from the group consisting of ribavirin (Rebetol, Copegus), and taribavirin (Viramidine);
(3) NS5A inhibitors selected from the group consisting of Compound A.1 (described below), Compound A.2 (described below), Compound A.3 (described below), ABT-267, Compound A.4 (described below), JNJ-47910382, daclatasvir (BMS-790052), ABT-267, Samatasvir, MK-8742, MK-8404, EDP-239, IDX-719, PPI-668, GSK-2336805, ACH-3102, A-831, A-689, AZD-2836 (A-831), AZD-7295 (A-689), and BMS-790052;
(4) NS5B polymerase inhibitors selected from the group consisting of sofosbuvir (GS-7977), Compound A.5 (described below), Compound A.6 (described below), ABT-333, Compound A.7 (described below), ABT-072, Compound A.8 (described below), tegobuvir (GS-9190), GS-9669, TMC647055, ABT-333, ABT-072, setrobuvir (ANA-598), IDX-21437, filibuvir (PF-868554), VX-222, IDX-375, IDX-184, IDX-102, BI-207127, valopicitabine (NM-283), PSI-6130 (R1656), PSI-7851, BCX-4678, nesbuvir (HCV-796), BILB 1941, MK-0608, NM-107, R7128, VCH-759, GSK625433, XTL-2125, VCH-916, JTK-652, MK-3281, VBY-708, A848837, GL59728, A-63890, A-48773, A-48547, BC-2329, BMS-791325, BILB-1941, AL-335, AL-516 and ACH-3422;
(5) Protease (NS3, NS3-NS4) inhibitors selected from the group consisting of Compound A.9, Compound A.10, Compound A.11, ABT-450, Compound A.12 (described below), simeprevir (TMC-435), boceprevir (SCH-503034), narlaprevir (SCH-900518), vaniprevir (MK-7009), MK-5172, danoprevir (ITMN-191), sovaprevir (ACH-1625), neceprevir (ACH-2684), Telaprevir (VX-950), VX-813, VX-500, faldaprevir (BI-201335), asunaprevir (BMS-650032), BMS-605339, VBY-376, PHX-1766, YH5531, BILN-2065, and BILN-2061;
(6) Alpha-glucosidase 1 inhibitors selected from the group consisting of celgosivir (MX-3253), Miglitol, and UT-231B;
(7) Hepatoprotectants selected from the group consisting of emericasan (IDN-6556), ME-3738, GS-9450 (LB-84451), silibilin, and MitoQ;
(8) TLR-7 agonists selected from the group consisting of imiquimod, 852A, GS-9524, ANA-773, ANA-975, AZD-8848 (DSP-3025), and SM-360320;
(9) Cyclophilin inhibitors selected from the group consisting of DEBIO-025, SCY-635, and NIM811;
(10) HCV IRES inhibitors selected from the group consisting of MCI-067;
(11) Pharmacokinetic enhancers selected from the group consisting of BAS-100, SPI-452, PF-4194477, TMC-41629, GS-9350, GS-9585, and roxythromycin; and (12) Other anti-HCV agents selected from the group consisting of thymosin alpha 1 (Zadaxin), nitazoxanide (Alinea, NTZ), BIVN-401 (virostat), PYN-17 (altirex), KPE02003002, actilon (CPG-10101), GS-9525, KRN-7000, civacir, GI-5005, XTL-6865, BIT225, PTX-111, ITX2865, TT-033i, ANA 971, NOV-205, tarvacin, EHC-18, VGX-410C, EMZ-702, AVI 4065, BMS-650032, BMS-791325, Bavituximab, MDX-1106 (ONO-4538), Oglufanide, VX-497 (merimepodib) NIM811, benzimidazole derivatives, benzo-1,2,4-thiadiazine derivatives, and phenylalanine derivatives;

Compound A.1 is an inhibitor of the HCV NS5A protein and is represented by the following chemical structure:

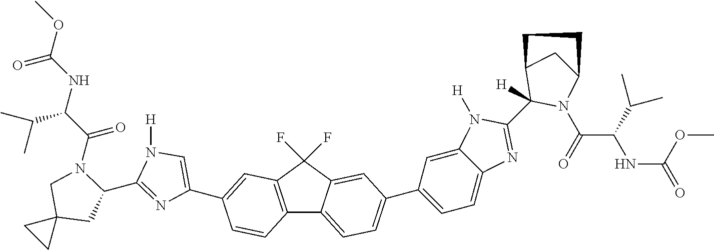

(see, e.g., U.S. Application Publication No. 20100310512 A1).

Compound A.2 is an NS5A inhibitor and is represented by the following chemical structure:

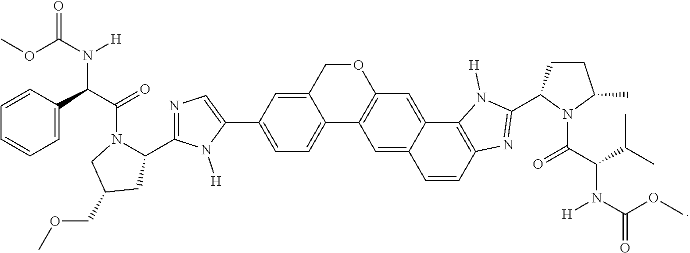 [SMILES: COC[C@H]1C[C@@H](c2ncc(-c3ccc4c(c3)COc3cc5c(ccc6nc([C@@H]7CC[C@H](C)N7C(=O)[C@@H](NC(=O)OC)C(C)C)[nH]c65)cc3-4)[nH]2)N(C(=O)[C@H](NC(=O)OC)c2ccccc2)C1]

Compound A.3 is an NS5A inhibitor and is represented by the following chemical structure:

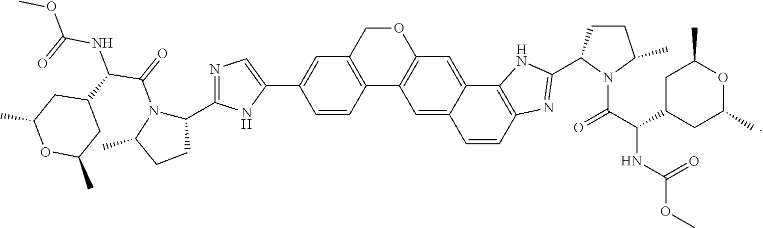

Compound A.4 is an NS5A inhibitor and is represented by the following chemical structure:

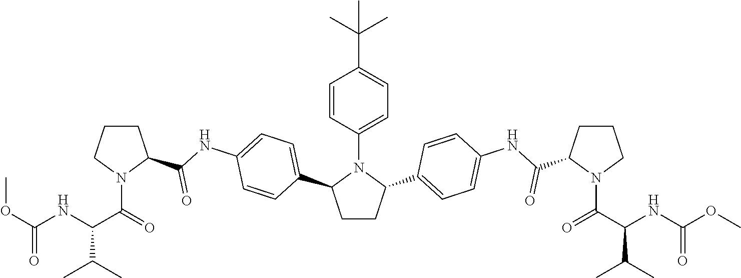 [SMILES: COC(=O)N[C@H](C(=O)N1CCC[C@H]1C(=O)Nc1ccc([C@@H]2CC[C@@H](c3ccc(NC(=O)[C@@H]4CCCN4C(=O)[C@@H](NC(=O)OC)C(C)C)cc3)N2c2ccc(C(C)(C)C)cc2)cc1)C(C)C]

(see U.S. Application Publication No. 2013/0102525 and references therein.)

Compound A.5 is a NS5B Thumb II polymerase inhibitor and is represented by the following chemical structure:

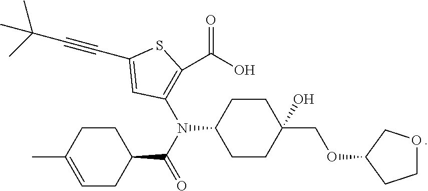 [SMILES: CC1=CC[C@H](C(=O)N(c2cc(C#CC(C)(C)C)sc2C(=O)O)[C@H]2CC[C@](O)(CO[C@H]3CCOC3)CC2)CC1]

Compound A.6 is a nucleotide inhibitor prodrug designed to inhibit replication of viral RNA by the HCV NS5B polymerase, and is represented by the following chemical structure:

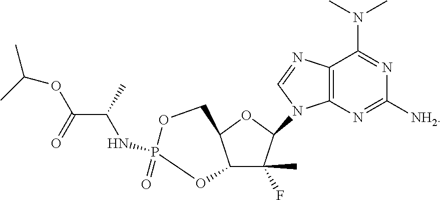 [SMILES: CC(C)OC(=O)[C@H](C)N[P@]1(=O)OC[C@H]2O[C@@H](n3cnc4c(N(C)C)nc(N)nc43)[C@](C)(F)[C@@H]2O1]

Compound A.7 is an HCV polymerase inhibitor and is represented by the following structure:

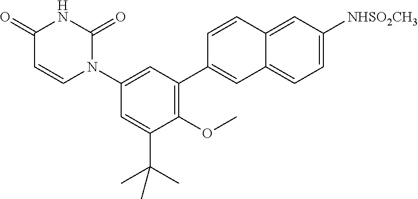

(see U.S. Application Publication No. 2013/0102525 and references therein).

Compound A.8 is an HCV polymerase inhibitor and is represented by the following structure:

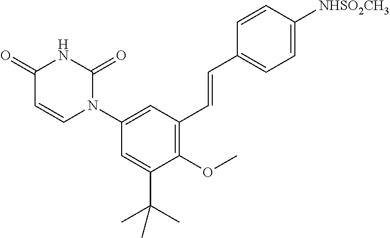

(see U.S. Application Publication No. 2013/0102525 and references therein).

Compound A.9 is an HCV protease inhibitor and is represented by the following chemical structure:

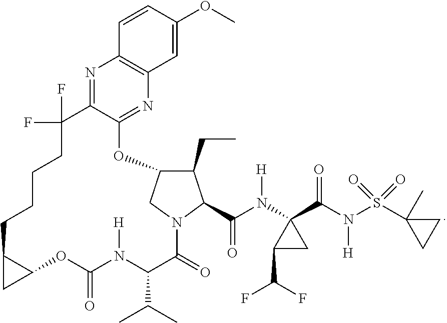 [SMILES: CC[C@@H]1[C@@H]2CN(C(=O)[C@H](C(C)C)NC(=O)O[C@@H]3C[C@H]3CCCCC(F)(F)c3nc4ccc(OC)cc4nc3O2)[C@@H]1C(=O)N[C@]1(C(=O)NS(=O)(=O)C2(C)CC2)C[C@H]1C(F)F]

Compound A.10 is an HCV protease inhibitor and is represented by the following chemical structure:

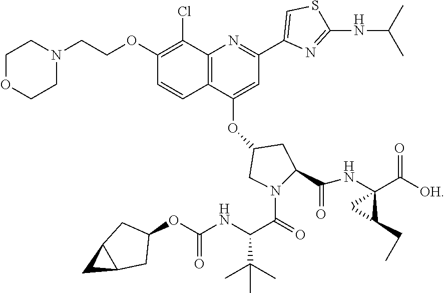

Compound A.11 is an HCV protease inhibitor and is represented by the following chemical structure:

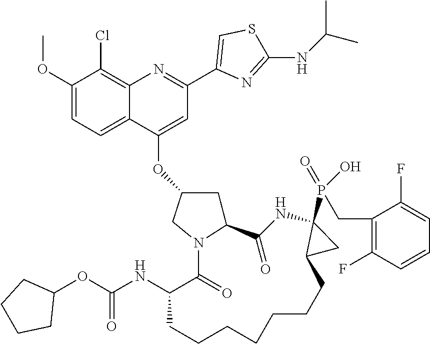 [SMILES: COc1ccc2c(O[C@@H]3C[C@H]4C(=O)N[C@]5(P(=O)(O)Cc6c(F)cccc6F)C[C@H]5CCCCCCC[C@H](NC(=O)OC5CCCC5)C(=O)N4C3)cc(-c3csc(NC(C)C)n3)nc2c1Cl]

Compound A.12 is an HCV protease inhibitor and is represented by the following chemical structure:

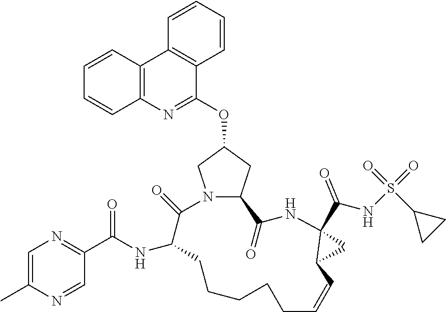 [SMILES: Cc1cnc(C(=O)N[C@H]2CCCCC/C=C\[C@@H]3C[C@@]3(C(=O)NS(=O)(=O)C3CC3)NC(=O)[C@@H]3C[C@@H](Oc4nc5ccccc5c5ccccc45)CN3C2=O)cn1]

(see U.S. Application Publication No. 2013/0102525 and references therein).

In one embodiment, the additional therapeutic agent used in combination with the pharmaceutical compositions as described herein is a HCV NS3 protease inhibitor. Non-limiting examples include the following:

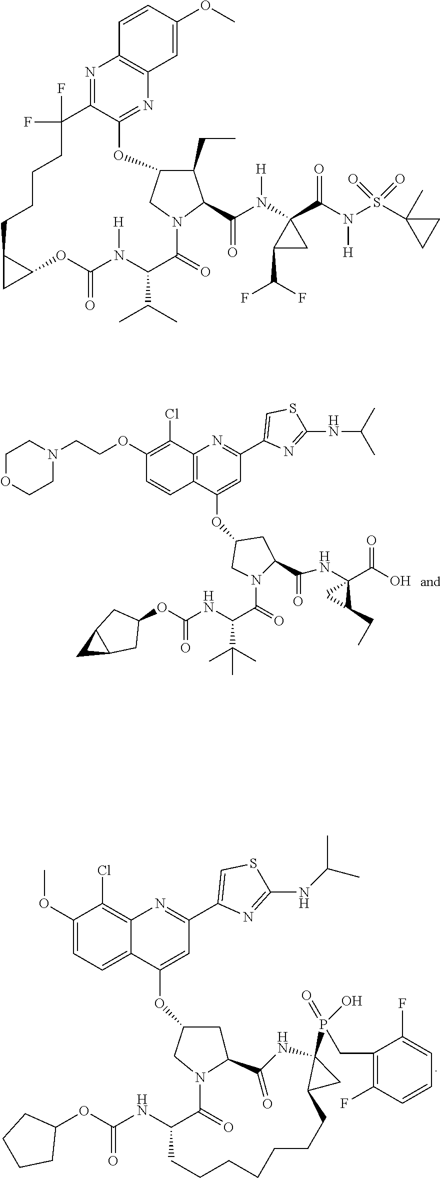

In another embodiment, the additional therapeutic agent used in combination with the pharmaceutical compositions as described herein is a cyclophilin inhibitor, including for example, a cyclophilin inhibitor disclosed in WO2013/185093. Non-limiting examples in addition to those listed above include the following:

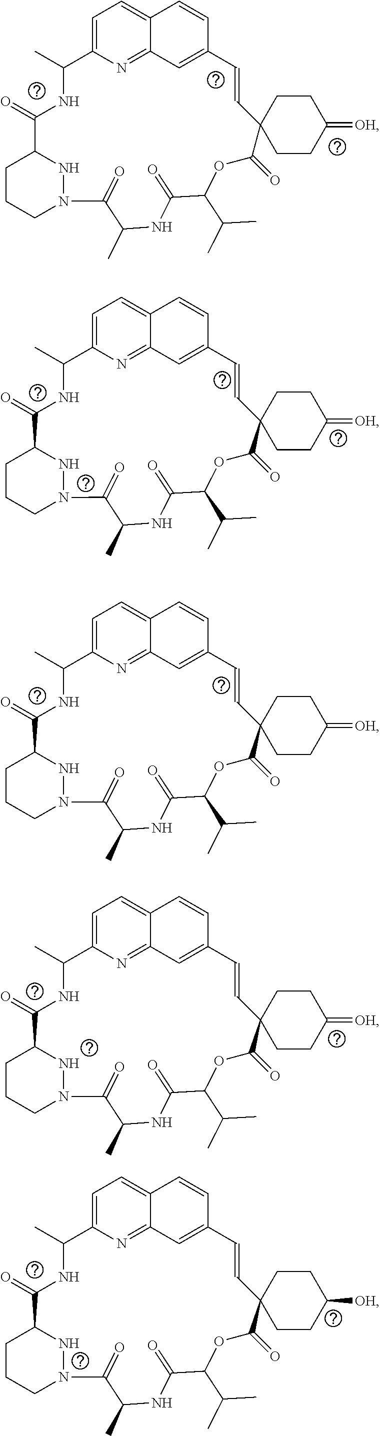

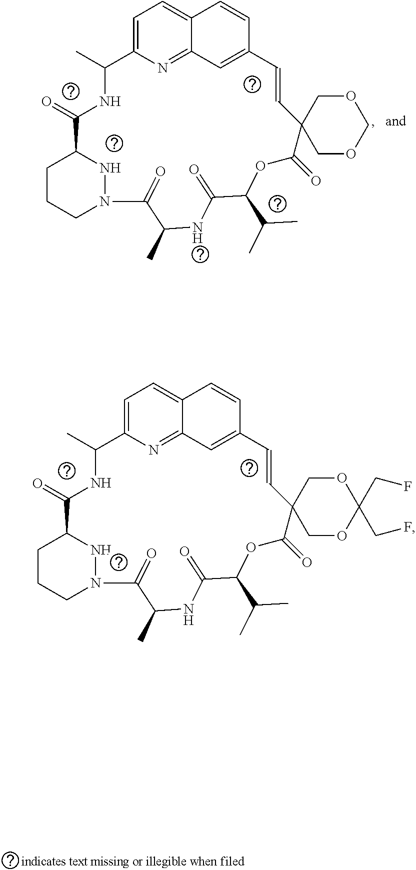 [SMILES: CC1NC(=O)[C@@H]2CCCN(N2)C(=O)[C@H](C)NC(=O)[C@H](C(C)C)OC(=O)C2(/C=C/c3ccc4ccc1nc4c3)COCOC2 ; CC1NC(=O)[C@@H]2CCCN(N2)C(=O)[C@H](C)NC(=O)[C@H](C(C)C)OC(=O)C2(/C=C/c3ccc4ccc1nc4c3)COC(CF)(CF)OC2]

and stereoisomers and mixtures of stereoisomers thereof.

In a specific embodiment, a compound of the present disclosure, or a pharmaceutically acceptable salt thereof, is combined with a HCV NS5B polymerase inhibitor. In a specific embodiment, a compound of the present disclosure, or a pharmaceutically acceptable salt thereof, is combined with a HCV NS5B polymerase inhibitor and a HCV NS5A inhibitor. In another specific embodiment, a compound of the present disclosure, or a pharmaceutically acceptable salt thereof, is combined with a HCV NS5B polymerase inhibitor, a HCV NS3 protease inhibitor and a HCV NS5A inhibitor. In another specific embodiment, a compound of the present disclosure, or a pharmaceutically acceptable salt thereof, is combined with a HCV NS5B polymerase inhibitor, a HCV NS4 protease inhibitor and a HCV NS5A inhibitor. In another specific embodiment, a compound of the present disclosure, or a pharmaceutically acceptable salt thereof, is combined with a HCV NS5B polymerase inhibitor, a HCV NS3/NS4 protease inhibitor and a HCV NS5A inhibitor. In another specific embodiment, a compound of the present disclosure, or a pharmaceutically acceptable salt thereof, is combined with a HCV NS3 protease inhibitor and a HCV NS5A inhibitor. In another specific embodiment, a compound of the present disclosure, or a pharmaceutically acceptable salt thereof, is combined with a HCV NS4 protease inhibitor and a HCV NS5A inhibitor. In another specific embodiment, a compound of the present disclosure, or a pharmaceutically acceptable salt thereof, is combined with a HCV NS3/NS4 protease inhibitor and a HCV NS5A inhibitor. In another specific embodiment, a compound of the present disclosure, or a pharmaceutically acceptable salt thereof, is combined with a HCV NS3 protease inhibitor, a pharmacokinetic enhancer and a HCV NS5A inhibitor. In another specific embodiment, a compound of the present disclosure, or a pharmaceutically acceptable salt thereof, is combined with a HCV NS4 protease inhibitor, a pharmacokinetic enhancer and a HCV NS5A inhibitor. In another specific embodiment, a compound of the present disclosure, or a pharmaceutically acceptable salt thereof, is combined with a HCV NS3/NS4 protease inhibitor, a pharmacokinetic enhancer and a HCV NS5A inhibitor.

In a particular embodiment, a compound of the present disclosure, or a pharmaceutically acceptable salt thereof, is combined with one, two, three, four or more additional therapeutic agents selected from simeprevir, MK-8742, MK-8408, MK-5172, ABT-450, ABT-267, ABT-333, sofosbuvir, sofosbuvir+ledipasvir, sofosbuvir+GS-5816, sofosbuvir+GS-9857+ledipasvir, ABT-450+ABT-267+ritonavir, ABT-450+ABT-267+ribavirin+ritonavir, ABT-450+ABT-267+ribavirin+ABT-333+ritonavir, ABT-530+ABT-493, MK-8742+MK-5172, MK-8408+MK-3682+MK-5172, MK-8742+MK-3682+MK-5172, daclatasvir, interferon, pegylated interferon, ribavirin, samatasvir, MK-3682, ACH-3422, AL-335, IDX-21437, IDX-21459, tegobuvir, setrobuvir, valopicitabine, boceprevir, narlaprevir, vaniprevir, danoprevir, sovaprevir, neceprevir, telaprevir, faldaprevir, asunaprevir, ledipasvir, GS-5816, GS-9857, ACH-3102, ACH-3422+ACH-3102, ACH-3422+sovaprevir+ACH-3102, asunaprevir, asunaprevir+daclatasvir, AL-516, and vedroprevir.

In certain embodiments, a compound of the present disclosure, or a pharmaceutically acceptable salt thereof, is co-administered with simeprevir. In certain embodiments, a compound of the present disclosure, or a pharmaceutically acceptable salt thereof, is co-administered with MK-8742 or MK-8408. In certain embodiments, a compound of the present disclosure, or a pharmaceutically acceptable salt thereof, is co-administered with MK-5172. In certain embodiments, a compound of the present disclosure, or a pharmaceutically acceptable salt thereof, is co-administered with ABT-450, ABT-267, or ABT-333. In certain embodiments, a compound of the present disclosure, or a pharmaceutically acceptable salt thereof, is co-administered with Viekirat (a combination of ABT-450, ABT-267, and ritonavir). In certain embodiments, a compound of the present disclosure, or a pharmaceutically acceptable salt thereof, is co-administered with daclatasvir. In certain embodiments, a compound of the present disclosure, or a pharmaceutically acceptable salt thereof, is co-administered with sofosbuvir. In certain embodiments, a compound of the present disclosure, or a pharmaceutically acceptable salt thereof, is co-administered with Harvoni (sofosbuvir+ledipasvir). In certain embodiments, a compound of the present disclosure, or a pharmaceutically acceptable salt thereof, is co-administered with sofosbuvir and GS-5816. In certain embodiments, a compound of the present disclosure, or a pharmaceutically acceptable salt thereof, is co-administered with sofosbuvir+GS-9857+ledipasvir. In certain embodiments, a compound of the present disclosure, or a pharmaceutically acceptable salt thereof, is co-administered with ABT-450+ABT-267+ribavirin+ritonavir. In certain embodiments, a compound of the present disclosure, or a pharmaceutically acceptable salt thereof, is co-administered with ABT-450+ABT-267+ribavirin+ABT-333+ritonavir. In certain embodiments, a compound of the present disclosure, or a pharmaceutically acceptable salt thereof, is co-administered with ABT-530+ABT-493. In certain embodiments, a compound of the present disclosure, or a pharmaceutically acceptable salt thereof, is co-administered with MK-8408+MK-3682+MK-5172. In certain embodiments, a compound of the present disclosure, or a pharmaceutically acceptable salt thereof, is co-administered with MK-8742+MK-5172. In certain embodiments, a compound of the present disclosure, or a pharmaceutically acceptable salt thereof, is co-administered with MK-3682. In certain embodiments, a compound of the present disclosure, or a pharmaceutically acceptable salt thereof, is co-administered with ACH-3422. In certain embodiments, a compound of the present disclosure, or a pharmaceutically acceptable salt thereof, is co-administered with AL-335. In certain embodiments, a compound of the present disclosure, or a pharmaceutically acceptable salt thereof, is co-administered with ACH-3422+ACH-3102. In certain embodiments, a compound of the present disclosure, or a pharmaceutically acceptable salt thereof, is co-administered with ACH-3422+sovaprevir+ACH-3102. In certain embodiments, a compound of the present disclosure, or a pharmaceutically acceptable salt thereof, is co-administered with GS-5816. In certain embodiments, a compound of the present disclosure, or a pharmaceutically acceptable salt thereof, is co-administered with GS-9857. In certain embodiments, a compound of the present disclosure, or a pharmaceutically acceptable salt thereof, is co-administered with IDX-21459. In certain embodiments, a compound of the present disclosure, or a pharmaceutically acceptable salt thereof, is co-administered with boceprevir. In certain embodiments, a compound of the present disclosure, or a pharmaceutically acceptable salt thereof, is co-administered with ledipasvir. In certain embodiments, a compound of the present disclosure, or a pharmaceutically acceptable salt thereof, is co-administered with AL-516.

In various methods, Compound A.1 is administered in an amount ranging from about 10 mg/day to about 200 mg/day. For example, the amount of Compound A.1 can be about 30 mg/day, about 45 mg/day, about 60 mg/day, about 90 mg/day, about 120 mg/day, about 135 mg/day, about 150 mg/day, about 180 mg/day. In some methods, Compound A.1 is administered at about 90 mg/day. In various methods, Compound A.2 is administered in an amount ranging from about 50 mg/day to about 800 mg/day. For example, the amount of Compound A.2 can be about 100 mg/day, about 200 mg/day, or about 400 mg/day. In some methods, the amount of Compound A.3 is about 10 mg/day to about 200 mg/day. For example, the amount of Compound A.3 can be about 25 mg/day, about 50 mg/day, about 75 mg/day, or about 100 mg/day.

In various methods, sofosbuvir is administered in an amount ranging from about 10 mg/day to about 1000 mg/day. For example, the amount of sofosbuvir can be about 100 mg/day, about 200 mg/day, about 300 mg/day, about 400 mg/day, about 500 mg/day, about 600 mg/day, about 700 mg/day, about 800 mg/day. In some methods, sofosbuvir is administered at about 400 mg/day.

Also provided herein is a compound of the present disclosure (e.g., a compound of Formula (I)), or a pharmaceutically acceptable salt thereof, and one or more additional therapeutic agents for treating HCV, for use in a method of treating or preventing HCV.

Also provided herein is a compound of the present disclosure (e.g., a compound of Formula (I)), or a pharmaceutically acceptable salt thereof, for use in a method of treating or preventing HCV, wherein the compound or a pharmaceutically acceptable salt thereof is administered simultaneously, separately or sequentially with one or more additional therapeutic agents for treating HCV.

### IX. Combination Therapy for HIV

In certain embodiments, a method for treating or preventing an HIV infection in a human having or at risk of having the infection is provided, comprising administering to the human a therapeutically effective amount of a compound of the present disclosure, or a pharmaceutically acceptable salt thereof, in combination with a therapeutically effective amount of one or more (e.g., one, two, three, one or two, or one to three) additional therapeutic agents. In one embodiment, a method for treating an HIV infection in a human having or at risk of having the infection is provided, comprising administering to the human a therapeutically effective amount of a compound of the present disclosure, or a pharmaceutically acceptable salt thereof, in combination with a therapeutically effective amount of one or more (e.g., one, two, three, one or two, or one to three) additional therapeutic agents.

In certain embodiments, the present disclosure provides a method for treating an HIV infection, comprising administering to a patient in need thereof a therapeutically effective amount of a compound of the present disclosure, or a pharmaceutically acceptable salt, thereof, in combination with a therapeutically effective amount of one or more additional therapeutic agents which are suitable for treating an HIV infection. In certain embodiments, one or more additional therapeutic agents includes, for example, one, two, three, four, one or two, one to three or one to four additional therapeutic agents.

In the above embodiments, the additional therapeutic agent may be an anti-HIV agent. For example, in some embodiments, the additional therapeutic agent is selected from the group consisting of HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors (e.g., CCR5 inhibitors, gp41 inhibitors (i.e., fusion inhibitors) and CD4 attachment inhibitors), CXCR4 inhibitors, gp120 inhibitors, G6PD and NADH-oxidase inhibitors, HIV vaccines, HIV maturation inhibitors, latency reversing agents (e.g., histone deacetylase inhibitors, proteasome inhibitors, protein kinase C (PKC) activators, and BRD4 inhibitors), compounds that target the HIV capsid (“capsid inhibitors”; e.g., capsid polymerization inhibitors or capsid disrupting compounds, HIV nucleocapsid p7 (NCp7) inhibitors, HIV p24 capsid protein inhibitors), pharmacokinetic enhancers, immune-based therapies (e.g., Pd-1 modulators, Pd-L1 modulators, toll like receptors modulators, IL-15 agonists), HIV antibodies, bispecific antibodies and “antibody-like” therapeutic proteins (e.g., DARTs®, Duobodies®, Bites®, XmAbs®, TandAbs®, Fab derivatives) including those targeting HIV gp120 or gp41, combination drugs for HIV, HIV p17 matrix protein inhibitors, IL-13 antagonists, Peptidyl-prolyl cis-trans isomerase A modulators, Protein disulfide isomerase inhibitors, Complement C5a receptor antagonists, DNA methyltransferase inhibitor, HIV vif gene modulators, HIV-1 viral infectivity factor inhibitors, TAT protein inhibitors, HIV-1 Nef modulators, Hck tyrosine kinase modulators, mixed lineage kinase-3 (MLK-3) inhibitors, HIV-1 splicing inhibitors, Rev protein inhibitors, Integrin antagonists, Nucleoprotein inhibitors, Splicing factor modulators, COMM domain containing protein 1 modulators, HIV Ribonuclease H inhibitors, Retrocyclin modulators, CDK-9 inhibitors, Dendritic ICAM-3 grabbing nonintegrin 1 inhibitors, HIV GAG protein inhibitors, HIV POL protein inhibitors, Complement Factor H modulators, Ubiquitin ligase inhibitors, Deoxycytidine kinase inhibitors, Cyclin dependent kinase inhibitors Proprotein convertase PC9 stimulators, ATP dependent RNA helicase DDX3X inhibitors, reverse transcriptase priming complex inhibitors, PI3K inhibitors, compounds such as those disclosed in WO 2013/006738 (Gilead Sciences), US 2013/0165489 (University of Pennsylvania), WO 2013/091096A1 (Boehringer Ingelheim), WO 2009/062285 (Boehringer Ingelheim), US20140221380 (Japan Tobacco), US20140221378 (Japan Tobacco), WO 2010/130034 (Boehringer Ingelheim), WO 2013/159064 (Gilead Sciences), WO 2012/145728 (Gilead Sciences), WO2012/003497 (Gilead Sciences), WO2014/100323 (Gilead Sciences), WO2012/145728 (Gilead Sciences), WO2013/159064 (Gilead Sciences) and WO 2012/003498 (Gilead Sciences) and WO 2013/006792 (Pharma Resources), and other drugs for treating HIV, and combinations thereof. In some embodiments, the additional therapeutic agent is further selected from Vif dimerization antagonists and HIV gene therapy.

In certain embodiments, the additional therapeutic is selected from the group consisting of HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, pharmacokinetic enhancers, and combinations thereof.

In certain embodiments a compound of the present disclosure is formulated as a tablet, which may optionally contain one or more other compounds useful for treating HIV. In certain embodiments, the tablet can contain another active ingredient for treating HIV, such as HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, pharmacokinetic enhancers, and combinations thereof.

In certain embodiments, such tablets are suitable for once daily dosing.

In certain embodiments, the additional therapeutic agent is selected from one or more of:

(1) Combination drugs selected from the group consisting of ATRIPLA® (efavirenz+tenofovir disoproxil fumarate+emtricitabine), COMPLERA® (EVIPLERA®, rilpivirine+tenofovir disoproxil fumarate+emtricitabine), STRIBILD® (elvitegravir+cobicistat+tenofovir disoproxil fumarate+emtricitabine), dolutegravir+abacavir sulfate+lamivudine, TRIUMEQ® (dolutegravir+abacavir+lamivudine), lamivudine+nevirapine+zidovudine, dolutegravir+rilpivirine, atazanavir sulfate+cobicistat, darunavir+cobicistat, efavirenz+lamivudine+tenofovir disoproxil fumarate, tenofovir alafenamide hemifumarate+emtricitabine+cobicistat+elvitegravir, Vacc-4x+romidepsin, darunavir+tenofovir alafenamide hemifumarate+emtricitabine+cobicistat, APH-0812, raltegravir+lamivudine, KALETRA® (ALUVIA®, lopinavir+ritonavir), atazanavir sulfate+ritonavir, COMBIVIR® (zidovudine+lamivudine, AZT+3TC), EPZICOM® (Livexa®, abacavir sulfate+lamivudine, ABC+3TC), TRIZIVIR® (abacavir sulfate+zidovudine+lamivudine, ABC+AZT+3TC), TRUVADA® (tenofovir disoproxil fumarate+emtricitabine, TDF+FTC), tenofovir+lamivudine and lamivudine+tenofovir disoproxil fumarate, as well as combinations drugs selected from dolutegravir+rilpivirine hydrochloride, atazanavir+cobicistat, tenofovir alafenamide hemifumarate+emtricitabine, tenofovir alafenamide+emtricitabine, tenofovir alafenamide hemifumarate+emtricitabine+rilpivirine, tenofovir alafenamide+emtricitabine+rilpivirine, doravirine+lamivudine+tenofovir disoproxil fumarate, doravirine+lamivudine+tenofovir disoproxil;
(2) HIV protease inhibitors selected from the group consisting of amprenavir, atazanavir, fosamprenavir, fosamprenavir calcium, indinavir, indinavir sulfate, lopinavir, ritonavir, nelfinavir, nelfinavir mesylate, saquinavir, saquinavir mesylate, tipranavir, brecanavir, darunavir, DG-17, TMB-657 (PPL-100) and TMC-310911;
(3) HIV non-nucleoside or non-nucleotide inhibitors of reverse transcriptase selected from the group consisting of delavirdine, delavirdine mesylate, nevirapine, etravirine, dapivirine, doravirine, rilpivirine, efavirenz, KM-023, VM-1500, lentinan and AIC-292;
(4) HIV nucleoside or nucleotide inhibitors of reverse transcriptase selected from the group consisting of VIDEX® and VIDEX® EC (didanosine, ddl), zidovudine, emtricitabine, didanosine, stavudine, zalcitabine, lamivudine, censavudine, abacavir, abacavir sulfate, amdoxovir, elvucitabine, alovudine, phosphazid, fozivudine tidoxil, apricitabine, amdoxovir, KP-1461, fosalvudine tidoxil, tenofovir, tenofovir disoproxil, tenofovir disoproxil fumarate, tenofovir disoproxil hemifumarate, tenofovir alafenamide, tenofovir alafenamide hemifumarate, tenofovir alafenamide fumarate, adefovir, adefovir dipivoxil, and festinavir;
(5) HIV integrase inhibitors selected from the group consisting of curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, raltegravir, elvitegravir, dolutegravir and cabotegravir, as well as HIV integrase inhibitors selected from JTK-351;
(6) HIV non-catalytic site, or allosteric, integrase inhibitors (NCINI) selected from the group consisting of CX-05168, CX-05045 and CX-14442;
(7) HIV gp41 inhibitors selected from the group consisting of enfuvirtide, sifuvirtide and albuvirtide;
(8) HIV entry inhibitors selected from the group consisting of cenicriviroc;
(9) HIV gp120 inhibitors selected from the group consisting of Radha-108 (Receptol) and BMS-663068;
(10) CCR5 inhibitors selected from the group consisting of aplaviroc, vicriviroc, maraviroc, cenicriviroc, PRO-140, Adaptavir (RAP-101), TBR-220 (TAK-220), nifeviroc (TD-0232), TD-0680, and vMIP (Haimipu);
(11) CD4 attachment inhibitors selected from the group consisting of ibalizumab;
(12) CXCR4 inhibitors selected from the group consisting of plerixafor, ALT-1188, vMIP and Haimipu;
(13) Pharmacokinetic enhancers selected from the group consisting of cobicistat and ritonavir;
(14) Immune-based therapies selected from the group consisting of dermaVir, interleukin-7, plaquenil (hydroxychloroquine), proleukin (aldesleukin, IL-2), interferon alfa, interferon alfa-2b, interferon alfa-n3, pegylated interferon alfa, interferon gamma, hydroxyurea, mycophenolate mofetil (MPA) and its ester derivative mycophenolate mofetil (MMF), WF-10, ribavirin, IL-2, IL-12, polymer polyethyleneimine (PEI), Gepon, VGV-1, MOR-22, BMS-936559, toll-like receptors modulators (TLR-1, TLR-2, TLR-3, TLR-4, TLR-5, TLR-6, TLR-7, TLR-8, TLR-9, TLR-10, TLR-11, TLR-12 and TLR-13), rintatolimod and IR-103;
(15) HIV vaccines selected from the group consisting of peptide vaccines, recombinant subunit protein vaccines, live vector vaccines, DNA vaccines, virus-like particle vaccines (pseudovirion vaccine), CD4-derived peptide vaccines, vaccine combinations, rgp120 (AIDSVAX), ALVAC HIV (vCP1521)/AIDSVAX B/E (gp120) (RV144), Remune, ITV-1, Contre Vir, Ad5-ENVA-48, DCVax-001 (CDX-2401), PEP-6409, Vacc-4x, Vacc-C5, VAC-3S, multiclade DNA recombinant adenovirus-5 (rAd5), Pennvax-G, VRC-HIV MAB060-00-AB, AVX-101, Tat Oyi vaccine, AVX-201, HIV-LAMP-vax, Ad35, Ad35-GRIN, NAcGM3/VSSP ISA-51, poly-ICLC adjuvanted vaccines, TatImmune, GTU-multiHIV (FIT-06), AGS-004, gp140[delta]V2.TV1+MF-59, rVSVIN HIV-1 gag vaccine, SeV-Gag vaccine, AT-20, DNK-4, Ad35-GRIN/ENV, TBC-M4, HIVAX, HIVAX-2, NYVAC-HIV-PT1, NYVAC-HIV-PT4, DNA-HIV-PT123, Vichrepol, rAAV1-PG9DP, GOVX-B11, GOVX-B21, ThV-01, TUTI-16, VGX-3300, TVI-HIV-1, Ad-4 (Ad4-env Clade C+Ad4-mGag), EN41-UGR7C, EN41-FPA2, PreVaxTat, TL-01, SAV-001, AE-H, MYM-V101, CombiHIVvac, ADVAX, MYM-V201, MVA-CMDR, ETV-01 and DNA-Ad5 gag/pol/nef/nev (HVTN505), as well as HIV vaccines selected from monomeric gp120 HIV-1 subtype C vaccine (Novartis), HIV-TriMix-mRNA, MVATG-17401, ETV-01, CDX-1401, and rcAd26.MOS1.HIV-Env;
(16) HIV antibodies, bispecific antibodies and “antibody-like” therapeutic proteins (such as DARTs®, Duobodies®, Bites®, XmAbs®, TandAbs®, Fab derivatives) including BMS-936559, TMB-360 and those targeting HIV gp120 or gp41 selected from the group consisting of bavituximab, UB-421, C2F5, C2G12, C4E10, C2F5+C2G12+C4E10, 3-BNC-117, PGT145, PGT121, MDX010 (ipilimumab), VRC01, A32, 7B2, 10E8 and VRC07, as well as HIV antibodies such as VRC-07-523;
(17) latency reversing agents selected from the group consisting of Histone deacetylase inhibitors such as Romidepsin, vorinostat, panobinostat; Proteasome inhibitors such as Velcade; protein kinase C (PKC) activators such as Indolactam, Prostratin, Ingenol B and DAG-lactones, Ionomycin, GSK-343, PMA, SAHA, BRD4 inhibitors, IL-15, JQ1, disulfiram, and amphotericin B;
(18) HIV nucleocapsid p7 (NCp7) inhibitors selected from the group consisting of azodicarbonamide;
(19) HIV maturation inhibitors selected from the group consisting of BMS-955176 and GSK-2838232;
(20) PI3K inhibitors selected from the group consisting of idelalisib, AZD-8186, buparlisib, CLR-457, pictilisib, neratinib, rigosertib, rigosertib sodium, EN-3342, TGR-1202, alpelisib, duvelisib, UCB-5857, taselisib, XL-765, gedatolisib, VS-5584, copanlisib, CAI orotate, perifosine, RG-7666, GSK-2636771, DS-7423, panulisib, GSK-2269557, GSK-2126458, CUDC-907, PQR-309, INCB-040093, pilaralisib, BAY-1082439, puquitinib mesylate, SAR-245409, AMG-319, RP-6530, ZSTK-474, MLN-1117, SF-1126, RV-1729, sonolisib, LY-3023414, SAR-260301 and CLR-1401;
(21) the compounds disclosed in WO 2004/096286 (Gilead Sciences), WO 2006/110157 (Gilead Sciences), WO 2006/015261 (Gilead Sciences), WO 2013/006738 (Gilead Sciences), US 2013/0165489 (University of Pennsylvania), US20140221380 (Japan Tobacco), US20140221378 (Japan Tobacco), WO 2013/006792 (Pharma Resources), WO 2009/062285 (Boehringer Ingelheim), WO 2010/130034 (Boehringer Ingelheim), WO 2013/091096A1 (Boehringer Ingelheim), WO 2013/159064 (Gilead Sciences), WO 2012/145728 (Gilead Sciences), WO2012/003497 (Gilead Sciences), WO2014/100323 (Gilead Sciences), WO2012/145728 (Gilead Sciences), WO2013/159064 (Gilead Sciences) and WO 2012/003498 (Gilead Sciences); and
(22) other drugs for treating HIV selected from the group consisting of BanLec, MK-8507, AG-1105, TR-452, MK-8591, REP 9, CYT-107, alisporivir, NOV-205, IND-02, metenkefalin, PGN-007, Acemannan, Gamimune, Prolastin, 1,5-dicaffeoylquinic acid, BIT-225, RPI-MN, VSSP, Hlviral, IMO-3100, SB-728-T, RPI-MN, VIR-576, HGTV-43, MK-1376, rHIV7-shl-TAR-CCR5RZ, MazF gene therapy, BlockAide, ABX-464, SCY-635, naltrexone and PA-1050040 (PA-040); and other drugs for treating HIV selected from AAV-eCD4-Ig gene therapy, TEV-90110, TEV-90112, TEV-90111, TEV-90113, deferiprone, and HS-10234.

In certain embodiments, the additional therapeutic agent is a compound disclosed in US 2014-0221356 (Gilead Sciences, Inc.) for example (2R,5S,13aR)—N-(2,4-difluorobenzyl)-8-hydroxy-7,9-dioxo-2,3,4,5,7,9,13,13a-octahydro-2,5-methanopyrido[1′,2′:4,5]pyrazino[2,1-b][1,3]oxazepine-10-carboxamide, (2S,5R,13aS)—N-(2,4-difluorobenzyl)-8-hydroxy-7,9-dioxo-2,3,4,5,7,9,13,13a-octahydro-2,5-methanopyrido[1′,2′:4,5]pyrazino[2,1-b][1,3]oxazepine-10-carboxamide, (1S,4R,12aR)—N-(2,4-difluorobenzyl)-7-hydroxy-6,8-dioxo-1,2,3,4,6,8,12,12a-octahydro-1,4-methanodipyrido[1,2-a:1′,2′-d]pyrazine-9-carboxamide, (1R,4S,12aR)-7-hydroxy-6,8-dioxo-N-(2,4,6-trifluorobenzyl)-1,2,3,4,6,8,12,12a-octahydro-1,4-methanodipyrido[1,2-a:1′,2′-d]pyrazine-9-carboxamide, (2R,5S,13aR)-8-hydroxy-7,9-dioxo-N-(2,4,6-trifluorobenzyl)-2,3,4,5,7,9,13,13a-octahydro-2,5-methanopyrido[1′,2′:4,5]pyrazino[2,1-b][1,3]oxazepine-10-carboxamide, and (1R,4S,12aR)—N-(2,4-difluorobenzyl)-7-hydroxy-6,8-dioxo-1,2,3,4,6,8,12,12a-octahydro-1,4-methanodipyrido[1,2-a:1′,2′-d]pyrazine-9-carboxamide, US2015-0018298 (Gilead Sciences, Inc.) and US2015-0018359 (Gilead Sciences, Inc.),

In certain embodiments, a compound of the present disclosure, or a pharmaceutically acceptable salt thereof, is combined with one, two, three, four or more additional therapeutic agents. In certain embodiments, a compound of the present disclosure, or a pharmaceutically acceptable salt thereof, is combined with two additional therapeutic agents. In other embodiments, a compound of the present disclosure, or a pharmaceutically acceptable salt thereof, is combined with three additional therapeutic agents. In further embodiments, a compound of the present disclosure, or a

pharmaceutically acceptable salt thereof, is combined with four additional therapeutic agents. The one, two, three, four or more additional therapeutic agents can be different therapeutic agents selected from the same class of therapeutic agents, and/or they can be selected from different classes of therapeutic agents.

In a specific embodiment, a compound of the present disclosure, or a pharmaceutically acceptable salt thereof, is combined with an HIV nucleoside or nucleotide inhibitor of reverse transcriptase and an HIV non-nucleoside inhibitor of reverse transcriptase. In another specific embodiment, a compound of the present disclosure, or a pharmaceutically acceptable salt thereof, is combined with an HIV nucleoside or nucleotide inhibitor of reverse transcriptase, and an HIV protease inhibiting compound. In a further embodiment, a compound of the present disclosure, or a pharmaceutically acceptable salt thereof, is combined with an HIV nucleoside or nucleotide inhibitor of reverse transcriptase, an HIV non-nucleoside inhibitor of reverse transcriptase, and an HIV protease inhibiting compound. In an additional embodiment, a compound of the present disclosure, or a pharmaceutically acceptable salt thereof, is combined with an HIV nucleoside or nucleotide inhibitor of reverse transcriptase, an HIV non-nucleoside inhibitor of reverse transcriptase, and a pharmacokinetic enhancer. In certain embodiments, a compound of the present disclosure, or a pharmaceutically acceptable salt thereof, is combined with one or more additional therapeutic agents selected from HIV nucleoside inhibitor of reverse transcriptase, an integrase inhibitor, and a pharmacokinetic enhancer. In another embodiment, a compound of the present disclosure, or a pharmaceutically acceptable salt thereof, is combined with two HIV nucleoside or nucleotide inhibitors of reverse transcriptase.

In a particular embodiment, a compound of the present disclosure, or a pharmaceutically acceptable salt thereof, is combined with one, two, three, four or more additional therapeutic agents selected from Triumeq® (dolutegravir+abacavir+lamivudine), dolutegravir+abacavir sulfate+lamivudine, raltegravir, Truvada® (tenofovir disoproxil fumarate+emtricitabine, TDF+FTC), maraviroc, enfuvirtide, Epzicom® (Livexa®, abacavir sulfate+lamivudine, ABC+3TC), Trizivir® (abacavir sulfate+zidovudine+lamivudine, ABC+AZT+3TC), adefovir, adefovir dipivoxil, Stribild® (elvitegravir+cobicistat+tenofovir disoproxil fumarate+emtricitabine), rilpivirine, rilpivirine hydrochloride, Complera® (Eviplera®, rilpivirine+tenofovir disoproxil fumarate+emtricitabine), Cobicistat, Atripla® (efavirenz+tenofovir disoproxil fumarate+emtricitabine), atazanavir, atazanavir sulfate, dolutegravir, elvitegravir, Aluvia® (Kaletra®, lopinavir+ritonavir), ritonavir, emtricitabine, atazanavir sulfate+ritonavir, darunavir, lamivudine, Prolastin, fosamprenavir, fosamprenavir calcium, efavirenz, Combivir® (zidovudine+lamivudine, AZT+3TC), etravirine, nelfinavir, nelfinavir mesylate, interferon, didanosine, stavudine, indinavir, indinavir sulfate, tenofovir+lamivudine, zidovudine, nevirapine, saquinavir, saquinavir mesylate, aldesleukin, zalcitabine, tipranavir, amprenavir, delavirdine, delavirdine mesylate, Radha-108 (Receptol), Hlviral, lamivudine+tenofovir disoproxil fumarate, efavirenz+lamivudine+tenofovir disoproxil fumarate, phosphazid, lamivudine+nevirapine+zidovudine, abacavir, abacavir sulfate, tenofovir, tenofovir disoproxil, tenofovir disoproxil fumarate, tenofovir alafenamide and tenofovir alafenamide hemifumarate. In certain embodiments, the one, two, three, four or more additional therapeutic agents are further selected from raltegravir+lamivudine, atazanavir sulfate+cobicistat, atazanavir+cobicistat, darunavir+cobicistat, darunavir+cobicistat, atazanavir sulfate+cobicistat, atazanavir+cobicistat.

In a particular embodiment, a compound of the present disclosure, or a pharmaceutically acceptable salt thereof, is combined with one, two, three, four or more additional therapeutic agents selected from Triumeq® (dolutegravir+abacavir+lamivudine), dolutegravir+abacavir sulfate+lamivudine, raltegravir, Truvada® (tenofovir disoproxil fumarate+emtricitabine, TDF+FTC), maraviroc, enfuvirtide, Epzicom® (Livexa®, abacavir sulfate+lamivudine, ABC+3TC), Trizivir® (abacavir sulfate+zidovudine+lamivudine, ABC+AZT+3TC), adefovir, adefovir dipivoxil, Stribild® (elvitegravir+cobicistat+tenofovir disoproxil fumarate+emtricitabine), rilpivirine, rilpivirine hydrochloride, Complera® (Eviplera®, rilpivirine+tenofovir disoproxil fumarate+emtricitabine), cobicistat, Atripla® (efavirenz+tenofovir disoproxil fumarate+emtricitabine), atazanavir, atazanavir sulfate, dolutegravir, elvitegravir, Aluvia® (Kaletra®, lopinavir+ritonavir), ritonavir, emtricitabine, atazanavir sulfate+ritonavir, darunavir, lamivudine, Prolastin, fosamprenavir, fosamprenavir calcium, efavirenz, Combivir® (zidovudine+lamivudine, AZT+3TC), etravirine, nelfinavir, nelfinavir mesylate, interferon, didanosine, stavudine, indinavir, indinavir sulfate, tenofovir+lamivudine, zidovudine, nevirapine, saquinavir, saquinavir mesylate, aldesleukin, zalcitabine, tipranavir, amprenavir, delavirdine, delavirdine mesylate, Radha-108 (Receptol), Hlviral, lamivudine+tenofovir disoproxil fumarate, efavirenz+lamivudine+tenofovir disoproxil fumarate, phosphazid, lamivudine+nevirapine+zidovudine, (2R,5S,13aR)—N-(2,4-difluorobenzyl)-8-hydroxy-7,9-dioxo-2,3,4,5,7,9,13,13a-octahydro-2,5-methanopyrido[1′,2′:4,5]pyrazino[2,1-b][1,3]oxazepine-10-carboxamide, (2S,5R,13aS)—N-(2,4-difluorobenzyl)-8-hydroxy-7,9-dioxo-2,3,4,5,7,9,13,13a-octahydro-2,5-methanopyrido[1′,2′:4,5]pyrazino[2,1-b][1,3]oxazepine-10-carboxamide, (1S,4R,12aR)—N-(2,4-difluorobenzyl)-7-hydroxy-6,8-dioxo-1,2,3,4,6,8,12,12a-octahydro-1,4-methanodipyrido[1,2-a:1′,2′-d]pyrazine-9-carboxamide, (1R,4S,12aR)-7-hydroxy-6,8-dioxo-N-(2,4,6-trifluorobenzyl)-1,2,3,4,6,8,12,12a-octahydro-1,4-methanodipyrido[1,2-a:1′,2′-d]pyrazine-9-carboxamide, (2R,5S,13aR)-8-hydroxy-7,9-dioxo-N-(2,4,6-trifluorobenzyl)-2,3,4,5,7,9,13,13a-octahydro-2,5-methanopyrido[1′,2′:4,5]pyrazino[2,1-b][1,3]oxazepine-10-carboxamide, and (1R,4S,12aR)—N-(2,4-difluorobenzyl)-7-hydroxy-6,8-dioxo-1,2,3,4,6,8,12,12a-octahydro-1,4-methanodipyrido[1,2-a:1′,2′-d]pyrazine-9-carboxamide abacavir, abacavir sulfate, tenofovir, tenofovir disoproxil, tenofovir disoproxil fumarate, tenofovir alafenamide and tenofovir alafenamide hemifumarate.

In a particular embodiment, a compound of the present disclosure, or a pharmaceutically acceptable salt thereof, is combined with abacavir sulfate, tenofovir, tenofovir disoproxil, tenofovir disoproxil fumarate, tenofovir disoproxil hemifumarate, tenofovir alafenamide or tenofovir alafenamide hemifumarate.

In a particular embodiment, a compound of the present disclosure, or a pharmaceutically acceptable salt thereof, is combined with tenofovir, tenofovir disoproxil, tenofovir disoproxil fumarate, tenofovir alafenamide, or tenofovir alafenamide hemifumarate.

In a particular embodiment, a compound of the present disclosure, or a pharmaceutically acceptable salt thereof, is combined with a first additional therapeutic agent selected from the group consisting of: abacavir sulfate, tenofovir, tenofovir disoproxil, tenofovir disoproxil fumarate, tenofovir alafenamide, and tenofovir alafenamide hemifumarate and a second additional therapeutic agent selected from the group consisting of emtricitabine and lamivudine.

In a particular embodiment, a compound of the present disclosure, or a pharmaceutically acceptable salt thereof, is combined with a first additional therapeutic agent selected from the group consisting of: tenofovir, tenofovir disoproxil, tenofovir disoproxil fumarate, tenofovir alafenamide, and tenofovir alafenamide hemifumarate and a second additional therapeutic agent, wherein the second additional therapeutic agent is emtricitabine.

In certain embodiments, a compound of the present disclosure, or a pharmaceutically acceptable salt thereof, is combined with 5-30 mg tenofovir alafenamide fumarate, tenofovir alafenamide hemifumarate, or tenofovir alafenamide and 200 mg emtricitabine. In certain embodiments, a compound of the present disclosure, or a pharmaceutically acceptable salt thereof, is combined with 5-10; 5-15; 5-20; 5-25; 25-30; 20-30; 15-30; or 10-30 mg tenofovir alafenamide fumarate, tenofovir alafenamide hemifumarate, or tenofovir alafenamide and 200 mg emtricitabine. In certain embodiments, a compound of the present disclosure, or a pharmaceutically acceptable salt thereof, is combined with 10 mg tenofovir alafenamide fumarate, tenofovir alafenamide hemifumarate, or tenofovir alafenamide and 200 mg emtricitabine. In certain embodiments, a compound of the present disclosure, or a pharmaceutically acceptable salt thereof, is combined with 25 mg tenofovir alafenamide fumarate, tenofovir alafenamide hemifumarate, or tenofovir alafenamide and 200 mg emtricitabine. A compound of the present disclosure (e.g., a compound of formula (I)) may be combined with the agents provided herein in any dosage amount of the compound (e.g., from 1 mg to 500 mg of compound) the same as if each combination of dosages were specifically and individually listed.

In certain embodiments, a compound of the present disclosure, or a pharmaceutically acceptable salt thereof, is combined with 200-400 mg tenofovir disoproxil fumarate, tenofovir disoproxil hemifumarate, or tenofovir disoproxil and 200 mg emtricitabine. In certain embodiments, a compound of the present disclosure, or a pharmaceutically acceptable salt thereof, is combined with 200-250; 200-300; 200-350; 250-350; 250-400; 350-400; 300-400; or 250-400 mg tenofovir disoproxil fumarate, tenofovir disoproxil hemifumarate, or tenofovir disoproxil and 200 mg emtricitabine. In certain embodiments, a compound of the present disclosure, or a pharmaceutically acceptable salt thereof, is combined with 300 mg tenofovir disoproxil fumarate, tenofovir disoproxil hemifumarate, or tenofovir disoproxil and 200 mg emtricitabine. A compound of the present disclosure (e.g., a compound of formula (I)) may be combined with the agents provided herein in any dosage amount of the compound (e.g., from 50 mg to 500 mg of compound) the same as if each combination of dosages were specifically and individually listed. A compound of the present disclosure (e.g., a compound of Formula (I)) may be combined with the agents provided herein in any dosage amount of the compound (e.g. from about 1 mg to about 150 mg of compound) the same as if each combination of dosages were specifically and individually listed.

In certain embodiments a compound of the present disclosure, or a pharmaceutically acceptable salt thereof, is combined with (2R,5S,13aR)—N-(2,4-difluorobenzyl)-8-hydroxy-7,9-dioxo-2,3,4,5,7,9,13,13a-octahydro-2,5-methanopyrido[1′,2′:4,5]pyrazino[2,1-b][1,3]oxazepine-10-carboxamide, (2S,5R,13aS)—N-(2,4-difluorobenzyl)-8-hydroxy-7,9-dioxo-2,3,4,5,7,9,13,13a-octahydro-2,5-methanopyrido[1′,2′:4,5]pyrazino[2,1-b][1,3]oxazepine-10-carboxamide, (1S,4R,12aR)—N-(2,4-difluorobenzyl)-7-hydroxy-6,8-dioxo-1,2,3,4,6,8,12,12a-octahydro-1,4-methanodipyrido[1,2-a:1′,2′-d]pyrazine-9-carboxamide, (1R,4S,12aR)-7-hydroxy-6,8-dioxo-N-(2,4,6-trifluorobenzyl)-1,2,3,4,6,8,12,12a-octahydro-1,4-methanodipyrido[1,2-a:1′,2′-d]pyrazine-9-carboxamide, (2R,5S,13aR)-8-hydroxy-7,9-dioxo-N-(2,4,6-trifluorobenzyl)-2,3,4,5,7,9,13,13a-octahydro-2,5-methanopyrido[1′,2′:4,5]pyrazino[2,1-b][1,3]oxazepine-10-carboxamide, or (1R,4S,12aR)—N-(2,4-difluorobenzyl)-7-hydroxy-6,8-dioxo-1,2,3,4,6,8,12,12a-octahydro-1,4-methanodipyrido[1,2-a:1′,2′-d]pyrazine-9-carboxamide.

Also provided herein is a compound the present disclosure (e.g., a compound of Formula (I)), or a pharmaceutically acceptable salt thereof, and one or more additional therapeutic agents for treating HIV, for use in a method of treating or preventing HIV.

Also provided herein is a compound of the present disclosure (e.g., a compound of Formula (I)), or a pharmaceutically acceptable salt thereof, for use in a method of treating or preventing HIV, wherein the compound or a pharmaceutically acceptable salt thereof is administered simultaneously, separately or sequentially with one or more additional therapeutic agents for treating HIV.

In certain embodiments, a method for treating hyperproliferative disorders such as cancer in a human is provided, comprising administering to the human a therapeutically effective amount of a compound of the present disclosure, or a pharmaceutically acceptable salt thereof, in combination with a therapeutically effective amount of one or more (e.g., one, two, three, one or two, or one to three) additional therapeutic agents. In one embodiment, a method for treating hyperproliferative disorders such as cancer in a human is provided, comprising administering to the human a therapeutically effective amount of a compound of the present disclosure, or a pharmaceutically acceptable salt thereof, in combination with a therapeutically effective amount of one or more (e.g., one, two, three, one or two, or one to three) additional therapeutic agents.

### X. Combination Therapy for Cancer

In certain embodiments, the present disclosure provides a method for treating hyperproliferative disorders such as cancer, comprising administering to a patient in need thereof a therapeutically effective amount of a compound of the present disclosure, or a pharmaceutically acceptable salt, thereof, in combination with a therapeutically effective amount of one or more additional therapeutic agents which are suitable for treating hyperproliferative disorders such as cancer.

In the above embodiments, the additional therapeutic agent may be an anti-cancer agent. For example, in some embodiments, the additional therapeutic agent is selected from the group consisting of chemotherapeutic agents, immunotherapeutic agents, radiotherapeutic agents, anti-neoplastic agents, anti-hormonal agents, anti-angiogenic agents, anti-fibrotic agents, therapeutic antibodies, tyrosine kinase inhibitors, JAK inhibitors, Hedgehog inhibitors, HDAC inhibitors, Discoidin domain receptor (DDR) inhibitors, MMP9 inhibitors, LOXL inhibitors, ASK1 inhibitors, PI3K inhibitors, BTK inhibitors, SYK inhibitors, mTOR inhibitors, AKT inhibitors, Mitogen or Extracellular Regulated Kinase (MEK) inhibitors, blockers of Raf kinases (rafk), CDK inhibitors, JNK inhibitors, MAPK inhibitors, Raf inhibitors, ROCK inhibitors, Tie2 inhibitors, Myo-inositol signaling inhibitors, phospholipase C blockers, anti-CD19 antibodies, anti-CD20 antibodies, anti-MN-14 antibodies, Anti-TRAIL DR4 and DR5 antibodies, anti-CD74 antibodies, cancer vaccines based upon the genetic makeup of an individual patient's tumor, IDH1 inhibitors, BRD4 inhibitors, TPL2 inhibitors; A2B inhibitors; TBK1 inhibitors; IKK inhibitors; BCR inhibitors, agents inhibiting the RAS/RAF/ERK pathway, protein kinase C (PKC) modulators, modulators of growth factor receptors such as epidermal growth factor receptor (EGFr), platelet derived growth factor receptor (PDGFr), erbB2, erbB4, ret, vascular endothelial growth factor receptor (VEGFr), tyrosine kinase with immunoglobulin-like and epidermal growth factor homology domains (TIE-2), insulin growth factor-I (IGFI) receptor, macrophage colony stimulating factor (cfms), BTK, ckit, cmet, fibroblast growth factor (FGF) receptors, Trk receptors (TrkA, TrkB, and TrkC), ephrin (eph) receptors, and the RET protooncogene, modulators of tyrosine kinases including cSrc, Lck, Fyn, Yes, cAbl, FAK (Focal adhesion kinase) and Bcr-Abl, modulators of PKB family kinases, modulators of TGF beta receptor kinases, inhibitors of Ras oncogene including inhibitors of farnesyltransferase, geranyl-geranyl transferase, and CAAX proteases, anti-sense oligonucleotides, ribozymes, Bcl-2 family protein inhibitors, proteasome inhibitors, Heat shock protein HSP90 inhibitors, combination drugs and immunotherapy, and other drugs for treating hyperproliferative disorders such as cancer, and combinations thereof.

In certain embodiments a compound of the present disclosure is formulated as a tablet, which may optionally contain one or more other compounds useful for treating cancer. In certain embodiments, the tablet can contain another active ingredient for treating cancer, such as chemotherapeutic agents, immunotherapeutic agents, radiotherapeutic agents, anti-neoplastic agents, anti-fibrotic agents, anti-hormonal agents, anti-angiogenic agents, Tyrosine kinase inhibitors, JAK inhibitors, Hedgehog inhibitors, HDAC inhibitors, Discoidin domain receptor (DDR) inhibitors, MMP9 inhibitors, LOXL inhibitors, ASK1 inhibitors, PI3K inhibitors, BTK inhibitors, SYK inhibitors, mTOR inhibitors, AKT inhibitors, Mitogen or Extracellular Regulated Kinase (MEK) inhibitors, blockers of Raf kinases (rafk), CDK inhibitors, JNK inhibitors, MAPK inhibitors, Raf inhibitors, ROCK inhibitors, Tie2 inhibitors, Myo-inositol signaling inhibitors, phospholipase C blockers, IDH1 inhibitors, BRD4 inhibitors, TPL2 inhibitors; A2B inhibitors; TBK1 inhibitors; IKK inhibitors; BCR inhibitors, agents inhibiting the RAS/RAF/ERK pathway, protein kinase C (PKC) modulators, modulators of growth factor receptors such as epidermal growth factor receptor (EGFr), platelet derived growth factor receptor (PDGFr), erbB2, erbB4, ret, vascular endothelial growth factor receptor (VEGFr), tyrosine kinase with immunoglobulin-like and epidermal growth factor homology domains (TIE-2), insulin growth factor-I (IGFI) receptor, macrophage colony stimulating factor (cfms), BTK, ckit, cmet, fibroblast growth factor (FGF) receptors, Trk receptors (TrkA, TrkB, and TrkC), ephrin (eph) receptors, and the RET protooncogene, modulators of tyrosine kinases including cSrc, Lck, Fyn, Yes, cAbl, FAK (Focal adhesion kinase) and Bcr-Abl, modulators of PKB family kinases, modulators of TGF beta receptor kinases, inhibitors of Ras oncogene including inhibitors of farnesyltransferase, geranyl-geranyl transferase, and CAAX proteases, anti-sense oligonucleotides, ribozymes, Bcl-2 family protein inhibitors, proteasome inhibitors, Heat shock protein HSP90 inhibitors, combination drugs and immunotherapy, and other drugs for treating hyperproliferative disorders such as cancer, and combinations thereof.

In certain embodiments, such tablets are suitable for once daily dosing. In certain embodiments, the additional therapeutic agent is selected from one or more of: (1) Chemotherapeutic agents selected from the group consisting of: anti-metabolites/anti-cancer agents, such as pyrimidine analogs (floxuridine, capecitabine, and cytarabine); purine analogs, folate antagonists and related inhibitors, antiproliferative/antimitotic agents including natural products such as vinca alkaloid (vinblastine, vincristine) and microtubule such as taxane (paclitaxel, docetaxel), vinblastin, nocodazole, epothilones and navelbine, epipodophyllotoxins (etoposide, teniposide); DNA damaging agents (actinomycin, amsacrine, busulfan, carboplatin, chlorambucil, cisplatin, cyclophosphamide, Cytoxan, dactinomycin, daunorubicin, doxorubicin, epirubicin, iphosphamide, melphalan, mechlorethamine, mitomycin, mitoxantrone, nitrosourea, procarbazine, taxol, taxotere, teniposide, etoposide, triethylenethiophosphoramide); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards cyclophosphamide and analogs, melphalan, chlorambucil), and (hexamethylmelamine and thiotepa), alkyl nitrosoureas (BCNU) and analogs, streptozocin, trazenes-dacarbazinine (DTIC); antiproliferative/antimitotic antimetabolites such as folic acid analogs (methotrexate); platinum coordination complexes (cisplatin, oxiloplatinim, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel; antimigratory agents; antisecretory agents (breveldin); immunosuppressives tacrolimus, sirolimus azathioprine, mycophenolate; compounds (TNP-470, genistein) and growth factor inhibitors (vascular endothelial growth factor inhibitors, fibroblast growth factor inhibitors); angiotensin receptor blocker, nitric oxide donors; anti-sense oligonucleotides; cell cycle inhibitors and differentiation inducers (tretinoin); inhibitors, topoisomerase inhibitors (doxorubicin (adriamycin), daunorubicin, dactinomycin, eniposide, epirubicin, idarubicin, irinotecan and mitoxantrone, topotecan, irinotecan), corticosteroids (cortisone, dexamethasone, hydrocortisone, methylprednisolone, prednisone, and prednisolone); growth factor signal transduction kinase inhibitors; dysfunction inducers, toxins such as Cholera toxin, ricin, Pseudomonas exotoxin, Bordetella pertussis adenylate cyclase toxin, or diphtheria toxin, and caspase activators, chromatin, alkylating agents such as thiotepa and cyclophosphamide (Cytoxan, Endoxan, Endoxana, Cyclostin), alkyl sulfonates such as busulfan, improsulfan and piposulfan; aziridines such as benzodopa, carboquone, meturedopa, and uredopa; emylerumines and memylamelamines including alfretamine, triemylenemelamine, triethylenephosphoramide, triethylenethiophosphoramide and trimemylolomelamine; acetogenins (especially bullatacin and bullatacinone); a camptothecin (including synthetic analogue topotecan); bryostatin; callystatin; CC-1065 (including its adozelesin, carzelesin and bizelesin synthetic analogues); cryptophycins (articularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, foremustine, lomustine, nimustine, ranimustine; antibiotics such as the enediyne antibiotics (e.g., calicheamicin, especially calicheamicin gammaII and calicheamicin phiI1, see, e.g., Agnew, Chem. Intl. Ed. Engl, 33:183-186 (1994); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin, PEGylated liposomal doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as demopterin, methotrexate, pteropterin, trimetrexate; purine analogs such as fludarabine, 6-mercaptopurine, thiamiprine, thioguanine; pyrimidine analogues such as ancitabine, azacitidine, 6-azauridine, carmofur, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replinisher such as frolinic acid; aceglatone; aldophosphamide glycoside; aminolevulinic acid; eniluracil; amsacrine; hestrabucil; bisantrene; edatraxate; defofamine; demecolcine; diaziquone; elformthine;

elliptinium acetate; an epothilone; etoglucid; gallium nitrate; hydroxyurea; lentinan; leucovorin; lonidamine; maytansinoids such as maytansine and ansamitocins; mitoguazone; mitoxantrone; mopidamol; nitracrine; pentostatin; phenamet; pirarubicin; losoxantrone; fluoropyrimidine; folinic acid; podophyllinic acid; 2-ethylhydrazide; procarbazine; PSK(r); razoxane; rhizoxin; sizofiran; spirogermanium; tenuazonic acid; triaziquone; 2,2′,2″-tricUorotriemylamine; trichothecenes (especially T-2 toxin, verracurin A, roridin A and anguidine); urethane; vindesine; dacarbazine; mannomustine; mitobronitol; mitolactol; pipobroman; gacytosine; arabinoside (“Ara-C”); cyclophosphamide; thiopeta; taxoids, paclitaxel (Taxol) and docetaxel (Taxotere); chlorambucil; gemcitabine (Gemzar); 6-thioguanine; mercaptopurine; methotrexate; platinum analogs such as cisplatin and carboplatin; platinum; ifosfamide; mitroxantrone; vancristine; vinorelbine (Navelbine); novantrone; teniposide; edatrexate; daunomycin; aminopterin; xeoloda; ibandronate; CPT-11; topoisomerase inhibitor RFS 2000; difluoromethylomithine (DMFO); retinoids such as retinoic acid; capecitabine and FOLFIRI (fluorouracil, leucovorin, and irinotecan);
(2) Anti-hormonal agents selected from the group consisting of: anti-estrogens and selective estrogen receptor modulators (SERMs), including, for example, tamoxifen (including Nolvadex), raloxifene, droloxifene, 4-hydroxytamoxifen, trioxifene, keoxifene, LY117018, onapristone, and toremifene; inhibitors of the enzymearomatase, which regulates estrogen production in the adrenal glands, such as, for example, 4(5)-imidazoles, aminoglutethimide, megestrol acetate, exemestane, formestane, fadrozole, vorozole, letrozole and anastrozole, and anti-androgens such as flutamide, nilutamide, bicalutamide, leuprolide, and goserelin;
(3) Anti-angiogenic agents selected from the group consisting of: retinoid acid and derivatives thereof, 2-methoxyestradiol, ANGIOSTATIN, ENDOSTATIN, suramin, squalamine, tissue inhibitors of metalloproteinase-1, tissue inhibitors of metalloproteinase-2, plasminogen activator inhibitor-1, plasminogen activator inhibitor-2, cartilage-derived inhibitors, paclitaxel (nab-paclitaxel), platelet factor 4, protamine sulphate (clupeine), sulphated chitin derivatives (prepared from queen crab shells), sulphated polysaccharide peptidoglycan complex (sp-pg), staurosporine, modulators of matrix metabolism, including for example, proline analogs ((1-azetidine-2-carboxylic acid (LACA), cishydroxyproline, d,I-3,4-dehydroproline, thiaproline, .alpha.-dipyridyl, beta-aminopropionitrile fumarate, 4-propyl-5-(4-pyridinyl)-2(3h)-oxazolone; methotrexate, mitoxantrone, heparin, interferons, 2 macroglobulin-serum, chimp-3, chymostatin, beta-cyclodextrin tetradecasulfate, eponemycin; fumagillin, gold sodium thiomalate, d-penicillamine (CDPT), beta-1-anticollagenase-serum, alpha-2-antiplasmin, bisantrene, lobenzarit disodium, n-2-carboxyphenyl-4-chloroanthronilic acid disodium or “CCA”, thalidomide; angiostatic steroid, carboxynaminolmidazole; metalloproteinase inhibitors such as BB94, antibodies, preferably monoclonal antibodies against these angiogenic growth factors: beta-FGF, alpha-FGF, FGF-5, VEGF isoforms, VEGF-C, HGF/SF, Ang-1/Ang-2 and the compounds disclosed in Ferrara N. and Alitalo, K. “Clinical application of angiogenic growth factors and their inhibitors” (1999) Nature Medicine 5:1359-1364;
(4) Anti-fibrotic agents selected from the group consisting of: beta-aminopropionitrile (BAPN), primary amines reacting with the carbonyl group of the active site of the lysyl oxidases, and more particularly those which produce, after binding with the carbonyl, a product stabilized by resonance, such as the following primary amines: emylenemamine, hydrazine, phenylhydrazine, and their derivatives, semicarbazide, and urea derivatives, aminonitriles, such as beta-aminopropionitrile (BAPN), or 2-nitroethylamine, unsaturated or saturated haloamines, such as 2-bromo-ethylamine, 2-chloroethylamine, 2-trifluoroethylamine, 3-bromopropylamine, p-halobenzylamines, selenohomocysteine lactone, copper chelating agents, indirect inhibitors such as compounds blocking the aldehyde derivatives originating from the oxidative deamination of the lysyl and hydroxylysyl residues by the lysyl oxidases, such as the thiolamines, in particular D-penicillamine, or its analogues such as 2-amino-5-mercapto-5-methylhexanoic acid, D-2-amino-3-methyl-3-((2-acetamidoethyl)dithio)butanoic acid, p-2-amino-3-methyl-3-((2-aminoethyl)dithio)butanoic acid, sodium-4-((p-1-dimethyl-2-amino-2-carboxyethyl)dithio)butane sulphurate, 2-acetamidoethyl-2-acetamidoethanethiol sulphanate, sodium-4-mercaptobutanesulphinate trihydrate, the compounds disclosed in U.S. Pat. Nos. 4,965,288, 4,997,854, 4,943,593, 5,021,456; 5,5059,714; 5,120,764; 5,182,297; 5,252,608 and U.S. Patent Application No. 2004/0248871;
(5) Therapeutic antibodies selected from the group consisting of: abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, clivatuzumab, conatumumab, daratumumab, drozitumab, duligotumab, dusigitumab, detumomab, dacetuzumab, dalotuzumab, ecromeximab, elotuzumab, ensituximab, ertumaxomab, etaracizumab, farietuzumab, ficlatuzumab, figitumumab, flanvotumab, futuximab, ganitumab, gemtuzumab, girentuximab, glembatumumab, ibritumomab, igovomab, imgatuzumab, indatuximab, inotuzumab, intetumumab, ipilimumab, iratumumab, labetuzumab, lexatumumab, lintuzumab, lorvotuzumab, lucatumumab, mapatumumab, matuzumab, milatuzumab, minretumomab, mitumomab, moxetumomab, narnatumab, naptumomab, necitumumab, nimotuzumab, nofetumomab, ocaratuzumab, ofatumumab, olaratumab, onartuzumab, oportuzumab, oregovomab, panitumumab, parsatuzumab, patritumab, pemtumomab, pertuzumab, pintumomab, pritumumab, racotumomab, radretumab, rilotumumab, rituximab, robatumumab, satumomab, sibrotuzumab, siltuximab, simtuzumab, solitomab, tacatuzumab, taplitumomab, tenatumomab, teprotumumab, tigatuzumab, tositumomab, trastuzumab, tucotuzumab, ublituximab, veltuzumab, vorsetuzumab, votumumab, zalutumumab, veltuzumab, apolizumab, epratuzumab, tositumomab, galiximab, lumiliximab, milatuzumab, obinutuzumab, ofatumumab, CC49 and 3F8, wherein the antibody may be further labeled or combined with a radioisotope particle, such as indium In 111, yttrium Y 90, iodine I-131;
(6); JAK inhibitors selected from the group consisting of: ruxolitinib, fedratinib, tofacitinib, baricitinib, lestaurtinib, pacritinib, momelotinib, XL019, AZD1480, INCB039110, LY2784544, BMS911543, and NS018;
(7) Hedgehog inhibitors selected from the group consisting of: saridegib;
(8) Histone deacetylase (HDAC) inhibitors selected from the group consisting of: pracinostat, romidepsin, vorinostat and panobinostat;
(9) Tyrosine kinase inhibitors selected from the group consisting of: lestaurtinib, gefitinib, erlotinib and sunitinib;
(10) Discoidin domain receptor (DDR) inhibitors selected from the group consisting of: the inhibitors disclosed in US2009/0142345, US2011/0287011, WO2013/027802, WO2013/034933, and U.S. Provisional Application No. 61/705,044;
(11) MMP9 inhibitors selected from the group consisting of: marimastat (BB-2516), cipemastat (Ro 32-3555), and the inhibitors described in WO2012/027721;
(12) LOXL inhibitors selected from the group consisting of: the antibodies described in WO2009/017833, the antibodies described in WO2009/017833, WO2009/035791 and WO/2011/097513;
(13) ASK1 inhibitors selected from the group consisting of: the compounds described in WO2011/008709 and WO/2013/112741;
(14) PI3K inhibitors selected from the group consisting of: the compounds described in U.S. Pat. No. 7,932,260, U.S. Provisional Application Nos. 61/543,176; 61/581,528; 61/745,429; 61/745,437; and 61/835,333, PI3K II, TGR-1202, AMG-319, GSK2269557, X-339, X-414, RP5090, KAR4141, XL499, OXY111A, duvelisib, IPI-443, GSK2636771, BAY 10824391, TGX221, RG-7666, CUDC-907, PQR-309, DS-7423, panulisib, AZD-8186, CLR-457, pictilisib, neratinib, rigosertib, rigosertib sodium, EN-3342, UCB-5857, taselisib, INCB-040093, pilaralisib, BAY-1082439, puquitinib mesylate, XL-765, gedatolisib, VS-5584, copanlisib, CAI orotate, alpelisib, buparlisib, BAY 80-6946, BYL719, PX-866, RG7604, MLN1117, WX-037, AEZS-129, PA799, ZSTK474, RP-6530, AS252424, LY294002, TG100115, LY294002, BEZ235, XL147 (SAR245408), SAR-245409, GDC-0941, BKM120, CH5132799, XL756, MLN-1117, SF-1126, RV-1729, sonolisib, GDC-0980, CLR-1401, perifosine and wortmannin;
(15) BTK inhibitors selected from the group consisting of: ibrutinib, HM71224, ONO-4059 and CC-292;
(16) SYK inhibitors selected from the group consisting of: tamatinib (R406), fostamatinib (R788), PRT062607, BAY-61-3606, NVP-QAB 205 AA, R112, R343, and the compounds described in U.S. Pat. No. 8,450,321;
(17) mTOR inhibitors selected from the group consisting of: temsirolimus, everolimus, ridaforolimus, deforolimus, OSI-027, AZD2014, CC-223, RAD001, LY294002, BEZ235, rapamycin, Ku-0063794, and PP242;
(18) AKT inhibitors selected from the group consisting of: perifosine, MK-2206, GDC-0068 and GSK795;
(19) MEK inhibitors selected from the group consisting of: trametinib, selumetinib, cobimetinib, MEK162, PD-325901, PD-035901, AZD6244, and CI-1040;
(20) CDK inhibitors selected from the group consisting of: AT-7519, alvocidib, palbociclib and SNS-032;
(21) JNK inhibitors selected from the group consisting of: CC-401;
(22) MAPK inhibitors selected from the group consisting of: VX-702, SB203580 and SB202190;
(23) Raf inhibitors selected from the group consisting of: PLX4720;
(24) ROCK inhibitors selected from the group consisting of: Rho-15;
(25) Tie2 inhibitors selected from the group consisting of: AMG-Tie2-1;
(26) Myo-inositol signaling inhibitors such as phospholipase C blockers and Myoinositol analogues described in Powis, G., and Kozikowski A., (1994) New Molecular Targets for Cancer Chemotherapy ed., Paul Workman and David Kerr, CRC press 1994, London;
(27) Bcl-2 family protein inhibitors selected from the group consisting of: ABT-263, ABT-199 and ABT-737;
(28) IKK inhibitors selected from the group consisting of: BMS-345541;
(29) Proteasome inhibitors selected from the group consisting of: bortezomib;
(30) Protein kinase C (PKC) inhibitors selected from the group consisting of: bryostatin 1 and enzastaurin;
(31) Heat shock protein HSP90 inhibitors selected from the group consisting of: Geldanamycin;
(32) Combination drugs selected from the group consisting of: FR (fludarabine, rituximab), FCR (fludarabine, cyclophosphamide, rituximab), R-CHOP (rituximab plus CHOP), R-CVP (rituximab plus CVP), R-FCM (rituximab plus FCM), R-ICE (rituximab-ICE), CHOP (cyclophosphamide, doxorubicin, vincristine, prednisone), CVP (cyclophosphamide, vincristine and prednisone), FCM (fludarabine, cyclophosphamide, mitoxantrone), hyperCVAD (hyperfractionated cyclophosphamide, vincristine, doxorubicin, dexamethasone, methotrexate, cytarabine), ICE (iphosphamide, carboplatin and etoposide), MCP (mitoxantrone, chlorambucil, and prednisolone), and R MCP (R MCP); and
(33) other drugs for treating cancer selected from the group consisting of aldesleukin, alvocidib, CHIR-12.12, ha20, tiuxetan, PRO131921, SGN-40, WT-1 analog peptide vaccine, WT1126-134 peptide vaccine, autologous human tumor-derived HSPPC-96, GTOP-99 (MyVax®), antineoplaston AS2-1, antineoplaston A10, anti-thymocyte globulin, beta alethine, arsenic trioxide, amifostine, aminocamptothecin, lenalidomide, caspofungin, clofarabine, ixabepilone, cladribine, chlorambucil, Curcumin, vinorelbine, tipifamib, tanespimycin, sildenafil citrate, denileukin diftitox, simvastatin, epoetin alfa, fenretinide, filgrastim, mesna, mitoxantrone, lenalidomide, fludarabine, mycophenolate mofetil, nelarabine, octreotide, oxaliplatin, pegfilgrastim, recombinant interleukin-12, recombinant interleukin-11, recombinant flt3 ligand, recombinant human thrombopoietin, sargramostim, lymphokine-activated killer cells, omega-3 fatty acids, recombinant interferon alfa, therapeutic allogeneic lymphocytes and cyclosporine analogs.

In a particular embodiment, a compound of the present disclosure, or a pharmaceutically acceptable salt thereof, is combined with one, two, three, four or more additional therapeutic agents selected from ibrutinib, aldesleukin, alvocidib, antineoplaston AS2-1, antineoplaston A10, anti-thymocyte globulin, amifostine trihydrate, aminocamptothecin, arsenic trioxide, beta alethine, ABT-263, ABT-199, ABT-737, BMS-345541, bortezomib, bryostatin 1, busulfan, carboplatin, campath-1H, CC-5103, carmustine, caspofungin acetate, clofarabine, cisplatin, Cladribine (Leustarin), Chlorambucil (Leukeran), Curcumin, cyclosporine, Cyclophosphamide (Cyloxan, Endoxan, Endoxana, Cyclostin), denileukin diftitox, dexamethasone, DT PACE, docetaxel, dolastatin 10, Doxorubicin (Adriamycin®, Adriblastine), doxorubicin hydrochloride, enzastaurin, epoetin alfa, etoposide, everolimus (RAD001), fenretinide, filgrastim, melphalan, mesna, flavopiridol, fludarabine (Fludara), Geldanamycin (17 AAG), ifosfamide, irinotecan hydrochloride, ixabepilone, lenalidomide (Revlimid®), lymphokine-activated killer cells, melphalan, methotrexate, mitoxantrone hydrochloride, motexafin gadolinium, mycophenolate mofetil, nelarabine, oblimersen Obatoclax, oblimersen, octreotide acetate, omega-3 fatty acids, oxaliplatin, paclitaxel, PD0332991, PEGylated liposomal doxorubicin hydrochloride, pegfilgrastim, Pentstatin (Nipent), perifosine, Prednisolone, Prednisone, selicilib, recombinant interferon alfa, recombinant interleukin-12, recombinant interleukin-11, recombinant flt3 ligand, recombinant human thrombopoietin, rituximab, sargramostim, sildenafil citrate, simvastatin, sirolimus, Styryl sulphones, tacrolimus, tanespimycin, temsirolimus, thalidomide, therapeutic allogeneic lymphocytes, thiotepa, tipifamib, Vincristine, vincristine sulfate, vinorelbine ditartrate, Vorinostat (SAHA), vorinostat, FR (fludarabine, rituximab), CHOP (cyclophosphamide, doxorubicin, vincristine, prednisone), CVP (cyclophosphamide, vincristine and prednisone), FCM (fludarabine, cyclophosphamide, mitoxantrone), FCR (fludarabine, cyclophosphamide, rituximab), hyperCVAD (hyperfractionated cyclophosphamide, vincristine, doxorubicin, dexamethasone, methotrexate, cytarabine), ICE (iphosphamide, carboplatin and etoposide), MCP (mitoxantrone, chlorambucil, and prednisolone), R-CHOP (rituximab plus CHOP), R-CVP (rituximab plus CVP), R-FCM (rituximab plus FCM), R-ICE (rituximab-ICE), and R MCP (R MCP).

Any of the methods of treatment provided may be used to treat cancer at various stages. By way of example, the cancer stage includes but is not limited to early, advanced, locally advanced, remission, refractory, reoccurred after remission and progressive.

In addition, the subject may be a human who is undergoing one or more standard therapies, such as chemotherapy, radiotherapy, immunotherapy, surgery, or combination thereof. Accordingly, one or more anti-cancer agents may be administered before, during, or after administration of chemotherapy, radiotherapy, immunotherapy, surgery or combination thereof.

The therapeutic treatments can be supplemented or combined with any of the abovementioned therapies with stem cell transplantation or treatment. One example of modified approach is radioimmunotherapy, wherein a monoclonal antibody is combined with a radioisotope particle, such as indium In 111, yttrium Y 90, iodine I-131. Examples of combination therapies include, but are not limited to, Iodine-131 tositumomab (Bexxar®), Yttrium-90 ibritumomab tiuxetan (Zevalin®), Bexxar® with CHOP.

Other therapeutic procedures include peripheral blood stem cell transplantation, autologous hematopoietic stem cell transplantation, autologous bone marrow transplantation, antibody therapy, biological therapy, enzyme inhibitor therapy, total body irradiation, infusion of stem cells, bone marrow ablation with stem cell support, in vitro-treated peripheral blood stem cell transplantation, umbilical cord blood transplantation, immunoenzyme technique, pharmacological study, low-LET cobalt-60 gamma ray therapy, bleomycin, conventional surgery, radiation therapy, and nonmyeloablative allogeneic hematopoietic stem cell transplantation.

Also provided herein is a compound of the present disclosure (e.g., a compound of Formula (I)), or a pharmaceutically acceptable salt thereof, and one or more additional therapeutic agents for treating cancer, for use in a method of treating cancer.

Also provided herein is a compound of the present disclosure (e.g., a compound of Formula (I)), or a pharmaceutically acceptable salt thereof, for use in a method of treating cancer, wherein the compound or a pharmaceutically acceptable salt thereof is administered simultaneously, separately or sequentially with one or more additional therapeutic agents for treating cancer.

### XI. Kits

The present disclosure provides a kit comprising a compound of the present disclosure or a pharmaceutically acceptable salt thereof. The kit may further comprise instructions for use, e.g., for use in modulating a toll-like receptor (e.g. TLR-8), such as for use in treating a disease, disorder, or condition. In certain embodiments the use is for treating a HIV, HBV, or HCV infection. In certain embodiments the use is for treating a HBV infection. The instructions for use are generally written instructions, although electronic storage media (e.g., magnetic diskette or optical disk) containing instructions are also acceptable.

The present disclosure also provides a pharmaceutical kit comprising one or more containers comprising a compound of the present disclosure or a pharmaceutically acceptable salt thereof. Optionally associated with such container(s) can be a notice in the form prescribed by a governmental agency regulating the manufacture, use or sale of pharmaceuticals, which notice reflects approval by the agency for the manufacture, use or sale for human administration. Each component (if there is more than one component) can be packaged in separate containers or some components can be combined in one container where cross-reactivity and shelf life permit. The kits may be in unit dosage forms, bulk packages (e.g., multi-dose packages) or sub-unit doses. Kits may also include multiple unit doses of the compounds and instructions for use and be packaged in quantities sufficient for storage and use in pharmacies (e.g., hospital pharmacies and compounding pharmacies).

### XII. Compound Preparation

Also provided are articles of manufacture comprising a unit dosage of a compound of the present disclosure or a pharmaceutically acceptable salt thereof, in suitable packaging for use in the methods described herein. Suitable packaging is known in the art and includes, for example, vials, vessels, ampules, bottles, jars, flexible packaging and the like. An article of manufacture may further be sterilized and/or sealed.

The embodiments are also directed to processes and intermediates useful for preparing the subject compounds or pharmaceutically acceptable salts thereof.

Many general references providing commonly known chemical synthetic schemes and conditions useful for synthesizing the disclosed compounds are available (see, e.g., Smith, March's Advanced Organic Chemistry: Reactions, Mechanisms, and Structure, 7^{th} edition, Wiley-Interscience, 2013.)

Compounds as described herein can be purified by any of the means known in the art, including chromatographic means, such as high performance liquid chromatography (HPLC), preparative thin layer chromatography, flash column chromatography and ion exchange chromatography. Any suitable stationary phase can be used, including normal and reversed phases as well as ionic resins. Most typically the disclosed compounds are purified via silica gel and/or alumina chromatography. See, e.g., Introduction to Modern Liquid Chromatography, 2nd ed., ed. L. R. Snyder and J. J. Kirkland, John Wiley and Sons, 1979; and Thin Layer Chromatography, E. Stahl (ed.), Springer-Verlag, New York, 1969.

During any of the processes for preparation of the subject compounds, it may be necessary and/or desirable to protect sensitive or reactive groups on any of the molecules concerned. This may be achieved by means of conventional protecting groups as described in standard works, such as T. W. Greene and P. G. M. Wuts, “Protective Groups in Organic Synthesis,” 4^{th} ed., Wiley, New York 2006. The protecting groups may be removed at a convenient subsequent stage using methods known from the art.

Exemplary chemical entities useful in methods of the embodiments will now be described by reference to illustrative synthetic schemes for their general preparation herein and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Furthermore, one of skill in the art will recognize that the transformations shown in the schemes below may be performed in any order that is compatible with the functionality of the particular pendant groups. Each of the reactions depicted in the general schemes is preferably run at a temperature from about 0° C. to the reflux temperature of the organic solvent used. Unless otherwise specified, the variables are as defined above in reference to Formulas (I) or (J).

Representative syntheses of compounds of the present disclosure are described in schemes below, and the particular examples that follow.

Scheme 1 shows a representative synthesis of the compounds of the embodiments. The methodology is compatible with a wide variety of functionalities.

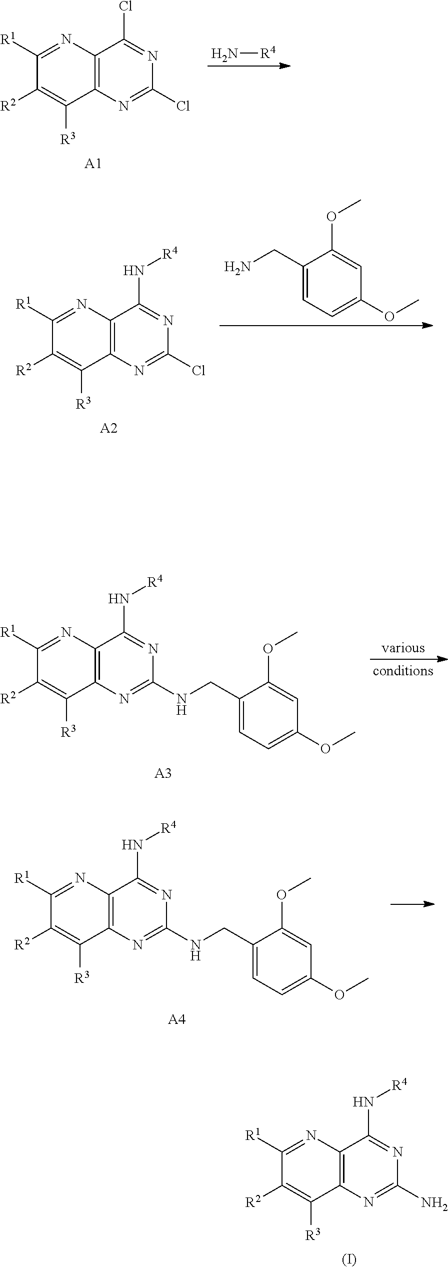

In Scheme 1, compounds of formula A1 (where R¹, R², and R³ are as defined herein or are suitably protected derivatives of R¹, R², and R³) are converted to the corresponding 4-amino, 2-chloro heterocycle by reaction with a nucleophilic amine in the presence of a suitable base (such as DIPEA) at room temperature. The compound of formula A2 is then treated with 2,4-dimethoxybenzylamine at elevated temperature resulting in a 2,4-diaminopyrimidine of formula A3. In cases where R¹, R², and R³ is a diversifiable chemical group such as Cl or Br, further replacement of R¹, R², and R³ by a variety of methods including cyanation, nucleophilic aromatic displacement, and metal catalyzed cross coupling reactions such as Suzuki couplings is carried out to provide products of formula A4. Treatment with a suitable acid (such as trifluoroacetic acid) leads to certain compounds of Formula (I) or (J). Where suitable, other leaving groups may be used in place of the Cl group(s) of A1.

Scheme 2 describes a general route which is used to prepare certain compounds of Formula (I) or (J).

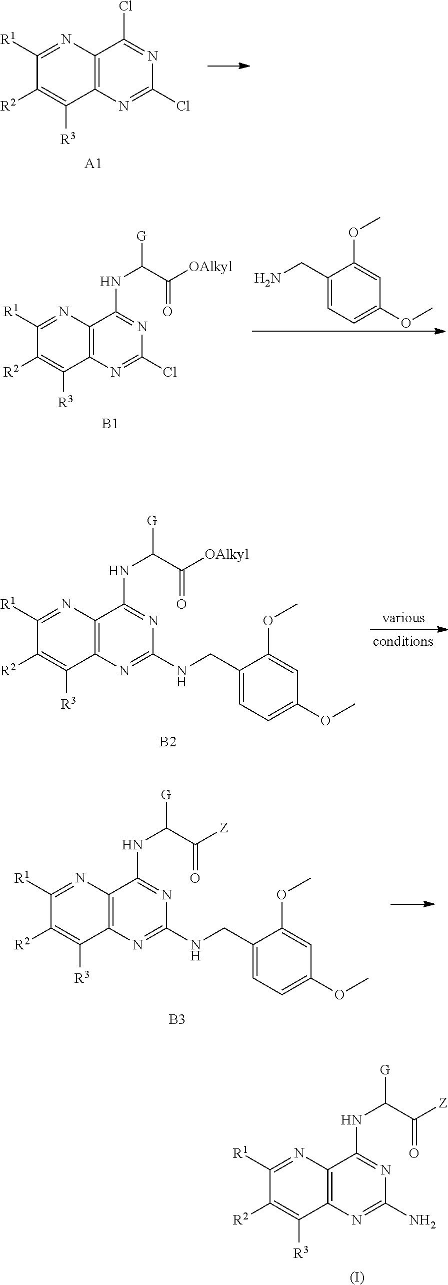

2,4-dichloro pyrido-pyrimidines of formula A1 (where R¹, R², and R³ are as defined herein or are suitably protected derivatives of R¹, R², and R³) are converted to the corresponding 4-amino, 2-chloro heterocycle by reaction with an amino acid ester (such as L-norvaline methyl ester) in the presence of a suitable base (such as DIPEA) at room temperature to provide a compound of formula B1, where G is an the sidechain of the amino acid. The compound of formula B1 is then treated with 2,4-dimethoxybenzylamine in a microwave reactor at a suitable temperature (such as about 135° C.), resulting in a 2,4-diaminopyrimidine of formula B2. Hydrolysis of the ester group via treatment with a suitable base (such as aqueous KOH/THF) provides product of formula B3 where Z is hydroxyl. Further reaction of the resulting carboxylic acid leads to modification of Z via HATU-promoted amide formation with various amines. Protecting group removal with a suitable acid (such as trifluoroacetic acid) at room temperature then leads to certain compounds of Formula (J) or (I).

Scheme 3 shows a representative synthesis of the compounds of the embodiments. The methodology is compatible with a wide variety of functionalities.

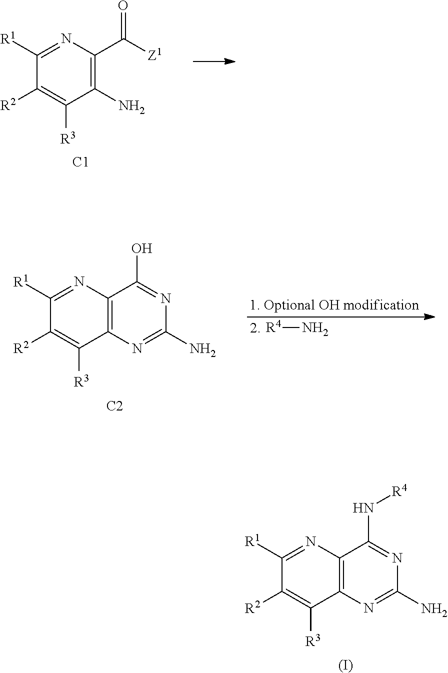

An amide of formula C1 (where R¹, R², and R³ are as defined herein or are suitably protected derivatives of R¹, R², and R³, and Z¹ is NH² or O-alkyl) is converted to a compound of formula C2, under suitable reaction conditions. For example, the compound of formula C1 is contacted with chloroformamidine hydrochloride under suitable conditions to provide C2. The hydroxyl group may be further modified, for example by introducing any suitable leaving group, such as a tosyl group, prior to contacting with R⁴—NH₂. Alternatively, R⁴—NH₂ may be directly coupled to C2 im the presence of a suitable coupling agent, for example, BOP reagent, under suitable conditions.

Additionally, a compound of Formula A1 (where R¹, R², and R³ are as defined herein or are suitably protected derivatives of R¹, R², and R³) may be prepared as described in the scheme below. It is understood that A1 may be further modified to prepare compounds of Formula (I) as more fully described herein.

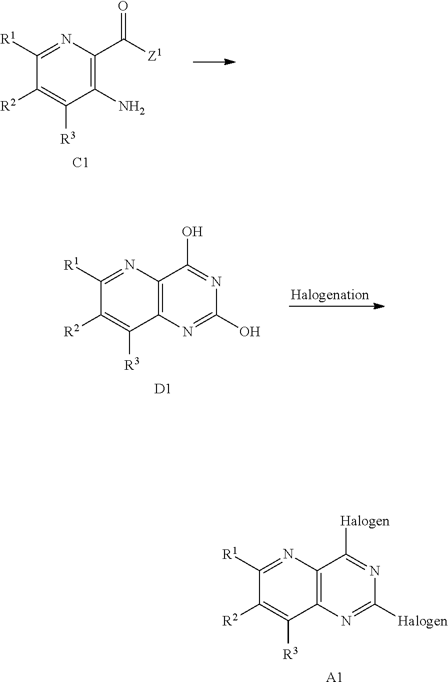

As described above C1 is contacted with a suitable agent, such as triphosgene and dioxane, to result in a compound of D1. The compound D1 may be further halogenated under suitable conditions, such as treatment with POCl₃ and PCl₅, to provide a compound of formula A1.

In certain instances, the above processes further involve the step of forming a salt of a compound of the present disclosure. Embodiments are directed to the other processes described herein; and to the product prepared by any of the processes described herein.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, 5^{th} edition, New York: Oxford University Press, 2009; Smith, March's Advanced Organic Chemistry: Reactions, Mechanisms, and Structure, 7^{th} edition, Wiley-Interscience, 2013.

The Examples provided herein describe the synthesis of compounds disclosed herein as well as intermediates used to prepare the compounds. It is to be understood that individual steps described herein may be combined. It is also to be understood that separate batches of a compound may be combined and then carried forth in the next synthetic step.

In the following description of the Examples, specific embodiments are described. These embodiments are described in sufficient detail to enable those skilled in the art to practice certain embodiments of the present disclosure. Other embodiments may be utilized and logical and other changes may be made without departing from the scope of the disclosure. The following description is, therefore, not intended to limit the scope of the present disclosure.

diastereomer as the desired product, although the stereochemistry of the enantiomer or diastereomer was not determined in all cases. When the stereochemistry of the specific stereocenter in the enantiomer or diastereomer is not determined, the compound is drawn without showing any stereochemistry at that specific stereocenter even though the compound can be substantially enantiomerically or diastereomerically pure.

### Example 1

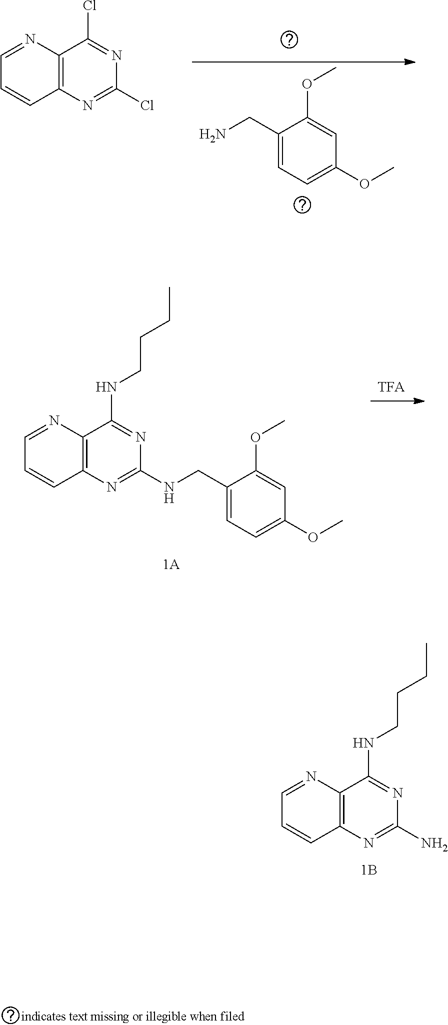 [SMILES: Clc1nc(Cl)c2ncccc2n1 ; COc1ccc(CN)c(OC)c1 ; CCCCNc1nc(NCc2ccc(OC)cc2OC)nc2cccnc12 ; CCCCNc1nc(N)nc2cccnc12]

Synthesis of N⁴-butyl-N²-(2,4-dimethoxybenzyl)pyrido[3,2-d]pyrimidine-2,4-diamine (1A): To a solution of 2,4-dichloropyrido[3,2-d]pyrimidine (CAS #39551-54-7, supplied by Astatech, Inc.) (50 mg, 0.25 mmol) in THF (2 mL) was added butan-1-amine (0.03 mL, 0.28 mmol) and N,N-diisopropylethylamine (0.13 ml, 0.75 mmol). After stirring at room temperature for 30 minutes, 2,4-dimethoxybenzylamine (0.19 ml, 1.25 mmol) and N,N-diisopropylethylamine (0.13 ml, 0.75 mmol) were added and the mixture was heated to 100° C. After 16 hours, the reaction was cooled to room temperature, diluted with ethyl acetate, washed with water and brine, dried over Na₂SO₄, and concentrated in vacuo. The product (1A) was obtained after flash chromatography. MS (m/z): 368.14 [M+H]⁺.

Synthesis of N⁴-butylpyrido[3,2-d]pyrimidine-2,4-diamine (1B): 1A was dissolved in trifluoroacetic acid (3 mL). After 30 minutes, the reaction was diluted with water and methanol. After 60 minutes, the mixture was concentrated in vacuo. The residue was then co-evaporated with methanol three times and filtered in methanol to afford the title product 1B as a trifluoroacetic acid salt. ¹H NMR (400 MHz, Methanol-d₄) δ 8.59 (dd, J=4.4, 1.4 Hz, 1H), 7.82 (dd, J=8.5, 1.4 Hz, 1H), 7.72 (dd, J=8.5, 4.4 Hz, 1H), 3.66 (t, J=7.3 Hz, 2H), 1.78-1.62 (m, 2H), 1.43 (dq, J=14.7, 7.4 Hz, 2H), 0.98 (t, J=7.4 Hz, 3H). MS (m/z): 218.10 [M+H]⁺. ¹⁹F NMR (377 MHz, Methanol-d4) δ −77.6.

### Example 2

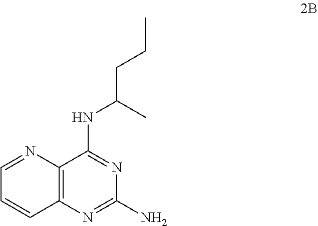 [SMILES: CCCC(C)Nc1nc(N)nc2cccnc12]

Synthesis of N²-(2,4-dimethoxybenzyl)-N⁴-(pentan-2-yl)pyrido[3,2-d]pyrimidine-2,4-diamine (2A): 2A was synthesized following the procedure described above for preparation of 1A, replacing butan-1-amine with 2-aminopentane. MS (m/z) 382.17 [M+H]⁺.

Synthesis of N⁴-(pentan-2-yl)pyrido[3,2-d]pyrimidine-2,4-diamine (2B): 2B was prepared following the procedure described for 1B to yield the title compound (2B) as its TFA salt. ¹H NMR (400 MHz, Methanol-d4) δ 8.61 (dd, J=4.4, 1.4 Hz, 1H), 7.84 (dd, J=8.5, 1.4 Hz, 1H), 7.74 (dd, J=8.5, 4.4 Hz, 1H), 4.60-4.46 (m, 1H), 1.74 (dtd, J=13.5, 8.3, 6.7 Hz, 1H), 1.68-1.55 (m, 1H), 1.44 (d, J=7.4 Hz, 2H), 1.32 (d, J=6.6 Hz, 3H), 0.95 (t, J=7.4 Hz, 3H). MS (m/z) 232.11 [M+H]⁺. ¹⁹F NMR (377 MHz, Methanol-d4) δ −77.5.

### Example 3

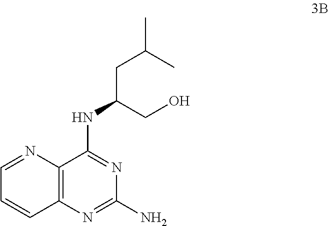 [SMILES: CC(C)C[C@@H](CO)Nc1nc(N)nc2cccnc12]

Synthesis of (S)-2-((2-((2,4-dimethoxybenzyl)amino)pyrido[3,2-d]pyrimidin-4-yl)amino)-4-methylpentan-1-ol (3A): 3A was synthesized following the above procedure for 1A, replacing butan-1-amine with (S)-(+)-leucinol. MS (m/z) 412.19 [M+H]⁺.

Synthesis of (S)-2-((2-aminopyrido[3,2-d]pyrimidin-4-yl)amino)-4-methylpentan-1-ol (3B): 3B was synthesized using the procedure described above for the preparation of 1B to yield the title compound (3B) as its TFA salt. ¹H NMR (400 MHz, Methanol-d4) δ 8.62 (dd, J=4.4, 1.3 Hz, 1H), 7.84 (dd, J=8.5, 1.4 Hz, 1H), 7.74 (dd, J=8.5, 4.4 Hz, 1H), 4.74-4.58 (m, 1H), 3.71 (h, J=6.2 Hz, 2H), 1.76-1.58 (m, 2H), 1.52 (tq, J=10.6, 3.5 Hz, 1H), 0.98 (t, J=6.4 Hz, 6H). MS (m/z) 262.15 [M+H]⁺. ¹⁹F NMR (377 MHz, Methanol-d4) δ −77.6

### Example 4

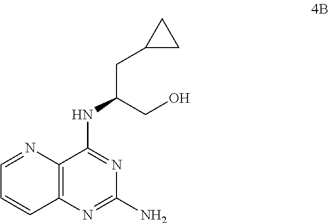 [SMILES: Nc1nc(N[C@H](CO)CC2CC2)c2ncccc2n1]

Synthesis of (S)-3-cyclopropyl-2-((2-((2,4-dimethoxybenzyl)amino)pyrido[3,2-d]pyrimidin-4-yl)amino)propan-1-ol (4A): 4A was prepared using the procedure described above for the preparation of 1A, replacing butan-1-amine with (2S)-2-amino-3-cyclopropylpropan-1-ol HCl salt. MS (m/z) 410.20 [M+H]⁺

Synthesis of (S)-2-((2-aminopyrido[3,2-d]pyrimidin-4-yl)amino)-3-cyclopropylpropan-1-ol (4B): 4B was synthesized following the procedure described above for 1B to yield the title compound (4B) as its TFA salt. ¹H NMR (400 MHz, Methanol-d4) δ 8.62 (dd, J=4.4, 1.3 Hz, 1H), 7.85 (dd, J=8.5, 1.4 Hz, 1H), 7.75 (dd, J=8.5, 4.4 Hz, 1H), 4.63 (dq, J=7.3, 5.5 Hz, 1H), 3.81 (d, J=5.2 Hz, 2H), 1.65 (h, J=7.1 Hz, 2H), 0.78 (dddd, J=15.0, 10.1, 5.1, 2.1 Hz, 1H), 0.45 (dddd, J=11.1, 9.4, 7.9, 4.6 Hz, 2H), 0.19-0.07 (m, 2H). MS (m/z) 260.15 [M+H]⁺. ¹⁹F NMR (377 MHz, Methanol-d4) δ −77.6

### Example 5

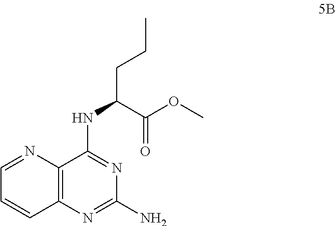 [SMILES: CCC[C@H](Nc1nc(N)nc2cccnc12)C(=O)OC]

Synthesis of (S)-methyl 2-((2-((2,4-dimethoxybenzyl)amino)pyrido[3,2-d]pyrimidin-4-yl)amino)pentanoate (5A): 5A was prepared following the general procedure described above for 1A, replacing butan-1-amine with (S)-methyl 2-aminopentanoate. MS (m/z) 426.19 [M+H]⁺.

Synthesis of (S)-methyl 2-((2-aminopyrido[3,2-d]pyrimidin-4-yl)amino)pentanoate (5B): 5B was prepared following the procedure described above for 1B to yield the title compound (5B) as its TFA salt. ¹H NMR (400 MHz, Methanol-d4) δ 8.66 (dd, J=4.4, 1.4 Hz, 1H), 7.88 (dd, J=8.5, 1.4 Hz, 1H), 7.79 (dd, J=8.5, 4.4 Hz, 1H), 5.02 (dd, J=8.7, 5.3 Hz, 1H), 3.78 (s, 3H), 2.13-1.92 (m, 2H), 1.56-1.39 (m, 2H), 0.99 (t, J=7.4 Hz, 3H). MS (m/z) 276.13 [M+H]⁺. ¹⁹F NMR (377 MHz, Methanol-d4) δ −77.8.

### Example 6

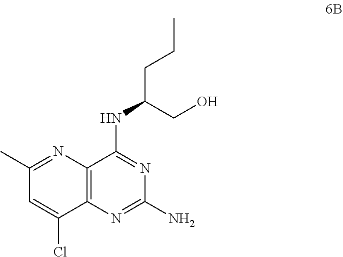 [SMILES: CCC[C@@H](CO)Nc1nc(N)nc2c(Cl)cc(C)nc12]

Synthesis of (S)-2-((8-chloro-2-((2,4-dimethoxybenzyl)amino)-6-methylpyrido[3,2-d]pyrimidin-4-yl)amino)pentan-1-ol (6A): 6A was prepared following the procedure described above for 1A, replacing butan-1-amine with (S)-methyl 2-aminopentanoate and instead starting from 2,4,8-trichloro-6-methylpyrido[3,2-d]pyrimidine in place of 2,4-dichloropyrido[3,2-d]pyrimidine. MS (m/z) 446.20 [M+H]⁺.

Synthesis of (S)-2-((2-amino-8-chloro-6-methylpyrido[3,2-d]pyrimidin-4-yl)amino)pentan-1-ol (6B): 6B was prepared following the procedure described above for 1B to yield the title compound (6B) as its TFA salt. ¹H NMR (400 MHz, Methanol-d₄) δ 7.84 (s, 1H), 4.55 (ddd, J=12.6, 7.2, 5.2 Hz, 1H), 3.75 (d, J=5.3 Hz, 3H), 1.79-1.67 (m, 3H), 1.51-1.35 (m, 3H), 0.98 (t, J=7.4 Hz, 4H). MS (m/z) 296.18 [M+H]⁺. ¹⁹F NMR (377 MHz, Methanol-d4) δ −77.6.

### Example 7

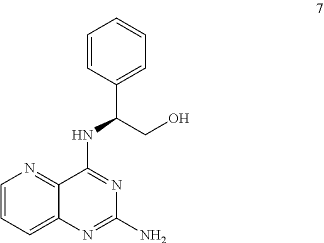 [SMILES: Nc1nc(N[C@H](CO)c2ccccc2)c2ncccc2n1]

Compound 7, (S)-2-((2-aminopyrido[3,2-d]pyrimidin-4-yl)amino)-2-phenylethanol, was prepared following the procedure for compound 1B reported above, instead replacing butan-1-amine with (S)-2-amino-2-phenylethanol to yield the title compound (7) as its TFA salt. ¹H NMR (400 MHz, Methanol-d4) δ 8.68 (dd, J=4.3, 1.5 Hz, 1H), 7.84 (dd, J=8.5, 1.5 Hz, 1H), 7.77 (dd, J=8.5, 4.4 Hz, 1H), 7.49-7.43 (m, 2H), 7.38-7.31 (m, 2H), 7.31-7.24 (m, 1H), 5.57 (dd, J=7.4, 4.8 Hz, 1H), 4.12-3.93 (m, 2H). ¹⁹F NMR (376 MHz, Methanol-d4) δ −77.7. MS (m/z) 282.1 [M+H]⁺.

### Example 8

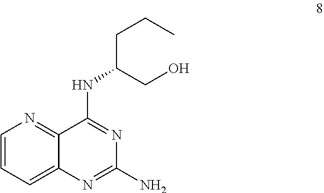 [SMILES: CCC[C@H](CO)Nc1nc(N)nc2cccnc12]

Compound 8, (R)-2-((2-aminopyrido[3,2-d]pyrimidin-4-yl)amino)pentan-1-ol, was prepared following the procedure for the synthesis of compound 1B reported above, instead replacing butan-1-amine with (R)-2-aminopentan-1-ol to yield the title compound (8) as its TFA salt. ¹H NMR (400 MHz, Methanol-d4) δ 8.64 (dd, J=4.4, 1.4 Hz, 1H), 7.83 (dd, J=8.5, 1.5 Hz, 1H), 7.76 (dd, J=8.5, 4.4 Hz, 1H), 4.55 (dq, J=7.4, 5.4 Hz, 1H), 3.78-3.69 (m, 2H), 1.77-1.65 (m, 2H), 1.52-1.36 (m, 2H), 0.98 (t, J=7.3 Hz, 3H). ¹⁹F NMR (376 MHz, Methanol-d4) δ −77.56. MS (m/z) 248.1 [M+H]⁺.

### Example 9

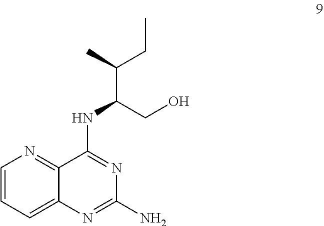 [SMILES: CC[C@H](C)[C@@H](CO)Nc1nc(N)nc2cccnc12]

Compound 9, (2S,3S)-2-((2-aminopyrido[3,2-d]pyrimidin-4-yl)amino)-3-methylpentan-1-ol, was prepared following the procedure for compound 1B reported above, instead replacing butan-1-amine with (2S,3S)-2-amino-3-methylpentan-1-ol to yield the title compound (9) as its TFA salt. ¹H NMR (400 MHz, Methanol-d4) δ 8.64 (dd, J=4.4, 1.4 Hz, 1H), 7.84 (dd, J=8.5, 1.4 Hz, 1H), 7.76 (dd, J=8.5, 4.4 Hz, 1H), 4.39 (dt, J=8.1, 5.0 Hz, 1H), 3.83 (d, J=5.0 Hz, 2H), 1.97-1.82 (m, 1H), 1.58 (dddd, J=16.8, 11.2, 7.6, 3.8 Hz, 1H), 1.33-1.16 (m, 2H), 1.03 (d, J=6.8 Hz, 3H), 0.94 (t, J=7.4 Hz, 3H). ¹⁹F NMR (376 MHz, Methanol-d4) δ −77.71. MS (m/z) 262.1 [M+H]⁺.

### Example 10

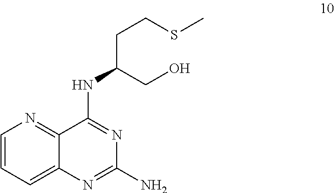 [SMILES: CSCC[C@@H](CO)Nc1nc(N)nc2cccnc12]

Compound 10, (S)-2-((2-aminopyrido[3,2-d]pyrimidin-4-yl)amino)-4-(methylthio)butan-1-ol, was prepared following the 2 step procedure for compound 1B reported above, replacing butan-1-amine with (S)-2-amino-4-(methylthio)butan-1-ol to yield the title compound (10) as its TFA salt. ¹H NMR (400 MHz, Methanol-d4) δ 8.64 (dd, J=4.4, 1.4 Hz, 1H), 7.83 (dd, J=8.5, 1.4 Hz, 1H), 7.76 (dd, J=8.5, 4.4 Hz, 1H), 4.66 (dq, J=8.1, 5.4 Hz, 1H), 3.76 (d, J=5.3 Hz, 2H), 2.65-2.52 (m, 2H), 2.11-1.98 (m, 5H). ¹⁹F NMR (376 MHz, Methanol-d4) δ −77.63. MS (m/z) 280.1 [M+H]⁺.

### Example 11

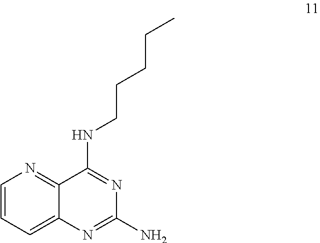 [SMILES: CCCCCNc1nc(N)nc2cccnc12]

Compound 11, N⁴-pentylpyrido[3,2-d]pyrimidine-2,4-diamine, was prepared following the procedure for compound 1B reported above, instead replacing butan-1-amine with n-pentylamine to yield the title compound (11) as its TFA salt. ¹H NMR (400 MHz, Methanol-d4) δ 8.62 (dd, J=4.4, 1.4 Hz, 1H), 7.81 (dd, J=8.5, 1.4 Hz, 1H), 7.74 (dd, J=8.5, 4.4 Hz, 1H), 3.67 (dd, J=7.8, 6.8 Hz, 2H), 1.80-1.66 (m, 2H), 1.49-1.32 (m, 4H), 0.99-0.85 (m, 3H). ¹⁹F NMR (376 MHz, Methanol-d4) δ −77.58. MS (m/z) 232.1 [M+H]⁺.

### Example 12

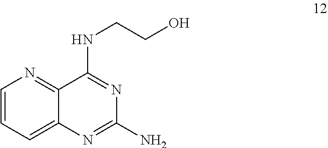 [SMILES: Nc1nc(NCCO)c2ncccc2n1]

Compound 12, 2-((2-aminopyrido[3,2-d]pyrimidin-4-yl)amino)ethanol, was prepared following the procedure for compound 1B reported above, instead replacing butan-1-amine with ethanolamine to yield the title compound (12) as its TFA salt. ¹H NMR (400 MHz, Methanol-d4) δ 8.64 (dd, J=4.3, 1.5 Hz, 1H), 7.88-7.72 (m, 2H), 3.82 (d, J=2.3 Hz, 4H). ¹⁹F NMR (376 MHz, Methanol-d4) δ −77.58. MS (m/z) 206.0 [M+H]⁺.

### Example 13

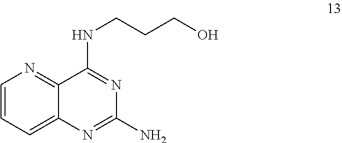 [SMILES: Nc1nc(NCCCO)c2ncccc2n1]

Compound 13, 3-((2-aminopyrido[3,2-d]pyrimidin-4-yl)amino)propan-1-ol, was prepared following the 2 step procedure for compound 1B reported above, instead replacing butan-1-amine with propanolamine to yield the title compound (13) as its TFA salt. ¹H NMR (400 MHz, Methanol-d4) δ 8.62 (td, J=4.6, 1.4 Hz, 1H), 7.87-7.70 (m, 2H), 3.80 (dt, J=11.7, 6.8 Hz, 2H), 3.70 (t, J=6.0 Hz, 2H), 2.00-1.88 (m, 2H). ¹⁹F NMR (376 MHz, Methanol-d4) δ −77.58. MS (m/z) 220.1 [M+H]⁺.

### Example 14

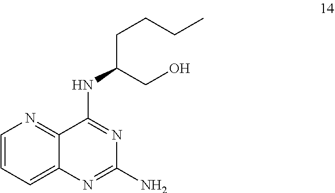 [SMILES: CCCC[C@@H](CO)Nc1nc(N)nc2cccnc12]

Compound 14, (S)-2-((2-aminopyrido[3,2-d]pyrimidin-4-yl)amino)hexan-1-ol, was prepared following the procedure for compound 1B reported above, instead replacing butan-1-amine with (S)-2-aminohexan-1-ol to yield the title compound (14) as its TFA salt. ¹H NMR (400 MHz, Methanol-d4) δ 8.63 (dd, J=4.4, 1.4 Hz, 1H), 7.84 (dd, J=8.5, 1.4 Hz, 1H), 7.76 (dd, J=8.5, 4.4 Hz, 1H), 4.53 (dq, J=8.6, 5.4 Hz, 1H), 3.79-3.68 (m, 2H), 1.87-1.61 (m, 2H), 1.52-1.31 (m, 4H), 1.01-0.85 (m, 3H). ¹⁹F NMR (376 MHz, Methanol-d4) δ −77.63. MS (m/z) 262.2 [M+H]⁺.

### Example 15

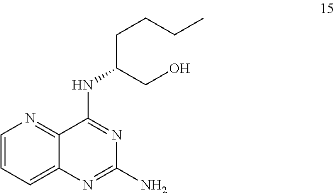 [SMILES: CCCC[C@H](CO)Nc1nc(N)nc2cccnc12]

Compound 15, (R)-2-((2-aminopyrido[3,2-d]pyrimidin-4-yl)amino)hexan-1-ol, was prepared following the procedure for compound 1B reported above, instead replacing butan-1-amine with (R)-2-aminohexan-1-ol to yield the title compound (15) as its TFA salt. ¹H NMR (400 MHz, Methanol-d4) δ 8.66-8.59 (m, 1H), 7.84 (dd, J=8.5, 1.4 Hz, 1H), 7.77 (td, J=8.8, 4.4 Hz, 1H), 4.59-4.42 (m, 1H), 3.81-3.68 (m, 2H), 1.90-1.65 (m, 2H), 1.49-1.35 (m, 4H), 1.03-0.82 (m, 3H). ¹⁹F NMR (376 MHz, Methanol-d4) δ −77.60. MS (m/z) 262.2 [M+H]⁺.

### Example 16

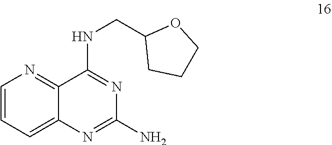 [SMILES: Nc1nc(NCC2CCCO2)c2ncccc2n1]

Compound 16, N⁴-((tetrahydrofuran-2-yl)methyl)pyrido[3,2-d]pyrimidine-2,4-diamine, was prepared following the procedure for compound 1B reported above, instead replacing butan-1-amine with (tetrahydrofuran-2-yl)-methanamine to yield the title compound (16) as its TFA salt. ¹H NMR (400 MHz, Methanol-d4) δ 8.62 (dd, J=4.4, 1.4 Hz, 1H), 7.83 (dd, J=8.5, 1.4 Hz, 1H), 7.75 (dd, J=8.5, 4.4 Hz, 1H), 4.24 (qd, J=6.8, 4.8 Hz, 1H), 3.93 (dt, J=8.3, 6.5 Hz, 1H), 3.84-3.68 (m, 3H), 2.16-1.82 (m, 3H), 1.71 (ddt, J=11.6, 8.0, 6.5 Hz, 1H). ¹⁹F NMR (376 MHz, Methanol-d4) δ −77.50. MS (m/z) 246.1 [M+H]⁺.

### Example 17

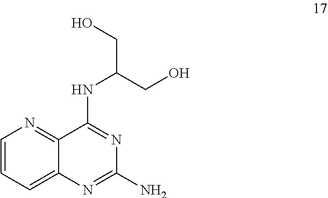 [SMILES: Nc1nc(NC(CO)CO)c2ncccc2n1]

Compound 17, 2-((2-aminopyrido[3,2-d]pyrimidin-4-yl)amino)propane-1,3-diol, was prepared following the procedure for compound 1B reported above, instead replacing butan-1-amine with 2-aminopropane-1,3-diol to yield the title compound (17) as its TFA salt. ¹H NMR (400 MHz, Methanol-d4) δ 8.64 (dd, J=4.4, 1.4 Hz, 1H), 7.85 (dd, J=8.5, 1.4 Hz, 1H), 7.77 (dd, J=8.5, 4.4 Hz, 1H), 4.54 (p, J=5.5 Hz, 1H), 3.84 (d, J=5.5 Hz, 4H). ¹⁹F NMR (376 MHz, Methanol-d4) δ −77.66. MS (m/z) 236.1 [M+H]⁺.

### Example 18

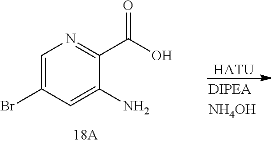

Synthesis of 3-amino-5-bromopicolinamide (18B): To a solution of 3-amino-5-bromopicolinic acid 18A (300 mg, 1.38 mmol, 1 equiv.) in DMF (11 ml, 0.1 M) was added HATU (598 mg, 1.57 mmol, 1.1 equiv.) followed by DIPEA (0.48 mL, 2.76 mmol, 2 equiv.) and ammonium hydroxide (0.8 mL, 5.55 mmol, 4 equiv.). The mixture was allowed to stir overnight. Water (50 mL) was added and the mixture then extracted with EtOAc (3 times). The organic layer was separated, dried over Na₂SO₄, filtered and concentrated under reduced pressure. The product (18B) was obtained after flash chromatography. MS (m/z): 216.8 [M+H]⁺

Synthesis of 2-amino-7-bromopyrido[3,2-d]pyrimidin-4-ol (18C): To a flask containing 3-amino-5-bromopicolinamide (18B) (205 mg, 0.1 mmol, 1 equiv.) was added chloroformamidine hydrochloride (140 mg, 1.3 equiv.). The mixture was heated to 165° C. overnight. It was allowed to cool to room temperature, then filtered and washed with water and ethyl ether. The residue was allowed to air dry to furnish 2-amino-7-bromopyrido[3,2-d]pyrimidin-4-ol (1C) which was used without further purification. MS (m/z): 239.9 [M+H]⁺

Synthesis of N-(7-bromo-4-hydroxypyrido[3,2-d]pyrimidin-2-yl)acetamide (18D): To a flask containing 2-amino-7-bromopyrido[3,2-d]pyrimidin-4-ol (1C) (155 mg, 0.64 mmol, 1 equiv.) was added acetic anhydride (3 mL). The mixture was heated to 115° C. for 4 hrs. It was concentrated under reduced pressure. It was filtered and washed with diethyl ether and hexane and allowed to air dry to obtain N-(7-bromo-4-hydroxypyrido[3,2-d]pyrimidin-2-yl)acetamide (18D). MS (m/z): 282.9 [M+H].⁺

Synthesis of N-(7-bromo-4-chloropyrido[3,2-d]pyrimidin-2-yl)acetamide (18E): Into a solution of N-(7-bromo-4-hydroxypyrido[3,2-d]pyrimidin-2-yl)acetamide (18D) (200 mg, 0.71 mmol, 1 equiv.) was added acetonitrile (2 ml) and POCl₃ (1 ml) followed by DIPEA (0.12 mL, 0.71 mmol, 1 equiv.). The mixture was refluxed for 6 hours. The mixture was concentrated under reduced pressure. To it was added water (20 mL) then extracted with EtOAc (3 times). The organic layer was separated, dried over Na₂SO₄, filtered and concentrated under reduced pressure to afford the title product N-(7-bromo-4-chloropyrido[3,2-d]pyrimidin-2-yl)acetamide (18E). MS (m/z): 298.9 [M+H].⁺

Synthesis of (S)-2-((2-amino-7-bromopyrido[3,2-d]pyrimidin-4-yl)amino)pentan-1-ol (18F): To a solution of N-(7-bromo-4-chloropyrido[3,2-d]pyrimidin-2-yl)acetamide (18E) (215 mg, 0.71 mmol, 1 equiv.) was added DMF (1.5 ml) followed by DIPEA (0.38 mL, 2.1 mmol, 3 equiv.) and (S)-(+)-2-Amino-1-pentanol (55 mg, 3.6 mmol, 5 equiv.). The reaction was allowed to stir overnight. It was concentrated under reduced pressure and purified by reverse phase HPLC to furnish the title compound (18F) as its TFA salt. ¹H NMR (400 MHz, Methanol-d4) δ 8.41 (d, J=2.0 Hz, 1H), 7.83 (d, J=2.0 Hz, 1H), 4.34 (dd, J=8.5, 5.4 Hz, 1H), 3.65-3.53 (m, 3H), 1.67-1.49 (m, 3H), 1.41-1.24 (m, 3H), 0.86 (t, J=7.4 Hz, 5H). ¹⁹F NMR (377 MHz, CD₃OD) δ −77.52. MS (m/z): 368.2 [M+H].

### Example 19

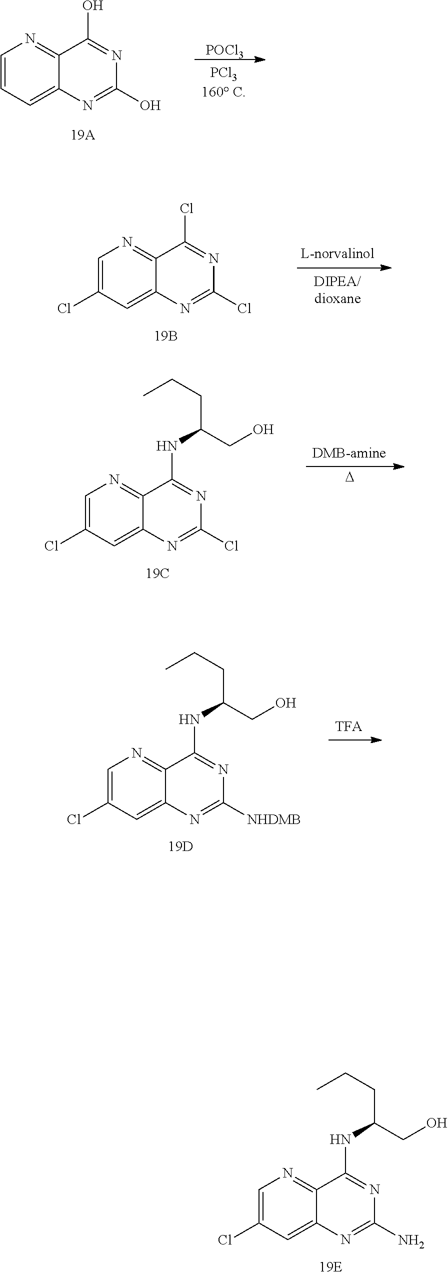

Synthesis of 2,4,7-trichloropyrido[3,2-d]pyrimidine (19B): Into a microwave vial was added pyrido[3,2-d]pyrimidine-2,4-diol (19A) (200 mg, 1.2 mmol, 1 equiv.) is added POCl₃ (2.5 mL) and PCl₅ (1.53 g, 7.4 mmol, 6 equiv.). The mixture was heated to 160° C. for 3 hr in microwave reactor. The reaction mixture was concentrated under reduced pressure and partitioned between EtOAc and H₂O. The organics were separated, dried, and removed in vacuo. The residue purified by column chromatography on silica to provide the title compound. MS (m/z): 236.6 [M+H]⁺.

Synthesis of (S)-2-((2,7-dichloropyrido[3,2-d]pyrimidin-4-yl)amino)pentan-1-ol (19C): To a solution of 2,4,7-trichloropyrido[3,2-d]pyrimidine (19B) (160 mg, 0.68 mmol, 1 equiv.) was added dioxane (4 ml) followed by DIPEA (0.18 mL, 1.2 mmol, 1.5 equiv.) and (S)-(+)-2-Amino-1-pentanol (85 mg, 0.82 mmol, 1.1 equiv.). The reaction was allowed to stir for an hr. It was concentrated under reduced pressure and used as is to provide the title compound. MS (m/z): 301.1 [M+H]⁺.

Synthesis of (S)-2-((7-chloro-2-((2,4-dimethoxybenzyl)amino)pyrido[3,2-d]pyrimidin-4-yl)amino)pentan-1-ol (19D): To a solution of (R)-2-((2,7-dichloropyrido[3,2-d]pyrimidin-4-yl)amino)pentan-1-ol (19C) (206 mg, 0.68 mmol, 1 equiv.) was added dioxane (4 ml) followed by DIPEA (0.24 mL, 1.4 mmol, 2 equiv.) and 2,4-demethoxybenzylamine (0.30 mL, 2.0 mmol, 3 equiv.). The reaction was allowed heated at 120° C. overnight. The reaction mixture was partitioned between EtOAc and H₂O. The organics were separated, dried, and removed in vacuo. The residue purified by column chromatography on silica to provide the title compound. MS (m/z): 432.2 [M+H].⁺

Synthesis of (S)-2-((2-amino-7-chloropyrido[3,2-d]pyrimidin-4-yl)amino)pentan-1-ol (19E): Into a solution of (S)-2-((7-chloro-2-((2,4-dimethoxybenzyl)amino)pyrido[3,2-d]pyrimidin-4-yl)amino)pentan-1-ol (19D) (35 mg, 0.08 mmol, 1 equiv.) was added DCM (2 mL) and TFA (0.5 mL). After 3 hours the reaction mixture was concentrated under reduced pressure and purified by reverse phase HPLC to furnish the title compound (19E) as its TFA salt. ¹H NMR (400 MHz, Methanol-d4) δ 8.48 (d, J=2.0 Hz, 1H), 7.78 (d, J=2.1 Hz, 1H), 4.48 (dd, J=8.6, 5.3 Hz, 1H), 3.93-3.74 (m, 2H), 3.71 (d, J=5.2 Hz, 3H), 1.77-1.57 (m, 2H), 1.50-1.36 (m, 1H), 1.28 (s, 2H), 0.97 (t, J=7.4 Hz, 4H). ¹⁹F NMR (377 MHz, Methanol-d4) δ −77.59 (d, J=80.2 Hz). MS (m/z): 282.1 [M+H].⁺

### General Scheme for Examples 20-22

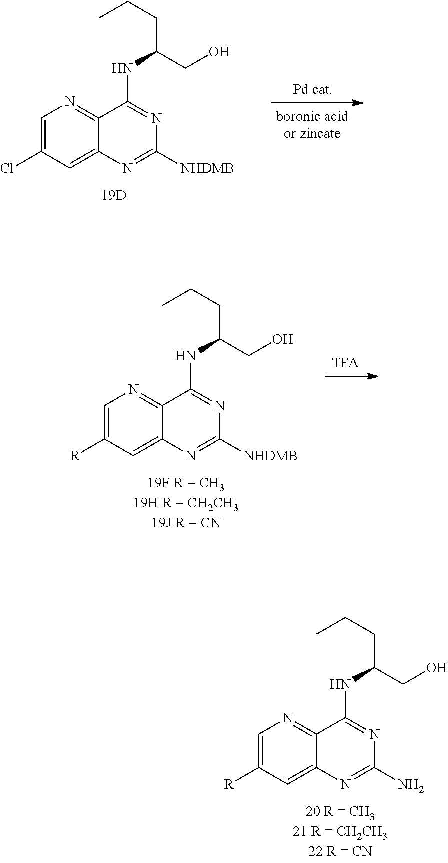

### Example 20

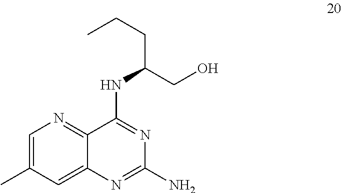 [SMILES: CCC[C@@H](CO)Nc1nc(N)nc2cc(C)cnc12]

Synthesis of(S)-2-((2-((2,4-dimethoxybenzyl)amino)-7-methylpyrido[3,2-d]pyrimidin-4-yl)amino)pentan-1-ol (19F): Into a vial containing (S)-2-((7-chloro-2-((2,4-dimethoxybenzyl)amino)pyrido[3,2-d]pyrimidin-4-yl)amino)pentan-1-ol (19D) (25 mg, 0.06 mmol, 1 equiv.) was added methylboronic acid (8 mg, 0.14 mmol, 2.5 equiv.), potassium phosphate tribasic (37 mg, 0.17 mmol, 3 equiv.), palladium(0)-tetrakis(triphenylphosphine) (7 mg, 0.006 mmol, 0.1 equiv.) along with dioxane (2 mL) and water (2 mL). The mixture is heated to 150° C. for 1 hr in a microwave reactor. The reaction mixture was partitioned between EtOAc and H₂O. The organics were separated, dried, and removed in vacuo to furnish the title compound which was used directly. MS (m/z): 474.3 [M+H].⁺

Synthesis of (S)-2-((2-amino-7-methylpyrido[3,2-d]pyrimidin-4-yl)amino)pentan-1-ol (20): Into the a flask containing 19F was added THF (2 mL), water (2 mL) followed by 2,3-dichloro-5,6-dicyanobenzoquinone (26 mg, 20.11 mmol, 2 equiv.) After stirring overnight, the reaction mixture was partitioned between EtOAc and H₂O. The organics were separated, dried, and removed in vacuo. Purification was carried out using flash column chromatography to furnish the title compound (20). ¹H NMR (400 MHz, Methanol-d4) δ 8.35 (d, J=1.1 Hz, 1H), 7.49 (s, 1H), 4.54-4.34 (m, 1H), 3.70 (d, J=5.0 Hz, 2H), 1.84-1.61 (m, 2H), 1.56-1.35 (m, 2H), 0.97 (t, J=7.3 Hz, 3H). MS (m/z): 262.1 [M+H].⁺

### Example 21

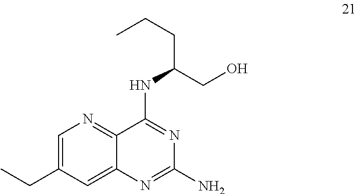 [SMILES: CCC[C@@H](CO)Nc1nc(N)nc2cc(CC)cnc12]

Synthesis of (S)-2-((2-amino-7-ethylpyrido[3,2-d]pyrimidin-4-yl)amino)pentan-1-ol (21) was prepared according to the procedure used for 20, instead using ethylboronic acid in place of methylboronic acid. ¹H NMR (400 MHz, Methanol-d4) δ 8.65-8.30 (m, 1H), 7.62 (s, 1H), 4.61-4.38 (m, 1H), 3.80-3.64 (m, 2H), 2.84 (q, J=7.6 Hz, 2H), 1.71 (tdd, J=8.3, 6.5, 2.2 Hz, 2H), 1.43 (dddd, J=12.4, 7.4, 5.1, 2.5 Hz, 2H), 1.39-1.23 (m, 4H), 0.97 (t, J=7.3 Hz, 3H). MS (m/z): 276.2 [M+H]⁺.

### Example 22

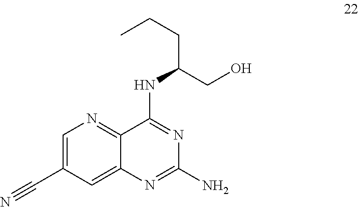 [SMILES: CCC[C@@H](CO)Nc1nc(N)nc2cc(C#N)cnc12]

Synthesis of (S)-2-amino-4-((1-hydroxypentan-2-yl)amino)pyrido[3,2-d]pyrimidine-7-carbonitrile (22) was prepared according to the two step procedure used for 20, instead using Zn(CN)₂ in place of methylboronic acid. ¹H NMR (400 MHz, DMSO-d6) δ 7.93 (d, J=1.7 Hz, 1H), 7.24 (d, J=1.7 Hz, 1H), 2.95-2.68 (m, 3H), 0.76 (d, J=7.3 Hz, 2H), 0.47 (d, J=7.6 Hz, 1H), 0.02 (t, J=7.4 Hz, 4H). MS (m/z): 273.3 [M+H].⁺

### Example 23

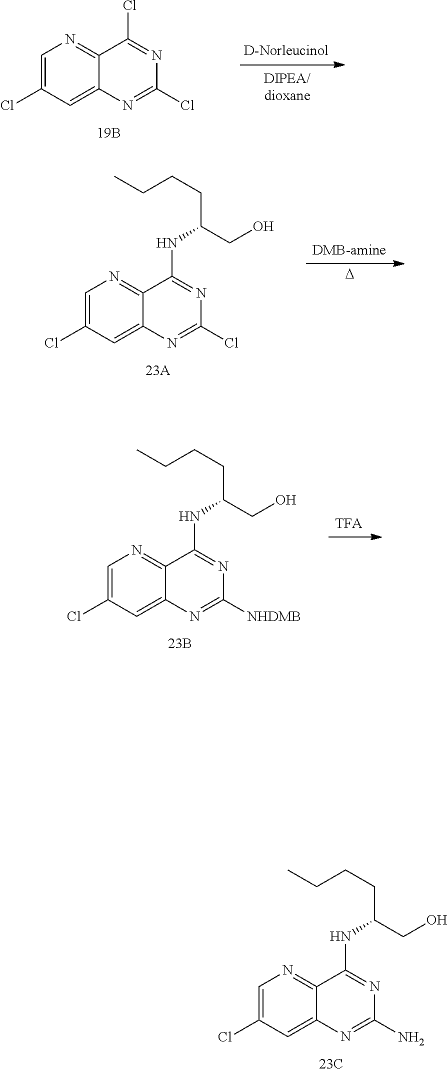

Synthesis of (R)-2-((2,7-dichloropyrido[3,2-d]pyrimidin-4-yl)amino)hexan-1-ol (23A): To a solution of 2,4,7-trichloropyrido[3,2-d]pyrimidine (19B) (45 mg, 0.19 mmol, 1 equiv.) was added dioxane (4 ml) followed by DIPEA (41 μL, 0.23 mmol, 1.2 equiv.) and (R)-(−)-2-Amino-1-hexanol 97% (24.7 mg, 0.21 mmol, 1.1 equiv.). The reaction was allowed to stir for an hr. It was concentrated under reduced pressure and used as is to provide the title compound. MS (m/z): 316.2 [M+H].⁺

Synthesis of (R)-2-((7-chloro-2-((2,4-dimethoxybenzyl)amino)pyrido[3,2-d]pyrimidin-4-yl)amino)hexan-1-ol (23B): To a solution of (R)-2-((2,7-dichloropyrido[3,2-d]pyrimidin-4-yl)amino)hexan-1-ol (23A) (60 mg, 0.19 mmol, 1 equiv.) was added dioxane (4 ml) followed by DIPEA (68 μL, 0.38 mmol, 2 equiv.) and 2,4-demethoxybenzylamine (85 μL, 3.0 mmol, 3 equiv.). The reaction was allowed heated at 120° C. overnight. The reaction mixture partitioned between EtOAc and H₂O. The organics were separated, dried, and removed in vacuo. The residue purified by column chromatography on silica to provide the title compound. MS (m/z): 446.9 [M+H].⁺

Synthesis (R)-2-((2-amino-7-chloropyrido[3,2-d]pyrimidin-4-yl)amino)hexan-1-ol (23C): To a solution of (R)-2-((7-chloro-2-((2,4-dimethoxybenzyl)amino)pyrido[3,2-d]pyrimidin-4-yl)amino)hexan-1-ol (20B) (50 mg, 0.11 mmol, 1 equiv.) was added DCM (2 mL) and TFA (0.5 mL). After 3 hours the reaction mixture was concentrated under reduced pressure and purified by reverse phase HPLC to furnish the title compound (23C) as its TFA salt. ¹H NMR (400 MHz, Methanol-d4) δ 8.60 (d, J=2.1 Hz, 1H), 7.90 (d, J=2.1 Hz, 1H), 4.58-4.44 (m, 1H), 3.79-3.63 (m, 3H), 1.86-1.61 (m, 2H), 1.52-1.24 (m, 5H), 1.01-0.79 (m, 4H). ¹⁹F NMR (377 MHz, Methanol-d4) δ −77.61. MS (m/z): 296.2 [M+H].⁺

### Example 24

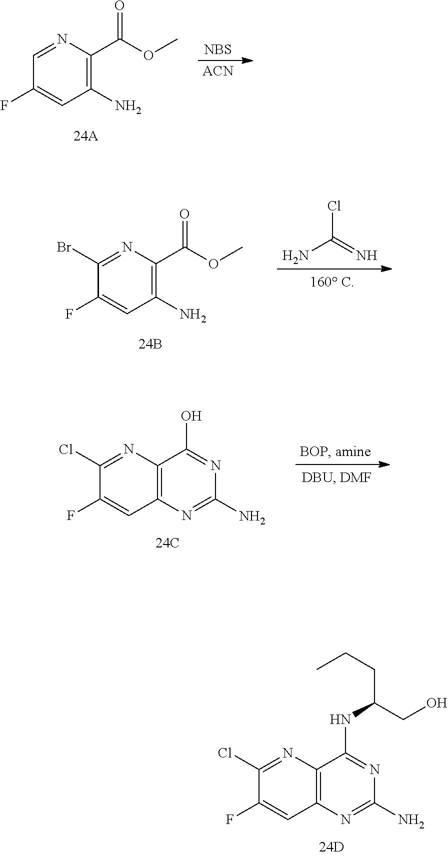

Synthesis of methyl 3-amino-6-bromo-5-fluoropicolinate (24B): To a solution of methyl 3-amino-5-fluoropicolinate (24A) (270 mg, 0.22 mmol, 1 equiv.) was added acetonitrile (5 mL) and N-bromosuccinimide (310 mg, 0.24 mmol, 1.1 equiv.). The reaction was allowed to stir at room temperature overnight. The reaction mixture partitioned between EtOAc and H₂O. The organics were separated, dried, and removed in vacuo. The residue purified by column chromatography on silica to provide the title compound. MS (m/z): 250.2 [M+H].⁺

Synthesis of 2-amino-6-chloro-7-fluoropyrido[3,2-d]pyrimidin-4-ol (24C): To a flask containing methyl 3-amino-6-bromo-5-fluoropicolinate (24B) (200 mg, 0.80 mmol, 1 equiv.) was added chloroformamidine hydrochloride (185 mg, 1.61 mmol, 2 equiv.). The mixture was heated to 165° C. overnight. It was allowed to cool down to room temperature it was filtered and washed with water and ethyl ether. The residue was allowed to air dry to provide the title compound (24C). Approximately, 25% of the product is the corresponding side product 2-amino-6-bromo-7-fluoropyrido[3,2 d]pyrimidin-4-ol. The material was used without further purification. MS (m/z): 260.0 [M+H].⁺

Synthesis of Synthesis of 2-amino-6-chloro-7-fluoropyrido[3,2-d]pyrimidin-4-ol (24D): To a flask 2-amino-6-chloro-7-fluoropyrido[3,2-d]pyrimidin-4-ol (24C) (50 mg, 0.23 mmol, 1 equiv.) is added (Benzotriazol-1-yloxy)tris(dimethylamino)phosphonium hexafluorophosphate 97% (BOP Reagent) (123 mg, 0.28 mmol, 1.2 equiv.), (S)-(+)-2-Amino-1-pentanol, 97% (48 mg, 0.47 mmol, 2 equiv.) and DBU (105 μL, 0.70 mmol, 3 equiv.) and DMF (3 mL). The mixture was allowed to stir at room temperature overnight and purified by reverse phase HPLC to furnish the title compound (24D) as its TFA salt. ¹H NMR (400 MHz, Methanol-d4) δ 7.86-7.63 (m, 1H), 4.64-4.47 (m, 1H), 3.72 (d, J=5.5 Hz, 2H), 1.82-1.61 (m, 3H), 1.56-1.35 (m, 2H), 0.97 (t, J=7.4 Hz, 3H). ¹⁹F NMR (377 MHz, Methanol-d4) δ −77.54, −110.63 (d, J=8.2 Hz). MS (m/z): 300.2 [M+H]⁺.

### Example 25

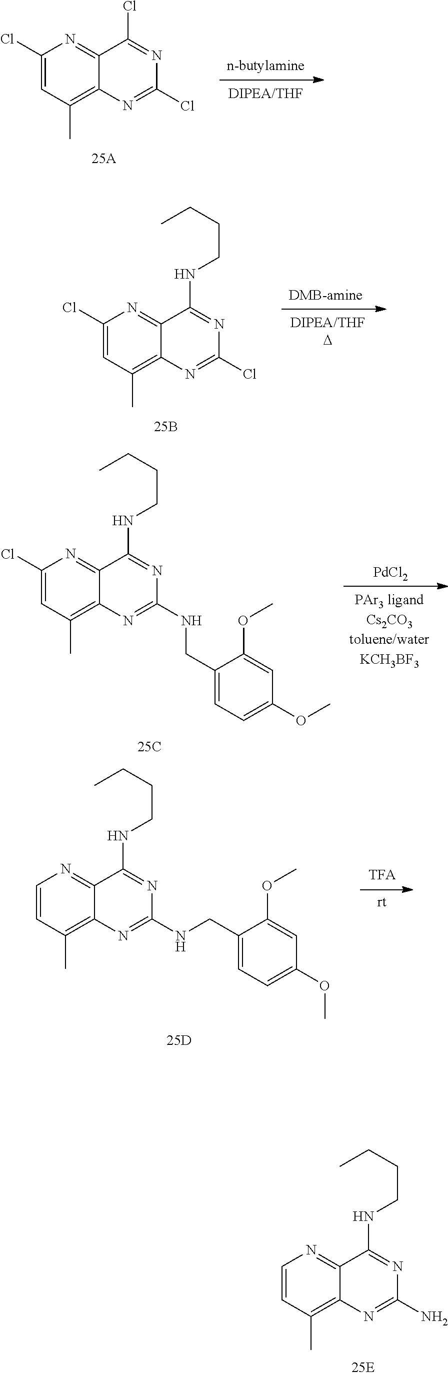

Synthesis of N⁴-butyl-8-methylpyrido[3,2-d]pyrimidine-2,4-diamine (25E). Beginning from intermediate 25A, treatment with 1.05 equiv butan-1-amine in THF/DIPEA at RT gave 25B, which was concentrated to a residue and carried forward directly. Heating with excess 2,4-dimethoxybenzylamine in THF/DIPEA led to compound 25C, with characteristic MS (m/z): 416.2 [M+H].⁺ Following the procedure reported by Hasník et. al in Synthesis, 2009, 1309-1317, instead of the expected 6-methylation via potassium methyl trifluoroborate, protonolysis of the intermediate heteroaryl-Pd complex led mainly to isolation of 25D, and finally to N⁴-butyl-8-methylpyrido[3,2-d]pyrimidine-2,4-diamine 25E upon treatment of 25D in excess TFA and final purification via HPLC to provide the title compound (25E) as its TFA salt. ¹H NMR (400 MHz, Methanol-d4) δ 8.48 (d, J=1.1 Hz, 1H), 7.61 (d, J=1.1 Hz, 1H), 3.67 (d, J=7.2 Hz, 2H), 2.52 (s, 3H), 1.75-1.68 (m, 2H), 1.46-1.35 (m, 2H), 0.98 (t, J=7.3 Hz, 3H). ¹⁹F NMR (377 MHz, Methanol-d4) δ −77.6. MS (m/z): 232.1 [M+H].⁺

### Example 26

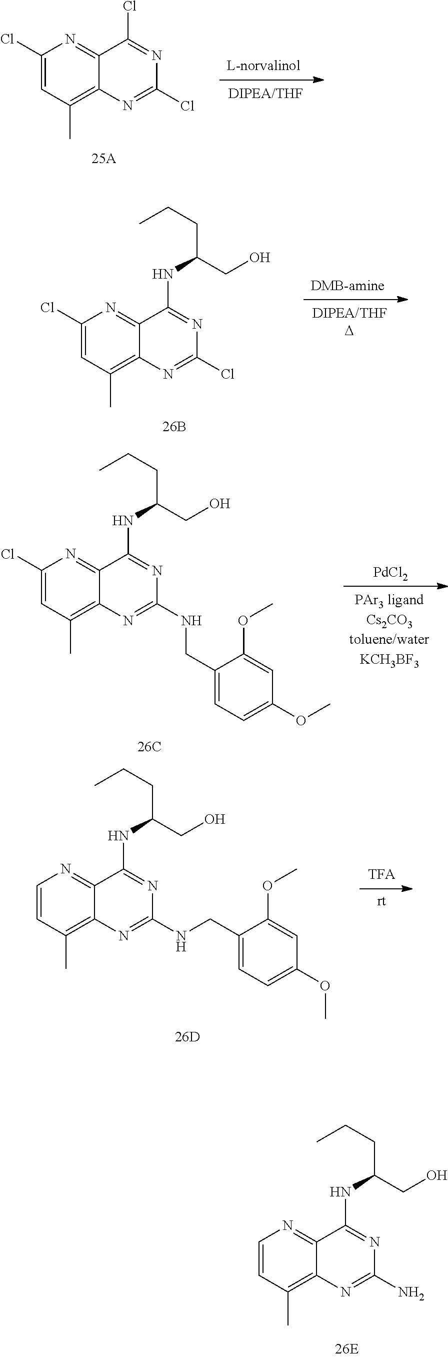 [SMILES: Cc1cc(Cl)nc2c(Cl)nc(Cl)nc12 ; CCC[C@@H](CO)Nc1nc(Cl)nc2c(C)cc(Cl)nc12 ; CCC[C@@H](CO)Nc1nc(NCc2ccc(OC)cc2OC)nc2c(C)cc(Cl)nc12 ; "PdCl2" ; CCC[C@@H](CO)Nc1nc(NCc2ccc(OC)cc2OC)nc2c(C)ccnc12 ; CCC[C@@H](CO)Nc1nc(N)nc2c(C)ccnc12]

Synthesis of (S)-2-((2-amino-8-methylpyrido[3,2-d]pyrimidin-4-yl)amino)pentan-1-ol (26E): Beginning from intermediate 25A and following the synthetic sequence reported above for the synthesis of 25E, but instead using L-norvalinol in place of butan-1-amine, 26E was obtained as its TFA salt. ¹H NMR (400 MHz, Methanol-d4) δ 8.50 (d, J=4.6 Hz, 1H), 7.63 (dq, J=4.5, 0.8 Hz, 1H), 4.60-4.49 (m, 1H), 3.78-3.70 (m, 2H), 2.53 (s, 3H), 1.81-1.64 (m, 2H), 1.52-1.34 (m, 2H), 0.97 (t, J=7.3 Hz, 3H). ¹⁹F NMR (377 MHz, Methanol-d4) δ −77.7. MS (m/z): 262.2 [M+H]⁺

### Example 27

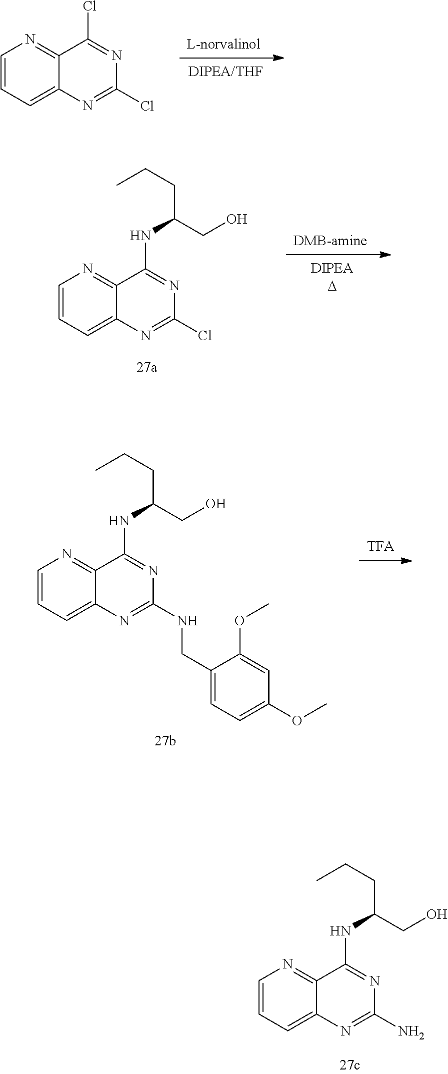 [SMILES: Clc1nc(Cl)c2ncccc2n1 ; CCC[C@@H](CO)Nc1nc(Cl)nc2cccnc12 ; CCC[C@@H](CO)Nc1nc(NCc2ccc(OC)cc2OC)nc2cccnc12 ; CCC[C@@H](CO)Nc1nc(N)nc2cccnc12]

Synthesis of (S)-2-((2-chloropyrido[3,2-d]pyrimidin-4-yl)amino)pentan-1-ol (27C): To a solution of 2,4-dichloropyrido[3,2-d]pyrimidine (160 mg, 0.68 mmol, 1 equiv.) was added THF (4 ml) followed by DIPEA (0.18 mL, 1.2 mmol, 1.5 equiv.) and (S)-(+)-2-amino-1-pentanol (85 mg, 0.82 mmol, 1.1 equiv.). The reaction was allowed to stir for 1 h. The reaction was concentrated under reduced pressure and used as is to provide 27A. MS (m/z): 267.1 [M+H].⁺

Synthesis of (S)-2-((2-((2,4-dimethoxybenzyl)amino)pyrido[3,2-d]pyrimidin-4-yl)amino)pentan-1-ol (27B): To a solution of (S)-2-((2-chloropyrido[3,2-d]pyrimidin-4-yl)amino)pentan-1-ol (27A) (206 mg, 0.68 mmol, 1 equiv.) was added is added THF (4 ml) followed by DIPEA (0.24 mL, 1.4 mmol, 2 equiv.) and 2,4-dimethoxybenzylamine (0.30 mL, 2.0 mmol, 3 equiv.). The reaction was heated at 135C via microwave reactor for 30 minutes. The reaction mixture was partitioned between EtOAc and H₂O. The organics were separated, dried, and removed in vacuo. The residue was purified by column chromatography on silica to provide 27B. MS (m/z): 398.2 [M+H].⁺

Synthesis of (S)-2-((2-amino-[3,2-d]pyrimidin-4-yl)amino)pentan-1-ol (27C): Into a solution of (S)-2-((2-((2,4-dimethoxybenzyl)amino)pyrido[3,2-d]pyrimidin-4-yl)amino)pentan-1-ol (27B) (35 mg, 0.08 mmol, 1 equiv.) was added DCM (2 mL) and TFA (0.5 mL). After 3 hours the reaction mixture was concentrated under reduced pressure and purified by reverse phase HPLC to furnish the title compound (27C) as its TFA salt. ¹H NMR (400 MHz, Methanol-d4) δ 8.65 (dd, J=4.3, 1.5 Hz, 1H), 7.85-7.73 (m, 2H), 4.55 (s, 1H), 3.76-3.70 (m, 2H), 1.77-1.66 (m, 2H), 1.44 (td, J=7.3, 4.2 Hz, 2H), 0.98 (t, J=7.4 Hz, 3H). ¹⁹F NMR (377 MHz, Methanol-d4) δ −77.6. MS (m/z): 248.2 [M+H]⁺

### Example 28

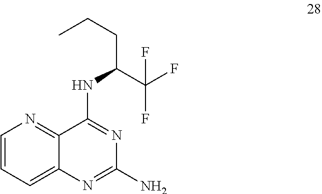 [SMILES: CCC[C@H](Nc1nc(N)nc2cccnc12)C(F)(F)F]

Following the general procedure described above for the synthesis of 1B, 2,4-dichloropyrido[3,2-d]pyrimidine was instead reacted with 1.1 equiv (S)-1,1,1-trifluoropentan-2-amine in place of 1-butan-amine and then carried through the steps as reported above in Example 1 to provide (S)—N⁴-(1,1,1-trifluoropentan-2-yl)pyrido[3,2-d]pyrimidine-2,4-diamine (28). ¹H NMR (400 MHz, DMSO-d6) δ 9.87 (s, 1H), 8.67 (dd, J=4.4, 1.5 Hz, 1H), 7.95-7.81 (m, 2H), 5.13 (t, J=8.9 Hz, 1H), 2.21-2.10 (m, 1H), 1.74 (dd, J=12.1, 7.1 Hz, 1H), 1.44-1.36 (m, 1H), 1.27 (dq, J=13.7, 7.1 Hz, 1H), 0.89 (t, J=7.3 Hz, 3H). ¹⁹F NMR (377 MHz, Methanol-d4) δ −73.9, −74.1. MS (m/z): 286.1 [M+H]⁺.

### Example 29

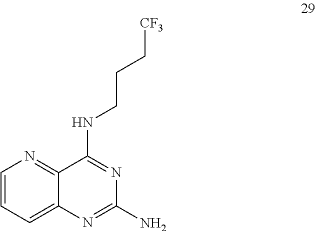 [SMILES: Nc1nc(NCCCC(F)(F)F)c2ncccc2n1]

Following the general procedure described above for the synthesis of 1B, 2,4-dichloropyrido[3,2-d]pyrimidine was instead reacted with 1.1 equiv 4,4,4-trifluorobutylamine in place of 1-butan-amine and then carried through the steps as reported above for Example 1 to provide N⁴-(4,4,4-trifluorobutyl)pyrido[3,2-d]pyrimidine-2,4-diamine (29) after HPLC purification as its TFA salt. ¹H NMR (400 MHz, DMSO-d6) δ 9.74 (t, J=6.0 Hz, 1H), 8.63 (dd, J=4.4, 1.4 Hz, 1H), 8.18-7.50 (m, 2H), 3.62 (q, J=6.7 Hz, 1H), 2.39-2.27 (m, 1H), 1.93-1.84 (m, 1H). ¹⁹F NMR (377 MHz, Methanol-d4) δ −65.5, 75.6. MS (m/z): 272.1 [M+H]⁺

### Example 30

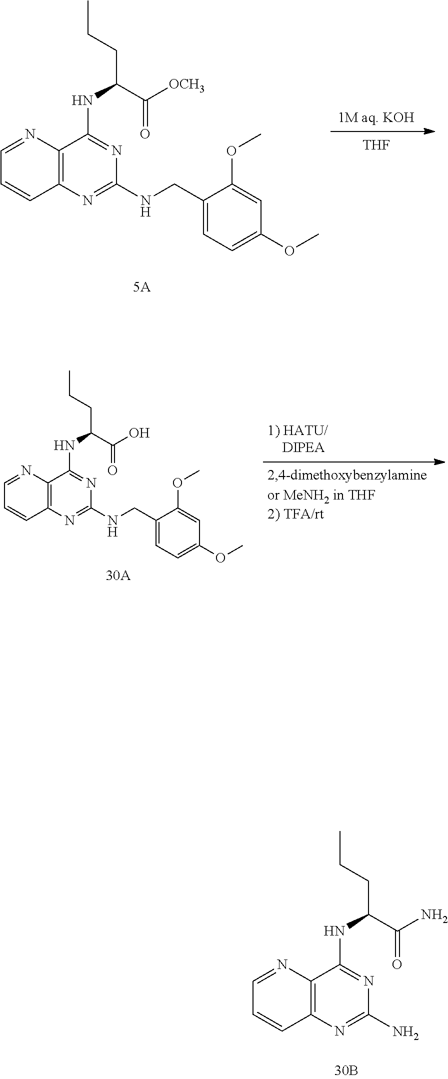 [SMILES: CCC[C@H](Nc1nc(NCc2ccc(OC)cc2OC)nc2cccnc12)C(=O)OC ; CCC[C@H](Nc1nc(NCc2ccc(OC)cc2OC)nc2cccnc12)C(=O)O ; "THF" ; CCC[C@H](Nc1nc(N)nc2cccnc12)C(N)=O]

Synthesis of (S)-2-((2-aminopyrido[3,2-d]pyrimidin-4-yl)amino)pentanamide (30B). Beginning from 50 mg of the intermediate compound 5A previously described above, treatment with 1 equiv. aq. KOH in THF/MEOH (4 mL) for 1 h gave, upon removal of solvent, intermediate 30A, MS (m/z): 399.1 [M+H]⁺ 0.30 A was treated with 1.5 equiv HATU and 3 equiv DIPEA in 2 mL DMF, with quenching by excess 2,4-dimethoxybenzylamine (DMB) to provide the intermediate amide. After global DMB removal via TFA treatment, HPLC purification of the product residue provided title compound 30B as its TFA salt. ¹H NMR (400 MHz, Methanol-d4) δ 8.67 (ddd, J=9.2, 4.3, 1.5 Hz, 1H), 7.89-7.73 (m, 2H), 4.00-3.59 (m, 1H), 2.81 (s, 2H), 2.22-1.79 (m, 2H), 1.48 (tt, J=9.8, 7.4 Hz, 2H), 0.99 (t, J=7.4 Hz, 3H). ¹⁹F NMR (377 MHz, Methanol-d4) δ −77.6. MS (m/z): 261.1 [M+H]⁺.

### Example 31

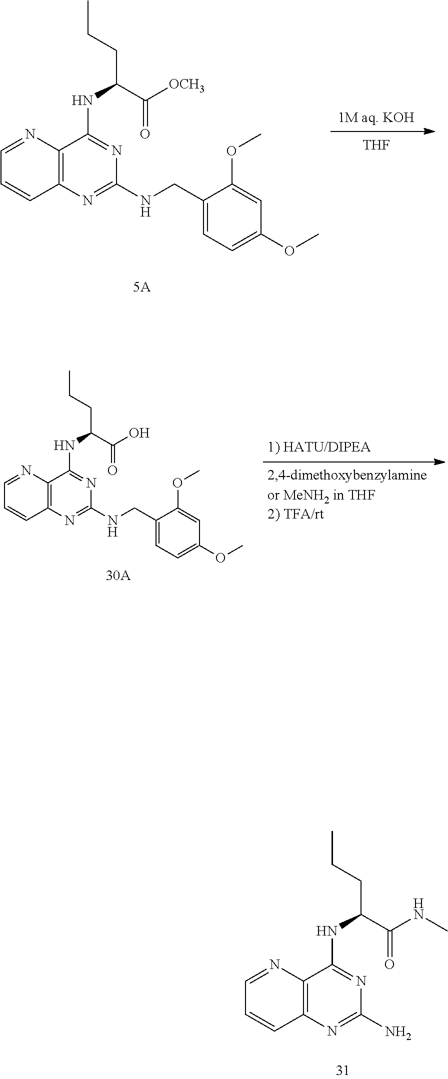 [SMILES: CCC[C@H](Nc1nc(NCc2ccc(OC)cc2OC)nc2cccnc12)C(=O)OC ; CCC[C@H](Nc1nc(NCc2ccc(OC)cc2OC)nc2cccnc12)C(=O)O ; "THF" ; CCC[C@H](Nc1nc(N)nc2cccnc12)C(=O)NC]

Synthesis of (S)-2-((2-aminopyrido[3,2-d]pyrimidin-4-yl)amino)-N-methylpentanamide (31). 50 mg of 30A was treated with 1.5 equiv HATU and 3 equiv DIPEA in 2 mL DMF, with quenching by 1.0 M methylamine in THF to provide the intermediate methylamide. After standard DMB removal via TFA treatment, HPLC purification of the product residue provided title compound 31 as its TFA salt. ¹H NMR (400 MHz, Methanol-d₄) δ 8.68 (dd, J=4.3, 1.5 Hz, 1H), 7.89-7.76 (m, 2H), 4.85 (m, 1H), 2.76 (s, 3H), 2.08-1.85 (m, 2H), 1.45 (dddd, J=16.5, 13.8, 11.5, 7.4 Hz, 2H), 0.98 (t, J=7.4 Hz, 3H). ¹⁹F NMR (377 MHz, Methanol-d4) δ −77.9. MS (m/z): 275.1 [M+H]⁺

### Example 32

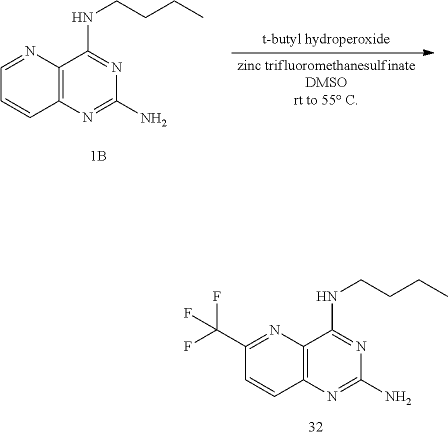 [SMILES: CCCCNc1nc(N)nc2cccnc12 ; CCCCNc1nc(N)nc2ccc(C(F)(F)F)nc12]

Synthesis of N⁴-butyl-6-(trifluoromethyl)pyrido[3,2-d]pyrimidine-2,4-diamine (32). Beginning from 10 mg compound 1B, the synthesis of which is reported in Example 1, and proceeding with chemistry described by Yining et al. in PNAS, 2011, 108, 14411, 1B was heated at 55° C. in DMSO in the presence of 10 equivalents of zinc trifluoromethane sulfinate and 10 equiv t-butylhydroperoxide 70% aq. solution. After 24 h, the reaction mixture was injected directly onto HPLC for final purification to provide the title compound (32) as the corresponding TFA salt. ¹H NMR (400 MHz, Methanol-d₄) δ 8.15 (d, J=8.7 Hz, 1H), 8.01 (dd, J=8.8, 0.8 Hz, 1H), 3.82-3.56 (m, 2H), 1.83-1.61 (m, 2H), 1.58-1.31 (m, 2H), 0.99 (t, J=7.4 Hz, 3H). ¹⁹F NMR (377 MHz, Methanol-d₄) δ −69.0, −77.6. MS (m/z): 286.1 [M+H]⁺.

### Example 33

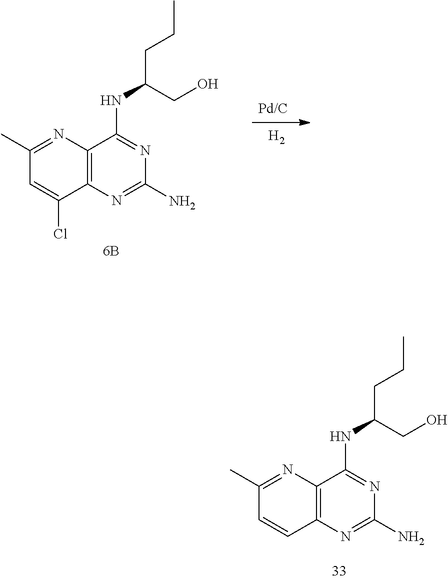 [SMILES: CCC[C@@H](CO)Nc1nc(N)nc2c(Cl)cc(C)nc12 ; "H2" ; CCC[C@@H](CO)Nc1nc(N)nc2ccc(C)nc12]

Synthesis of (S)-2-((2-amino-6-methylpyrido[3,2-d]pyrimidin-4-yl)amino)pentan-1-ol (33). 50 mg compound 6B, (0.11 mmol, 1 equiv) in 10 mL (1:1 EtOH/EtOAc) was reacted with 28 mg 5% Pd/C at 70° C. under 1 atm H₂. After overnight, the reaction was filtered to remove catalyst and the product chromatographed on silica gel, eluting at 25% MeOH/75% EtOAc to provide the title compound (33) as its TFA salt. ¹H NMR (400 MHz, Methanol-d₄) δ 7.74 (d, J=8.6 Hz, 1H), 7.65 (d, J=8.6 Hz, 1H), 4.54 (ddd, J=12.4, 7.3, 5.2 Hz, 1H), 3.75 (d, J=5.2 Hz, 2H), 2.65 (s, 3H), 1.73 (q, J=7.5 Hz, 2H), 1.44 (ddt, J=14.6, 7.4, 4.2 Hz, 2H), 0.98 (t, J=7.3 Hz, 3H). ¹⁹F NMR (377 MHz, Methanol-d4) δ −77.7. MS (m/z) 262.14 [M+H]⁺.

### Example 34

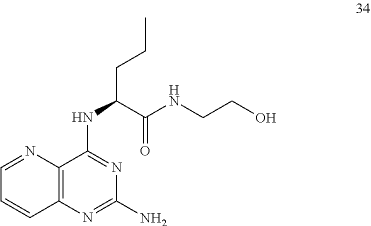 [SMILES: CCC[C@H](Nc1nc(N)nc2cccnc12)C(=O)NCCO]

Synthesis of (S)-2-((2-aminopyrido[3,2-d]pyrimidin-4-yl)amino)-N-(2-hydroxyethyl)pentanamide (34): The title compound was synthesized in a similar fashion to 30B as reported in Example 30, instead replacing methanolic ammonia with ethanolamine to provide the title compound (34) as its TFA salt. ¹H NMR (400 MHz, Methanol-d₄) δ 8.68 (dd, J=4.3, 1.5 Hz, 1H), 7.86 (dd, J=8.6, 1.5 Hz, 1H), 7.80 (dd, J=8.5, 4.4 Hz, 1H), 4.88 (d, J=5.5 Hz, 1H), 3.27-3.22 (m, 2H), 2.11-1.90 (m, 3H), 1.70-1.40 (m, 5H), 1.00 (t, J=7.4 Hz, 3H). ¹⁹F NMR (377 MHz, Methanol-d4) δ −77.5. MS (m/z) 305.21 [M+H]⁺.

### Example 35

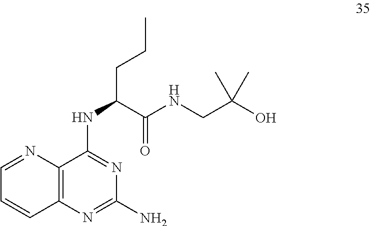 [SMILES: CCC[C@H](Nc1nc(N)nc2cccnc12)C(=O)NCC(C)(C)O]

Synthesis of (S)-2-((2-aminopyrido[3,2-d]pyrimidin-4-yl)amino)-N-(2-hydroxy-2-methylpropyl)pentanamide (35): Compound (35) was synthesized in a similar fashion to 30B as reported in Example 30, instead replacing methanolic ammonia with 1-amino-2-methyl-2-propanol to provide the title compound (35) as its TFA salt. ¹H NMR (400 MHz, Methanol-d₄) δ 8.67 (dd, J=4.4, 1.4 Hz, 1H), 7.87 (dd, J=8.5, 1.4 Hz, 1H), 7.79 (dd, J=8.5, 4.4 Hz, 1H), 4.84-4.78 (m, 1H), 3.61 (td, J=5.9, 5.5, 1.5 Hz, 2H), 2.09-1.85 (m, 2H), 1.48 (dddd, J=18.0, 13.7, 9.7, 7.3 Hz, 2H), 1.29 (s, 6H), 0.99 (t, J=7.4 Hz, 3H). ¹⁹F NMR (377 MHz, Methanol-d4) δ −77.5. MS (m/z) 333.25 [M+H]⁺

### Example 36

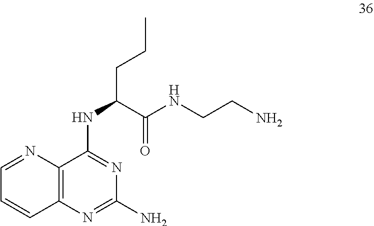 [SMILES: CCC[C@H](Nc1nc(N)nc2cccnc12)C(=O)NCCN]

Synthesis of (S)—N-(2-aminoethyl)-2-((2-aminopyrido[3,2-d]pyrimidin-4-yl)amino)pentanamide (36): Compound 36 was synthesized in a similar fashion to 30B, instead replacing methanolic ammonia with N-Boc-ethylenediamine. Global deprotection with TFA furnished the title compound (36) as its bis-TFA salt. ¹H NMR (400 MHz, Methanol-d₄) δ 8.68 (dd, J=4.4, 1.4 Hz, 1H), 7.88 (dd, J=8.5, 1.4 Hz, 1H), 7.81 (dd, J=8.5, 4.3 Hz, 1H), 4.92 (dd, J=8.6, 5.1 Hz, 1H), 3.56 (ddd, J=13.9, 12.8, 6.7 Hz, 1H), 3.45 (dt, J=14.3, 6.1 Hz, 1H), 3.08 (hept, J=6.4 Hz, 2H), 2.13-2.00 (m, 1H), 2.00-1.85 (m, 1H), 1.55-1.41 (m, 2H), 0.99 (t, J=7.4 Hz, 3H). ¹⁹F NMR (377 MHz, Methanol-d4) δ −77.6. MS (m/z) 304.05 [M+H]⁺.

### Example 37

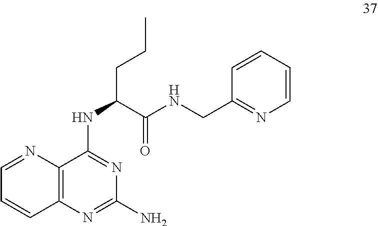 [SMILES: CCC[C@H](Nc1nc(N)nc2cccnc12)C(=O)NCc1ccccn1]

Synthesis of (S)-2-((2-aminopyrido[3,2-d]pyrimidin-4-yl)amino)-N-(pyridin-2-ylmethyl)pentanamide (37): Compound 37 was synthesized in a similar fashion to 30B, instead replacing methanolic ammonia with 2-picolylamine to provide the title compound (37) as the bis TFA salt. ¹H NMR (400 MHz, Methanol-d₄) δ 8.69 (dd, J=4.4, 1.5 Hz, 1H), 8.65-8.62 (m, 1H), 8.22 (td, J=7.8, 1.7 Hz, 1H), 7.88 (dd, J=8.5, 1.4 Hz, 1H), 7.81 (dd, J=8.5, 4.4 Hz, 1H), 7.73 (d, J=8.0 Hz, 1H), 7.67 (dd, J=7.5, 5.7 Hz, 1H), 4.93 (dd, J=8.8, 5.2 Hz, 1H), 4.65 (s, 2H), 2.13-1.94 (m, 3H), 1.57-1.40 (m, 3H), 1.00 (t, J=7.4 Hz, 3H). ¹⁹F NMR (377 MHz, Methanol-d4) δ −77.8. MS (m/z) 352.04 [M+H]⁺.

### Example 38

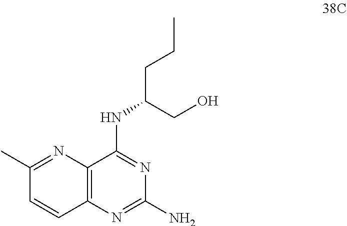 [SMILES: CCC[C@H](CO)Nc1nc(N)nc2ccc(C)nc12]

Synthesis of (R)-2-((8-chloro-2-((2,4-dimethoxybenzyl)amino)-6-methylpyrido[3,2-d]pyrimidin-4-yl)amino)pentan-1-ol (38A): 38A was synthesized in a similar fashion to 6A, instead replacing (S)-norvalinol with (R)-2-aminopentanol and 2,4-dichloropyrido[3,2-d]pyrimidine with 2,4,8-trichloro-6-methylpyrido[3,2-d]pyrimidine. MS (m/z) 446.24 [M+H]⁺.

Synthesis of (R)-2-((2-((2,4-dimethoxybenzyl)amino)-6-methylpyrido[3,2-d]pyrimidin-4-yl)amino)pentan-1-ol (38B): 38B was synthesized in a similar fashion to 6B. MS (m/z) 412.22 [M+H]⁺.

Synthesis of (R)-2-((2-amino-6-methylpyrido[3,2-d]pyrimidin-4-yl)amino)pentan-1-ol (38C): Compound 38C was synthesized in a similar fashion to 33, providing the title compound (38C) as its TFA salt. ¹H NMR (400 MHz, Methanol-d₄) δ 7.69 (d, J=8.5 Hz, 1H), 7.59 (d, J=8.4 Hz, 1H), 4.49 (qd, J=7.9, 6.9, 4.1 Hz, 1H), 3.71 (d, J=5.0 Hz, 2H), 2.60 (s, 3H), 1.68 (q, J=7.5 Hz, 2H), 1.44-1.33 (m, 2H), 0.93 (t, J=7.3 Hz, 3H). ¹⁹F NMR (377 MHz, Methanol-d4) δ −77.3. MS (m/z) 262.15 [M+H]⁺.

### Example 39

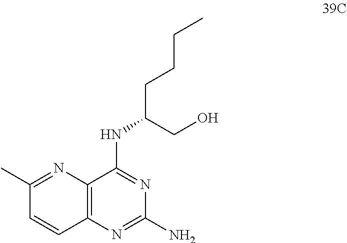 [SMILES: CCCC[C@H](CO)Nc1nc(N)nc2ccc(C)nc12]

Synthesis of (R)-2-((8-chloro-2-((2,4-dimethoxybenzyl)amino)-6-methylpyrido[3,2-d]pyrimidin-4-yl)amino)hexan-1-ol (39A): 39A was synthesized in a similar fashion to 1A, instead replacing butan-1-amine with (R)-2-aminohexanol and 2,4-dichloropyrido[3,2-d]pyrimidine with 2,4,8-trichloro-6-methylpyrido[3,2-d]pyrimidine. MS (m/z) 460.21 [M+H]⁺.

Synthesis of (R)-2-((2-((2,4-dimethoxybenzyl)amino)-6-methylpyrido[3,2-d]pyrimidin-4-yl)amino)hexan-1-ol (39B): 39B was synthesized in a similar fashion to 33. MS (m/z) 426.24 [M+H]⁺.

Synthesis of (R)-2-((2-amino-6-methylpyrido[3,2-d]pyrimidin-4-yl)amino)hexan-1-ol (39C): Compound 39C was synthesized in a similar fashion to 1B to provide the title compound (39C) as its TFA salt. ¹H NMR (400 MHz, Methanol-d₄) δ 7.72 (d, J=8.5 Hz, 1H), 7.62 (d, J=8.4 Hz, 1H), 4.50 (dt, J=8.4, 5.2 Hz, 1H), 3.73 (d, J=5.1 Hz, 2H), 2.63 (s, 3H), 1.80-1.67 (m, 2H), 1.44-1.32 (m, 5H), 0.93-0.86 (m, 3H). ¹⁹F NMR (377 MHz, Methanol-d4) δ −77.3. MS (m/z) 276.17 [M+H]⁺.

### Example 40

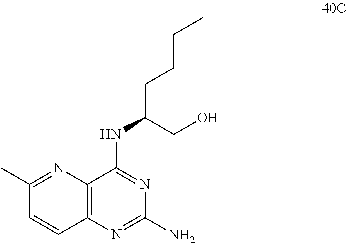 [SMILES: CCCC[C@@H](CO)Nc1nc(N)nc2ccc(C)nc12]

Synthesis of (S)-2-((8-chloro-2-((2,4-dimethoxybenzyl)amino)-6-methylpyrido[3,2-d]pyrimidin-4-yl)amino)hexan-1-ol (40A): 40A was synthesized in a similar fashion to IA, replacing butan-1-amine with (S)-2-aminohexanol and 2,4-dichloropyrido[3,2-d]pyrimidine with 2,4,8-trichloro-6-methylpyrido[3,2-d]pyrimidine. MS (m/z) 460.26 [M+H]⁺.

Synthesis of(S)-2-((2-((2,4-dimethoxybenzyl)amino)-6-methylpyrido[3,2-d]pyrimidin-4-yl)amino)hexan-1-ol (40b): 40b was synthesized in a similar fashion to 33. MS (m/z) 426.24 [M+H]⁺.

Synthesis of (S)-2-((2-amino-6-methylpyrido[3,2-d]pyrimidin-4-yl)amino)hexan-1-ol (40C): Compound 40C was synthesized in a similar fashion to 1B. to provide the title compound (40C) as its TFA salt. ¹H NMR (400 MHz, Methanol-d₄) δ 7.73 (d, J=8.6 Hz, 1H), 7.63 (d, J=8.6 Hz, 1H), 4.51 (dq, J=8.5, 6.1, 5.4 Hz, 1H), 3.75 (d, J=5.2 Hz, 2H), 2.64 (s, 3H), 1.84-1.65 (m, 3H), 1.38 (qd, J=8.0, 6.4, 2.9 Hz, 5H), 0.95-0.87 (m, 4H). ¹⁹F NMR (377 MHz, Methanol-d4) δ −77.6. MS (m/z) 276.16 [M+H]⁺.

### Example 41

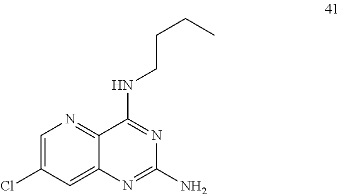 [SMILES: CCCCNc1nc(N)nc2cc(Cl)cnc12]

N⁴-butyl-7-chloropyrido[3,2-d]pyrimidine-2,4-diamine (41). Compound 41 was synthesized following the procedure described above for preparation of 19E, instead reacting intermediate 19B with 1-butan-amine and proceeding with the reported sequence to yield the title compound (41) as the TFA salt after final HPLC purification. ¹H NMR (400 MHz, Methanol-d4) δ 8.56 (d, J=2.1 Hz, 1H), 7.90 (d, J=2.0 Hz, 1H), 3.66 (t, J=7.3 Hz, 2H), 1.76-1.64 (m, 2H), 1.59 (s, 0H), 1.43 (dq, J=14.7, 7.4 Hz, 2H), 0.98 (t, J=7.4 Hz, 3H). ¹⁹F NMR (376 MHz, Methanol-d4) δ −77.55. MS (m/z) 252.2 [M+H]⁺.

### Example 42

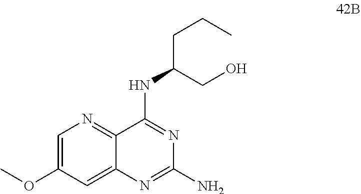 [SMILES: CCC[C@@H](CO)Nc1nc(N)nc2cc(OC)cnc12]

(S)-2-((2-amino-7-methoxypyrido[3,2-d]pyrimidin-4-yl)amino)pentan-1-ol (42B) was prepared according to the following scheme:

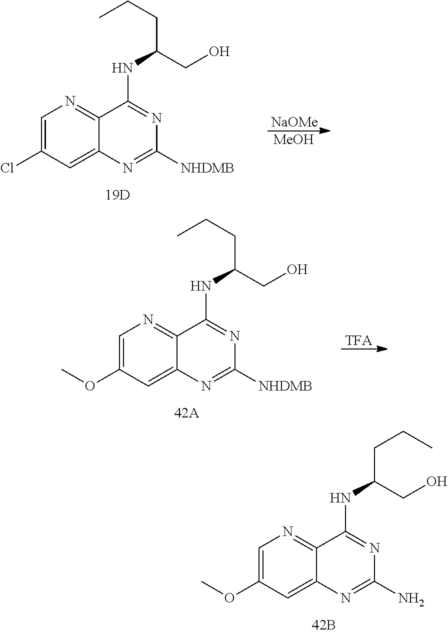

(S)-2-((2-((2,4-dimethoxybenzyl)amino)-7-methoxypyrido[3,2-d]pyrimidin-4-yl)amino)pentan-1-ol (42A): Into a vial containing (S)-2-((7-chloro-2-((2,4-dimethoxybenzyl)amino)pyrido[3,2-d]pyrimidin-4-yl)amino)pentan-1-ol (19D) (50 mg, 0.11 mmol, 1 equiv.) was added NaOMe (65 μL, 1.1 mmol, 10 equiv.) and methanol (2 mL). The mixture was heated to 150° C. for 30 min. in a microwave reactor. The reaction mixture was partitioned between EtOAc and H₂O. The organic layer was separated, dried, and removed in vacuo. The residue was purified by column chromatography on silica to provide the title compound. MS (m/z): 428.2 [M+H].⁺

Compound 42B was synthesized via TFA treatment of 42A to yield the title compound (42B) as the TFA salt after final HPLC purification. ¹H NMR (400 MHz, Methanol-d4) δ 8.32 (d, J=2.5 Hz, 1H), 7.21 (d, J=2.5 Hz, 1H), 4.57-4.45 (m, 1H), 4.00 (s, 3H), 3.77-3.67 (m, 2H), 1.80-1.63 (m, 2H), 1.50-1.39 (m, 2H), 0.97 (t, J=7.4 Hz, 3H). ¹⁹F NMR (377 MHz, Methanol-d4) δ −77.52. MS (m/z) 278.2 [M+H]⁺.

### Example 43

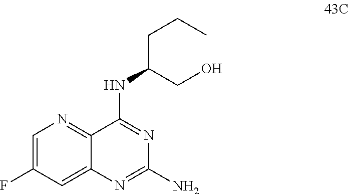 [SMILES: CCC[C@@H](CO)Nc1nc(N)nc2cc(F)cnc12]

Synthesis of (S)-2-((2-amino-7-fluoropyrido[3,2-d]pyrimidin-4-yl)amino)pentan-1-ol (43C):

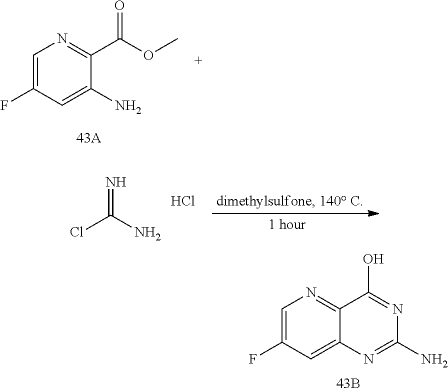

Methyl 3-amino-5-fluoropicolinate (43A) (830 mg, 4.88 mmol), chloroformamidine hydrochloride (1121.64 mg, 9.76 mmol), dimethyl sulfone (4592.09 mg, 48.78 mmol) and a stir bar were charged into a sealed pressure tube and heated to 160° C. for 1 hour. At this time reaction was allowed to cool, 50 mL of water was added and the solution stirred with heating for 30 minutes. Precipitates were filtered off and the mother liquor was purified by reverse phase HPLC using ACN/H₂O with 0.1% TFA as the eluent on a Hydro-RP column with a 2 to 5% ACN gradient. Solvents were removed under reduced pressure and the residue was azeotroped 2× with methanol, 2× with DCM before sonication in ether. Precipitates were filtered and air dried to afford 210 mg (23.9%) of 2-amino-7-fluoropyrido[3,2-d]pyrimidin-4-ol (43B) as a white solid. ¹H NMR (400 MHz, DMSO-d6) δ 8.43 (d, J=2.5 Hz, 1H), 7.48 (dd, J=10.1, 2.5 Hz, 1H), 7.23 (s, 2H). ¹⁹F NMR (376 MHz, DMSO-d6) δ −75.15, −119.96. MS (m/z) 181.0 [M+H]⁺.

Compound 43C was synthesized via a BOP-Cl promoted coupling of 43B with (S)-norvalinol, which provided the title compound (43C) as its TFA salt after final HPLC purification. ¹H NMR (400 MHz, Methanol-d4) δ 8.56 (d, J=2.4 Hz, 1H), 7.61 (dd, J=8.8, 2.5 Hz, 1H), 4.56 (dq, J=12.7, 6.4, 6.0 Hz, 1H), 3.80-3.69 (m, 2H), 1.78 (ddd, J=18.8, 11.4, 3.7 Hz, 2H), 1.53-1.33 (m, 2H), 0.97 (t, J=7.4 Hz, 3H). ¹⁹F NMR (377 MHz, Methanol-d4) δ −77.64, −118.17 (d, J=8.8 Hz). MS (m/z) 266.2 [M+H]⁺.

### Example 44

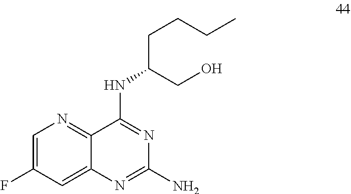 [SMILES: CCCC[C@H](CO)Nc1nc(N)nc2cc(F)cnc12]

(R)-2-((2-amino-7-fluoropyrido[3,2-d]pyrimidin-4-yl)amino)hexan-1-ol (44). Compound 44 was synthesized following the procedure described above for preparation of 43C, instead reacting intermediate 43B with (R)-norleucinol and proceeding with the above reported sequence to yield the title compound (44) as the TFA salt after final HPLC purification. ¹H NMR (400 MHz, Methanol-d4) δ 8.57 (d, J=2.4 Hz, 1H), 7.60 (dd, J=8.8, 2.4 Hz, 1H), 4.53 (dq, J=8.7, 5.6 Hz, 1H), 3.72 (d, J=5.4 Hz, 2H), 1.72 (m, 2H), 1.52-1.28 (m, 4H), 1.04-0.82 (m, 3H). ¹⁹F NMR (377 MHz, Methanol-d4) δ −77.60, −118.13 (d, J=8.6 Hz). MS (m/z) 280.2 [M+H]⁺.

### Example 45

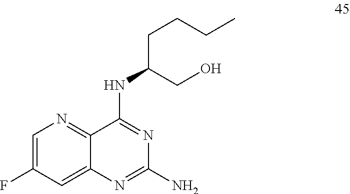 [SMILES: CCCC[C@@H](CO)Nc1nc(N)nc2cc(F)cnc12]

(S)-2-((2-amino-7-fluoropyrido[3,2-d]pyrimidin-4-yl)amino)hexan-1-ol (45). Compound 45 was synthesized following the procedure described above for preparation of 43C, instead reacting intermediate 43B with (S)-norleucinol and proceeding with the above reported sequence to yield the title compound (45) as the TFA salt after final HPLC purification. ¹H NMR (400 MHz, Methanol-d4) δ 8.57 (d, J=2.4 Hz, 1H), 7.60 (dd, J=8.8, 2.4 Hz, 1H), 4.53 (dq, J=8.7, 5.6 Hz, 1H), 3.72 (d, J=5.4 Hz, 2H), 1.72 (m, 2H), 1.52-1.28 (m, 4H), 1.04-0.82 (m, 3H). ¹⁹F NMR (376 MHz, Methanol-d4) δ −77.60, −118.13 (d, J=8.6 Hz). MS (m/z) 280.2 [M+H]⁺.

### Example 46

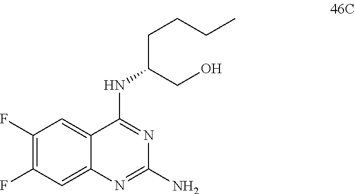 [SMILES: CCCC[C@H](CO)Nc1nc(N)nc2cc(F)c(F)cc12]

Synthesis of (R)-2-((2-amino-6,7-difluoroquinazolin-4-yl)amino)hexan-1-ol (46C):

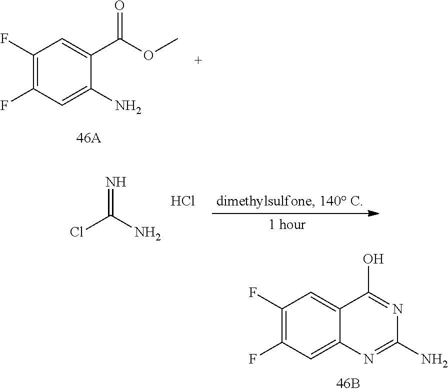

2-amino-6,7-difluoroquinazolin-4-ol (46B) was synthesized following the procedure described above for preparation of 43B, instead reacting intermediate 46A in place of 43A and proceeding with the above reported sequence to yield the title compound (46C) as the TFA salt after final HPLC purification. ¹H NMR (400 MHz, DMSO-d6) ¹H NMR (400 MHz, DMSO-d6) δ 7.83 (t, J=9.7 Hz, 1H), 7.31-7.22 (m, 1H), 7.19 (s, 1H). ¹⁹F NMR (376 MHz, DMSO-d6) δ −74.93, −128.78, −144.35. MS (m/z) 198.0 [M+H]⁺.

Compound (46C) was synthesized via a BOP-Cl promoted coupling of 46B with (R)-norleucinol, which provided the title compound (46C) as its TFA salt after final HPLC purification. ¹H NMR (400 MHz, Methanol-d4) δ 8.29 (dd, J=11.0, 7.9 Hz, 1H), 7.35 (dd, J=10.6, 6.8 Hz, 1H), 4.67-4.53 (m, 1H), 3.80-3.59 (m, 2H), 1.77-1.63 (m, 2H), 1.49-1.30 (m, 4H), 0.91 (td, J=7.0, 6.3, 2.2 Hz, 3H). ¹⁹F NMR (376 MHz, Methanol-d4) δ −77.71, −127.97 (ddd, J=21.5, 10.6, 7.9 Hz), −142.27 (ddd, J=21.4, 11.0, 6.9 Hz). MS (m/z) 297.2 [M+H]⁺.

### Example 47

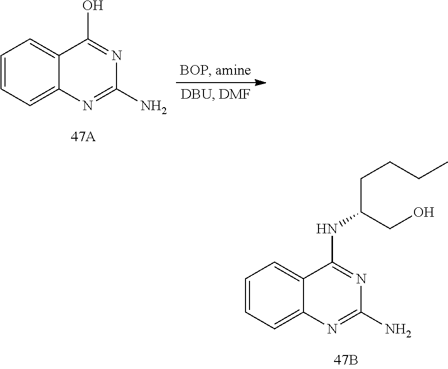 [SMILES: Nc1nc(O)c2ccccc2n1 ; CCCC[C@H](CO)Nc1nc(N)nc2ccccc12]

(R)-2-((2-aminoquinazolin-4-yl)amino)hexan-1-ol (47B) was synthesized via a BOP-Cl promoted coupling of 47A with (R)-norleucinol, which provided the title compound (47B) as its TFA salt after final HPLC purification. ¹H NMR (400 MHz, Methanol-d4) δ 8.22 (ddd, J=8.3, 1.3, 0.6 Hz, 1H), 7.78 (ddd, J=8.4, 7.3, 1.3 Hz, 1H), 7.50-7.33 (m, 2H), 4.71-4.56 (m, 1H), 3.80-3.61 (m, 2H), 1.81-1.64 (m, 2H), 1.47-1.31 (m, 4H), 0.92 (h, J=3.2 Hz, 3H). ¹⁹F NMR (376 MHz, Methanol-d4) δ −77.69. MS (m/z) 261.1 [M+H]⁺.

### Example 48

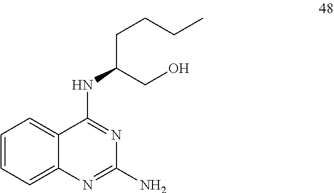 [SMILES: CCCC[C@@H](CO)Nc1nc(N)nc2ccccc12]

Synthesis (S)-2-((2-aminoquinazolin-4-yl)amino)hexan-1-ol (48) was prepared in a similar fashion to 47B, instead using (S)-norleucinol in place of (R)-norleucinol. ¹H NMR (400 MHz, Methanol-d4) δ 8.22 (ddd, J=8.3, 1.3, 0.6 Hz, 1H), 7.78 (ddd, J=8.4, 7.3, 1.3 Hz, 1H), 7.50-7.33 (m, 2H), 4.71-4.56 (m, 1H), 3.80-3.61 (m, 2H), 1.81-1.64 (m, 2H), 1.47-1.31 (m, 4H), 0.92 (h, J=3.2 Hz, 3H). ¹⁹F NMR (376 MHz, Methanol-d4) δ −77.69. MS (m/z) 261.1 [M+H]⁺.

### Example 49

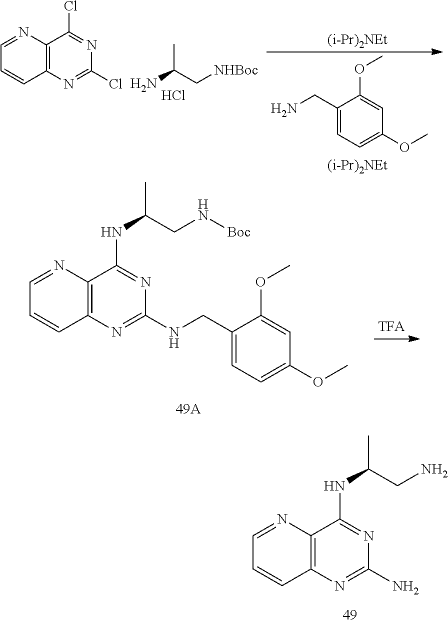

Synthesis of (S)-tert-butyl (2-((2-((2,4-dimethoxybenzyl)amino)pyrido[3,2-d]pyrimidin-4-yl)amino)propyl)carbamate (49A). A solution of 2,4-dichloropyrido[3,2-d]pyrimidine (100 mg, 0.5 mmol) in THF (2 mL), was treated with (S)-tert-butyl (2-aminopropyl)carbamate hydrochloride butan-1-amine (CAS #959833-70-6, Fluorochem Ltd. UK), (0.03 mL, 0.56 mmol) and N,N-diisopropylethylamine (0.25 mL, 1.15 mmol). The mixture was stirred at rt for 30 minutes, 2,4-dimethoxybenzylamine (0.19 ml, 1.25 mmol) and N,N-diisopropylethylamine (0.13 mL, 0.75 mmol) were added, and the mixture was heated to 100° C. After 16 h, the reaction was cooled to rt, diluted with EtOAc, washed with water and brine, dried over Na₂SO₄, filtered, and concentrated in vacuo. The resulting residue was subjected to silica gel chromatography eluting with 0-100% EtOAc in hexanes to provide, after removal of volatiles in vacuo, compound 49A. LCMS (m/z): 469.18 [M+H]⁺.

Synthesis of (S)—N⁴-(1-aminopropan-2-yl)pyrido[3,2-d]pyrimidine-2,4-diamine (49). 49A (50 mg, 0.11 mmol) was dissolved in TFA (3 mL). After 30 minutes, the reaction was diluted with water and methanol. After 60 minutes, the mixture was concentrated in vacuo. The residue was then dissolved in methanol and filtered to provide, after removal of volatiles in vacuo, compound 49 as its TFA salt. ¹H NMR (400 MHz, Methanol-d₄) δ 8.67 (ddd, J=9.0, 4.2, 1.6 Hz, 1H), 7.85-7.68 (m, 2H), 4.82 (m, 1H), 3.34 (d, 2H), 1.39 (d, 3H). ¹⁹F NMR (377 MHz, Methanol-d4) δ −77.8. LCMS (m/z): 219.03 [M+H]⁺; t_{R}=0.29 min. (LC/MS HPLC method B).

### Example 50

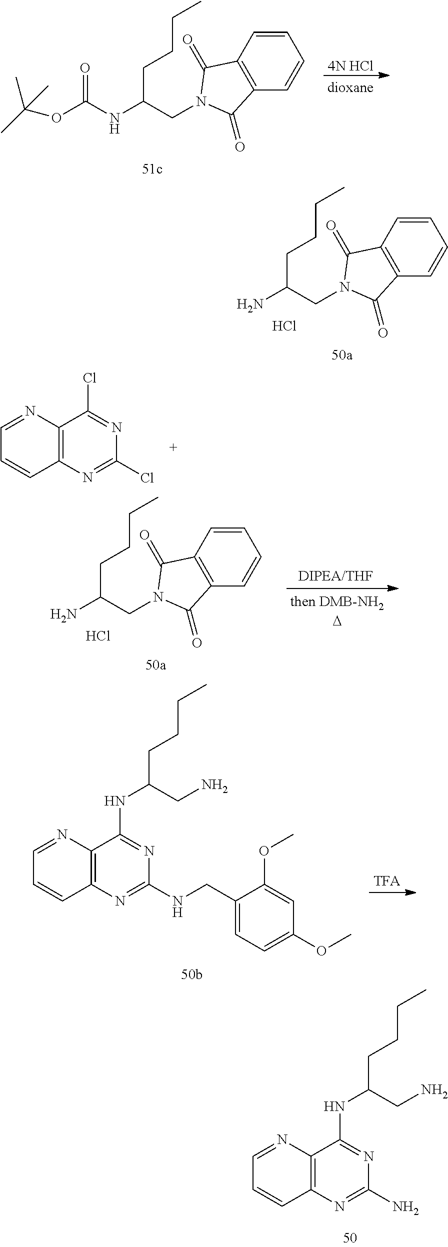

Synthesis of (R)-2-(2-aminohexyl)isoindoline-1,3-dione hydrochloride (50a). To phthalimide 51c (180 mg, 0.53 mmol) was added 4N HCl in dioxane (20 mL). The reaction was stirred at rt for 6 h and then the volatiles were removed in vacuo to provide crude 50a which was carried forward directly into the next step without further purification. LCMS (m/z): 246.93 [M+H]⁺.

Synthesis of (R)-methyl 2-((2-((2,4-dimethoxybenzyl)amino)pyrido[3,2-d]pyrimidin-4-yl)amino)hexanoate (50b). A solution of 2,4-dichloropyrido[3,2-d]pyrimidine (100 mg, 0.5 mmol) in THF (2 mL) was treated with 50a, (150 mg, 0.53 mmol) and N,N-diisopropylethylamine (0.25 mL, 1.15 mmol). The mixture was stirred at rt for 30 minutes, and 2,4-dimethoxybenzylamine (0.38 mL, 2.5 mmol) and N,N-diisopropylethylamine (0.13 mL, 0.75 mmol) were added and the mixture was heated to 125° C. After 24 h, the reaction was cooled to rt, diluted with EtOAc (50 mL), washed with water (25 mL), brine (25 mL), dried over Na₂SO₄, filtered and concentrated in vacuo. The resulting residue was subjected to silica gel chromatography eluting with 0-100% EtOAc in hexanes to give, after removal of volatiles in vacuo, compound 50b.

Synthesis of (R)—N⁴-(1-aminohexan-2-yl)pyrido[3,2-d]pyrimidine-2,4-diamine (50). 50b (15 mg, 0.04 mmol) was dissolved in TFA (3 mL). After 60 minutes the mixture was concentrated to a residue in vacuo followed by co-evaporation with MeOH, to provide the title compound 50 as its bis-TFA salt. ¹H NMR (400 MHz, MeOH-d₄) δ 8.68 (m, 1H), 7.81-7.83 (m, 2H), 4.89 (m, 1H), 3.91 (m, 2H), 3.61 (m, 1H) 1.92-1.79 (m, 2H), 1.55-1.48 (m, 4H), 0.98 (t, J=7.4 Hz, 3H). ¹⁹F NMR (377 MHz, MeOH-d4) δ −77.9. LCMS (m/z): 261.14 [M+H]⁺; t_{R}=0.30 min.

### Example 51

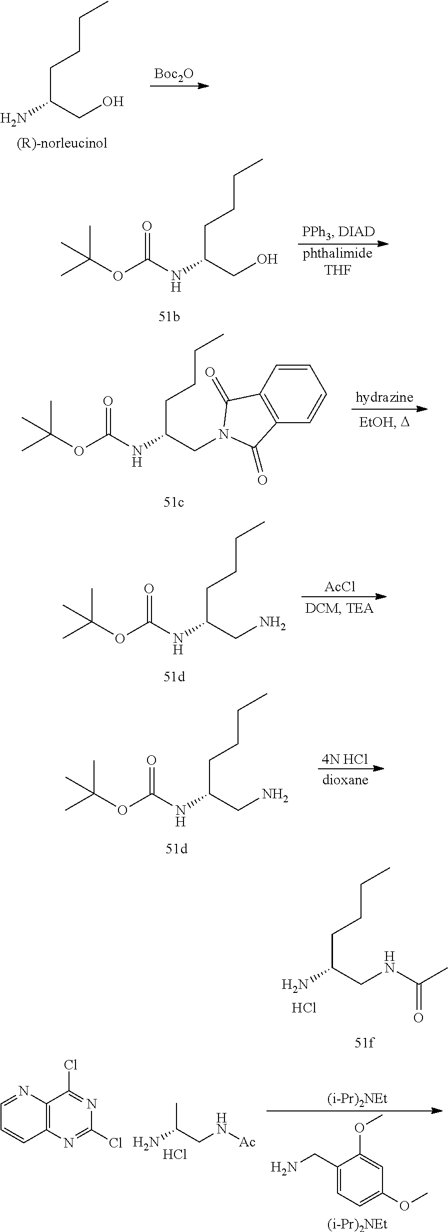

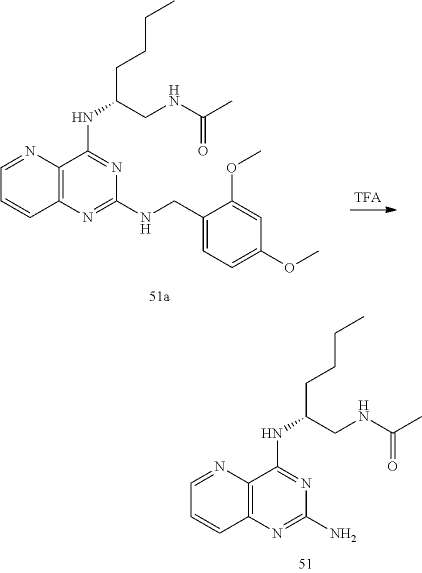 [SMILES: CCCC[C@H](CNC(C)=O)Nc1nc(NCc2ccc(OC)cc2OC)nc2cccnc12 ; CCCC[C@H](CNC(C)=O)Nc1nc(N)nc2cccnc12]

(R)-norleucinol (0.5 g, 4.3 mmol) was treated with Boc₂O (1.2 equiv, 5.2 mmol) and excess N,N-diisopropylethylamine in DCM (20 mL). The reaction mixture was stirred for 3 h and then filtered through a silica gel plug. Removal of the volatiles provided 51b as a crude residue that was used without further purification. LCMS (m/z): 218.23 [M+H]⁺.

Compound 51b (0.7 g, 3.22 mmol) was reacted with PPh₃ (1.1 g, 3.9 mmol), phthalimide (573 mg, 3.9 mmol), and DIAD (810 mg, 4.0 mmol) in THF (30 mL). The mixture was stirred for 3 h, and then partitioned between EtOAc (200 mL) and water (200 mL). The organic layer was separated, washed with brine (100 mL), dried over Na₂SO₄, filtered and concentrated in vacuo. The residue was subjected to silica gel chromatography eluting with 0-100% EtOAc in hexanes to provide 51c. LCMS (m/z): 347.24 [M+H]⁺.

Imide 51c (300 mg, 0.87 mmol) was treated with excess hydrazine hydrate (0.2 mL, 6.25 mmol) in EtOH (30 mL) and refluxed for 16 h. The mixture was concentrated in vacuo to provide intermediate 51d as a crude residue that was carried forward directly. Intermediate 51d (0.87 mmol) was dissolved in DCM (10 mL) and treated with AcCl (0.1 mL, 1.2 mmol), followed by TEA (0.26 mL, 1.8 mmol). The mixture was stirred for 3 h, and then the reaction was diluted with DCM (50 mL). The mixture was then washed with water (50 mL), brine (50 mL), dried over Na₂SO₄, filtered and then concentrated under reduced pressure to provide 51e. LCMS (m/z): 259.21 [M+H]⁺.

Intermediate 51e (0.3 g) was treated with 4N HCl in dioxanes (20 mL) and stirred for 4 h at rt. The volatiles were removed in vacuo to provide the hydrochloride 51f which was used without further purification. LCMS (m/z): 159.45 [M+H]⁺.

Synthesis of (R)—N-(2-((2-((2,4-dimethoxybenzyl)amino)pyrido[3,2-d]pyrimidin-4-yl)amino)hexyl)acetamide (51a). A solution of 2,4-dichloropyrido[3,2-d]pyrimidine (100 mg, 0.5 mmol) in THF (2 mL was treated with 51f, (200 mg, 0.53 mmol) and N,N-diisopropylethylamine (0.25 mL, 1.15 mmol). After the mixture was stirred for 30 minutes, 2,4-dimethoxybenzylamine (0.38 mL, 2.5 mmol) and N,N-diisopropylethylamine (0.13 mL, 0.75 mmol) were added, and the mixture was heated to 115° C. After heating for 16 h, the reaction was cooled to rt, diluted with EtOAc (100 mL), washed with water (100 mL), brine (100 mL), dried over Na₂SO₄, filtered and concentrated in vacuo. The resulting residue was subjected to silica gel flash chromatography eluting with 0-100% EtOAc in hexanes to provide 51a. LCMS (m/z): 453.33 [M+H]⁺.

Synthesis of (R)—N-(2-((2-aminopyrido[3,2-d]pyrimidin-4-yl)amino)hexyl)acetamide (51). 51a (60 mg, 0.133 mmol) was dissolved in TFA (3 mL). After 60 minutes, the mixture was concentrated in vacuo. The residue was taken up in MeOH, filtered and concentrated in vacuo, to give the title compound 51 as its TFA salt. ¹H NMR (400 MHz, MeOH-d₄) 8.65 (dd, J=4.3, 1.5 Hz, 1H), 7.86-7.73 (m, 2H), 4.68-4.55 (m, 4H), 3.59 (dd, J=13.9, 4.3 Hz, 4H), 3.34-3.23 (m, 3H), 1.88 (s, 3H), 1.78-1.67 (m, 2H), 1.39 (ddd, J=7.7, 5.1, 2.4 Hz, 4H), 0.91 (ddt, J=8.3, 4.7, 3.0 Hz, 3H). ¹⁹F NMR (377 MHz, MeOH-d4) δ −77.7. LCMS (m/z): 303.15 [M+H]⁺; t_{R}=0.68 min. (LC/MS HPLC method B).

### Example 52

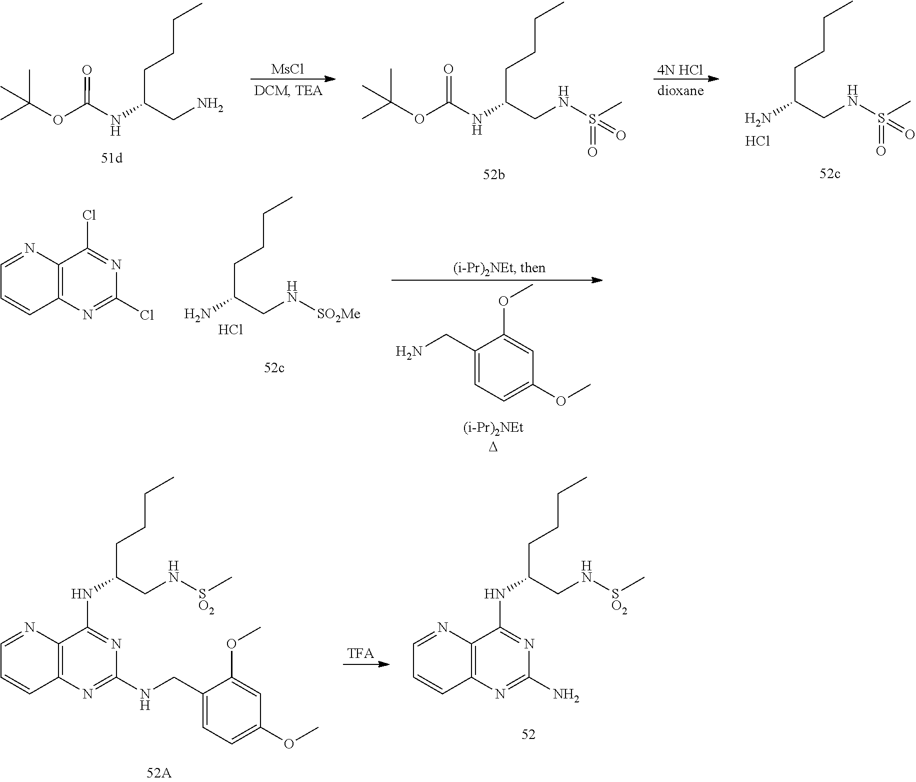

N-Boc-protected intermediate 51d (188 mg, 0.87 mmol) was dissolved in DCM (10 mL) and treated with methanesulfonyl chloride (0.78 μL, 114 mg, 1 mmol) and TEA (0.26 mL, 1.8 mmol). After 3 h, EtOAc (100 mL) was added and the resulting mixture washed with water (100 mL), brine (100 mL), dried over Na2SO4, filtered and concentrated in vacuo to provide 52b. LCMS (m/z): 295.24 [M+H]⁺.

Following the synthesis of 51f from 51e, intermediate 52b (0.87 mmol) was converted to the crude hydrochloride salt 52c which was then carried forward without purification.

Synthesis of (R)—N-(2-((2-((2,4-dimethoxybenzyl)amino)pyrido[3,2-d]pyrimidin-4-yl)amino)hexyl)methanesulfonamide (52A). A solution of 2,4-dichloropyrido[3,2-d]pyrimidine (50 mg, 0.25 mmol) in THF (2 mL) was treated with crude 52c, (85 mg, 0.43 mmol) and N,N-diisopropylethylamine (0.25 mL, 1.15 mmol). The mixture was stirred at rt for 30 minutes, 2,4-dimethoxybenzylamine (0.19 mL, 1.25 mmol) and N,N-diisopropylethylamine (0.13 mL, 0.75 mmol) were added, and the mixture was heated to 115° C. After 16 h, the reaction was cooled to rt, diluted with EtOAc (100 mL), washed with de-ionised water (100 mL), brine (100 mL), dried over Na₂SO₄, filtered and concentrated in vacuo. The residue was subjected to silica gel chromatography eluting with 0-100% EtOAc in hexanes to provide 52A. LCMS (m/z): 489.25 [M+H]⁺.

Synthesis of (R)—N-(2-((2-aminopyrido[3,2-d]pyrimidin-4-yl)amino)hexyl)methanesulfonamide (52). 52A (30 mg, 0.06 mmol) was dissolved in TFA (3 mL). After 60 minutes, the mixture was concentrated in vacuo. The residue was then diluted with MeOH, filtered, and concentrated in vacuo to afford the title product 52 as its TFA salt. ¹H NMR (400 MHz, MeOH-d₄) δ 8.65 (dd, J=4.4, 1.4 Hz, 1H), 7.84 (dd, J=8.5, 1.4 Hz, 1H), 7.76 (dd, J=8.5, 4.4 Hz, 1H), 4.58 (t, J=6.1 Hz, 1H), 3.52-3.26 (m, 2H), 2.93 (s, 3H), 1.75 (dd, J=9.6, 4.0 Hz, 2H), 1.39 (td, J=8.5, 7.6, 3.5 Hz, 4H), 0.91 (m, 3H). ¹⁹F NMR (377 MHz, MeOH-d4) δ −77.7. LCMS (m/z): 339.21 [M+H]⁺; t_{R}=0.83 min. (LC/MS HPLC method B).

### Example 53

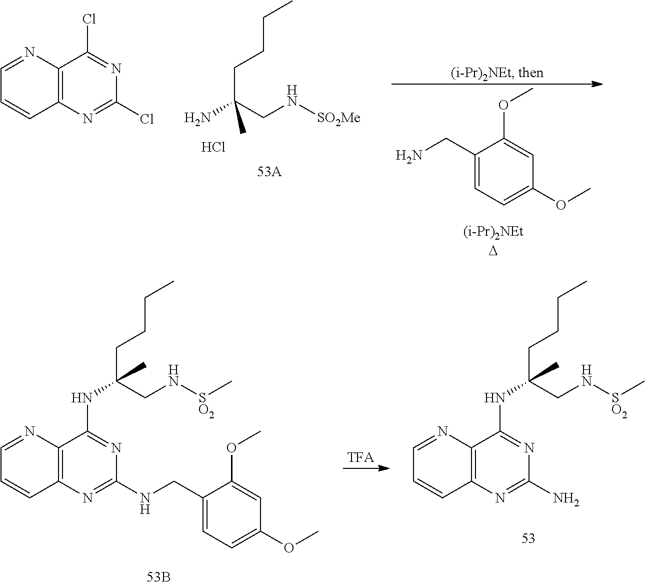

Compound 61C (0.22 g, 0.69 mmol) was mesylated following the procedure for the formation of 61D but instead replacing acetyl chloride with methanesulfonyl chloride (0.06 mL, 0.8 mmol) to give a quantitative yield of the corresponding mesylated intermediate. The resulting sulfonamide was then subjected to Pd/C hydrogenation followed by N—BOC removal, as described in the preparation of 61E from 61D to give the crude product 53A as its hydrochloride salt. LCMS (m/z): 209.1 [M+H]⁺.

Synthesis of (R)—N-(2-((2-((2,4-dimethoxybenzyl)amino)pyrido[3,2-d]pyrimidin-4-yl)amino)-2-methylhexyl)methanesulfonamide (53B). A solution of 2,4-dichloropyrido[3,2-d]pyrimidine (100 mg, 0.5 mmol) in THF (4 mL) was treated with crude 53A (0.69 mmol), and N,N-diisopropylethylamine (0.5 mL, 2.3 mmol). After heating at 75° C. for 4 h, 2,4-dimethoxybenzylamine (0.4 mL, 2.5 mmol) and additional N,N-diisopropylethylamine (0.26 mL, 1.5 mmol) were added and the mixture was heated to 115° C. After 16 h, the reaction was cooled to rt, diluted with EtOAc (100 mL), washed with de-ionised water (100 mL), brine (100 mL), dried over Na₂SO₄, filtered and concentrated under reduced pressure. The residue was subjected to silica gel chromatography eluting with 0-100% EtOAc to give 53B. LCMS (m/z): 503.28 [M+H]⁺.

Synthesis of (R)—N-(2-((2-aminopyrido[3,2-d]pyrimidin-4-yl)amino)-2-methylhexyl)methanesulfonamide (53). 53B (75 mg, 0.15 mmol) was dissolved in TFA (3 mL). After 60 minutes, the mixture was concentrated in vacuo. The residue was dissolved in MeOH, filtered and volatiles removed in vacuo to afford the title product 53, as its TFA salt. ¹H NMR (400 MHz, MeOH-d₄) δ 8.63 (dd, J=4.3, 1.4 Hz, 1H), 7.79 (dd, J=8.4, 1.5 Hz, 1H), 7.73 (dd, J=8.4, 4.3 Hz, 1H), 3.78 (m, 2H), 2.93 (s, 3H), 2.25 (m, 1H), 1.82 (dd, J=9.6, 4.0 Hz, 2H), 1.56 (s, 3H), 1.37 (td, J=8.4, 7.5, 3.4 Hz, 4H), 0.93 (m, 3H). ¹⁹F NMR (377 MHz, MeOH-d4) δ −77.6. LCMS (m/z): 353.18 [M+H]⁺; t_{R}=0.83 min. (LC/MS HPLC method B).

### Example 54

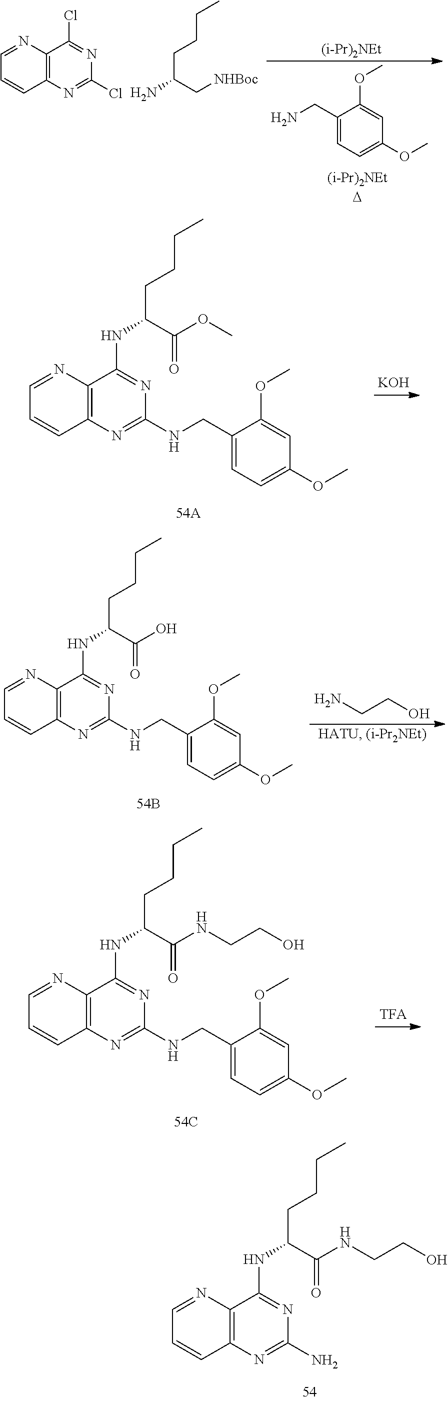

Synthesis of (R)-methyl 2-((2-((2,4-dimethoxybenzyl)amino)pyrido[3,2-d]pyrimidin-4-yl)amino)hexanoate (54A). To a solution of 2,4-dichloropyrido[3,2-d]pyrimidine (CAS #39551-54-7, supplied by Astatech, Inc.) (500 mg, 2.5 mmol) in THF (10 mL) was added D-norleucine methyl ester hydrochloride (454 mg, 2.5 mmol) and N,N-diisopropylethylamine (1.3 mL, 7.5 mmol). After stirring at rt for 30 minutes, 2,4-dimethoxybenzylamine (1.9 mL, 12.5 mmol) and N,N-diisopropylethylamine (1.3 mL, 7.5 mmol) were added and the mixture was heated to 100° C. After 16 h, the reaction was cooled to rt, diluted with EtOAc (100 mL), washed with water (100 mL), brine (100 mL), dried over Na₂SO₄, filtered and concentrated in vacuo. The residue was subjected to silica gel chromatography eluting with hexanes-EtOAc to provide 54A. ¹H NMR (400 MHz, Chloroform-d) δ 8.33 (dd, J=4.2, 1.5 Hz, 1H), 7.68 (d, J=7.6 Hz, 1H), 7.43 (dd, J=8.5, 4.2 Hz, 1H), 7.28 (s, 1H), 6.46 (d, J=2.3 Hz, 1H), 6.41 (dd, J=8.2, 2.4 Hz, 1H), 4.88 (q, J=7.3 Hz, 1H), 4.59 (d, J=6.0 Hz, 2H), 3.85 (s, 3H), 3.79 (s, 3H), 3.75 (s, 3H), 2.04-1.95 (m, 1H), 1.88 (dq, J=14.8, 7.6 Hz, 1H), 1.40 (dddd, J=26.8, 15.8, 6.9, 2.6 Hz, 5H), 0.91 (t, J=7.1 Hz, 3H). LCMS (m/z): 440.49 [M+H]⁺; t_{R}=0.77 min. on LC/MS Method A.

Synthesis of (R)-2-((2-((2,4-dimethoxybenzyl)amino)pyrido[3,2-d]pyrimidin-4-yl)amino)hexanoic acid (54B). To a solution of 54A (750.7 mg, 1.71 mL) in THF (3.6 mL) and MeOH (3.6 mL) was added 1N KOH_{(aq)} (3.6 mL). After 4 h, the reaction was was neutralized to pH 7 using 1M HCl_{(aq)}. Concentration of the mixture in vacuo afforded the crude product 54B. ¹H NMR (400 MHz, DMSO-d₆) δ 8.34 (d, J=4.1 Hz, 1H), 7.77 (s, 1H), 7.61 (d, J=6.5 Hz, 1H), 7.53 (dd, J=8.5, 4.2 Hz, 1H), 7.10 (s, 1H), 6.53 (d, J=2.3 Hz, 1H), 6.42 (dd, J=7.9, 2.0 Hz, 1H), 4.65 (s, 1H), 4.44 (s, 2H), 3.81 (s, 3H), 3.71 (s, 3H), 1.90 (s, 2H), 1.30 (s, 4H), 0.84 (s, 3H). LCMS (m/z): 426.16 [M+H]⁺; t_{R}=0.67 min. on LC/MS Method A.

Synthesis of (R)-2-((2-((2,4-dimethoxybenzyl)amino)pyrido[3,2-d]pyrimidin-4-yl)amino)-N-(2-hydroxyethyl)hexanamide (54C). To a solution of crude 54B (50 mg, 0.12 mmol), N,N-diisopropylethylamine (0.15 mL, 0.86 mmol), and 2-aminoethanol (0.05 mL, 0.59 mmol) in NMP (12 mL) was added HATU (96 mg, 0.25 mmol). After 16 h the mixture was subjected to preparative HPLC (Synergi 4 u Polar-RP 80 A, Axia; 10% aq. acetonitrile-70% aq. acetonitrile with 0.1% TFA, over 20 min. gradient) to afford 54C as its TFA salt. LCMS (m/z): 469.23 [M+H]⁺; t_{R}=0.70 min. on LC/MS Method A.

Synthesis of (R)-2-((2-aminopyrido[3,2-d]pyrimidin-4-yl)amino)-N-(2-hydroxyethyl)hexanamide (54). To 54C (10 mg, 0.02 mmol) was added TFA (3 mL). After 4 h, MeOH (2 mL) and water (2 mL) were added to the mixture. After 16 h, the mixture was concentrated in vacuo and then co-evaporated with MeOH three times. The residue was subjected to preparative HPLC (Synergi 4 u Polar-RP 80 A, Axia; 10% aq. acetonitrile-60% aq. acetonitrile with 0.1% TFA, over 20 min. gradient) to give 54 as a TFA salt. ¹H NMR (400 MHz, MeOH-d₄) δ 8.68 (dd, J=4.4, 1.5 Hz, 1H), 7.86 (dd, J=8.5, 1.5 Hz, 1H), 7.80 (dd, J=8.5, 4.4 Hz, 1H), 4.81 (dd, J=8.2, 5.7 Hz, 1H), 3.66-3.56 (m, 2H), 3.43-3.32 (m, 2H), 2.12-1.90 (m, 2H), 1.49-1.36 (m, 4H), 0.98-0.89 (m, 3H). ¹⁹F NMR (377 MHz, MeOH-d₄) δ −77.83. LCMS (m/z): 319.23 [M+H]⁺; t_{R}=0.49 min. on LC/MS Method A.

### Example 55

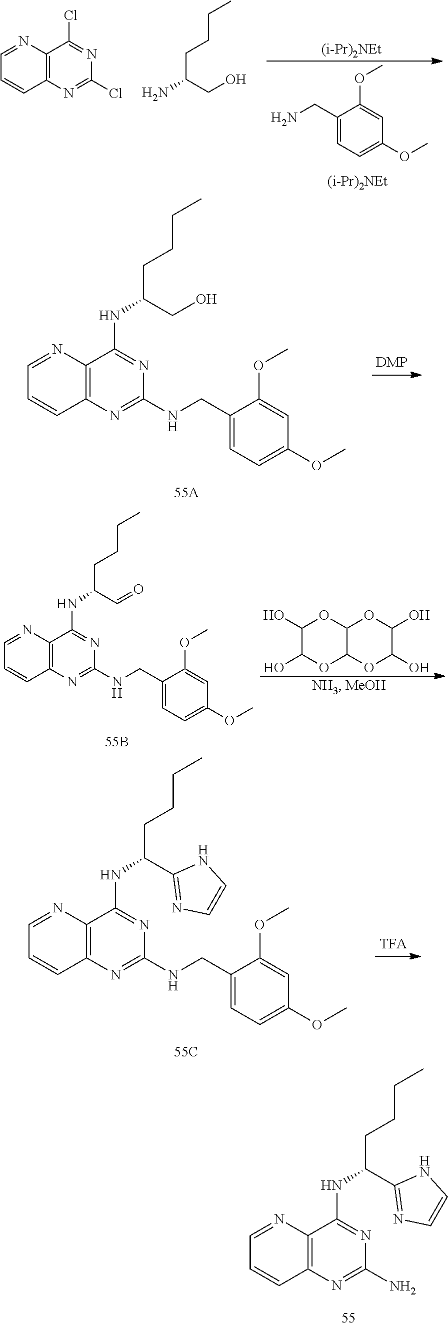

Synthesis of (R)-2-((2-((2,4-dimethoxybenzyl)amino)pyrido[3,2-d]pyrimidin-4-yl)amino)hexan-1-ol (55A). To a solution of 2,4-dichloropyrido[3,2-d]pyrimidine (500 mg, 2.5 mmol) in THF (15 mL) was added (R)-norleucinol (293 mg, 2.5 mmol) and N,N-diisopropylethylamine (1.3 mL, 7.5 mmol). After stirring at rt for 30 minutes, 2,4-dimethoxybenzylamine (1.9 mL, 12.5 mmol) and N,N-diisopropylethylamine (1.3 mL, 7.5 mmol) were added and the mixture was heated to 100° C. After 16 h, the reaction was cooled to rt, diluted with EtOAc (100 mL), washed with water (100 mL), brine (100 mL), dried over Na₂SO₄, filtered and concentrated in vacuo. The residue was subjected to silica gel chromatography eluting with hexanes-EtOAc to give 55A. ¹H NMR (400 MHz, Chloroform-d) δ 8.32 (s, 1H), 7.74 (s, 1H), 7.46 (s, 1H), 6.49-6.37 (m, 3H), 4.60 (d, J=5.9 Hz, 3H), 3.86 (s, 5H), 3.79 (s, 5H), 1.55 (s, 2H), 1.45-1.33 (m, 6H), 0.91 (t, J=7.0 Hz, 4H). LCMS (m/z): 412.20 [M+H]⁺; t_{R}=0.89 min. on LC/MS Method A.

Synthesis of (R)-2-((2-((2,4-dimethoxybenzyl)amino)pyrido[3,2-d]pyrimidin-4-yl)amino)hexanal (55B). To a solution of 55A (100 mg, 0.24 mmol) in DCM (5 mL) at 0° C. was added Dess-Martin periodinane (248 mg, 0.58 mmol). The reaction was warmed to rt and stirred for 24 h. The reaction was diluted with DCM (5 mL) and then quenched with a mixture of sat. Na₂S₂O_{3(aq)} (5 mL) and sat. NaHCO_{3(aq)} (5 mL). The organic layer was separated and the aqueous layer was extracted with DCM (2×10 mL). The combined organics were washed with brine (100 mL), dried over Na₂SO₄, filtered and concentrated in vacuo. The residue was subjected to silica gel chromatography eluting with hexanes-EtOAc to give 55B. LCMS (m/z): 410.19 [M+H]⁺; t_{R}=0.97 min. on LC/MS Method A.

Synthesis of (R)—N⁴-(1-(1H-imidazol-2-yl)pentyl)-N²-(2,4-dimethoxybenzyl)pyrido[3,2-d]pyrimidine-2,4-diamine (55C). To a solution of 55B (50 mg, 0.12 mmol) in MeOH (2 mL) was added glyoxal trimer dihydrate (12 mg, 0.06 mg) and ammonia in MeOH (2M, 0.28 mL, 0.55 mmol). After 24 h, additional glyoxal trimer dihydrate (12 mg, 0.06 mg) and ammonia in MeOH (2M, 0.28 mL, 0.55 mmol) were added. After 18 h, the mixture was concentrated in vacuo. The residue was diluted with water (10 mL) and extracted with EtOAc (4×10 mL). The combined organics were dried over Na₂SO₄, filtered and concentrated in vacuo to afford the crude 55C. LCMS (m/z): 448.15 [M+H]⁺; t_{R}=0.62 min. on LC/MS Method A.

Synthesis of (R)—N⁴-(1-(1H-imidazol-2-yl)pentyl)pyrido[3,2-d]pyrimidine-2,4-diamine (55). To 55C (50 mg, 0.11 mmol) was added TFA (2 mL). After 90 minutes, MeOH (2 mL) and water (2 mL) were added to the mixture. After 16 h, the mixture was concentrated in vacuo and co-evaporated with MeOH (×3). The residue was subjected to preparative HPLC (Synergi 4 u Polar-RP 80 A, Axia; 10% aq. acetonitrile-60% aq. acetonitrile with 0.1% TFA, over 20 min. gradient) to give 55 as a TFA salt ¹H NMR (400 MHz, MeOH-d₄) δ 8.70 (dd, J=4.4, 1.4 Hz, 1H), 7.93 (dd, J=8.5, 1.4 Hz, 1H), 7.83 (dd, J=8.5, 4.4 Hz, 1H), 7.52 (s, 2H), 5.92-5.71 (m, 1H), 2.30 (td, J=9.3, 8.7, 4.3 Hz, 2H), 1.64-1.34 (m, 4H), 0.95 (t, J=7.0 Hz, 3H). ¹⁹F NMR (377 MHz, MeOH-d₄) δ −77.73. LCMS (m/z): 298.05 [M+H]⁺; t_{R}=0.46 min. on LC/MS Method A.

### Example 56

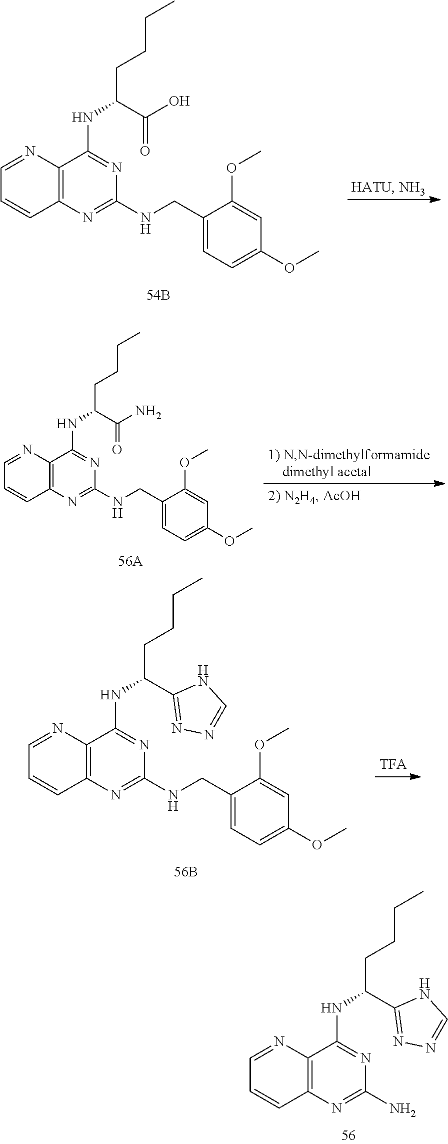

Synthesis of (R)-2-((2-((2,4-dimethoxybenzyl)amino)pyrido[3,2-d]pyrimidin-4-yl)amino)hexanamide (56A). To a solution of 54B (50 mg, 0.12 mmol), N,N-diisopropylethylamine (0.1 mL, 0.57 mmol), and ammonia in dioxane (0.5 M, 1.2 mL, 0.59 mmol) in NMP (6 mL) was added HATU (174 mg, 0.46 mmol). After 4 h the mixture was subjected to preparative HPLC (Synergi 4 u Polar-RP 80 A, Axia; 10% aq. acetonitrile-70% aq. acetonitrile with 0.1% TFA, over 20 min. gradient) to afford 56A as a TFA salt. LCMS (m/z): 425.18 [M+H]⁺; t_{R}=0.69 min. on LC/MS Method A.

Synthesis of (R)—N⁴-(1-(4H-1,2,4-triazol-3-yl)pentyl)-N²-(2,4-dimethoxybenzyl)pyrido[3,2-d]pyrimidine-2,4-diamine (56B). A mixture of 56A (70 mg, 0.17 mmol) and N,N-dimethylformamide dimethyl acetal (2 mL, 16 mmol) was heated to 120° C. After 2 h, the mixture was cooled to rt and concentrated in vacuo. The crude residue was dissolved in AcOH (2 mL) and treated with hydrazine monohydrate (0.02 mL, 0.42 mmol). The mixture was heated to 90° C. for 24 h. The mixture was concentrated in vacuo to afford the crude 56B which was used without further purification. LCMS (m/z): 449.23 [M+H]⁺; t_{R}=0.83 min. on LC/MS Method A.

Synthesis of (R)—N⁴-(1-(4H-1,2,4-triazol-3-yl)pentyl)pyrido[3,2-d]pyrimidine-2,4-diamine (56). To crude 56B was added TFA (3 mL). After 60 minutes, the mixture was concentrated in vacuo and the residue was diluted with MeOH (3.5 mL) and water (3.5 mL). After 90 min., the mixture was concentrated and then subjected to preparative HPLC (Synergi 4 u Polar-RP 80 A, Axia; 10% aq. acetonitrile-60% aq. acetonitrile with 0.1% TFA, over 20 min. gradient) to afford 56 as a TFA salt. ¹H NMR (400 MHz, MeOH-d₄) δ 8.67 (dd, J=4.4, 1.4 Hz, 1H), 8.47 (s, 1H), 7.86 (dd, J=8.5, 1.4 Hz, 1H), 7.79 (dd, J=8.5, 4.4 Hz, 1H), 5.72 (dd, J=8.4, 6.3 Hz, 1H), 2.30-2.09 (m, 2H), 1.49-1.34 (m, 4H), 0.96-0.89 (m, 3H). ¹⁹F NMR (377 MHz, MeOH-d₄) δ −77.98. LCMS (m/z): 299.15 [M+H]⁺; t_{R}=0.62 min. on LC/MS Method A.

### Example 57

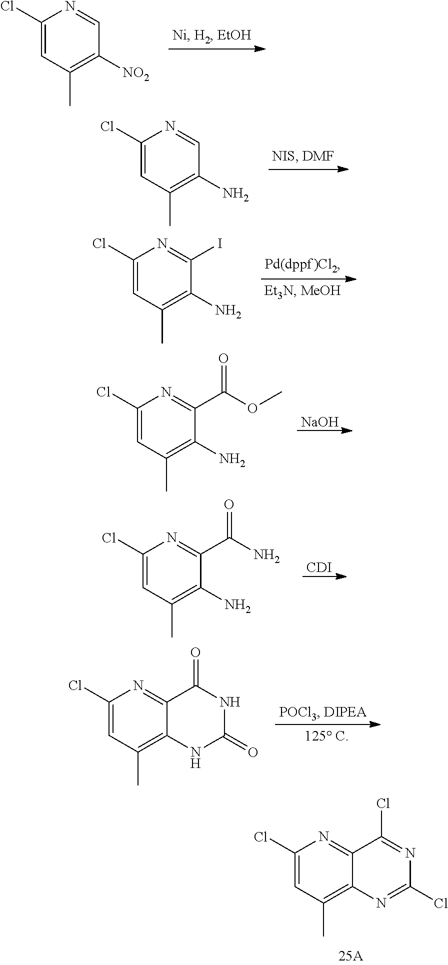

2-Chloro-4-methyl-5-nitropyridine (10.0 g, 57.8 mmol) was dissolved in EtOH (100 mL) and Raney nickel (3 g) was added. The reaction mixture was stirred under H₂ overnight. The mixture was filtered, concentrated under vacuum, and washed with petroleum ether/EtOAc=5:1 (50 mL) to give crude 6-chloro-4-methylpyridin-3-amine.

6-Chloro-4-methylpyridin-3-amine (22.0 g, 154.9 mmol) was dissolved in DMF (150 mL) and treated with NIS (41.8 g, 185.9 mmol). The reaction mixture was stirred at rt overnight, then water (200 mL) was added, and the mixture was extracted with EtOAc (3×200 mL). The combined organics were concentrated in vacuo and the residue was subjected to silica gel flash chromatography eluting with Et₂O-EtOAc to give 6-chloro-2-iodo-4-methylpyridin-3-amine. ¹H NMR (DMSO-d₆, 400 MHz): δ 7.11 (s, 1H), 5.23 (s, 2H), 2.15 (s, 3H) ppm.

To a solution of 6-chloro-2-iodo-4-methylpyridin-3-amine (30.0 g, 111.7 mmol) in MeOH (200 mL) was added Pd(dppf)Cl₂ (4.09 g, 5.5 mmol), Et₃N (45.1 g, 447 mmol) and the reaction mixture was stirred at rt overnight. The residue was subjected to silica gel chromatography eluting with Et₂O-EtOAc to give 6-chloro-2-iodo-4-methylpyridin-3-amine. ¹H NMR (DMSO-d₆, 400 MHz): δ 7.33 (d, J=0.8, 1H), 6.74 (s, 2H), 3.82 (s, 3H), 3.18 (d, J=0.4, 3H) ppm.

To a solution of 6-chloro-2-iodo-4-methylpyridin-3-amine (18.8 g, 94 mmol) in NH₄OH (180 mL) was added MeOH (10 mL) and the reaction mixture was stirred at rt overnight. The mixture was filtered and the collected solid washed with petroleum ether/EtOAc (5:1, 50 mL) to afford 3-amino-6-chloro-4-methylpicolinamide. ¹H NMR (DMSO-d₆, 400 MHz): δ 7.76 (s, 1H), 7.43 (s, 1H), 7.27 (s, 1H), 6.92 (s, 2H), 2.15 (s, 3H) ppm.

A solution of 3-amino-6-chloro-4-methylpicolinamide (10 g, 54.1 mmol) and CDI (8.02 g; 27.02 mmol) in 1,4-dioxane (200 mL) was stirred at 110° C. for 30 minutes. The mixture was filtered and the collected solids were washed with EtOAc (30 mL). The organics were concentrated in vacuo to give crude 6-chloro-8-methylpyrido[3,2-d]pyrimidine-2,4(1H,3H)-dione. ¹H NMR (CDCl₃, 400 MHz) δ 7.70 (d, J=1.2 Hz, 1H), 2.76 (d, J=0.8 Hz, 3H) ppm.

Synthesis of 2,4,6-trichloro-8-methylpyrido[3,2-d]pyrimidine (25A). A solution of 6-chloro-8-methylpyrido[3,2-d]pyrimidine-2,4(1H,3H)-dione (32 g, 151.6 mmol) and N,N-diisopropylethylamine (50 mL) in POCl₃ (320 mL) was stirred at 125° C. overnight. The mixture was concentrated in vacuo and the residue was subjected to silica gel flash chromatography eluting with Et₂O-EtOAc to give 25A. ¹H NMR (CDCl₃, 400 MHz) δ 7.70 (d, J=1.2 Hz, 1H), 2.76 (d, J=0.8 Hz, 3H) ppm.

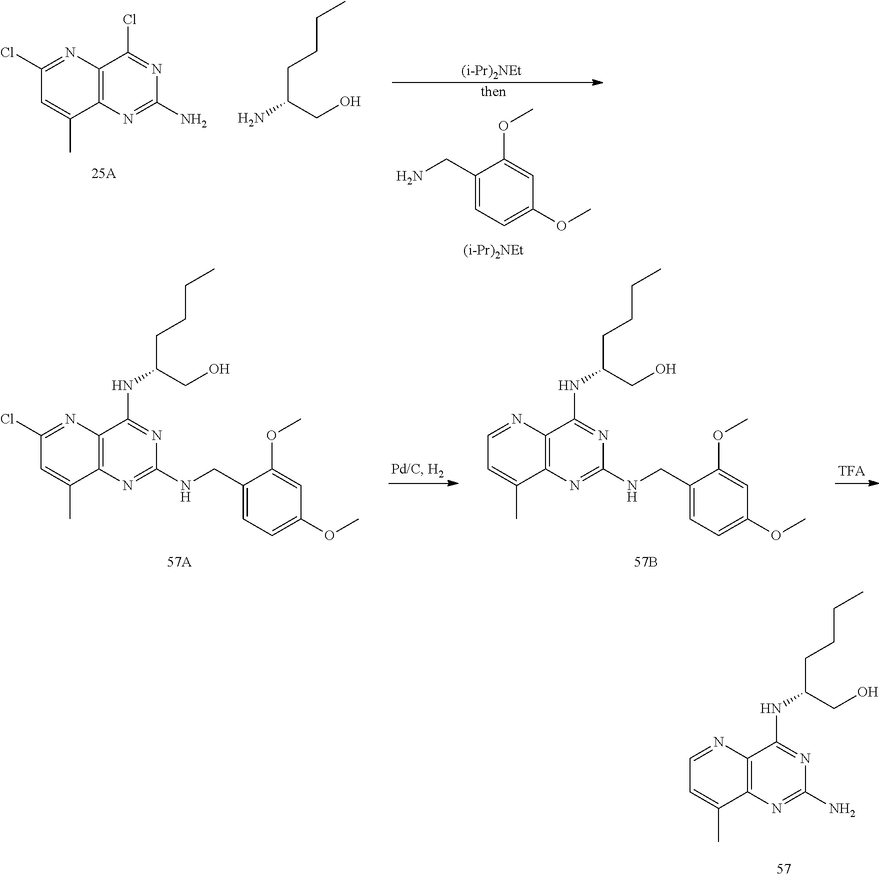

Synthesis of (R)-2-((6-chloro-2-((2,4-dimethoxybenzyl)amino)-8-methylpyrido[3,2-d]pyrimidin-4-yl)amino)hexan-1-ol (57A). To a solution of 25A (50 mg, 0.20 mmol) in THF (15 mL) was added D-norleucinol (24 mg, 0.20 mmol) and N,N-diisopropylethylamine (1.1 mL, 6.0 mmol). After stirring at rt for 30 minutes, 2,4-dimethoxybenzylamine (0.2 mL, 1.1 mmol) and additional N,N-diisopropylethylamine (0.26 mL, 1.5 mmol) was added and the mixture was heated to 100° C. After 16 h, the reaction was cooled to rt, diluted with EtOAc (100 mL), washed with water (100 mL), brine (100 mL), dried over Na₂SO₄, filtered and concentrated in vacuo. The crude residue was subjected to silica gel chromatography eluting with hexanes-EtOAc to provide 57A. ¹H NMR (400 MHz, Chloroform-d) δ 7.30 (d, J=8.2 Hz, 1H), 7.25 (s, 1H), 6.75 (d, J=6.0 Hz, 1H), 6.46 (d, J=2.3 Hz, 1H), 6.41 (dd, J=8.2, 2.4 Hz, 1H), 5.39 (s, 1H), 4.57 (d, J=6.0 Hz, 2H), 3.85 (s, 4H), 3.81 (d, J=3.1 Hz, 1H), 3.79 (s, 4H), 3.68 (q, J=7.7, 7.2 Hz, 1H), 2.51 (s, 3H), 1.72-1.60 (m, 3H), 1.46-1.30 (m, 5H), 0.95-0.86 (m, 4H). LCMS (m/z): 460.25 [M+H]⁺; t_{R}=1.26 min. on LC/MS Method A.

Synthesis of (R)-2-((2-((2,4-dimethoxybenzyl)amino)-8-methylpyrido[3,2-d]pyrimidin-4-yl)amino)hexan-1-ol (57B). A solution of 57A (35 mg, 0.08 mmol) in EtOAc (4 mL) and EtOH (4 mL) was purged with Ar, and then Pd/C (Degussa 10 wt %, 25 mg) was added. The mixture was then purged with H₂ and heated to 70° C. After 1 h, the reaction was cooled, purged with Ar, filtered through Celite, and the Celite rinsed with EtOAc. The organics were concentrated in vacuo and the residue was subjected to silica gel chromatography eluting with EtOAc-MeOH to afford 57B. LCMS (m/z): 426.16 [M+H]⁺; t_{R}=1.18 min. on LC/MS Method A.

Synthesis of (R)-2-((2-amino-8-methylpyrido[3,2-d]pyrimidin-4-yl)amino)hexan-1-ol (57). To 57B (21 mg, 0.05 mmol) was added TFA (3 mL). After 60 minutes, MeOH (5 mL) and water (5 mL) were added to the mixture. After 4 h, the mixture was concentrated in vacuo and co-evaporated with MeOH (×3). The residue was subjected to preparative HPLC (Synergi 4 u Polar-RP 80 A, Axia; 10% aq. acetonitrile-70% aq. acetonitrile with 0.1% TFA, over 20 min. gradient) to provide 57 as a TFA salt. ¹H NMR (400 MHz, MeOH-d₄) δ 8.50 (d, J=4.6 Hz, 1H), 7.63 (dd, J=4.6, 1.0 Hz, 1H), 4.53 (dq, J=8.6, 5.2 Hz, 1H), 3.74 (d, J=5.3 Hz, 2H), 2.53 (d, J=0.8 Hz, 4H), 1.83-1.64 (m, 3H), 1.45-1.33 (m, 5H), 0.97-0.87 (m, 4H). ¹⁹F NMR (377 MHz, MeOH-d₄) δ −77.78. LCMS (m/z): 276.26 [M+H]⁺; t_{R}=0.88 min. on LC/MS Method A.

### Example 58

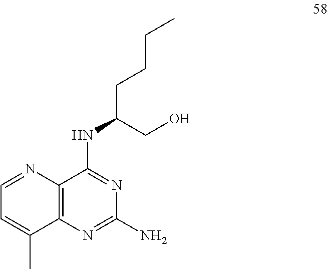 [SMILES: CCCC[C@@H](CO)Nc1nc(N)nc2c(C)ccnc12]

Synthesis of (S)-2-((2-amino-8-methylpyrido[3,2-d]pyrimidin-4-yl)amino)hexan-1-ol (58). 58 was synthesized in a 3 step procedure similar to that described for Example 57, instead replacing D-norleucinol with L-norleucinol (24 mg, 0.204 mmol), affording 58 as a TFA salt. ¹H NMR (400 MHz, MeOH-d₄) δ 8.48 (d, J=4.6 Hz, 1H), 7.60 (dd, J=4.6, 1.0 Hz, 1H), 4.52 (dq, J=8.7, 5.4 Hz, 1H), 3.74 (d, J=5.8 Hz, 2H), 2.52 (d, J=0.8 Hz, 3H), 1.86-1.61 (m, 3H), 1.47-1.32 (m, 5H), 0.95-0.86 (m, 4H). ¹⁹F NMR (377 MHz, MeOH-d₄) δ −77.64. LCMS (m/z): 276.17 [M+H]⁺; t_{R}=0.88 min. on LC/MS Method A.

### Example 59

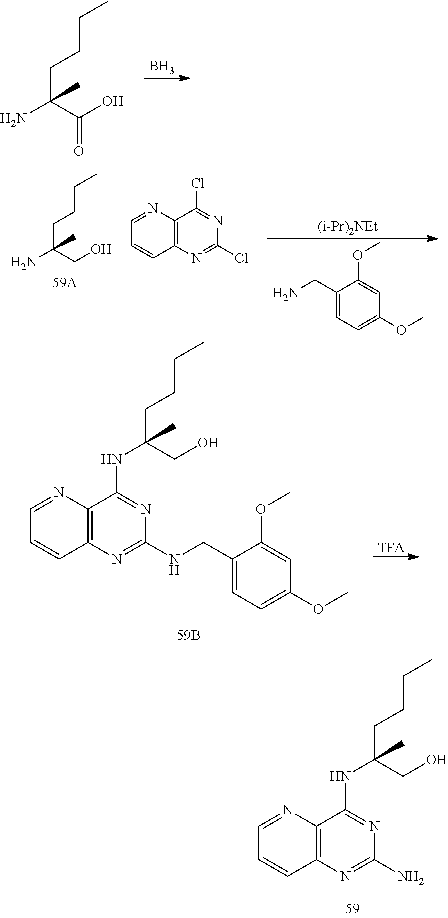

Synthesis of (R)-2-amino-2-methylhexan-1-ol (59A). To (2R)-2-amino-2-methylhexanoic acid hydrochloride (250 mg, 1.4 mmol, supplied by Astatech) in THF (5 mL) was added borane-tetrahydrofuran complex solution in THF (1M, 5.5 mL) dropwise over 5 minutes. After 24 h, the reaction was quenched with MeOH (1 mL) and concentrated in vacuo. The residue was diluted with DCM, filtered, and concentrated in vacuo to afford crude 59A which was carried forward into the next step directly. LCMS (m/z): 131.92 [M+H]⁺; t_{R}=0.58 min. on LC/MS Method A.

Synthesis of (R)-2-((2-((2,4-dimethoxybenzyl)amino)pyrido[3,2-d]pyrimidin-4-yl)amino)-2-methylhexan-1-ol (59B). To a solution of 2,4-dichloropyrido[3,2-d]pyrimidine (50 mg, 0.25 mmol) in THF (10 mL) was added 59A (50 mg, 0.38 mmol) and N,N-diisopropylethylamine (0.13 mL, 0.75 mmol). After stirring at 80° C. for 18 h, 2,4-dimethoxybenzylamine (0.19 mL, 1.25 mmol) was added and the mixture was heated to 100° C. After 18 h, the reaction was cooled to rt, diluted with EtOAc, washed with water and brine, dried over Na₂SO₄, then filtered and concentrated in vacuo. The residue was subjected to silica gel chromatography eluting with hexanes-EtOAc to provide 59B. LCMS (m/z): 426.21 [M+H]⁺; t_{R}=0.91 min. on LC/MS Method A.

Synthesis of (R)-2-((2-aminopyrido[3,2-d]pyrimidin-4-yl)amino)-2-methylhexan-1-ol (59). To 59B was added TFA (3 mL). After 2 h, the reaction mixture was concentrated in vacuo. The residue was subjected to preparative HPLC (Synergi 4 u Polar-RP 80 A, Axia; 10% aq. acetonitrile-70% aq. acetonitrile with 0.1% TFA, over 20 min. gradient) to provide 59 as a TFA salt. ¹H NMR (400 MHz, Methanol-d₄) δ 8.62 (dd, J=4.2, 1.6 Hz, 1H), 7.81 (dd, J=8.5, 1.6 Hz, 1H), 7.77 (dd, J=8.5, 4.2 Hz, 1H), 3.97 (d, J=11.2 Hz, 1H), 3.72 (d, J=11.2 Hz, 1H), 2.18-2.03 (m, 1H), 1.99-1.86 (m, 1H), 1.54 (s, 3H), 1.41-1.30 (m, 4H), 0.92 (t, J=6.9 Hz, 2H). ¹⁹F NMR (377 MHz, MeOH-d₄) δ −77.98. LCMS (m/z): 276.13 [M+H]⁺; t_{R}=0.65 min. on LC/MS Method A.

### Example 60

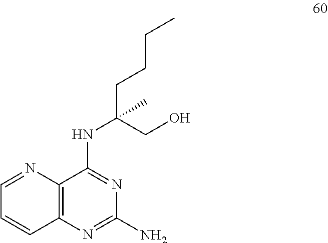 [SMILES: CCCC[C@@](C)(CO)Nc1nc(N)nc2cccnc12]

Synthesis of (S)-2-((2-aminopyrido[3,2-d]pyrimidin-4-yl)amino)-2-methylhexan-1-ol (60). Compound 60 was synthesized in a procedure similar to that reported for 59, replacing (2R)-2-amino-2-methylhexanoic acid hydrochloride with (2S)-2-amino-2-methylhexanoic acid hydrochloride (250 mg, 1.38 mmol, supplied by Astatech, Inc.). Final purification with preparative HPLC (Synergi 4 u Polar-RP 80 A, Axia; 10% aq. acetonitrile-70% aq. acetonitrile with 0.1% TFA, over 20 min. gradient) provided 60 as a TFA salt. ¹H NMR (400 MHz, Methanol-d₄) δ 8.63 (dd, J=4.3, 1.5 Hz, 1H), 7.82 (dd, J=8.5, 1.5 Hz, 1H), 7.77 (dd, J=8.5, 4.3 Hz, 1H), 3.98 (d, J=11.2 Hz, 1H), 3.73 (d, J=11.2 Hz, 1H), 2.19-2.04 (m, 1H), 2.01-1.88 (m, 1H), 1.55 (s, 3H), 1.50-1.29 (m, 4H), 0.93 (t, J=6.9 Hz, 3H). ¹⁹F NMR (377 MHz, MeOH-d₄) δ −77.98. LCMS (m/z): 276.10 [M+H]⁺; t_{R}=0.65 min. on LC/MS Method A.

### Example 61

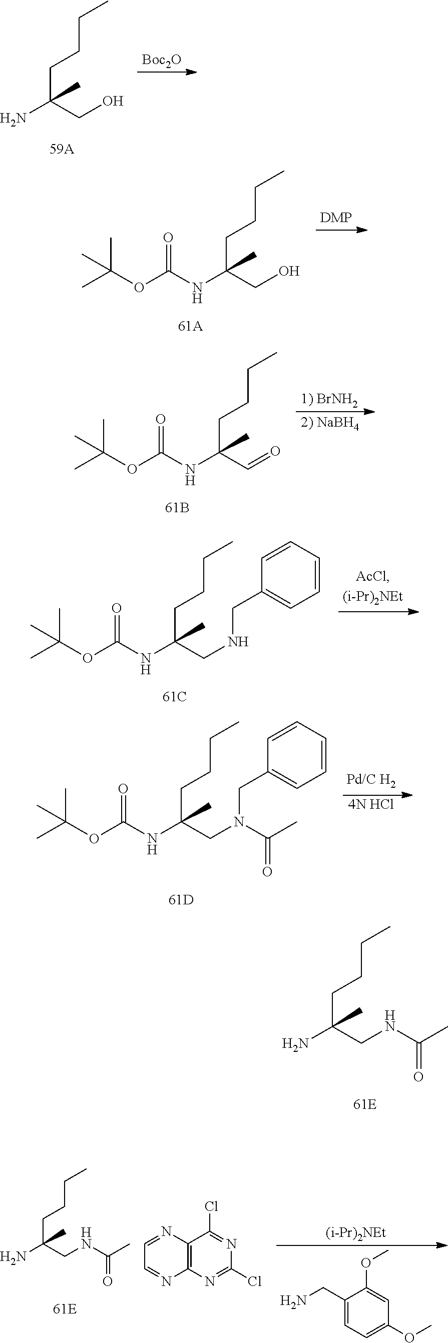

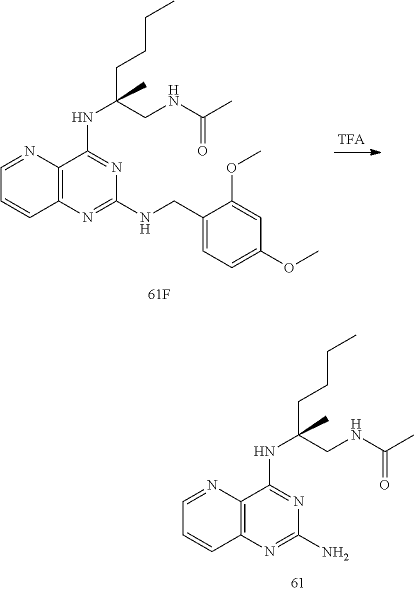 [SMILES: CCCC[C@](C)(CNC(C)=O)Nc1nc(NCc2ccc(OC)cc2OC)nc2cccnc12 ; CCCC[C@](C)(CNC(C)=O)Nc1nc(N)nc2cccnc12]

Synthesis of (R)-tert-butyl (1-hydroxy-2-methylhexan-2-yl)carbamate (61A). To a solution of 59A (1 g, 7.6 mmol) in THF (35 mL) was added sat. NaHCO_{3(aq)} (35 mL) followed by di-tert-butyl dicarbonate (3.33 g, 15.24 mmol). After 24 h, the organic solvents were removed in vacuo. The resulting slurry was diluted with water (50 mL), extracted with EtOAc (100 mL), washed with brine (10 mL), dried over Na₂SO₄, and concentrated in vacuo. The residue was subjected to silica gel chromatography using an ELSD eluting with hexanes-EtOAc to provide 61A. LCMS (m/z): 231.61 [M+H]⁺; t_{R}=1.09 min. on LC/MS Method A.

Synthesis of (R)-tert-butyl (2-methyl-1-oxohexan-2-yl)carbamate (61B). To a solution of 61A (2.1 g, 9.0 mmol) in DCM (100 mL) was added Dess-Martin periodinane (5.7 g, 14 mmol). After 2 h the reaction was quenched with sat. Na₂S₂O_{3(aq)} (75 mL). The mixture was separated and the aqueous layer was extracted with DCM (100 mL). The combined organics were washed with water (100 mL) and brine (100 mL), dried over Na₂SO₄, then filtered and concentrated in vacuo. The residue was subjected to silica gel chromatography using an ELSD eluting with hexanes-EtOAc to provide 61B. LCMS (m/z): 173.75 [M+H-(t-Bu)]⁺; t_{R}=1.18 min. on LC/MS Method A.

Synthesis of (R)-tert-butyl (1-(benzylamino)-2-methylhexan-2-yl)carbamate (61C). To a solution of 61B (1.9 g, 8.4 mmol) in dry MeOH (50 mL) was added benzylamine (1.0 mL, 8.35 mmol). After 18 h, sodium borohydride (500 mg, 13 mmol) was added portionwise. At 60 minutes, the mixture was concentrated in vacuo. The resulting residue was dissolved in EtOAc (50 mL), washed with 1M NaOH_{(aq)} (50 mL), 10% Rochelle's salt aq. solution (50 mL, solid supplied by Sigma-Aldrich), and brine (50 mL), dried over Na₂SO₄, then filtered and concentrated in vacuo to afford 61C. LCMS (m/z): 321.03 [M+H]⁺; t_{R}=0.94 min. on LC/MS Method A.

Synthesis of (R)-tert-butyl (1-(N-benzylacetamido)-2-methylhexan-2-yl)carbamate (61D). To a solution of 61C (2.2 g, 6.9 mmol) in THF (50 mL) was added N,N-diisopropylethylamine (2.4 mL, 14 mmol) followed by acetyl chloride (0.75 mL, 11 mmol). After 60 minutes, the mixture was diluted with EtOAc (150 mL), washed with sat. NaHCO_{3(aq)} (100 mL) and brine (100 mL), dried over Na₂SO₄, then filtered and concentrated in vacuo. The residue was subjected to silica gel chromatography eluting with hexanes-EtOAc to provide 61D. LCMS (m/z): 362.82 [M+H]⁺; t_{R}=1.32 min. on LC/MS Method A.

Synthesis of (R)—N-(2-amino-2-methylhexyl)acetamide (61E). To a solution of 61D (2.0 g, 5.4 mmol) in EtOH (55 mL) and hydrochloric acid solution in dioxane (4M, 2 mL) that was purged with Ar, was added palladium hydroxide on carbon (20 wt %, 2.0 g). The mixture was purged with H₂ and heated to 60° C. After 24 h, the reaction mixture was filtered through Celite, rinsed with EtOAc, and concentrated in vacuo to afford 61E as a HCl salt. LCMS (m/z): 172.92 [M+H]⁺; t_{R}=0.50 min. on LC/MS Method A.

Synthesis of (R)—N-(2-((2-((2,4-dimethoxybenzyl)amino)-7-fluoropyrido[3,2-d]pyrimidin-4-yl)amino)-2-methylhexyl)acetamide (61F). To a solution of 2,4-dichloropyrido[3,2-d]pyrimidine (30 mg, 0.15 mmol) in THF (10 mL) was added 61E (25 mg, 0.15 mmol) and N,N-diisopropylethylamine (0.08 mL, 0.44 mmol). After stirring at 80° C. for 18 h, 2,4-dimethoxybenzylamine (0.1 mL, 0.73 mmol) was added and the mixture heated to 100° C. After 18 h, the reaction was cooled to rt, diluted with EtOAc, washed with water and brine, dried over Na₂SO₄, and concentrated in vacuo. The residue was subjected to silica gel chromatography eluting with EtOAc-MeOH to provide 61F. ^{\}LCMS (m/z): 467.24 [M+H]⁺; t_{R}=1.02 min. on LC/MS Method A.

Synthesis of (R)—N-(2-((2-amino-7-fluoropyrido[3,2-d]pyrimidin-4-yl)amino)-2-methylhexyl)acetamide (61). To 61F (33 mg, 0.07 mmol) was added TFA (3 mL). After 60 minutes, the mixture was concentrated in vacuo and co-evaporated with MeOH (×3). The residue was suspended in MeOH, filtered, and concentrated in vacuo to provide 61 as a TFA salt. ¹H NMR (400 MHz, MeOH-d₄) δ 8.63 (dd, J=4.4, 1.4 Hz, 1H), 7.84 (dd, J=8.5, 1.4 Hz, 1H), 7.76 (dd, J=8.5, 4.4 Hz, 1H), 3.95 (d, J=14.0 Hz, 1H), 3.57 (d, J=14.0 Hz, 1H), 2.25-2.12 (m, 1H), 1.95 (s, 3H), 1.95-1.86 (m, 1H), 1.54 (s, 3H), 1.41-1.32 (m, 4H), 0.95-0.90 (m, 3H). ¹⁹F NMR (377 MHz, MeOH-d₄) δ −77.77. LCMS (m/z): 317.24 [M+H]⁺; t_{R}=0.71 min. on LC/MS Method A.

### Example 62

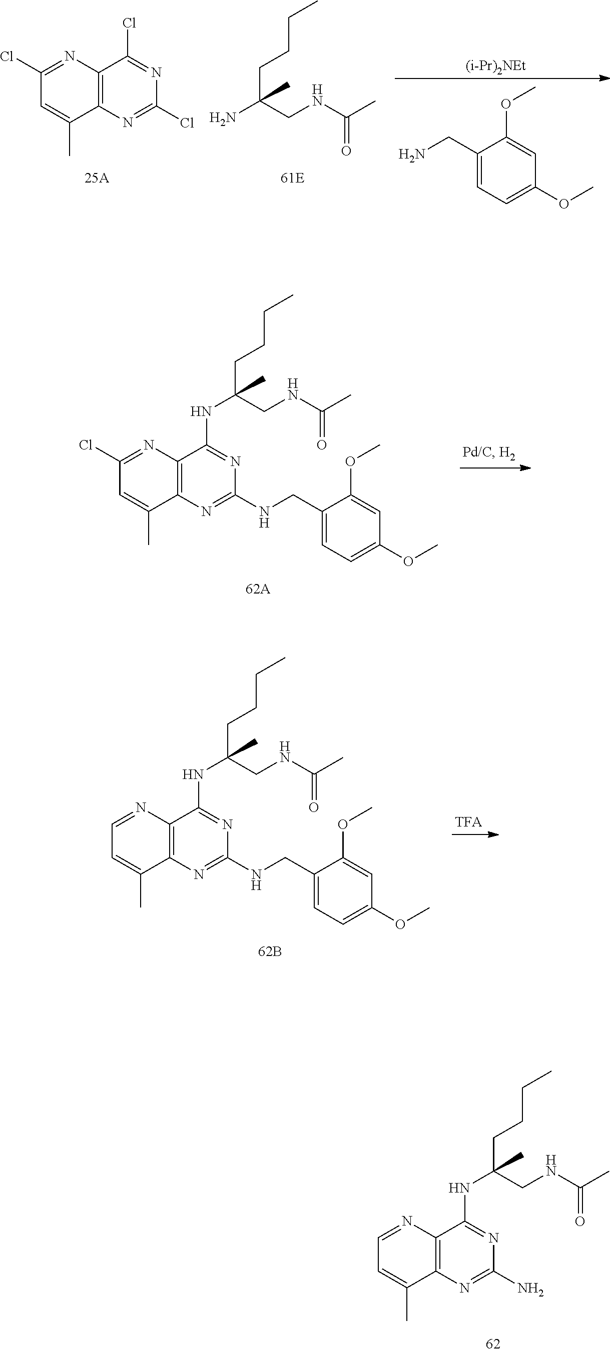

Synthesis of (R)—N-(2-((6-chloro-2-((2,4-dimethoxybenzyl)amino)-8-methylpyrido[3,2-d]pyrimidin-4-yl)amino)-2-methylhexyl)acetamide (62A). To a solution of 25A (37 mg, 0.15 mmol) in THF (5 mL) was added 61E (25 mg, 0.15 mmol) and N,N-diisopropylethylamine (0.4 mL, 0.43 mmol). After stirring at 80° C. for 18 h, 2,4-dimethoxybenzylamine (0.1 mL, 0.63 mmol) was added and the mixture was heated to 100° C. After 18 h, the reaction was cooled to rt, diluted with EtOAc, washed with water (50 mL) and brine (50 mL), dried over Na₂SO₄, then filtered and concentrated in vacuo. The residue was subjected to silica gel chromatography eluting with EtOAc-MeOH to provide 62A (49 mg, 75%). LCMS (m/z): 515.17 [M+H]⁺; t_{R}=0.86 min. on LC/MS Method A.

Synthesis of (R)—N-(2-((2-((2,4-dimethoxybenzyl)amino)-8-methylpyrido[3,2-d]pyrimidin-4-yl)amino)-2-methylhexyl)acetamide (62B). To a solution of 62A (49 mg, 0.1 mmol) in EtOAc (4 mL) and EtOH (4 mL) that was purged with Ar, was added Pd/C (Degussa 10 wt %, 25 mg). The mixture was then purged with H₂ and heated to 70° C. After 1 h, the reaction was allowed to cool to rt, purged with Ar, filtered through Celite, rinsed with EtOAc (50 mL), and concentrated in vacuo to provide 62B (46 mg, 100%). LCMS (m/z): 481.25 [M+H]⁺; t_{R}=1.10 min. on LC/MS Method A.

Synthesis of (R)-2-((2-amino-8-methylpyrido[3,2-d]pyrimidin-4-yl)amino)hexan-1-ol (62). To 62B (46 mg, 0.1 mmol) was added TFA (3 mL). After 18 h, the mixture was concentrated in vacuo and co-evaporated with MeOH (3×10 mL). The residue was suspended in 10 mL MeOH, filtered, and concentrated in vacuo to provide 62 as a TFA salt. ¹H NMR (400 MHz, MeOH-d₄) δ 8.48 (d, J=4.6 Hz, 1H), 7.61 (dd, J=4.7, 1.0 Hz, 1H), 3.95 (d, J=14.0 Hz, 1H), 3.56 (d, J=14.0 Hz, 1H), 2.52 (d, J=0.8 Hz, 3H), 2.18 (ddd, J=13.5, 11.3, 4.5 Hz, 1H), 1.95 (s, 3H), 1.89 (ddd, J=13.5, 11.6, 4.8 Hz, 1H), 1.54 (s, 3H), 1.42-1.31 (m, 5H), 0.96-0.89 (m, 4H). ¹⁹F NMR (377 MHz, MeOH-d₄) δ −77.85. LCMS (m/z): 331.16 [M+H]⁺; t_{R}=0.79 min. on LC/MS Method A.

### Example 63

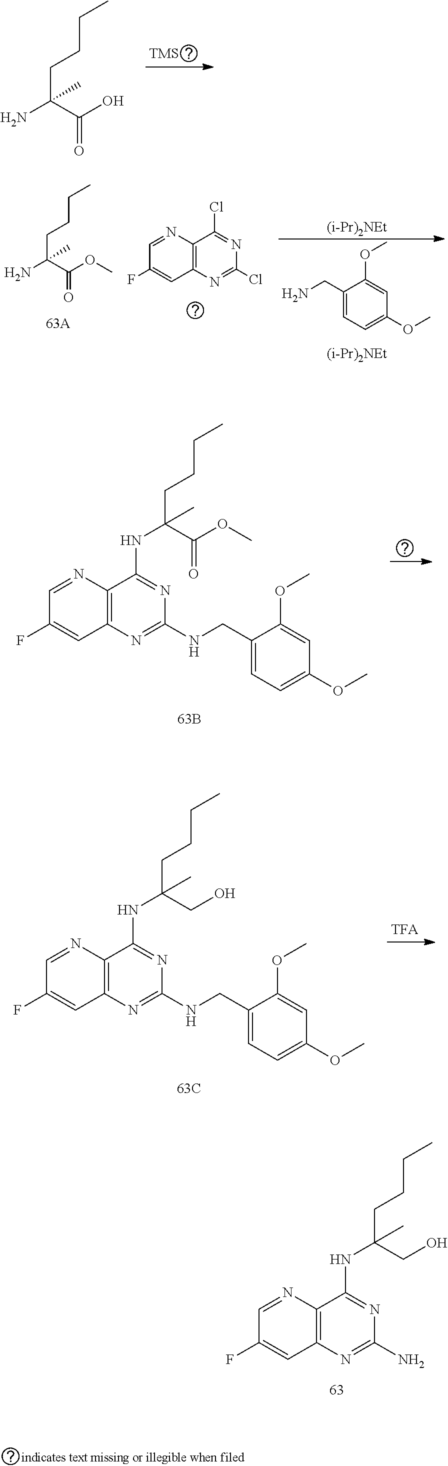

Synthesis of methyl 2-amino-2-methylhexanoate (63A). To a mixture of (2R)-2-amino-2-methylhexanoic acid hydrochloride (50 mg, 0.28 mmol) and (2S)-2-amino-2-methylhexanoic acid hydrochloride (50 mg, 0.28 mmol) in MeOH (5.0 mL) was added (trimethylsilyl) diazomethane in hexanes (2 M, 0.41 mL, 0.83 mmol) dropwise. After 6 h, the reaction was quenched with AcOH (100 μL). The mixture was concentrated in vacuo to provide 63A that was used without further isolation. LCMS (m/z): 159.91 [M+H]⁺; t_{R}=0.57 min. on LC/MS Method A.

Synthesis of methyl 2-((2-((2,4-dimethoxybenzyl)amino)-7-fluoropyrido[3,2-d]pyrimidin-4-yl)amino)-2-methylhexanoate (63B). To a solution of 84E (120 mg, 0.55 mmol) in THF (5 mL) was added 63A (88 mg, 0.55 mmol) and N,N-diisopropylethylamine (0.3 mL, 1.7 mmol). After stirring at 80° C. for 18 h, the reaction was cooled to rt, diluted with EtOAc (50 mL), washed with water (50 mL) and brine (50 mL), dried over Na₂SO₄, then filtered and concentrated in vacuo. The crude residue was then diluted with THF (10 mL) and 2,4-dimethoxybenzylamine (0.4 mL, 2.6 mmol) and N,N-diisopropylethylamine (0.3 mL, 1.7 mmol) were added. After stirring at 100° C. for 18 h, the reaction was cooled to rt, diluted with EtOAc (50 mL), washed with water and brine, dried over Na₂SO₄, then filtered and concentrated in vacuo. The residue was subjected to silica gel chromatography eluting with hexanes-EtOAc to provide 63B. ¹H NMR (400 MHz, Chloroform-d) δ 8.14 (d, J=2.5 Hz, 1H), 7.36 (s, 1H), 7.28-7.24 (m, 2H), 6.46 (d, J=2.3 Hz, 1H), 6.41 (dd, J=8.3, 2.4 Hz, 1H), 4.54 (dd, J=6.2, 2.7 Hz, 2H), 3.84 (s, 3H), 3.78 (s, 3H), 3.69 (s, 3H), 2.27-2.16 (m, 1H), 2.02 (s, 1H), 1.71 (s, 3H), 1.34-1.23 (m, 5H), 0.88 (t, J=6.9 Hz, 3H). ¹⁹F NMR (376 MHz, Chloroform-d) δ −121.51 (d, J=422.9 Hz). LCMS (m/z): 472.21 [M+H]⁺; t_{R}=0.91 min. on LC/MS Method A.

Synthesis of 2-((2-((2,4-dimethoxybenzyl)amino)-7-fluoropyrido[3,2-d]pyrimidin-4-yl)amino)-2-methylhexan-1-ol (63C). To a solution of 63B (104 mg, 0.22 mmol) in THF (5 mL) was added lithium aluminum hydride in Et₂O (2M, 0.30 mL, 0.60 mmol). After 5 h the reaction was quenched with H₂O (1 mL) and 2M NaOH_{(aq)}, and then filtered. The mother liquor was then diluted with EtOAc (30 mL), washed with sat. Rochelle's salt solution (25 mL), H₂O (25 mL), and brine (25 mL), dried over Na₂SO₄, then filtered and concentrated in vacuo. The residue was subjected to silica gel chromatography eluting with hexanes-EtOAc to provide 63C. ¹H NMR (400 MHz, Chloroform-d) δ 8.12 (d, J=2.5 Hz, 1H), 7.32 (s, 1H), 7.28 (s, 1H), 6.46 (d, J=2.4 Hz, 1H), 6.42 (dd, J=8.2, 2.4 Hz, 1H), 4.57-4.52 (m, 2H), 3.84 (s, 3H), 3.79 (s, 4H), 3.75 (s, 2H), 1.92 (d, J=14.1 Hz, 1H), 1.74 (t, J=12.6 Hz, 1H), 1.40-1.37 (m, 3H), 1.32 (td, J=13.4, 12.4, 6.3 Hz, 4H), 0.91 (t, J=7.0 Hz, 3H). ¹⁹F NMR (377 MHz, Chloroform-d) δ −121.34. LCMS (m/z): 444.20 [M+H]⁺; t_{R}=0.94 min. on LC/MS Method A.

Synthesis of 2-((2-amino-7-fluoropyrido[3,2-d]pyrimidin-4-yl)amino)-2-methylhexan-1-ol (63). To 63C (22 mg, 0.05 mmol) was added TFA (3 mL). After 30 minutes, the reaction mixture was diluted with MeOH (5 mL). After stirring for 18 h, the mixture was filtered and concentrated in vacuo. Co-evaporation with MeOH (×3) provided 63 as a TFA salt. ¹H NMR (400 MHz, MeOH-d₄) δ 8.53 (d, J=2.4 Hz, 1H), 8.20 (s, 1H), 7.65 (dd, J=8.8, 2.4 Hz, 1H), 3.95 (s, 1H), 3.70 (d, J=11.2 Hz, 1H), 2.09 (ddd, J=13.9, 10.9, 5.3 Hz, 1H), 1.96-1.86 (m, 1H), 1.53 (s, 3H), 1.42-1.28 (m, 6H), 0.95-0.87 (m, 3H). ¹⁹F NMR (377 MHz, MeOH-d₄) δ −77.47, −118.23 (d, J=8.6 Hz). LCMS (m/z): 294.12 [M+H]⁺; t_{R}=0.68 min. on LC/MS Method A.

### Example 64

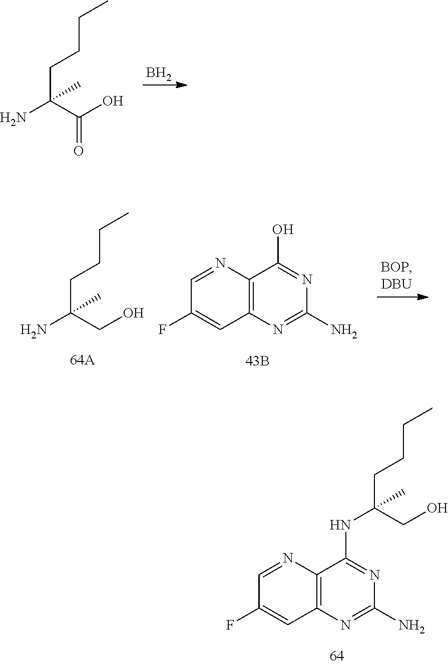

Synthesis of (S)-2-amino-2-methylhexan-1-ol (64A). To (2S)-2-amino-2-methylhexanoic acid hydrochloride (250 mg, 1.4 mmol, supplied by Astatech) in THF (5 mL) was added borane-tetrahydrofuran complex solution in THF (1M, 5.5 mL) dropwise over 5 minutes. After 24 h, the reaction was quenched with MeOH (1 mL) and concentrated in vacuo. The residue was taken up in DCM (10 mL), filtered, and concentrated in vacuo to provide crude 64A. LCMS (m/z): 131.92 [M+H]⁺; t_{R}=0.57 min. on LC/MS Method A.

Synthesis of (S)-2-((2-amino-7-fluoropyrido[3,2-d]pyrimidin-4-yl)amino)-2-methylhexan-1-ol (64). To a solution of 43B (140 mg, 78 mmol) and 64A (125 mg, 0.95 mmol) in NMP (7.5 mL), was added DBU (0.35 mL, 2.4 mmol) followed by BOP (419 mg, 0.95 mmol). After 16 h, the reaction mixture was subjected to prep HPLC (Gemini 10u C18110A, AXIA; 10% aq. acetonitrile-50% aq. acetonitrile with 0.1% TFA, over 20 min. gradient) to provide, after removal of volatiles in vacuo, 64 as a TFA salt. ¹H NMR (400 MHz, MeOH-d₄) δ 8.55 (d, J=2.4 Hz, 1H), 8.22 (s, 1H), 7.64 (dd, J=8.7, 2.5 Hz, 1H), 3.97 (d, J=11.2 Hz, 1H), 3.71 (d, J=11.2 Hz, 1H), 2.09 (ddd, J=13.9, 10.8, 5.2 Hz, 1H), 1.92 (ddd, J=13.6, 10.9, 5.4 Hz, 1H), 1.54 (s, 4H), 1.40-1.31 (m, 5H), 1.00-0.85 (m, 3H). ¹⁹F NMR (377 MHz, MeOH-d₄) δ −77.62, −118.22 (d, J=8.7 Hz). LCMS (m/z) 294.09 [M+H]⁺; t_{R}=0.79 min. on LC/MS Method A.

### Example 65

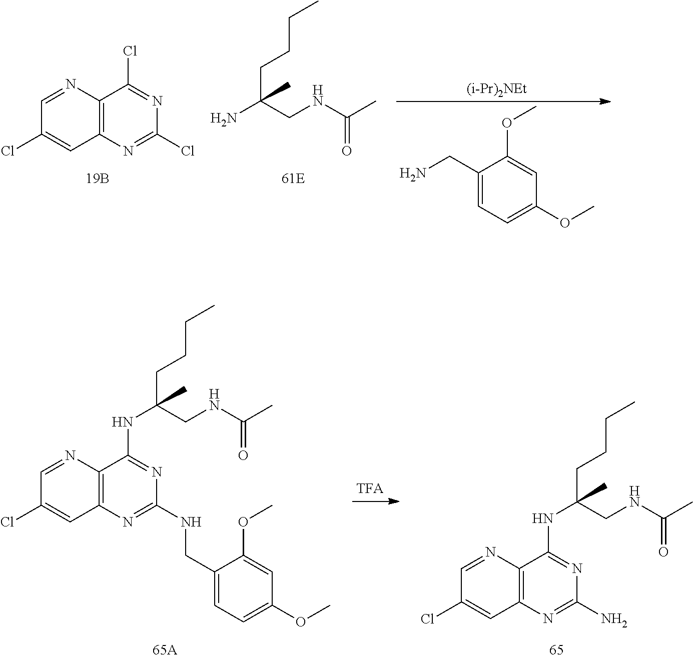 [SMILES: "(i-Pr)2NEt" ; Clc1cnc2c(Cl)nc(Cl)nc2c1 ; CCCC[C@@](C)(N)CNC(C)=O ; COc1ccc(CN)c(OC)c1 ; CCCC[C@](C)(CNC(C)=O)Nc1nc(NCc2ccc(OC)cc2OC)nc2cc(Cl)cnc12 ; CCCC[C@](C)(CNC(C)=O)Nc1nc(N)nc2cc(Cl)cnc12]

Synthesis of (R)—N-(2-((2-amino-7-chloropyrido[3,2-d]pyrimidin-4-yl)amino)-2-methylhexyl)acetamide (65A). To a solution of 19B (112 mg, 0.48 mmol) in THF (5 mL) was added 61E (100 mg, 0.48 mmol) and N,N-diisopropylethylamine (0.25 mL, 1.4 mmol). After stirring at 80° C. for 18 h, 2,4-dimethoxybenzylamine (0.75 mL, 5.0 mmol) was added and the mixture was heated to 100° C. After 18 h, the reaction was cooled to rt, diluted with EtOAc (50 mL), washed with water (50 mL) and brine (50 mL), dried over Na₂SO₄, then filtered and concentrated in vacuo. The residue was subjected to silica gel chromatography eluting with hexanes-EtOAc to provide 65A LCMS (m/z): 509.30 [M+H]⁺; t_{R}=0.89 min. on LC/MS Method A.

Synthesis of (R)—N-(2-((2-amino-7-chloropyrido[3,2-d]pyrimidin-4-yl)amino)-2-methylhexyl)acetamide (65). To 65A (21 mg, 0.04 mmol) was added TFA (3 mL). After 30 minutes, the mixture was concentrated in vacuo and the residue co-evaporated with MeOH (10 mL×3). The resulting residue was suspended in MeOH (10 mL), filtered, and concentrated in vacuo to provide 65 as a TFA salt. ¹H NMR (400 MHz, MeOH-d₄) δ 8.59 (d, J=2.1 Hz, 1H), 8.58 (s, 1H), 7.91 (d, J=2.1 Hz, 1H), 3.93 (d, J=14.0 Hz, 1H), 3.52 (d, J=14.0 Hz, 1H), 2.22-2.10 (m, 1H), 1.96 (s, 3H), 1.95-1.87 (m, 1H), 1.54 (s, 3H), 1.34 (dd, J=7.5, 3.9 Hz, 5H), 0.94-0.89 (m, 3H). ¹⁹F NMR (377 MHz, MeOH-d₄) δ −77.91. LCMS (m/z): 351.29 [M+H]⁺; t_{R}=0.69 min. on LC/MS Method A.

### Example 66

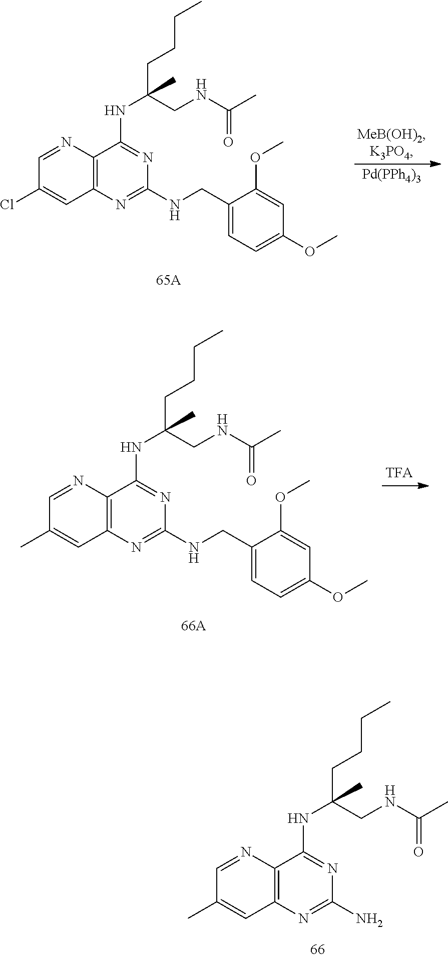

Synthesis of (R)—N-(2-((2-((2,4-dimethoxybenzyl)amino)-7-methylpyrido[3,2-d]pyrimidin-4-yl)amino)-2-methylhexyl)acetamide (66A). To 65A (128 mg, 0.26 mmol) in 1,4-dioxane (10 mL) and water (10 mL) was added methylboronic acid (61 mg, 1.0 mmol), tetrakis(triphenylphosphine)palladium(0) (51 mg, 0.05 mmol), and potassium phosphate tribasic (163 mg, 0.77 mmol). The reaction mixture was heated to 150° C. in a microwave reactor for 30 minutes. The reaction mixture was diluted with water (50 mL) and extracted with EtOAc (3×25 mL). The combined organics were washed with water (50 mL) and brine (50 mL), dried over Na₂SO₄, and concentrated in vacuo. The residue was subjected to silica gel chromatography eluting with EtOAc-MeOH, to provide 66A. LCMS (m/z): 481.30 [M+H]⁺; t_{R}=0.89 min. on LC/MS Method A.

Synthesis of (R)—N-(2-((2-amino-7-methylpyrido[3,2-d]pyrimidin-4-yl)amino)-2-methylhexyl)acetamide (66). To 66A (54 mg, 0.11 mmol) was added TFA (3 mL). After 60 minutes, the mixture was concentrated in vacuo and co-evaporated with MeOH (10 mL×3). The resulting residue was suspended in MeOH (10 mL), filtered, and concentrated in vacuo to provide 66 as a TFA salt. ¹H NMR (400 MHz, MeOH-d₄) δ 8.48 (d, J=1.8 Hz, 1H), 7.64 (s, 1H), 3.94 (d, J=14.0 Hz, 1H), 3.57 (d, J=13.9 Hz, 1H), 2.50 (s, 3H), 2.17 (ddd, J=13.4, 11.4, 4.7 Hz, 1H), 1.95 (s, 3H), 1.88 (ddd, J=16.1, 8.9, 4.4 Hz, 1H), 1.53 (s, 3H), 1.39-1.29 (m, 4H), 0.97-0.86 (m, 3H). ¹⁹F NMR (377 MHz, MeOH-d₄) δ −77.86. LCMS (m/z): 331.34 [M+H]⁺; t_{R}=0.93 min. on LC/MS Method A.

### Example 67

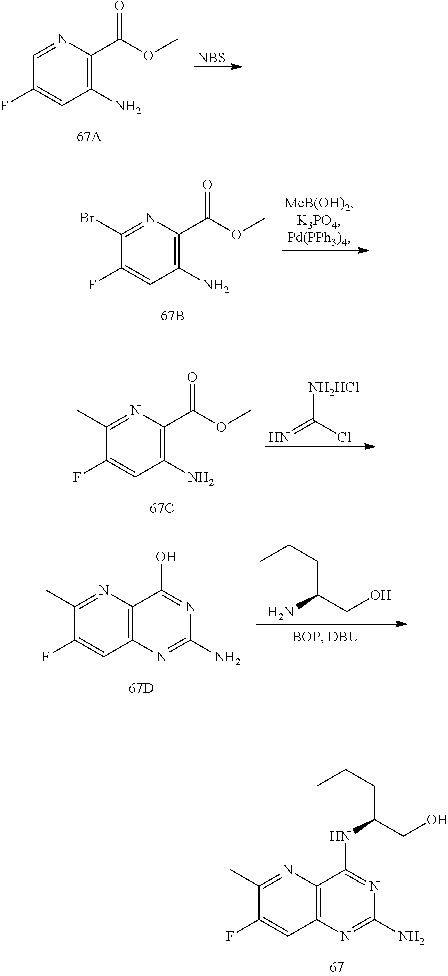

Synthesis of methyl 3-amino-6-bromo-5-fluoropicolinate (67B). To a solution of methyl 3-amino-5-fluoropicolinate 67A (270 mg, 2 mmol, 1.0 equiv., supplied by Astatech, Inc.) in acetonitrile (2 mL, 0.1M solution) was added NBS (311 mg, 2.2 mmol, 1.1 equiv.) over 2 minutes at rt. After 18 h, the reaction was quenched with water (50 mL) and the mixture was extracted with EtOAc (50 mL), washed with water (50 mL) and brine (50 mL), then dried over Na₂SO₄, filtered and then concentrated in vacuo. The residue was subjected to silica column chromatography eluting with 0% to 100% EtOAc in hexanes to provide 67B. LCMS (m/z): 250.1 [M+H]⁺; t_{R}=0.71 min. on LC/MS Method A.

Synthesis of methyl 3-amino-5-fluoro-6-methylpicolinate (67C). Methyl 3-amino-6-bromo-5-fluoropicolinate 67B (50 mg, 0.2 mmol, 1 equiv.) in a microwave vial was treated with dioxane (2 mL) and water (2 mL), along with methylboronic acid (36.05 mg, 0.06 mmol, 3 equiv.), potassium phosphate tribasic (85.23 mg, 0.4 mmol, 2 equiv.) and palladium(0) tetrakis(triphenylphosphine) (46.4 mg, 0.04 mmol, 0.2 equiv.). The mixture was heated to 120° C. for 20 min. and the reaction mixture was partitioned between EtOAc (20 mL) and H₂O (20 mL). The organic layers were combined, dried over MgSO₄ then filtered and volatiles removed in vacuo. The resulting residue was subjected to silica gel chromatography eluting with 0-100% EtOAc in hexanes to provide 67C. LCMS (m/z): 184.88 [M+H]⁺; t_{R}=0.54 min. on LC/MS Method A.

Synthesis of 2-amino-7-fluoro-6-methylpyrido[3,2-d]pyrimidin-4-ol (67D). A flask containing methyl 3-amino-5-fluoro-6-methylpicolinate 67C (95 mg, 0.52 mmol) was treated with chloroformamidine hydrochloride (118 mg, 1.03 mmol, supplied by Oakwood Scientific, Inc.). The mixture was heated to 160° C. overnight. The mixture was allowed to cool to rt, diluted with EtOAc (100 mL), filtered, and then the collected solids washed with water (50 mL) and diethyl ether (50 mL). The solid was allowed to air dry to furnish 67D which was used without further purification. LCMS (m/z): 195.03 [M+H]⁺; t_{R}=0.31 min. on LC/MS Method A.

Synthesis of (S)-2-((2-amino-7-fluoro-6-methylpyrido[3,2-d]pyrimidin-4-yl)amino)pentan-1-ol (67). To a flask containing 2-amino-7-fluoro-6-methylpyrido[3,2-d]pyrimidin-4-ol 67D (5 mg, 0.026 mmol) was added DMF (2 mL) along with 1,8-diazabicyclo[5.4.0]undec-7-ene solution 1M in THF (0.01 mL, 0.08 mmol), (benzotriazol-1-yloxy)tris(dimethylamino)phosphonium hexafluorophosphate (22.78 mg, 0.05 mmol) and (S)-(+)-2-amino-1-pentanol, (10.63 mg, 0.1 mmol). The reaction was allowed to stir overnight and then subjected to HPLC (10% to 70% MeCN in water with 0.1% TFA using a Hydro-RP column) to provide, after removal of volatiles in vacuo, 67 as its TFA salt; t_{R}=0.57 min. on LC/MS Method A. ¹H NMR (400 MHz, MeOH-d₄) δ 7.52 (d, J=9.4 Hz, 1H), 4.54 (s, 1H), 3.73 (d, J=5.3 Hz, 2H), 2.61 (d, J=2.9 Hz, 3H), 1.71 (q, J=7.6 Hz, 2H), 1.49-1.37 (m, 1H), 1.29 (s, 5H), 0.97 (t, J=7.4 Hz, 3H). ¹⁹F NMR (377 MHz, MeOH-d₄) δ −77.42; LCMS (m/z): 280.1 [M+H]⁺

### Example 68

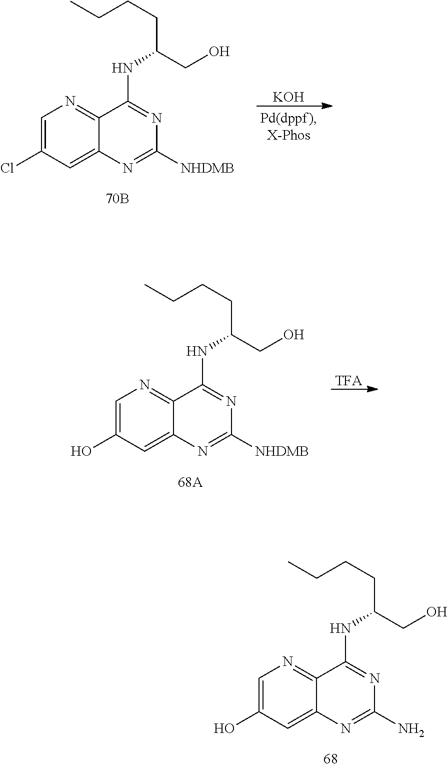

Synthesis of (R)-2-((2,4-dimethoxybenzyl)amino)-4-((1-hydroxyhexan-2-yl)amino)pyrido[3,2-d]pyrimidin-7-ol (68A). Into a microwave vial containing (R)-2-((7-chloro-2-((2,4-dimethoxybenzyl)amino)pyrido[3,2-d]pyrimidin-4-yl)amino)hexan-1-ol 70B (22 mg, 0.049 mmol, 1 equiv.) was added 2-(dicyclohexylphosphino)-2′,4′,6′-triisopropylbiphenyl (2.35 mg, 0.01 mmol), tris(dibenzylideneacetone)dipalladium(0) (0.9 mg, 0.005 mmol, 20 mol %) along with dioxane (2.5 mL) and KOH_{(aq)} (1 mL, 0.08M). The mixture was heated to 150° C. for 30 min. in a microwave reactor. The reaction mixture was partitioned between EtOAc (50 ml) and H₂O (50 mL). The organic layer was separated, dried over MgSO₄, filtered and concentrated in vacuo. The crude material 68A was used without further purification. LCMS (m/z): 428.2 [M+H]⁺; t_{R}=0.78 min. on LC/MS Method A.

Synthesis of (R)-2-amino-4-((1-hydroxyhexan-2-yl)amino)pyrido[3,2-d]pyrimidin-7-ol (68). A solution of (R)-2-((2,4-dimethoxybenzyl)amino)-4-((1-hydroxyhexan-2-yl)amino)pyrido[3,2-d]pyrimidin-7-ol 68A (21 mg, 0.05 mmol, 1 equiv.) in DCM (2 mL) was treated with TFA (0.5 mL). After 3 h the reaction mixture was concentrated under reduced pressure and the residue subjected to reverse phase HPLC (10% to 70% MeCN in water with 0.1% TFA using a Hydro-RP column) to furnish, after product fraction collection and the removal of volatiles in vacuo, 68 as its TFA salt. LCMS (m/z): 278.3 [M+H]⁺; t_{R}=0.55 min. on LC/MS Method A. ¹H NMR (400 MHz, MeOH-d₄) δ 8.61-8.34 (m, 1H), 8.19-7.98 (m, 1H), 4.39 (ddd, J=18.0, 9.2, 5.3 Hz, 2H), 3.77 (dt, J=8.3, 6.5 Hz, 1H), 1.74-1.50 (m, 6H), 1.34-1.09 (m, 10H), 0.79 (tt, J=6.9, 1.3 Hz, 6H), 0.59 (d, J=5.6 Hz, 2H). ¹⁹F NMR (377 MHz, MeOH-d₄) δ −77.55

### Example 69

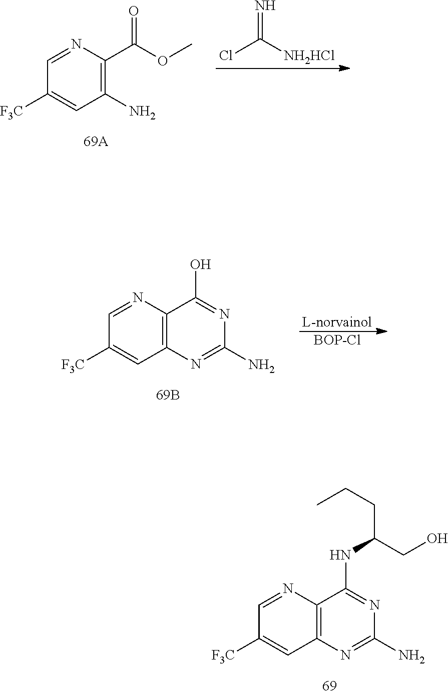

Synthesis of 2-amino-7-(trifluoromethyl)pyrido[3,2-d]pyrimidin-4-ol (69B). Methyl 3-amino-5-(trifluoromethyl)picolinate 69A (300 mg, 0.001 mol, 1 equiv., supplied by J&W Pharmlab, LLC) was treated with chloroformamidine hydrochloride (390 mg, 0.003 mmol, 2.5 equiv.) and dimethyl sulfone (1.28 g, 0.014 mol, 10 equiv.). The mixture was heated to 200° C. overnight. The reaction mixture was allowed to cool to rt, filtered, and washed with water (50 mL) and diethyl ether (50 mL). The residue was allowed to air dry to furnish 69B which was used without further purification. LCMS (m/z): 231 [M+H]⁺; t_{R}=0.48 min. on LC/MS Method A.

Synthesis of (S)-2-((2-amino-7-(trifluoromethyl)pyrido[3,2-d]pyrimidin-4-yl)amino)hexan-1-ol (69). 2-amino-7-(trifluoromethyl)pyrido[3,2-d]pyrimidin-4-ol, 69B (100 mg, 0.44 mmol, 1 equiv.) was treated with 1,8-diazabicyclo[5.4.0]undec-7-ene solution 1M in THF (0.19 mL, 1.3 mmol, 3 equiv.). (Benzotriazol-1-yloxy)tris(dimethylamino)phosphonium hexafluorophosphate (249.83 mg, 0.56 mmol, 1.3 equiv.) was added followed by (S)-(+)-2-Amino-1-pentanol (112.06 mg, 1.09 mmol, 2.5 equiv.)), and DMF (5 mL). After stirring 16 h, the reaction mixture was diluted with water (5 mL) and subjected to reverse phase HPLC (10% to 70% MeCN in water with 0.1% TFA using a Hydro-RP column) to furnish, after product fractions were collected and the volatiles removed in vacuo, the title compound 69 as its TFA salt. LCMS (m/z): 316.16 [M+H]⁺; t_{R}=0.59 min. on LC/MS Method A. ¹H NMR (400 MHz, MeOH-d₄) δ 8.94-8.53 (m, 1H), 8.01 (dd, J=1.8, 0.9 Hz, 1H), 4.45 (t, J=6.5 Hz, 1H), 3.71-3.54 (m, 2H), 3.42-3.24 (m, 2H), 2.72-2.55 (m, 2H), 1.59 (td, J=8.2, 6.6 Hz, 3H), 1.37-1.20 (m, 2H), 0.85 (t, J=7.3 Hz, 4H). ¹⁹F NMR (377 MHz, MeOH-d₄) δ −64.83, −77.69.

### Example 70 & Example 71

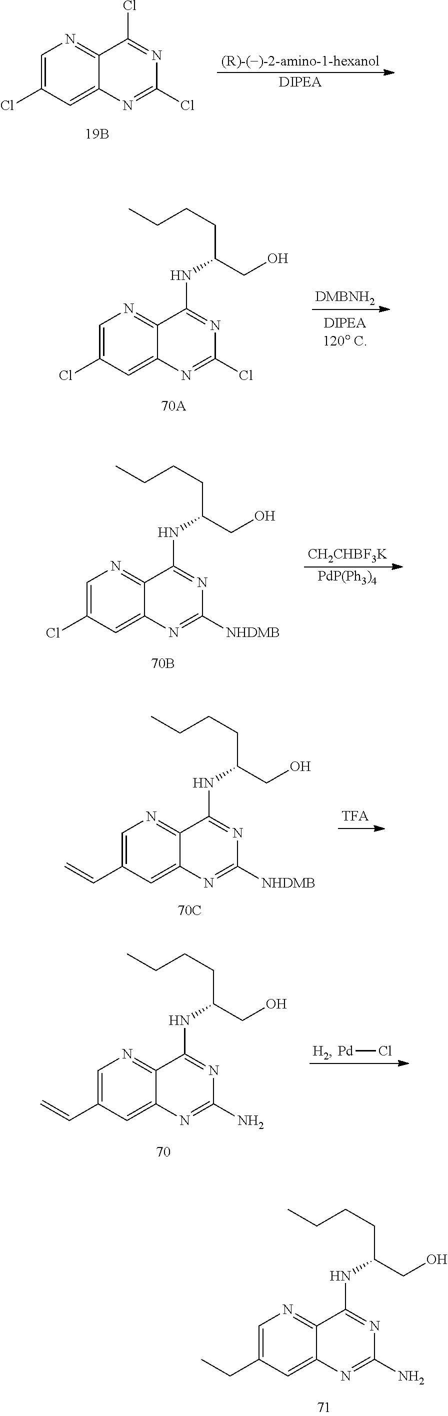

Synthesis of (R)-2-((2,7-dichloropyrido[3,2-d]pyrimidin-4-yl)amino)hexan-1-ol (70A). A solution of 2,4,7-trichloropyrido[3,2-d]pyrimidine 19B (250 mg, 1.06 mmol, 1 equiv.) in dioxane (4 mL) was treated with N,N-diisopropylethylamine (0.22 mL, 1.2 mmol, 1.5 equiv.) and (R)-(−)-2-amino-1-hexanol (312.38 mg, 3.02 mmol, 2.5 equiv.). The reaction was allowed to stir for 1 h and the product that formed, 70A, was carried forward directly into the following reaction without isolation.

Synthesis of (R)-2-((7-chloro-2-((2,4-dimethoxybenzyl)amino)pyrido[3,2-d]pyrimidin-4-yl)amino)hexan-1-ol (70B). The solution of (R)-2-((2,7-dichloropyrido[3,2-d]pyrimidin-4-yl)amino)hexan-1-ol 70A (315 mg, 1.06 mmol, 1 equiv.) prepared as described, was treated with dioxane (4 mL) followed by N,N-diisopropylethylamine (0.38 mL, 2 mmol, 2 equiv.) and 2,4-dimethoxybenzylamine (0.47 mL, 3.1 mmol, 3 equiv.). The reaction was heated at 120° C. overnight. The reaction mixture partitioned between EtOAc (50 mL) and H₂O (50 mL). The organics layer was separated, dried over Na₂SO₄, then filtered and concentrated in vacuo. The residue was subjected to silica gel chromatography eluting with 0% to 100% EtOAc in hexanes to provide the title compound 70B. LCMS (m/z): 446.9 [M+H]⁺; t_{R}=0.78 min. on LC/MS Method A.

Synthesis of (R)-2-((2-((2,4-dimethoxybenzyl)amino)-7-vinylpyrido[3,2-d]pyrimidin-4-yl)amino)hexan-1-ol (70C). A microwave vial containing (R)-2-((7-chloro-2-((2,4-dimethoxybenzyl)amino)pyrido[3,2-d]pyrimidin-4-yl)amino)hexan-1-ol 70B (50 mg, 0.11 mmol, 1 equiv.) was treated with potassium vinyltrifluoroborate (26.59 mg, 0.28 mmol, 2.5 equiv.), potassium phosphate tribasic (71.4 mg, 0.34 mmol, 3 equiv.), palladium(0) tetrakis(triphenylphosphine) (25.91 mg, 0.02 mmol, 0.2 equiv.), dioxane (2.0 mL), and water (2 mL). The mixture was heated to 150° C. for 60 min. in a microwave reactor. The reaction mixture was partitioned between EtOAc (50 mL) and H₂O (50 mL). The organic layer was separated, dried over Na₂SO₄, filtered and concentrated in vacuo to provide the crude material 70C which was used without further purification. LCMS (m/z): 438.27 [M+H]⁺; t_{R}=0.82 min. on LC/MS Method A.

Synthesis of (R)-2-((2-amino-7-vinylpyrido[3,2-d]pyrimidin-4-yl)amino)hexan-1-ol (70). A solution of (R)-2-((2-((2,4-dimethoxybenzyl)amino)-7-vinylpyrido[3,2-d]pyrimidin-4-yl)amino)hexan-1-ol, 70C (49 mg, 0.08 mmol, 1 equiv.) in DCM (2 mL) was treated with TFA (0.5 mL). After 3 h the reaction mixture was concentrated under reduced pressure and the residue subjected to reverse phase HPLC (10% to 70% MeCN in water with 0.1% TFA using a Hydro-RP column) to furnish, after product fractions were collected and removal of volatiles in vacuo, 70 as its TFA salt. LCMS (m/z): 288.17 [M+H]⁺; t_{R}=0.61 min. on LC/MS Method A. ¹H NMR (400 MHz, MeOH-d₄) δ 8.61 (d, J=1.8 Hz, 1H), 7.75-7.62 (m, 1H), 6.80 (dd, J=17.7, 11.1 Hz, 1H), 6.05 (d, J=17.7 Hz, 1H), 5.54 (d, J=11.1 Hz, 1H), 4.47-4.31 (m, 1H), 3.71-3.51 (m, 2H), 1.77-1.47 (m, 2H), 1.35-1.16 (m, 5H), 0.93-0.71 (m, 4H). ¹⁹F NMR (377 MHz, MeOH-d₄) δ −77.60.

Synthesis of (R)-2-((2-amino-7-ethylpyrido[3,2-d]pyrimidin-4-yl)amino)hexan-1-ol (71). (R)-2-((2-amino-7-vinylpyrido[3,2-d]pyrimidin-4-yl)amino)hexan-1-ol, 70 (25 mg, 0.09 mmol, 1 equiv.) was treated with Pd/C (Degussa 10 wt %, 50 mg) and EtOH (5 mL) and the mixture stirred under hydrogen. After several h the solid was filtered off and the filtrate was concentrated under reduced pressure. The residue was subjected to reverse phase HPLC (10% to 50% MeCN in water with 0.1% TFA using a Gemini C18 column) to furnish, after product fractions were collected and the removal of volatiles in vacuo, 71 as its TFA salt. LCMS (m/z): 290.42 [M+H]⁺; t_{R}=0.70 min. on LC/MS Method A. ¹H NMR (400 MHz, MeOH-d₄) δ 8.60-8.42 (m, 1H), 7.63 (td, J=1.6, 0.9 Hz, 1H), 4.61-4.44 (m, 1H), 3.82-3.63 (m, 2H), 2.85 (q, J=7.6 Hz, 2H), 1.84-1.64 (m, 3H), 1.46-1.15 (m, 9H), 0.97-0.81 (m, 4H). ¹⁹F NMR (377 MHz, MeOH-d₄) δ −77.47.

### Example 72

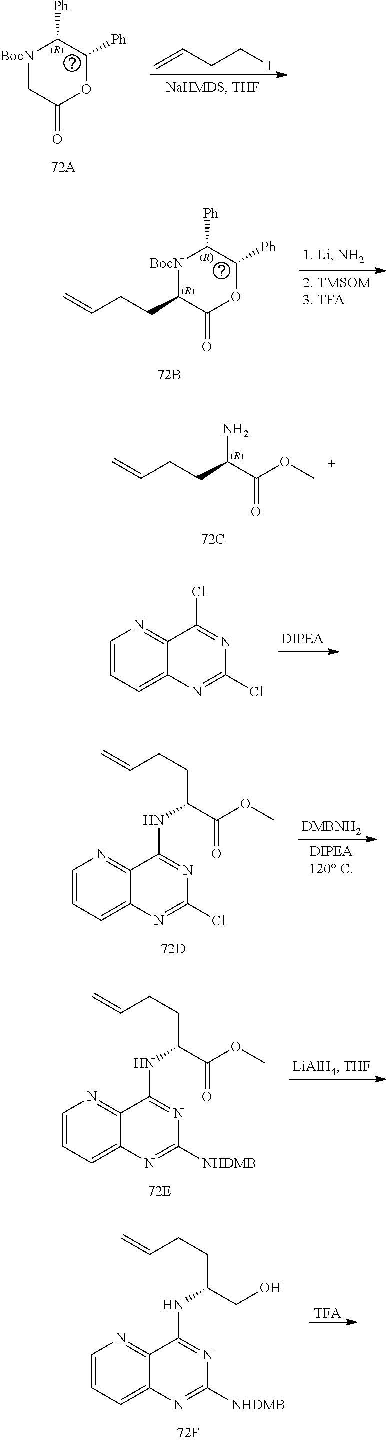

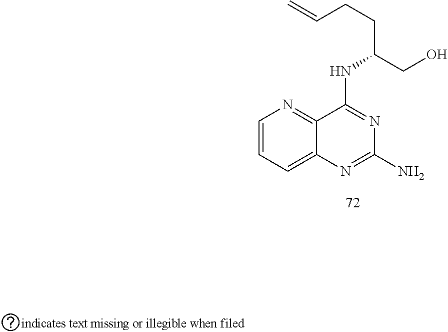 [SMILES: C=CCC[C@H](CO)Nc1nc(N)nc2cccnc12]

Synthesis of (3R,5R,6S)-tert-butyl 3-(but-3-en-1-yl)-2-oxo-5,6-diphenylmorpholine-4-carboxylate (72B). Starting with a stirred solution of (2S,3R)-tert-butyl 6-oxo-2,3-diphenylmorpholine-4-carboxylate 72A (1500 mg, 4 mmol, 1 equiv., supplied by Sigma-Aldrich) and 4-iodobutene (3862.41 mg, 0.02 mol, 5 equiv., supplied by Sigma-Aldrich) in anhydrous THF (24 mL) and HMPA (2.5 mL), cooled to −78° C., 1M sodium bis(trimethylsilyl) amide in THF (6.37 mL, 6.37 mmol, 1.5 equiv.) was added dropwise under argon. After 10 min. the reaction mixture was stirred at −40° C. for 4 h. The reaction was quenched with EtOAc (50 mL) and poured into a mixture of EtOAc (50 mL) and an aqueous solution of 1M NH₄Cl (50 mL). The organic layer was separated, washed with water (50 mL) and brine (50 mL), dried with Na₂SO₄, filtered and volatiles removed in vacuo to give a residue. The residue was subjected to silica gel chromatography eluting with 0% to 100% EtOAc in hexanes to afford the title compound 72B. LCMS (m/z): 307.98 [M+H-Boc]⁺; tR=1.28 min. on LC/MS Method A.

Synthesis of (R)-methyl 2-aminohex-5-enoate (72C). A 2-neck flask containing lithium (91.98 mg, 13.25 mmol, 15 equiv.) was cooled to −40° C. before liquid ammonia (15 mL) was added to the flask via condensation using a cold-finger apparatus. Intermediate 72B (360 mg, 0.88 mmol, 1 equiv.) in THF (2 mL) was then added. The reaction was maintained at −40° C. for 1 h, and then slowly quenched with NH₄Cl solution (5 mL), after which time it was allowed to warm to rt. The reaction was then diluted with diethyl ether (50 mL) and water (50 mL) and the diethyl ether layer separated. To the aqueous layer was then added 1 N HCl until pH 5 followed by extraction with EtOAc (50 mL). Each of the organic layers was washed with saturated NH₄Cl (50 mL) separately, and then combined, dried over MgSO₄, filtered and concentrated in vacuo. DCM (10 mL) was added to the residue followed by MeOH (1 mL), (trimethylsilyl)diazomethane (2.0M solution in hexanes) (0.29 mL, 2.20 mmol, 12 equiv.). After stirring for 1 h the reaction was concentrated under reduced pressure. The crude residue was treated with DCM (5 mL) and TFA (5 mL). After stirring for 2 h, the reaction was concentrated under reduced pressure to give 72C that was used without further purification.

Synthesis of (R)-2-((2,7-dichloropyrido[3,2-d]pyrimidin-4-yl)amino)hexan-1-ol (72D). A solution of 2,4-dichloropyrido[3,2-d]pyrimidine (110 mg, 0.55 mmol, 1.1 equiv) in dioxane (4 mL) was treated with N,N-diisopropylethylamine (0.14 mL, 0.9 mmol, 2 equiv.) and then the crude (R)-methyl 2-aminopent-4-enoate 72C (112 mg, 0.46 mmol, 1 equiv.). The reaction was allowed to stir for 1 h to provide 72D that was used directly in solution. LCMS (m/z): 307.80 [M+H]⁺; t_{R}=1.09 min. on LC/MS Method A.

Synthesis of (R)-methyl 2-((2-((2,4-dimethoxybenzyl)amino)pyrido[3,2-d]pyrimidin-4-yl)amino)hex-5-enoate (72E). The crude solution containing (R)-2-((2,7-dichloropyrido[3,2-d]pyrimidin-4-yl)amino)hexan-1-ol 72D (128 mg, 0.42 mmol, 1 equiv.) was treated with additional N,N-diisopropylethylamine (0.15 mL, 0.84 mmol, 2 equiv.) and then 2,4-dimethoxybenzylamine (0.47 mL, 0.85 mmol, 2 equiv.). The reaction was heated at 120° C. overnight. The reaction mixture was then partitioned between EtOAc (50 mL) and H₂O (50 mL). The organic layer was separated, dried over Na₂SO₄, filtered, and then concentrated in vacuo. The residue was subjected to silica gel chromatography eluting with 0% to 100% EtOAc in hexanes to provide the title compound 72E. LCMS (m/z): 438.52 [M+H]⁺; t_{R}=0.91 min. on LC/MS Method A.

Synthesis of (R)-2-((2-((2,4-dimethoxybenzyl)amino)pyrido[3,2-d]pyrimidin-4-yl)amino)hex-5-en-1-ol (72F). (R)-methyl 2-((2-((2,4-dimethoxybenzyl)amino)pyrido[3,2-d]pyrimidin-4-yl)amino)hex-5-enoate 72E (43 mg, 0.1 mmol, 1 equiv.) was dissolved in THF (5 mL) and 1M lithium aluminum hydride in diethyl ether (0.29 mL, 0.29 mmol, 3 equiv.) was added. The reaction mixture was stirred at rt for 2 h. The reaction mixture was quenched with water (50 mL) and extracted with EtOAc (50 mL). The organic layer was dried over Na₂SO₄, filtered, and then concentrated in vacuo. The crude residue 72F (40 mg) was then used without further purification. LCMS (m/z): 410.52 [M+H]⁺; t_{R}=0.85 min. on LC/MS Method A.

Synthesis of (R)-2-((2-aminopyrido[3,2-d]pyrimidin-4-yl)amino)hex-5-en-1-ol (72). (R)-2-((2-((2,4-dimethoxybenzyl)amino)pyrido[3,2-d]pyrimidin-4-yl)amino)hex-5-en-1-ol 72F (40 mg, 0.09 mmol, 1 equiv.) was treated with DCM (2 mL) and TFA (0.5 mL). After 3 h the reaction mixture was concentrated under reduced pressure and subjected to reverse phase HPLC (10% to 70% MeCN in water with 0.1% TFA using a Hydro-RP column) to furnish, after collection of product fractions and removal of volatiles in vacuo, 72 as its TFA salt. LCMS (m/z): 260.14 [M+H]⁺; t_{R}=0.58 min. on LC/MS Method A. ¹H NMR (400 MHz, MeOH-d₄) δ 8.66 (ddd, J=10.3, 4.2, 1.5 Hz, 1H), 7.94-7.65 (m, 2H), 5.86 (ddt, J=16.9, 10.3, 6.7 Hz, 1H), 5.15-4.90 (m, 2H), 4.63-4.43 (m, 1H), 2.29-2.06 (m, 2H), 2.00-1.71 (m, 2H). ¹⁹F NMR (377 MHz, Methanol-d₄) δ −77.31, −77.69.

### Example 73

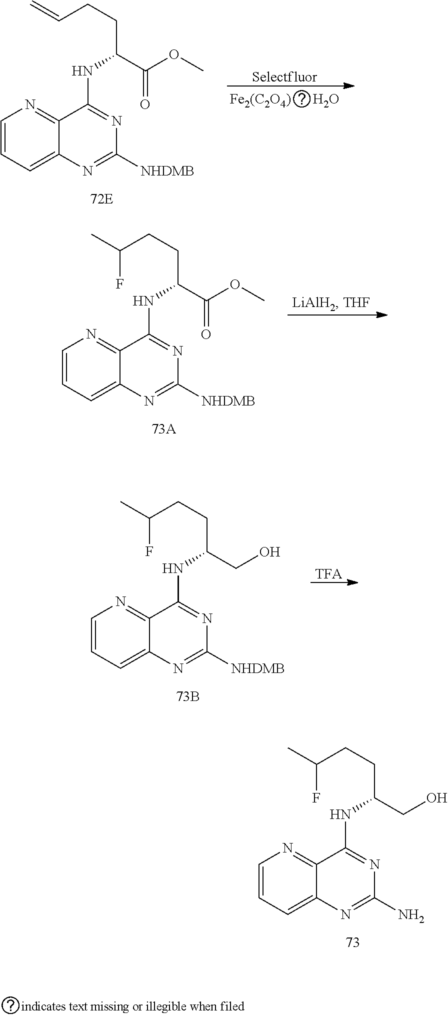

Synthesis of (2R)-methyl 2-((2-((2,4-dimethoxybenzyl)amino)pyrido[3,2-d]pyrimidin-4-yl)amino)-5-fluorohexanoate (73A). Iron (III) oxalate hexahydrate (172 mg, 0.36 mmol, 2 equiv.) was stirred in water (10 mL) until completely dissolved (typically 1-2 h). The clear yellow solution was cooled to 0° C. and degassed for 10 min. Selectfluor (126 mg, 0.36 mmol, 2 equiv.) and MeCN (5 mL) were added to the reaction mixture. A solution of (R)-methyl 2-((2-((2,4-dimethoxybenzyl)amino)pyrido[3,2-d]pyrimidin-4-yl)amino)hex-5-enoate 72E (78 mg, 0.18 mmol, 1 equiv.) in MeCN (5 mL) was added to the reaction mixture followed by sodium borohydride (23.6 mg, 0.62 mmol, 3.5 equiv.) at 0° C. After 2 min, the reaction mixture was treated with an additional portion of NaBH₄ (24 mg, 0.62 mmol, 3.5 equiv.). The resulting mixture was stirred for 30 min. and then quenched by the addition of 28-30% aqueous NH₄OH (4 mL). The mixture was extracted with 10% MeOH in CH₂Cl₂ and the organic layer was dried over Na₂SO₄, filtered and concentrated under reduced pressure. The residue was subjected to silica gel chromatography eluting with 0% to 100% EtOAc in hexanes, to provide 73A. LCMS (m/z): 458.63 [M+H]⁺; t_{R}=0.91 min. on LC/MS Method A.

Synthesis of (2R)-2-((2-((2,4-dimethoxybenzyl)amino)pyrido[3,2-d]pyrimidin-4-yl)amino)-5-fluorohexan-1-ol (73B). (2R)-methyl 2-((2-((2,4-dimethoxybenzyl)amino)pyrido[3,2-d]pyrimidin-4-yl)amino)-5-fluorohexanoate 73A (43 mg, 0.1 mmol, 1 equiv.) was treated with THF (5 mL) and 1M lithium aluminum hydride in ether (0.29 mL, 0.29 mmol, 3 equiv.). The reaction mixture was allowed to stir at rt for 2 h. The reaction mixture was quenched with water (50 mL) and extracted with EtOAc (50 mL). The organics were combined, dried over Na₂SO₄, and concentrated in vacuo. The crude material 73B was used without further purification. LCMS (m/z): 430.19 [M+H]⁺; t_{R}=0.82 min. on LC/MS Method A.

Synthesis of (2R)-2-((2-aminopyrido[3,2-d]pyrimidin-4-yl)amino)-5-fluorohexan-1-ol (73). (2R)-2-((2-((2,4-dimethoxybenzyl)amino)pyrido[3,2-d]pyrimidin-4-yl)amino)-5-fluorohexan-1-ol 73B (40 mg, 0.09 mmol, 1 equiv.) was treated with DCM (2 mL) and TFA (0.5 mL). After 3 h the reaction mixture was concentrated under reduced pressure and the residue subjected to reverse phase HPLC (10% to 70% MeCN in water with 0.1% TFA using a Hydro-RP column) to furnish, after collection of product fractions and removal of volatiles in vacuo, 73 as its TFA salt. LCMS (m/z): 280.12 [M+H]⁺; t_{R}=0.59 min. on LC/MS Method A. ¹H NMR (400 MHz, Methanol-d₄) δ 8.64 (dd, J=4.3, 1.4 Hz, 1H), 7.84 (dd, J=8.5, 1.4 Hz, 1H), 4.63-4.50 (m, 1H), 4.47 (t, J=6.0 Hz, 1H), 4.35 (t, J=6.0 Hz, 1H), 3.74 (d, J=5.3 Hz, 2H), 1.89-1.61 (m, 4H), 1.60-1.39 (m, 2H). ¹⁹F NMR (377 MHz, Methanol-d₄) δ −77.66, −220.85 (ddd, J=47.6, 25.5, 22.1 Hz).

### Example 74

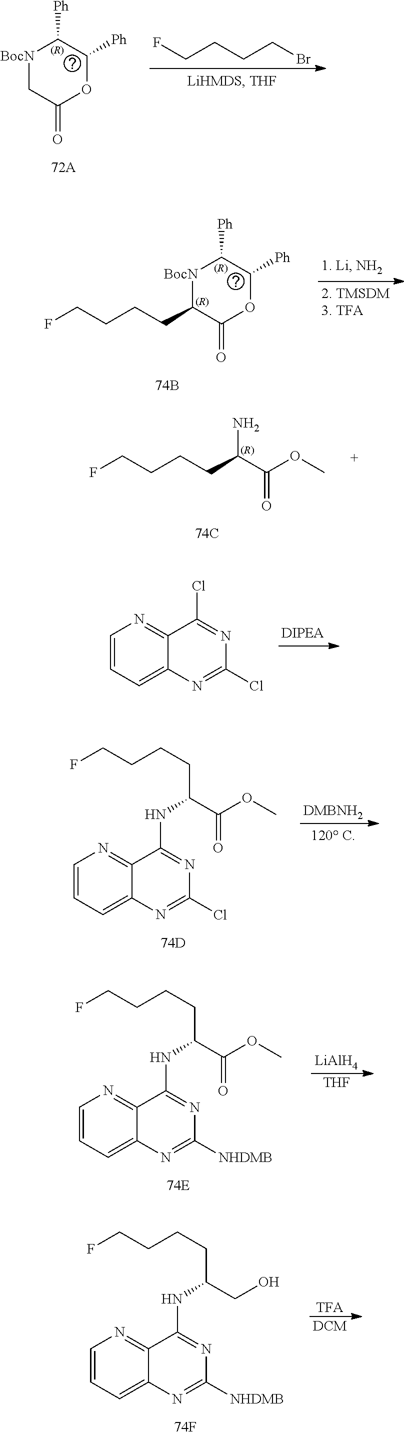

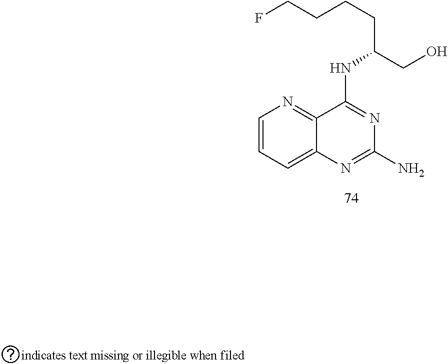 [SMILES: Nc1nc(N[C@@H](CO)CCCCF)c2ncccc2n1]

Synthesis of (3R,5R,6S)-tert-butyl 3-(4-fluorobutyl)-2-oxo-5,6-diphenylmorpholine-4-carboxylate (74B). A stirred solution of (2S,3R)-tert-butyl 6-oxo-2,3-diphenylmorpholine-4-carboxylate 72A (1000 mg, 2.8 mmol, 1 equiv.) and 1-bromo-4-fluorobutane (2.57 g, 13.5 mmol, 4.5 equiv., supplied by Sigma-Aldrich) in anhydrous THF (10 mL) and HMPA (1 mL) was cooled to −78° C. and treated dropwise with 1M Lithium bis(trimethylsilyl) amide in THF (4.2 mL, 4.2 mmol, 1.5 equiv.) under argon. After 10 min. the reaction mixture was stirred at −40° C. for 4 h. The reaction was quenched with EtOAc and poured into a mixture of EtOAc (50 mL) and an aqueous solution of NH₄Cl (50 mL, 1 M). The organic layer was separated and concentrated in vacuo to provide a crude residue which was subjected to silica gel chromatography eluting with 0% to 100% EtOAc in hexanes, to afford the title compound 74B LCMS (m/z): 328.9 [M+H-Boc]⁺; t_{R}=1.38 min. on LC/MS Method A.

Synthesis of (R)-methyl 2-amino-6-fluorohexanoate (74C). A 2-neck flask containing lithium (170 mg, 24.5 mmol, 15 equiv.) was cooled at −40° C. before liquid ammonia (15 mL) was added via a cold-finger. To the deep blue mixture (3R,5R,6S)-tert-butyl 3-(4-fluorobutyl)-2-oxo-5,6-diphenylmorpholine-4-carboxylate 74B (700 mg, 1.6 mmol, 1 equiv.) was added. The reaction mixture was maintained at this temperature for 1 h and then allowed to warm up to rt. The reaction was slowly quenched with NH₄Cl solution and diluted with diethyl ether and the organic layer separated. The aqueous layer was adjusted to pH 5 with 1N HCl and was then extracted with EtOAc. The organic layers were washed with saturated NH₄Cl, dried over MgSO₄, filtered, and concentrated under reduced pressure. The organic residues were combined and treated with DCM (10 mL) and MeOH (1 mL) along with (trimethylsilyl)diazomethane (2.0M solution in hexanes, 0.50 mL, 3.2 mmol, 4 equiv.). After 1 h the reaction mixture was concentrated under reduced pressure. The crude residue material was treated with DCM (5 mL) and TFA (5 mL). The mixture was stirred for 2 h and then concentrated under reduced pressure to provide crude 74C that was used without further purification.

Synthesis of (R)-methyl 2-((2-chloropyrido[3,2-d]pyrimidin-4-yl)amino)-6-fluorohexanoate (74D). 2,4-dichloropyrido[3,2-d]pyrimidine (163 mg, 0.82 mmol, 1.1 equiv.) was dissolved in dioxane (6 mL), N,N-diisopropylethylamine (0.53 mL, 2.9 mmol, 4 equiv.) and (R)-methyl 2-amino-6-fluorohexanoate 74C (205 mg, 0.74 mmol, 1 equiv.). The reaction mixture was stirred for 1 h and then the mixture of 74D used directly. LCMS (m/z): 326.80 [M+H]⁺; t_{R}=1.04 min. on LC/MS Method A.

Synthesis of (R)-methyl 2-((2-((2,4-dimethoxybenzyl)amino)pyrido[3,2-d]pyrimidin-4-yl)amino)-6-fluorohexanoate (74E). A solution of (R)-methyl 2-((2-((2,4-dimethoxybenzyl)amino)pyrido[3,2-d]pyrimidin-4-yl)amino)-6-fluorohexanoate 74D (243 mg, 0.74 mmol, 1 equiv.) prepared as described, was treated with 2,4-dimethoxybenzylamine (0.22 mL, 1.49 mmol, 2 equiv.). The reaction was heated at 120° C. overnight. The reaction mixture was partitioned between EtOAc (50 mL) and H₂O (50 mL). The organic layer was separated, dried over Na₂SO₄, and concentrated in vacuo. The residue was subjected to silica gel chromatography eluting with 0% to 100% EtOAc in hexanes to provide 74E. LCMS (m/z): 445.61 [M+H]⁺; t_{R}=0.87 min. on LC/MS Method A.

Synthesis of (R)-2-((2-((2,4-dimethoxybenzyl)amino)pyrido[3,2-d]pyrimidin-4-yl)amino)-6-fluorohexan-1-ol (74F). (R)-methyl 2-((2-((2,4-dimethoxybenzyl)amino)pyrido[3,2-d]pyrimidin-4-yl)amino)-6-fluorohexanoate 74E (236 mg, 0.52 mmol, 1 equiv) was treated with THF (5 mL) and 1M lithium aluminum hydride in ether (1.5 mL, 1.54 mmol, 3 equiv.). The reaction was stirred at rt. After 2 h, the reaction was quenched with water (50 mL) and extracted with EtOAc (50 mL). The organic layer was dried over Na₂SO₄, and concentrated in vacuo. The crude material 74F was used without further purification. LCMS (m/z): 430.52 [M+H]⁺; t_{R}=0.79 min. on LC/MS Method A.

Synthesis of (R)-2-((2-aminopyrido[3,2-d]pyrimidin-4-yl)amino)-6-fluorohexan-1-ol (74). (R)-2-((2-((2,4-dimethoxybenzyl)amino)pyrido[3,2-d]pyrimidin-4-yl)amino)-6-fluorohexan-1-ol 74F (80 mg, 0.18 mmol, 1 equiv.) was treated with DCM (2 mL) and TFA (0.5 mL). After 3 h the reaction mixture was concentrated under reduced pressure and subjected to reverse phase HPLC (10% to 70% MeCN in water with 0.1% TFA using a Hydro-RP column) to furnish, after collection of product fractions and removal of volatiles in vacuo, 74 as its TFA salt. LCMS (m/z): 280.15 [M+H]⁺; t_{R}=0.56 min. on LC/MS Method A. ¹H NMR (400 MHz, Methanol-d₄) δ 8.64 (dd, J=4.3, 1.4 Hz, 1H), 7.84 (dd, J=8.5, 1.4 Hz, 1H), 4.63-4.50 (m, 1H), 4.47 (t, J=6.0 Hz, 1H), 4.35 (t, J=6.0 Hz, 1H), 3.74 (d, J=5.3 Hz, 2H), 1.89-1.61 (m, 4H), 1.60-1.39 (m, 2H). ¹⁹F NMR (377 MHz, Methanol-d₄) δ −77.66, −220.85 (ddd, J=47.6, 25.5, 22.1 Hz).

### Example 75

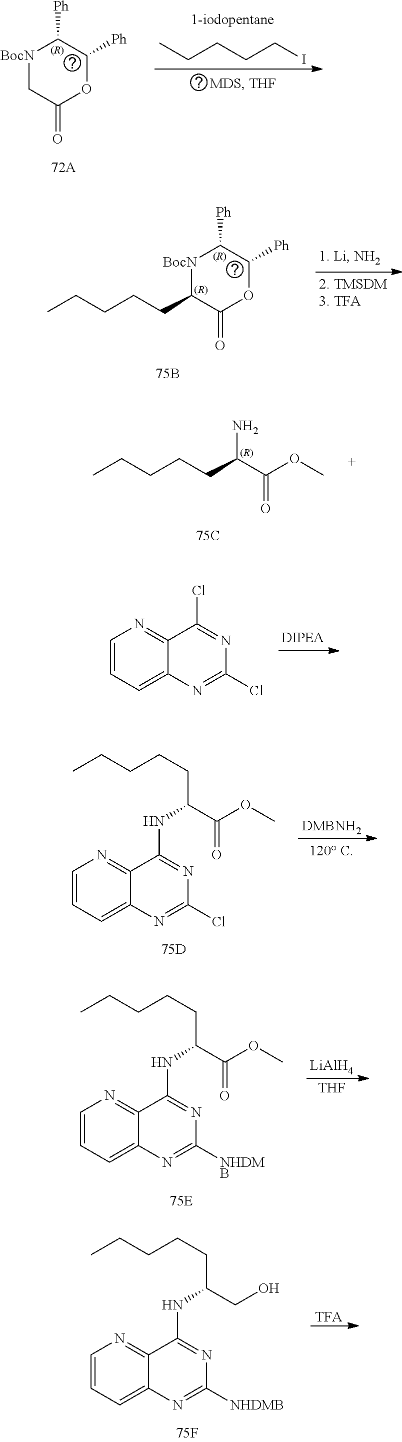

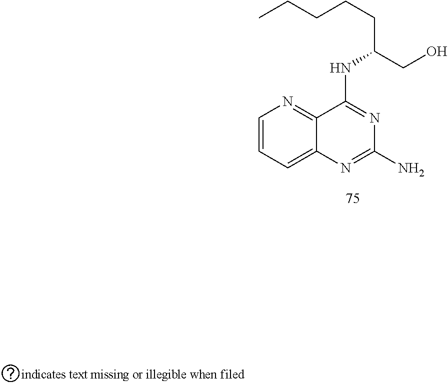 [SMILES: CCCCC[C@H](CO)Nc1nc(N)nc2cccnc12]

Synthesis of (3R,5R,6S)-tert-butyl 2-oxo-3-pentyl-5,6-diphenylmorpholine-4-carboxylate (75B). A stirred solution of (2S,3R)-tert-butyl 6-oxo-2,3-diphenylmorpholine-4-carboxylate 72A (1000 mg, 2.8 mmol, 1 equiv., supplied by Sigma-Aldrich) and 1-iodopentane (1.8 mL, 14.2 mmol, 5 equiv., supplied by Sigma-Aldrich) in anhydrous THF (15 mL) and HMPA (1.5 mL) cooled to −78° C., was treated dropwise with 1M lithium bis(trimethylsilyl) amide in THF (4.2 ml, 1.5 equiv.) under argon. After 10 min. the reaction mixture was stirred at −40° C. for 4 h. The reaction mixture was quenched with EtOAc and poured into a mixture of EtOAc (50 mL) and an aqueous solution of NH₄Cl (50 mL, 1 M). The organic layer was separated and concentrated in vacuo to provide a crude residue which was subjected to silica gel chromatography eluting with 0% to 100% EtOAc in hexanes to afford 75B. LCMS (m/z): 310.08 [M+H]⁺; t_{R}=0.1.33 min. on LC/MS Method A.

Synthesis of (R)-methyl 2-aminoheptanoate (75C). A 2-neck flask containing lithium (110 mg, 15.9 mmol, 15 equiv.) was cooled at −40° C. before liquid ammonia (15 mL) was added via a cold-finger. To the deep blue mixture was added (3R,5R,6S)-tert-butyl 2-oxo-3-pentyl-5,6-diphenylmorpholine-4-carboxylate 75B (450 mg, 1.06 mmol, 1 equiv.). The reaction was maintained at this temperature for 1h and then allowed to warm to rt. The reaction was slowly quenched with NH₄Cl (5 mL) solution and diluted with ether (50 mL) and separated. To the aqueous layer was added 1N HCl to pH 5 which was then extracted with EtOAc (50 mL). Each of the organic layers was then washed separately with saturated NH₄Cl, then combined, dried over MgSO₄, filtered, and concentrated under reduced pressure. The residue was treated with DCM (10 mL) and MeOH (1 mL) along with (trimethylsilyl)diazomethane, 2.0M solution in hexanes (1.1 mL, 2.1 mmol, 4 equiv.). After 1 h the reaction was concentrated under reduced pressure and the residue dissolved in DCM (5 mL) and TFA (5 mL). The mixture was stirred for 2 h and then concentrated under reduced pressure to afford crude 75C which was used without further purification.

Synthesis of (R)-methyl 2-((2-chloropyrido[3,2-d]pyrimidin-4-yl)amino)heptanoate (75D). A solution of 2,4-dichloropyrido[3,2-d]pyrimidine (89 mg, 0.44 mmol, 1.2 equiv.) in THF (5 mL) was treated with N,N-diisopropylethylamine (0.26 mL, 1.76 mmol, 4 equiv.) and (R)-methyl 2-aminoheptanoate 75C (71 mg, 0.44 mmol, 1 equiv., TFA salt). The reaction was stirred for 1 h and then the mixture containing 75D was used without purification. LCMS (m/z): 323.8 [M+H]⁺; t_{R}=1.32 min. on LC/MS Method A.

Synthesis of (R)-methyl 2-((2-((2,4-dimethoxybenzyl)amino)pyrido[3,2-d]pyrimidin-4-yl)amino)heptanoate (75E). To the solution containing (R)-methyl 2-((2-chloropyrido[3,2-d]pyrimidin-4-yl)amino)heptanoate 75D (120 mg, 0.37 mmol, 1 equiv.) prepared as described, was added 2,4-dimethoxybenzylamine (0.17 mL, 1.1 mmol, 3 equiv.). The reaction mixture was heated at 120° C. overnight. The reaction mixture partitioned between EtOAc (50 mL) and H₂O (50 mL). The organic layer was separated, dried, and concentrated in vacuo. The residue was subjected to silica gel chromatography eluting with 0% to 100% EtOAc in hexanes to provide the title compound 75E. LCMS (m/z): 454.6 [M+H]⁺; t_{R}=1.02 min. on LC/MS Method A.

Synthesis of (R)-2-((2-((2,4-dimethoxybenzyl)amino)pyrido[3,2-d]pyrimidin-4-yl)amino)heptan-1-ol (75F). (R)-methyl 2-((2-((2,4-dimethoxybenzyl)amino)pyrido[3,2-d]pyrimidin-4-yl)amino)heptanoate 75E (169 mg, 0.37 mmol, 1 equiv.) was dissolved in THF (5 mL) and treated with 1M lithium aluminum hydride in ether (1.1 mL, 1.1 mmol, 3 equiv.). The reaction mixture was stirred at rt. After 2 h, the reaction was quenched with water and extracted with EtOAc. The organics were separated, dried, and concentrated in vacuo. The crude product 75F was used without further purification. LCMS (m/z): 426.4 [M+H]⁺; t_{R}=0.95 min. on LC/MS Method A.

Synthesis of (R)-2-((2-aminopyrido[3,2-d]pyrimidin-4-yl)amino)heptan-1-ol (75). (R)-2-((2-((2,4-dimethoxybenzyl)amino)pyrido[3,2-d]pyrimidin-4-yl)amino)heptan-1-ol 75F (20 mg, 0.05 mmol, 1 equiv.) was dissolved in DCM (2 mL) and TFA (0.5 mL). After 3 h the reaction mixture was concentrated under reduced pressure and the residue subjected to reverse phase HPLC (10% to 70% MeCN in water with 0.1% TFA using a Hydro-RP column) to furnish, after collection of product fractions and removal of volatiles in vacuo, 75 as its TFA salt. LCMS (m/z): 276.4 [M+H]⁺; t_{R}=0.71 min. on LC/MS Method A. ¹H NMR (400 MHz, Methanol-d₄) δ 8.65 (dd, J=4.3, 1.6 Hz, 1H), 7.92-7.66 (m, 2H), 4.66-4.43 (m, 1H), 3.73 (d, J=5.3 Hz, 2H), 1.81-1.57 (m, 2H), 1.51-1.20 (m, 9H), 0.89 (t, J=7.0 Hz, 3H). ¹⁹F NMR (377 MHz, Methanol-d₄) δ −77.55.

### Example 76 and Example 77

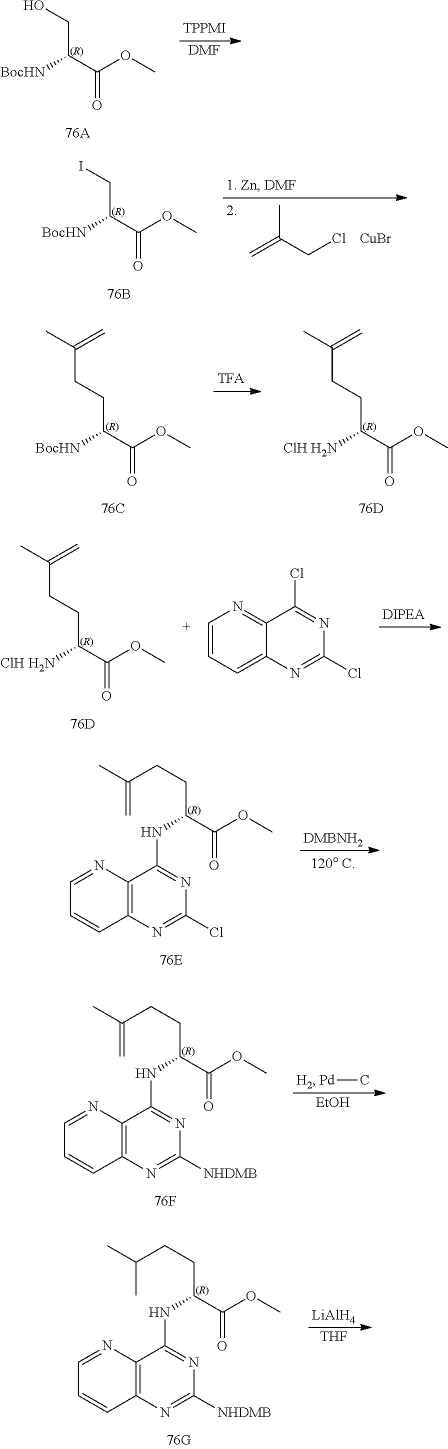

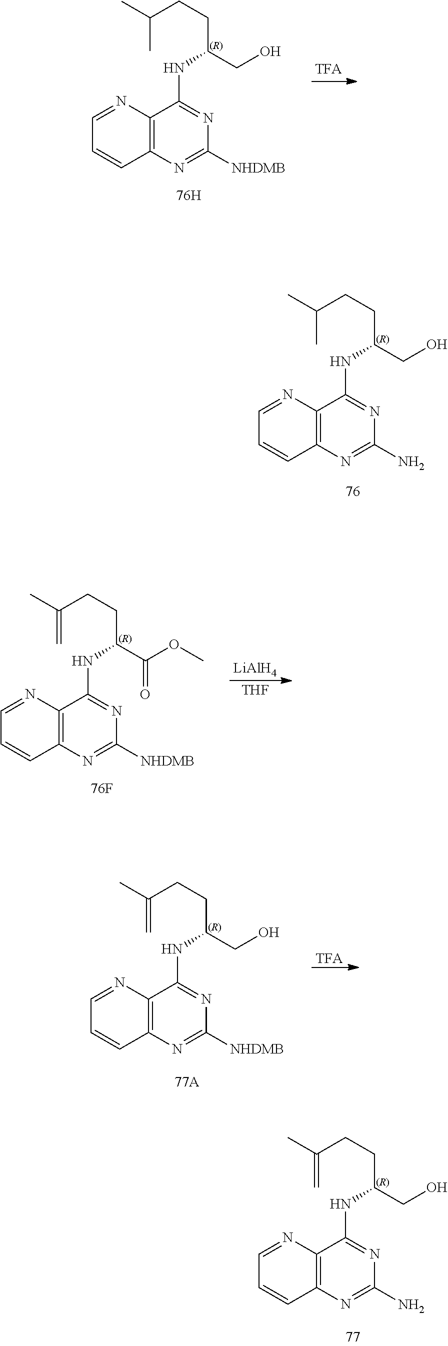

Synthesis of (S)-methyl 2-((tert-butoxycarbonyl)amino)-3-iodopropanoate (76B). (R)-methyl 2-((tert-butoxycarbonyl)amino)-3-hydroxypropanoate 76A (6 g, 27.37 mmol, supplied by Sigma-Aldrich) was treated with DMF (100 mL) and cooled to 0° C. before methyltriphenoxyphosphonium iodide (16.1 g, 35.58 mmol, 1.3 equiv., supplied by Sigma-Aldrich) was slowly added. The reaction mixture was stirred overnight and solid NaHCO₃ (14 g) and water (100 mL) were added to the reaction. The reaction mixture was stirred for 15 min. and then the mixture was extracted with hexanes in diethyl ether, (1:1) (2×250 mL). The combined organic extracts were washed with 0.5M NaOH solution (3×75 mL) and saturated NH₄Cl (75 mL), dried over MgSO₄, filtered and concentrated under reduced pressure to afford the crude product 76B. LCMS (m/z): 331.13 [M+H]⁺; t_{R}=1.16 min. on LC/MS Method A.

Synthesis of (R)-methyl 2-((tert-butoxycarbonyl)amino)-5-methylhex-5-enoate (76C). Zinc dust (2.4 g, 36.4 mmol, 4 equiv.) was added to iodine (93 mg, 0.37 mmol, 0.04 equiv.) in a three-neck round-bottomed flask and heated under vacuum for 10 min. The flask was flushed with nitrogen and evacuated three times. (S)-methyl 2-((tert-butoxycarbonyl)amino)-3-iodopropanoate 76B (3000 mg, 9.11 mmol) was dissolved in dry DMF (5 mL) and added to the zinc slurry at 0° C. The reaction mixture was stirred at rt for 1 h. Copper (I) bromide-dimethylsulfide complex (187.39 mg, 0.91 mmol, 0.1 equiv., supplied by Sigma-Aldrich) was placed in a separate three-necked flask and gently dried under vacuum until a color change from white to green was observed. Dry DMF (4 mL) and 3-chloro-2-methylpropene (1.34 mL, 13.67 mmol, supplied by Sigma-Aldrich) were added, and the reaction was cooled to −15° C. Once zinc insertion in the first step was complete, stirring was stopped, and the zinc allowed to settle. The supernatant was removed via syringe and added dropwise to the electrophile and Cu catalyst mixture at −15° C. The cold bath was removed, and the reaction mixture was stirred at rt for 2 days. EtOAc (100 mL) was added, and the reaction was stirred for 15 min. The reaction mixture was washed with 1M Na₂S₂O₃ (100 mL), water (2×100 mL), and brine (100 mL), dried over MgSO₄, filtered, and concentrated reduced pressure. The residue was subjected to silica gel chromatography eluting with 0% to 100% EtOAc in hexane to provide 76C. LCMS (m/z): 157.95 [M+H-Boc]⁺; t_{R}=1.16 min. on LC/MS Method A.

Synthesis of (R)-methyl 2-amino-5-methylhex-5-enoate (76D). (R)-methyl 2-((tert-butoxycarbonyl)amino)heptanoate 76C (655 mg, 3 mmol) was treated with DCM (5 mL) and TFA (5 mL) and stirred for 2 h. The mixture was then concentrated under reduced pressure to provide 76D that was used without further purification.

Synthesis of (R)-methyl 2-((2-chloropyrido[3,2-d]pyrimidin-4-yl)amino)-5-methylhex-5-enoate (76E). 2,4-dichloropyrido[3,2-d]pyrimidine (466 mg, 2 mmol, 1 equiv.) was treated with THF (10 mL) followed by N,N-diisopropylethylamine (1.66 mL, 9 mmol, 4 equiv.), and then (R)-methyl 2-amino-5-methylhex-5-enoate 76D (593 mg, 2 mmol, 1 equiv., TFA salt). The reaction mixture was stirred for 1 h and then the product 76E was used directly. LCMS (m/z): 321.2 [M+H]⁺; t_{R}=1.19 min. on LC/MS Method A.

Synthesis of (R)-methyl 2-((2-((2,4-dimethoxybenzyl)amino)pyrido[3,2-d]pyrimidin-4-yl)amino)-5-methylhex-5-enoate (76F). The solution of (R)-methyl 2-((2-chloropyrido[3,2-d]pyrimidin-4-yl)amino)-5-methylhex-5-enoate 76E (748 mg, 2 mmol, 1 equiv.) prepared as described, was treated with 2,4-dimethoxybenzylamine (0.69 mL, 5 mmol, 2 equiv.) and N,N-diisopropylethylamine (1.66 mL, 9 mmol, 4 equiv.). The reaction mixture was heated at 120° C. overnight. The reaction mixture was partitioned between EtOAc (50 mL) and H₂O (50 mL). The organic layer was separated, dried over MgSO₄, and concentrated in vacuo. The residue was subjected to silica gel chromatography eluting with 0% to 100% EtOAc in hexane to provide the title compound 76F (LCMS (m/z): 452.55 [M+H]⁺; t_{R}=0.97 min. on LC/MS Method A.

Synthesis of (R)-methyl 2-((2-((2,4-dimethoxybenzyl)amino)pyrido[3,2-d]pyrimidin-4-yl)amino)-5-methylhexanoate (76G). (R)-methyl 2-((2-((2,4-dimethoxybenzyl)amino)pyrido[3,2-d]pyrimidin-4-yl)amino)-5-methylhex-5-enoate 76F (35 mg, 0.08 mmol) was treated with Pd/C (50 mg) and EtOH (5 mL) and then stirred under hydrogen. After 4 h the solid was removed by filtration and the filtrate was concentrated under reduced pressure. The resulting residue of 76G was used without further purification. LCMS (m/z): 454.24 [M+H]⁺; t_{R}=1.06 min. on LC/MS Method A.

Synthesis of (R)-2-((2-((2,4-dimethoxybenzyl)amino)pyrido[3,2-d]pyrimidin-4-yl)amino)-5-methylhexan-1-ol (76H). (R)-methyl 2-((2-((2,4-dimethoxybenzyl)amino)pyrido[3,2-d]pyrimidin-4-yl)amino)-5-methylhexanoate 76G (32 mg, 0.37 mmol, 1 equiv.) was treated with THF (5 mL) and 1M lithium aluminum hydride in ether (0.2 mL, 0.2 mmol, 3 equiv.). The reaction mixture was stirred for 2 h and then quenched with water (50 mL) and extracted with EtOAc (50 mL). The organic layer was separated, dried over MgSO₄, and concentrated in vacuo. The crude material 76H was used without further purification. LCMS (m/z): 426.23 [M+H]⁺; t_{R}=0.96 min. on LC/MS Method A.

Synthesis of (R)-2-((2-aminopyrido[3,2-d]pyrimidin-4-yl)amino)-5-methylhexan-1-ol. (R)-2-((2-((2,4-dimethoxybenzyl)amino)pyrido[3,2-d]pyrimidin-4-yl)amino)-5-methylhexan-1-ol (76). Compound 76H (25 mg, 0.05 mmol, 1 equiv.) was treated with DCM (2 mL) and TFA (0.5 mL). After 3 h the reaction mixture was concentrated under reduced pressure and subjected to reverse phase HPLC (10% to 70% MeCN in water with 0.1% TFA using a Hydro-RP) to furnish, after collection of product fractions and removal of volatiles in vacuo, 76. LCMS (m/z): 276.13 [M+H]⁺; t_{R}=0.70 min. on LC/MS Method A.

Synthesis of (R)-2-((2-((2,4-dimethoxybenzyl)amino)pyrido[3,2-d]pyrimidin-4-yl)amino)-5-methylhex-5-en-1-ol (77A). (R)-methyl 2-((2-((2,4-dimethoxybenzyl)amino)pyrido[3,2-d]pyrimidin-4-yl)amino)-5-methylhex-5-enoate 76F (40 mg, 90 mmol, 1 equiv.) was treated with THF (5 mL) and 1M lithium aluminum hydride in ether (0.27 mL, 0.27 mmol, 3 equiv.). The reaction mixture was stirred for 2 h and then quenched with water (50 ml) and extracted with EtOAc (50 mL). The organics were separated, dried, and concentrated in vacuo to provide a residue of 77A that was used without further purification. LCMS (m/z): 424.20 [M+H]⁺; t_{R}=0.88 min. on LC/MS Method A.

Synthesis of (R)-2-((2-aminopyrido[3,2-d]pyrimidin-4-yl)amino)-5-methylhex-5-en-1-ol (77). 77A (40 mg, 0.095 mmol, 1 equiv.) was treated with DCM (2 mL) and TFA (0.5 mL). After 3 h the reaction mixture was concentrated under reduced pressure and subjected to reverse phase HPLC (10% to 70% MeCN in water with 0.1% TFA using a Hydro-RP column) to furnish, after collection of product fractions and removal of volatiles in vacuo, the title compound 77 as its TFA salt. LCMS (m/z): 274.43 [M+H]⁺; t_{R}=0.65 min. on LC/MS Method A. ¹H NMR (400 MHz, Methanol-d₄) δ 8.59-8.42 (m, 1H), 7.75-7.52 (m, 2H), 4.45-4.13 (m, 1H), 3.87-3.69 (m, 1H), 3.65-3.44 (m, 2H), 2.30 (dq, J=15.0, 7.1 Hz, 1H), 2.01-1.73 (m, 2H), 1.68-1.41 (m, 4H), 1.26-1.05 (m, 6H). ¹⁹F NMR (377 MHz, Methanol-d₄) δ −77.52.

### Example 78

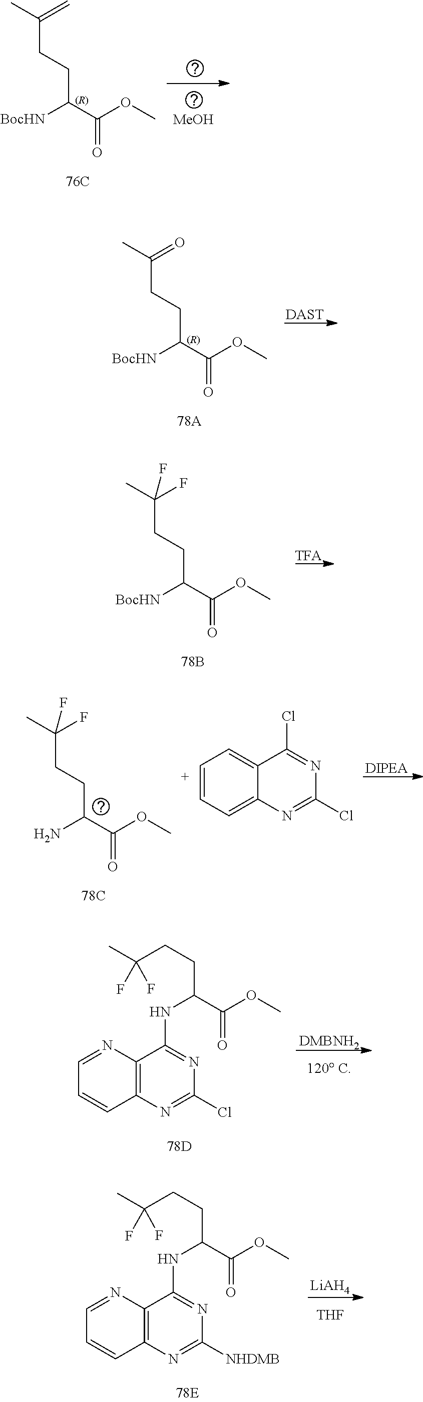

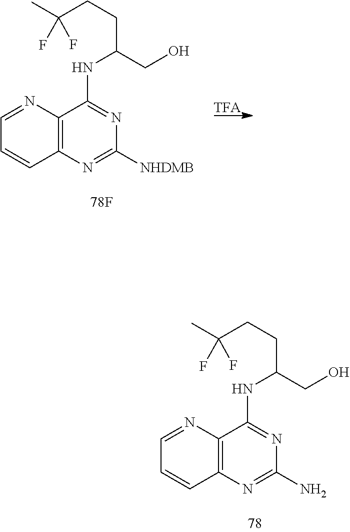

Synthesis of (R)-methyl 2-((tert-butoxycarbonyl)amino)-5-oxohexanoate (78A). (R)-methyl 2-((tert-butoxycarbonyl)amino)-5-methylhex-5-enoate 76C (775 mg, 3.01 mmol) was treated with DCM (20 mL) and MeOH (5 mL) before cooling to −78° C. Ozone was bubbled through the reaction mixture. After 10 min., the mixture was quenched with dimethyl sulfide (0.90 mL, 12 mmol, 4 equiv.) and allowed to warm up to rt. EtOAc (100 mL) was added, and the reaction was stirred for 15 min. The mixture was washed with 1M Na₂S₂O₃ (100 mL), water (2×100 mL), and brine (100 mL) and dried over MgSO₄. The organic solution was filtered and concentrated under reduced pressure, and the resulting residue was subjected to silica gel chromatography eluting with 0% to 100% EtOAc in hexane to provide 78A ¹H NMR (400 MHz, Chloroform-d) δ 5.11 (d, J=8.3 Hz, 1H), 4.33-4.20 (m, 1H), 3.73 (s, 4H), 2.63-2.42 (m, 3H), 2.14 (s, 4H), 2.12-2.05 (m, 1H), 1.94-1.81 (m, 1H), 1.42 (s, 13H).

Synthesis of (R)-methyl 2-((tert-butoxycarbonyl)amino)-5,5-difluorohexanoate (78B). (R)-methyl 2-((tert-butoxycarbonyl)amino)-5-oxohexanoate 78A (235 mg, 0.91 mmol) was dissolved in DCM (10 mL), then treated with DAST 95% (0.36 mL, 2.72 mmol). The reaction was stirred for 16 h. EtOAc (50 mL) and NaHCO₃ solution (5 mL) were added and the reaction was stirred for 5 min. The reaction mixture was washed with 1M Na₂S₂O₃ (100 mL), water (2×100 mL), and brine (100 mL) and dried over MgSO₄. The solvent was removed under reduced pressure and the residue subjected to silica gel chromatography eluting with 0% to 100% EtOAc in hexanes to afford 78B. ¹H NMR (400 MHz, Chloroform-d) δ 5.04 (s, 1H), 4.32 (s, 1H), 3.76 (s, 5H), 2.16-1.99 (m, 2H), 1.98-1.75 (m, 5H), 1.69-1.52 (m, 7H), 1.44 (s, 16H), 1.34-1.20 (m, 2H), 0.92-0.80 (m, 1H). ¹⁹F NMR (377 MHz, Chloroform-d) δ −92.14 (dq, J=50.1, 17.0 Hz).

Synthesis of (R)-methyl 2-amino-5,5-difluorohexanoate (78C). (R)-methyl 2-((tert-butoxycarbonyl)amino)-5,5-difluorohexanoate 78B (36 mg, 0.13 mmol, 1 equiv.) was treated with DCM (2 mL) and TFA (0.5 mL). After 3 h the reaction mixture was concentrated under reduced pressure and the crude product 78C was used without further purification.

Synthesis of (R)-methyl 2-((2-chloropyrido[3,2-d]pyrimidin-4-yl)amino)-5,5-difluorohexanoate (78D). 2,4-dichloropyrido[3,2-d]pyrimidine (33 mg, 0.16 mmol, 1.25 equiv.) was treated with THF (10 mL) followed by N,N-diisopropylethylamine (0.18 mL, 1.0 mmol, 8 equiv.), and (R)-methyl 2-amino-5,5-difluorohexanoate 78C (36 mg, 0.13 mmol, 1 equiv., TFA salt). The reaction mixture was stirred for 1 h to generate 78D and then this mixture was used directly. LCMS (m/z): 345.13 [M+H]⁺; t_{R}=1.08 min. on LC/MS Method A.

Synthesis of (R)-methyl 2-((2-((2,4-dimethoxybenzyl)amino)pyrido[3,2-d]pyrimidin-4-yl)amino)-5,5-difluorohexanoate (78E). (R)-methyl 2-((2-chloropyrido[3,2-d]pyrimidin-4-yl)amino)-5,5-difluorohexanoate 78D (45 mg, 0.13 mmol, 1 equiv.) solution as described, was treated with 2,4-dimethoxybenzylamine (0.077 mL, 0.52 mmol, 4 equiv.). The reaction was heated at 120° C. overnight. The reaction mixture was partitioned between EtOAc (100 mL) and H₂O (100 mL). The organics were separated, dried, and concentrated in vacuo. The residue was subjected to silica gel chromatography eluting with 0% to 100% EtOAc in hexane to provide the title compound 78E. LCMS (m/z): 476.13 [M+H]⁺; t_{R}=0.99 min. on LC/MS Method A.

Synthesis of (R)-2-((2-((2,4-dimethoxybenzyl)amino)pyrido[3,2-d]pyrimidin-4-yl)amino)-5,5-difluorohexan-1-ol (78F). (R)-methyl 2-((2-((2,4-dimethoxybenzyl)amino)pyrido[3,2-d]pyrimidin-4-yl)amino)-5,5-difluorohexanoate 78E (26 mg, 0.055 mmol, 1 equiv.) was treated with THF (5 mL) and 1M lithium aluminum hydride in ether (0.2 mL, 0.2 mmol, 4 equiv.). The reaction mixture was stirred at rt for 2 h and then the reaction was quenched with water (50 mL) and extracted with EtOAc (50 mL). The organics were separated, dried, and concentrated in vacuo. The crude material 78E was used without further purification. LCMS (m/z): 448.12 [M+H]⁺; t_{R}=0.91 min. on LC/MS Method A.

Synthesis of (R)-2-((2-aminopyrido[3,2-d]pyrimidin-4-yl)amino)-5,5-difluorohexan-1-ol (78). (R)-2-((2-((2,4-dimethoxybenzyl)amino)pyrido[3,2-d]pyrimidin-4-yl)amino)-5,5-difluorohexan-1-ol 78F (24 mg, 0.055 mmol, 1 equiv.) was treated with DCM (2 mL) and TFA (0.5 mL). After 3 h the reaction mixture was concentrated under reduced pressure and subjected to reverse phase HPLC (10% to 70% MeCN in water with 0.1% TFA using a Hydro-RP column) to furnish, after collection of product fractions and removal of volatiles in vacuo, 78. LCMS (m/z): 298.10 [M+H]⁺; t_{R}=0.60 min. on LC/MS Method A. ¹H NMR (400 MHz, Methanol-d₄) δ 8.66 (dd, J=4.3, 1.5 Hz, 5H), 7.86-7.73 (m, 10H), 4.55 (dd, J=9.0, 4.7 Hz, 5H), 4.30 (s, 1H), 3.83 (s, 2H), 3.76 (t, J=5.1 Hz, 12H), 3.34 (s, 3H), 2.05-1.85 (m, 23H), 1.58 (t, J=18.5 Hz, 17H), 1.41-1.26 (m, 17H), 1.14 (s, 1H), 0.96-0.88 (m, 4H), 0.87 (s, 2H). ¹⁹F NMR (377 MHz, Methanol-d₄) δ −77.67, −92.96 (p, J=17.4 Hz).

### Example 79

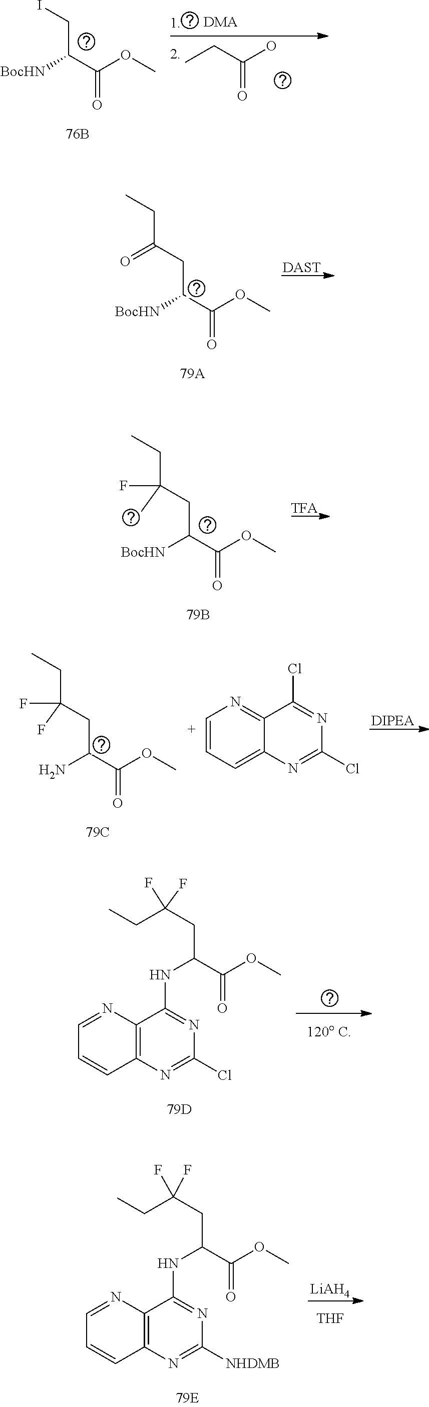

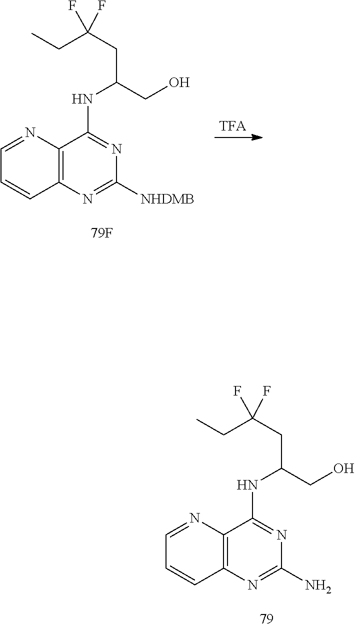

Synthesis of (R)-methyl 2-((tert-butoxycarbonyl)amino)-4-oxohexanoate (79A). Zinc dust (1.58 g, 24.3 mmol, 4 equiv.) was added to iodine (61 mg, 0.24 mmol, 0.04 equiv.) in a three-neck round-bottomed flask and heated under vacuum for 10 min. The flask was flushed with nitrogen and evacuated three times. After cooling, benzene (10 mL) and DMA (1 mL) were added. 1,2-bromoethane (0.05 mL, 0.61 mmol) and chlorotrimethylsilane (33.01 mg, 0.3 mmol) were then added consecutively and this process repeated three times in the course of 1 hour. (S)-methyl 2-((tert-butoxycarbonyl)amino)-3-iodopropanoate 76B (2400 mg, 0.6 mmol, 1 equiv.) was dissolved in benzene (10 mL) and DMA (1 mL) and added to the zinc slurry. After about 1 h, bis(triphenylphosphine) palladium (II) dichloride, (106.62 mg, 0.025 equiv.) and Tetrakis(triphenylphosphine)palladium(0) (175.68 mg, 0.025 equiv.) were added followed by propionyl chloride (0.8 mL, 0.01 mol, 1.5 equiv.). The reaction mixture was warmed to 70° C. and stirred for 1 h. EtOAc (100 mL) was added, and the reaction mixture was filtered over a pad of Celite. The filtrate was washed with water (2×100 mL), brine (100 mL), dried over MgSO₄, filtered and concentrated under reduced pressure. The residue was subjected to silica gel chromatography eluting with 0% to 100% EtOAc in hexane to afford 79A. ¹H NMR (400 MHz, Chloroform-d) δ 5.48 (d, J=8.6 Hz, 1H), 4.46 (dt, J=8.7, 4.4 Hz, 1H), 3.69 (s, 3H), 3.10 (dd, J=18.0, 4.5 Hz, 1H), 2.89 (dd, J=17.9, 4.4 Hz, 1H), 2.40 (qd, J=7.3, 1.7 Hz, 2H), 1.40 (s, 10H), 1.01 (t, J=7.3 Hz, 3H).

Synthesis of (R)-methyl 2-((tert-butoxycarbonyl)amino)-4,4-difluorohexanoate (79B). (R)-methyl 2-((tert-butoxycarbonyl)amino)-4-oxohexanoate 79A (475 mg, 1.8 mmol, 1 equiv.) was treated with DAST (0.97 mL, 7.3 mmol, 4 equiv.). The reaction mixture was stirred for 16 h. EtOAc (50 mL) and NaHCO₃ solution (5 mL) were added and the reaction was stirred for 5 min. The reaction mixture was washed with 1M Na₂S₂O₃ (100 mL), water (2×100 mL), brine (100 mL), dried over MgSO₄, filtered, and concentrated under reduced pressure. The residue was subjected to silica gel chromatography eluting with 0% to 100% EtOAc in hexane to afford 79B. ¹H NMR (400 MHz, Chloroform-d) δ 5.20 (d, J=8.3 Hz, 1H), 4.51 (d, J=7.0 Hz, 1H), 3.82 (s, 1H), 3.75 (d, J=0.5 Hz, 5H), 3.35-3.17 (m, 2H), 3.11 (q, J=7.1 Hz, 2H), 2.52-2.27 (m, 3H), 1.89 (ddt, J=24.1, 16.8, 7.5 Hz, 3H), 1.44 (d, J=0.6 Hz, 15H), 1.23-1.13 (m, 4H), 1.00 (dt, J=10.7, 7.5 Hz, 6H). ¹⁹F NMR (377 MHz, Chloroform-d) δ −93.56-−109.28 (m).

Synthesis of (R)-methyl 2-amino-5,5-difluorohexanoate. (R)-methyl 2-((tert-butoxycarbonyl)amino)-4,4-difluorohexanoate (79C). Compound 79B (98 mg, 0.35 mmol, 1 equiv.) was treated with DCM (2 mL) and TFA (0.5 mL). After 3 h the reaction mixture was concentrated under reduced pressure and the crude product 79C as its TFA salt was used without further purification.

Synthesis of (R)-methyl 2-((2-chloropyrido[3,2-d]pyrimidin-4-yl)amino)-4,4-difluorohexanoate (79D). 2,4-dichloropyrido[3,2-d]pyrimidine (80 mg, 0.39 mmol, 1 equiv.) was treated with THF (10 ml) followed by N,N-diisopropylethylamine (0.28 mL, 1.5 mmol, 4 equiv.), and then (R)-methyl 2-amino-5,5-difluorohexanoate 79C (110 mg, 0.39 mmol, 1 equiv., TFA salt). The reaction mixture was stirred for 1 h to form 79D and then this solution was used directly. LCMS (m/z): 345.11 [M+H]⁺; t_{R}=1.09 min. on LC/MS Method A.

Synthesis of (R)-methyl 2-((2-((2,4-dimethoxybenzyl)amino)pyrido[3,2-d]pyrimidin-4-yl)amino)-4,4-difluorohexanoate (79E). (R)-methyl 2-((2-chloropyrido[3,2-d]pyrimidin-4-yl)amino)-5,5-difluorohexanoate 79D solution prepared as described, was treated with 2,4-dimethoxybenzylamine (0.077 mL, 0.52 mmol, 4 equiv.). The reaction was heated at 120° C. overnight. The reaction mixture partitioned between EtOAc (50 mL) and H₂O (50 mL). The organics were separated, dried over MgSO₄, and concentrated in vacuo. The residue was subjected to silica gel chromatography eluting with 0% to 100% EtOAc in hexane to provide 79E. LCMS (m/z): 476.32 [M+H]⁺; t_{R}=0.96 min. on LC/MS Method A.

Synthesis of (R)-2-((2-((2,4-dimethoxybenzyl)amino)pyrido[3,2-d]pyrimidin-4-yl)amino)-4,4-difluorohexan-1-ol (79F). (R)-methyl 2-((2-((2,4-dimethoxybenzyl)amino)pyrido[3,2-d]pyrimidin-4-yl)amino)-4,4-difluorohexanoate 79E (35 mg, 0.074 mmol, 1 equiv.) was treated with THF (5 mL) and 1M lithium aluminum hydride in ether (0.29 mL, 0.29 mmol, 4 equiv.). The reaction mixture was stirred for 2 h and then the reaction was quenched with water (50 mL) and extracted with EtOAc (50 mL). The organic layer was separated, dried over MgSO₄, and concentrated in vacuo. The crude material 79F was used without further purification. LCMS (m/z): 448.20 [M+H]⁺; t_{R}=0.86 min. on LC/MS Method A.

Synthesis of (R)-2-((2-aminopyrido[3,2-d]pyrimidin-4-yl)amino)-4,4-difluorohexan-1-ol (79). (R)-2-((2-((2,4-dimethoxybenzyl)amino)pyrido[3,2-d]pyrimidin-4-yl)amino)-4,4-difluorohexan-1-ol 79F (24 mg, 0.055 mmol, 1 equiv.) was treated with DCM (2 mL) and TFA (0.5 mL). After 3 h the reaction mixture was concentrated under reduced pressure and subjected to reverse phase HPLC (10% to 70% MeCN in water with 0.1% TFA using a Hydro-RP column) to furnish, after collection of product fractions and removal of volatiles in vacuo, 79 as its TFA salt. LCMS (m/z): 298.11 [M+H]⁺; t_{R}=0.63 min. on LC/MS Method A. ¹H NMR (400 MHz, Methanol-d₄) δ 8.51 (dd, J=4.3, 1.5 Hz, 1H), 7.77-7.54 (m, 2H), 3.60 (d, J=5.7 Hz, 2H), 2.37-2.11 (m, 2H), 1.93-1.69 (m, 2H), 0.87 (t, J=7.5 Hz, 3H). ¹⁹F NMR (377 MHz, Methanol-d₄) δ −77.80, −98.15, −105.45 (m).

### Example 80 and Example 81

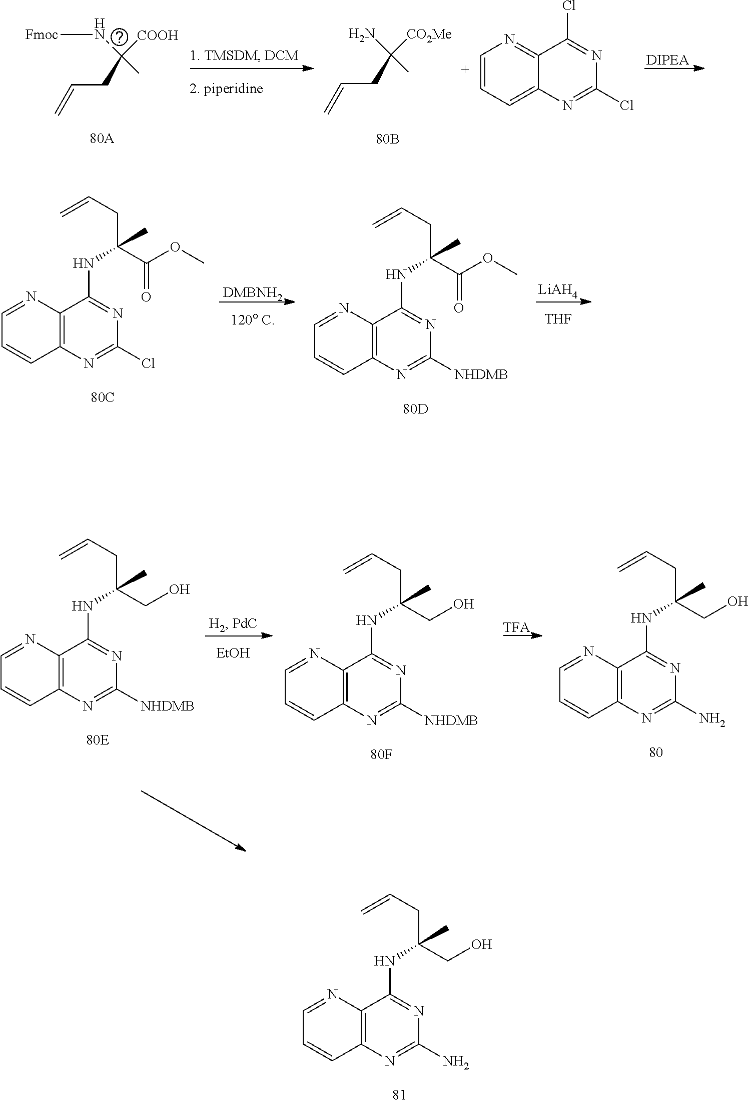

Synthesis of (R)-methyl 2-amino-2-methylpent-4-enoate (80B). (R)-2-((((9H-fluoren-9-yl)methoxy)carbonyl)amino)-2-methylpent-4-enoic acid 80 A (1 g, 2.8 mmol, 1 equiv., provided by Okeanos Inc.) was treated with DCM (10 mL) and MeOH (1 mL) along with (trimethylsilyl)diazomethane (2.0M solution in hexanes, 2.3 mL, 5.6 mmol, 2.5 equiv.). After 1 h the reaction mixture was concentrated under reduced pressure to provide a residue. The residue was treated with THF (10 mL) followed by piperidine (0.56 mL, 0.006 mol, 2 equiv.). The mixture was stirred for 2 h and then concentrated under reduced pressure to provide 80B that was used without further purification.

Synthesis of (R)-methyl 2-((2-chloropyrido[3,2-d]pyrimidin-4-yl)amino)-2-methylpent-4-enoate (80C). 2,4-dichloropyrido[3,2-d]pyrimidine (540 mg, 2.71 mmol, 1 equiv.) was treated with dioxane (15 ml) followed by N,N-diisopropylethylamine (1.9 mL, 10.8 mmol, 4 equiv.), and then (R)-methyl 2-amino-2-methylpent-4-enoate 80B (486 mg, 2.71 mmol, 1 equiv.). The reaction mixture was stirred at 80° C. for 15 minutes, then more 2,4-dichloropyrido[3,2-d]pyrimidine (250 mg, 1.25 mmol) was added. The mixture was stirred at 80° C. overnight to form 80C which was then used directly. LCMS (m/z): 307.12 [M+H]⁺; t_{R}=1.14 min. on LC/MS Method A.

Synthesis of (R)-methyl 2-((2-((2,4-dimethoxybenzyl)amino)pyrido[3,2-d]pyrimidin-4-yl)amino)-2-methylpent-4-enoate (80D). (R)-methyl 2-((2-chloropyrido[3,2-d]pyrimidin-4-yl)amino)-2-methylpent-4-enoate 80C solution prepared as described was treated with 2,4-dimethoxybenzylamine (0.80 mL, 5.0 mmol, 2 equiv.). The reaction was heated at 120° C. overnight. The reaction mixture was partitioned between EtOAc (50 mL) and H₂O (50 mL). The organics were separated, dried over MgSO₄, and concentrated in vacuo. The residue was subjected to silica gel chromatography eluting with 0% to 100% EtOAc in hexane to provide 80D. LCMS (m/z): 438.20 [M+H]⁺; t_{R}=1.04 min. on LC/MS Method A.

Synthesis of (R)-2-((2-((2,4-dimethoxybenzyl)amino)pyrido[3,2-d]pyrimidin-4-yl)amino)-2-methylpent-4-en-1-ol (80E). (R)-methyl 2-((2-((2,4-dimethoxybenzyl)amino)pyrido[3,2-d]pyrimidin-4-yl)amino)-2-methylpent-4-enoate 80D (634 mg, 1.44 mmol, 1 equiv.) was treated with THF (20 mL) and 1M lithium aluminum hydride in ether (3.6 mL, 3.62 mmol, 2.5 equiv.). The reaction mixture was stirred for 2 h and then the reaction was quenched with water (100 mL) and extracted with EtOAc (100 mL). The organic layer was separated, dried over MgSO₄, and concentrated in vacuo. The residue was subjected to silica gel chromatography eluting with 0% to 100% EtOAc in hexane to provide the 80E. LCMS (m/z): 410.17 [M+H]⁺; t_{R}=0.97 min. on LC/MS Method A.

Synthesis of (R)-2-((2-((2,4-dimethoxybenzyl)amino)pyrido[3,2-d]pyrimidin-4-yl)amino)-2-methylpentan-1-ol (80F). (R)-methyl 2-((2-((2,4-dimethoxybenzyl)amino)pyrido[3,2-d]pyrimidin-4-yl)amino)-5-methylhex-5-enoate 80E (35 mg, 0.09 mmol) was treated with Pd/C (60 mg) and EtOH (5 mL) and then stirred under hydrogen. After 24 h, the solid was filtered off and the filtrate was concentrated under reduced pressure. The resulting residue 80F was used without further purification. LCMS (m/z): 454.24 [M+H]⁺; t_{R}=1.06 min. on LC/MS Method A.

Synthesis of (R)-2-((2-aminopyrido[3,2-d]pyrimidin-4-yl)amino)-2-methylpentan-1-ol (80). (R)-2-((2-((2,4-dimethoxybenzyl)amino)pyrido[3,2-d]pyrimidin-4-yl)amino)-2-methylpentan-1-ol 80F (35 mg, 0.09 mmol, 1 equiv.) was treated with DCM (2 mL) and TFA (0.5 mL). After 3 h the reaction mixture was concentrated under reduced pressure and subjected to reverse phase HPLC (10% to 70% MeCN in water with 0.1% TFA using a Hydro-RP column) to furnish, after collection of product fractions and removal of volatiles in vacuo, 80 as its TFA salt. LCMS (m/z): 262.13 [M+H]⁺; t_{R}=0.64 min. on LC/MS Method A.

Synthesis of (R)-2-((2-aminopyrido[3,2-d]pyrimidin-4-yl)amino)-2-methylpent-4-en-1-ol (81). (R)-methyl 2-((2-((2,4-dimethoxybenzyl)amino)pyrido[3,2-d]pyrimidin-4-yl)amino)-5-methylhex-5-enoate 80E (40 mg, 0.10 mmol, 1 equiv.) was treated with DCM (2 mL) and TFA (0.5 mL). After 4 h the reaction mixture was concentrated under reduced pressure and subjected to reverse phase HPLC (10% to 70% MeCN in water with 0.1% TFA using a Hydro-RP column) to furnish, after collection of product fractions and removal of volatiles in vacuo, 81 as its TFA salt. LCMS (m/z): 260.10 [M+H]⁺; t_{R}=0.63 min. on LC/MS Method A. ¹H NMR (400 MHz, Methanol-d₄) δ 8.59 (dd, J=4.4, 1.4 Hz, 1H), 7.84 (dd, J=8.5, 1.4 Hz, 1H), 7.75 (dd, J=8.5, 4.4 Hz, 1H), 5.87 (ddt, J=17.5, 10.1, 7.4 Hz, 1H), 5.33-4.94 (m, 2H), 3.94 (d, J=11.2 Hz, 1H), 3.78 (d, J=11.2 Hz, 1H), 2.97-2.76 (m, 1H), 2.70 (ddt, J=13.9, 7.3, 1.2 Hz, 1H), 1.55 (s, 3H). ¹⁹F NMR (377 MHz, Methanol-d₄) δ −77.56.

### Example 82

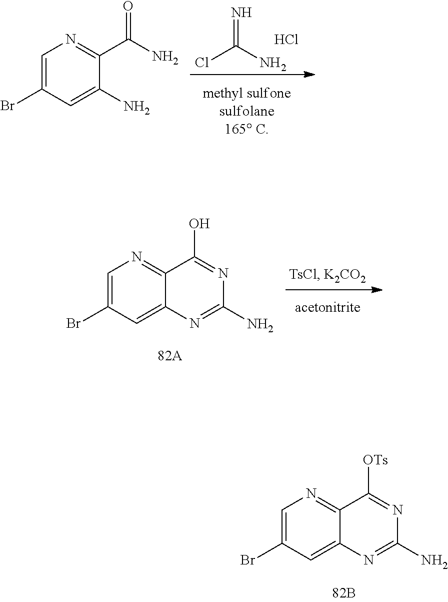

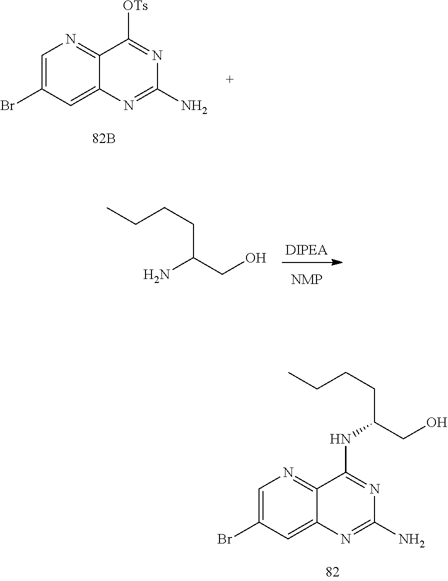

Synthesis of 2-amino-7-bromopyrido[3,2-d]pyrimidin-4-ol (82A). A mixture of 3-amino-5-bromopyridine-2-carboxamide (3.0 g, 13.9 mmol, 1 equiv., supplied by Combi-Blocks Inc.), chloroformamidine hydrochloride (3192.9 mg, 27.8 mmol, 2 equiv.), methyl sulfone (13.1 g, 139 mmol, 10 equiv.) in sulfolane (1 mL) in a sealed tube, was heated at 165° C. After 24 h, the mixture was diluted with water and then cooled to rt. The reaction was adjusted to pH 12 using NH₄OH and stirred for 20 minutes. The precipitates were then filtered, rinsed with water, hexanes, and ether, and dried in a vacuum oven at 100° C. overnight to afford 82A that was used without further purification. LCMS (m/z): 242.92 [M+H]⁺; t_{R}=0.55 min. on LC/MS Method A.

Synthesis of 2-amino-7-bromopyrido[3,2-d]pyrimidin-4-yl 4-methylbenzenesulfonate (82B). 2-amino-7-bromopyrido[3,2-d]pyrimidin-4-ol 82A (1000 mg, 4.2 mmol, 1 equiv.) was treated with acetonitrile (40 mL) followed by potassium carbonate (1433.4 mg, 10.37 mmol, 2.5 equiv.) and p-toluenesulfonyl chloride (1186.38 mg, 6.22 mmol, 1.5 equiv.). The reaction mixture was heated to 100° C. and stirred overnight. The mixture was allowed to cool and then diluted with EtOAc, washed with water and saturated NH₄Cl. The organic layer was dried over MgSO₄, filtered, and concentrated under reduced pressure to afford 82B that was used without further purification. LCMS (m/z): 396.98 [M+H]⁺; t_{R}=1.15 min. on LC/MS Method A.

Synthesis of (R)-2-((2-amino-7-bromopyrido[3,2-d]pyrimidin-4-yl)amino)hexan-1-ol (82). 2-Amino-7-bromopyrido[3,2-d]pyrimidin-4-yl 4-methylbenzenesulfonate 82B (50 mg, 0.13 mmol, 1 equiv.) was treated with acetonitrile (5 mL), N,N-diisopropylethylamine (0.07 mL, 0.38 mmol, 3 equiv.) and (R)-(−)-2-amino-1-hexanol (44.48 mg, 0.38 mmol, 3 equiv.). After 16 h, the reaction mixture was concentrated under reduced pressure and subjected to reverse phase HPLC (10% to 70% MeCN in water using a Hydro-RP column) to furnish, after collection of product fractions and removal of volatiles in vacuo, 82 as its TFA salt. LCMS (m/z): 342.1 [M+H]⁺; t_{R}=0.90 min. on LC/MS Method A. ¹H NMR (400 MHz, Methanol-d₄) δ 8.69 (d, J=1.9 Hz, 1H), 8.06 (d, J=1.9 Hz, 1H), 4.52 (dq, J=8.7, 5.5 Hz, 1H), 3.86-3.54 (m, 2H), 1.95-1.63 (m, 2H), 1.57-1.29 (m, 5H), 1.11-0.76 (m, 3H). ¹⁹F NMR (377 MHz, Methanol-d₄) δ −77.42.

### Example 83

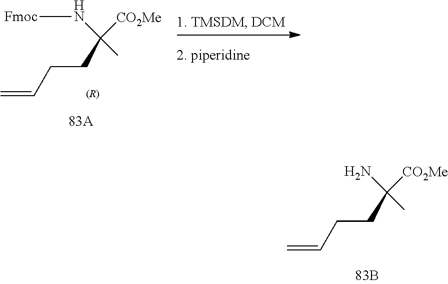

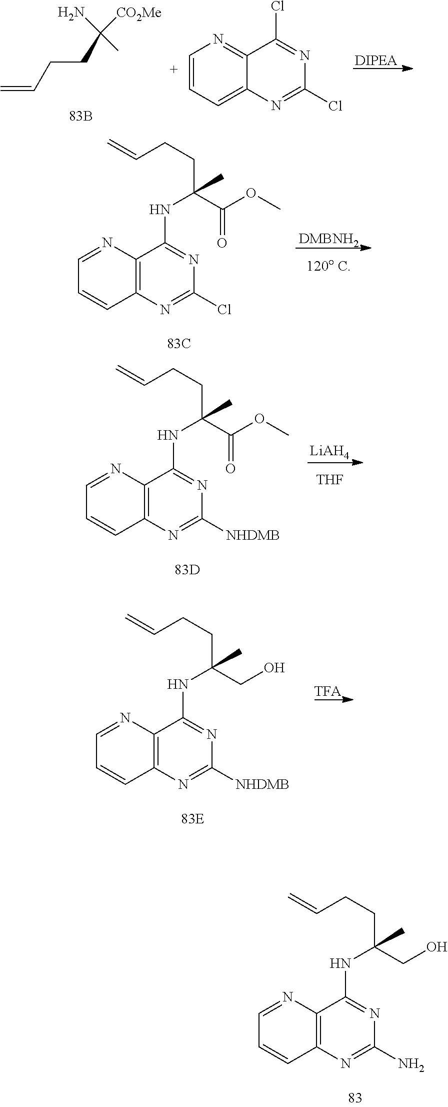

Synthesis of (R)-methyl 2-amino-2-methylhex-5-enoate (83B). (R)-methyl 2-((((9H-fluoren-9-yl)methoxy)carbonyl)amino)-2-methylhex-5-enoate 83A (2 g, 5.5 mmol, 1 equiv., provided by Okeanos Inc.) was treated with DCM (20 mL) and MeOH (4 mL) along with (trimethylsilyl)diazomethane (2.0M solution in hexanes, 4.4 mL, 11.0 mmol, 2.5 equiv.). After 30 minutes, the reaction mixture was concentrated under reduced pressure to provide a residue. The residue was treated with THF (33 mL) followed by piperidine (1.9 mL, 0.02 mol, 3.5 equiv.). The mixture was stirred for 3 days and then concentrated under reduced pressure. The residue was subjected to silica gel chromatography eluting with 0% to 20% MeOH in DCM to provide 83B. LCMS (m/z): 157.91 [M+H]⁺; t_{R}=0.59 min. on LC/MS Method A.

Synthesis of (R)-methyl 2-((2-chloropyrido[3,2-d]pyrimidin-4-yl)amino)-2-methylhex-5-enoate (83C). 2,4-dichloropyrido[3,2-d]pyrimidine (55 mg, 0.28 mmol, 1 equiv.) was treated with dioxane (15 ml) followed by N,N-diisopropylethylamine (0.25 mL, 1.4 mmol, 4 equiv.), and then (R)-methyl 2-amino-2-methylhex-5-enoate 83B (47.6 mg, 0.30 mmol, 1 equiv.). The mixture was stirred at 80° C. overnight to form 83C which was used directly. LCMS (m/z): 321.14 [M+H]⁺; t_{R}=1.21 min. on LC/MS Method A.

Synthesis of (R)-methyl 2-((2-((2,4-dimethoxybenzyl)amino)pyrido[3,2-d]pyrimidin-4-yl)amino)-2-methylhex-5-enoate (83D). (R)-methyl 2-((2-chloropyrido[3,2-d]pyrimidin-4-yl)amino)-2-methylhex-5-enoate 83C solution prepared as described, was treated with 2,4-dimethoxybenzylamine (0.10 mL, 0.69 mmol, 2.5 equiv.). The reaction was heated at 120° C. overnight. The reaction mixture was partitioned between EtOAc (50 mL) and H₂O (50 mL). The organics were separated, dried over MgSO₄, and concentrated in vacuo. The residue was subjected to silica gel chromatography eluting with 0% to 100% EtOAc in hexane to provide 83D. LCMS (m/z): 452.21 [M+H]⁺; t_{R}=1.22 min. on LC/MS Method A.

Synthesis of (R)-2-((2-((2,4-dimethoxybenzyl)amino)pyrido[3,2-d]pyrimidin-4-yl)amino)-2-methylhex-5-en-1-ol (83E). (R)-methyl 2-((2-((2,4-dimethoxybenzyl)amino)pyrido[3,2-d]pyrimidin-4-yl)amino)-2-methylhex-5-enoate 83D (25 mg, 0.06 mmol, 1 equiv.) was treated with THF (20 mL) and 1M lithium aluminum hydride in ether (0.14 mL, 0.14 mmol, 2.5 equiv.). The reaction mixture was stirred for 2 h and then the reaction was quenched with water (100 mL) and extracted with EtOAc (100 mL). The organic layer was separated, dried over MgSO₄, and concentrated in vacuo to provide the 83E that was used without further purification. LCMS (m/z): 424.14 [M+H]⁺; t_{R}=1.12 min. on LC/MS Method A.

Synthesis of (R)-2-((2-aminopyrido[3,2-d]pyrimidin-4-yl)amino)-2-methylhex-5-en-1-ol (83). (R)-2-((2-((2,4-dimethoxybenzyl)amino)pyrido[3,2-d]pyrimidin-4-yl)amino)-2-methylhex-5-en-1-ol 83E (23 mg, 0.05 mmol, 1 equiv.) was treated with DCM (2 mL) and TFA (0.5 mL). After 3 h the reaction mixture was concentrated under reduced pressure and subjected to reverse phase HPLC (10% to 70% MeCN in water with 0.1% TFA using a Hydro-RP column) to furnish, after collection of product fractions and removal of volatiles in vacuo, 83 (10 mg, 65%) as its TFA salt. LCMS (m/z): 274.7 [M+H]⁺; t_{R}=0.73 min. on LC/MS Method A. ¹H NMR (400 MHz, Methanol-d₄) δ 9.01 (d, J=4.5 Hz, 1H), 8.33-8.09 (m, 2H), 6.23 (ddt, J=16.4, 11.0, 5.8 Hz, 1H), 5.42 (d, J=17.1 Hz, 1H), 4.40 (d, J=11.3 Hz, 1H), 4.26-4.03 (m, 2H), 2.57 (ddd, J=29.2, 14.7, 8.4 Hz, 3H), 2.42 (dq, J=10.9, 6.9 Hz, 1H), 1.96 (s, 3H). ¹⁹F NMR (377 MHz, Methanol-d₄) δ −77.19 (d, J=144.5 Hz).

### Example 84

### Synthesis of Intermediate Compound 84E

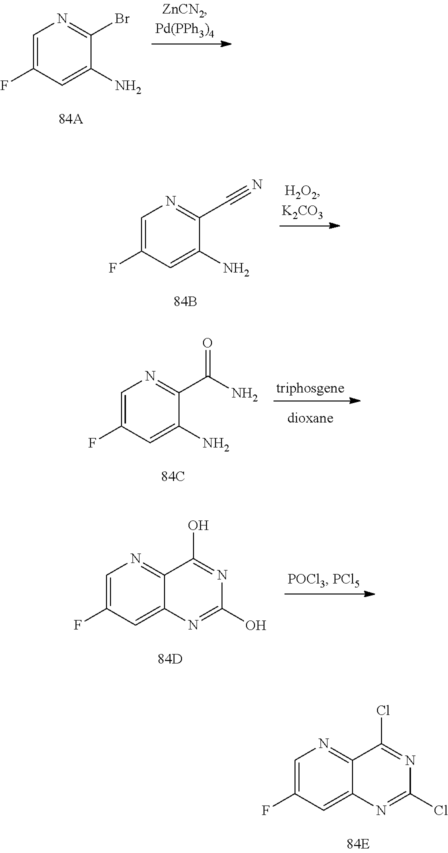 [SMILES: Nc1cc(F)cnc1Br ; N#Cc1ncc(F)cc1N ; NC(=O)c1ncc(F)cc1N ; Oc1nc(O)c2ncc(F)cc2n1 ; Fc1cnc2c(Cl)nc(Cl)nc2c1]

Synthesis of 3-amino-5-fluoropicolinonitrile (84B). 3-amino-2-bromo-5-fluoropyridine 84A (25 g, 131 mmol, Astatech Chemical, Inc) was treated with ZnCN₂ (16.9 g, 1.1 equiv., 144 mmol), Pd(Ph₃)₄ (11.3 g, 0.075 equiv., 9.8 mmol) and DMF (200 mL) and then heated to 115° C. After 6 h, the reaction mixture was allowed to cool and then concentrated under reduced pressure to a solid. The solid was washed with EtOAc (2×100 mL). The organic layers were combined and washed with water (3×100 mL), saturated NH₄Cl solution (100 mL), dried over MgSO₄, filtered and concentrated under reduced pressure to provide 84B that was used without further purification. LCMS (m/z): 138.87 [M+H]⁺; t_{R}=0.59 min. on LC/MS Method A.

Synthesis of 3-amino-5-fluoropicolinamide (84C). Compound 84B (2.6 g, 19.0 mmol, 1 equiv.) was treated with DMSO (10 mL) and cooled to 0° C. before K₂CO₃ (524 mg, 0.2 equiv., 3.8 mmol) was added. H₂O₂ (2.3 mL, 1.2 equiv., 22.8 mmol, 30% water) was then slowly added. The cooling bath was removed and the reaction was stirred for 1 h. The reaction mixture was diluted with water (100 mL) and extracted with EtOAc (3×100). The combined organic layers were washed with water (3×500) and saturated NH₄Cl solution (500 mL), dried over MgSO₄, filtered and concentrated under reduced pressure. The crude material 84C was used without further purification. LCMS (m/z): 155.87 [M+H]⁺; t_{R}=0.62 min. on LC/MS Method A.

The following procedure was adapted from De Jonghe, WO 2006/1359931.

Synthesis of 7-fluoropyrido[3,2-d]pyrimidine-2,4-diol (84D). Carboxamide 84C (1 g, 1 equiv., 6.4 mmol) was treated with triphosgene (1.9 g, 1.0 equiv., 6.4 mmol) and dioxane (20 mL). The reaction mixture was heated to 110° C. for 30 min. The reaction mixture was allowed to cool and concentrated under reduced pressure. The crude solid residue was washed with DCM and diethyl ether and allowed to air dry to provide 84D. LCMS (m/z): 181.95 [M+H]⁺; t_{R}=0.62 min. on LC/MS Method A.

Synthesis of 2,4-dichloro-7-fluoropyrido[3,2-d]pyrimidine (84E). Dione 84D (13.7 g, 75.6 mmol, 1 equiv.) was treated with phosphorus pentachloride (63.0 g, 302.6 mmol, 4 equiv.) and phosphorus (V) oxychloride (141 mL, 20 equiv.) and heated to 110° C. under a under reflux condenser for 8 h. The reaction mixture was concentrated under reduced pressure and azeotroped with toluene. The resultant solid was treated with EtOAc (500 mL) and ice-water (500 mL). The organic layer was separated and washed with saturated NaHCO₃ solution (500 mL), water (500 mL), and saturated NH₄Cl (500 mL). The organic solution was dried over MgSO₄, filtered and concentrated under reduced pressure to furnish the crude product 84E. LCMS (m/z): 213.9 [M+H+2(OMe)-2Cl]⁺; t_{R}=0.82 min. on LC/MS Method A. ¹H NMR (400 MHz, Chloroform-d) δ 9.01 (d, J=2.6 Hz, 1H), 7.94 (dd, J=7.9, 2.7 Hz, 1H). ¹⁹F NMR (377 MHz, Chloroform-d) δ −111.79 (d, J=7.9 Hz).

### Synthesis of Compound 84

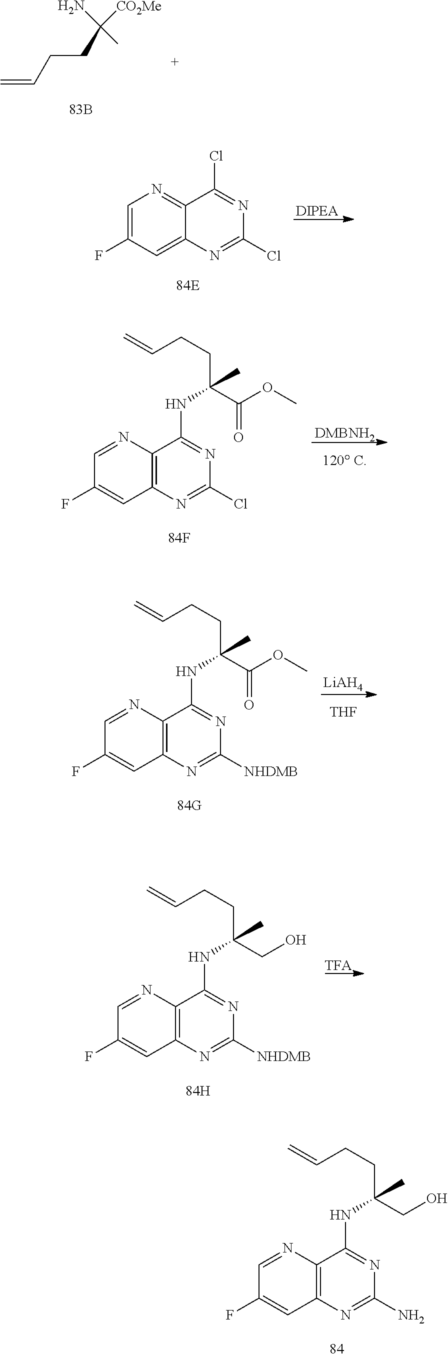

Synthesis of (R)-methyl 2-((2-chloro-7-fluoropyrido[3,2-d]pyrimidin-4-yl)amino)-2-methylhex-5-enoate (84F). 2,4-dichloro-7-fluoropyrido[3,2-d]pyrimidine 84E (75 mg, 0.34 mmol, 1 equiv.) was treated with dioxane (15 ml) followed by N,N-diisopropylethylamine (0.31 mL, 1.7 mmol, 5 equiv.), and then (R)-methyl 2-amino-2-methylhex-5-enoate 83B (59.5 mg, 0.38 mmol, 1 equiv.). The mixture was stirred at 80° C. overnight to form 84F in solution which was then used directly. LCMS (m/z): 339.1 [M+H]⁺; t_{R}=1.23 min. on LC/MS Method A.

Synthesis of (R)-methyl 2-((2-((2,4-dimethoxybenzyl)amino)-7-fluoropyrido[3,2-d]pyrimidin-4-yl)amino)-2-methylhex-5-enoate (84G). (R)-methyl 2-((2-chloro-7-fluoropyrido[3,2-d]pyrimidin-4-yl)amino)-2-methylhex-5-enoate 84F solution prepared as described, was treated with 2,4-dimethoxybenzylamine (0.10 mL, 0.69 mmol, 2.5 equiv.). The reaction was heated at 120° C. overnight. The reaction mixture partitioned between EtOAc (50 mL) and H₂O (50 mL). The organics were separated, dried over MgSO₄, and concentrated in vacuo. The residue was subjected to silica gel chromatography eluting with 0% to 100% EtOAc in hexane to provide 84G. LCMS (m/z): 470.25 [M+H]⁺; t_{R}=1.12 min. on LC/MS Method A.

Synthesis of (R)-2-((2-((2,4-dimethoxybenzyl)amino)-7-fluoropyrido[3,2-d]pyrimidin-4-yl)amino)-2-methylhex-5-en-1-ol (84H). (R)-methyl 2-((2-((2,4-dimethoxybenzyl)amino)-7-fluoropyrido[3,2-d]pyrimidin-4-yl)amino)-2-methylhex-5-enoate 83G (85 mg, 0.18 mmol, 1 equiv.) was treated with THF (5 mL) and 1M lithium aluminum hydride in ether (0.54 mL, 0.54 mmol, 3 equiv.). The reaction mixture was stirred for 2 h and then the reaction was quenched with water (100 mL) and extracted with EtOAc (100 mL). The organic layer was separated, dried over MgSO₄, and concentrated in vacuo to provide 84H that was used without further purification. LCMS (m/z): 442.16 [M+H]⁺; t_{R}=1.07 min. on LC/MS Method A.

Synthesis of (R)-2-((2-amino-7-fluoropyrido[3,2-d]pyrimidin-4-yl)amino)-2-methylhex-5-en-1-ol (84). (R)-2-((2-((2,4-dimethoxybenzyl)amino)-7-fluoropyrido[3,2-d]pyrimidin-4-yl)amino)-2-methylhex-5-en-1-ol 84H (35 mg, 0.08 mmol, 1 equiv.) was treated with DCM (2 mL) and TFA (0.5 mL). After 3 h the reaction mixture was concentrated under reduced pressure and subjected to reverse phase HPLC (10% to 70% MeCN in water with 0.1% TFA using a Hydro-RP column) to furnish, after collection of product fractions and removal of volatiles in vacuo, as its TFA salt. LCMS (m/z): 292.13 [M+H]⁺; t_{R}=0.62 min. on LC/MS Method A. ¹H NMR (400 MHz, Methanol-d₄) δ 8.55 (d, J=2.4 Hz, 1H), 8.25 (s, 1H), 7.63 (dd, J=8.7, 2.5 Hz, 1H), 5.83 (ddt, J=16.6, 10.2, 6.2 Hz, 1H), 5.02 (dq, J=17.1, 1.5 Hz, 1H), 4.92 (ddt, J=10.2, 2.1, 1.1 Hz, 8H), 4.08-3.88 (m, 1H), 3.69 (d, J=11.3 Hz, 1H), 2.34-1.90 (m, 4H), 1.56 (s, 3H). ¹⁹F NMR (377 MHz, Methanol-d₄) δ −77.54, −118.17 (dd, J=8.8, 4.3 Hz).

### Example 85

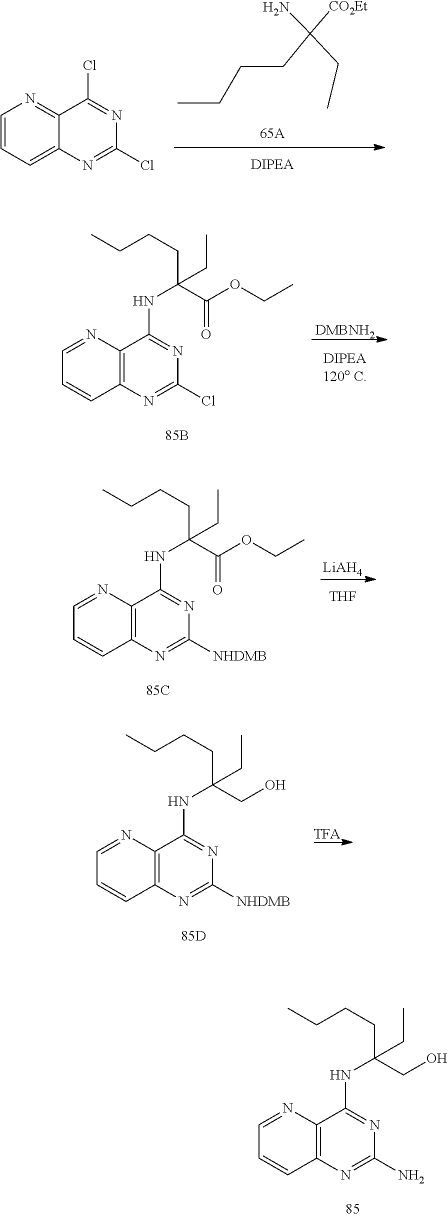

Synthesis of ethyl 2-((2-chloropyrido[3,2-d]pyrimidin-4-yl)amino)-2-ethylhexanoate (85B). 2,4-dichloropyrido[3,2-d]pyrimidine (1068 mg, 5.34 mmol, 1 equiv.) was treated with dioxane (10 ml) followed by N,N-diisopropylethylamine (5.7 mL, 32.0 mmol, 6 equiv.), and then 2-amino-2-ethyl-hexanoic acid ethyl ester 85A (1000 mg, 5.34 mmol, 1 equiv., supplied by J&W Pharmlab, LLC). The mixture was stirred at 80° C. overnight. The reaction mixture partitioned between EtOAc (50 mL) and H₂O (50 mL). The organics were separated, dried over MgSO₄, and concentrated in vacuo to afford 85B that was then used directly. LCMS (m/z): 351.23 [M+H]⁺; t_{R}=1.43 min. on LC/MS Method A.

Synthesis of ethyl 2-((2-((2,4-diethylbenzyl)amino)pyrido[3,2-d]pyrimidin-4-yl)amino)-2-ethylhexanoate (85C). Ethyl 2-((2-chloropyrido[3,2-d]pyrimidin-4-yl)amino)-2-ethylhexanoate 85B prepared as described, was treated with dioxane (10 mL), N,N-diisopropylethylamine (1.7 mL, 9.5 mmol, 3 equiv.), and 2,4-dimethoxybenzylamine (0.94 mL, 6.3 mmol, 2 equiv.). The reaction was heated at 120° C. overnight. The reaction mixture partitioned between EtOAc (50 mL) and H₂O (50 mL). The organics were separated, dried over MgSO₄, and concentrated in vacuo. The residue was subjected to silica gel chromatography eluting with 0% to 100% EtOAc in hexane to provide 85C. LCMS (m/z): 482.27 [M+H]⁺; t_{R}=1.02 min. on LC/MS Method A.

Synthesis of 2-((2-((2,4-diethylbenzyl)amino)pyrido[3,2-d]pyrimidin-4-yl)amino)-2-ethylhexan-1-ol (85D). Ethyl 2-((2-((2,4-diethylbenzyl)amino)pyrido[3,2-d]pyrimidin-4-yl)amino)-2-ethylhexanoate 85C (111 mg, 0.23 mmol, 1 equiv.) was treated with THF (10 mL) and 1M lithium aluminum hydride in ether (0.92 mL, 0.92 mmol, 4 equiv.). The reaction mixture was stirred for 2 h and then the reaction was quenched with water (100 mL) and extracted with EtOAc (100 mL). The organic layer was separated, dried over MgSO₄, and concentrated in vacuo. The residue was subjected to silica gel chromatography eluting with 0% to 100% EtOAc in hexane to provide 85D. LCMS (m/z): 440.24 [M+H]⁺; t_{R}=0.94 min. on LC/MS Method A.

Synthesis of 2-((2-aminopyrido[3,2-d]pyrimidin-4-yl)amino)-2-ethylhexan-1-ol (85). 2-((2-((2,4-Diethylbenzyl)amino)pyrido[3,2-d]pyrimidin-4-yl)amino)-2-ethylhexan-1-ol 85D (16 mg, 0.04 mmol, 1 equiv.) was treated with DCM (2 mL) and TFA (0.5 mL). After 6 h the reaction mixture was concentrated under reduced pressure and subjected to reverse phase HPLC (10% to 70% MeCN in water with 0.1% TFA using a Hydro-RP column) to furnish, after collection of product fractions and removal of volatiles in vacuo, 85 as its TFA salt. LCMS (m/z): 290.15 [M+H]⁺; t_{R}=0.73 min. on LC/MS Method A. ¹H NMR (400 MHz, Methanol-d₄) δ 8.62 (dd, J=4.4, 1.4 Hz, 1H), 7.93-7.61 (m, 2H), 3.98 (s, 3H), 3.91 (s, 2H), 2.10-1.82 (m, 4H), 1.46-1.20 (m, 4H), 1.10-0.71 (m, 5H). ¹⁹F NMR (377 MHz, Methanol-d₄) δ −77.69 (d, J=231.2 Hz).

### Example 86

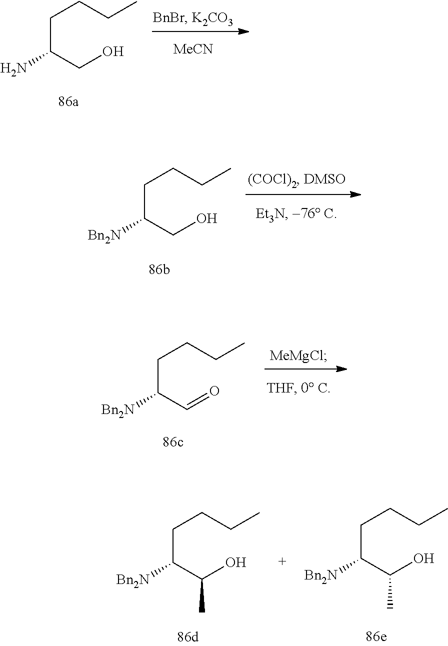

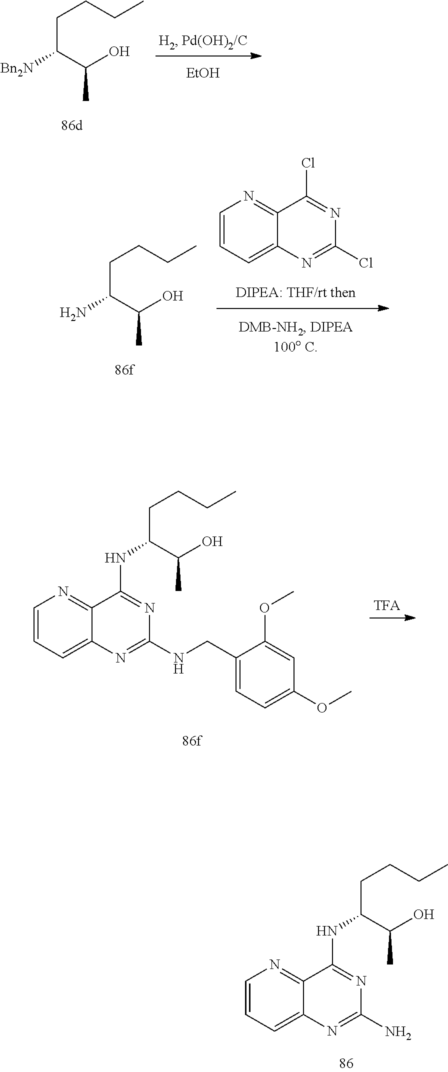

Synthesis of (R)-2-(Dibenzylamino)hexan-1-ol (86b). (R)-norleucinol (86a, 2046.4 mg, 17.46 mmol) was treated with acetonitrile (40 mL) and K₂CO₃ (4842.4 mg, 35.04 mmol) followed by benzyl bromide (6.222 mL, 52.39 mmol) at 0° C. The resulting mixture was stirred at rt. After 18 h, the precipitate was filtered and the solids were washed with EtOAc (30 mL). Filtrates were concentrated under reduced pressure and the resultant residue was subjected to silica gel chromatography eluting with 0-70% EtOAc in hexanes to provide 86b LCMS-ESI⁺ (m/z): [M+H]⁺ calculated for C₂₀H₂₈NO: 298.22; found: 298.16; t_{R}=0.82 min on LC/MS Method A.

Synthesis of (R)-2-(dibenzylamino)hexanal (86c). Oxalyl chloride (0.18 mL, 2.10 mmol) in DCM (3 mL) was cooled in an acetone-dry ice bath and then treated with DMSO (0.3 mL, 4.22 mmol) in DCM (1 mL) dropwise over 2 minutes. After 10 min, a solution of compound 86b (503.5 mg, 1.69 mmol) in DCM (2 mL) was added and resulting mixture was allowed to stir for 30 min. before addition of triethylamine (1.2 mL, 8.61 mmol). After 1 h at −70˜−55° C., the reaction mixture was allowed to warm to rt, diluted with EtOAc (30 mL), and washed with water (30 mL×2). The aqueous fractions were extracted with EtOAc (×1), and the combined organic fractions were then dried (MgSO₄), concentrated under reduced pressure, and the residue vacuum dried to obtain compound 86c, which was used without further purification. LCMS-ESI⁺ (m/z): [M+H]⁺ calculated for C₂₀H₂₆NO: 296.20; found: 296.16; t_{R}=1.12 min on LC/MS Method A.

Synthesis of (2S,3R)-3-(Dibenzylamino)heptan-2-ol (86d) and (2R,3R)-3-(Dibenzylamino)heptan-2-ol (86e). Compound 86c (134.87 mg, 0.457 mmol) in diethyl ether (4 mL) was stirred at −15° C. and a 1.6 M solution of methyl lithium in diethyl ether (4.2 mL, 6.72 mmol) was added. After 0.5 h, the reaction mixture was quenched with saturated aqueous ammonium chloride (10 mL) and water (10 mL), and the product was extracted with EtOAc (20 mL×2). The organic extracts were washed with water (20 mL×1), combined, dried (MgSO₄), and then concentrated under reduced pressure. The crude residue was subjected to silica gel chromatography eluting with 5-30% EtOAc in hexanes to obtain 86d (first eluting compound) and compound 86e second eluting compound.

(2S,3R)-3-(Dibenzylamino)heptan-2-ol (86d): ¹H NMR (400 MHz, Chloroform-d) δ 7.37-7.17 (m, 10H), 4.33 (s, 1H), 3.86 (d, J=13.3 Hz, 1.9H), 3.73 (d, J=13.7 Hz, 0.1H), 3.67-3.55 (m, 1H), 3.45 (d, J=13.3 Hz, 2H), 2.64 (d, J=5.8 Hz, 0.05H), 2.33 (dt, J=9.3, 5.5 Hz, 0.95H), 1.72 (ddd, J=14.8, 12.0, 6.5 Hz, 1H), 1.50-1.20 (m, 6H), 1.18 (d, J=6.7 Hz, 0.15H), 1.09 (d, J=6.0 Hz, 2.85H), 0.96 (t, J=7.1 Hz, 3H). LCMS-ESI+(m/z): [M+H]⁺ calculated for C₂₁H₃₀NO: 312.23; found: 312.16; t_{R}=0.98 min on LC/MS Method A.

(2R,3R)-3-(Dibenzylamino)heptan-2-ol (86e): ¹H NMR (400 MHz, Chloroform-d) δ 7.44-7.13 (m, 10H), 3.88 (dt, J=8.6, 5.8 Hz, 1H), 3.73 (d, J=13.6 Hz, 2H), 3.63 (d, J=13.6 Hz, 2H), 2.65 (td, J=6.5, 4.3 Hz, 1H), 2.31 (s, 1H), 1.73 (td, J=11.0, 9.8, 5.8 Hz, 1H), 1.50-1.22 (m, 6H), 1.18 (d, J=6.6 Hz, 3H), 0.92 (t, J=7.0 Hz, 3H). LCMS-ESI⁺ (m/z): [M+H]⁺ calculated for C₂₁H₃₀NO: 312.23; found: 312.16; t_{R}=0.93 min on LC/MS Method A.

Synthesis of (2S,3R)-3-aminoheptan-2-ol (86f). Diastereomer 86d (108.9 mg, 0.349 mmol) and 20% palladium hydroxide on carbon (25.3 mg) in EtOH (4 mL) was stirred under H₂ atmosphere for 16 h. The resulting mixture was filtered and the filtrate was concentrated under reduced pressure to provide compound 86f contaminated with some EtOH, which was used without further purification. ¹H NMR (400 MHz, Methanol-d₄) δ 3.51 (p, J=6.3 Hz, 1H), 2.49 (ddd, J=8.2, 6.0, 4.0 Hz, 1H), 1.57-1.20 (m, 6H), 1.15 (d, J=6.4 Hz, 3H), 0.97-0.87 (m, 3H).

Synthesis of (2S,3R)-3-((2-((2,4-dimethoxybenzyl)amino)pyrido[3,2-d]pyrimidin-4-yl)amino)heptan-2-ol (86g). Compound 86f prepared as described and 2,4-dichloropyrido[3,2-d]pyrimidine (73.2 mg, 0.350 mmol, Astatech, Inc.) in THF (3 mL) were treated with N,N-diisopropylethylamine (0.19 mL, 1.091 mmol) and the resulting mixture stirred for 1.5 h. Additional THF (3 mL), N,N-diisopropylethylamine (0.19 mL, 1.091 mmol), and 2,4-dimethoxybenzylamine (0.27 mL, 1.797 mmol) were added. The reaction mixture was stirred at 100° C. for 15.5 h and then cooled to rt. The reaction mixture was diluted with DCM (30 mL), washed with water (30 mL×2). The aqueous fractions were then extracted with DCM (20 mL×1), and the combined organic fractions, dried (MgSO₄), and concentrated in vacuo. The residue was subjected to silica gel chromatography eluting with 0-20% methanol in DCM to provide crude 86g. The crude 86g was further subjected to preparative HPLC (Gemini 10u C18110A, AXIA; 10% aq. acetonitrile-80% aq. acetonitrile with 0.1% TFA, over 20 min. gradient). The collected fractions were neutralized with NaHCO₃ before concentration. The residue was dissolved in EtOAc, washed with water, dried (MgSO₄), and concentrated under reduced pressure to provide compound 86g. LCMS-ESI⁺ (m/z): [M+H]⁺ calculated for C₂₃H₃₂N₅O₃: 426.25; found: 426.14; t_{R}=1.23 min on LC/MS Method A.

Synthesis of (2S,3R)-3-(2-aminopyrido[3,2-d]pyrimidin-4-yl)amino)heptan-2-ol (86). Compound 86g (76.0 mg, 0.179 mmol) was dissolved in TFA (2 mL) and stirred at rt for 1 h. The reaction mixture was concentrated and co-evaporated with methanol (10 mL×1). The resulting residue was dissolved in methanol (2 mL) and concentrated ammonium hydroxide (0.2 mL) was added to the solution. After 10 min. at rt, the mixture was concentrated to dryness, and the residue was dissolved in methanol (3 mL) and water (3 mL). The insoluble material was removed by filtration, and the filtrate was subjected to preparative HPLC (Gemini 10u C18110A, AXIA; 10% aq. acetonitrile-70% aq. acetonitrile with 0.1% TFA, over 20 min. gradient) to provide, after collection of product fractions and removal of volatiles in vacuo, compound 86 as its TFA salt. ¹H NMR (400 MHz, Methanol-d₄) δ 8.64 (dd, J=4.4, 1.4 Hz, 1H), 7.84 (dd, J=8.5, 1.5 Hz, 1H), 7.77 (dd, J=8.5, 4.4 Hz, 1H), 4.37 (td, J=7.2, 3.4 Hz, 1H), 3.99 (qd, J=6.4, 3.4 Hz, 1H), 1.76 (q, J=7.4 Hz, 2H), 1.48-1.26 (m, 4H), 1.18 (d, J=6.4 Hz, 3H), 0.97-0.82 (m, 3H). LCMS-ESI⁺ (m/z): [M+H]⁺ calculated for C₁₄H₂₂N₅O: 276.18; found: 276.15; t_{R}=0.67 min on LC/MS Method A.

### Example 87

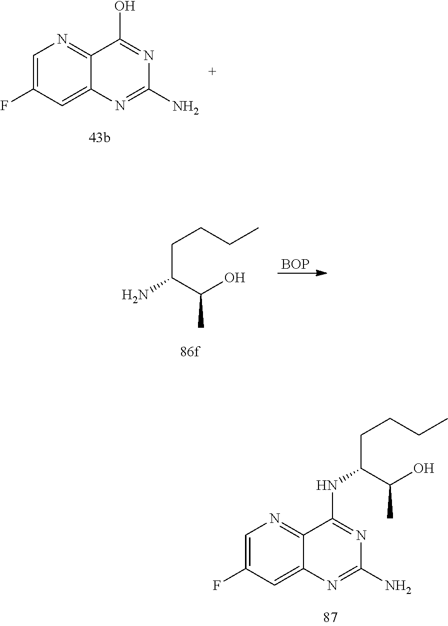

Synthesis of (2S,3R)-3-((2-amino-7-fluoropyrido[3,2-d]pyrimidin-4-yl)amino)heptan-2-ol (87). A solution of 2-amino-7-fluoropyrido[3,2-d]pyrimidin-4-ol (43B, 20.0 mg, 0.068 mmol), compound 86f (27.2 mg, 0.207 mmol), and (benzotriazol-1-yloxy)tris(dimethylamino)phosphonium hexafluorophosphate (BOP, 58.9 mg, 0.133 mmol) in DMF (3 mL) was stirred at rt and 1,8-diazabicyclo[5.4.0]undec-7-ene (DBU, 0.05 mL, 0.333 mmol) was added. After 24 h stirring at rt, the reaction mixture was diluted with water (2 mL) and 1 N HCl (1 mL), and the resulting solution filtered. The filtrate was subjected to preparative HPLC (Gemini 10u C18110A, AXIA; 10% aq. acetonitrile-70% aq. acetonitrile with 0.1% TFA, over 20 min. gradient). The concentrated fractions containing product were concentrated, co-evaporated with methanol (10 mL×3), and then dried in vacuo to obtain compound 87 as its TFA salt. ¹H NMR (400 MHz, Methanol-d₄) δ 8.56 (d, J=2.4 Hz, 1H), 7.64 (dd, J=8.8, 2.4 Hz, 1H), 4.36 (td, J=7.2, 3.6 Hz, 1H), 4.03-3.91 (m, 1H), 1.82-1.69 (m, 2H), 1.37 (tddd, J=12.8, 10.3, 7.7, 5.0 Hz, 4H), 1.18 (d, J=6.4 Hz, 3H), 0.94-0.85 (m, 3H). ¹⁹F NMR (376 MHz, Methanol-d₄) δ −77.82, −117.98 (d, J=8.8 Hz). LCMS-ESI⁺ (m/z): [M+H]⁺ calculated for C₁₄H₂₁FN₅O: 294.17; found: 294.13; t_{R}=0.71 min on LC/MS Method A.

### Example 88

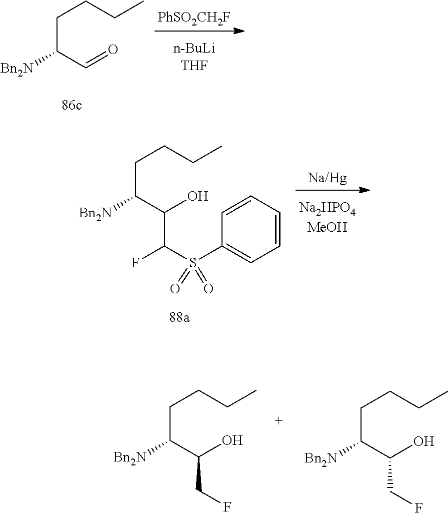

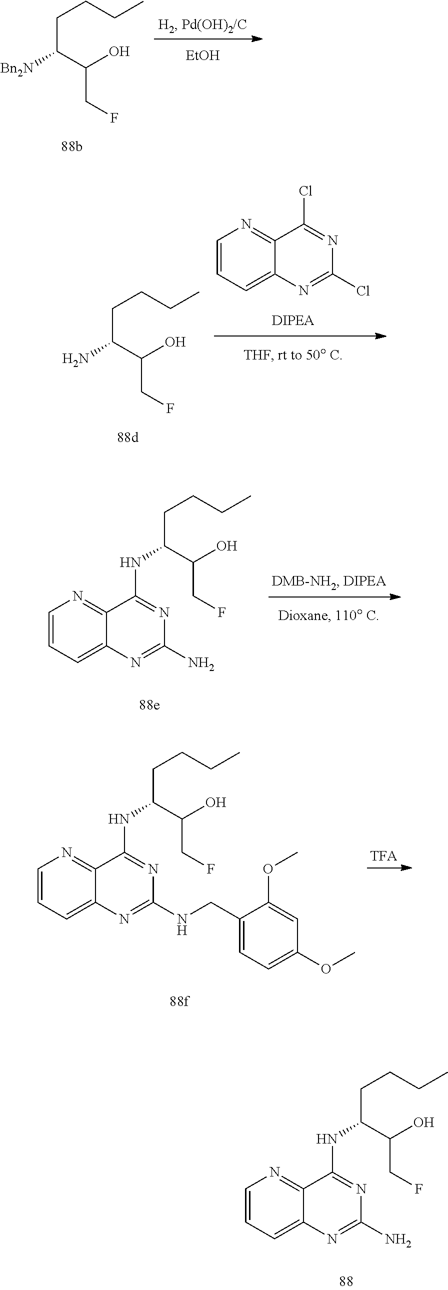

Synthesis of (3R)-3-(dibenzylamino)-1-fluoro-1-(phenylsulfonyl)heptan-2-ol (88a). A solution of fluoromethyl phenyl sulphone (935.6 mg, 5.371 mmol) in THF (3 mL) was stirred in an acetone-dry ice bath and 2.5 M n-butyllithium in hexane (2.15 mL) was added. After 30 min, the crude compound 86c (393.9 mg, 1.333 mmol) in THF (2 mL) was added and the resulting solution stirred with cooling by an acetone-dry ice bath. After 30 minutes, the reaction mixture was quenched with saturated NH₄Cl (15 mL), diluted with EtOAc (30 mL), and warmed up to rt before the two fractions were separated. The aqueous fraction was extracted with EtOAc (20 mL×1), and the organic fractions were then washed with water (30 mL×1), before being combined, dried (MgSO₄), and concentrated under reduced pressure. The residue was subjected to silica gel chromatography eluting with 0-40% EtOAc in hexanes to provide compound 88a, as a mixture of 4 diastereomers. LCMS-ESI⁺ (m/z): [M+H]⁺ calculated for C₂₇H₃₃FNO₃S: 470.22; found: 470.24; t_{R}=1.40-1.45 min.

Synthesis of (2R,3R)-3-(dibenzylamino)-1-fluoroheptan-2-ol and (2S,3R)-3-(dibenzylamino)-1-fluoroheptan-2-ol (88b and 88c). A suspension of compound 88a (635.4 mg, 1.333 mmol) and Na₂HPO₄ (1325.9 mg, 9.340 mmol) in methanol (10 mL) was stirred in −30˜−40° C. bath as sodium-mercury amalgam (1853.9 mmol, 8.060 mmol) was added. The reaction mixture was slowly warmed to ˜5° C. over 2 h and then stirred 1 h at ˜5° C. The mixture was then filtered through a Celite pad and the filtrate was concentrated in vacuo. The residue was dissolved in EtOAc and water (20 mL each), and the two fractions separated. The aqueous fraction was extracted with EtOAc (20 mL×1). The organic fractions were washed with water (30 mL×1), then combined, dried (MgSO4), and concentrated under reduced pressure. The residue was subjected to repeated silica gel chromatography eluting with 5-20% EtOAc in hexanes to provide compound 88b, as the first eluting fraction, and compound 88c as the second eluting fraction.

Compound 88b: ¹H NMR (400 MHz, Chloroform-d) δ 7.63-6.91 (m, 10H), 4.53-4.27 (m, 2H), 4.16 (s, 1H), 3.90 (d, J=13.2 Hz, 2H), 3.66 (dt, J=22.5, 5.7 Hz, 1H), 3.49 (d, J=13.3 Hz, 2H), 2.69 (dt, J=9.2, 5.3 Hz, 1H), 1.90-1.70 (m, 1H), 1.39 (tdd, J=12.6, 8.2, 5.5 Hz, 5H), 0.97 (t, J=7.0 Hz, 3H). ¹⁹F NMR (376 MHz, Chloroform-d) δ −230.59 (td, J=47.8, 23.5 Hz). LCMS-ESI⁺ (m/z): [M+H]⁺ calculated for C₂₁H₂₉FNO: 330.22; found: 330.17; t_{R}=0.96 min on LC/MS Method A.

Compound 88c: ¹H NMR (400 MHz, Chloroform-d) δ 7.54-6.94 (m, 10H), 4.54 (ddd, J=47.2, 9.4, 3.4 Hz, 1H), 4.25 (ddd, J=48.2, 9.4, 7.3 Hz, 1H), 4.01 (d, J=18.6 Hz, 1H), 3.66 (d, J=2.5 Hz, 4H), 2.68 (q, J=6.1 Hz, 1H), 2.35 (s, 1H), 1.88-1.70 (m, 1H), 1.53-1.21 (m, 5H), 1.00-0.80 (m, 3H). ¹⁹F NMR (376 MHz, Chloroform-d) δ −228.21 (td, J=47.7, 18.4 Hz). LCMS-ESI⁺ (m/z): [M+H]⁺ calculated for C₂₁H₂₉FNO: 330.22; found: 330.13; t_{R}=1.07 min on LC/MS Method A.

Synthesis of (3R)-3-amino-1-fluoroheptan-2-ol (88d). A mixture of compound 88b (38.25 mg, 0.116 mmol) and 20% palladium hydroxide on carbon (15.61 mg) in EtOH (2 mL) was stirred under H₂ atmosphere. After 20.5 h, the reaction mixture was filtered and the solids washed with EtOH (10 mL). After the filtrate and washing was concentrated, the residue was co-evaporated with toluene (5 mL×2) to obtain compound 88d. LCMS-ESI⁺ (m/z): [M+H]⁺ calculated for C₇H₁₇FNO: 150.13; found: 149.97; t_{R}=0.40 min on LC/MS Method A.

Synthesis of (3R)-3-((2-chloropyrido[3,2-d]pyrimidin-4-yl)amino)-1-fluoroheptan-2-ol (88e). To a solution of compound 88d (14.9 mg, 0.100 mmol) and 2,4-dichloropyrido[3,2-d]pyrimidine (11.6 mg, 0.158 mmol) in THF (2 mL) was added N,N-diisopropylethylamine (0.1 mL, 0.574 mmol). The mixture was stirred at rt for 1.5 h and at 50° C. for 30 min. The reaction mixture was then concentrated in vacuo, and the residue subjected to silica gel chromatography eluting with 20-70% EtOAc in hexanes to obtain compound 88e. LCMS-ESI⁺ (m/z): [M+H]⁺ calculated for C₁₄H₁₉ClFN₄O: 313.12; found: 313.14; t_{R}=1.06 min on LC/MS Method A.

Synthesis of (3R)-3-((2-((2,4-dimethoxybenzyl)amino)pyrido[3,2-d]pyrimidin-4-yl)amino)-1-fluoroheptan-2-ol (88f). To solution of compound 88e (22.0 mg, 0.070 mmol) in dioxane (2 mL), N,N-diisopropylethylamine (0.06 mL, 0.344 mmol), and 2,4-dimethoxybenzylamine (0.04 mL, 0.266 mmol) were added. The resulting solution was refluxed at 110° C. for 19 h. After the reaction mixture was concentrated, the residue was subjected to silica gel chromatography eluting with hexanes-EtOAc to provide crude product 88f. The crude product was then subjected to preparative HPLC (Gemini 10u C18110A, AXIA; 10% aq. acetonitrile-80% aq. acetonitrile with 0.1% TFA, over 20 min. gradient). The combined product fractions were neutralized by the addition of saturated aqueous NaHCO₃ (1 mL), concentrated to remove acetonitrile, and then extracted with EtOAc (20 mL×2). The organic extracts were washed with water (×1), combined, dried (MgSO₄), and concentrated under reduced pressure to obtain compound 88f LCMS-ESI⁺ (m/z): [M+H-C₂H₄]⁺ calculated for C₂₃H₃₁FN₅O₃: 444.24; found: 444.18; t_{R}=0.95 min on LC/MS Method A.

Synthesis of (3R)-3-((2-aminopyrido[3,2-d]pyrimidin-4-yl)amino)-1-fluoroheptan-2-ol (88). Compound 88f (8.7 mg, 30.44 umol) was dissolved in TFA (1 mL) and stirred at rt for 1 h. The reaction mixture was concentrated in vacuo and co-evaporated with methanol (10 mL). The residue was dissolved in methanol (1 mL) and concentrated ammonium hydroxide (0.1 ML) was added. The resulting mixture was stirred at rt for 10 min, concentrated under reduced pressure. The residue was triturated in 1 N HCl (0.5 mL) and methanol (2 mL), filtered, and diluted with water (3 mL) before subjecting to preparative HPLC (Gemini 10u C18110A, AXIA; 10% aq. acetonitrile-70% aq. acetonitrile with 0.1% TFA, over 20 min. gradient). The product fractions were combined, concentrated in vacuo, co-evaporated with methanol (10 mL×3) and dried in vacuo to obtain compound 88 as its TFA salt. ¹H NMR (400 MHz, Methanol-d₄) δ 8.64 (dd, J=4.4, 1.4 Hz, 1H), 7.84 (dd, J=8.5, 1.4 Hz, 1H), 7.77 (dd, J=8.5, 4.4 Hz, 1H), 4.59 (ddd, J=8.0, 6.5, 3.0 Hz, 1H), 4.51-4.38 (m, 1H), 4.38-4.26 (m, 1H), 4.04 (dddd, J=16.2, 6.1, 4.9, 3.1 Hz, 1H), 1.89-1.73 (m, 2H), 1.39 (dtd, J=10.4, 6.9, 6.3, 3.4 Hz, 4H), 0.96-0.84 (m, 3H). ¹⁹F NMR (376 MHz, Methanol-d₄) δ −77.56, −231.26 (td, J=47.3, 16.2 Hz). LCMS-ESI⁺ (m/z): [M+H]⁺ calculated for C₁₄H₂₁FN₅O: 294.17; found: 294.15; t_{R}=0.69 min on LC/MS Method A.

### Example 89

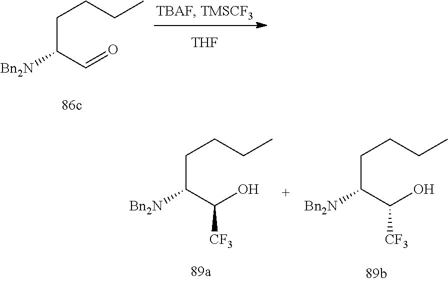

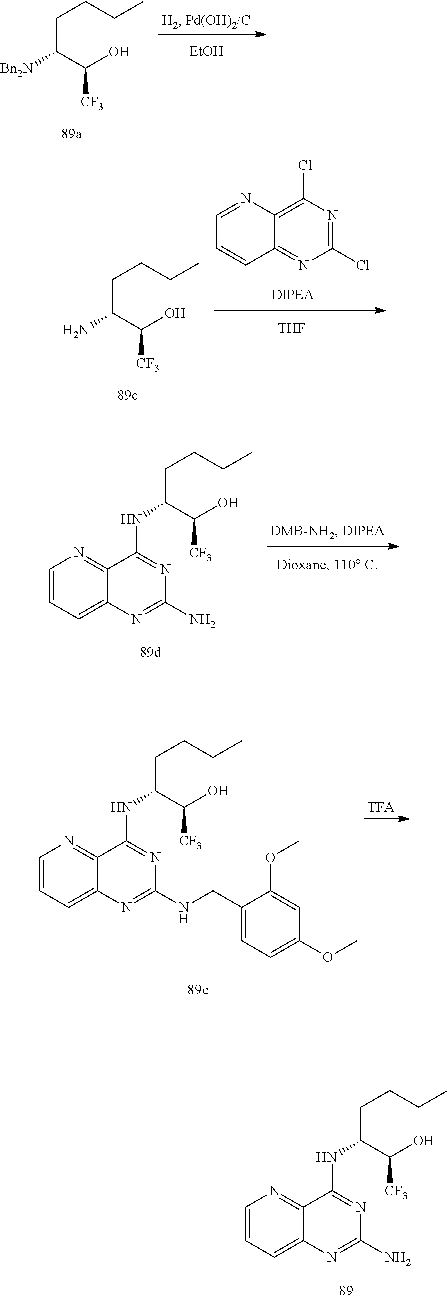

Synthesis of (2S,3R)-3-(dibenzylamino)-1,1,1-trifluoroheptan-2-ol (89a) and (2R,3R)-3-(dibenzylamino)-1,1,1-trifluoroheptan-2-ol (89b). A solution of compound 86c (492.7 mg, 1.668 mmol) and tetrabutylammonium fluoride (TBAF, 21.8 mg, 0.083 mmol) in THF (4 mL) was stirred at 0° C. and trimethyl(trifluoromethyl)silane (0.76 mL, 5.17 mmol) was added. After the resulting mixture was stirred at 0° C. for 30 min, additional TBAF (87.2 mg, 0.334 mmol) was added and the reaction mixture was stirred for 1h at rt. The reaction mixture was quenched with saturated aqueous NH₄Cl (10 mL). The resulting solution was diluted with EtOAc (20 mL) and two layers were separated. The aqueous fraction was extracted with EtOAc (20 mL×3) and the organic fractions were washed with brine (20 mL×1), combined, dried (MgSO₄), and concentrated in vacuo. The residue was then subjected to silica gel chromatography eluting with 0-20% EtOAc in hexanes to obtain compound 89a, as the first eluting product and compound 89b as the second eluting product.

Compound 89a: ¹H NMR (400 MHz, Chloroform-d) δ 7.36-7.26 (m, 10H), 5.30 (s, 1H), 3.90 (d, J=13.1 Hz, 2H), 3.74-3.64 (m, 1H), 3.60 (d, J=13.1 Hz, 2H), 2.97 (d, J=9.3 Hz, 1H), 1.94-1.80 (m, 1H), 1.60-1.44 (m, 3H), 1.38 (h, J=7.4 Hz, 2H), 0.98 (t, J=7.2 Hz, 3H). ¹⁹F NMR (376 MHz, Chloroform-d) δ −76.57 (d, J=6.3 Hz). LCMS-ESI⁺ (m/z): [M+H]⁺ calculated for C₂₁H₂₇F₃NO: 366.20; found: 366.15; TR=1.46 min.

Compound 89b: ¹H NMR (400 MHz, Chloroform-d) δ 7.32 (d, J=4.8 Hz, 10H), 4.22 (s, 1H), 3.82 (d, J=13.6 Hz, 2H), 3.50 (d, J=13.6 Hz, 2H), 3.00 (d, J=9.4 Hz, 1H), 2.66 (s, 1H), 1.79 (q, J=9.1 Hz, 1H), 1.49 (s, 2H), 1.35-1.11 (m, 4H), 0.87 (t, J=7.2 Hz, 3H). ¹⁹F NMR (376 MHz, Chloroform-d) δ −76.53 (d, J=8.3 Hz). LCMS-ESI⁺ (m/z): [M+H]⁺ calculated for C₂₁H₂₇F₃NO: 366.20; found: 366.15; t_{R}=1.49 min on LC/MS Method A.

Synthesis of (2R,3R)-3-amino-1,1,1-trifluoroheptan-2-ol (89c). To a stirred solution of compound 89a (121.35 mg, 0.332 mmol) in EtOH (4 mL) was added 20% palladium hydroxide on carbon (52 mg, 0.074 mmol). The resulting mixture was stirred under H₂ atmosphere for 20 h. The reaction mixture was then filtered and washed with ethanol (10 mL). The filtrate was then concentrated in vacuo to obtain compound 89c. LCMS-ESI⁺ (m/z): [M+H]⁺ calculated for C₇H₁₅F₃NO: 186.11; found: 185.96; t_{R}=0.55 min on LC/MS Method A.

Synthesis of (2R,3R)-3-((2-chloropyrido[3,2-d]pyrimidin-4-yl)amino)-1,1,1-trifluoroheptan-2-ol (89d). To a solution of compound 89c (53.4 mg, 0.288 mmol) and 2,4-dichloropyrido[3,2-d]pyrimidine (57.68 mg, 0.288 mmol) in THF (3 mL) was added N,N-diisopropylethylamine (0.151 mL, 0.865 mmol) and the mixture heated to 80° C. After 2 h, the reaction mixture was allowed to cool to rt and then concentrated in vacuo and the residue subjected to silica gel chromatography eluting with 0-100% EtOAc in hexanes to afford compound 89d.

Synthesis of (2R,3R)-3-((2-((2,4-dimethoxybenzyl)amino)pyrido[3,2-d]pyrimidin-4-yl)amino)-1,1,1-trifluoroheptan-2-ol (89e). To a solution of compound 89d (106.7 mg, 0.346 mmol) in dioxane (3 mL) was added N,N-diisopropylethylamine (0.160 mL, 0.918 mmol) and 2,4-dimethoxybenzylamine (0.230 mL, 1.530 mmol). The resulting solution was refluxed at 110° C. and stirred for 20 h. The reaction mixture was then cooled to rt and diluted with EtOAc (20 mL), washed with water (20 mL×3) and brine (20 mL×1), dried (MgSO₄), filtered and then concentrated in vacuo. The residue was subjected to silica gel chromatography eluting with 0-100% EtOAc in hexanes to afford compound 89e. LCMS-ESI⁺ (m/z): [M+H-C₂H₄]⁺ calculated for C₂₃H₂₉F₃N₅O₃: 480.22; found: 480.17; t_{R}=1.03 min on LC/MS Method A.

Synthesis of (2R,3R)-3-((2-aminopyrido[3,2-d]pyrimidin-4-yl)amino)-1,1,1-trifluoroheptan-2-ol (89). Compound 89e (12 mg, 25.0 umol) was dissolved in TFA (1 mL) and stirred at rt for 1 h. The reaction mixture was concentrated in vacuo and co-evaporated with methanol (10 mL). The resulting residue was dissolved in aqueous methanol (1 mL), filtered through a Celite-membrane filter to remove insoluble material, and the filtrate subjected to preparative HPLC (Gemini 10u C18110A, AXIA; 10% aq. acetonitrile-70% aq. acetonitrile with 0.1% TFA, over 20 min. gradient). The collected product fractions were concentrated in vacuo, and the residue was co-evaporated with methanol (10 mL×3), and dried in vacuum overnight to obtain compound 89 as its TFA salt. ¹H NMR (400 MHz, Methanol-d₄) δ 8.65 (dd, J=4.4, 1.4 Hz, 1H), 7.85 (dd, J=8.5, 1.4 Hz, 1H), 7.79 (dd, J=8.5, 4.4 Hz, 1H), 4.82 (ddd, J=8.3, 6.5, 2.1 Hz, 1H), 4.22 (qd, J=7.3, 1.9 Hz, 1H), 1.92-1.74 (m, 2H), 1.50-1.31 (m, 4H), 0.96-0.87 (m, 3H). ¹⁹F NMR (376 MHz, Methanol-d₄) δ −77.56, −79.32 (d, J=7.3 Hz). LCMS-ESI⁺ (m/z): [M+H]⁺ calculated for C₁₄H₁₉F₃N₅O: 330.15; found: 330.15; t_{R}=0.77 min on LC/MS Method A.

### Example 90

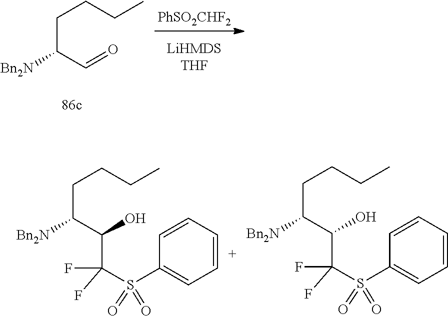

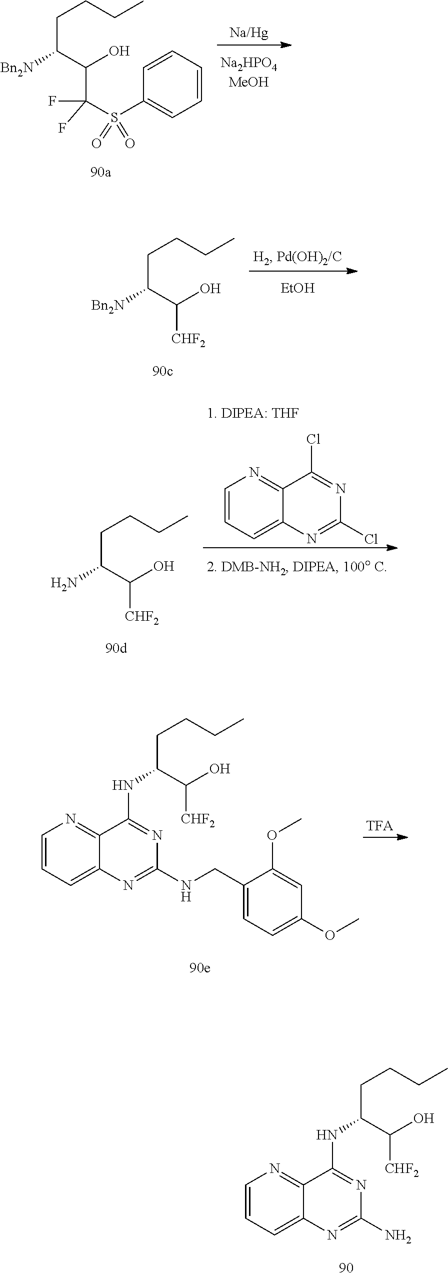

Synthesis of (2R,3R)-3-(dibenzylamino)-1,1-difluoro-1-(phenylsulfonyl)heptan-2-ol and (2S,3R)-3-(dibenzylamino)-1,1-difluoro-1-(phenylsulfonyl)heptan-2-ol (90a and 90b). A solution of compound 86c (235.6 mg, 0.798 mmol) and difluoromethyl phenyl sulfone (153.3 mg, 0.80 mmol) in THF (5 mL) was stirred at −78° C. and then 1.0 M LHMDS in THF (1.60 mL, 1.60 mmol) was added slowly. The reaction mixture was stirred for 2 h at −78° C., and warmed to rt. before quenching with saturated aqueous NH₄Cl solution (15 mL). The resulting solution was diluted with EtOAc (25 mL) and the two layers separated. The separated aqueous fraction was back extracted with EtOAc (15 mL×2). The separate organic fractions were washed with water (25 mL×2), brine (25 mL), then combined, dried over MgSO₄, filtered and concentrated in vacuo. The residue was subjected to silica gel chromatography eluting with 0-30% EtOAc in hexanes to afford of compound 90a as the first eluting isomer, and compound 90b as the second eluting isomer.

Compound 90a. LCMS-ESI⁺ (m/z): [M+H]⁺ calculated for C₂₇H₃₂F₂NO₃S: 488.21; found: 488.20; t_{R}=1.50 min on LC/MS Method A.

Compound 90b. LCMS-ESI⁺ (m/z): [M+H]⁺ calculated for C₂₇H₃₂F₂NO₃S: 488.21; found: 488.23; t_{R}=1.52 min on LC/MS Method A.

Synthesis of (3R)-3-(dibenzylamino)-1,1-difluoroheptan-2-ol (90c). To a solution of compound 90a (132.9 mg, 0.273 mmol) in methanol (2 mL) at −40° C. was added Na₂HPO₄ (236.3 mg, 1.664 mmol) and 5% sodium mercury-amalgam beads (646.1 mg, 1.41 mmol). The resulting mixture was stirred for 2 h in a cold bath, and then filtered through a Celite pad. The filtrate was concentrated in vacuo and the residue was treated with EtOAc (20 mL) and water (20 mL). The two layers were separated and the aqueous fraction was extracted with EtOAc (20 mL×2). The organic fractions were washed with water (20 mL×1), then combined, dried (MgSO₄), filtered and concentrated under reduced pressure. The residue was subjected to silica gel chromatography eluting with 0-30% EtOAc in hexanes to provide compound 90c. LCMS-ESI⁺ (m/z): [M+H]⁺ calculated for C₂₁H₂₈F₂NO: 348.21; found: 348.16; t_{R}=1.26 min on LC/MS Method A.

Synthesis of (3R)-3-amino-1,1-difluoroheptan-2-ol (90d). To a solution of compound 90c (27.2 mg, 0.078 mmol) in EtOH (1 mL) was added 20% palladium hydroxide on carbon (15.9 mg, 0.023 mmol). The resulting mixture was stirred under H₂ atmosphere for 20 h. The reaction mixture was then filtered and washed with EtOH (5 mL). The filtrate was concentrated in vacuo to obtain compound 90d. LCMS-ESI⁺ (m/z): [M+H]⁺ calculated for C₇H₁₁₆F₂NO: 168.12; found: 167.94; t_{R}=0.49 min on LC/MS Method A.

Synthesis of (3R)-3-((2-((2,4-dimethoxybenzyl)amino)pyrido[3,2-d]pyrimidin-4-yl)amino)-1,1-difluoroheptan-2-ol (90e). To a solution of compound 90d (12.4 mg, 0.074 mmol) and 2,4-dichloropyrido[3,2-d]pyrimidine (11.8 mg, 0.059 mmol) in THF (1 mL) was added N,N-diisopropylethylamine (0.039 mL, 0.222 mmol). The mixture was stirred for 2 h at rt, then additional THF (1 mL), N,N-diisopropylethylamine (0.039 mL, 0.222 mmol), and 2,4-dimethoxybenzylamine (0.056 mL, 0.371 mmol) were added, and the resulting mixture heated to 100° C. for 20 h. The reaction mixture was cooled to rt, diluted with EtOAc (˜20 mL), washed with water (20 mL×3) and brine (20 mL×1), dried (MgSO₄), filtered and concentrated in vacuo. The residue was subjected to silica gel chromatography eluting with 0-100% EtOAc in hexanes to isolate impure 90e. The impure material was then subjected to preparative HPLC purification (column, Gemini 10u C18110A, AXIA; 10% aq. acetonitrile-80% aq. acetonitrile with 0.1% TFA, over 20 min. gradient) to afford compound 90e LCMS-ESI⁺ (m/z): [M+H]⁺ calculated for C₂₃H₃₀F₂N₅O₃: 462.23; found: 462.17; t_{R}=1.00 min on LC/MS Method A.

Synthesis of (3R)-3-((2-aminopyrido[3,2-d]pyrimidin-4-yl)amino)-1,1-difluoroheptan-2-ol (90). Compound 90e (16 mg, 34.67 umol) was dissolved in TFA (1 mL) and stirred at rt. After 1 h, the mixture was concentrated in vacuo, and the residue was triturated in methanol (1 mL×3), filtered, and diluted with water (˜6 mL). The mixture was subjected to preparative HPLC (Gemini 10u C18110A, AXIA; 10% aq. acetonitrile-70% aq. acetonitrile with 0.1% TFA, over 20 min. gradient). Collected product fractions were concentrated in vacuo, co-evaporated with methanol (10 mL×3) and dried in vacuo to obtain compound 90 as its TFA salt. ¹H NMR (400 MHz, Methanol-d₄) δ 8.64 (dd, J=4.3, 1.4 Hz, 1H), 7.84 (dd, J=8.5, 1.5 Hz, 1H), 7.78 (dd, J=8.5, 4.3 Hz, 1H), 5.73 (td, J=55.6, 4.9 Hz, 1H), 4.70 (t, J=7.4 Hz, 1H), 3.98-3.82 (m, 1H), 1.90-1.72 (m, 2H), 1.54-1.31 (m, 4H), 1.00-0.82 (m, 3H). ¹⁹F NMR (376 MHz, Methanol-d₄) δ −77.78, −129.57 (ddd, J=289.8, 55.1, 8.6 Hz), −132.42 (ddd, J=290.1, 56.0, 12.5 Hz). LCMS-ESI⁺ (m/z): [M+H]⁺ calculated for C₁₄H₂₀F₂N₅O: 312.16; found: 312.15; t_{R}=0.74 min on LC/MS Method A.

### Example 91

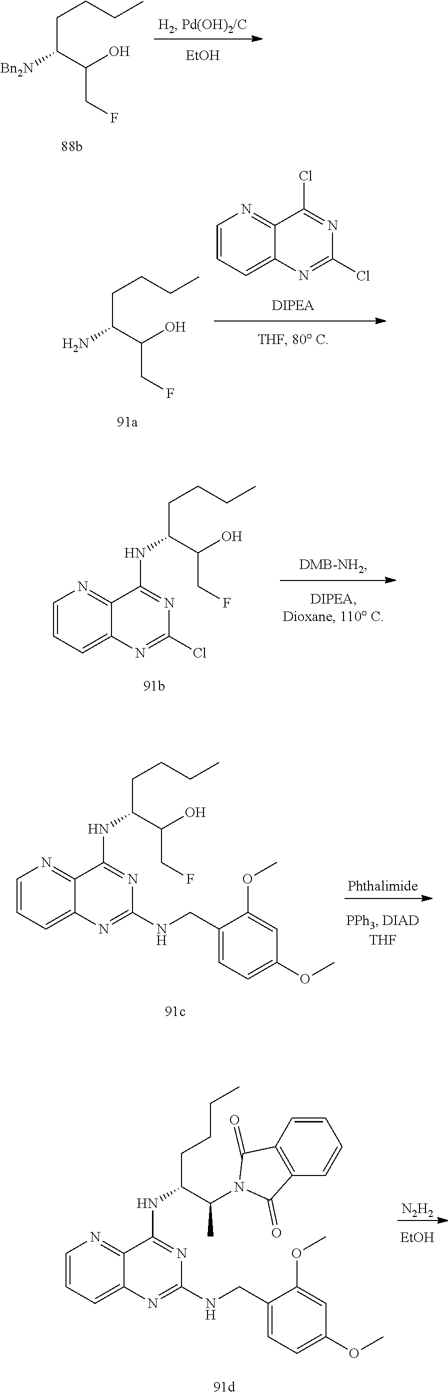

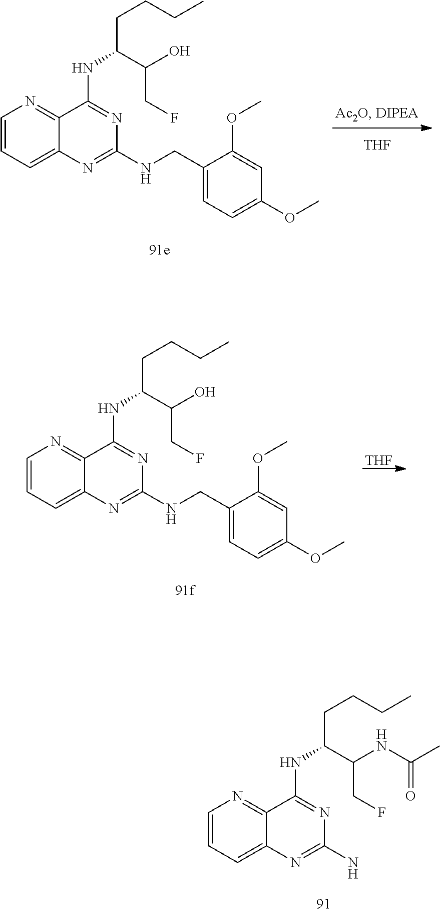 [SMILES: CCCC[C@@H](Nc1nc(NCc2ccc(OC)cc2OC)nc2cccnc12)C(O)CF ; CCCC[C@@H](Nc1nc(NCc2ccc(OC)cc2OC)nc2cccnc12)C(O)CF ; "THF" ; "THF" ; CCCC[C@@H](Nc1nc(N)nc2cccnc12)C(CF)NC(C)=O]

Synthesis of (3R)-3-amino-1-fluoroheptan-2-ol (91a). A mixture of compound 88b (300.1 mg, 0.911 mmol) and 20% palladium hydroxide on carbon (30.9 mg) in EtOH (5 mL) was stirred under H2. The reaction mixture was stirred for 20 h, filtered, and the solids were washed with EtOH (10 mL). The filtrate was concentrated in vacuo and the residue was co-evaporated with toluene (10 mL×2) to obtain compound 91a. LCMS-ESI⁺ (m/z): [M+H]⁺ calculated for C₇H₁₇FNO: 150.13; found: 149.95; t_{R}=0.47 min on LC/MS Method A.

Synthesis of (3R)-3-((2-chloropyrido[3,2-d]pyrimidin-4-yl)amino)-1-fluoroheptan-2-ol (91b). A solution of 91a (133.7 mg, 0.896 mmol) and 2,4-dichloropyrido[3,2-d]pyrimidine (201.6 mg, 1.008 mmol) in THF (6 mL) was treated with N,N-diisopropylethylamine (0.48 mL, 2.756 mmol). The mixture was stirred at rt for 2.75 h. The reaction mixture was concentrated in vacuo, and the residue was subjected to silica gel chromatography eluting with 20-70% EtOAc in hexanes to obtain, after removal of solvent in vacuo, compound 91b. LCMS-ESI⁺ (m/z): [M+H-C₂H₄]⁺ calculated for C₁₄H₁₉ClFN₄O: 313.12; found: 313.14; t_{R}=1.04 min on LC/MS Method A.

Synthesis of (3R)-3-((2-((2,4-dimethoxybenzyl)amino)pyrido[3,2-d]pyrimidin-4-yl)amino)-1-fluoroheptan-2-ol (91c). To a solution of compound 91b (233.6 mg, 0.747 mmol) in dioxane (7 mL) was added N,N-diisopropylethylamine (0.64 mL, 3.674 mmol), and 2,4-dimethoxybenzylamine (0.45 mL, 2.995 mmol). The resulting solution was refluxed at 110° C. bath for 24 h. The reaction mixture was concentrated in vacuo, and the residue was dissolved in DCM (30 mL), and washed with water (30 mL×1). The aqueous fraction was extracted with DCM (30 mL×1), and the organic fractions were combined, dried (MgSO₄), filtered and concentrated in vacuo. The residue was subjected to silica gel chromatography eluting with 20-100% EtOAc in hexanes. The collected fractions were concentrated under reduced pressure and the residue was subjected to preparative HPLC (Gemini 10u C18110A, AXIA; 10% aq. acetonitrile-80% aq. acetonitrile with 0.1% TFA, over 20 min. gradient). The collected product fractions were combined, neutralized by saturated aqueous NaHCO₃ solution (1 mL), partially concentrated in vacuo to remove acetonitrile and then extracted with EtOAc (20 mL×2). The organic extracts were washed with water (20 mL), combined, dried over MgSO₄, filtered and concentrated in vacuo to obtain compound 91c. LCMS-ESI⁺ (m/z): [M+H-C₂H₄]⁺ calculated for C₂₃H₃₁FN₅O₃: 444.24; found: 444.19; t_{R}=0.97 min on LC/MS Method A.

Synthesis of 2-((3R)-3-((2-((2,4-dimethoxybenzyl)amino)pyrido[3,2-d]pyrimidin-4-yl)amino)-1-fluoroheptan-2-yl)isoindoline-1,3-dione (91d). To a solution of compound 91c (654 mg, 1.475 mmol), phthalimide (347.1 mg, 2.359 mmol), and triphenylphosphine (874.8 mg, 3.359 mmol) in THF (24 mL) at 0° C. was added diisopropyl azodicarboxylate (0.697 mL, 3.539 mmol). The reaction mixture was warmed to rt and stirred for 2 h. After the reaction mixture was concentrated under reduced pressure, the residue was subjected to silica gel chromatography eluting with 0-100% EtOAc in hexanes to obtain, after removal of volatiles in vacuo, compound 91d. LCMS-ESI⁺ (m/z): [M+H]⁺ calculated for C₃₁H₃₄FN₆O₄: 573.26; found: 573.20; t_{R}=1.27 min on LC/MS Method A.

Synthesis of N⁴-((3R)-2-amino-1-fluoroheptan-3-yl)-N²-(2,4-dimethoxybenzyl)pyrido[3,2-d]pyrimidine-2,4-diamine (91e). To a solution of compound 91d (489.3 mg, 0.854 mmol) in EtOH (5 mL) was added hydrazine hydrate (0.07 mL, 1.28 mmol) at rt. The reaction mixture was refluxed for 3.5 h, the precipitates were removed by filtration and then the solid washed with EtOH (15 mL). The filtrates were concentrated in vacuo and the residue was dissolved in DCM (30 mL), washed with water (30 mL×2), dried over MgSO₄, filtered and concentrated in vacuo to obtain compound 91e. LCMS-ESI⁺ (m/z): [M+H]⁺ calculated for C₂₃H₃₂FN₆O₂: 443.26; found: 443.20; t_{R}=0.79 min on LC/MS Method A.

Synthesis of N-((3R)-3-((2-((2,4-dimethoxybenzyl)amino)pyrido[3,2-d]pyrimidin-4-yl)amino)-1-fluoroheptan-2-yl)acetamide (91f). To a solution of 91e (395.3 mg, 0.893 mmol) and N,N-diisopropylethylamine (0.311 mL, 1.787 mmol) in THF (8 mL) was added acetic anhydride (0.127 mL, 1.340 mmol), and the reaction was stirred for 30 min. at rt. The mixture was then diluted with EtOAc (30 mL), washed with saturated aqueous NaHCO₃ solution (30 mL), brine (30 mL), dried over MgSO₄, filtered and concentrated under reduced pressure. The residue was subjected to silica gel chromatography eluting with 0-100% EtOAc in hexanes, followed by elution with 0-20% methanol in EtOAc. The collected product fractions were concentrated in vacuo and then subjected to preparative HPLC purification (Gemini 10u C18110A, AXIA; 10% aq. acetonitrile-70% aq. acetonitrile with 0.1% TFA, over 20 min. gradient) to obtain, after removal of volatiles in vacuo, compound 91f. LCMS-ESI⁺ (m/z): [M+H]⁺ calculated for C₂₅H₃₄FN₆O₃: 485.27; found: 485.23; t_{R}=1.28 min on LC/MS Method A.

Synthesis of N-((3R)-3-((2-aminopyrido[3,2-d]pyrimidin-4-yl)amino)-1-fluoroheptan-2-yl)acetamide (91). Compound 91f (50 mg, 0.103 mmol) was dissolved in TFA (3 mL) and stirred at rt for 11 h. The mixture was concentrated under reduced pressure, and the residue was triturated with methanol (1 mL×3). After the insoluble material was removed by filtration and the filtrate was diluted with water (3 mL), the resulting solution was subjected to preparative HPLC (Gemini 10u C18110A, AXIA; 10% aq. acetonitrile-70% aq. acetonitrile with 0.1% TFA, over 20 min. gradient). Product-containing fractions were combined, concentrated under reduced pressure to dryness, co-evaporated with methanol (×3), and finally dried under high vacuum to provide 91 as its TFA salt. ¹H NMR (400 MHz, Methanol-d₄) δ 8.67 (ddd, J=4.3, 1.4, 0.6 Hz, 1H), 7.96-7.69 (m, 2H), 4.82-4.67 (m, 1H), 4.60 (d, J=5.1 Hz, 1H), 4.48 (d, J=5.0 Hz, 1H), 4.41 (dq, J=21.7, 5.1 Hz, 1H), 1.96 (d, J=4.2 Hz, 3H), 1.78 (td, J=8.6, 4.6 Hz, 1H), 1.48-1.24 (m, 4H), 0.90 (tt, J=5.5, 2.3 Hz, 3H). LCMS-ESI⁺ (m/z): [M+H]⁺ calculated for C₁₆H₂₇FN₆O: 335.19; found: 335.19; t_{R}=0.82 min on LC/MS Method A.

### Example 92

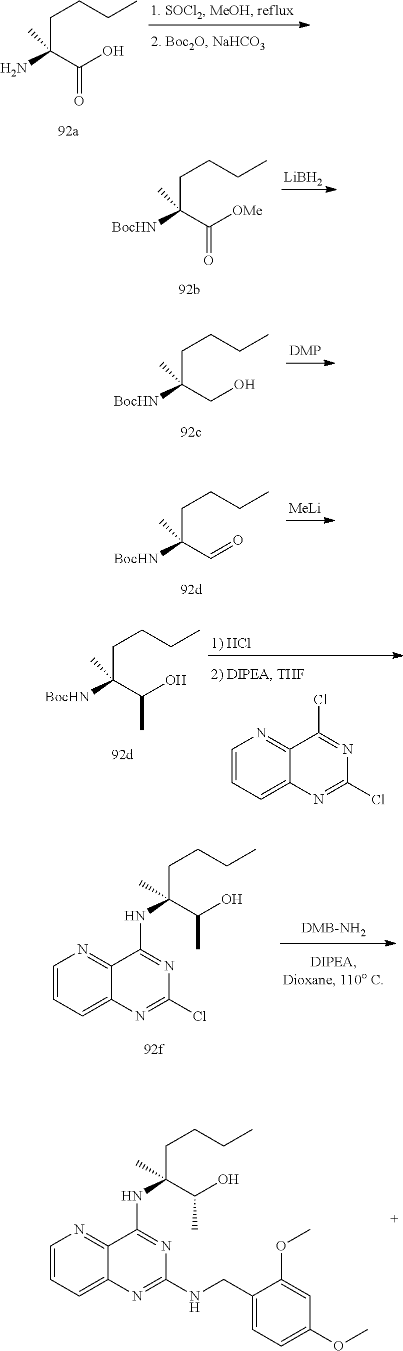

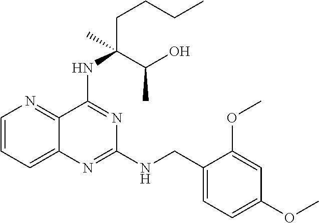 [SMILES: CCCC[C@](C)(Nc1nc(NCc2ccc(OC)cc2OC)nc2cccnc12)[C@H](C)O]

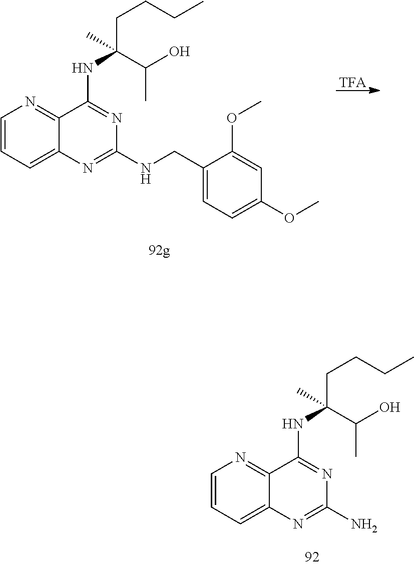 [SMILES: CCCC[C@](C)(Nc1nc(NCc2ccc(OC)cc2OC)nc2cccnc12)C(C)O ; CCCC[C@](C)(Nc1nc(N)nc2cccnc12)C(C)O ; "TFA"]

Synthesis of (S)-methyl 2-((tert-butoxycarbonyl)amino)-2-methylhexanoate (92b). To a suspension of (S)-2-amino-2-methylhexanoic acid 92a (2018.9 mg, 11.11 mmol, Asiba Pharmatech Inc.) in methanol (30 mL) was added thionyl chloride (1.62 mL) dropwise, and the resulting solution was refluxed for 41 h. The solution was concentrated under reduced pressure and the residue was co-evaporated with methanol (30 mL×2). The residue was treated with NaHCO₃ (4.6964 g, 55.90 mmol) in water (30 mL) and methanol (5 mL) and was stirred at rt. Di-tert-butyl dicarbonate (2932 mg, 13.43 mmol) was added and the mixture stirred for 4 h. Additional NaHCO₃ (1014.6 mg, 12.08 mmol) and di-tert-butyl dicarbonate (1234.0 mg, 5.654 mmol) were then added and the resulting suspension was stirred at rt overnight. The reaction mixture was then diluted with water (100 mL) and extracted with EtOAc (100 mL×2). The organic extracts were washed with water (100 mL), then combined, dried over MgSO₄ filtered and concentrated in vacuo. The residue was subjected to silica gel chromatography eluting with 0-20% EtOAc in hexanes to obtain compound 92b. LCMS-ESI⁺ (m/z): [M+H-C₄H₈]⁺ calculated for C₉H₁₈NO₄: 204.12; found: 203.68; t_{R}=1.24 min on LC/MS Method A.

Synthesis of (S)-tert-butyl (1-hydroxy-2-methylhexan-2-yl)carbamate (92c). To a stirred solution of compound 92b (2515.4 mg, 9.699 mmol) in THF (20 mL) and methanol (2.8 mL) at 0° C., was added 2.0 M LiBH₄ in THF (9.7 mL, 19.4 mmol). The solution was stirred at rt for 5 h, was and then diluted with water (100 mL) at 0° C., and extracted with EtOAc (100 mL×2). The combined extracts were washed with water (100 mL), dried over MgSO₄, filtered and concentrated in vacuo. The residue was subjected to silica gel chromatography eluting with 0-40% EtOAc in hexanes to provide compound 92c LCMS-ESI⁺ (m/z): [M+H-C₄H₈]⁺ calculated for C₁₂H₂₆NO₃: 232.19; found: 231.60; t_{R}=1.07 min on LC/MS Method A.

Synthesis of (S)-tert-butyl (2-methyl-1-oxohexan-2-yl)carbamate (92d). To a solution of compound 92c (543.3 mg, 2.349 mmol) in DCM (20 mL) was added Dess-Martin Periodinane (1495.1 mg, 3.525 mmol) and the resulting mixture stirred for 3 h. The reaction mixture was diluted with DCM (30 mL) and filtered through a pad of Celite. The filtrate was washed with saturated aqueous Na₂S₂O₃ (50 mL), water (50 mL), and brine (50 mL). The aqueous fraction was re-extracted with DCM (30 mL×2), and the combined organic fractions were dried over Na₂SO₄, filtered and concentrated in vacuo. The residue was subjected to silica gel chromatography eluting with 0-70% EtOAc in hexanes to obtain compound 92d. LCMS-ESI⁺ (m/z): [M+H-C₄H₈]⁺ calculated for C₈H₁₆NO₃: 174.11; found: 174.76, t_{R}=1.28 min on LC/MS Method A.

Synthesis of tert-butyl ((3S)-2-hydroxy-3-methylheptan-3-yl)carbamate (92e). To a solution of compound 92d (511.8 mg, 2.232 mmol) in diethyl ether (5 mL) cooled in an ice-salt bath (−15° C.), was added 1.6 M solution of MeLi in diethyl ether (5.58 mL, 8.927 mmol) dropwise over 5 min. After 30 min, the reaction mixture was quenched with saturated aqueous NH₄Cl solution (15 mL). The resulting mixture was diluted with water and the product was extracted with EtOAc (25 mL×2). The combined extracts were dried over MgSO₄, filtered and concentrated in vacuo. The residue was then subjected to silica gel chromatography eluting with 0-70% EtOAc in hexanes to provide compound 92e as a mixture of two diastereomers. LCMS-ESI⁺ (m/z): [M+H]⁺ calculated for C₁₃H₂₈NO₃: 246.21; found: 245.63; t_{R}=1.28 min on LC/MS Method A.

Synthesis of (3S)-3-((2-chloropyrido[3,2-d]pyrimidin-4-yl)amino)-3-methylheptan-2-ol (92f). Compound 92e (347 mg, 1.414 mmol) was dissolved in 4M HCl in dioxane (3.1 mL) and stirred at rt for 4 h. The reaction mixture was then concentrated in vacuo. The residue in THF (10.5 mL) was treated with 2,4-dichloropyrido[3,2-d]pyrimidine (259.1 mg, 1.295 mmol) and N,N-diisopropylethylamine (1.18 mL, 6.77 mmol), and placed in 80° C. bath for 1 h. The reaction mixture was cooled to rt, concentrated under reduced pressure, and the residue subjected to silica gel chromatography eluting with 0-70% EtOAc in hexanes to obtain compound 92f. LCMS-ESI⁺ (m/z): [M+H]⁺ calculated for C₁₅H₂₁ClN₄O: 309.15; found: 309.12; t_{R}=1.32 min on LC/MS Method A.

Synthesis of (2R,3S)-3-((2-((2,4-dimethoxybenzyl)amino)pyrido[3,2-d]pyrimidin-4-yl)amino)-3-methylheptan-2-ol and (2S,3S)-3-((2-((2,4-dimethoxybenzyl)amino)pyrido[3,2-d]pyrimidin-4-yl)amino)-3-methylheptan-2-ol (92g and 92h). To a solution of compound 92f (331.8 mg, 1.074 mmol) in dioxane (11 mL) was added N,N-diisopropylethylamine (0.561 mL, 3.223 mmol) and 2,4-dimethoxybenzylamine (0.807 mL, 5.372 mmol). The resulting mixture was refluxed at 110° C. bath for 17 h. The mixture was then concentrated in vacuo and the resulting residue dissolved in EtOAc (50 mL) and washed with water (50 mL×2) and brine (50 mL). The organic fraction was dried over Na₂SO₄, filtered and then concentrated in vacuo. The resulting residue was subjected to silica gel chromatography eluting with 0-100% EtOAc in hexanes. The collected product was then concentrated in vacuo and resubjected to column chromatography on silica gel eluting with 0-20% MeOH in DCM to obtain a mixture of compound 92g and 92h. The mixture was then concentrated in vacuo and the residue subjected to preparative chiral SFC (SFC IC-5 um-4.6×100 mm, 40% EtOH-ammonia) to obtain after removal of volatiles in vacuo compound 92g eluting first, and compound 92h eluting second.

Compound 92g: ¹H NMR (400 MHz, Chloroform-d) δ 8.29 (dd, J=4.5, 1.5 Hz, 1H), 7.71 (d, J=8.4 Hz, 1H), 7.44 (dd, J=8.5, 4.3 Hz, 1H), 7.29 (d, J=8.2 Hz, 1H), 6.46 (d, J=2.4 Hz, 1H), 6.42 (dd, J=8.2, 2.4 Hz, 1H), 4.56 (d, J=5.8 Hz, 2H), 3.84 (s, 3H), 3.79 (s, 3H), 2.13 (t, J=12.7 Hz, 1H), 1.88 (t, J=11.5 Hz, 1H), 1.45 (ddd, J=12.9, 9.7, 5.5 Hz, 1H), 1.38 (s, 3H), 1.35-1.22 (m, 2H), 1.21 (d, J=6.3 Hz, 4H), 0.87 (t, J=7.2 Hz, 3H). LCMS-ESI⁺ (m/z): [M+H]⁺ calculated for C₂₄H₃₄N₅O₃: 440.27; found: 440.18; t_{R}=1.29 min on LC/MS Method A.

Compound 92h: ¹H NMR (400 MHz, Chloroform-d) δ 8.29 (dd, J=4.3, 1.5 Hz, 1H), 7.70 (d, J=8.4 Hz, 1H), 7.43 (dd, J=8.5, 4.3 Hz, 1H), 7.29 (d, J=8.2 Hz, 1H), 7.20 (s, 1H), 6.46 (d, J=2.4 Hz, 1H), 6.42 (dd, J=8.2, 2.4 Hz, 1H), 4.56 (d, J=5.7 Hz, 2H), 3.84 (s, 3H), 3.79 (s, 3H), 1.97 (d, J=10.6 Hz, 1H), 1.59 (dt, J=13.9, 7.2 Hz, 1H), 1.48 (s, 3H), 1.36 (qd, J=7.2, 6.7, 4.0 Hz, 4H), 1.26 (d, J=1.4 Hz, 1H), 1.18 (d, J=6.4 Hz, 3H), 0.97-0.90 (m, 3H). LCMS-ESI⁺ (m/z): [M+H]⁺ calculated for C₂₄H₃₄N₅O₃: 440.27; found: 440.18; t_{R}=1.28 min on LC/MS Method A.

Synthesis of (3S)-3-((2-aminopyrido[3,2-d]pyrimidin-4-yl)amino)-3-methylheptan-2-ol (92). Compound 92g (74.1 mg, 0.169 mmol) was dissolved in TFA (3 mL) and stirred at rt for 0.75 h. The reaction mixture was carefully concentrated under reduced pressure to dryness. The residue was triturated with 50% aq. methanol and filtered through a Celite-membrane filter. The filtrate was then subjected to preparative HPLC (Gemini 10u C18110A, AXIA; 10% aq. acetonitrile-70% aq. acetonitrile with 0.1% TFA, over 20 min. gradient). The product fractions were combined, concentrated in vacuo, then co-evaporated with methanol (10 mL×3), and dried under vacuum to provide compound 92 as its TFA salt. ¹H NMR (400 MHz, Methanol-d₄) δ 8.61 (dd, J=4.4, 1.4 Hz, 1H), 7.84 (dd, J=8.5, 1.4 Hz, 1H), 7.76 (dd, J=8.5, 4.4 Hz, 1H), 4.36 (q, J=6.5 Hz, 1H), 2.30 (dt, J=16.4, 6.8 Hz, 1H), 1.91-1.78 (m, 1H), 1.56 (s, 3H), 1.46-1.29 (m, 4H), 1.23 (d, J=6.5 Hz, 3H), 0.97-0.85 (m, 3H). ¹⁹F NMR (376 MHz, Methanol-d₄) δ −77.60. LCMS-ESI⁺ (m/z): [M+H]⁺ calculated for C₁₅H₂₄N₅O: 290.20; found: 290.14; t_{R}=0.82 min on LC/MS Method A.

### Example 93

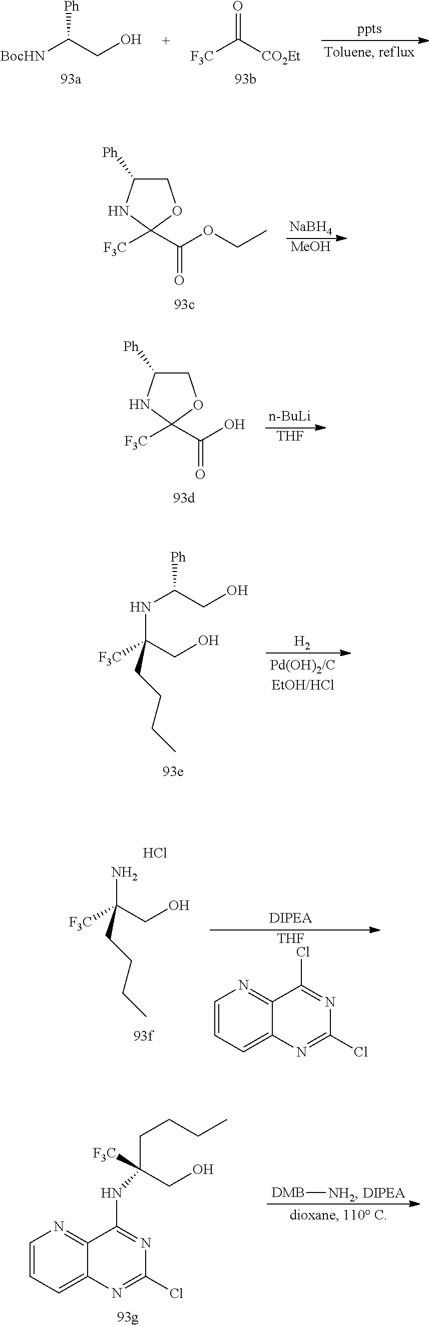

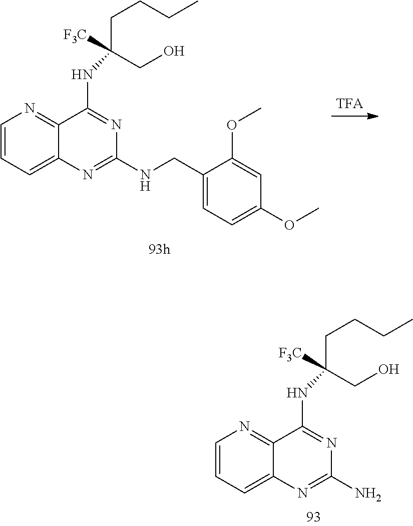 [SMILES: CCCC[C@](CO)(Nc1nc(NCc2ccc(OC)cc2OC)nc2cccnc12)C(F)(F)F ; CCCC[C@](CO)(Nc1nc(N)nc2cccnc12)C(F)(F)F]

Synthesis of (4R)-ethyl 4-phenyl-2-(trifluoromethyl)oxazolidine-2-carboxylate (93c). A solution of (R)—N-Boc-phenylglycinol 93a (522.4 mg, 2.249 mmol, Combi-Blocks, Inc.), ethyl trifluoropyruvate 93b (0.328 mL, 2.474 mmol, Oakwood Products), and pyridinium p-toluenesulfonate (113.1 mg, 0.450 mmol) in toluene (20 mL) was refluxed with a Dean-Stark apparatus for 20 h. The reaction mixture was then cooled to 0° C. using an ice-water bath and filtered through a pad of Celite. After the filtrate was concentrated in vacuo, the residue was subjected to silica gel chromatography eluting with 0-30% EtOAc in hexanes to obtain compound 93c. LCMS-ESI⁺ (m/z): [M+H]⁺ calculated for C₁₃H₁₅F₃NO₃: 290.10; found: 289.84; t_{R}=1.21 min on LC/MS Method A.

Synthesis of ((4R)-4-phenyl-2-(trifluoromethyl)oxazolidin-2-yl)methanol (93d). To a solution of compound 93c (384.9 mg, 1.331 mmol) in MeOH (6 ML) at 0° C. was added sodium borohydride (50.3 mg, 1.331 mmol). The reaction mixture was warmed to rt and stirred for 30 min. before quenching with aqueous saturated NH₄Cl (15 mL). After methanol was removed under reduced pressure, the resulting aqueous solution was extracted with EtOAc (25 mL×3). The organic extracts were washed with water (25 mL×2) and brine (25 mL), combined, dried over MgSO₄, filtered and then concentrated in vacuo. The residue was subjected to silica gel chromatography eluting with 0-40% EtOAc in hexanes to obtain compound 93d LCMS-ESI⁺ (m/z): [M+H]⁺ calculated for C₁₁H₁₃F₃NO₂: 248.09; found: 247.90; t_{R}=0.96 min on LC/MS Method A.

Synthesis of (R)-2-(((R)-2-hydroxy-1-phenylethyl)amino)-2-(trifluoromethyl)hexan-1-ol (93e). To a solution of compound 93d (264.7 mg, 1.071 mmol) in THF (13 mL) at −78° C. was added n-butyllithium (2.5 M in hexane, 1.713 mL, 4.283 mmol) dropwise. The resulting solution was stirred in a cold bath for 2 h before quenching with aqueous saturated NH₄Cl (30 mL). The mixture was extracted with EtOAc (30 mL×3) and the extracts were washed with water (30 mL×2) and brine (30 mL×1). The organic fractions were combined, dried over MgSO₄, filtered and concentrated in vacuo. The residue was subjected to silica gel chromatography eluting with 0-70% EtOAc in hexanes to obtain compound 93e LCMS-ESI⁺ (m/z): [M+H]⁺ calculated for C₁₅H₂₃F₃NO₂: 306.17; found: 305.90, t_{R}=1.13 min on LC/MS Method A.

Synthesis of (R)-2-amino-2-(trifluoromethyl)hexan-1-ol hydrochloride (93f). To a solution of compound 93e (146.5 mg, 0.480 mmol) in EtOH (1 mL) and concentrated HCl (0.3 mL) was added palladium hydroxide on carbon (67.4 mg) and the resulting mixture was stirred under H₂ atmosphere for 24 h. The reaction mixture was filtered through a pad of Celite and then the solids rinsed with EtOH (25 mL). The eluants were concentrated under reduced pressure, diluted with water (20 mL) and then extracted with EtOAc (20 mL×2). The organic extracts were combined and concentrated under reduced pressure to obtain of compound 93f as its HCl salt. LCMS-ESI⁺ (m/z): [M+H]⁺ calculated for C₇H₁₅F₃NO: 186.11; found: 185.95; t_{R}=0.51 min on LC/MS Method A.

Synthesis of (R)-2-((2-((2,4-dimethoxybenzyl)amino)pyrido[3,2-d]pyrimidin-4-yl)amino)-2-(trifluoromethyl)hexan-1-ol (93h). To a solution of compound 93f (123.84 mg, 0.480 mmol) and 2,4-dichloropyrido[3,2-d]pyrimidine (96.0 mg, 0.480 mmol) in THF (4 mL) was added N,N-diisopropylethylamine (0.251 mL, 1.439 mmol). The reaction mixture was stirred and heated to 80° C. for 18 h. The reaction mixture was allowed to cool and concentrated in vacuo. The resulting residue was subjected to silica gel chromatography eluting with 0-100% EtOAc in hexanes to afford compound 93g (109.9 mg, 66%). To a solution of compound 93g (109.9 mg, 0.315 mmol) in dioxane (3.5 mL) was added N,N-diisopropylethylamine (0.165 mL, 0.945 mmol) and 2,4-dimethoxybenzylamine (0.237 mL, 1.576 mmol). The mixture was refluxed at 110° C. for 20 h, allowed to cool to rt, diluted with EtOAc (30 mL), washed with water (30 mL×3) and brine (30 mL), dried over MgSO₄, filtered and concentrated in vacuo. The resulting residue was subjected to silica gel chromatography eluting with 0-100% EtOAc in hexanes. The collected fractions were concentrated in vacuo to a residue that was subjected to preparative HPLC purification (Gemini 10u C18110A, AXIA; 10% aq. acetonitrile-80% aq. acetonitrile with 0.1% TFA, over 20 min. gradient) to afford compound 93h LCMS-ESI⁺ (m/z): [M+H]⁺ calculated for C₂₃H₂₉F₃N₅O₃: 480.22; found: 480.17; t_{R}=0.96 min on LC/MS Method A.

Synthesis of (R)-2-((2-aminopyrido[3,2-d]pyrimidin-4-yl)amino)-2-(trifluoromethyl)hexan-1-ol (93). Compound 93h (7.8 mg, 16.27 umol) was dissolved in TFA (1 mL) and stirred at rt for 1 h. The reaction mixture was then concentrated in vacuo and the residue was co-evaporated with methanol (5 mL×3). The residue was triturated with 50% aq. methanol and filtered through a Celite-membrane filter. The filtrate was subjected to preparative HPLC (Gemini 10u C18110A, AXIA; 10% aq. acetonitrile-70% aq. acetonitrile with 0.1% TFA, over 20 min. gradient). The product fractions were combined, concentrated under reduced pressure, co-evaporated with methanol (10 mL×3), and dried under vacuum to provide compound 93 as its TFA salt. ¹H NMR (400 MHz, Methanol-d₄) δ 8.67 (dd, J=4.4, 1.4 Hz, 1H), 7.89 (dd, J=8.5, 1.4 Hz, 1H), 7.82 (dd, J=8.5, 4.4 Hz, 1H), 4.11 (d, J=12.2 Hz, 1H), 4.06-3.97 (m, 1H), 2.81 (ddd, J=13.8, 11.0, 4.4 Hz, 1H), 1.99-1.85 (m, 1H), 1.38 (m, 4H), 0.92 (t, J=7.0 Hz, 3H). ¹⁹F NMR (376 MHz, Methanol-d₄) δ −75.96 (s, 3F), −77.39 (s, 3F). LCMS-ESI⁺ (m/z): [M+H]⁺ calculated for C₁₄H₁₉F₃N₅O: 330.15; found: 330.16; t_{R}=0.76 min on LC/MS Method A.

### Example 94

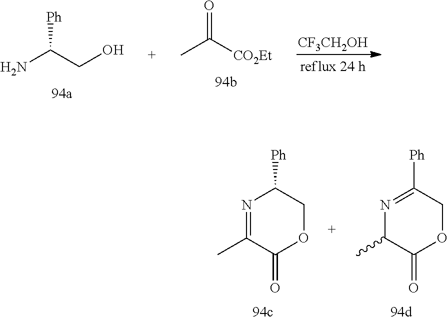

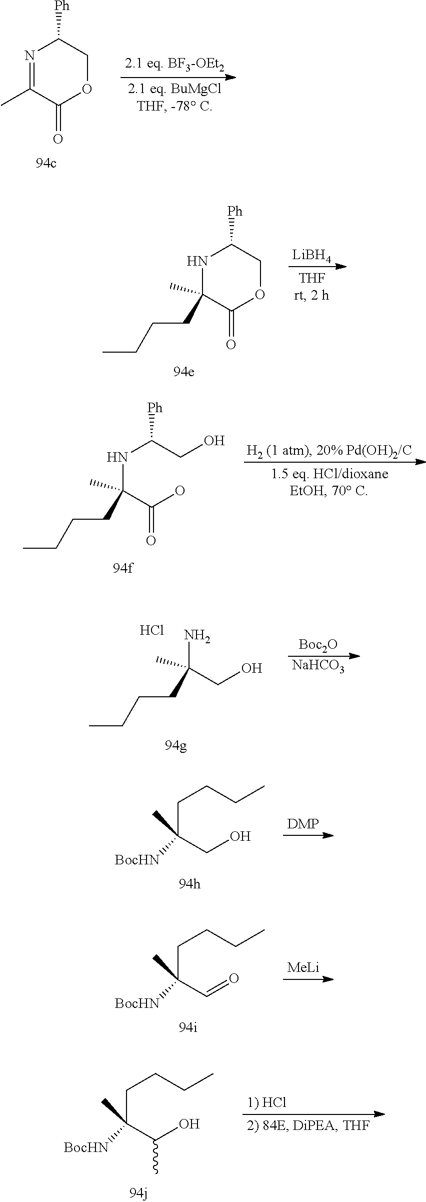

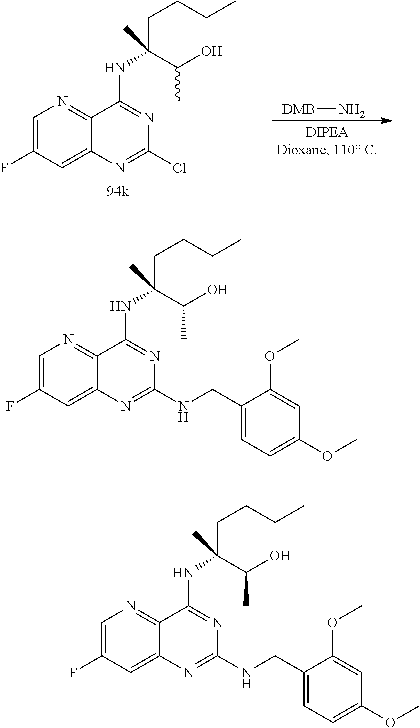

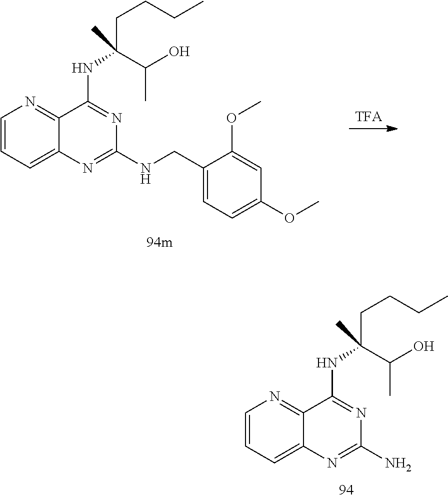 [SMILES: CCCC[C@@](C)(Nc1nc(NCc2ccc(OC)cc2OC)nc2cccnc12)C(C)O ; CCCC[C@@](C)(Nc1nc(N)nc2cccnc12)C(C)O]

Synthesis of (R)-3-methyl-5-phenyl-5,6-dihydro-2H-1,4-oxazin-2-one (94c) and 3-methyl-5-phenyl-3,6-dihydro-2H-1,4-oxazin-2-one (94d). To a mixture of (R)-(−)-2-phenylglycinol 94a, (Sigma-Aldrich, 98%, 99% ee, 3.6296 g, 172.25 mmol) and molecular sieves (86.03 g) in 2,2,2-trifluoroethanol (500 mL) was added ethyl pyruvate 94b (19.2 mL, 172.29 mmol) and the resulting mixture heated to reflux temperature. After 24 h, the mixture was cooled to rt, filtered through a pad of Celite, and washed with EtOAc (50 mL). The orange filtrate and the EtOAc washes were separated into two flasks and each was concentrated under reduced pressure. Each of the resulting residues was subjected to silica gel chromatography eluting with 0-40% EtOAc in hexanes. Product fractions from the two chromatographies were combined, concentrated under reduced pressure, and dried in vacuo to provide compound 94c as well as the later eluting compound 94d.

Compound 94c: ¹H NMR (400 MHz, Chloroform-d) δ 7.45-7.38 (m, 2H), 7.38-7.32 (m, 3H), 4.85 (ddd, J=10.9, 4.6, 2.4 Hz, 1H), 4.57 (dd, J=11.6, 4.5 Hz, 1H), 4.26 (dd, J=11.6, 10.9 Hz, 1H), 2.41 (d, J=2.4 Hz, 3H). LCMS-ESI⁺ (m/z): [M+H]⁺ calculated for C₁₁H₁₂NO₂: 190.09; found: 189.92; t_{R}=0.88 min on LC/MS Method A.

Compound 94d: ¹H NMR (400 MHz, Chloroform-d) δ 7.81-7.71 (m, 2H), 7.55-7.41 (m, 3H), 5.47 (dd, J=16.0, 1.2 Hz, 1H), 5.25 (dd, J=16.0, 2.8 Hz, 1H), 4.31 (qdd, J=7.1, 3.0, 1.1 Hz, 1H), 1.72 (d, J=7.3 Hz, 3H). LCMS-ESI⁺ (m/z): [M+H]⁺ calculated for C₁₁H₁₂NO₂: 190.09; found: 189.94; t_{R}=0.83 min on LC/MS Method A.

Synthesis of (3R,5R)-3-butyl-3-methyl-5-phenylmorpholin-2-one (94e). A solution of compound 94c (14.84 g, 78.43 mmol) in THF (500 mL) was stirred at −78° C. bath under argon and boron trifluoride diethyl etherate (20.5 mL, 161.11 mmol) was added slowly over 30 min. The reaction mixture was allowed to stir at −78° C. for 1.5 h. 2M butylmagnesium chloride solution 2.0 M in THF (83.0 mL) was added slowly over ˜30 min. and the reaction mixture was allowed to stir at −78° C. for 2h before addition of saturated ammonium chloride (300 mL) followed by warming to rt. The mixture was diluted with water (200 mL) and extracted with EtOAc (300 mL×3). The organic extracts were washed with water (500 mL×3), brine (300 mL), combined, dried (Na₂SO₄), and concentrated under reduced pressure. After the residue was dissolved in DCM (150 mL, heating), the insoluble material was removed by filtration. The filtrate was concentrated under reduced pressure to a small volume, and was subjected to silica gel chromatography eluting eluting with 0-20% EtOAc in hexanes to provide compound 94e. LCMS-ESI⁺ (m/z): [M+H]⁺ calculated for C₁₅H₂₂NO₂: 248.17; found: 248.02; t_{R}=1.07 min on LC/MS Method A.

Synthesis of (R)-2-(((R)-2-hydroxy-1-phenylethyl)amino)-2-methylhexan-1-ol (94f). To a stirred solution of compound 94e (14.01 g, 56.64 mmol) in THF (100 mL) at 0° C. was added 2.0 M LiBH₄ in THF (57 mL, 114 mmol). The solution was stirred at rt for 2 h, cooled with an ice bath and quenched with water (500 mL). The product was extracted with EtOAc (300 mL×3) and the extracts were washed with water (500 mL) and brine (100 mL). The combined extracts were dried (Na₂SO₄) and concentrated under reduced pressure to obtain 94f LCMS-ESI⁺ (m/z): [M+H]⁺ calculated for C₁₅H₂₆NO₂: 252.20; found: 252.05; t_{R}=0.68 min on LC/MS Method A.

Synthesis of (R)-2-amino-2-methylhexan-1-ol hydrochloride (94g). To a mixture of compound 94f (14.24 g, 56.65 mmol) and 20% Pd(OH)₂ on carbon (2.847 g) in EtOH (210 mL) was added 4 N HCl in dioxane (21.5 mL, 86.0 mmol) The resulting mixture was purged with H₂ gas (3 times) and then stirred under H₂ atmosphere at 70° C. for 8 h. The reaction mixture was allowed to cool and additional 20% Pd(OH)₂ on carbon (0.71 g) was added. The resulting mixture was purged with H₂ gas (3 times) and then stirred under H₂ atmosphere at 70° C. for 2 h. The reaction mixture was cooled and filtered through a Celite pad and the removed solids washed with EtOH (50 mL). The filtrate and EtOH washings were combined and concentrated under reduced pressure. The residue was co-evaporated with DCM (100 mL×3) and dried under vacuum to give compound 94g. The residue was triturated with DCM (50 mL) and toluene (50 mL) and then concentrated under reduced pressure. The residue was co-evaporated with toluene (50 mL×1) and dried under vacuum at 40° C. for 1 h, and rt overnight to obtain compound 94g as its HCl salt. LCMS-ESI⁺ (m/z): [M+H]⁺ calculated for C₇H₁₈NO: 132.14; found: 131.90; t_{R}=0.42 min on LC/MS Method A.

Synthesis of (R)-tert-butyl (1-hydroxy-2-methylhexan-2-yl)carbamate (94h). To a solution of 94g (3.1403 g, 16.01 mmol) in methanol (7 mL) and water (45 mL) was added sodium bicarbonate (4.05 g, 48.21 mmol) and di-tert-butyl dicarbonate (Boc₂O, 4.25 g, 19.47 mmol). The resulting mixture was stirred at rt for 3 h and then additional sodium bicarbonate (0.68 g, 8.095 mmol) and di-tert-butyl dicarbonate (1.752 g, 8.028 mmol) were added. The mixture was stirred for 48 h and then additional sodium bicarbonate (0.808 g, 9.618 mmol) and di-tert-butyl dicarbonate (1.92 g, 8.797 mmol) were added. The reaction mixture was stirred for 4 h, diluted with water (100 mL), and extracted with EtOAc (100 mL×2). The extracts were washed with water (100 mL), dried over MgSO₄, filtered and then concentrated under reduced pressure. The residue was subjected to silica gel chromatography eluting with 0-40% EtOAc in hexanes to obtain compound 94h LCMS-ESI⁺ (m/z): [M+H]⁺ calculated for C₁₂H₂₆NO₃: 232.19; found: 231.65; t_{R}=1.08 min on LC/MS Method A.

Synthesis of (R)-tert-butyl (2-methyl-1-oxohexan-2-yl)carbamate (94i). To a solution of compound 94h (446.7 mg, 1.931 mmol) in DCM (15 mL) was added Dess-Martin Periodinane (1230.6 mg, 2.901 mmol) and the resulting mixture was stirred for 3 h. The reaction mixture was filtered through a pad of Celite, and the filtrate was then washed with saturated aqueous Na₂S₂O₃ (30 mL) followed by water (30 mL×2). The aqueous fractions were back extracted with DCM (30 mL), and all the organic fractions were then combined, dried over MgSO₄, filtered and concentrated in vacuo. The resulting residue was subjected to silica gel chromatography eluting with 0-30% EtOAc in hexanes to obtain compound 94i. LCMS-ESI⁺ (m/z): [M+H-C₄H₈]⁺ calculated for C₈H₁₆NO₃: 174.11; found: 173.77; t_{R}=1.17 min on LC/MS Method A.

Synthesis of tert-butyl ((3R)-2-hydroxy-3-methylheptan-3-yl)carbamate (94j). To a solution of compound 94i (322.4 mg, 1.406 mmol) in diethyl ether (5 mL) in an ice-NaCl bath was added 1.6 M MeLi in diethyl ether (3.6 mL, 5.76 mmol) dropwise over 2 min. After 30 min, the reaction mixture was quenched with saturated aqueous ammonium chloride solution (20 mL). The two phases were separated and the aqueous fraction was extracted with DCM (30 mL). The organic fractions were washed with water (30 mL), combined, dried over MgSO₄, filtered and then concentrated in vacuo. The residue was then subjected to silica gel chromatography eluting with 0-40% EtOAc in hexanes to obtain compound 94j. LCMS-ESI⁺ (m/z): [M+H]⁺ calculated for C₁₃H₂₈NO₃: 246.21; found: 245.70; t_{R}=1.14 min. and t_{R}=1.16 min on LC/MS Method A.

Synthesis of (3R)-3-((2-chloro-7-fluoropyrido[3,2-d]pyrimidin-4-yl)amino)-3-methylheptan-2-ol (94k). Compound 94j (119.8 mg, 0.488 mmol) was dissolved in 4M HCl in dioxane (3 mL) and stirred at rt for 1 h. The reaction mixture was concentrated in vacuo and the residue was then treated with THF (10.5 mL) followed by 2,4-dichloro-7-fluoropyrido[3,2-d]pyrimidine 84E (110.9 mg, 0.508 mmol) and N,N-diisopropylethylamine (0.36 mL, 2.067 mmol). The mixture was heated in a 80° C. bath for 3 h. The reaction mixture was allowed to cool to rt, concentrated in vacuo and the residue subjected to silica gel chromatography eluting with 0-100% EtOAc in hexanes to obtain compound 94k as a mixture of two diastereomers (˜2:3 ratio). ¹H NMR (400 MHz, Chloroform-d) δ 8.55 (dd, J=2.6, 1.2 Hz, 1H), 7.66 (dd, J=8.8, 2.6 Hz, 1H), 7.35 (d, J=10.9 Hz, 1H), 5.29 (br, 1H), 3.97 (q, J=6.1 Hz, 0.4H), 3.91 (q, J=6.4 Hz, 0.6H), 2.09 (ddd, J=13.8, 12.3, 4.4 Hz, 0.6H), 2.03-1.88 (m, 1H), 1.67 (dt, J=14.2, 7.0 Hz, 0.4H), 1.51 (s, 1.2H), 1.43 (s, 1.8H), 1.49-1.136 (m, 4H), 1.22 (d, J=6.5 Hz, 1.8H), 1.20 (d, J=6.5 Hz, 1.2H), 0.99-0.91 (m, 1.2H), 0.88 (t, J=7.3 Hz, 1.8H). ¹⁹F NMR (376 MHz, Chloroform-d) δ −117.38 (t, J=8.9 Hz). LCMS-ESI⁺ (m/z): [M+H]⁺ calculated for C₁₅H₂₁ClFN₄O: 327.14; found: 327.11; t_{R}=1.23 min on LC/MS Method A.

Synthesis of (2R,3R)-3-((2-((2,4-dimethoxybenzyl)amino)-7-fluoropyrido[3,2-d]pyrimidin-4-yl)amino)-3-methylheptan-2-ol and (2S,3R)-3-((2-((2,4-dimethoxybenzyl)amino)-7-fluoropyrido[3,2-d]pyrimidin-4-yl)amino)-3-methylheptan-2-ol (94l and 94m). To a solution of compound 94k (128.5 mg, 0.416 mmol) in dioxane (5 mL) was added N,N-diisopropylethylamine (0.22 mL, 1.263 mmol) and 2,4-dimethoxybenzylamine (0.16 mL, 1.065 mmol) and the resulting mixture was refluxed in a 110° C. bath for 20 h. The reaction mixture was allowed to cool to rt, diluted with EtOAc (30 mL) and then washed with water (30 mL×2). The aqueous fractions were then back extracted with EtOAc (30 mL). The organic fractions were combined, dried over MgSO₄, and concentrated under reduced pressure. The residue was then subjected to silica gel chromatography eluting with 0-100% EtOAc in hexanes to obtain a mixture of compounds 94l and 94m. The compound mixture was further subjected to preparative chiral SFC (SFC IC-5 um-4.6×100 mm, 30% EtOH-ammonia, flow rate=3 mL/min) to obtain, compound 94l, eluting first, and compound 94m, eluting second.

Compound 94l: ¹H NMR (400 MHz, Chloroform-d) δ 8.14 (d, J=2.5 Hz, 1H), 7.32 (s, 1H), 7.28 (d, J=8.3 Hz, 1H), 6.46 (d, J=2.4 Hz, 1H), 6.42 (dd, J=8.3, 2.4 Hz, 1H), 4.55 (d, J=5.7 Hz, 2H), 3.84 (s, 3H), 3.79 (s, 3H), 4.0-3.7 (m, 1H), 1.97 (s, 1H), 1.59 (s, 2H), 1.47 (s, 3H), 1.36 (d, J=5.2 Hz, 4H), 1.17 (d, J=6.4 Hz, 3H), 1.00-0.89 (m, 3H). ¹⁹F NMR (376 MHz, Chloroform-d) δ −121.41. LCMS-ESI⁺ (m/z): [M+H]⁺ calculated for C₂₄H₃₃FN₅O₃: 458.26; found: 458.17; t_{R}=1.19 min on LC/MS Method A.

Compound 94m: ¹H NMR (400 MHz, Chloroform-d) δ 8.14 (d, J=2.6 Hz, 1H), 7.33 (s, 1H), 7.28 (d, J=8.3 Hz, 1H), 6.46 (d, J=2.3 Hz, 1H), 6.42 (dd, J=8.3, 2.4 Hz, 1H), 4.55 (d, J=5.8 Hz, 2H), 3.84 (d, J=1.1 Hz, 3H), 3.79 (s, 3H), 3.9-3.6 (m, 1H), 2.09 (d, J=14.1 Hz, 1H), 1.87 (s, 1H), 1.57 (s, 1H), 1.43 (m, 1H), 1.37 (s, 3H), 1.30 (m, 2H), 1.20 (d, J=6.4 Hz, 3H), 0.87 (t, J=7.2 Hz, 3H). ¹⁹F NMR (376 MHz, Chloroform-d) δ −121.40. LCMS-ESI⁺ (m/z): [M+H]⁺ calculated for C₂₄H₃₃FN₅O₃: 458.26; found: 458.16; t_{R}=1.22 min on LC/MS Method A.

Synthesis of (3R)-3-((2-amino-7-fluoropyrido[3,2-d]pyrimidin-4-yl)amino)-3-methylheptan-2-ol (94). Compound 94m (9.0 mg, 20.5 umol) was dissolved in TFA (1 mL) and stirred at rt for 1 h. The reaction mixture was carefully concentrated under reduced pressure to dryness, and the residue was then triturated with 50% aq. methanol, and filtered through a Celite-membrane filter. The filtrate was subjected to preparative HPLC (Gemini 10u C18110A, AXIA; 10% aq. acetonitrile-70% aq. acetonitrile with 0.1% TFA, over 20 min. gradient). The product fractions were combined, concentrated under reduced pressure, co-evaporated with methanol (10 mL×3), and dried under vacuum to obtain compound 94 as its TFA salt. ¹H NMR (400 MHz, Methanol-d₄) δ 8.54 (d, J=2.4 Hz, 1H), 8.31 (s, 1H), 7.62 (dd, J=8.8, 2.5 Hz, 1H), 4.39-4.29 (m, 1H), 2.29 (dt, J=15.7, 6.7 Hz, 1H), 1.84 (dt, J=16.0, 6.9 Hz, 1H), 1.55 (s, 3H), 1.44-1.30 (m, 4H), 1.23 (d, J=6.5 Hz, 3H), 0.96-0.84 (m, 3H). ¹⁹F NMR (376 MHz, Methanol-d₄) δ −77.53 (s, 3F), −118.19 (dd, J=8.8, 4.0 Hz, 1F). LCMS-ESI⁺ (m/z): [M+H]⁺ calculated for C₁₅H₂₃FN₅O: 308.19; found: 308.12; t_{R}=1.46 min on LC/MS Method A.

### Example 95

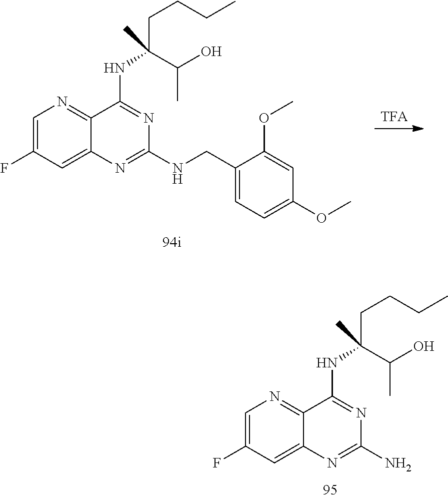 [SMILES: CCCC[C@@](C)(Nc1nc(NCc2ccc(OC)cc2OC)nc2cc(F)cnc12)C(C)O ; CCCC[C@@](C)(Nc1nc(N)nc2cc(F)cnc12)C(C)O]

Synthesis of (2R,3R)-3-((2-amino-7-fluoropyrido[3,2-d]pyrimidin-4-yl)amino)-3-methylheptan-2-ol (95). Compound 94l (10.3 mg, 23.4 umol) was dissolved in TFA (1 mL) and stirred at rt for 1 h. After the reaction mixture was carefully concentrated to dryness in vacuo, the residue was triturated with 50% aq. methanol and filtered through Celite-membrane filter. The filtrate was subjected to preparative HPLC (Gemini 10u C18110A, AXIA; 10% aq. acetonitrile-70% aq. acetonitrile with 0.1% TFA, over 20 min. gradient). The product fractions were combined, concentrated under reduced pressure, co-evaporated with methanol (10 mL×3), and dried under vacuum overnight to obtain compound 95 as its TFA salt. ¹H NMR (400 MHz, Methanol-d₄) δ 8.53 (d, J=2.4 Hz, 1H), 8.41 (s, 1H), 7.62 (dd, J=8.7, 2.5 Hz, 1H), 4.24 (q, J=6.4 Hz, 1H), 2.14 (ddd, J=15.0, 11.3, 4.2 Hz, 1H), 2.04 (dq, J=14.3, 5.2 Hz, 1H), 1.48 (s, 3H), 1.39-1.24 (m, 4H), 1.22 (d, J=6.4 Hz, 3H), 0.89 (t, J=7.0 Hz, 3H). ¹⁹F NMR (376 MHz, Methanol-d₄) δ −77.52 (s, 3F), −118.31 (dd, J=8.7, 4.1 Hz, 1F). LCMS-ESI⁺ (m/z): [M+H]⁺ calculated for C₁₅H₂₃FN₅O: 308.19; found: 308.12; t_{R}=1.47 min on LC/MS Method A.

### Example 96

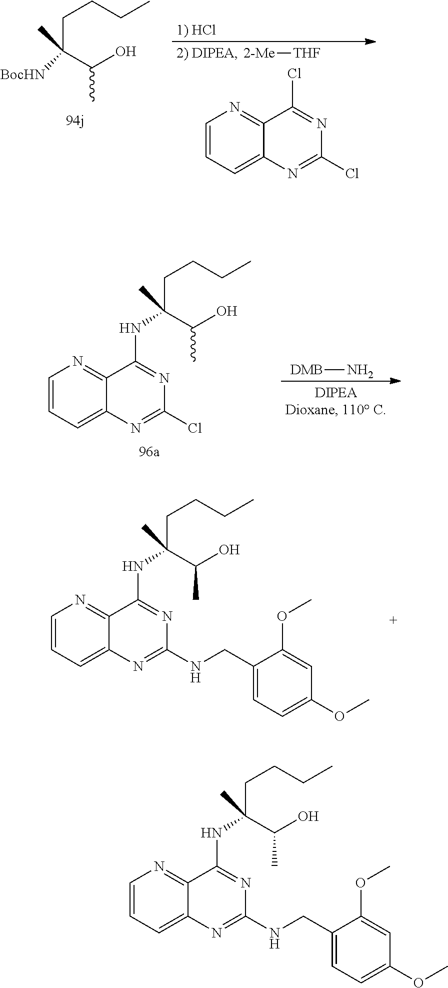

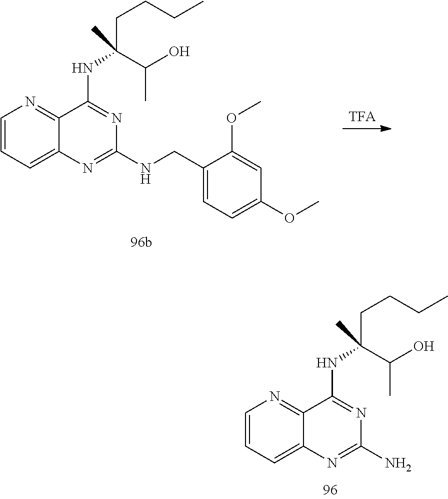 [SMILES: CCCC[C@@](C)(Nc1nc(NCc2ccc(OC)cc2OC)nc2cccnc12)C(C)O ; CCCC[C@@](C)(Nc1nc(N)nc2cccnc12)C(C)O]

Synthesis of (3R)-3-((2-chloropyrido[3,2-d]pyrimidin-4-yl)amino)-3-methylheptan-2-ol (96a). Compound 94j (195.7 mg, 0.798 mmol) was dissolved in 4M HCl in dioxane (3 mL) and stirred at rt for 1 h. The reaction mixture was then concentrated in vacuo. The residue was treated with 2-methyltetrahydrofuran (5 mL), 2,4-dichloropyrido[3,2-d]pyrimiidine (160 mg, 0.525 mmol) and N,N-diisopropylethylamine (0.57 mL, 3.272 mmol) and heated with an 80° C. bath for 3 h. The reaction mixture was cooled to rt, concentrated under reduced pressure and the residue was subjected to silica gel chromatography eluting with 0-100% EtOAc in hexanes to obtain compound 96a as a mixture of two diastereomers (˜2:3 ratio). LCMS-ESI⁺ (m/z): [M+H]⁺ calculated for C₁₅H₂₂ClN₄O: 309.15; found: 309.08; TR=1.41 mmn on LC/MS Method A.

Synthesis of (2S,3R)-3-((2-((2,4-dimethoxybenzyl)amino)pyrido[3,2-d]pyrimidin-4-yl)amino)-3-methylheptan-2-ol and (2R,3R)-3-((2-((2,4-dimethoxybenzyl)amino)pyrido[3,2-d]pyrimidin-4-yl)amino)-3-methylheptan-2-ol (96b and 96c). To a solution of compound 96a (132.6 mg, 0.429 mmol) in dioxane (5 mL) was added N,N-diisopropylethylamine (0.23 mL, 1.320 mmol) and 2,4-dimethoxybenzylamine (0.16 mL, 1.065 mmol), and the resulting mixture refluxed at 110° C. for 20 h. The reaction mixture was diluted with EtOAc (30 mL) and washed with water (30 mL×2). The aqueous fractions were back extracted with EtOAc (50 mL). The organic fractions were combined, dried over MgSO₄, filtered and then concentrated under reduced pressure. The residue was subjected to silica gel chromatography eluting with 0-100% EtOAc in hexanes to obtain a mixture of compounds 96b and 96c. The mixture was further subjected to chiral SFC (SFC IC-5 um-4.6×100 mm, 40% EtOH-ammonia, flow rate=3 mL/min) to obtain compound 96b, eluting first, and compound 96c, eluting second.

Compound 96b: ¹H NMR (400 MHz, Chloroform-d) δ 8.28 (dd, J=4.2, 1.5 Hz, 1H), 7.69 (d, J=8.4 Hz, 1H), 7.43 (dd, J=8.5, 4.3 Hz, 1H), 7.29 (d, J=8.2 Hz, 1H), 7.19 (s, 1H), 6.46 (d, J=2.4 Hz, 1H), 6.42 (dd, J=8.2, 2.4 Hz, 1H), 5.3 (br, 1H), 4.56 (d, J=5.7 Hz, 2H), 3.86 (m, 1H), 3.83 (s, 3H), 3.79 (s, 3H), 1.98 (m, 1H), 1.66-1.53 (m, 1H), 1.48 (s, 3H), 1.44-1.30 (m, 4H), 1.17 (d, J=6.4 Hz, 3H), 0.98-0.89 (m, 3H). LCMS-ESI⁺ (m/z): [M+H]⁺ calculated for C₂₄H₃₄N₅O₃: 440.27; found: 440.25; TR=0.99 min on LC/MS Method A.

Compound 96c: ¹H NMR (400 MHz, Chloroform-d) δ 8.29 (dd, J=4.2, 1.5 Hz, 1H), 7.70 (d, J=8.4 Hz, 1H), 7.43 (dd, J=8.5, 4.2 Hz, 1H), 7.30 (d, J=8.2 Hz, 1H), 7.16 (s, 1H), 6.46 (d, J=2.3 Hz, 1H), 6.42 (dd, J=8.2, 2.4 Hz, 1H), 5.25 (s, 1H), 4.56 (d, J=5.7 Hz, 2H), 3.84 (s, 3H), 3.79 (s, 3H), 3.86-3.75 (m, 1H), 2.13 (t, J=13.0 Hz, 1H), 1.93-1.79 (m, 1H), 1.52-1.40 (m, 1H), 1.38 (s, 3H), 1.35-1.15 (m, 3H), 1.20 (d, J=6.4 Hz, 3H), 0.87 (t, J=7.2 Hz, 3H). LCMS-ESI⁺ (m/z): [M+H]⁺ calculated for C₂₄H₃₄N₅O₃: 440.27; found: 440.25; t_{R}=1.00 min on LC/MS Method A.

Synthesis of (3R)-3-((2-aminopyrido[3,2-d]pyrimidin-4-yl)amino)-3-methylheptan-2-ol (96). Compound 96b (8.7 mg, 19.79 umol) was dissolved in TFA (1 mL) and stirred at rt for 1 h. The reaction mixture was concentrated under reduced pressure to dryness and then co-evaporated with methanol (10 mL). The resulting residue was dissolved in methanol (1 mL) and concentrated ammonium hydroxide (0.1 mL). The reaction mixture was stirred for 10 min. and then concentrated under reduced pressure to dryness and co-evaporated with methanol (10 mL). The residue was triturated with 50% aq. MeOH (10 mL) and filtered through a Celite-membrane filter. The filtrate was subjected to preparative HPLC (Gemini 10u C18110A, AXIA; 10% aq. acetonitrile-70% aq. acetonitrile with 0.1% TFA, over 20 min. gradient). The product fractions were combined, concentrated in vacuo, co-evaporated with methanol (10 mL×3), and dried under high-vacuum to provide compound 96 as its TFA salt. ¹H NMR (400 MHz, Methanol-d₄) δ 8.61 (dd, J=4.4, 1.5 Hz, 1H), 7.82 (dd, J=8.5, 1.5 Hz, 1H), 7.76 (dd, J=8.5, 4.4 Hz, 1H), 4.36 (q, J=6.5 Hz, 1H), 2.30 (dt, J=16.3, 6.8 Hz, 1H), 1.91-1.78 (m, 1H), 1.56 (s, 3H), 1.43-1.30 (m, 4H), 1.23 (d, J=6.5 Hz, 3H), 0.98-0.85 (m, 3H). LCMS-ESI⁺ (m/z): [M+H]⁺ calculated for C₁₅H₂₄N₅O: 290.20; found: 290.11; t_{R}=0.74 min on LC/MS Method A.

### Example 97

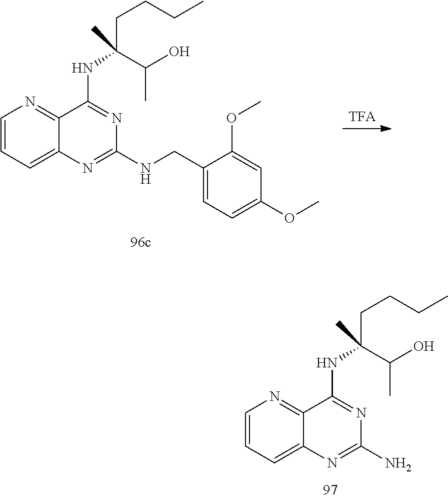 [SMILES: CCCC[C@@](C)(Nc1nc(NCc2ccc(OC)cc2OC)nc2cccnc12)C(C)O ; CCCC[C@@](C)(Nc1nc(N)nc2cccnc12)C(C)O]

Synthesis of (3R)-3-((2-aminopyrido[3,2-d]pyrimidin-4-yl)amino)-3-methylheptan-2-ol (97). Compound 96c (9.0 mg, 20.5 umol) was dissolved in TFA (1 mL) and stirred at rt for 1 h. The reaction mixture was carefully concentrated under reduced pressure to dryness and co-evaporated with methanol (10 mL). The residue was dissolved in methanol (1 mL) and concentrated ammonium hydroxide (0.1 mL). The reaction mixture was stirred for 10 min. and then concentrated under reduced pressure to dryness and then co-evaporated with methanol (10 mL). The resulting residue was triturated with 50% aq. methanol and filtered through a Celite-membrane filter. The filtrate was then subjected to preparative HPLC (Gemini 10u C18110A, AXIA; 10% aq. acetonitrile-70% aq. acetonitrile with 0.1% TFA, over 20 min. gradient). The product fractions were combined, concentrated under reduced pressure, co-evaporated with methanol (10 mL×3), and dried under high-vacuum to provide compound 97 as its TFA salt. ¹H NMR (400 MHz, Methanol-d₄) δ 8.61 (dd, J=4.3, 1.3 Hz, 1H), 7.82 (dd, J=8.5, 1.4 Hz, 1H), 7.76 (dd, J=8.5, 4.3 Hz, 1H), 4.26 (q, J=6.4 Hz, 1H), 2.11 (dddd, J=24.9, 19.8, 12.8, 7.0 Hz, 2H), 1.49 (s, 3H), 1.40-1.24 (m, 4H), 1.22 (d, J=6.4 Hz, 3H), 0.89 (t, J=6.9 Hz, 3H). LCMS-ESI⁺ (m/z): [M+H]⁺ calculated for C₁₅H₂₄N₅O: 290.20; found: 290.10; t_{R}=0.74 min on LC/MS Method A.

### Example 98

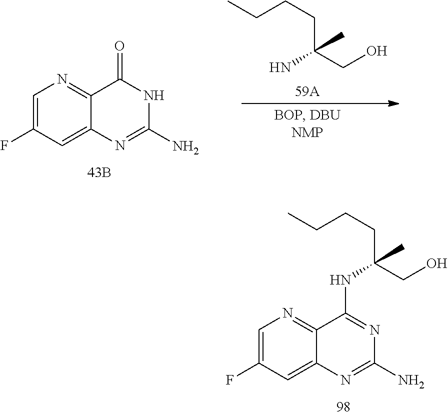

Synthesis of (R)-2-((2-amino-7-fluoropyrido[3,2-d]pyrimidin-4-yl)amino)-2-methylhexan-1-ol (98). Intermediate 43B (101 mg, 0.56 mmol) and (R)-α-Me-norleucinol 59A (109 mg, 0.83 mmol) were added to NMP (5.5 mL) followed by BOP reagent (0.36 g, 0.83 mmol) and DBU (0.25 mL, 1.67 mmol). The reaction mixture was stirred at rt for 16 h, and then diluted with EtOH (2 mL) and water (2 mL). The resulting mixture was subjected directly to HPLC purification (Gemini 10u C18110A, AXIA; 10% aq. acetonitrile-80% aq. acetonitrile with 0.1% TFA, over 20 min. gradient) to provide, after collection of product fractions and removal of solvent in vacuo, compound 98 as a TFA salt. ¹H NMR (400 MHz, Methanol-d4) δ 8.55 (d, J=2.4 Hz, 1H), 8.22 (s, 1H), 7.64 (dd, J=8.7, 2.5 Hz, 1H), 3.97 (d, J=11.2 Hz, 1H), 3.71 (d, J=11.2 Hz, 1H), 2.09 (m, 1H), 1.92 (m, 1H), 1.54 (s, 3H), 1.40-1.31 (m, 4H), 1.00-0.85 (m, 3H). ¹⁹F NMR (376 MHz, Methanol-d4) δ −77.68, −118.20 (d, J=8.8 Hz). LCMS-ESI⁺ (m/z): [M+H]⁺ calculated for C₁₄H₂₀FN₅O: 293.34; found: 294.1; t_{R}=0.68 min.

### Example 99

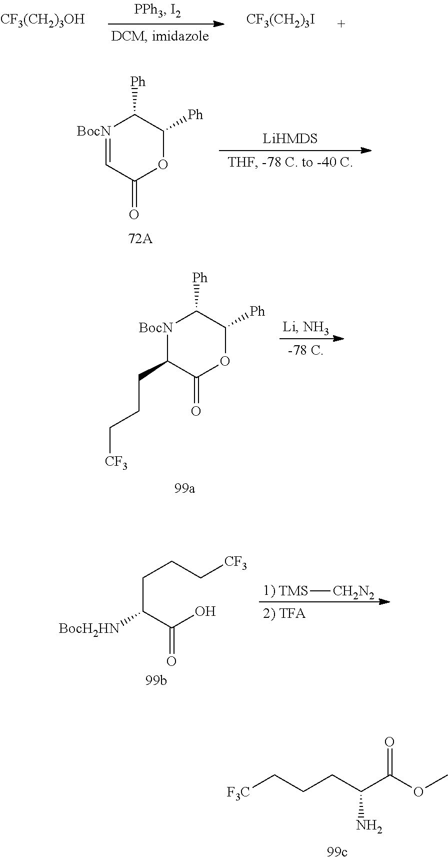

Synthesis of (3R,5R,6S)-tert-butyl 2-oxo-5,6-diphenyl-3-(4,4,4-trifluorobutyl)morpholine-4-carboxylate (99a). Imidazole (1.75 g, 0.03 mol), and triphenylphosphine, 99+% (6.08 g, 0.02 mol) were stirred in DCM (100 mL) under argon and cooled to 0° C. for 10 minutes. Iodine (5.94 g, 0.02 mol) was added over 5 minutes and the reaction was stirred at 0° C. for 20 minutes. A solution of 4,4,4-trifluoro-1-butanol, 97% (2.48 mL, 0.02 mol) was slowly added. The reaction was stirred and allowed to warm to rt. After 16 h, pentane (200 mL) was added and the resulting solids filtered off. Solvent was partially removed under reduced pressure, and then additional cold pentane (50 mL) was added. The solids were filtered off and the eluent concentrated under reduced pressure to afford 1,1,1-trifluoro-4-iodobutane.

(2S,3R)-tert-butyl 6-oxo-2,3-diphenylmorpholine-4-carboxylate, 72A (1 g, 2.83 mmol) and 1,1,1-trifluoro-4-iodobutane (2.02 g, 8.49 mmol) were dissolved in THF (24 mL) and HMPA (2.5 mL), and the mixture was then cooled to −78° C. under argon. 1M lithium hexamethyldisilazide (1.0M THF in THF, 4.24 mL) was added and the reaction transferred to a −40° C. bath. The cold bath was recharged with dry ice and the reaction left to warm to ambient temperature with stirring overnight. The reaction was quenched with EtOAc (25 mL) and poured into a mixture of EtOAc (100 mL) and saturated aqueous solution of NH₄Cl (50 mL). The organic layer was separated and washed with water (100 mL), brine (100 mL), dried over Na₂SO₄, filtered and concentrated under reduced pressure. The residue was subjected to silica gel chromatography eluting with hexanes-EtOAc to provide (3R,5R,6S)-tert-butyl 2-oxo-5,6-diphenyl-3-(4,4,4-trifluorobutyl)morpholine-4-carboxylate 99a.

Synthesis of (R)-2-((tert-butoxycarbonyl)amino)-6,6,6-trifluorohexanoic acid (99b). Lithium (granular), (157.24 mg, 22.65 mmol) was cooled in a −40° C. bath. Ammonia gas was slowly condensed via a cold finger into the reaction for 15-20 minutes. After an additional 20 minutes (3R,5R,6S)-tert-butyl 2-oxo-5,6-diphenyl-3-(4,4,4-trifluorobutyl)morpholine-4-carboxylate, 99a (700 mg, 1.51 mmol) in THF (10 mL) and EtOH (0.5 mL) was added. The reaction was allowed to warm to rt, and the liquid ammonia allowed to evaporate with stirring overnight. The resulting residue was treated with THF (50 mL) and water (50 mL) and stirred until all the solids dissolved. A saturated aq. ammonium chloride (50 mL) solution was added followed by 1N NaOH to adjust the pH to basic. The reaction mixture was washed with diethyl ether (100 mL), and the aqueous layer was then pH adjusted with 1N HCl to ˜pH 4. The aq. layer was then extracted with EtOAc (3×50 mL). The combined organics were then washed with ammonium chloride (50 mL), water (50 mL), brine (50 mL), dried over sodium sulfate, filtered and concentrated under reduced pressure to provide 99b.

Synthesis of (R)-methyl 2-amino-6,6,6-trifluorohexanoate (99c). Compound 99b (230 mg, 0.81 mmol) was dissolved in DCM (10 mL) and MeOH (1 mL). A solution of 2M (Trimethylsilyl) Diazomethane, 2M solution in hexanes (0.6 mL, 1.2 mmol) was added dropwise. The reaction was allowed to stir for 20 minutes and then 2 drops of acetic acid were added. The reaction mixture was concentrated under reduced pressure and the resulting residue treated with DCM (5 mL) and TFA (5 mL). The mixture was stirred for 90 minutes and then concentrated under reduced pressure. The residue was co-evaporated with DCM (20 mL×2) to provide 99c as its TFA salt.

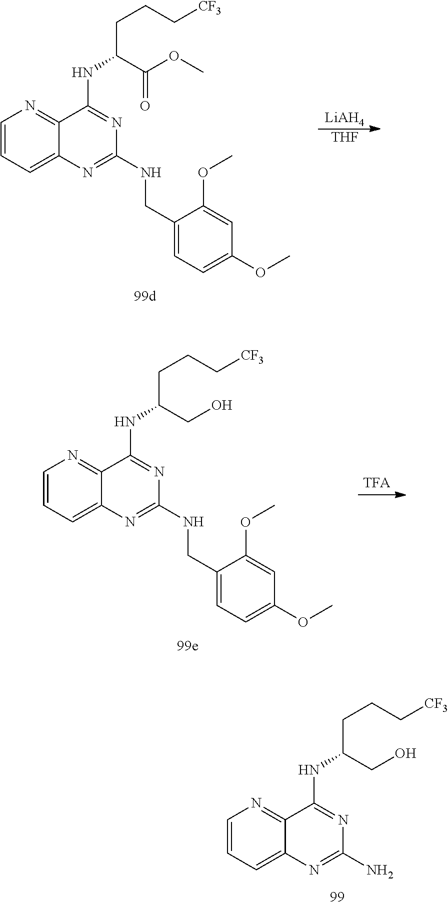 [SMILES: COC(=O)[C@@H](CCCC(F)(F)F)Nc1nc(NCc2ccc(OC)cc2OC)nc2cccnc12 ; "LiAH4" ; COc1ccc(CNc2nc(N[C@@H](CO)CCCC(F)(F)F)c3ncccc3n2)c(OC)c1 ; "THF" ; Nc1nc(N[C@@H](CO)CCCC(F)(F)F)c2ncccc2n1]

Synthesis of (R)-methyl 2-((2-((2,4-dimethoxybenzyl)amino)pyrido[3,2-d]pyrimidin-4-yl)amino)-6,6,6-trifluorohexanoate (99d). 99d was synthesized in a similar fashion to compound 63B, instead replacing 63A with (R)-methyl 2-amino-6,6,6-trifluorohexanoate TFA salt 99c (100 mg, 0.75 mmol), to obtain 99d. MS (m/z) 494.2 [M+H]⁺; t_{R}=0.95 min.

Synthesis of (R)-2-((2-((2,4-dimethoxybenzyl)amino)pyrido[3,2-d]pyrimidin-4-yl)amino)-6,6,6-trifluorohexan-1-ol (99e). Compound 99d (100 mg, 0.2 mmol) was treated with THF (15 mL) and cooled to 0° C. under argon. To this solution was added 1M LiAlH₄ in THF (0.61 mL, 0.61 mmol) and the reaction mixture stirred at 0° C. Upon completion, the reaction was diluted in EtOAc/H₂O and extracted with EtOAc (50 mL×3). The combined organics were then washed with aq. ammonium chloride (50 mL), water (50 mL), brine (50 mL), dried over Na₂SO₄, filtered and concentrated under reduced pressure. The crude residue was subjected to silica gel chromatography eluting with hexanes-EtOAc to afford 99e. LCMS (m/z) 466.1 [M+H]⁺. t_{R}=1.14 min.

Synthesis of (R)-2-((2-aminopyrido[3,2-d]pyrimidin-4-yl)amino)-6,6,6-trifluorohexan-1-ol (99). Compound 99e (75 mg, 0.16 mmol) was dissolved in TFA (5 mL) and allowed to stir for 1 h. The TFA was removed under reduced pressure and MeOH (10 mL) was added. The mixture was stirred for 1 h and then filtered. The eluent was removed in vacuo and the residue was treated with MeOH (10 mL). The mixture was stirred for 16 h and then concentrated under reduced pressure. The residue was co-evaporated with MeOH (10 mL, ×3) and the resulting residue dried under high vacuum to afford compound 99 as its TFA salt. ¹H NMR (400 MHz, Methanol-d₄) δ 8.65 (dd, J=4.4, 1.4 Hz, 1H), 7.84 (dd, J=8.5, 1.4 Hz, 1H), 7.77 (dd, J=8.5, 4.4 Hz, 1H), 4.56 (ddt, J=10.9, 5.5, 3.1 Hz, 1H), 3.75 (d, J=5.3 Hz, 2H), 2.40-2.07 (m, 2H), 1.94-1.76 (m, 2H), 1.66 (dddd, J=19.0, 16.1, 8.7, 5.9 Hz, 2H). ¹⁹F NMR (376 MHz, Methanol-d₄) δ −68.49 (t, J=11.0 Hz), −77.91. LCMS-ESI⁺ (m/z): [M+H]⁺ calculated for C₁₃H₁₆F₃N₅O: 315.29; found: 316.2; t_{R}=0.82 min.

### Example 100

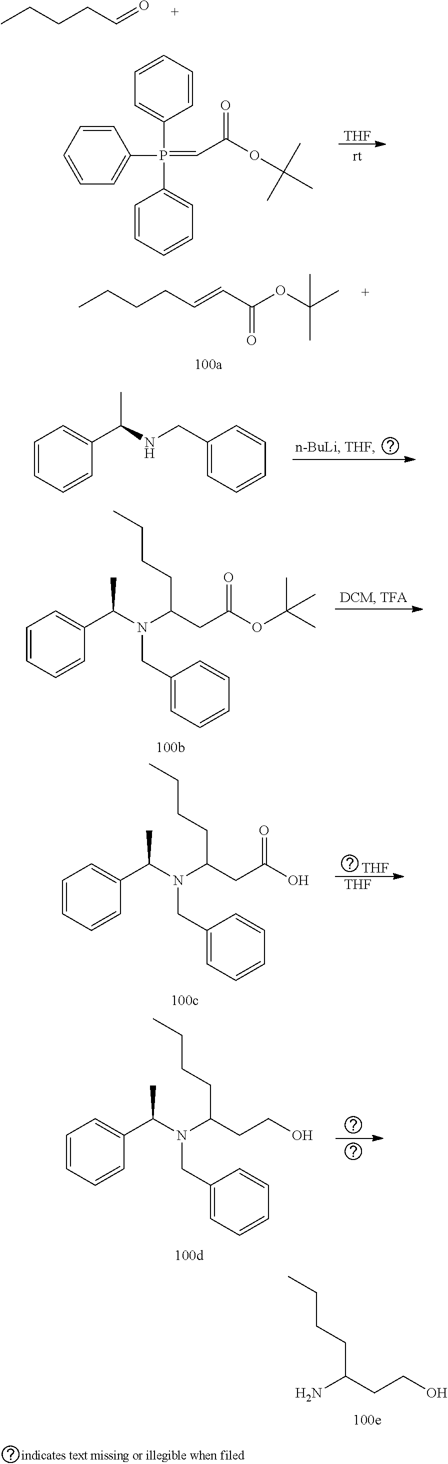

Synthesis of (E)-tert-butyl hept-2-enoate (100a). To a solution of valeraldehyde (2.82 mL, 26.57 mmol) in THF (50 mL) was added (tert-butoxycarbonylmethylene)triphenylphosphorane (10 g, 26.57 mmol) and the reaction mixture stirred for 16 h at rt. The solvents were then removed under reduced pressure, and the residue slurried in diethyl ether and filtered. The filtrate was concentrated in vacuo and the residue subjected to silica gel chromatography eluting with hexanes-EtOAc to give 100a. ¹H NMR (400 MHz, Methanol-d4) δ 6.85 (dt, J=15.5, 7.0 Hz, 1H), 5.73 (dt, J=15.6, 1.6 Hz, 1H), 2.26-2.11 (m, 2H), 1.52-1.25 (m, 13H), 0.93 (t, J=7.2 Hz, 3H).

Synthesis of (R)-tert-butyl 3-(benzyl((S)-1-phenylethyl)amino)heptanoate (100b). 2.5M Butyllithium (2.5M in Hexanes, 14.33 mL) was added to a stirred solution of (R)-(+)-N-benzyl-alpha-methylbenzylamine (7.99 mL, 38.2 mmol) in THF (100 mL) at −78° C. The reaction mixture was stirred for 30 minutes, and then 100a (4.4 g, 23.88 mmol) in THF (50 mL) was added slowly to the reaction mixture. The reaction mixture was then stirred at −78° C. for 2 h, quenched with sat. aq. NH₄Cl solution (100 mL) and allowed to warm to rt. EtOAc (200 mL) and water (100 mL) were added, and the organic layer separated. The aqueous layer was extracted with EtOAc (3×50 mL) and the combined organics were washed with brine (100 mL), dried over Na₂SO₄, filtered and concentrated in vacuo. The resulting residue was subjected to silica gel chromatography eluting with hexanes-EtOAc to provide 100b. ¹H NMR (400 MHz, Methanol-d4) δ 7.41 (d, J=7.2 Hz, 2H), 7.36-7.10 (m, 8H), 3.87-3.73 (m, 2H), 3.50 (d, J=15.0 Hz, 1H), 3.24 (tt, J=9.4, 4.2 Hz, 1H), 2.04 (dd, J=14.4, 3.6 Hz, 1H), 1.89 (dd, J=14.4, 9.4 Hz, 1H), 1.57-1.43 (m, 3H), 1.38 (s, 8H), 1.33-1.12 (m, 7H), 0.87 (t, J=7.3 Hz, 3H).

Synthesis of (R)-3-(benzyl((S)-1-phenylethyl)amino)heptanoic acid (100c). (R)-tert-butyl 3-(benzyl((S)-1-phenylethyl)amino)heptanoate 100b (6.4 g, 16.18 mmol) was dissolved in DCM (40 mL) and treated with TFA (20 mL). The reaction mixture was allowed to stir at 40° C. for 24 h and then concentrated under reduced pressure to provide 100c. LCMS (m/z) 340.0 [M+H]⁺. t_{R}=0.94 min

Synthesis of (R)-3-(benzyl((S)-1-phenylethyl)amino)heptan-1-ol (100d). (R)-3-(benzyl((S)-1-phenylethyl)amino)heptanoic acid 100c (5.5 g, 16.2 mmol) was dissolved in THF (100 mL) under argon, and 1M borane-tetrahydrofuran in THF (64.81 mL, 64.81 mmol) was slowly added. The reaction was allowed to stir for several h at rt. MeOH was slowly added to quench the reaction and the mixture was allowed to stir for an additional 20 minutes. A ˜2N HCl (aq) (14 mL) solution was added and the mixture concentrated under reduced pressure to afford a white solid. The solid material was suspended in DCM (100 mL) and filtered. The filter cake was rinsed with DCM (25 mL). The mother liquor was concentrated under reduced pressure to afford a light yellow oil which was subjected to silica gel chromatography eluting with DCM-MeOH to afford 100d. MS (m/z) 326.1 [M+H]⁺; t_{R}=0.82 min

Synthesis of (R)-3-aminoheptan-1-ol (100e). (R)-3-(benzyl((S)-1-phenylethyl)amino)heptan-1-ol 100d (0.78 g, 2.4 mmol) was treated with EtOH (25 mL) and 20% Pd(OH)₂/C (300 mg, 0.43 mmol). The reaction vessel was purged 3× with H₂ gas and then allowed to stir for 2 days under H₂. The reaction mixture was filtered and solvents were removed under reduced pressure to afford 100e. ¹H NMR (400 MHz, Methanol-d4) δ 3.90-3.68 (m, 2H), 3.39-3.27 (m, 1H), 1.98-1.72 (m, 2H), 1.72-1.57 (m, 3H), 1.39 (h, J=4.5, 4.0 Hz, 4H), 1.03-0.86 (m, 3H).

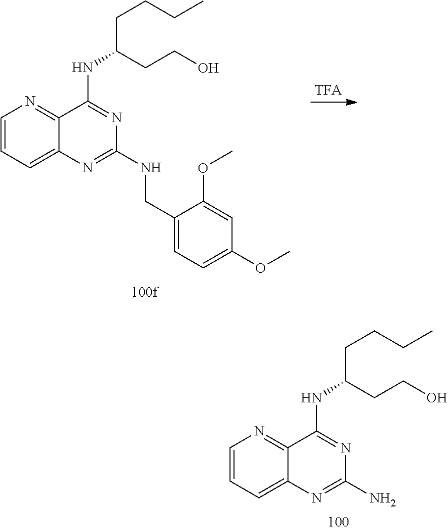 [SMILES: CCCC[C@H](CCO)Nc1nc(NCc2ccc(OC)cc2OC)nc2cccnc12 ; CCCC[C@H](CCO)Nc1nc(N)nc2cccnc12]

Synthesis of (R)-3-((2-aminopyrido[3,2-d]pyrimidin-4-yl)amino)heptan-1-ol (100). 2,4-dichloropyrido[3,2-d]pyrimidine (100 mg, 0.5 mmol) was reacted with 100e (65.6 mg, 0.5 mmol) followed by 2,4-dimethoxybenzylamine (150.21 μl, 1 mmol) as described for the synthesis of 59B from 59A, to prepare 100f. Compound 100f was then subjected to TFA (3 mL) for 1 h as described in the preparation of compound 59 from 59B to afford, 100 as its TFA salt. MS (m/z) 276.1 [M+H]⁺; t_{R}=0.64 min; ¹H NMR (400 MHz, Methanol-d4) δ 8.63 (dd, J=4.4, 1.5 Hz, 1H), 7.82 (dd, J=8.5, 1.5 Hz, 1H), 7.76 (dd, J=8.5, 4.4 Hz, 1H), 4.64 (tt, J=7.9, 5.6 Hz, 1H), 3.72-3.59 (m, 2H), 1.99-1.83 (m, 2H), 1.81-1.66 (m, 2H), 1.46-1.29 (m, 4H), 0.97-0.82 (m, 3H). ¹⁹F NMR (376 MHz, Methanol-d4) δ −77.56.

### Example 101

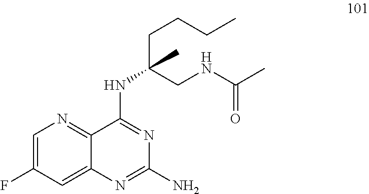

Synthesis of (R)—N-(2-((2-amino-7-fluoropyrido[3,2-d]pyrimidin-4-yl)amino)-2-methylhexyl)acetamide (101). Compound 101 was prepared following the procedure described in Example 84, using 2,4-dichloro-7-fluoropyrido[3,2-d]pyrimidine 84E (30 mg, 0.14 mmol) and reacting sequentially with (R)—N-(2-amino-2-methylhexyl)acetamide hydrochloride 61E (28.72 mg, 0.14 mmol) followed by 2,4-dimethoxybenzylamine (82.69 μl, 0.55 mmol). The resulting product was then subjected to TFA treatment as described in the preparation of 84 from 84G, to provide 101 as its TFA salt. MS (m/z) 335.2 [M+H]⁺; t_{R}=0.64 min; ¹H NMR (400 MHz, Methanol-d4) δ 8.54 (t, J=2.9 Hz, 2H), 7.62 (dd, J=8.8, 2.5 Hz, 1H), 3.99-3.86 (m, 1H), 3.51 (d, J=14.0 Hz, 1H), 2.26-2.05 (m, 1H), 1.95 (s, 4H), 1.54 (s, 3H), 1.45-1.27 (m, 4H), 0.99-0.80 (m, 3H); ¹⁹F NMR (376 MHz, Methanol-d4) δ −78.04, −118.27 (d, J=8.8 Hz).

### Example 102

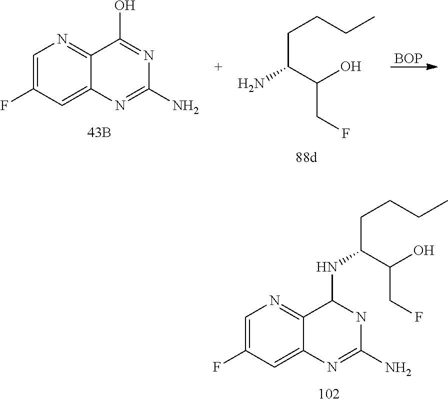

Synthesis of (3R)-3-((2-amino-7-fluoropyrido[3,2-d]pyrimidin-4-yl)amino)-1-fluoroheptan-2-ol (102). A solution of compound 43B (131.5 mg, 0.730 mmol), compound 88d (212.2 mg, 1.415 mmol), and BOP (392.7 mg, 0.888 mmol) in DMF (7 mL) was stirred at rt as DBU (0.33 mL, 2.209 mmol) was added. The reaction mixture was stirred at rt for 17.5 h, diluted with water (7 mL), and then the mixture was filtered. The filtrate was subjected to preparative HPLC (Gemini 10u C18110A, AXIA; 10% aq. acetonitrile-70% aq. acetonitrile with 0.1% TFA, over 20 min. gradient) and the product fractions were combined, concentrated under reduced pressure to obtain the crude product. The crude product was re-subjected to preparative HPLC (Gemini 10u C18 110A, AXIA; 10% aq. acetonitrile-70% aq. acetonitrile with 0.1% TFA, over 20 min. gradient), and the combined product fractions concentrated under reduced pressure, co-evaporated with methanol (10 mL×4), and dried to obtain compound 102 as its TFA salt. ¹H NMR (400 MHz, Methanol-d₄) δ 8.67 (d, J=9.6 Hz, 0H), 8.55 (d, J=2.4 Hz, 1H), 7.65 (dd, J=8.8, 2.5 Hz, 1H), 4.63-4.54 (m, 1H), 4.51-4.39 (m, 1H), 4.39-4.26 (m, 1H), 4.03 (dddd, J=16.5, 6.0, 4.9, 3.2 Hz, 1H), 1.87-1.73 (m, 2H), 1.49-1.28 (m, 4H), 0.98-0.83 (m, 3H). ¹⁹F NMR (376 MHz, Methanol-d₄) δ −77.71, −117.85 (d, J=8.3 Hz), −231.37 (td, J=47.3, 16.5 Hz). LCMS-ESI⁺ (m/z): [M+H]⁺ calculated for C₁₄H₂₀F₂N₅O: 312.16; found: 312.16; t_{R}=0.70 min.

### Example 103

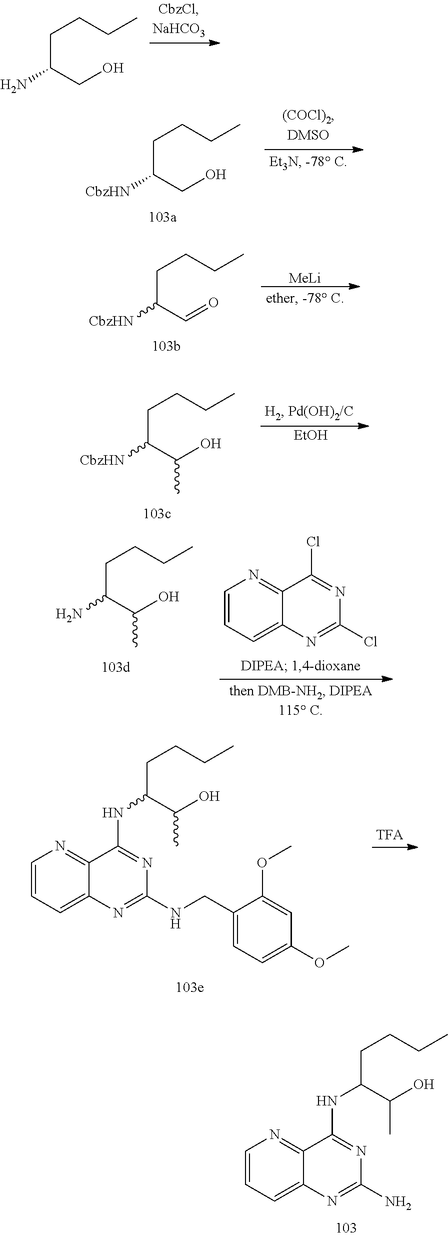

Synthesis of (R)-benzyl (1-hydroxyhexan-2-yl)carbamate (103a). A solution of (R)-2-aminohexan-1-ol (1.853 g, 15.81 mmol) and sodium bicarbonate (1961.6 mg, 31.63 mmol) in water (80 mL) was stirred at rt and benzyl chloroformate (2.7 mL, 95% purity, 18.98 mmol) was added. After stirring for 1 h at rt, the mixture was extracted with EtOAc (100 mL×1, 80 mL×2). The combined extracts were washed with brine, dried (Na₂SO₄), filtered and concentrated in vacuo. The residue was subjected to silica gel chromatography eluting with 0-100% EtOAc in hexanes to obtain 103a. ¹H NMR (400 MHz, Methanol-d₄) δ 7.44-7.18 (m, 5H), 6.75 (d, J=8.7 Hz, 0H), 5.07 (d, J=2.2 Hz, 2H), 3.57 (dt, J=11.1, 5.4 Hz, 1H), 3.48 (d, J=5.6 Hz, 2H), 1.58 (dq, J=14.0, 8.4, 6.4 Hz, 1H), 1.35 (dq, J=14.3, 7.4, 6.4 Hz, 5H), 0.91 (t, J=5.6 Hz, 3H). LCMS-ESI⁺ (m/z): [M+H]⁺ calculated for C₁₄H₂₂NO₃: 252.16; found: 251.80; t_{R}=0.90 min.

Synthesis of benzyl (1-oxohexan-2-yl)carbamate (103b). To a stirred solution of oxalyl chloride (0.125 mL, 1.432 mmol) in DCM (10 mL) cooled with an −78° C. bath was added DMSO (0.203 mL, 2.865 mmol) in DCM (2 mL) over 8 min. After 15 min, a solution of compound 103a (300 mg, 1.194 mmol) in DCM (4 mL) was added to the reaction mixture. The mixture was stirred at −78° C. for 30 min. and then triethylamine (0.832 mL, 5.968 mmol) was added with vigorous stirring. The resulting mixture was allowed to warm to rt, diluted with DCM (20 mL), washed with water (30 mL×3), brine (20 mL), dried (MgSO₄), filtered and concentrated under reduced pressure. The residue was subjected to silica gel chromatography eluting with 0-50% EtOAc in hexanes to obtain 103b. ¹H NMR (400 MHz, Methanol-d₄) δ 9.41 (d, J=80.7 Hz, 0H), 7.51-7.06 (m, 5H), 5.08 (d, J=2.1 Hz, 2H), 4.43 (d, J=3.9 Hz, 1H), 3.57 (dd, J=9.8, 5.1 Hz, 1H), 1.65 (dd, J=11.3, 6.7 Hz, 1H), 1.46-1.20 (m, 5H), 0.90 (t, J=6.3 Hz, 3H). LCMS-ESI⁺ (m/z): [M+H]⁺ calculated for C₁₄H₂₀NO₃: 250.14; found: 249.83; t_{R}=0.93 min.

Synthesis of benzyl (2-hydroxyheptan-3-yl)carbamate (103c). To a solution of compound 103b (277.0 mg, 1.111 mmol) dissolved in diethyl ether (10 mL) and cooled to −78° C. was added dropwise 1.57 M methyllithium in diethyl ether (1.557 mL, 2.444 mmol). After 10 min, saturated ammonium chloride (10 mL) was added to the reaction mixture and the resulting mixture was allowed to warm to rt for 45 min. The mixture was extracted with EtOAc (50 mL×3), the combined organic extracts were washed with brine, dried over MgSO₄, filtered and concentrated in vacuo. The residue was subjected to silica gel chromatography eluting with 0-70% EtOAc in hexanes to obtain compound 103c as a mixture of 4 diastereomers. ¹H NMR (400 MHz, Methanol-d₄) δ 7.44-7.19 (m, 5H), 5.08 (d, J=3.0 Hz, 2H), 3.83-3.57 (m, 1H), 3.54-3.40 (m, 1H), 1.76-1.41 (m, 2H), 1.43-1.24 (m, 6H), 1.12 (dd, J=9.4, 6.4 Hz, 3H), 0.90 (dd, J=7.9, 4.9 Hz, 3H). LCMS-ESI⁺ (m/z): [M+H]⁺ calculated for C₁₅H₂₄NO₃: 266.18; found: 265.81; t_{R}=0.93 min.

Synthesis of 3-aminoheptan-2-ol (103d). Compound 103c (59.6 mg, 0.225 mmol) and 20% Pd(OH)₂ on carbon (15.2 mg) were dissolved in EtOH (2 mL) and stirred under H₂ atmosphere. After 2 h, the reaction mixture was filtered through Celite pad and the removed solid was washed with EtOH (10 mL). The filtrate and washing were concentrated under reduced pressure and the crude compound, 103d, was used without further purification. LCMS-ESI⁺ (m/z): [M+H]⁺ calculated for C₇H₁₈NO: 132.14; found: 131.91; t_{R}=0.37 min.

Synthesis 3-((2-((2,4-dimethoxybenzyl)amino)pyrido[3,2-d]pyrimidin-4-yl)amino)heptan-2-ol (103e). To a solution of compound 103d (29.5 mg, 0.225 mmol) and 2,4-dichloropyrido[3,2-d]pyrimidine (37.4 mg, 0.187 mmol) in dioxane (2 mL) was added N,N-diisopropylethylamine (0.05 mL, 0.281 mmol). After 20 min, additional N,N-diisopropylethylamine (0.080 mL, 0.449 mmol) and 2,4-dimethoxybenzylamine (0.10 mL, 0.674 mmol) were added and the resulting mixture was heated at 115° C. bath for 7 h. The reaction mixture was allowed to cool to rt, diluted with water (50 mL), extracted with DCM (25 mL×2). The combined organic extracts were washed with water (25 mL×2), dried over MgSO₄, filtered and then concentrated in vacuo. The residue was subjected to silica gel chromatography eluting with 0-100% EtOAc in hexanes to obtain compound 103e. ¹H NMR (400 MHz, Methanol-d₄) δ 8.31 (dt, J=4.3, 1.0 Hz, 0.85H), 8.05 (s, 0.15H), 7.63 (s, 1H), 7.48 (dd, J=8.5, 4.2 Hz, 1H), 7.18 (dd, J=8.3, 1.9 Hz, 1H), 6.52 (d, J=2.3 Hz, 1H), 6.48-6.38 (m, 1H), 4.64-4.47 (m, 2H), 4.35-4.21 (m, 1H), 4.00-3.87 (m, 1H), 3.83 (two s, 3H), 3.76 (two s, 3H), 3.35 (s, 1H), 1.90-1.52 (m, 2H), 1.33 (m, 4H), 1.16 (m, 3H), 0.97-0.78 (m, 3H). LCMS-ESI⁺ (m/z): [M+H]⁺ calculated for C₂₃H₃₄N₅O₃: 426.25; found: 426.17; t_{R}=1.00 min.

Synthesis of 3-((2-aminopyrido[3,2-d]pyrimidin-4-yl)amino)heptan-2-ol (103). Compound 103e (17.4 mg, 40.9 umol) was dissolved in TFA (1 mL) and stirred at rt for 1 h. The reaction mixture was concentrated under reduced pressure and co-evaporated with MeOH (10 mL). The resulting residue was dissolved in MeOH (1 mL) and concentrated ammonium hydroxide (0.1 mL). The mixture was stirred for 10 min. at rt and then concentrated under reduced pressure to dryness. The residue was dissolved in DMF-water (1:1, 5 mL) and filtered through a Celite/membrane filter. The filtrate was subjected to preparative HPLC (Gemini 10u C18110A, AXIA; 10% aq. acetonitrile-70% aq. acetonitrile with 0.1% TFA, over 20 min. gradient). The product fractions were combined, concentrated under reduced pressure, co-evaporated with methanol (10 mL×3), and dried under high vacuum to obtain compound 103 as its TFA salt. ¹H NMR (400 MHz, Methanol-d₄) δ 8.64 (dt, J=4.4, 1.2 Hz, 1H), 7.84 (dt, J=8.5, 1.4 Hz, 1H), 7.77 (ddd, J=8.5, 4.4, 1.5 Hz, 1H), 4.47-4.31 (m, 1H), 3.99 (tq, J=6.5, 3.5 Hz, 0.5H), 3.94 (dd, J=6.6, 5.5 Hz, 0.5H), 1.95-1.82 (m, 0.5H), 1.82-1.72 (m, 1H), 1.72-1.63 (m, 0.5H), 1.48-1.25 (m, 4H), 1.22 (d, J=6.4 Hz, 1.5H), 1.19 (d, J=6.4 Hz, 1.5H), 0.89 (two d, J=6.9, Hz each, 3H). LCMS-ESI⁺ (m/z): [M+H]⁺ calculated for C₁₄H₂₂N₅O: 276.18; found: 276.15; t_{R}=0.68 min.

### Example 104

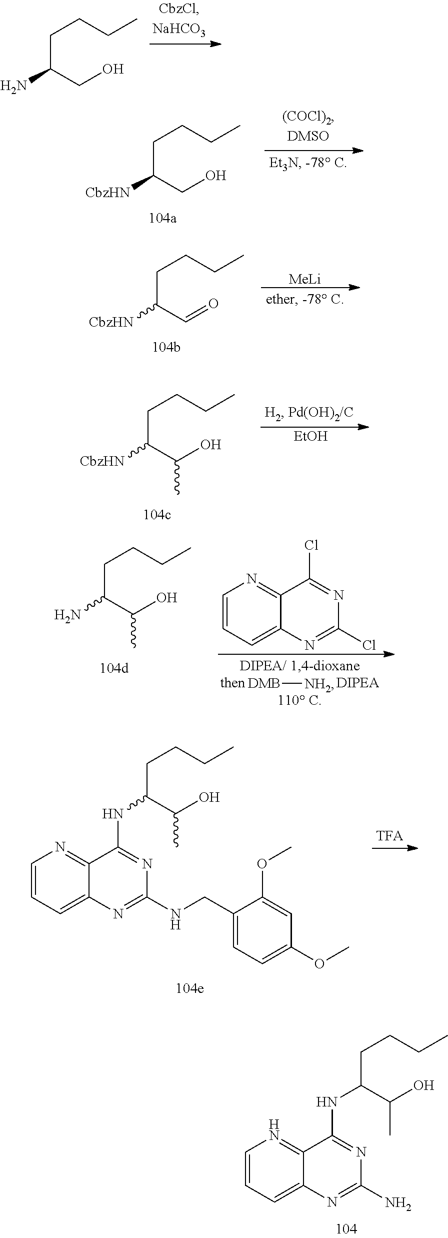

Synthesis of (S)-benzyl (1-hydroxyhexan-2-yl)carbamate (104a). To a mixture of (S)-2-aminohexan-1-ol (504.4 mg, 4.30 mmol) and sodium bicarbonate (533.9 mg, 8.61 mmol) in water (20 mL) was added benzyl chloroformate (0.74 mL, 95% purity, 5.17 mmol). The resulting mixture was vigorously stirred at rt overnight. The solid was dissolved with EtOAc (75 mL) and the mixture extracted with EtOAc (75 mL×2). The organic extracts were combined, dried over Na₂SO₄, filtered and concentrated in vacuo to obtain white solids. The solids were subjected to silica gel chromatography eluting with 0-100% EtOAc in hexanes to obtain compound 104a. ¹H NMR (400 MHz, Methanol-d₄) δ 7.42-7.22 (m, 5H), 5.07 (d, J=2.1 Hz, 2H), 3.59 (d, J=8.0 Hz, 1H), 3.48 (d, J=5.6 Hz, 2H), 1.59 (d, J=10.8 Hz, 1H), 1.34 (td, J=15.4, 11.8, 7.3 Hz, 6H), 0.91 (t, J=6.0 Hz, 3H). LCMS-ESI⁺ (m/z): [M+H]⁺ calculated for C₁₄H₂₂NO₃: 252.16; found: 251.78; t_{R}=0.88 min.

Synthesis of benzyl (1-oxohexan-2-yl)carbamate (104b). To a stirred solution of oxalyl chloride (0.052 mL, 0.602 mmol) in DCM (1.5 mL) at −78° C. was added DMSO (0.086 mL, 1.205 mmol) in DCM (2 mL) over 8 min. After 15 min, a solution of compound 104a (108.1 mg, 0.430 mmol) in DCM (1.5 mL) was added to the reaction mixture. The mixture was stirred at −78° C. for 30 min. and then triethylamine (0.174 mL, 1.248 mmol) was added with vigorous stirring. The resulting mixture was allowed to warm to rt over 45 min. The mixture was diluted with DCM (30 mL), washed with water (30 mL×3), brine (25 mL), dried over MgSO₄, filtered and concentrated under reduced pressure to obtain the mixture 104b. LCMS-ESI⁺ (m/z): [M+H]⁺ calculated for C₁₄H₂₀NO₃: 250.14; found: 249.79; t_{R}=0.91 min.

Synthesis of benzyl (2-hydroxyheptan-3-yl)carbamate (104c). To a solution of compound 104b (107.3 mg, 0.430 mmol), dissolved in diethyl ether (4 mL) and cooled to −78° C. was added 1.57 M methyllithium in diethyl ether (0.685 mL, 1.076 mmol) dropwise. After 10 min, saturated aq. ammonium chloride (7 mL) was added to the reaction mixture and the resulting mixture was allowed to warm to rt for 45 min. The mixture was extracted with EtOAc (25 mL×2), and the combined organic extracts washed with brine, dried over MgSO₄, filtered and concentrated in vacuo. The residue was subjected to silica gel chromatography eluting with 0-70% EtOAc in hexanes to obtain compound 104c as a mixture of 4 diastereomers. ¹H NMR (400 MHz, Methanol-d₄) δ 7.42-7.20 (m, 5H), 6.63 (dd, J=102.5, 9.6 Hz, 1H), 5.08 (d, J=3.3 Hz, 2H), 3.80-3.54 (m, 1H), 3.52-3.41 (m, 1H), 1.75-1.42 (m, 2H), 1.42-1.27 (m, 5H), 1.12 (dd, J=9.3, 6.4 Hz, 3H), 0.90 (d, J=3.5 Hz, 3H). LCMS-ESI⁺ (m/z): [M+H]⁺ calculated for C₁₅H₂₄NO₃: 266.18; found: 265.81; t_{R}=1.06 min.

Synthesis of 3-aminoheptan-2-ol (104d). Compound 104c (71.68 mg, 0.270 mmol) and 20% Pd(OH)₂ on carbon (19 mg) were dissolved in EtOH (2 mL) and stirred under H₂ atmosphere. After 2 h, the reaction mixture was filtered through Celite pad and the removed solid washed with EtOH (5 mL). The filtrate and washings were concentrated under reduced pressure to provide 104d that was used without further purification. LCMS-ESI⁺ (m/z): [M+H]⁺ calculated for C₇H₁₈NO: 132.14; found: 131.91; t_{R}=0.51 min.

Synthesis of 3-((2-((2,4-dimethoxybenzyl)amino)pyrido[3,2-d]pyrimidin-4-yl)amino)heptan-2-ol (104e). To a solution of compound 104d (35.45 mg, 0.270 mmol) and 2,4-dichloropyrido[3,2-d]pyrimidine (5.02 mg, 0.225 mmol) in dioxane (3 mL) was added N,N-diisopropylethylamine (0.06 mL, 0.338 mmol). After 20 min. additional N,N-diisopropylethylamine (0.096 mL, 0.540 mmol) and 2,4-dimethoxybenzylamine (0.120 mL, 0.811 mmol) were added and the resulting mixture was heated at 115° C. bath for 6 h. The reaction mixture was cooled to rt, diluted with water (30 mL), and extracted with DCM (20 mL×2). The organic extracts were combined, washed with water (30 mL×2), brine (25 mL), dried over MgSO₄, filtered and concentrated under reduced pressure. The residue was subjected to silica gel chromatography eluting with 0-100% EtOAc in hexanes to obtain compound 104e. ¹H NMR (400 MHz, Methanol-d₄) δ 8.31 (ddd, J=4.2, 1.5, 0.8 Hz, 1H), 7.63 (d, J=8.4 Hz, 1H), 7.48 (dd, J=8.5, 4.2 Hz, 1H), 7.25-7.08 (m, 1H), 6.60-6.37 (m, 2H), 4.84 (s, 3H), 4.54 (d, J=5.3 Hz, 2H), 4.35-4.22 (m, 1H), 3.83 (d, J=10.3 Hz, 3H), 3.79-3.73 (m, 3H), 1.88-1.52 (m, 2H), 1.46-1.28 (m, 4H), 1.23-1.12 (m, 3H), 0.86 (td, J=7.0, 2.2 Hz, 3H). LCMS-ESI⁺ (m/z): [M+H]⁺ calculated for C₂₃H₃₄N₅O₃: 426.25; found: 426.19; t_{R}=0.97 min.

Synthesis of 3-((2-aminopyrido[3,2-d]pyrimidin-4-yl)amino)heptan-2-ol (104). Compound 104e (27.3 mg, 64.2 umol) was dissolved in TFA (1 mL) and stirred at rt for 1 h. The reaction mixture was concentrated under reduced pressure and co-evaporated with MeOH (10 mL). The resulting residue was dissolved in MeOH (1 mL) and concentrated ammonium hydroxide (0.1 mL). The reaction mixture was stirred at rt, and then concentrated under reduced pressure to dryness. The residue was treated with DMF-water (1:1, 5 mL). The insoluble material was removed via filtration through a Celite/membrane filter, and the filtrate was subjected to preparative HPLC (Gemini 10u C18110A, AXIA; 10% aq. acetonitrile-70% aq. acetonitrile with 0.1% TFA, over 20 min. gradient). The fractions were combined, concentrated under reduced pressure, co-evaporated with methanol (10 mL×3), and dried in vacuum overnight to obtain 104 as its TFA salt. ¹H NMR (400 MHz, Methanol-d₄) δ 8.64 (dt, J=4.4, 1.2 Hz, 1H), 7.84 (dt, J=8.5, 1.4 Hz, 1H), 7.77 (ddd, J=8.5, 4.4, 1.5 Hz, 1H), 4.46-4.40 (m, 0.5H), 4.37 (m, 1H), 4.00 (m, 0.5H), 3.97-3.88 (m, 0.5H), 1.88 (m, 0.5H), 1.82-1.72 (m, 1H), 1.72-1.62 (m, 0.5H), 1.48-1.25 (m, 4H), 1.22 (d, J=6.4 Hz, 1.5H), 1.19 (d, J=6.4 Hz, 1.5H), 0.89 (two t, J=6.8 Hz each, 3H). LCMS-ESI⁺ (m/z): [M+H]⁺ calculated for C₁₄H₂₂N₅O: 276.18; found: 276.15; t_{R}=0.68 min.

### Example 105

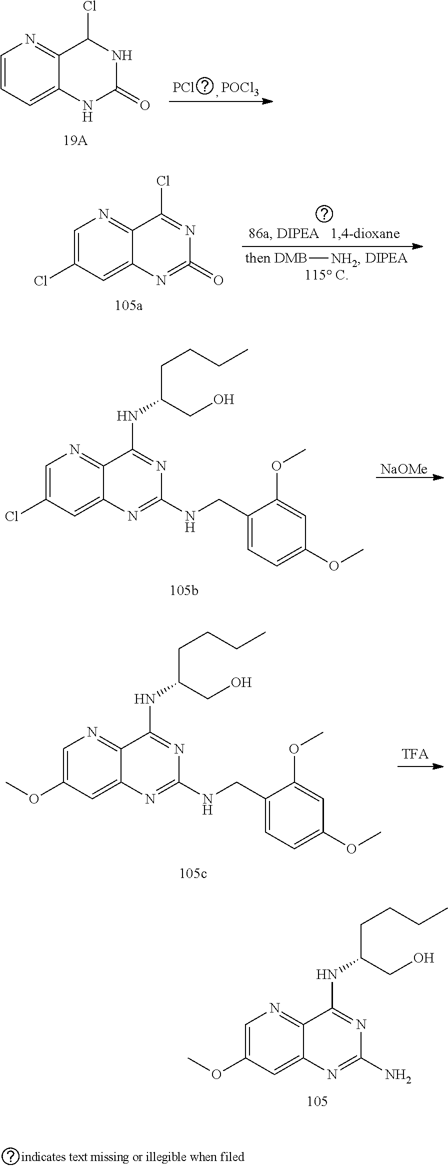

Synthesis of 2,4,7-trichloropyrido[3,2-d]pyrimidine (105a). A mixture of pyrido[3,2-d]pyrimidine-2,4(1H,3H)-dione 19A (supplied by Astatech, Inc., 2.00 g, 12.26 mmol), phosphorus pentachloride (15.32 g, 73.56 mmol) and phosphorus oxychloride (22.86 mL, 245.20 mmol) in a sealed, thick-walled reaction tube, was stirred at 160° C. for 5 h. The mixture was concentrated in vacuo and the residue was dissolved in DCM (100 mL). The organic solution was washed with water (100 mL), brine (100 mL), dried over MgSO₄, filtered and then concentrated in vacuo. The residue was subjected to silica gel chromatography eluting with 0-50% EtOAc in hexanes to obtain compound 105a. ¹H NMR (400 MHz, Chloroform-d) δ 9.02 (d, J=2.2 Hz, 21H), 8.29 (d, J=2.2 Hz, 21H). LCMS-ESI⁺ (m/z): t_{R}=0.86 min.

Synthesis of (R)-2-((7-chloro-2-((2,4-dimethoxybenzyl)amino)pyrido[3,2-d]pyrimidin-4-yl)amino)hexan-1-ol (105b). To a solution of compound 105a (336 mg, 1.066 mmol) and (R)-2-aminohexan-1-ol 86a (137.5 mg, 1.173 mmol) in dioxane (4 ML) was added N,N-diisopropylethylamine (0.23 mL, 1.292 mmol). The mixture was stirred for 40 min. and then additional N,N-diisopropylethylamine (0.38 mL, 2.132 mmol) and 2,4-dimethoxybenzylamine (0.473 mL, 3.198 mmol) were added. The resulting mixture was heated at 115° C. for 2 h. The reaction mixture was cooled to rt, diluted with water (30 mL) and extracted with DCM (30 mL). The organic extracts were washed with water (30 mL), brine (30 mL), dried over MgSO₄, filtered and concentrated in vacuo. The residue was subjected to silica gel chromatography eluting with 0-100% EtOAc in hexanes to obtain compound 105b. LCMS-ESI⁺ (m/z): [M+H]⁺ calculated for C₂₂H₂₉ClN₅O₃: 446.20; found: 446.23, t_{R}=0.80 min.

Synthesis of (R)-2-((2-((2,4-dimethoxybenzyl)amino)-7-methoxypyrido[3,2-d]pyrimidin-4-yl)amino)hexan-1-ol (105c). To a solution of compound 105b (50 mg, 0.113 mmol) in dioxane (2 mL) was added sodium methoxide (25 wt. %, 0.064 mL, 0.280 mmol) in a microwave vial. The resulting mixture was heated at 120° C. for 45 min. in a microwave reactor. The reaction mixture was concentrated in vacuo and the residue was dissolved in methanol (2 mL) and sodium methoxide (25 wt. %, 0.2 mL, 0.874 mmol). The resulting mixture was heated at 150° C. for 1 h in a microwave reactor. The reaction mixture was diluted with water (25 mL) and extracted with EtOAc (25 mL×2). The combined extracts were washed with saturated aqueous ammonium chloride (25 mL), dried over MgSO₄, filtered and concentrated under reduced pressure to obtain crude compound 105c. LCMS-ESI⁺ (m/z): [M+H]⁺ calculated for C₂₃H₃₂N₅O₄: 442.25; found: 442.23; t_{R}=0.82 min.

Synthesis of (R)-2-((2-amino-7-methoxypyrido[3,2-d]pyrimidin-4-yl)amino)hexan-1-ol (105). The compound 105c was dissolved in TFA (1 mL) and stirred at rt for 1 h. The reaction mixture was concentrated under reduced pressure and co-evaporated with MeOH (10 mL). The resulting residue was subjected to preparative HPLC (Gemini 10u C18110A, AXIA; 5% aq. acetonitrile-50% aq. acetonitrile with 0.1% TFA, over 20 min. gradient). The product fractions were concentrated in vacuo, co-evaporated with methanol (10 mL×3), and dried under vacuum to obtain compound 105 as its TFA salt. ¹H NMR (400 MHz, Methanol-d₄) δ 8.32 (d, J=2.5 Hz, 1H), 7.22 (d, J=2.6 Hz, 1H), 4.58-4.39 (m, 1H), 4.00 (s, 4H), 3.77-3.60 (m, 3H), 1.72 (dtd, J=14.7, 8.5, 8.0, 5.4 Hz, 2H), 1.51-1.22 (m, 5H), 1.00-0.80 (m, 4H). ¹⁹F NMR (376 MHz, Methanol-d₄) δ −77.51. LCMS-ESI⁺ (m/z): [M+H]⁺ calculated for C₁₄H₂₂N₅O₂: 292.18; found: 292.19; t_{R}=0.45 min.

### Example 106

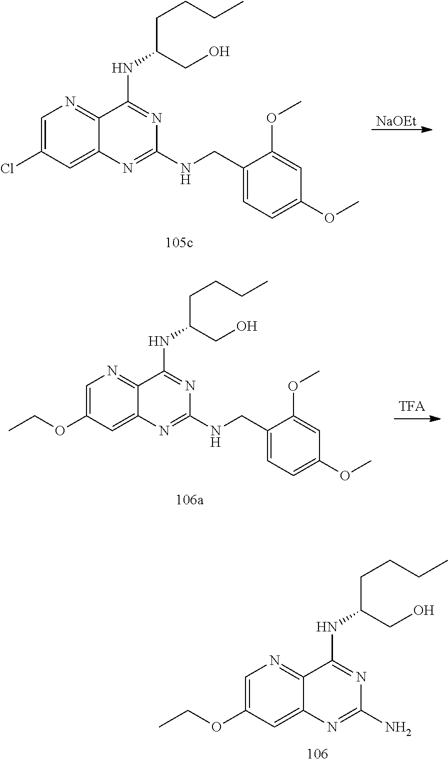

Synthesis of (R)-2-((2-((2,4-dimethoxybenzyl)amino)-7-ethoxypyrido[3,2-d]pyrimidin-4-yl)amino)hexan-1-ol (106a). To a solution of compound 105c (40 mg, 0.090 mmol) in EtOH (3 mL) was added sodium ethoxide (21 wt. %, 0.335 mL, 0.897 mmol) in a microwave vial. The resulting mixture was heated at 120° C. for 45 min. in a microwave reactor. The reaction mixture was concentrated in vacuo and the residue was then dissolved in water (25 mL) and EtOAc (25 mL). The organic layer was separated and washed with saturated aqueous ammonium chloride, dried over MgSO₄, filtered and then concentrated in vacuo to obtain crude compound 106a. LCMS-ESI⁺ (m/z): [M+H]⁺ calculated for C₂₄H₃₄N₅O₄: 456.26; found: 456.23; t_{R}=0.76 min.

Synthesis of (R)-2-((2-amino-7-ethoxypyrido[3,2-d]pyrimidin-4-yl)amino)hexan-1-ol (106). The compound 106a was dissolved in TFA (1 mL) and stirred at rt for 1 h. The reaction mixture was concentrated in vacuo and co-evaporated with MeOH (10 mL). The resulting residue was dissolved in MeOH (1 mL) and concentrated ammonium hydroxide (0.1 mL). The mixture was stirred at 50° C. for 10 min. and then concentrated under reduced pressure. The resulting residue was subjected to preparative HPLC (Gemini 10u C18110A, AXIA; 5% aq. acetonitrile-50% aq. acetonitrile with 0.1% TFA, over 20 min. gradient). The product fractions were concentrated in vacuo, co-evaporated with methanol (10 mL×3), and then dried under high vacuum to obtain compound 106 as its TFA salt. ¹H NMR (400 MHz, Methanol-d₄) δ 7.94 (d, J=2.6 Hz, 1H), 6.83 (d, J=2.6 Hz, 1H), 4.02 (q, J=7.0 Hz, 3H), 3.55 (d, J=4.9 Hz, 3H), 1.33 (t, J=7.0 Hz, 4H), 1.30-1.15 (m, 4H), 0.91-0.63 (m, 3H). ¹⁹F NMR (377 MHz, Methanol-d₄) δ −77.50. LCMS-ESI⁺ (m/z): [M+H]⁺ calculated for C₁₅H₂₄N₅O₂: 306.19; found: 306.20; t_{R}=0.51 min.

### Example 107

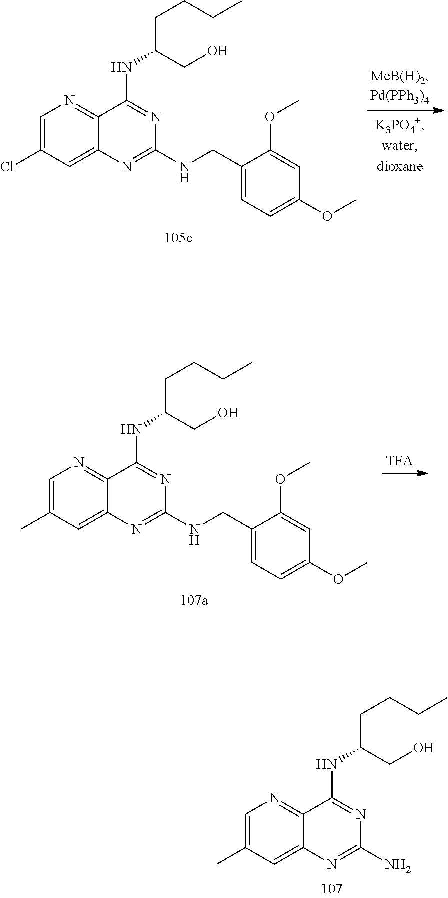

Synthesis of (R)-2-((2-((2,4-dimethoxybenzyl)amino)-7-methylpyrido[3,2-d]pyrimidin-4-yl)amino)hexan-1-ol (107a). A mixture of compound 105c (35 mg, 0.078 mmol), methylboronic acid (18.8 mg, 0.314 mmol), potassium phosphate tribasic (50.0 mg, 0.235 mmol), and palladium tetrakis(triphenylphosphine (18.14 mg, 0.016 mmol) in water (2 mL) and dioxane (2 mL) was stirred at 150° C. for 45 min. in a microwave reactor. The reaction mixture was diluted with water (25 mL) and extracted with EtOAc (25 mL). The organic layer was washed with water (25 mL), brine (25 mL), dried over MgSO₄, filtered and then concentrated under reduced pressure to obtain crude compound 107a. LCMS-ESI⁺ (m/z): [M+H]⁺ calculated for C₂₃H₃₂N₅O₃: 292.18; found: 426.22; t_{R}=0.70 min.

Synthesis of (R)-2-((2-amino-7-methylpyrido[3,2-d]pyrimidin-4-yl)amino)hexan-1-ol (107). The compound 107a was dissolved in TFA (1 mL) and stirred at rt for 1 h. The reaction mixture was concentrated in vacuo and the residue co-evaporated with MeOH (10 mL). The resulting residue was dissolved in MeOH (1 mL) and concentrated ammonium hydroxide (0.1 mL). The mixture was stirred for 10 min. at 50° C. and then concentrated under reduced pressure. The resulting residue was subjected to preparative HPLC (Gemini 10u C18110A, AXIA; 5% aq. acetonitrile-50% aq. acetonitrile with 0.1% TFA, over 20 min. gradient). The product fractions were concentrated in vacuo, co-evaporated with methanol (10 mL×3), and dried under high-vacuum to obtain compound 107 as its TFA salt. ¹H NMR (400 MHz, Methanol-d₄) δ 8.53-8.46 (m, 1H), 7.62 (tt, J=1.9, 1.0 Hz, 1H), 4.51 (dtd, J=9.0, 5.5, 3.1 Hz, 1H), 3.72 (d, J=5.3 Hz, 2H), 2.51 (d, J=2.2 Hz, 3H), 1.83-1.62 (m, 2H), 1.49-1.29 (m, 4H), 0.98-0.86 (m, 3H). ¹⁹F NMR (376 MHz, Methanol-d₄) δ −77.52. LCMS-ESI⁺ (m/z): [M+H]⁺ calculated for C₁₄H₂₂N₅O: 276.18; found: 276.16; t_{R}=0.50 min.

### Example 108

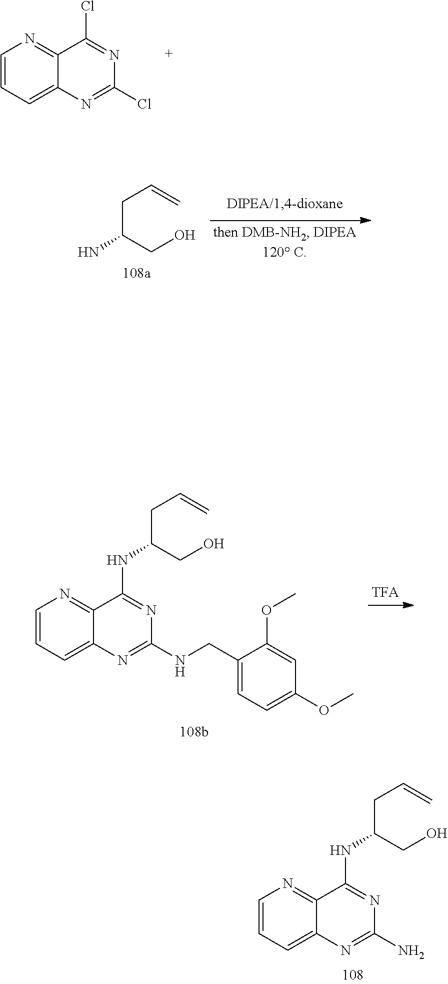

Synthesis of (R)-2-((2-((2,4-dimethoxybenzyl)amino)pyrido[3,2-d]pyrimidin-4-yl)amino)pent-4-en-1-ol (108b). To a solution of 2,4-dichloropyrido[3,2-d]pyrimidine (50 mg, 0.250 mmol) and (R)-2-aminopent-4-en-1-ol hydrochloride 108a (26.6 mg, 0.280 mmol, Chiralix B.V., Netherland) in dioxane (2 mL) was added N,N-diisopropylethylamine (0.09 mL, 0.500 mmol). The mixture was stirred overnight and then additional N,N-diisopropylethylamine (0.09 mL, 0.500 mmol) and 2,4-dimethoxybenzylamine (0.403 mL, 2.727 mmol) were added. The resulting mixture was heated at 120° C. overnight. The reaction mixture was allowed to cool to rt, diluted with water (25 mL) and extracted with EtOAc (25 mL×3). The organic extracts were washed with water (25 mL), brine (25 mL), dried over MgSO), filtered and then concentrated in vacuo to obtain the crude compound 108b. LCMS-ESI⁺ (m/z): [M+H]⁺ calculated for C₂₁H₂₆N₅O₃: 396.20; found: 396.14, t_{R}=0.69 min.

Synthesis of (R)-2-((2-aminopyrido[3,2-d]pyrimidin-4-yl)amino)pent-4-en-1-ol (108). The compound 108b (99 mg) was dissolved in TFA (3 mL) and stirred at rt for 3 h. The reaction mixture was concentrated under reduced pressure and co-evaporated with MeOH (10 mL). The resulting residue was subjected to preparative HPLC (Gemini 10u C18110A, AXIA; 5% aq. acetonitrile-50% aq. acetonitrile with 0.1% TFA, over 20 min. gradient). The product fractions were concentrated in vacuo, co-evaporated with methanol (10 mL×3), and dried under high vacuum to obtain compound 108 as its TFA salt. ¹H NMR (400 MHz, Methanol-d₄) δ 8.64 (dd, J=4.3, 1.5 Hz, 1H), 7.89-7.65 (m, 2H), 6.02-5.70 (m, 1H), 5.24-5.10 (m, 1H), 5.11-4.99 (m, 1H), 4.63-4.45 (m, 1H), 3.76 (d, J=5.3 Hz, 2H), 2.68-2.35 (m, 2H). ¹⁹F NMR (376 MHz, Methanol-d₄) δ −77.49. LCMS-ESI⁺ (m/z): [M+H]⁺ calculated for C₁₂H₁₆N₅O: 246.14; found: 246.09, t_{R}=0.45 min.

### Example 110

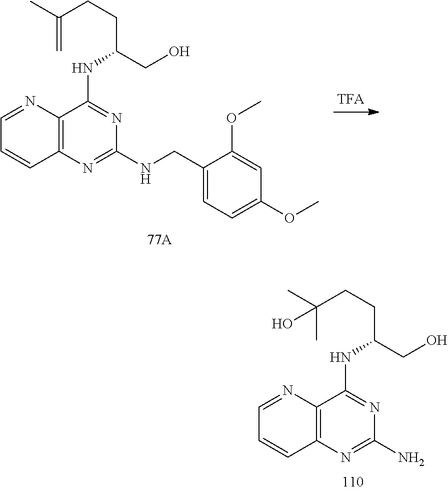 [SMILES: C=C(C)CC[C@H](CO)Nc1nc(NCc2ccc(OC)cc2OC)nc2cccnc12 ; CC(C)(O)CC[C@H](CO)Nc1nc(N)nc2cccnc12]

Synthesis of (R)-2-((2-amino-7-fluoropyrido[3,2-d]pyrimidin-4-yl)amino)-2-methylheptan-1-ol (110). To 77A (40 mg, 0.09 mmol) was added TFA (3 mL) and the mixture stirred for 2 h. The reaction mixture was concentrated under reduced pressure and the residue subjected to preparative HPLC (Synergi 4 u Polar-RP 80 A, Axia; 10% aq. acetonitrile-70% aq. acetonitrile with 0.1% TFA, over 20 min. gradient) to afford 110 as its TFA salt. LCMS (m/z): 292.12 [M+H]⁺; t_{R}=0.50 min. on LC/MS Method A. ¹H NMR (400 MHz, Methanol-d₄) δ 8.63 (dd, J=4.4, 1.4 Hz, 1H), 7.87 (dd, J=8.5, 1.4 Hz, 1H), 7.76 (dd, J=8.5, 4.4 Hz, 1H), 4.61-4.34 (m, 1H), 3.76 (d, J=5.3 Hz, 2H), 1.96-1.70 (m, 2H), 1.64-1.51 (m, 2H), 1.19 (s, 6H). ¹⁹F NMR (377 MHz, Methanol-d₄) δ −77.52.

### Example 111

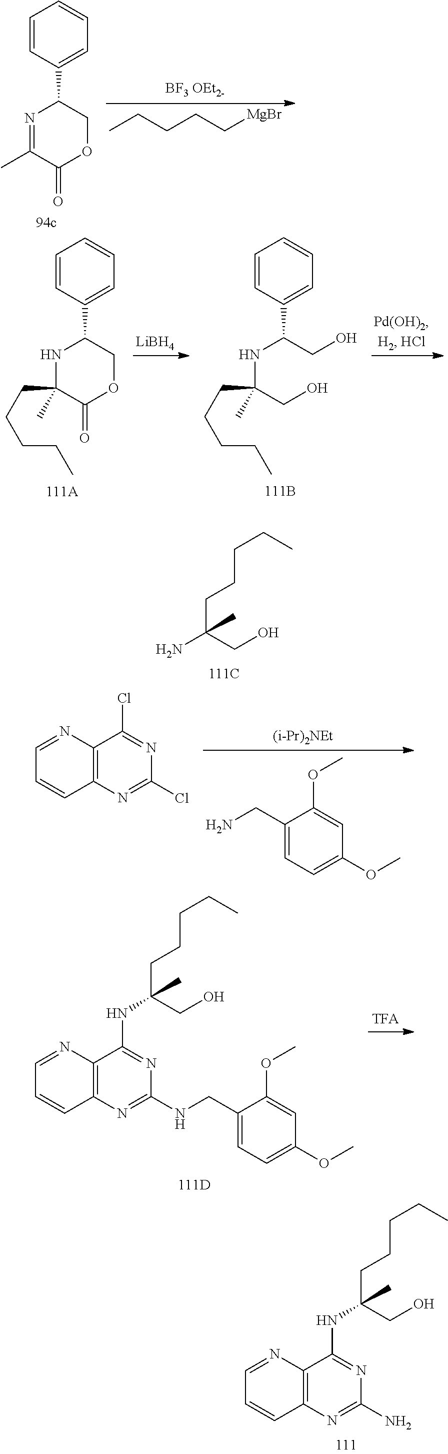

Synthesis of (3R,5R)-3-methyl-3-pentyl-5-phenylmorpholin-2-one (111A). To a solution of 94c (2 g, 10.57 mmol) in THF (50 ml) at −78° C. was added 2M boron trifluoride diethyl etherate in THF (2.76 ml, 22.39 mmol, 2.1 equiv.) over 10 minutes. After 90 minutes, 2M pentylmagnesium chloride solution in THF (11.19 ml, 22.38 mmol, 2.1 equiv.) was added slowly. The reaction was stirred for 2 h and then quenched with sat. NH₄Cl (200 mL). The mixture was allowed to warm to rt and then diluted with water (200 mL). The mixture was extracted with EtOAc (3×300 mL) and the combined extracts washed with water (3×500 mL), brine (300 mL), dried over NaSO₄, and concentrated under reduced pressure. The residue was subjected to silica gel chromatography eluting with hexanes-EtOAc to afford 111A. LCMS (m/z): 262.06 [M+H]⁺; t_{R}=1.14 min. on LC/MS Method A.

Synthesis of (R)-2-(((R)-2-hydroxy-1-phenylethyl)amino)-2-methylheptan-1-ol (111B). To a solution of 111A (1.65 g, 6.31 mmol) in THF (100 ml) at 0° C. was added 2M lithium borohydride in THF (6.35 ml, 12.7 mmol, 2 equiv.). The reaction was warmed to rt and stirred overnight. The mixture was then quenched with water (100 mL) and extracted with EtOAc (3×300 mL). The combined organics were washed with water (500 mL), brine (100 mL), dried over Na₂SO₄, and concentrated under reduced pressure to afford 111B that was used without further purification. LCMS (m/z): 266.05 [M+H]⁺; t_{R}=0.64 min. on LC/MS Method A.

Synthesis of (R)-2-amino-2-methylheptan-1-ol (111C). To a solution of 111B (1.66 g, 6.25 mmol) in EtOH (20 mL) was added Pd(OH)₂/C (20% wt %, 0.92 g) and 4M HCl in dioxane (2.37 ml, 9.50 mmol, 1.5 equiv.). The mixture was stirred under and atmosphere of H₂ at 70° C. overnight. The reaction was then filtered through Celite and concentrated to afford 111C that was used without further purification. LCMS (m/z): 145.95 [M+H]⁺; t_{R}=0.57 min. on LC/MS Method A.

Synthesis of (R)-2-((2-((2,4-dimethoxybenzyl)amino)pyrido[3,2-d]pyrimidin-4-yl)amino)-2-methylheptan-1-ol (111D). To 2,4-dichloropyrido[3,2-d]pyrimidine (118.89 mg, 0.59 mmol) in dioxane (12 mL) was added 111C (135 mg, 0.74 mmol, 1.25 equiv.), and N,N-diisopropylethylamine (0.78 ml, 4.46 mmol, 7.5 equiv.). The reaction mixture was stirred at 80° C. overnight. 2,4-dimethoxybenzylamine (0.27 ml, 1.85 mmol, 3.1 equiv.) was added and the mixture was heated to 100° C. for 6 h. The reaction mixture was allowed to cool, diluted with EtOAc (50 mL), washed with water (50 mL), saturated NH₄Cl (50 mL), dried over MgSO₄, filtered, and concentrated under reduced pressure. The residue was subjected to silica gel chromatography eluting with hexanes-EtOAc to afford 111D. LCMS (m/z): 440.30 [M+H]⁺; t_{R}=0.93 min. on LC/MS Method A.

Synthesis of (R)-2-((2-amino-7-fluoropyrido[3,2-d]pyrimidin-4-yl)amino)-2-methylheptan-1-ol (111). To 111D (155 mg, 0.35 mmol) was added TFA (3 mL). After 1 h, the reaction was concentrated under reduced pressure and the residue subjected to preparative HPLC (Synergi 4 u Polar-RP 80 A, Axia; 10% aq. acetonitrile-70% aq. acetonitrile with 0.1% TFA, over 20 min. gradient) to afford 111 as its TFA salt. LCMS (m/z): 290.15 [M+H]⁺; t_{R}=0.72 min. on LC/MS Method A. ¹H NMR (400 MHz, Methanol-d₄) δ 8.63 (dd, J=4.3, 1.5 Hz, 1H), 7.86-7.80 (m, 1H), 7.77 (dd, J=8.5, 4.3 Hz, 1H), 3.98 (d, J=11.2 Hz, 1H), 3.72 (d, J=11.2 Hz, 1H), 2.16-2.04 (m, 1H), 1.92 (tt, J=11.1, 4.9 Hz, 1H), 1.55 (s, 3H), 1.42-1.28 (m, 7H), 0.93-0.85 (m, 3H). ¹⁹F NMR (377 MHz, Methanol-d₄) δ −77.58.

### Example 112

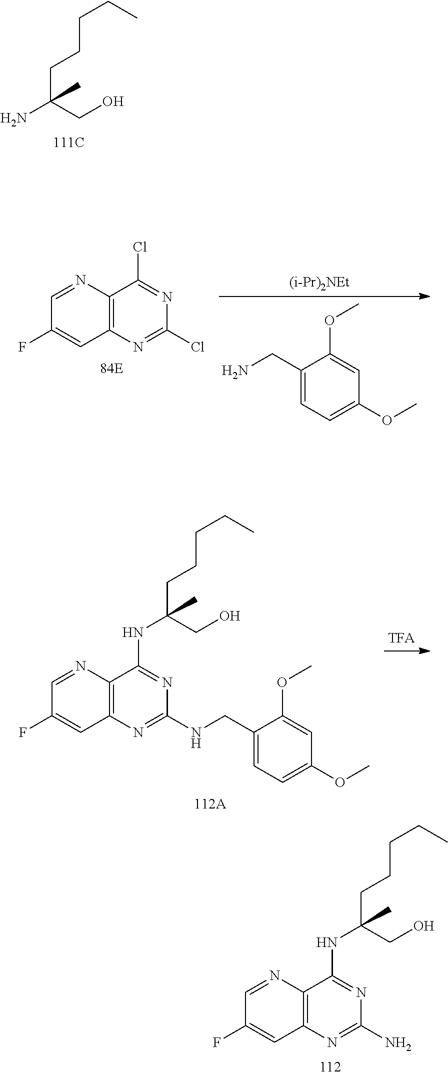

Synthesis of (R)-2-((2-((2,4-dimethoxybenzyl)amino)-7-fluoropyrido[3,2-d]pyrimidin-4-yl)amino)-2-methylheptan-1-ol (112A). To a solution of 84E (119.98 mg, 0.55 mmol) in dioxane (10 mL) was added 111C (125 mg, 0.69 mmol, 1.25 equiv.) and N,N-diisopropylethylamine (0.72 ml, 4.13 mmol, 6 equiv.). The mixture was stirred at 80° C. overnight. 2,4-dimethoxybenzylamine (0.2 ml, 1.38 mol, 2.5 equiv.) was added and the reaction heated to 100° C. for 6 h. The reaction mixture was allowed to cool, diluted with EtOAc (50 mL), washed with water (50 mL), sat. NH₄Cl (50 mL), dried over MgSO₄, filtered, and concentrated under reduced pressure. The residue was subjected to silica gel chromatography eluting with hexanes-EtOAc to afford 112A. LCMS (m/z): 458.26 [M+H]⁺; t_{R}=1.00 min. on LC/MS Method A.

Synthesis of (R)-2-((2-amino-7-fluoropyrido[3,2-d]pyrimidin-4-yl)amino)-2-methylheptan-1-ol (112). To 112A (105 mg, 0.23 mmol) was added TFA (3 mL). After 1 h, the reaction mixture was concentrated under reduced pressure and subjected to preparative HPLC (Synergi 4 u Polar-RP 80 A, Axia; 10% aq. acetonitrile-70% aq. acetonitrile with 0.1% TFA, over 20 min. gradient) to afford 112 as its TFA salt. LCMS (m/z): 308.14 [M+H]⁺; t_{R}=0.75 min. on LC/MS Method A. ¹H NMR (400 MHz, Methanol-d₄) δ 8.54 (d, J=2.5 Hz, 1H), 8.22 (s, 1H), 7.62 (ddd, J=8.7, 2.4, 0.8 Hz, 1H), 3.96 (d, J=11.2 Hz, 1H), 3.70 (d, J=11.2 Hz, 1H), 2.13-2.02 (m, 1H), 1.91 (s, 1H), 1.53 (s, 3H), 1.41-1.28 (m, 7H), 0.93-0.84 (m, 3H). ¹⁹F NMR (377 MHz, Methanol-d₄) δ −77.56, −118.19 (dd, J=8.7, 4.2 Hz).

### Example 113

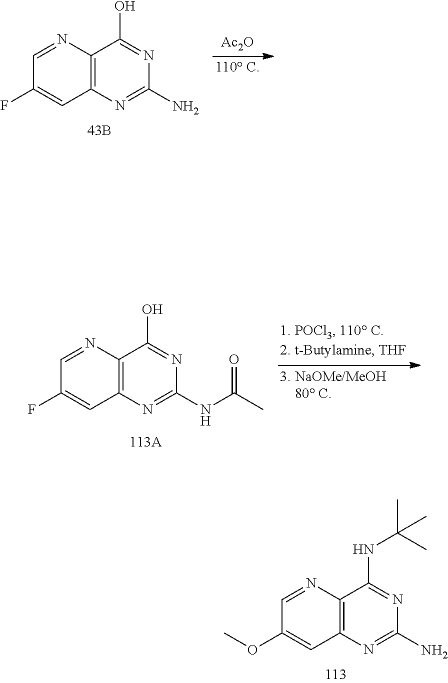

Synthesis of N-(7-fluoro-4-hydroxypyrido[3,2-d]pyrimidin-2-yl)acetamide (113a). Acetic anhydride was cooled to 0° C. under nitrogen and 2-amino-7-fluoropyrido[3,2-d]pyrimidin-4-ol 43B (200 mg, 1.11 mmol; Supplied by Medicilon, Shanghai) was added. The reaction mixture was then heated to 110° C. for 4 h. The mixture was cooled and concentrated under reduced pressure. The residue was triturated with DCM (20 mL), and the solids removed by filtration and air dried to provide of compound 113a as a solid. LCMS-ESI⁺ (m/z): [M+H]⁺ calculated for C₉H₇FN₄O₂: 223.06; found: 222.96; t_{R}=0.58 min.

Synthesis of N⁴-(tert-butyl)-7-methoxypyrido[3,2-d]pyrimidine-2,4-diamine (113). 113a was suspended in POCl₃ (5 mL) and heated to 110° C. for 1 h. The reaction was then cooled and POCl₃ removed under reduced pressure. The residue was co-evaporated with toluene (15 mL) and then treated within THF (5 mL). tert-Butylamine (70 μL, 0.66 mmol) was added and the mixture stirred at rt for 15 minutes. 25% Sodium methoxide in methanol (100 μL, 0.45 mmol) was added and the reaction mixture heated in a sealed vessel at 80° C. The reaction mixture was allowed to cool to rt and was directly subjected to preparative HPLC (Synergi 4 u Polar-RP 80 A, Axia; 10% aq. acetonitrile-70% aq. acetonitrile with 0.1% TFA, over 20 min. gradient). The product fractions were concentrated in vacuo to afford 113 as its TFA salt. ¹H NMR (400 MHz, Methanol-d4) δ 8.30 (d, J=2.5 Hz, 1H), 8.04 (s, 1H), 7.18 (d, J=2.6 Hz, 1H), 3.99 (s, 3H), 1.61 (s, 9H). ¹⁹F NMR (376 MHz, Methanol-d4) δ −77.51. LCMS-ESI⁺ (m/z): [M+H]⁺ calculated for C₁₂H₁₇N₅O: 248.14; found: 248.09; t_{R}=0.81 min.

### Example 114

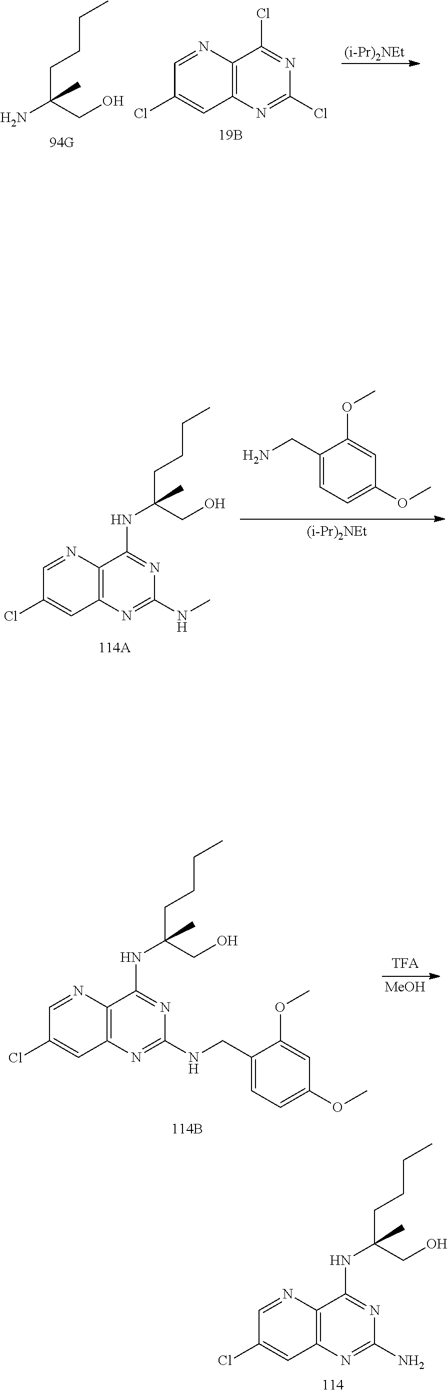

Synthesis of (R)-2-((2,7-dichloropyrido[3,2-d]pyrimidin-4-yl)amino)-2-methylhexan-1-ol (114A). To a solution of 94G (75 mg, 0.30 mmol) and 19B (51 mg, 0.30 mmol) in THF (5 mL) was added N,N-diisopropylethylamine (0.16 mL, 0.90 mmol). After stirring at 80° C. for 23 h, the reaction was cooled to ambient temperature, diluted with EtOAc (50 mL), washed with water (50 mL) and brine (50 mL), dried over Na₂SO₄, then filtered and concentrated in vacuo. The residue was subjected to silica gel chromatography eluting with hexanes-EtOAc (0-75%) to provide 114A. LCMS (m/z): 329.11 [M+H]⁺; t_{R}=1.27 min. on LC/MS Method A.

Synthesis of (R)-2-((7-chloro-2-((2,4-dimethoxybenzyl)amino)pyrido[3,2-d]pyrimidin-4-yl)amino)-2-methylhexan-1-ol (114B). To a solution of 114A in THF (5 mL) was added N,N-diisopropylethylamine (0.16 mL, 0.90 mmol) followed by 2,4-dimethoxybenzylamine (0.25 mL, 1.5 mmol). After stirring at 100° C. for 18 h, the reaction was cooled to ambient temperature, diluted with EtOAc (100 mL), washed with water (100 mL) and brine (100 mL), dried over Na₂SO₄, then filtered and concentrated in vacuo. The residue was subjected to silica gel chromatography eluting with hexanes-EtOAc (15-100%) to provide 114B. LCMS (m/z): 460.29 [M+H]⁺; t_{R}=0.94 min. on LC/MS Method A.

Synthesis of (R)-2-((2-amino-7-chloropyrido[3,2-d]pyrimidin-4-yl)amino)-2-methylhexan-1-ol (114). To 114B (11 mg, 0.02 mmol) was added TFA (3 mL). After 4 h, the reaction mixture was concentrated in vacuo and coevaporated with MeOH (3×20 mL). The residue was suspended in MeOH (20 mL) and filtered. After stirring overnight, the solution was concentrated in vacuo to afford 114 as a TFA salt. LCMS (m/z): 310.12 [M+H]⁺; t_{R}=0.98 min. on LC/MS Method A. ¹H NMR (400 MHz, Methanol-d₄) δ 8.59 (d, J=2.1 Hz, 1H), 8.25 (s, 1H), 7.91 (d, J=2.1 Hz, 1H), 3.97 (d, J=11.3 Hz, 1H), 3.71 (d, J=11.2 Hz, 1H), 2.10 (ddd, J=13.9, 10.9, 5.0 Hz, 1H), 1.96-1.82 (m, 1H), 1.54 (s, 3H), 1.35 (qt, J=6.8, 2.8 Hz, 4H), 0.95-0.88 (m, 3H). ¹⁹F NMR (377 MHz, Methanol-d₄) δ −77.61.

### Example 115

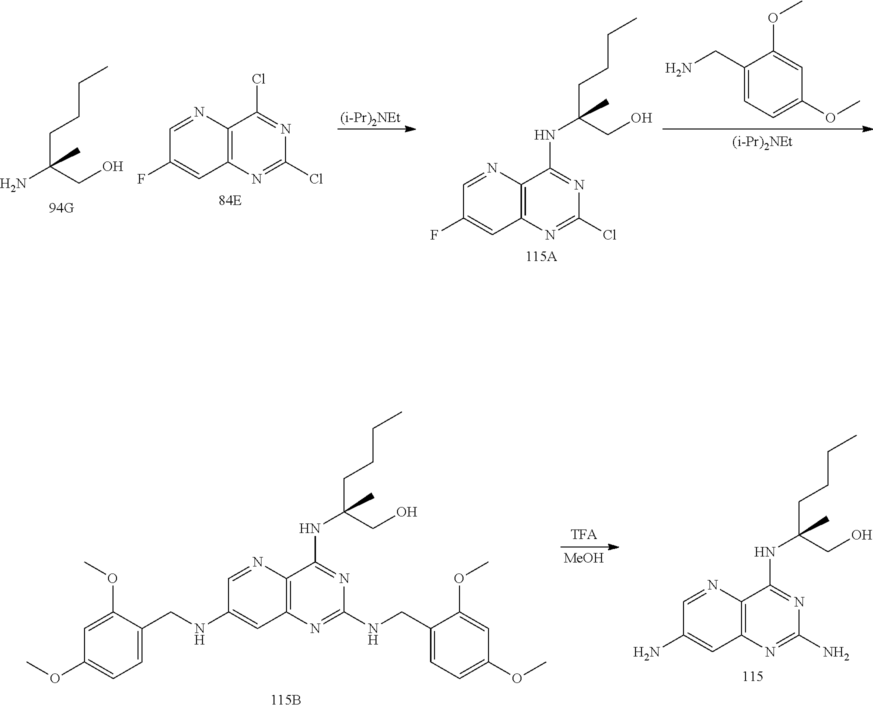

Synthesis of (R)-2-((2-chloro-7-fluoropyrido[3,2-d]pyrimidin-4-yl)amino)-2-methylhexan-1-ol (115A). To a solution of 94G (55 mg, 0.30 mmol) and 84E (65 mg, 0.30 mmol) in THF (5 mL) was added N,N-diisopropylethylamine (0.16 mL, 0.90 mmol). After stirring at 80° C. for 18 h, the reaction was cooled to ambient temperature, diluted with EtOAc (50 mL), washed with water (50 mL) and brine (50 mL), dried over Na₂SO₄, then filtered and concentrated in vacuo. The residue was subjected to silica gel chromatography eluting with hexanes-EtOAc to provide 115A. LCMS (m/z): 313.08 [M+H]⁺; t_{R}=1.19 min. on LC/MS Method A.

Synthesis of (R)-2-((2,7-bis((2,4-dimethoxybenzyl)amino)pyrido[3,2-d]pyrimidin-4-yl)amino)-2-methylhexan-1-ol (115B). To a solution of 115A in THF (5 mL) was added N,N-diisopropylethylamine (0.16 mL, 0.90 mmol) followed by 2,4-dimethoxybenzylamine (0.25 mL, 1.5 mmol). After stirring at 140° C. for 18 h, the reaction was cooled to ambient temperature, diluted with EtOAc (100 mL), washed with water (100 mL) and brine (100 mL), dried over Na₂SO₄, then filtered and concentrated in vacuo. The residue was subjected to silica gel chromatography eluting with hexanes-EtOAc (0-100%) to provide 115B. LCMS (m/z): 444.23 [M+H]⁺; t_{R}=0.90 min. on LC/MS Method A.

Synthesis of (R)-2-((2,7-diaminopyrido[3,2-d]pyrimidin-4-yl)amino)-2-methylhexan-1-ol (115). To 115B (14 mg, 0.02 mmol) was added TFA (3 mL). After 4 h, the reaction mixture was concentrated in vacuo and coevaporated with MeOH (3×20 mL). The residue was suspended in MeOH (20 mL) and filtered. After stirring overnight, the solution was concentrated in vacuo to fford 115 as a bis-TFA salt. LCMS (mi/z): 291.19 [M+H]⁺; t_{R}=0.93 min. on LC/MS Method A. ¹H NMR (400 MHz, Methanol-d₄) δ 8.02 (d, J=2.4 Hz, 1H), 6.69 (d, J=2.4 Hz, 1H), 3.94 (d, J=11.2 Hz, 1H), 3.69 (d, J=11.2 Hz, 1H), 2.06 (ddd, J=13.4, 11.0, 5.0 Hz, 1H), 1.91-1.79 (m, 1H), 1.49 (s, 3H), 1.35 (td, J=7.4, 4.2 Hz, 4H), 0.92 (t, J=7.0 Hz, 3H). ¹⁹F NMR (377 MHz, Methanol-d₄) δ −77.58.

### Example 116

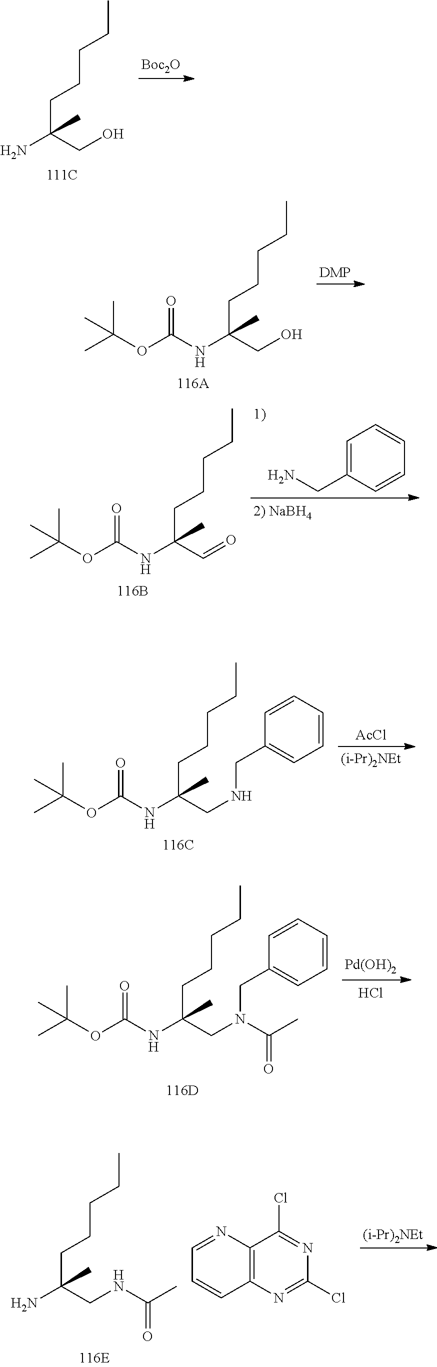

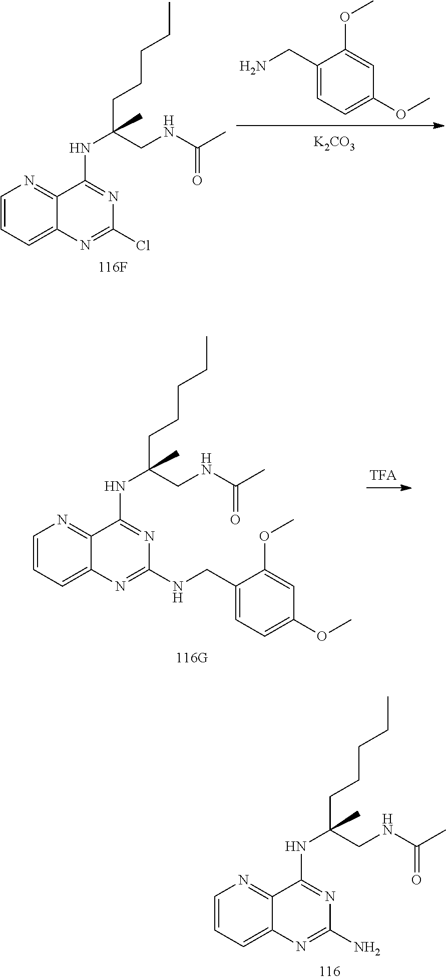

Synthesis of tert-butyl (R)-(1-hydroxy-2-methylheptan-2-yl)carbamate (116A). To 111C (315 mg, 2.17 mmol) in THF (30 mL) was added 1M aqueous NaOH (2.2 mL) followed by DIPEA (1.7 mL, 9.76 mmol) and Boc₂O (2.17 g, 9.94 mmol). After 18 hours, the mixture was diluted with water (50 mL) and extracted with EtOAc (2×50 mL). The combined organics were washed with brine (100 mL), dried over Na₂SO₄, and concentrated in vacuo. The material was purified by flash chromatography equipped with an ELSD using hexane-EtOAc (0-50%) to afford 116A. LCMS (m/z): 245.77 [M+H]⁺; t_{R}=1.15 min. on LC/MS Method A.

Synthesis of tert-butyl (R)-(2-methyl-1-oxoheptan-2-yl)carbamate (116B). To a solution of 116A (378 mg, 1.54 mmol) in DCM (15 mL) was added Dess-Martin periodinane (981 g, 2.31 mmol). After 90 min, the reaction was quenched with sat. Na₂S₂O_{3(aq)} (20 mL). The layers were separated and the aqueous was extracted with DCM (25 mL). The combined organics were washed with water (50 mL) and brine (50 mL), dried over Na₂SO₄, and concentrated in vacuo. The material was purified by flash chromatography equipped with an ELSD using hexane-EtOAc (0-50%) to afford 116B. LCMS (m/z): 143.95 [M+H]⁺; t_{R}=1.23 min. on LC/MS Method A.

Synthesis of tert-butyl (R)-(1-(benzylamino)-2-methylheptan-2-yl)carbamate (116C). To a solution of 116B (351 mg, 1.44 mmol) in MeOH (6 mL) was added benzylamine (0.16 mL, 1.44 mmol). After 18 h, sodium borohydride (91 mg, 2.17 mmol) was added to the reaction. After 90 min, the mixture was concentrated in vacuo. The residue was diluted with EtOAc (25 mL), washed with 1 M NaOH_{(aq)} (20 mL), dried over Na₂SO₄, and concentrated in vacuo to provide crude 116C that was used without further purification. LCMS (m/z): 335.02 [M+H]⁺; t_{R}=0.95 min. on LC/MS Method A.

Synthesis of tert-butyl (R)-(1-(N-benzylacetamido)-2-methylheptan-2-yl)carbamate (116D). To a solution of 116C (519 mg, 1.55 mmol) in THF (15 mL) was added N,N-diisopropylethylamine (0.54 mL, 3.10 mmol) followed by acetyl chloride (0.17 mL, 2.33 mmol). After 60 min, the reaction was diluted with EtOAc (50 mL), washed with water (30 mL), sat. NaHCO_{3(aq)} (30 mL), and brine (30 mL), dried over Na₂SO₄, and concentrated in vacuo. The material was purified by flash chromatography equipped with an ELSD using hexane-EtOAc (0-100%) to afford 116D. LCMS (m/z): 376.82 [M+H]⁺; t_{R}=1.36 min. on LC/MS Method A.

Synthesis of (R)—N-(2-amino-2-methylheptyl)acetamide (116E). To a solution of 116D (584 mg, 1.55 mmol) in EtOH (15 mL) was added HCl solution (0.78 mL, 3.10 mmol, 4 M in 2,4-dioxane). The solution was then purged with Ar and Pd(OH)₂ (441 mg) were added. The mixture was purged with H₂ and heated to 75° C. After 18 h, the mixture was cooled to ambient temperature, purged with Ar, filtered, and concentrated in vacuo to provide crude 116E (288 mg) as an HCl salt. LCMS (m/z): 186.96 [M+H]⁺; t_{R}=0.52 min. on LC/MS Method A.

Synthesis of (R)—N-(2-((2-chloropyrido[3,2-d]pyrimidin-4-yl)amino)-2-methylheptyl)acetamide (116F). To a solution of 116E (50 mg, 0.22 mmol) and 2,4-dichloropyrido[3,2-d]pyrimidine (45 mg, 0.22 mmol) in THF (3 mL) was added N,N-diisopropylethylamine (0.12 mL, 0.67 mmol). After stirring at 80° C. for 18 h, the reaction was cooled to ambient temperature, diluted with EtOAc (25 mL), washed with water (25 mL) and brine (25 mL), dried over Na₂SO₄, and concentrated in vacuo. The residue was subjected to silica gel chromatography eluting with hexanes-EtOAc (0-100%) to provide 116F. LCMS (m/z): 350.06 [M+H]⁺; t_{R}=1.09 min. on LC/MS Method A.

Synthesis of (R)—N-(2-((2-((2,4-dimethoxybenzyl)amino)pyrido[3,2-d]pyrimidin-4-yl)amino)-2-methylheptyl)acetamide (116G). To a solution of 116F (58 mg, 0.17 mmol) in 2-MeTHF (3 mL) was added potassium carbonate (46 mg, 0.33 mmol) followed by 2,4-dimethoxybenzylamine (0.05 mL, 0.33 mmol). After stirring at 85° C. for 18 h, the reaction was cooled to ambient temperature, diluted with EtOAc (25 mL), washed with water (20 mL) and brine (20 mL), dried over Na₂SO₄, then filtered and concentrated in vacuo. The residue was subjected to silica gel chromatography eluting with hexanes-EtOAc (20-100%) to provide 116G. LCMS (m/z): 481.27 [M+H]⁺; t_{R}=0.94 min. on LC/MS Method A.

Synthesis of (R)—N-(2-((2-aminopyrido[3,2-d]pyrimidin-4-yl)amino)-2-methylheptyl)acetamide (116). To 116G (53 mg, 0.11 mmol) was added TFA (3 mL). After 2 h, the reaction mixture was concentrated in vacuo and coevaporated with MeOH (3×20 mL). The residue was suspended in MeOH and filtered. The solution was concentrated in vacuo to afford 116 as a TFA salt. LCMS (m/z): 331.25 [M+H]⁺; t_{R}=0.72 min. on LC/MS Method A. ¹H NMR (400 MHz, Methanol-d₄) δ 8.63 (dd, J=4.4, 1.4 Hz, 1H), 7.85 (dd, J=8.5, 1.4 Hz, 1H), 7.76 (ddd, J=8.5, 4.4, 1.2 Hz, 1H), 3.95 (d, J=14.0 Hz, 1H), 3.56 (d, J=13.9 Hz, 1H), 2.22-2.12 (m, 1H), 1.95 (s, 3H), 1.94-1.85 (m, 1H), 1.54 (s, 3H), 1.41-1.30 (m, 6H), 0.88 (t, J=6.3 Hz, 3H). ¹⁹F NMR (377 MHz, Methanol-d₄) δ −77.86.

### Example 117

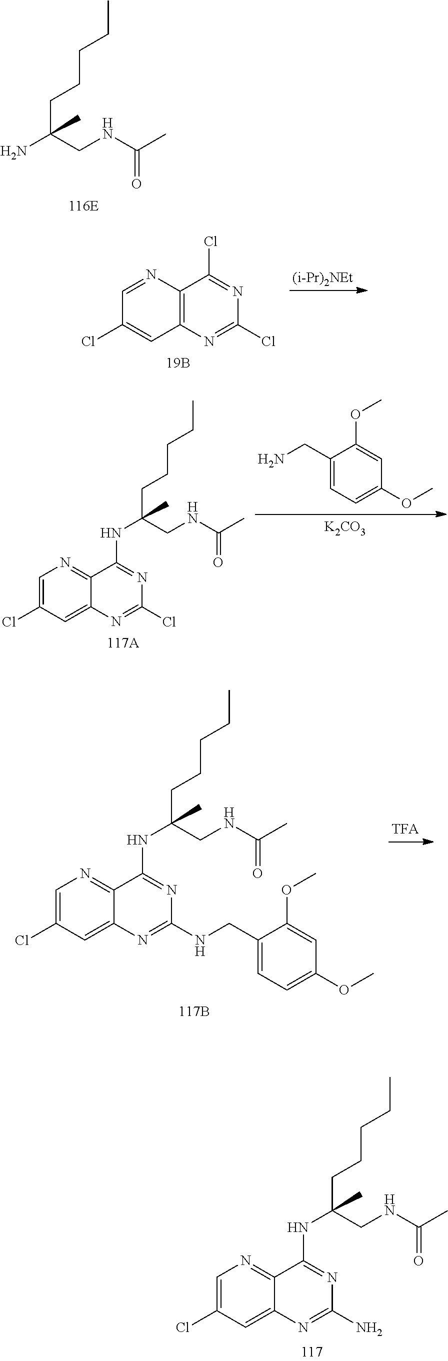

Synthesis of (R)—N-(2-((2,7-dichloropyrido[3,2-d]pyrimidin-4-yl)amino)-2-methylheptyl)acetamide (117A). To a solution of 116E (50 mg, 0.22 mmol) and 19B (53 mg, 0.22 mmol) in THF (3 mL) was added N,N-diisopropylethylamine (0.12 mL, 0.67 mmol). After stirring at 80° C. for 18 h, the reaction was cooled to ambient temperature, diluted with EtOAc (25 mL), washed with water (25 mL) and brine (25 mL), dried over Na₂SO₄, then filtered and concentrated in vacuo. The residue was subjected to silica gel chromatography eluting with hexanes-EtOAc (0-100%) to provide 117A. LCMS (m/z): 384.01 [M+H]⁺; t_{R}=1.77 min. on LC/MS Method A.

Synthesis of (R)—N-(2-((7-chloro-2-((2,4-dimethoxybenzyl)amino)pyrido[3,2-d]pyrimidin-4-yl)amino)-2-methylheptyl)acetamide (117B). To a solution of 117A (33 mg, 0.09 mmol) in 2-MeTHF (3 mL) was added potassium carbonate (24 mg, 0.17 mmol) followed by 2,4-dimethoxybenzylamine (0.05 mL, 0.17 mmol). After stirring at 85° C. for 18 h, the reaction was cooled to ambient temperature, diluted with EtOAc (50 mL), washed with water (20 mL) and brine (20 mL), dried over Na₂SO₄, then filtered and concentrated in vacuo. The residue was subjected to silica gel chromatography eluting with hexanes-EtOAc (0-100%) then EtOAc-MeOH (0-25%) to provide 117B. LCMS (m/z): 515.26 [M+H]⁺; t_{R}=1.06 min. on LC/MS Method A.

Synthesis of (R)—N-(2-((2-amino-7-chloropyrido[3,2-d]pyrimidin-4-yl)amino)-2-methylheptyl)acetamide (117). To 117B (38 mg, 0.07 mmol) was added TFA (3 mL). After 2 h, the reaction mixture was concentrated in vacuo and coevaporated with MeOH (3×20 mL). The residue was suspended in MeOH and filtered. The solution was concentrated in vacuo to afford 117 as a TFA salt. LCMS (m/z): 632.22 [M+H]⁺; t_{R}=0.89 min. on LC/MS Method A. ¹H NMR (400 MHz, Methanol-d₄) δ 8.59 (dd, J=3.5, 2.1 Hz, 1H), 7.92 (d, J=1.9 Hz, 1H), 3.93 (d, J=14.0 Hz, 1H), 3.51 (d, J=14.0 Hz, 1H), 2.21-2.10 (m, 1H), 1.96 (s, 3H), 1.95-1.87 (m, 1H), 1.54 (s, 3H), 1.35 (dd, J=17.6, 5.4 Hz, 6H), 0.88 (t, J=6.4 Hz, 3H). ¹⁹F NMR (377 MHz, Methanol-d₄) δ −77.80.

### Example 118

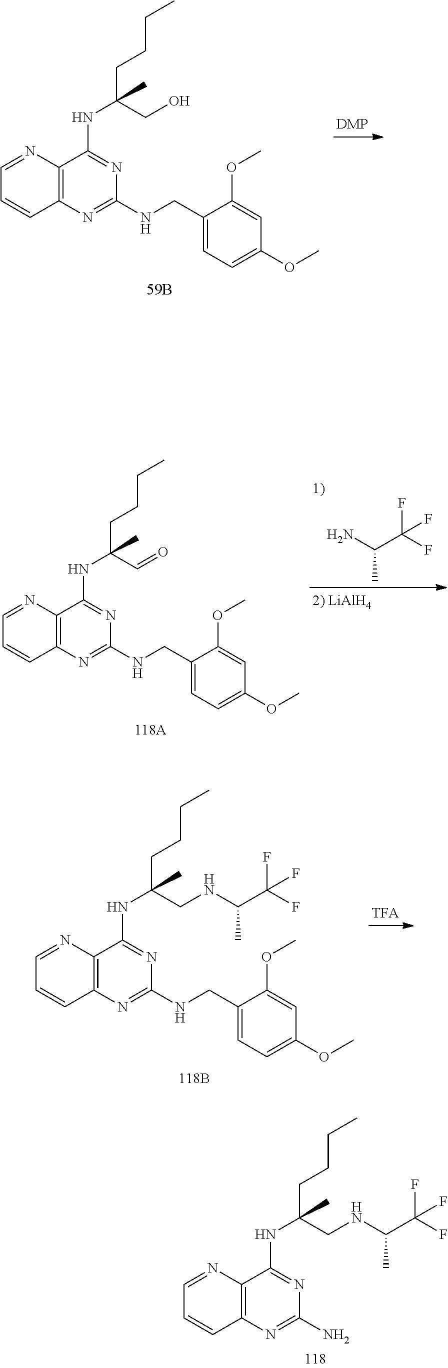

Synthesis of (R)-2-((2-((2,4-dimethoxybenzyl)amino)pyrido[3,2-d]pyrimidin-4-yl)amino)-2-methylhexanal (118A). To a solution of 59B (548 mg, 1.29 mmol) in DCM (24 mL) was added Dess-Martin periodinane (829 mg, 1.93 mmol). After 60 min, the reaction was quenched with sat. Na₂S₂O_{3(aq)} (20 mL), the layers were separated, and the aqueous was extract with DCM (25 mL). The combined organics were washed with water (50 mL), sat. NaHCO_{3(aq)} (50 mL), and brine (50 mL), dried over Na₂SO₄, and concentrated in vacuo. The material was purified by flash chromatography using hexane-EtOAc (25-100%) followed by EtOAc-MeOH (0-20%) to afford 118A. LCMS (m/z): 424.18 [M+H]⁺; t_{R}=1.04 min. on LC/MS Method A.

Synthesis of N²-(2,4-dimethoxybenzyl)-N⁴—((R)-2-methyl-1-(((S)-1,1,1-trifluoropropan-2-yl)amino)hexan-2-yl)pyrido[3,2-d]pyrimidine-2,4-diamine (118B). To a solution of 118A (70 mg, 0.17 mmol) in MeOH (1 mL) was added (S)-1,1,1-trifluoro-2-propylamine (39 mg, 0.33 mmol, supplied by Oakwood Chemical). After 5 h, the reaction was concentrated in vacuo. The residue was diluted with THF (2 mL) and lithium aluminum hydride solution (0.82 mL, 0.82 mmol, 1 M in THF) was added. After 30 min, the reaction was quenched with water (20 mL) and extracted with EtOAc (2×20 mL). The combined organics were dried over Na₂SO₄ and concentrated in vacuo to afford crude 118B. LCMS (m/z): 521.24 [M+H]⁺; t_{R}=1.26 min. on LC/MS Method A.

Synthesis of N⁴—((R)-2-methyl-1-(((S)-1,1,1-trifluoropropan-2-yl)amino)hexan-2-yl)pyrido[3,2-d]pyrimidine-2,4-diamine (118). To 118B (66 mg, 0.13 mmol) was added TFA (3 mL). After 4 h, the reaction mixture was concentrated in vacuo. The residue was suspended in 50% EtOH_{(aq)} (6 mL) and filtered. The solution was purified by preparative HPLC (Synergi 4 u Polar-RP 80 A, Axia; 20% aq. acetonitrile-60% aq. acetonitrile with 0.1% TFA, over 20 min. gradient) to afford 122 as a bis-TFA salt. LCMS (m/z): 371.10 [M+H]⁺; t_{R}=1.14 min. on LC/MS Method A. ¹H NMR (400 MHz, Methanol-d₄) δ 8.62 (dd, J=4.4, 1.4 Hz, 1H), 7.87 (dd, J=8.5, 1.4 Hz, 1H), 7.78 (dd, J=8.5, 4.4 Hz, 1H), 3.75 (hept, J=7.1 Hz, 1H), 3.64 (d, J=12.8 Hz, 1H), 3.28 (d, J=12.8 Hz, 1H), 2.17 (ddd, J=13.6, 11.4, 4.6 Hz, 1H), 1.95 (ddd, J=16.1, 12.3, 4.1 Hz, 1H), 1.61 (s, 3H), 1.42 (d, J=6.9 Hz, 3H), 1.40-1.26 (m, 4H), 0.92 (t, J=6.9 Hz, 3H). ¹⁹F NMR (376 MHz, Methanol-d₄) δ −76.47 (d, J=7.1 Hz), −77.87.

Unless otherwise stated, LC/MS retention times (t_{R}) reported above were measured using LC/MS Method A.

Method for LC/MS HPLC (Method A): HPLC LC/MS chromatograms were generated using a Thermo Scientific LCQ LC/MS system eluting with a Kinetex 2.6u C18100 A, 5×30 mm HPLC column, using a 1.85 minute gradient elution from 2% aq. acetonitrile-98% aq. acetonitrile with 0.1% formic acid modifier.

Method for LC/MS HPLC (Method B): HPLC LC/MS chromatograms were generated using a Thermo Scientific LCQ LC/MS system eluting with a Kinetex 2.6u C18100 A, 5×30 mm HPLC column, using a 2.85 minute gradient elution from 2% aq. acetonitrile-98% aq. acetonitrile with 0.1% formic acid modifier.

### Biological Example 1—PBMC IFNα, IL12-p40 and TNFα Assays

Certain compounds disclosed herein we tested according to the procedure described below. Additionally, certain reference compounds were prepared and tested along with the compounds of the present disclosure. For example, the Compound X was prepared in a manner similar to that disclosed in PCT Application Publication No. WO2012/156498 (where the compound is identified as Compound 72). Compound Y was prepared in a manner similar to that disclosed in PCT Application Publication No. WO2015/014815 (where the compound is identified as Compound 6).

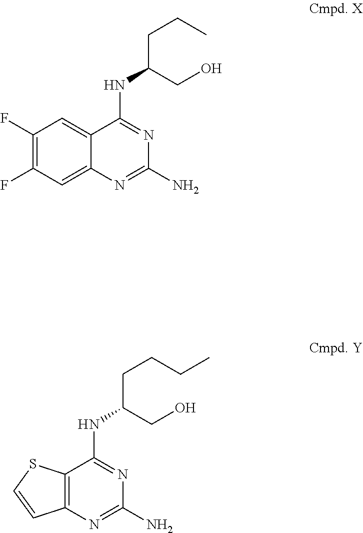 [SMILES: CCC[C@@H](CO)Nc1nc(N)nc2cc(F)c(F)cc12 ; CCCC[C@H](CO)Nc1nc(N)nc2ccsc12]

Compounds were dissolved and stored in DMSO (Sigma-Aldrich, St. Louis, Mo.) at 10 mM concentration.

### Cells and Reagents

Cryopreserved human PBMCs isolated from healthy donors were purchased from StemCell Technologies (Vancouver, Canada). Cell culture medium used was RPMI with L-Glutamine (Mediatech, Manassas, Va.) supplemented with 10% fetal bovine serum (Hyclone, GE Healthcare, Logan, Utah) and Penicillin-Streptomycin (Mediatech). Human TNFα, IL12p40, and IFNα2a 384-well Assay capture plates, standards, buffers and processing reagents were obtained from MesoScale Discovery Technologies (MSD; Rockville, Md.).

Cryopreserved human PBMCs (1×10e8 cells/ml) were thawed at 37° C. and resuspended in 25 mL warm cell culture medium. The cells were pelleted at 200×g (Beckman Avanti J-E) for 5 min and resuspended in 20 mL of fresh culture media. Cells were counted using a Cellometer (Nexcelcom Bioscience), adjusted to 2×10e6 cells, and incubated for 2 hours in an incubator set at 37° C., 5% CO₂ to recover from cryopreservation. Compounds were serially diluted in DMSO at half-log steps to generate a 10-point dose range. Using a Bravo pipette equipped with a 384 well head (Agilent), 0.4 μL of compound was transferred to each well of a 384 well black, clear bottom plate (Greiner Bio-One, Germany) containing 30 μL of cell culture medium. Recovered PBMCs were then dispensed into the assay plate at 50 μL per well (100 k cells/well) using the MicroFlow multichannel dispenser (Biotek). Final DMSO concentration was 0.5%. DMSO was used as the negative control. The plates were incubated for 24 hours at 37° C. PBMCs in the assay plate were pelleted by centrifugation (Beckman Avanti J-E) at 200×g for 5 min.

Using a Biomek FX 384 well pipetting station (Beckman), conditioned culture medium (CCM) from the assay plate was transferred to MSD capture plates customized for each cytokine. For IFNα and IL12-p40 detection, 25 μL and 20 μL of CCM were added directly to each capture plate, respectively. For TNFα detection, CCM was diluted 1:9 in fresh culture medium, and 20 μL of diluted CCM was used. Serially diluted calibration standards for each cytokine were used to generate standard curves and establish assay linearity. The plates were sealed and incubated overnight at 4° C. in a plate shaker (Titer Plate) set at 200 rpm. On the following day, antibodies specific for each cytokine were diluted 1:50 in MSD Diluent 100 antibody dilution buffer. Diluted antibodies were added to corresponding capture plates at 10 μL/well, and incubated at RT for 1-2 hrs in the shaker. The plates were washed with PBST buffer (3×, 60 μl/well) using a Biotek Multiflow plate washer. MSD Read Buffer diluted to 2× in deionized water and 35 μL/well was added via Biomek FX instrument. The plates were read immediately in a MSD6000 reader. Data were normalized to positive and negative controls in each assay plate. AC₅₀ values represent compound concentrations at half-maximal effect based on normalized percent activation and calculated by non-linear regression using Pipeline Pilot software (Accelrys, San Diego, Calif.).

Results of the cytokine profiling assay are reported in Table 1, Table 2, and Table 3 below.

TABLE 1

| | TNFα AC₅₀ | IL12p40 AC₅₀ | IFNα AC₅₀ |
|---|---|---|---|
| Compound | (μM) | (μM) | (μM) |
| 1B | 3.9 | 2 | 2.7 |
| 2B | 5.4 | 2.8 | 4.3 |
| 3B | 29.4 | 15.5 | 14.5 |
| 4B | 9.1 | 5.4 | 5.9 |
| 5B | >50 | >50 | 41 |
| 6B | >50 | >50 | >50 |
| 7 | >50 | >50 | 34 |
| 8 | 20.2 | 19.4 | >50 |
| 9 | 1.9 | 1.1 | 7.2 |
| 10 | 29.2 | 23.8 | >50 |
| 11 | 10.1 | 6 | 6.9 |
| 12 | >50 | >50 | >50 |
| 13 | >50 | >50 | >50 |
| 14 | 1.1 | 0.94 | 1.6 |
| 15 | 1.6 | 1.2 | >200 |
| 16 | >50 | >50 | >50 |
| 17 | >50 | >50 | >50 |
| 18F | 16.1 | 15.2 | 30.6 |
| 19E | 3.3 | 2.5 | 21.6 |
| 20 | 3.2 | 2.8 | 5.4 |
| 21 | 3.1 | 2.3 | 4.8 |
| 22 | >50 | >50 | 35.9 |
| 23C | 24.7 | 25.7 | >50 |
| 24D | 3.4 | 3.1 | 18.4 |
| 25E | 20 | 19.7 | 12.3 |
| 26E | 2.3 | 1.7 | 13.5 |
| 27C | 0.52 | 0.42 | 2 |
| 28 | 28.6 | 28.2 | 45 |
| 29 | 18.3 | 15.5 | 3.9 |
| 30B | 10.6 | 8.6 | >50 |
| 31 | 4.7 | 4.7 | 32.9 |
| 32 | >50 | >50 | >50 |
| 33 | 0.92 | 0.85 | 5.9 |
| 34 | 12.2 | 10.9 | >50 |
| 35 | 39.4 | 22.6 | >50 |
| 36 | 21.5 | 10.8 | >50 |
| 37 | >50 | >50 | >50 |
| 38C | >50 | >50 | >50 |
| 39C | >50 | 41.5 | >50 |
| 40C | 0.94 | 0.87 | 2.4 |
| 41 | 11 | 9.1 | 13 |
| 42B | 1.1 | 0.9 | 3.6 |
| 43C | 1.1 | 1 | 10.9 |
| 44 | 3 | 2.4 | >50 |
| 45 | 1.6 | 1.3 | 8.3 |
| 46C | 28.6 | 28.5 | >50 |
| 47B | 2.70 | 2.0 | >50 |
| 48 | 0.85 | 0.71 | 0.57 |

TABLE 2

| | TNFα AC₅₀ | IL12p40 AC₅₀ | IFNα AC₅₀ |
|---|---|---|---|
| Compound | (μM) | (μM) | (μM) |
| X | 1.2 | 0.97 | 7.1 |
| Y | 11.2 | 13.0 | >50 |

TABLE 3

| | TNFα AC₅₀ | IL12p40 AC₅₀ | IFNα AC₅₀ |
|---|---|---|---|
| Compound | (μM) | (μM) | (μM) |
| 85 | 0.86 | 0.80 | 4.0 |
| 86 | 2.9 | 2.4 | 37.4 |
| 87 | 5.0 | 4.5 | >50 |
| 88 | 0.4 | 0.37 | 35.6 |
| 89 | 2.2 | 1.7 | >50 |
| 90 | 0.86 | 0.62 | 7.8 |
| 91 | 2.0 | 1.9 | >50 |
| 92 | 4.3 | 4.7 | >50 |
| 93 | 0.44 | 0.40 | >50 |
| 94 | 1.0 | 0.7 | 2.2 |
| 95 | 0.15 | 0.15 | >50 |
| 96 | 1.1 | 1.0 | 2.8 |
| 97 | 0.14 | 0.13 | 26.0 |
| 98 | 0.24 | 0.23 | 134 |
| 99 | 3.4 | 3.6 | >50 |
| 100 | 3.8 | 3.5 | 4.6 |
| 101 | 0.10 | 0.11 | >50 |
| 102 | 0.81 | 0.76 | >50 |
| 103 | 3.3 | 2.6 | 10.3 |
| 104 | 2.1 | 1.9 | 4.2 |
| 105 | 3.5 | 3.4 | >50 |
| 106 | 13.8 | 10.2 | >50 |
| 107 | 2.8 | 1.8 | >50 |
| 108 | 38.7 | 22.0 | >50 |
| 110 | 32.6 | 32.6 | 32.6 |
| 111 | 0.61 | 0.47 | 19.8 |
| 112 | 0.36 | 0.33 | >50 |
| 113 | 12.5 | 13.6 | >50 |
| 114 | 0.34 | 0.20 | 34.1 |
| 115 | 0.024 | 0.027 | 9.0 |
| 116 | 0.036 | 0.11 | >50 |
| 117 | 0.37 | 0.33 | >50 |
| 118 | 9.3 | 9.1 | >50 |

In certain embodiments, certain compounds disclosed herein have an AC₅₀ for TNFα that is less than about 100 μM, less than about 50 μM, less than about 40 μM, less than about 30 μM, less than about 25 μM less, than about 20 μM, less than about 15 μM, less than about 10 μM, less than about 5 μM, less than about 4 μM, less than about 3 μM, less than about 2 μM, or less than about 1 μM. In certain embodiments, certain compounds disclosed herein have an AC₅₀ for TNFα that is greater than about 25 μM or greater than about 50 μM. In certain embodiments, certain compounds disclosed herein have an AC₅₀ for TNFα that is less than about 0.75 μM, less than about 0.5 μM, or less than about 0.25 μM. As is understood by those of skill in the art, the induction of TNFα is associated with agonism of TLR8.

In certain embodiments, certain compounds disclosed herein have an AC₅₀ for IL12p40 that is less than about 100 μM, less than about 50 μM, less than about 40 μM, less than about 30 μM, less than about 25 μM, less than about 20 μM, less than about 15 μM, less than about 10 μM, less than about 5 μM less than about 4 μM, less than about 3 μM, less than about 2 μM, less than about 1 μM, or less than about 0.5 μM. In certain embodiments, certain compounds disclosed herein have an AC₅₀ for IL12p40 that is greater than about 25 μM or greater than about 50 μM. As is understood by those of skill in the art, the induction of IL12p40 is associated with agonism of TLR8.

In certain embodiments, certain compounds disclosed herein have an AC₅₀ for IFNα that is less than about 200 μM, less than about 100 μM, less than about 50 μM, less than about 40 μM, less than about 30 μM, less than about 25 μM, less than about 20 μM, less than about 15 μM, less than about 10 μM, less than about 5 μM, less than about 4 μM, less than about 3 μM, less than about 2 μM, or less than about 1 μM. In certain embodiments, certain compounds disclosed herein have an AC₅₀ for IFNα that is greater than about 25 μM, greater than about 50 μM, greater than about 100 μM, greater than about 150 μM, or greater than about 200 μM. As is understood by those of skill in the art, the induction of IFNα is associated with agonism of TLR7.

In certain embodiments, the compounds of the present disclosure are selective TLR8 agonists. Compounds that are selective TLR8 agonists produce a cytokine effect associated with TLR8 induction (e.g. TNFα and IL12p40) at a lower concentration than that associated with TLR7 induction (e.g. IFNα). In certain embodiments, when analyzed in the cytokine profiling assay, the compounds induce IFNα at a concentration at least about 2 times higher than the concentration at which TNFα and/or IL12p40 are induced; in certain embodiments the compounds induce IFNα at a concentration at least about 4 times higher than the concentration at which TNFα and/or IL12p40 are induced; in certain embodiments the compounds induce IFNα at a concentration at least about 6 times higher than the concentration at which TNFα and/or IL12p40 are induced; in certain embodiments the compounds induce IFNα at a concentration at least about 8 times higher than the concentration at which TNFα and/or IL12p40 are induced; in certain embodiments the compounds induce IFNα at a concentration at least about 10 times higher than the concentration at which TNFα and/or IL12p40 are induced; in certain embodiments the compounds induce IFNα at a concentration at least about 20 times higher than the concentration at which TNFα and/or IL12p40 are induced; in certain embodiments the compounds induce IFNα at a concentration at least about 30 times higher than the concentration at which TNFα and/or IL12p40 are induced; in certain embodiments the compounds induce IFNα at a concentration at least about 40 times higher than the concentration at which TNFα and/or IL12p40 are induced; in certain embodiments the compounds induce IFNα at a concentration at least about 50 times higher than the concentration at which TNFα and/or IL12p40 are induced; in certain embodiments the compounds induce IFNα at a concentration at least about 75 times higher than the concentration at which TNFα and/or IL12p40 are induced; in certain embodiments the compounds induce IFNα at a concentration at least about 100 times higher than the concentration at which TNFα and/or IL12p40 are induced; in certain embodiments the compounds induce IFNα at a concentration at least about 125 times higher than the concentration at which TNFα and/or IL12p40 are induced; in certain embodiments the compounds induce IFNα at a concentration at least about 150 times higher than the concentration at which TNFα and/or IL12p40 are induced; in certain embodiments the compounds induce IFNα at a concentration at least about 175 times higher than the concentration at which TNFα and/or IL12p40 are induced; and in certain embodiments the compounds induce IFNα at a concentration at least about 200 times higher than the concentration at which TNFα and/or IL12p40 are induced.

As is understood by those of skill in the art, each compound of the present disclosure may have AC₅₀ values for each cytokine tested (e.g. TNFα, IL12p40, and IFNα) that include various combinations of the ranges disclosed above. As such, the present disclosure provides for such combinations. Further, the ability of any particular compound or group of compounds to selectively modulate a particular receptor can be extrapolated from the AC₅₀ data disclosed herein. One of skill in the art will necessarily appreciate the various selectivities of any particular compound or group of compounds. Biological Example 2—Efficacy study in WHV-infected woodchucks

The in vivo antiviral efficacy of a compound disclosed herein was evaluated in the woodchuck model of CHB. Woodchucks chronically infected with woodchuck hepatitis virus (WHV) (n=23) were stratified into a placebo group (n=11), a 1 mg/kg dose group (n=6), and a 3 mg/kg dose group (n=6) based on gender and baseline antiviral parameters. Animals with high gamma glutamyltransferase (GGT) levels (that correlate with an increased risk of hepatocellular carcinoma (HCC)) and/or with liver tumors observed at the pre-study biopsy screening were included in the placebo group. This stratification was performed so that adverse events (including death) associated with HCC would not confound safety assessment of the dosing groups receiving a compound disclosed herein. The plan for this ongoing study was as follows: animals were dosed PO once a week for 8 weeks with compound or vehicle, followed by a follow-up period of 24 weeks. The animals were monitored for safety and in-life parameters (blood chemistry/hematology/temperature), pharmacokinetics (serum PK), pharmacodynamics (whole blood MARCO mRNA and WHV-specific T cell responses) and antiviral efficacy (serum WHV DNA, woodchuck hepatitis surface antigen (WHsAg) and anti-sAg antibodies, and liver WHV cccDNA, DNA and mRNA).

Interim analysis of this ongoing study revealed that animals dosed with vehicle or 1 mg/kg for 8 weeks did not have any changes in serum WHV DNA or WHsAg levels. In contrast, there was a strong decline in both viral endpoints in 4/6 animals in the 3 mg/kg dose group. Serum WHV DNA and WHsAg levels for three of these animals did not revert at week 12, four weeks after cessation of treatment. Of note, three animals had detectable levels of anti-WHsAg starting at week 4 that were still increasing, stabilizing, or decreasing by week 12. These interim data show that a compound of the present disclosure has antiviral and anti-HBsAg activity as well as the ability to induce anti-HBsAg antibody in vivo in the woodchuck model of CHB.

### Biological Example 3—Off Target Toxicity

To assess potential off-target toxicity of certain compounds disclosed herein, the in vitro cytotoxicity of those compounds was profiled using a panel of 5 cell lines with various tissue origins. Compound cytotoxicity was examined in hepatoma-derived Huh-7 and HepG2 cells, prostate carcinoma-derived PC-3 cells, lymphoma derived MT-4 cells and a normal diploid lung cell line MRC-5. HepG2 and PC-3 cells used were adapted to grow in glucose-free galactose-containing medium. These cells have a relatively higher sensitivity to inhibitors of mitochondrial oxidative phosphorylation compared to the same cells maintained in standard glucose-containing culture medium (Marroquin et al., Toxicol. Sci. 2007; 97 (2):539-47). Cell viability was determined by measuring intracellular ATP levels following five days of continuous incubation with test compounds.

### Cell Cultures

The human hepatoma Huh-7 cell line was obtained from ReBLikon GmbH (Mainz, Germany) {20879}. The MT-4 cell line (HTLV-1 transformed, human T lymphoblastoid cells) was obtained from the NIH AIDS Reagent program (Bathesda, Md.). The human hepatoblastoma cell line HepG2, human prostate carcinoma cell line PC-3, and normal fetal lung derived MRC-5 cells were obtained from the American Type Culture Collection (ATCC, Manassas, Va.).

Huh-7 cells were maintained in Dulbecco's Modified Eagle Medium (DMEM) supplemented with 10% fetal bovine serum (FBS, Hyclone, Logan, Utah), 1% non-essential amino acids (Gibco, Carlsbad, Calif.). PC-3 and HepG2 cells were adapted to grow in 0.2% galactose-containing, glucose-free Dulbecco's Modified Eagle Medium (DMEM) supplemented with 10% fetal bovine serum (FBS, Hyclone, Logan, Utah), 1% non-essential amino acids (Gibco, Carlsbad, Calif.), 1% Pyruvate (Cellgro), 1% Glutamax (Invitrogen, Carlsbad, Calif.). Galactose-adapted cells were maintained in the same culture medium. MRC-5 cells were maintained in Eagle's Minimum Essential Medium (EMEM) supplemented with 10% fetal bovine serum (FBS, Hyclone, Logan, Utah). MT-4 cells were maintained in RPMI-1640 supplemented with 10% fetal bovine serum (FBS, Hyclone, Logan, Utah). All cell culture media were also supplemented with 100 Units/mL penicillin, 100 μg/mL streptomycin (Gibco).

### Cytotoxicity Assays

Using a Biotek uFlow Workstation (Biotek, Winooski, Vt.), 1500 HepG2, 1500 PC-3, 500 Huh7 or 1500 MRC-5 cells in 90 μL of culture media were dispensed into each well of black polystyrene tissue culture-treated 384-well plates. Plated cells were incubated for 24 hours in an incubator at 37° C., 5% CO₂ and 90% humidity. Compound serial dilutions were performed in 100% DMSO in 384-well polypropylene (high recovery) plates on a Biomek FX Workstation (Beckman Coulter, Fullerton, Calif.). After 3-fold serial dilutions, 0.4 μL of compounds were transferred into 384-well plates containing cells using a Velocity 11 system equipped with a Bravo 384-well pipettor. The DMSO concentration in the final assay plates was 0.44% (v/v). Cells were incubated with compound(s) for five days at 37° C. Puromycin (44 μM final concentration) and DMSO (0.44%, v/v) were used as a positive and negative controls, respectively

At the end of the incubation period the cytotoxicity assay was performed as follows: Media from 384-well cell culture plates were aspirated with a Biotek EL405 plate-washer (Biotek) and cells were washed with 100 μL PBS once. Twenty microliters of Cell Titer Glo (Promega, Madison, Wis.) was added to each well of the plates with a Biotek uFlow liquid dispenser. Plates were incubated for 15 minutes at room temperature before luminescence was measured with a Perkin Elmer Envision Plate Reader (Perkin Elmer, Waltham, Mass.).

For the MT-4 cytotoxicity assay, 0.4 μL of serially diluted compounds were added to 40 μl of cell maintenance media in 384-well black, solid bottom plate using a Biomek FX workstation (Beckman Coulter). Two thousand cells in 35 μL were added to each well using a Biotek uFlow Workstation (Biotek). Each assay plate contained 10 μM Puromycin (final concentration) and 0.5% DMSO in RPMI-1640 as positive and negative controls, respectively. Assay plates were incubated for five days at 37° C. in an incubator set at 5% CO2 and 90% humidity. After five days, 22 μL of Cell Titer Glo reagent (Promega) was added to the assay plates with a Biotek uFlow Workstation. Plates were subsequently placed on a Perkin Elmer Envision Plate Reader for five minutes before the luminescence signal was read.

### Data Analysis

CC₅₀ values were defined as the compound concentration that caused a 50% decrease in luminescence signal, and were calculated by non-linear regression using Pipeline Pilot software by applying a four parameter fit equation (Accelrys, San Diego, Calif.). Results are summarized in the table below. Individual CC50 values are listed as μM concentrations.

| | CC50 | CC50 | CC50 | CC50 | |
|---|---|---|---|---|---|
| | GALHEPG2 | GALFC3 | HEH7 | MHCS | CC50 |
| Compound | CTG 50 | CTG 50 | CTG 50 | CTG 50 | (MT4) |
| 15 | 1.23 | 0.69 | 19.70 | 7.60 | 8.62 |
| 44 | 1.07 | 0.52 | 9.64 | 3.72 | 1.97 |
| 50 | 8.88 | 5.20 | 35.39 | 40.31 | 31.40 |
| 98 | 6.70 | 4.53 | 21.90 | 24.42 | 16.17 |
| 51 | 44.44 | 37.23 | 44.44 | 44.44 | 57.14 |
| 102 | 27.42 | 19.08 | 44.44 | 44.44 | 57.14 |
| 61 | 44.44 | 44.44 | 44.44 | 44.44 | 212.39 |
| 62 | 44.44 | 29.74 | 44.44 | 44.44 | 19.23 |
| 93 | 7.73 | 3.63 | 44.44 | 38.89 | 18.55 |
| 64 | 3.56 | 1.99 | 10.36 | 6.97 | 6.79 |
| 101 | 44.44 | 44.44 | 44.44 | 44.44 | 56.6 |
| 65 | 44.44 | 44.44 | 44.44 | 44.44 | 56.6 |
| 95 | 28.06 | 16.76 | 31.34 | 44.44 | 44.11 |
| 97 | 33.01 | 27.51 | 44.44 | 44.44 | 30.0 |
| X | 7.85 | 6.79 | 16.91 | 20.93 | 53.77 |
| Y | 0.19 | 9.18 | 2.31 | 0.93 | 1.42 |

As will be appreciated by one of skill in the art, a high ratio of CC₅₀ from the cytotoxicity assays to AC₅₀ (e.g. of TNFα and/or IL12p40) indicates potential good safety margins in vivo.

All references, including publications, patents, and patent documents are incorporated by reference herein, as though individually incorporated by reference. The present disclosure provides reference to various embodiments and techniques. However, it should be understood that many variations and modifications may be made while remaining within the spirit and scope of the present disclosure.

Aspect 1. A compound of Formula (J):

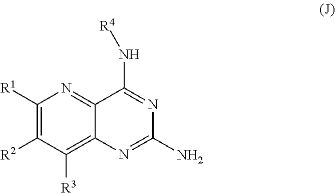

or a pharmaceutically acceptable salt thereof, wherein:

### X is N or CR¹⁰;

R¹ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups;
R² is selected from the group consisting of hydrogen, halogen, C₁₋₆alkyl, CN, —NR^{a}R^{b}, —S(O)₁₋₂R^{a} and OR^{a}, wherein C₁₋₆alkyl is optionally substituted with 1 to 5 R²⁰ groups;
R³ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups;
R⁴ is C₁₋₁₂ alkyl which is optionally substituted with 1 to 5 substituents independently selected from halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆haloalkyl, C₃₋₆cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur;
wherein each C₃₋₆cycloalkyl, 3 to 6 membered heterocyclyl, C₆₋₁₀ aryl, and 5 to 10 membered heteroaryl is optionally substituted with 1 to 5 R²¹ groups;
R¹⁰ is selected from hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups
each R²⁰ is independently selected from the group consisting of halogen, C₁₋₆haloalkyl, CN, —NR^{a}R^{b}, S(O)₁₋₂R^{a}, and OR^{a};
each R²¹ is independently selected from the group consisting of halogen, C₁₋₆alkyl, C₁₋₆haloalkyl, CN, —NR^{a}R^{b}, S(O)₁₋₂R^{a}, and OR^{a}; and
each R^{a} and R^{b} are independently selected from the group consisting of hydrogen and C₁₋₆alkyl; wherein each C₁₋₆alkyl is optionally substituted with 1 to 5 substituents independently selected from halogen, hydroxyl, amino, 5 to 10 membered heteroaryl wherein the 5 to 10 membered heteroaryl has 1 to 3 heteroatoms selected from oxygen, nitrogen, and sulfur, and C₁₋₆haloalkyl; provided that when X is N, R¹ is Cl, R² is H and R³ is H then R⁴ is not CH₂CH₂OMe or CH₂CH₂SO₂Me.
Aspect 2. A compound of Formula (I):

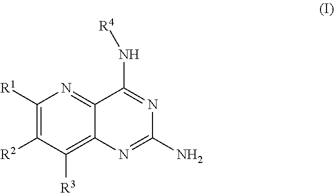

or a pharmaceutically acceptable salt thereof, wherein:
R¹ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups;
R² is selected from the group consisting of hydrogen, halogen, C₁₋₆alkyl, CN, —NR^{a}R^{b}, —S(O)₁₋₂R^{a} and OR^{a}, wherein C₁₋₆alkyl optionally substituted with 1 to 5 R² groups;
R³ is selected from the group consisting of hydrogen, halogen, C₁₋₆alkyl, CN, —NR^{a}R^{b}, —S(O)₁₋₂R^{a}, and OR^{a}, wherein C₁₋₆alkyl is optionally substituted with 1 to 5 R²⁰ groups;
R⁴ is C₁₋₁₂ alkyl which is optionally substituted with 1 to 5 substituents independently selected from halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R, C₁₋₆haloalkyl, C₃₋₆cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur;
wherein each C₃₋₆cycloalkyl, 3 to 6 membered heterocyclyl, C₆₋₁₀ aryl, and 5 to 10 membered heteroaryl is optionally substituted with 1 to 5 R²¹ groups; each R²⁰ is independently selected from the group consisting of halogen, C₁₋₆haloalkyl, CN, —NR^{a}R^{b}, S(O)₁₋₂R^{a}, and OR^{a};
each R²¹ is independently selected from the group consisting of halogen, C₁₋₆alkyl, C₁₋₆haloalkyl, CN, —NR^{a}R^{b}, S(O)₁₋₂R^{a}, and OR^{a}; and
each R^{a} and R^{b} are independently selected from the group consisting of hydrogen and C₁₋₆alkyl, wherein each C₁₋₆alkyl is optionally substituted with 1 to 5 substituents independently selected from halogen, hydroxyl, amino, 5 to 10 membered heteroaryl wherein the 5 to 10 membered heteroaryl has 1 to 3 heteroatoms selected from oxygen, nitrogen, and sulfur, and C₁₋₆haloalkyl; provided that when R¹ is Cl, R² is H and R³ is H then R⁴ is not CH₂CH₂OMe or CH₂CH₂SO₂Me.
Aspect 3. The compound of aspect 1 or 2, or a pharmaceutically acceptable salt thereof, wherein R⁴ is C1-8 alkyl optionally substituted with 1 to 5 substituents independently selected from the group consisting of halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆haloalkyl, C₃₋6cycloalkyl, 3 to 6 membered heterocyclyl wherein the 3 to 6 membered heterocyclyl has 1 to 3 heteroatoms selected from the group consisting of oxygen, nitrogen, and sulfur, C₆₋10 aryl, and 5 to 10 membered heteroaryl wherein the 5 to 10 membered heteroaryl has 1 to 3 heteroatoms selected from the group consisting of oxygen, nitrogen, and sulfur; and wherein each C₃₋₆cycloalkyl, 3 to 6 membered heterocyclyl, C₆₋₁₀ aryl, and 5 to 10 membered heteroaryl is optionally substituted with 1 to 5 R²¹ groups.
Aspect 4. The compound of any of the preceding aspects, or a pharmaceutically acceptable salt thereof, wherein R⁴ is C₁₋₆ alkyl optionally substituted with 1 to 5 substituents independently selected from the group consisting of halogen, —OR^{a}, —C(O)OR^{a}, —C(O)NR^{a}R^{b}, —SR^{a}, C₁₋₆haloalkyl, C₃₋₆cycloalkyl, 3 to 6 membered heterocyclyl, and C₆₋₁₀ aryl; wherein each C₃₋₆cycloalkyl, 3 to 6 membered heterocyclyl, and C₆₋₁₀ aryl is optionally substituted with 1 to 5 R²¹ groups.
Aspect 5. The compound of any of the preceding aspects, or a pharmaceutically acceptable salt thereof, wherein R⁴ is C₁₋₆ alkyl optionally substituted with 1 to 3 substituents independently selected from the group consisting of halogen, —OR^{a}, —C(O)OR^{a}, —C(O)NR^{a}R^{b}, —SR^{a}, —C₁₋₃haloalkyl, C₃₋₆cycloalkyl, 3 to 6 membered heterocyclyl and C₆₋₁₀ aryl; wherein each C₃₋₆cycloalkyl and C₆₋₁₀ aryl is optionally substituted with 1 to 3 R²¹ groups.
Aspect 6. The compound of any of the preceding aspects, or a pharmaceutically acceptable salt thereof, wherein R⁴ is C₁₋₆ alkyl which is optionally substituted with 1 or 2 substituents independently selected from the group consisting of halogen, —OR^{a}, —C(O)OR^{a}, —C(O)NR^{a}R^{b}, —SR^{a}, C₁₋₃haloalkyl, C₃₋₆cycloalkyl, 3 to 6 membered heterocyclyl and C₆₋₁₀ aryl; wherein each C₃₋₆cycloalkyl and C₆₋₁₀ aryl is optionally substituted with 1 to 3 R²⁰ groups and wherein R^{a} and R^{b} are each independently hydrogen or C₁₋₄alkyl, wherein each C₁₋₄ alkyl is optionally substituted with —NH₂, OH, or pyridyl.
Aspect 7. The compound of any of the preceding aspects, or a pharmaceutically acceptable salt thereof, wherein R⁴ is C₁₋₆ alkyl which is optionally substituted with 1 or 2 substituents independently selected from OH, CF₃, —C(O)OH, —C(O)OCH₃, —C(O)NH₂, SCH₃, —C(O)NHCH₃, —C(O)NHCH₂CH₂NH₂, —C(O)NHCH₂CH₂OH, —C(O)NHCH₂-pyridyl, phenyl, tetrahydrofuranyl, and cyclopropyl.
Aspect 8. The compound of any of the preceding aspects, or a pharmaceutically acceptable salt thereof, wherein R⁴ is C₃₋₈ alkyl optionally substituted with 1 to 5 substituents independently selected from the group consisting of halogen, —OR^{a}, —C(O)OR^{a}, —NR^{a}C(O)R^{b}, —SR^{a}, C₁₋₆haloalkyl, C₃₋₆cycloalkyl, 3 to 6 membered heterocyclyl, and C₆₋₁₀ aryl; wherein each C₃₋₆cycloalkyl, 3 to 6 membered heterocyclyl, and C₆₋₁₀ aryl is optionally substituted with 1 to 5 R²¹ groups.
Aspect 9. The compound of any of the preceding aspects, or a pharmaceutically acceptable salt thereof, wherein R⁴ is C₃₋₈ alkyl optionally substituted with 1 to 3 substituents independently selected from the group consisting of halogen, —OR^{a}, —C(O)OR^{a}, —NR^{a}C(O)R^{b}, —SR^{a}, —C₁₋₃haloalkyl, C₃₋₆cycloalkyl, 3 to 6 membered heterocyclyl and C₆₋₁₀ aryl; wherein each C₃₋₆cycloalkyl and C₆₋₁₀ aryl is optionally substituted with 1 to 3 R²¹ groups.
Aspect 10. The compound of any of the preceding aspects, or a pharmaceutically acceptable salt thereof, wherein R⁴ is C₃₋₈ alkyl which is optionally substituted with 1 or 2 substituents independently selected from the group consisting of halogen, —OR^{a}, —C(O)OR^{a}, —NR^{a}C(O)R^{b}, —SR^{a}, C₁₋₃haloalkyl, C₃₋₆cycloalkyl, 3 to 6 membered heterocyclyl and C₆₋₁₀ aryl; wherein each C₃₋₆cycloalkyl and C₆₋₁₀ aryl is optionally substituted with 1 to 3 R²⁰ groups and wherein R^{a} and R^{b} are each independently hydrogen or C₁₋₄alkyl, wherein each C₁₋₄ alkyl is optionally substituted with —NH₂, OH, or pyridyl.
Aspect 11. The compound of any of the preceding aspects, or a pharmaceutically acceptable salt thereof, wherein R⁴ is C₃₋₈ alkyl which is optionally substituted with 1 or 2 substituents independently selected from OH, CF₃, —C(O)OH, —C(O)OCH₃, SCH₃, —NHC(O)CH₃, —NHC(O)CH₂CH₂NH₂, —NHC(O)CH₂CH₂OH, —NHC(O)CH₂-pyridyl, phenyl, tetrahydrofuranyl, and cyclopropyl.
Aspect 12. The compound of any one of the preceeding aspects, or a pharmaceutically acceptable salt thereof, wherein R⁴ is C₃₋₈ alkyl which is optionally substituted with OH.
Aspect 13. The compound of any one of the preceding aspects, or a pharmaceutically acceptable salt thereof, wherein R⁴ has at least one chiral center.
Aspect 14. The compound of aspect 13, or a pharmaceutically acceptable salt thereof, wherein the at least one chiral center is in the S configuration.
Aspect 15. The compound of aspect 13, or a pharmaceutically acceptable salt thereof, wherein the at least one chiral center is in the R configuration.
Aspect 16. The compound of any of the preceding aspects, or a pharmaceutically acceptable salt thereof, wherein R⁴ is selected from the group consisting of:

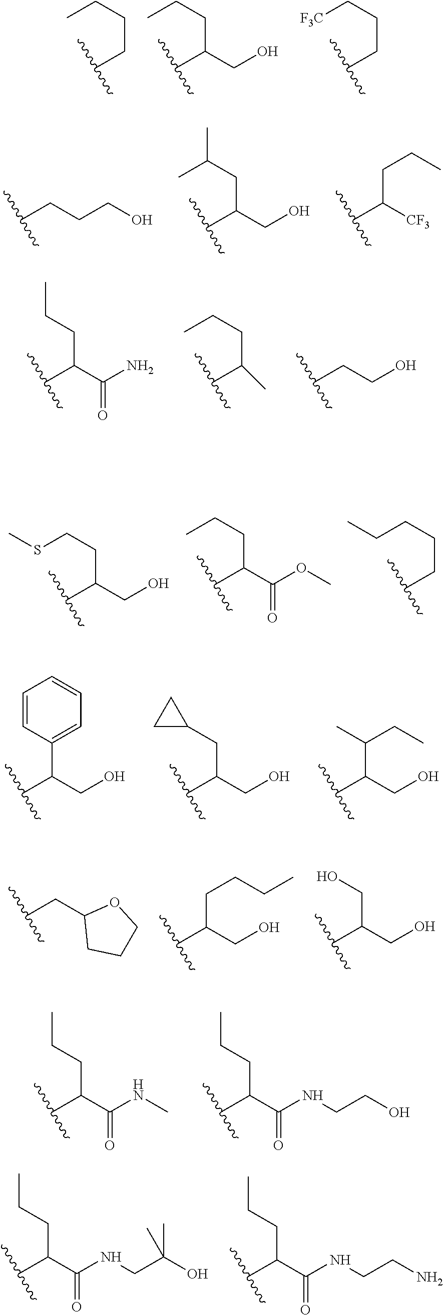

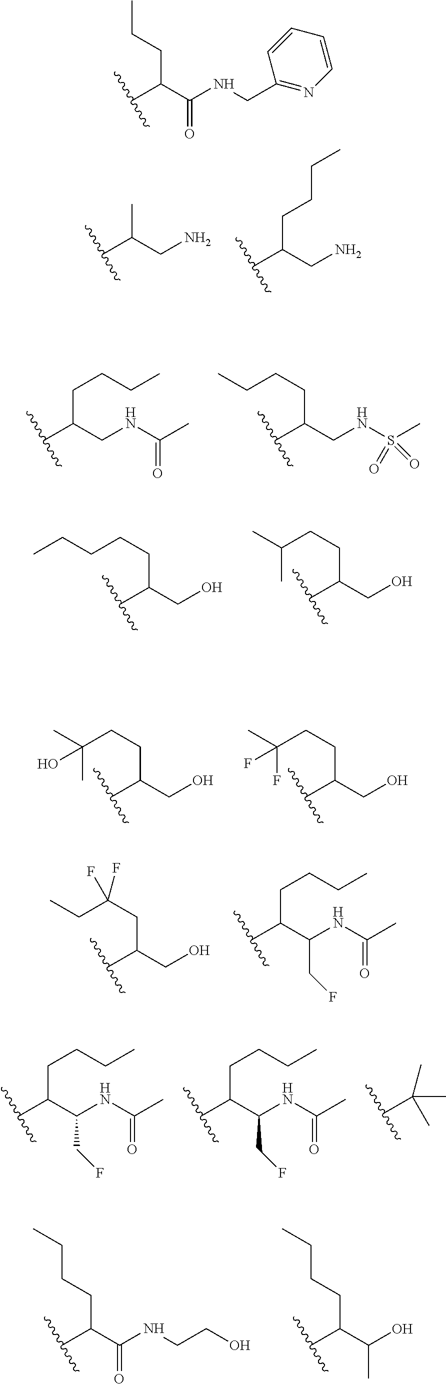

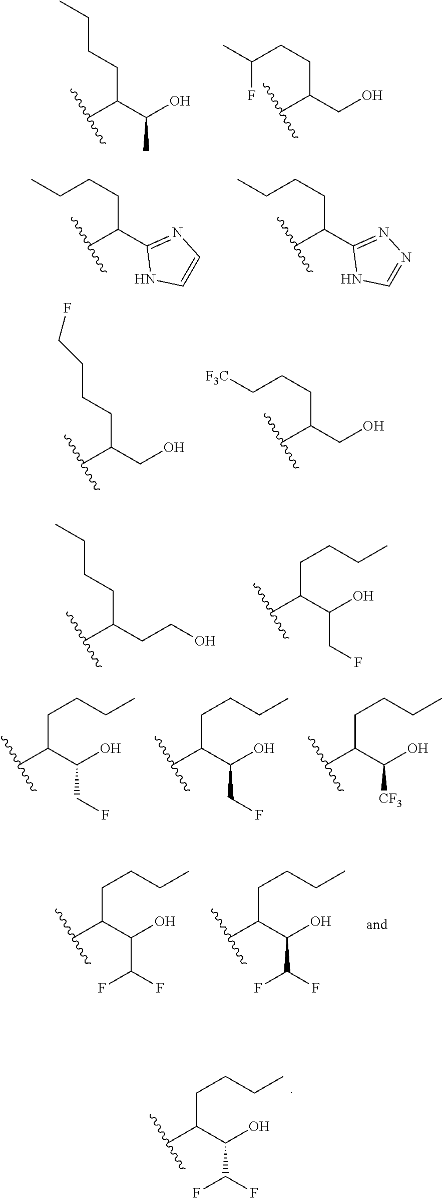

Aspect 17. The compound of any of the preceding aspects, or a pharmaceutically acceptable salt thereof, wherein R⁴ is selected from the group consisting of:

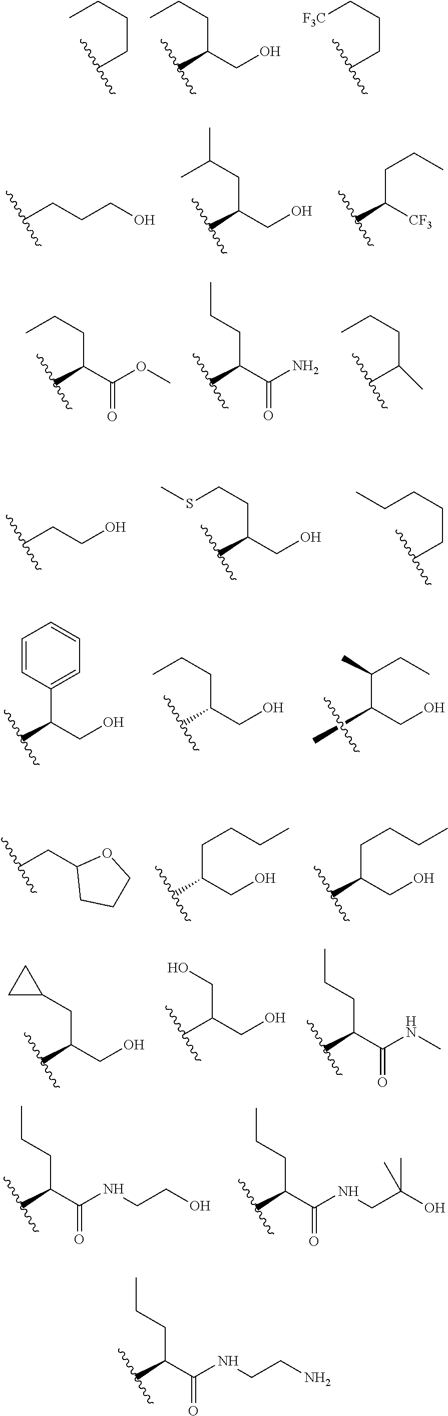

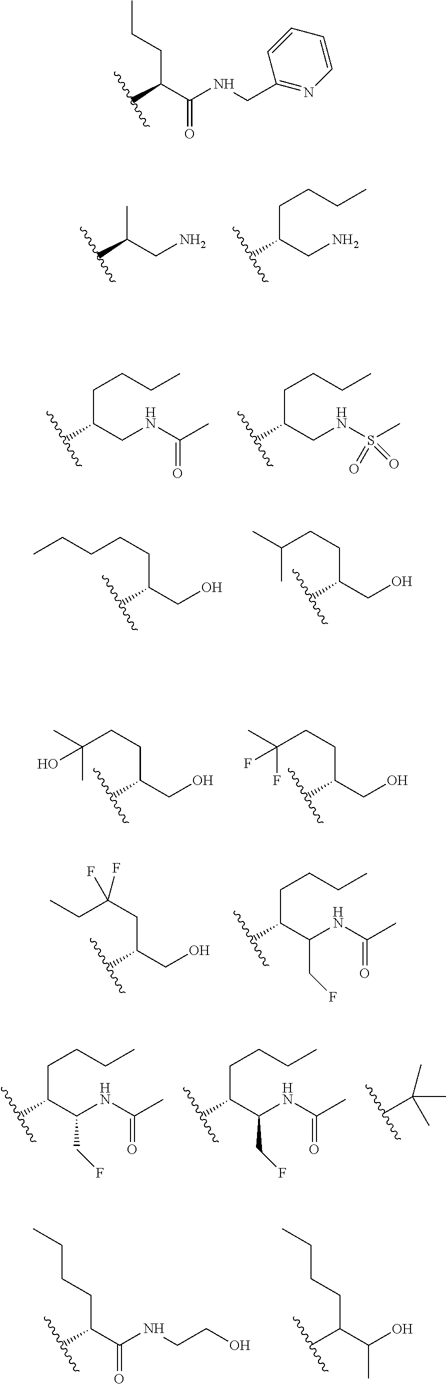

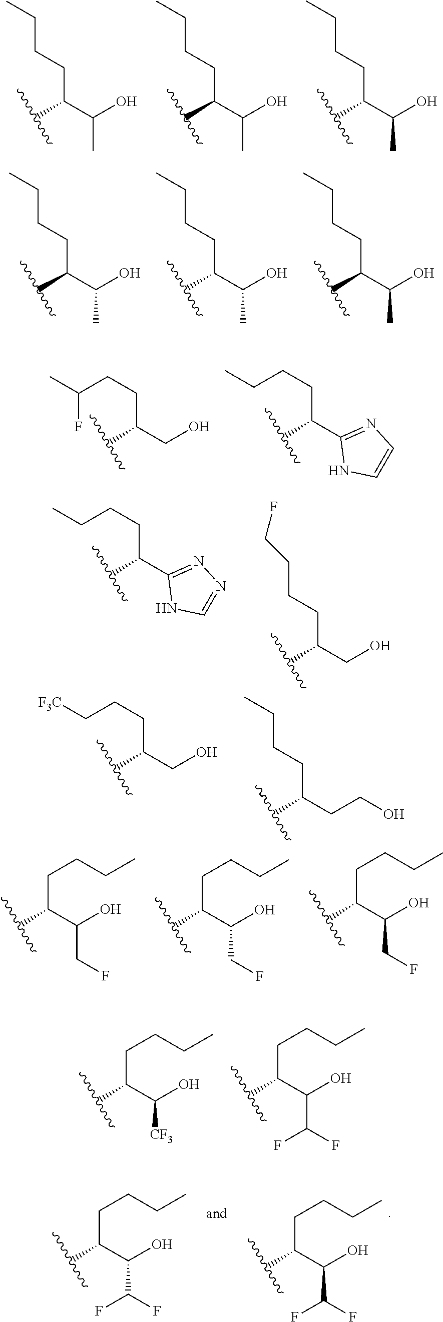

Aspect 18. The compound of any of the preceding aspects, or a pharmaceutically acceptable salt thereof, wherein R⁴ is selected from the group consisting of:

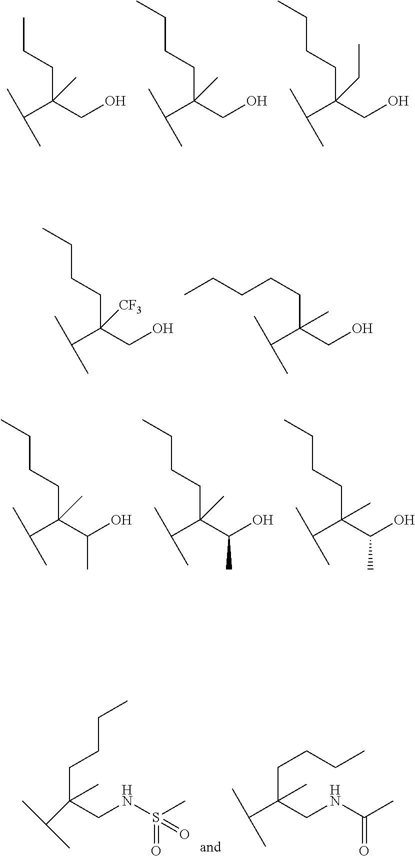

Aspect 19. The compound of any of the preceding aspects, or a pharmaceutically acceptable salt thereof, wherein R⁴ is selected from the group consisting of:

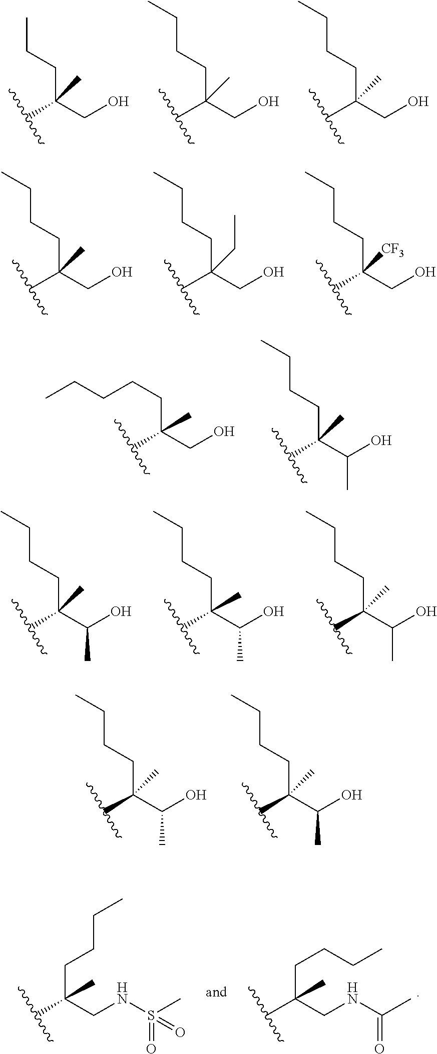

Aspect 20. The compound of any one of aspects 1 to 17, or a pharmaceutically acceptable salt thereof, wherein R⁴ is

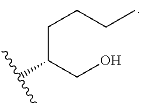 [SMILES: *[C@@H](CO)CCCC]

Aspect 21. The compound of any one of aspects 1 to 3, or a pharmaceutically acceptable salt thereof, wherein the compound is a compound of Formula (II)

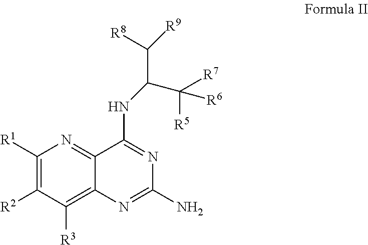

or a pharmaceutically acceptable salt thereof, wherein:
R⁵ is selected from the group consisting of hydrogen, halogen, and methyl; R⁶ is selected from the group consisting of hydrogen, halogen, and methyl; or R⁵ and R⁶ together form an oxo group;
R⁷ is selected from the group consisting of hydrogen, halogen, OR^{a} and NR^{a}R^{b}; R⁸ is selected from the group consisting of hydrogen and methyl;
R⁹ is selected from the group consisting of C₁₋₄ alkyl, C₃₋₅cycloalkyl, and —S—C₁₋4alkyl; and
R^{a} and R^{b} are independently selected from the group consisting of hydrogen and C1-6alkyl;
wherein each C1-6alkyl is optionally substituted with 1 to 3 substituents independently selected from the group consisting of halogen, hydroxyl, pyridyl, and C₁₋₆haloalkyl.
Aspect 22. The compound of any one of aspects 1 to 3 or 21, or a pharmaceutically acceptable salt thereof, wherein the compound is a compound of Formula (Ila)

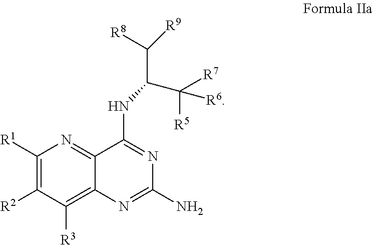

Aspect 23. The compound of aspect 22, or a pharmaceutically acceptable salt thereof, wherein

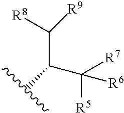

is selected from

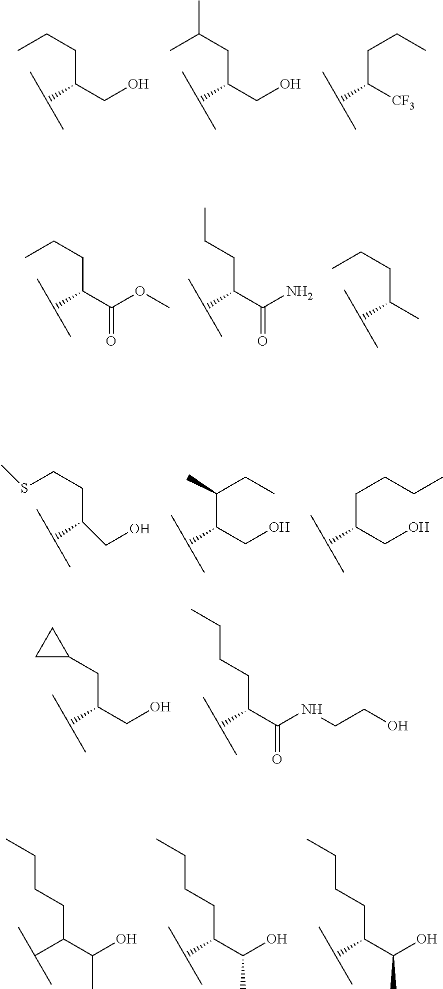

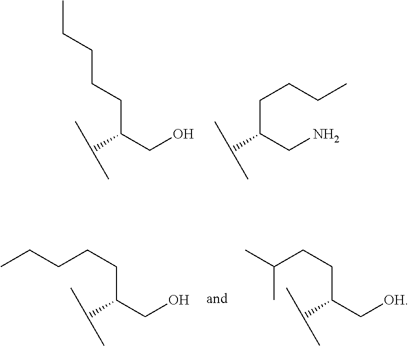

Aspect 24. The compound of any one of aspects 1 to 3 or 21, or a pharmaceutically acceptable salt thereof, wherein the compound is a compound of Formula (IIb)

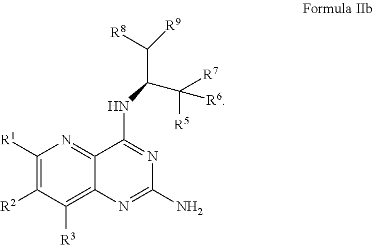

Aspect 25. The compound of aspect 24, or a pharmaceutically acceptable salt thereof, wherein

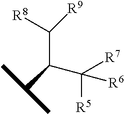

is selected from

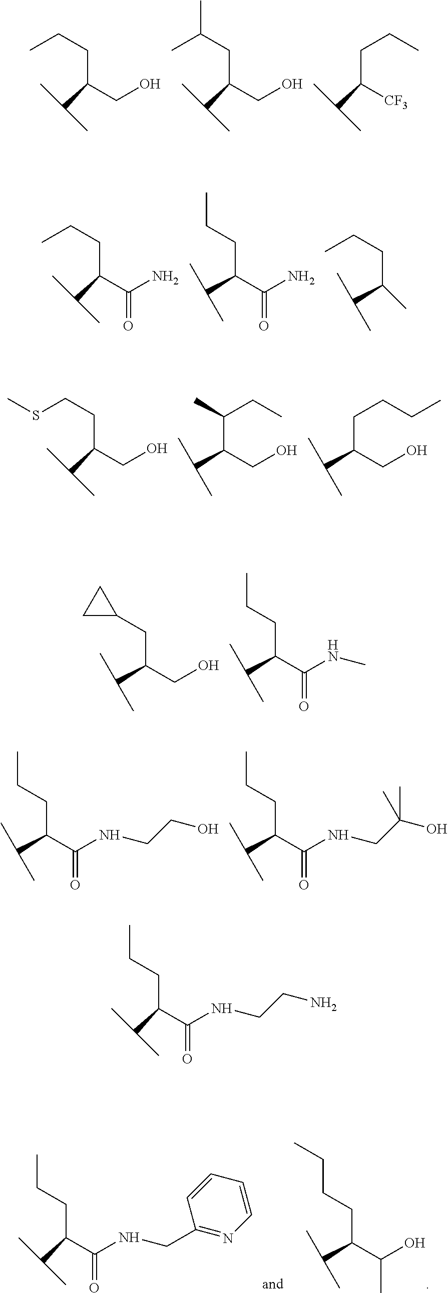

Aspect 26. The compound of any one of aspects 21, 22 and 24, or a pharmaceutically acceptable salt thereof, wherein:
R⁵ is hydrogen;
R⁶ is hydrogen; or R⁵ and R⁶ together form an oxo group;
R⁷ is OR^{a} or NR^{a}R^{b};
R⁸ is hydrogen;
R⁹ is C₁₋₄ alkyl, cyclopropyl or —SCH₃; and
R^{a} and R^{b} are independently selected from the group consisting of hydrogen and C₁₋₄alkyl;
wherein each C₁₋₄alkyl is optionally substituted with 1 to 3 substituents independently selected from halogen, hydroxyl, pyrid-2-yl, and CF₃.
Aspect 27. The compound of any one of aspects 21, 22, 24, or 26, or a pharmaceutically acceptable salt thereof, wherein R⁷ is OH or NH₂.
Aspect 28. The compound of any one of aspects 1 to 15 or 21, or a pharmaceutically acceptable salt thereof, wherein the compound is a compound of Formula (III)

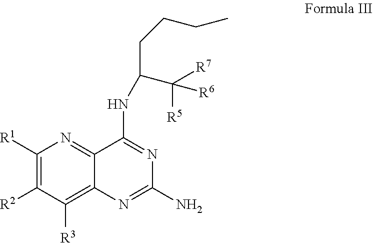

wherein
R⁵ is hydrogen;
R⁶ is hydrogen; or R⁵ and R⁶ together form an oxo group;
R⁷ is selected from the group consisting of OR^{a} and NR^{a}R^{b}; and
R^{a} and R^{b} are independently selected from the group consisting of hydrogen and C₁₋₃alkyl;
wherein each C₁₋₃alkyl is optionally substituted with 1 to 3 substituents independently selected from the group consisting of halogen and hydroxyl.
Aspect 29. The compound of any one of aspects 1 to 15, 21, or 28, or a pharmaceutically acceptable salt thereof, wherein the compound is a compound of Formula (IIIa)

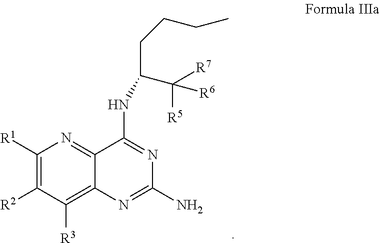

Aspect 30. The compound of any one of aspects 1 to 15, 21, or 28, or a pharmaceutically acceptable salt thereof, wherein the is a compound of Formula (IIIb)

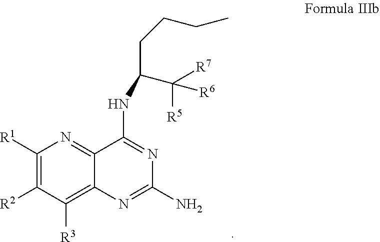

Aspect 31. The compound of any one of aspects 28 to 30, wherein R⁵ and R⁶ are both hydrogen and R⁷ is OR^{a}, wherein R^{a} is hydrogen or C₁₋₃alkyl.
Aspect 32. The compound of any one of aspects 28 to 31, wherein, R⁵ and R⁶ are both hydrogen and R⁷ is OH.
Aspect 33. A compound of Formula (IV),

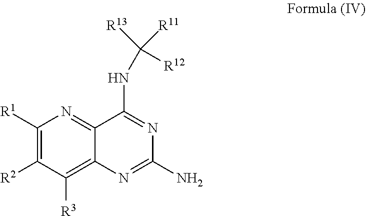

or a pharmaceutically acceptable salt thereof, wherein:
R¹ is selected from the group consisting of hydrogen, halogen, C₁₋₆ alkyl, CN, and OR^{a}, wherein C₁₋₆ alkyl is optionally substituted with 1 to 5 R²⁰ groups;
R² is selected from the group consisting of hydrogen, halogen, C₁₋₆ alkyl, CN, and OR^{a}, wherein C₁₋₆ alkyl optionally substituted with 1 to 5 R²⁰ groups; R³ is selected from the group consisting of hydrogen, halogen, C₁₋₆ alkyl, CN, and OR^{a}, wherein C₁₋₆ alkyl is optionally substituted with 1 to 5 R²⁰ groups;
R¹¹ is selected from the group consisting of hydrogen, C₁₋₂ alkyl, C₃₋₆ cycloalkyl, and C₁₋₃ haloalkyl;
R¹² is selected from C₁₋₃ alkyl, halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, NR^{a}S(O)₂R^{b}, C₁₋₃ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur, wherein the C₁₋₃ alkyl group is optionally substituted with 1 to 5 substituents independently selected from halogen, — OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₃ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur;
R¹³ is selected from C₁₋₆ alkyl, halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur, wherein the C₁₋₆ alkyl is optionally substituted with 1 to 5 substituents independently selected from halogen, —OR^{a}, —NR^{a}R^{b}, CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆ haloalkyl, C₃₋₆ cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur;
each R²⁰ is independently selected from the group consisting of halogen, CN, —NR^{a}R^{b}, and OR^{a}; and
each R^{a} and R^{b} is independently selected from the group consisting of hydrogen and C₁₋₃ alkyl, wherein each C₁₋₃ alkyl is optionally substituted with 1 to 3 substituents independently selected from halogen, hydroxyl, amino, and C₁₋₆ haloalkyl.
Aspect 34. The compound of aspect 33, or a pharmaceutically acceptable salt thereof, wherein the compound is a compound of Formula (IVa)

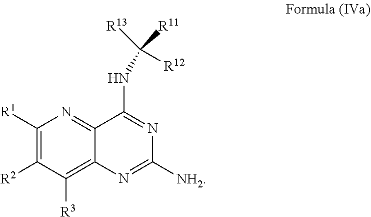

Aspect 35. The compound of aspect 33, or a pharmaceutically acceptable salt thereof, wherein the compound is a compound of Formula (IVb)

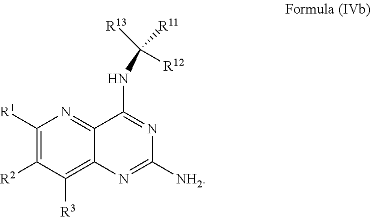

Aspect 36. The compound any one of aspects 33 to 35, or a pharmaceutically acceptable salt thereof, wherein:
R¹ is selected from the group consisting of hydrogen, halogen, and C₁₋₃ alkyl, wherein C₁₋₃ alkyl is optionally substituted with 1 to 5 halogen groups;
R² is selected from the group consisting of hydrogen, halogen, C₁₋₃ alkyl, CN and OR^{a}, wherein C₁₋₃ alkyl is optionally substituted with 1 to 5 halogen groups; and
R³ is selected from the group consisting of hydrogen, halogen, and C₁₋₃ alkyl.
Aspect 37. The compound of any one of aspects 33 to 36, or a pharmaceutically acceptable salt thereof, wherein:
R¹ is selected from the group consisting of hydrogen, methyl, fluoro, chloro, and CF₃;
R² is selected from the group consisting of hydrogen, methyl, ethyl, fluoro, chloro, bromo, CF₃, CN, OH, OMe, and OEt; and
R³ is selected from the group consisting of hydrogen, methyl, fluoro, and chloro.
Aspect 38. The compound of any one of aspects 33 to 37, or a pharmaceutically acceptable salt thereof, wherein:
R¹ is hydrogen;
R² is selected from the group consisting of hydrogen and fluoro; and
R³ is selected from the group consisting of hydrogen and methyl.
Aspect 39. The compound any one of aspects 33 to 38, or a pharmaceutically acceptable salt thereof, wherein R¹¹ is selected from the group consisting of hydrogen, C₁₋₂alkyl and C₁₋₂ haloalkyl.
Aspect 40. The compound of any one of aspects 33 to 39, or a pharmaceutically acceptable salt thereof, wherein R¹¹ is methyl.
Aspect 41. The compound of any one of aspects 33 to 39, or a pharmaceutically acceptable salt thereof, wherein R¹¹ is hydrogen.
Aspect 42. The compound any one of aspects 33 to 41, or a pharmaceutically acceptable salt thereof, wherein
R¹² is selected from the group consisting of C₁₋₂ alkyl, —C(O)NR^{a}R^{b}, and 5 membered heteroaryl having 1 to 3 nitrogen heteroatoms, wherein C₁₋₂ alkyl is optionally substituted with 1 to 5 substituents independently selected from halogen, —OH, —NR^{a}R^{b}, —NR^{a}C(O)R^{b}, —NR^{a}S(O)₂R^{b}, and C₁₋₃ haloalkyl; and
each R^{a} and R^{b} is independently selected from the group consisting of hydrogen and C₁₋₃ alkyl, wherein each C₁₋₃ alkyl is optionally substituted with 1 to 3 substituents independently selected from hydroxyl and amino.
Aspect 43. The compound any one of aspects 33 to 42, or a pharmaceutically acceptable salt thereof, wherein R¹² is C₁₋₂ alkyl, optionally substituted with 1 to 3 substituents independently selected from halogen, —OH, —NH₂, —NHC(O)—C₁₋₃ alkyl, —NHS(O)₂—C₁₋₃ alkyl, and C₁₋₃ haloalkyl.
Aspect 44. The compound any one of aspects 33 to 43, or a pharmaceutically acceptable salt thereof, wherein R¹² is methyl or ethyl, each optionally substituted with —OH or —NHC(O)CH₃.
Aspect 45. The compound of any one of aspects 33 to 42, or a pharmaceutically acceptable salt thereof, wherein R¹² is selected from the group consisting of CH₂OH, CH₂CH₂OH, CH(Me)OH, CH(CH₂F)OH, CH(CHF₂)OH, CH(CF₃)OH, CF₃, CH₂NH₂, CH₂NHC(O)Me, CH(CH₂F)NHC(O)Me, CH₂NHS(O)₂Me, C(O)NH₂, C(O)NHMe, C(O)NH—CH₂CH₂OH, C(O)NH—CH₂CH₂NH₂, C(O)NH-(pyridin-2-ylmethyl), imidazolyl, and triazolyl.
Aspect 46. The compound of any one of aspects 33 to 45, or a pharmaceutically acceptable salt thereof, wherein R¹² is selected from the group consisting of CH₂OH, CH(Me)OH, CH(CH₂F)OH, and CH₂NHC(O)Me.
Aspect 47. The compound of any one of aspects 33 to 46, or a pharmaceutically acceptable salt thereof, wherein R¹² is —CH₂OH or —CH₂NHC(O)CH₃.
Aspect 48. The compound any one of aspects 33 to 42, or a pharmaceutically acceptable salt thereof, wherein R² is C₁₋₂ alkyl substituted with —NR^{a}C(O)R^{b}, wherein each R^{a} and R^{b} is independently selected from the group consisting of hydrogen and C₁₋₃ alkyl, wherein each C₁₋₃ alkyl is optionally substituted with 1 to 3 substituents independently selected from hydroxyl and amino.
Aspect 49. The compound any one of aspects 33 to 48, or a pharmaceutically acceptable salt thereof, wherein R¹³ is C₃₋₆ alkyl optionally substituted with 1 to 2 substituents independently selected from halogen and —OH.
Aspect 50. The compound of any one of aspects 33 to 49, or a pharmaceutically acceptable salt thereof, wherein R¹³ is C₃₋₆ alkyl.
Aspect 51. The compound of any one of aspects 33 to 50, or a pharmaceutically acceptable salt thereof, wherein R¹³ is propyl, butyl or pentyl.
Aspect 52. The compound of any one of aspects 33 to 51, or a pharmaceutically acceptable salt thereof, wherein R¹³ is propyl or butyl.
Aspect 53. The compound any one of aspects 33 to 39, 42 to 46, or 49 to 51, or a pharmaceutically acceptable salt thereof, wherein:
R¹¹ is methyl or CF₃;
R¹² is —CH₂OH, —CH(Me)OH or —CH₂NHC(O)CH₃; and
R¹³ is selected from the group consisting of propyl, butyl and pentyl.
Aspect 54. The compound of any one of aspects 33, 34, or 36 to 38, wherein the moiety

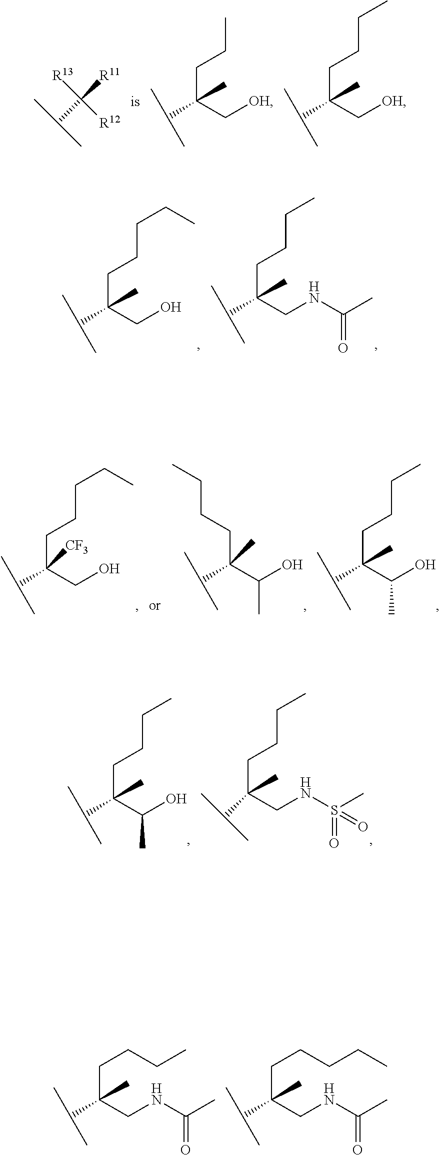

Aspect 55. The compound of any one of aspects 33 or 35 to 38, wherein the moiety

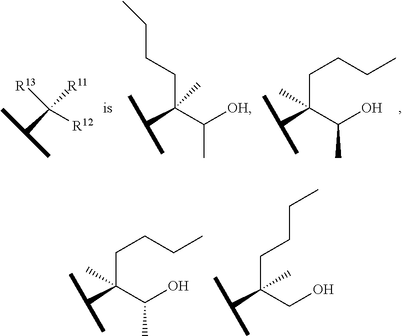

Aspect 56. The compound of any one of aspects 33, 34, 36 to 40, 42 to 47, or 49 to 53, or a pharmaceutically acceptable salt thereof, wherein the compound is a compound of Formula (IVc)

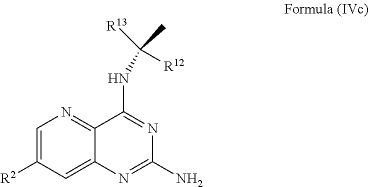

wherein
R² is hydrogen or fluoro;
R¹² is methyl substituted with 1 or 2 substituents independently selected from —OH and —NHC(O)Me; and
R¹³ is selected from the group consisting of propyl and butyl.
Aspect 57. The compound any one of aspects 33, 34, or 36, or a pharmaceutically acceptable salt thereof, wherein the compound is a compound of Formula (IVd)

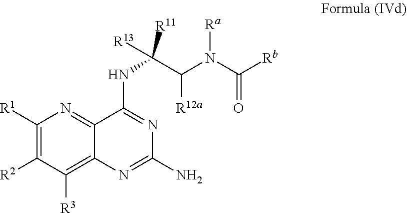

wherein
R¹ is selected from the group consisting of hydrogen, halogen, and C₁₋₃ alkyl;
R² is selected from the group consisting of hydrogen, halogen, and C₁₋₃ alkyl;
R³ is selected from the group consisting of hydrogen, halogen, and C₁₋₃ alkyl;
R¹¹ is C₁₋₂ alkyl or CF₃;
R^{12a} is selected from the group consisting of hydrogen, C₁₋₂ alkyl and C₁₋₃ haloalkyl;
R¹³ is C₃₋₆ alkyl optionally substituted with 1 to 2 halogen substituents; and
each R^{a} and R^{b} is independently selected from the group consisting of hydrogen and C₁₋₃ alkyl, wherein each C₁₋₃ alkyl is optionally substituted with 1 to 3 substituents independently selected from hydroxyl and amino.
Aspect 58. The compound of any one of aspects 33, 34 or 57, or a pharmaceutically acceptable salt thereof, wherein the compound of Formula (IVd) has the structure:

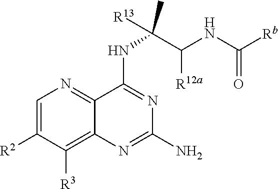

wherein
R² is selected from the group consisting of hydrogen, methyl, fluoro, and chloro; R³ is selected from the group consisting of hydrogen and methyl;
R^{12a} is selected from the group consisting of hydrogen, C₁₋₂ alkyl and C₁₋₃ haloalkyl;
R¹³ is C₃₋₆ alkyl; and
R^{b} is methyl or ethyl, each optionally substituted with hydroxyl or amino.
Aspect 59. The compound of any one of aspects 33, 34, 36, 37, 38, or 57, or a pharmaceutically acceptable salt thereof, wherein the compound of Formula (IVd) has the structure:

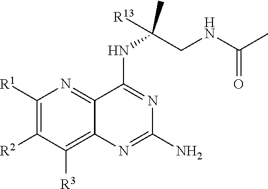

wherein
R¹³ is C₃₋₆ alkyl.
Aspect 60. The compound of any one of aspects 33, 34, 36, 37, 57 or 59, or a pharmaceutically acceptable salt thereof, wherein the compound of Formula (IVd) has the structure:

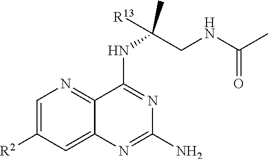

wherein
R² is selected from the group consisting of hydrogen, Cl, and F; and
R¹³ is C₃₋₆ alkyl.
Aspect 61. The compound of any one of aspects 33, 34, 57 or 59, or a pharmaceutically acceptable salt thereof, wherein the compound of Formula (IVd) has the structure:

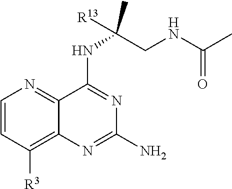

wherein
R³ is selected from the group consisting of hydrogen and methyl; and
R¹³ is C₃₋₆ alkyl.
Aspect 62. The compound of aspect 1, 2, 33, or 34, or a pharmaceutically acceptable salt thereof, having the structure:

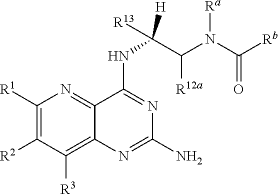

wherein
R¹ is selected from the group consisting of hydrogen, halogen, and C₁₋₃ alkyl;
R² is selected from the group consisting of hydrogen, halogen, and C₁₋₃ alkyl;
R³ is selected from the group consisting of hydrogen, halogen, and C₁₋₃ alkyl;
R^{12a} is selected from the group consisting of hydrogen, C₁₋₂ alkyl and C₁₋₃ haloalkyl;
R¹³ is C₃₋₆ alkyl optionally substituted with 1 to 2 halogen substituents; and
each R^{a} and R^{b} is independently selected from the group consisting of hydrogen and C₁₋₃ alkyl, wherein each C₁₋₃ alkyl is optionally substituted with 1 to 3 substituents independently selected from halogen, hydroxyl, amino, and C₁₋₆ haloalkyl.
Aspect 63. The compound of any one of aspects 1 to 35, or a pharmaceutically acceptable salt thereof, wherein R¹ is hydrogen, halogen, or C₁₋₆alkyl optionally substituted with 1 to 5 R²⁰ groups.
Aspect 64. The compound of any one of aspects 1 to 35 or 63, or a pharmaceutically acceptable salt thereof, wherein R¹ is hydrogen, halogen, or C₁₋₃alkyl optionally substituted with 1 to 5 halogens.
Aspect 65. The compound of any one of aspects 1 to 35, 63 or 64, or a pharmaceutically acceptable salt thereof, wherein R¹ is hydrogen, Cl, CH₃, or CF₃.
Aspect 66. The compound of any one of aspects 1 to 35 or 63 to 65, or a pharmaceutically acceptable salt thereof, wherein R² is hydrogen, halogen, —OH, CN, or C₁₋₆alkyl optionally substituted with 1 to 5 R²⁰ groups.
Aspect 67. The compound of any one of aspects 1 to 35 or 63 to 66, or a pharmaceutically acceptable salt thereof, wherein R² is hydrogen, halogen, —OH, CN or C₁₋₃alkyl optionally substituted with 1 to 5 halogens.
Aspect 68. The compound of any one aspects 1 to 35 or 63 to 67, or a pharmaceutically acceptable salt thereof, wherein R² is hydrogen, CH₃, —OH, —CF₃, —CH₂CH₃, F, Br, Cl, or CN.
Aspect 69. The compound of any one of aspects 1 to 35 or 63 to 68, or a pharmaceutically acceptable salt thereof, wherein R³ is hydrogen, halogen, or C₁₋₆alkyl optionally substituted with 1 to 5 R²⁰ groups.
Aspect 70. The compound of any one of aspects 1 to 35 or 63 to 69, or a pharmaceutically acceptable salt thereof, wherein R³ is hydrogen, halogen, or C₁₋₃alkyl optionally substituted with 1 to 5 R²⁰ groups.
Aspect 71. The compound of any one of aspects 1 to 35 or 63 to 70, or a pharmaceutically acceptable salt thereof, wherein R³ is hydrogen, Cl, or CH₃.
Aspect 72. The compound of any one of aspects 1 to 33, selected from

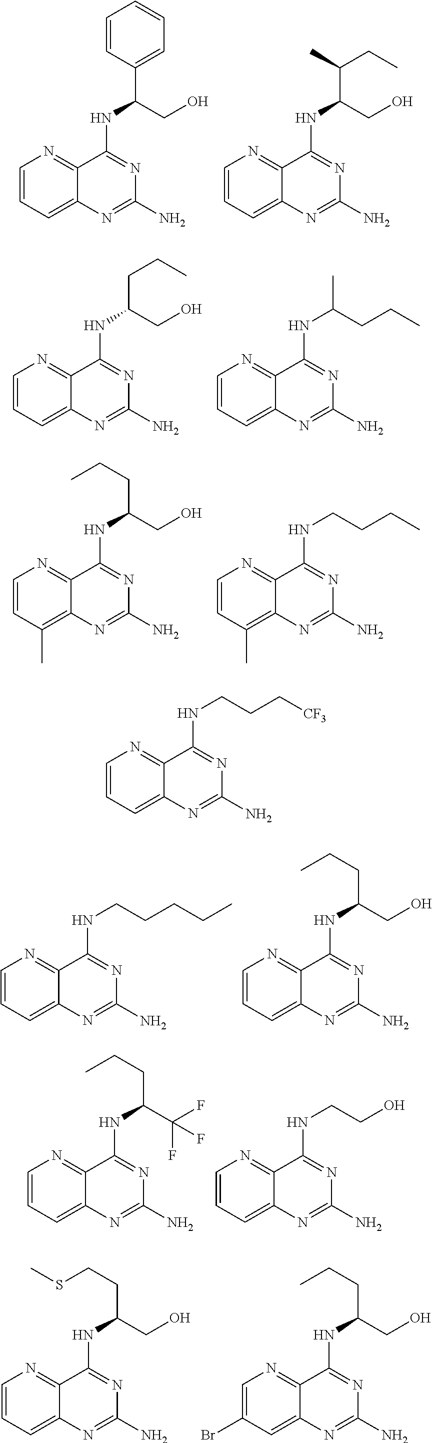

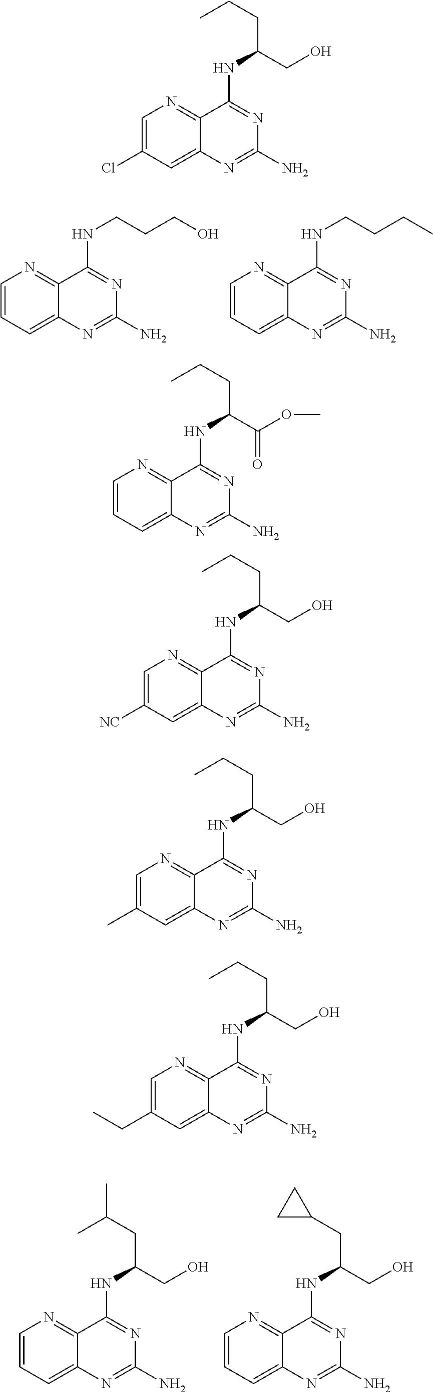

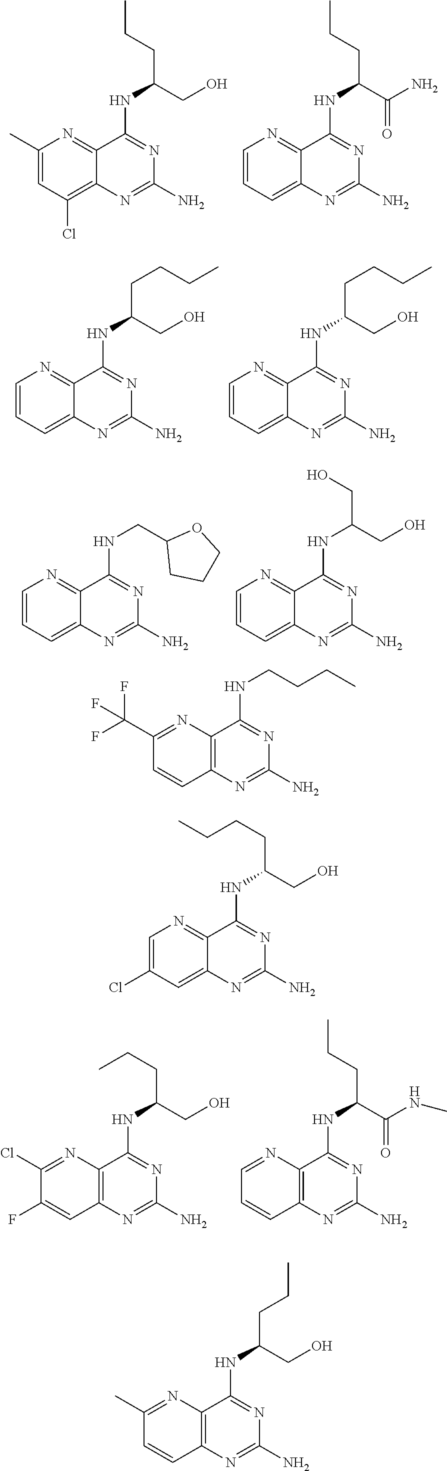

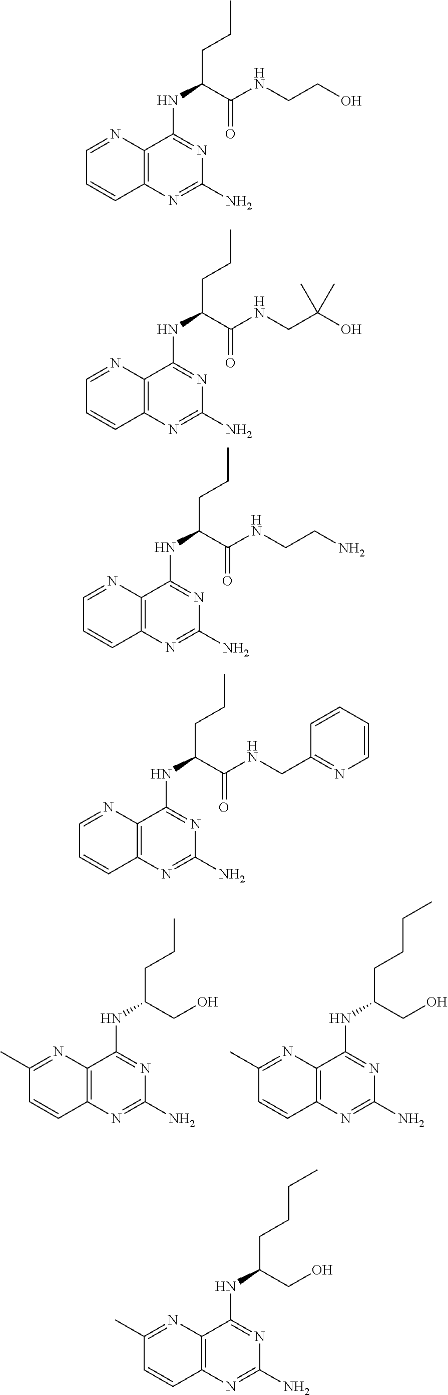

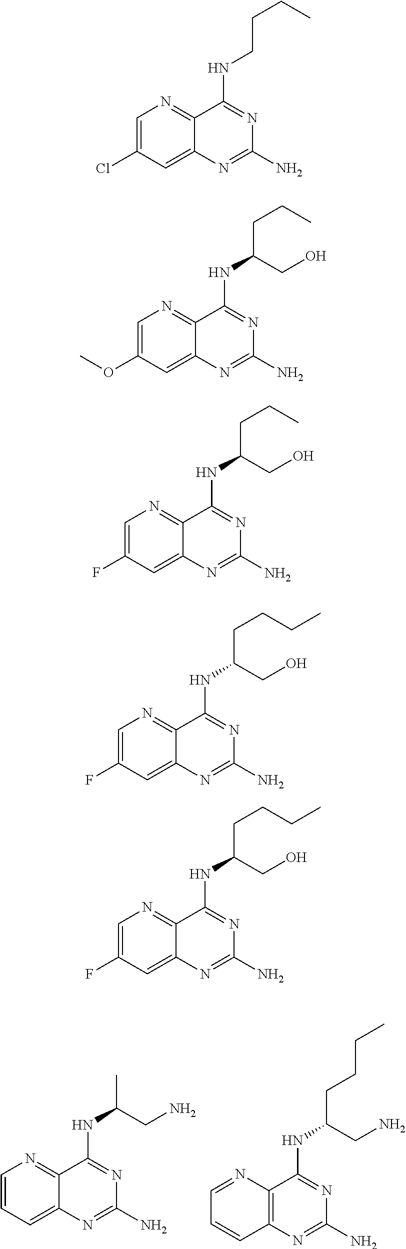

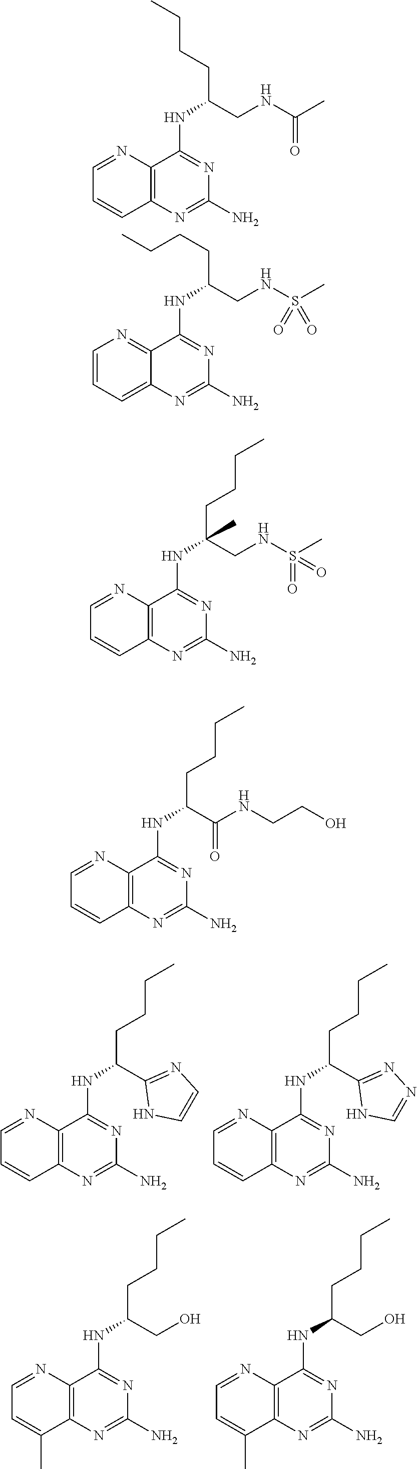

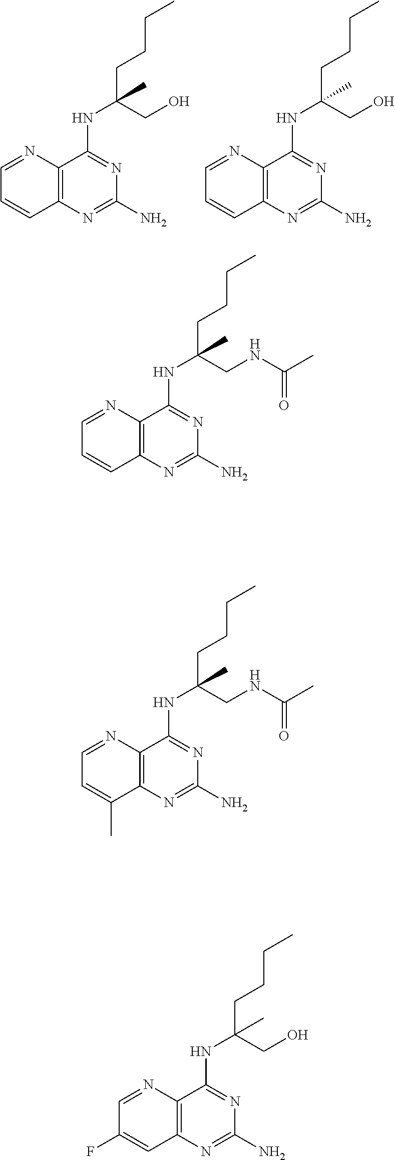

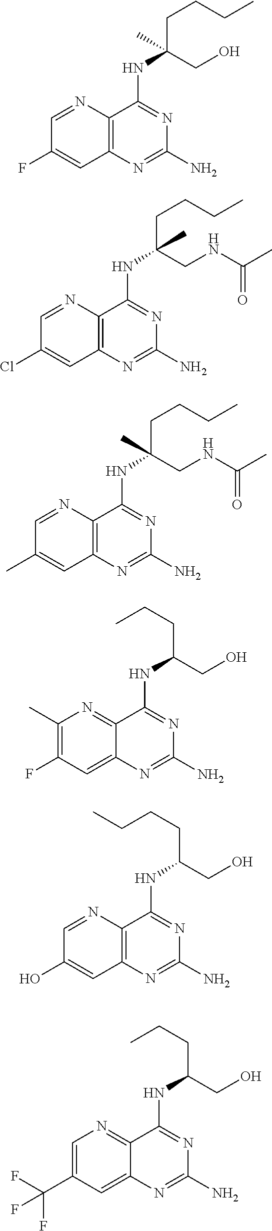

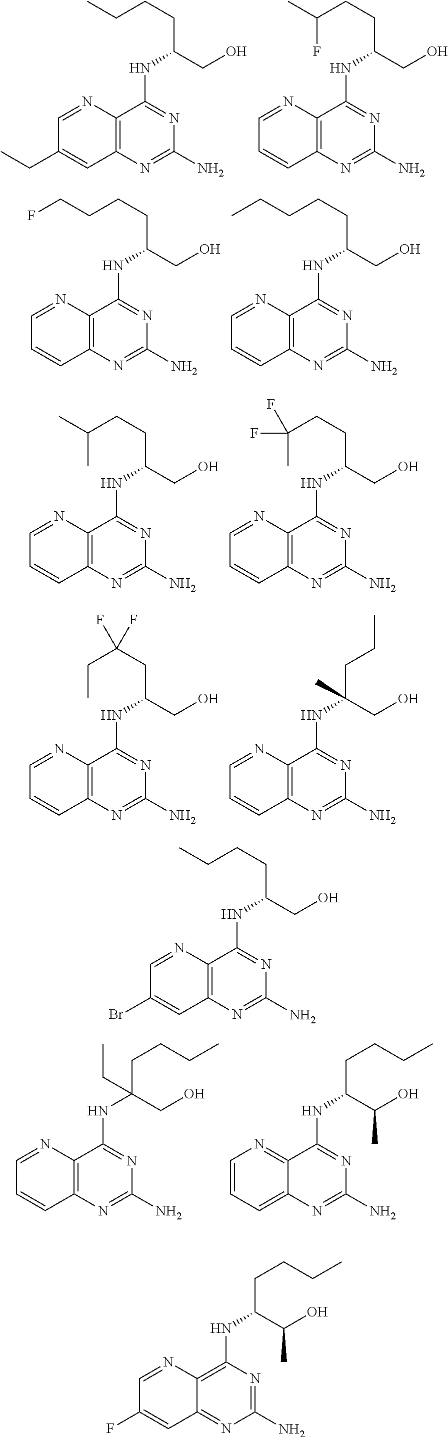

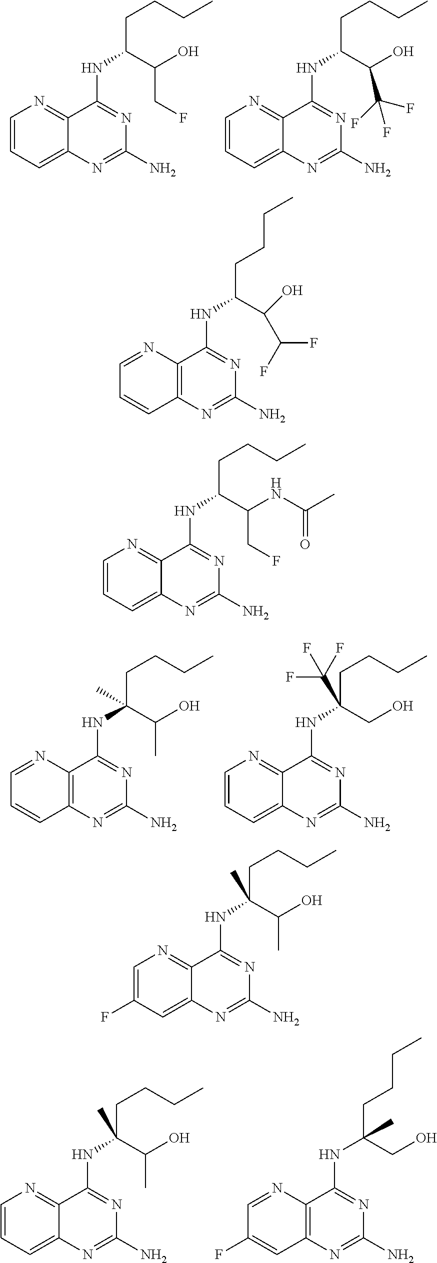

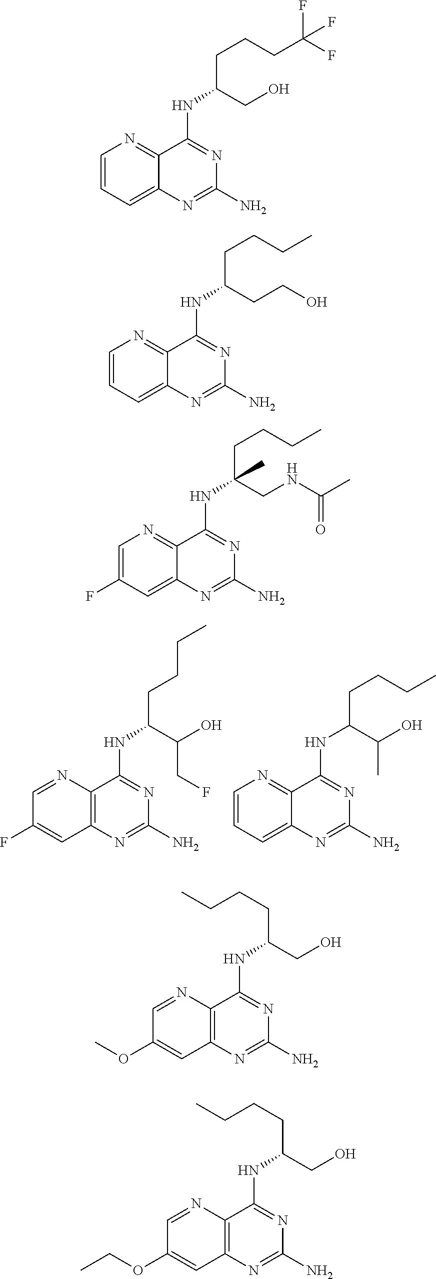

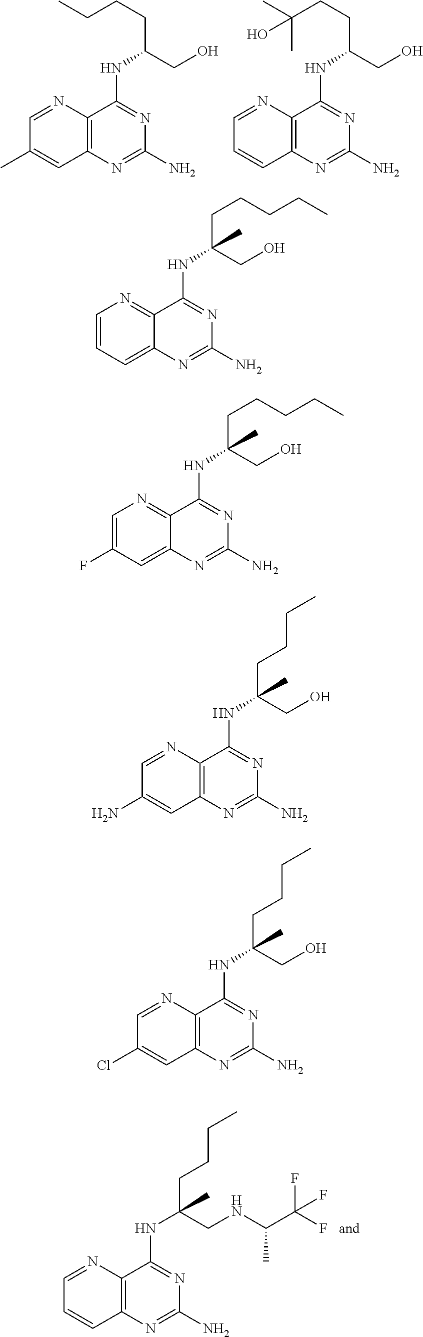

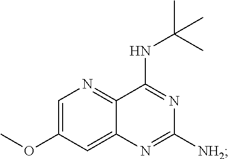 [SMILES: COc1cnc2c(NC(C)(C)C)nc(N)nc2c1]

or a pharmaceutically acceptable salt thereof.
Aspect 73. The compound of any one of aspects 1 to 34, 54, or 63 to 71, selected from

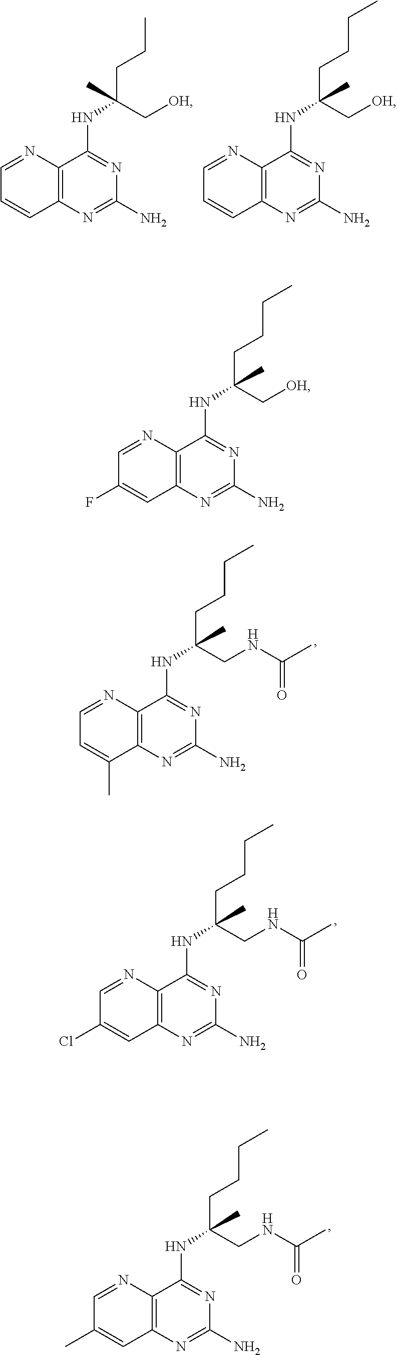

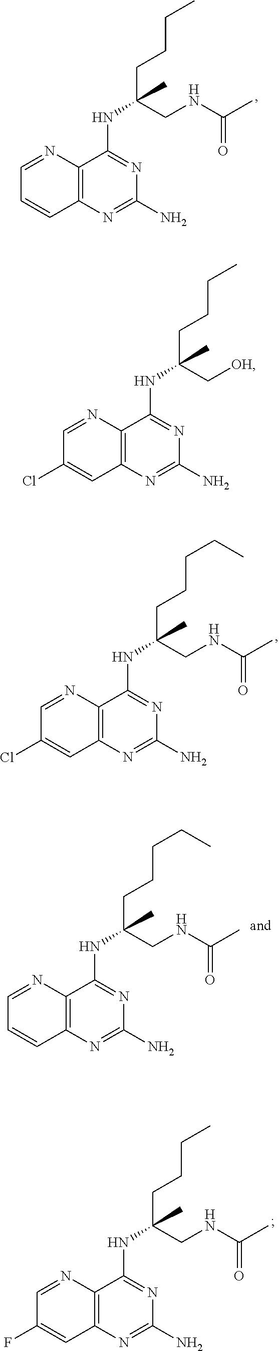

or a pharmaceutically acceptable salt thereof.
Aspect 74. A pharmaceutical composition comprising a compound of any of aspects 1 to 73, or a pharmaceutically acceptable salt thereof, and a pharmaceutically acceptable excipient.
Aspect 75. The pharmaceutical composition of aspect 74, further comprising one or more additional therapeutic agents.
Aspect 76. The pharmaceutical composition of aspect 75, wherein one or more additional therapeutic agents are selected from the group consisting of HBV DNA polymerase inhibitors, toll-like receptor 7 modulators, toll-like receptor 8 modulators, toll-like receptor 7 and 8 modulators, toll-like receptor 3 modulators, interferon alpha ligands, HBsAg inhibitors, compounds targeting HbcAg, cyclophilin inhibitors, HBV therapeutic vaccines, HBV prophylactic vaccines, HBV viral entry inhibitors, NTCP inhibitors, antisense oligonucleotide targeting viral mRNA, short interfering RNAs (siRNA), hepatitis B virus E antigen inhibitors, HBx inhibitors, cccDNA inhibitors, HBV antibodies including HBV antibodies targeting the surface antigens of the hepatitis B virus, thymosin agonists, cytokines, nucleoprotein inhibitors (HBV core or capsid protein inhibitors), stimulators of retinoic acid-inducible gene 1, stimulators of NOD2, recombinant thymosin alpha-1 and hepatitis B virus replication inhibitors, hepatitis B surface antigen (HBsAg) secretion or assembly inhibitors, IDO inhibitors, and combinations thereof.
Aspect 77. The pharmaceutical composition of aspect 75 or 76, wherein one or more additional therapeutic agents are selected from the group consisting of adefovir (Hepsera®), tenofovir disoproxil fumarate+emtricitabine (Truvada®), tenofovir disoproxil fumarate (Viread®), entecavir (Baraclude®), lamivudine (Epivir-HBV®), tenofovir alafenamide, tenofovir, tenofovir disoproxil, tenofovir alafenamide fumarate, tenofovir alafenamide hemifumarate, telbivudine (Tyzeka®), Clevudine®, emtricitabine (Emtriva®), peginterferon alfa-2b (PEG-Intron®), Multiferon®, interferon alpha 1b (Hapgen®), interferon alpha-2b (Intron A®), pegylated interferon alpha-2a (Pegasys®), interferon alfa-n1 (Humoferon®), ribavirin, interferon beta-1a (Avonex®), Bioferon, Ingaron, Inmutag (Inferon), Algeron, Roferon-A, Oligotide, Zutectra, Shaferon, interferon alfa-2b (Axxo), Alfaferone, interferon alfa-2b (BioGeneric Pharma), Feron, interferon-alpha 2 (CJ), Bevac, Laferonum, Vipeg, Blauferon-B, Blauferon-A, Intermax Alpha, Realdiron, Lanstion, Pegaferon, PDferon-B, interferon alfa-2b (IFN, Laboratorios Bioprofarma), alfainterferona 2b, Kalferon, Pegnano, Feronsure, PegiHep, interferon alfa 2b (Zydus-Cadila), Optipeg A, Realfa 2B, Reliferon, interferon alfa-2b (Amega), interferon alfa-2b (Virchow), peginterferon alfa-2b (Amega), Reaferon-EC, Proquiferon, Uniferon, Urifron, interferon alfa-2b (Changchun Institute of Biological Products), Anterferon, Shanferon, MOR-22, interleukin-2 (IL-2), recombinant human interleukin-2, Layfferon, Ka Shu Ning, Shang Sheng Lei Tai, Intefen, Sinogen, Fukangtai, Alloferon, and celmoleukin, and combinations thereof.
Aspect 78. The pharmaceutical composition of any one of aspects 75 to 77, wherein one or more additional therapeutic agents are selected from the group consisting of entecavir, adefovir, tenofovir disoproxil fumarate, tenofovir alafenamide, tenofovir, tenofovir disoproxil, tenofovir alafenamide fumarate, tenofovir alafenamide hemifumarate, telbivudine and lamivudine.
Aspect 79. The composition of aspect 75, wherein one or more additional therapeutic agents are selected from HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, pharmacokinetic enhancers, and combinations thereof.
Aspect 80. A method of modulating TLR-8, comprising administering a compound of any of aspects 1-73, or a pharmaceutically acceptable salt thereof, to a human.
Aspect 81. A method of treating or preventing a disease or condition responsive to the modulation of TLR-8, comprising administering to a human a therapeutically effective amount of a compound of any of aspects 1-73, or a pharmaceutically acceptable salt thereof.
Aspect 82. The method of aspect 80 or 81, further comprising administering one or more additional therapeutic agents.
Aspect 83. A method of treating or preventing a viral infection, comprising administering to an individual in need thereof a therapeutically effective amount of a compound of any one of aspects 1-73, or a pharmaceutically acceptable salt thereof.
Aspect 84. A method of treating or preventing a hepatitis B viral infection, comprising administering to an individual in need thereof a therapeutically effective amount of a compound of any one of aspects 1-73, or a pharmaceutically acceptable salt thereof.
Aspect 85. The method of aspect 84, further comprising administering one or more additional therapeutic agents.
Aspect 86. The method of aspect 84 or 85, comprising administering one, two, three, or four additional therapeutic agents selected from the group consisting of HBV DNA polymerase inhibitors, toll-like receptor 7 modulators, toll-like receptor 8 modulators, Toll-like receptor 7 and 8 modulators, Toll-like receptor 3 modulators, interferon alpha ligands, HBsAg inhibitors, compounds targeting HbcAg, cyclophilin inhibitors, HBV therapeutic vaccines, HBV prophylactic vaccines, HBV viral entry inhibitors, NTCP inhibitors, antisense oligonucleotide targeting viral mRNA, short interfering RNAs (siRNA), hepatitis B virus E antigen inhibitors, HBx inhibitors, cccDNA inhibitors, HBV antibodies including HBV antibodies targeting the surface antigens of the hepatitis B virus, thymosin agonists, cytokines, nucleoprotein inhibitors (HBV core or capsid protein inhibitors), stimulators of retinoic acid-inducible gene 1, stimulators of NOD2, recombinant thymosin alpha-1 and hepatitis B virus replication inhibitors, hepatitis B surface antigen (HBsAg) secretion or assembly inhibitors, IDO inhibitors, and combinations thereof.
Aspect 87. The method of any one of aspects 84 to 86, comprising administering one, two, three, or four additional therapeutic agents selected from the group consisting of adefovir (Hepsera®), tenofovir disoproxil fumarate+emtricitabine (Truvada®), tenofovir disoproxil fumarate (Viread®), entecavir (Baraclude®), lamivudine (Epivir-HBV®), tenofovir alafenamide, tenofovir, tenofovir disoproxil, tenofovir alafenamide fumarate, tenofovir alafenamide hemifumarate, telbivudine (Tyzeka®), Clevudine®, emtricitabine (Emtriva®), peginterferon alfa-2b (PEG-Intron®), Multiferon®, interferon alpha 1b (Hapgen®), interferon alpha-2b (Intron A®), pegylated interferon alpha-2a (Pegasys®), interferon alfa-n1 (Humoferon®), ribavirin, interferon beta-1a (Avonex®), Bioferon, Ingaron, Inmutag (Inferon), Algeron, Roferon-A, Oligotide, Zutectra, Shaferon, interferon alfa-2b (Axxo), Alfaferone, interferon alfa-2b, Feron, interferon-alpha 2 (CJ), Bevac, Laferonum, Vipeg, Blauferon-B, Blauferon-A, Intermax Alpha, Realdiron, Lanstion, Pegaferon, PDferon-B, alfainterferona 2b, Kalferon, Pegnano, Feronsure, PegiHep, Optipeg A, Realfa 2B, Reliferon, peginterferon alfa-2b, Reaferon-EC, Proquiferon, Uniferon, Urifron, interferon alfa-2b, Anterferon, Shanferon, MOR-22, interleukin-2 (IL-2), recombinant human interleukin-2 (Shenzhen Neptunus), Layfferon, Ka Shu Ning, Shang Sheng Lei Tai, Intefen, Sinogen, Fukangtai, Alloferon and celmoleukin.
Aspect 88. The method of any one of aspects 84 to 86, comprising administering one, two, three, or four additional therapeutic agents selected from entecavir, adefovir, tenofovir disoproxil fumarate, tenofovir alafenamide, tenofovir, tenofovir disoproxil, tenofovir alafenamide fumarate, tenofovir alafenamide hemifumarate, telbivudine and lamivudine.
Aspect 89. A method of treating or preventing a HIV infection, comprising administering to an individual in need thereof a therapeutically effective amount of a compound of any one of aspects 1-73, or a pharmaceutically acceptable salt thereof.
Aspect 90. The method of aspect 89, comprising administering one or more additional therapeutic agents.
Aspect 91. The method of aspect 89 or 90, comprising administering one, two, three, or four additional therapeutic agents selected from the group consisting of HIV protease inhibiting compounds, HIV non-nucleoside inhibitors of reverse transcriptase, HIV nucleoside inhibitors of reverse transcriptase, HIV nucleotide inhibitors of reverse transcriptase, HIV integrase inhibitors, gp41 inhibitors, CXCR4 inhibitors, gp120 inhibitors, CCR5 inhibitors, capsid polymerization inhibitors, and other drugs for treating or preventing HIV, and combinations thereof.
Aspect 92. The method of any one of aspects 89 to 91, comprising administering one, two, three, or four additional therapeutic agents selected from Triumeq® (dolutegravir+abacavir+lamivudine), dolutegravir+abacavir sulfate+lamivudine, raltegravir, Truvada® (tenofovir disoproxil fumarate+emtricitabine, TDF+FTC), maraviroc, enfuvirtide, Epzicom® (Livexa®, abacavir sulfate+lamivudine, ABC+3TC), Trizivir® (abacavir sulfate+zidovudine+lamivudine, ABC+AZT+3TC), adefovir, adefovir dipivoxil, Stribild® (elvitegravir+cobicistat+tenofovir disoproxil fumarate+emtricitabine), rilpivirine, rilpivirine hydrochloride, Complera® (Eviplera®, rilpivirine+tenofovir disoproxil fumarate+emtricitabine), cobicistat, Atripla® (efavirenz+tenofovir disoproxil fumarate+emtricitabine), atazanavir, atazanavir sulfate, dolutegravir, elvitegravir, Aluvia® (Kaletra®, lopinavir+ritonavir), ritonavir, emtricitabine, atazanavir sulfate+ritonavir, darunavir, lamivudine, Prolastin, fosamprenavir, fosamprenavir calcium, efavirenz, Combivir® (zidovudine+lamivudine, AZT+3TC), etravirine, nelfinavir, nelfinavir mesylate, interferon, didanosine, stavudine, indinavir, indinavir sulfate, tenofovir+lamivudine, zidovudine, nevirapine, saquinavir, saquinavir mesylate, aldesleukin, zalcitabine, tipranavir, amprenavir, delavirdine, delavirdine mesylate, Radha-108 (Receptol), Hlviral, lamivudine+tenofovir disoproxil fumarate, efavirenz+lamivudine+tenofovir disoproxil fumarate, phosphazid, lamivudine+nevirapine+zidovudine, (2R,5S,13aR)—N-(2,4-difluorobenzyl)-8-hydroxy-7,9-dioxo-2,3,4,5,7,9,13,13a-octahydro-2,5-methanopyrido[1′,2′:4,5]pyrazino[2,1-b][1,3]oxazepine-10-carboxamide, (2S,5R,13aS)—N-(2,4-difluorobenzyl)-8-hydroxy-7,9-dioxo-2,3,4,5,7,9,13,13a-octahydro-2,5-methanopyrido[1′,2′:4,5]pyrazino[2,1-b][1,3]oxazepine-10-carboxamide, (1S,4R,12aR)—N-(2,4-difluorobenzyl)-7-hydroxy-6,8-dioxo-1,2,3,4,6,8,12,12a-octahydro-1,4-methanodipyrido[1,2-a:1′,2′-d]pyrazine-9-carboxamide, (1R,4S,12aR)-7-hydroxy-6,8-dioxo-N-(2,4,6-trifluorobenzyl)-1,2,3,4,6,8,12,12a-octahydro-1,4-methanodipyrido[1,2-a:1′,2′-d]pyrazine-9-carboxamide, (2R,5S,13aR)-8-hydroxy-7,9-dioxo-N-(2,4,6-trifluorobenzyl)-2,3,4,5,7,9,13,13a-octahydro-2,5-methanopyrido[1′,2′:4,5]pyrazino[2,1-b][1,3]oxazepine-10-carboxamide, and (1R,4S,12aR)—N-(2,4-difluorobenzyl)-7-hydroxy-6,8-dioxo-1,2,3,4,6,8,12,12a-octahydro-1,4-methanodipyrido[1,2-a:1′,2′-d]pyrazine-9-carboxamide, abacavir, abacavir sulfate, tenofovir, tenofovir disoproxil, tenofovir disoproxil fumarate, tenofovir alafenamide and tenofovir alafenamide hemifumarate.
Aspect 93. A method of treating a hyperproliferative disease, comprising administering to an individual in need thereof a therapeutically effective amount of a compound of any one of aspects 1-73, or a pharmaceutically acceptable salt thereof.
Aspect 94. The method of aspect 93, further comprising administering one or more additional therapeutic agents.
Aspect 95. The method of aspect 93 or 94, wherein the hyperproliferative disease is cancer.
Aspect 96. The method of aspect 95, wherein the cancer is prostate cancer, breast cancer, ovarian cancer, hepatocellular carcinoma, gastric cancer, colorectal cancer or recurrent or metastatic squamous cell carcinoma.
Aspect 97. A kit comprising a compound of any of aspects 1-73, or a pharmaceutically acceptable salt thereof.
Aspect 98. An article of manufacture comprising a unit dosage of a compound of any of aspects 1-73.
Aspect 99. A compound of any of aspects 1-73, or a pharmaceutically acceptable salt thereof for use in medical therapy.
Aspect 100. A compound of any of aspects 1-73, or a pharmaceutically acceptable salt thereof, for use in treating or preventing a HBV infection in a human.
Aspect 101. The use of a compound of any of aspects 1-73, or a pharmaceutically acceptable salt thereof, for the manufacture of a medicament for use in medical therapy.
Aspect 102. A compound of any of aspects 1-73 or a pharmaceutically acceptable salt thereof, for use in modulating a toll-like receptor in vitro.

### EXAMPLES

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

### Example 1. HBV Core Plasmid & HBV Pol Plasmid

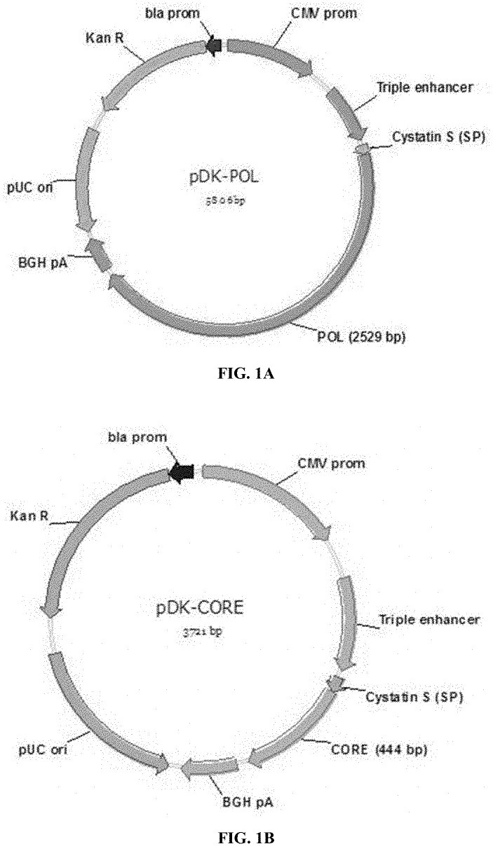

A schematic representation of the pDK-pol and pDK-core vectors is shown in FIGS. 1A and 1B, respectively. An HBV core or pol antigen optimized expression cassette containing a CMV promoter (SEQ ID NO: 18), a splicing enhancer (triple composite sequence) (SEQ ID NO: 10), Cystatin S precursor signal peptide SPCS (NP_0018901.1) (SEQ ID NO: 9), and pol (SEQ ID NO: 5) or core (SEQ ID NO: 2) gene was introduced into a pDK plasmid backbone, using standard molecular biology techniques.

The plasmids were tested in vitro for core and pol antigen expression by Western blot analysis using core and pol specific antibodies, and were shown to provide consistent expression profile for cellular and secreted core and pol antigens (data not shown).

### Example 2. Generation of Adenoviral Vectors Expressing a Fusion of Truncated HBV Core Antigen with HBV Pol Antigen

The creation of an adenovirus vector has been designed as a fusion protein expressed from a single open reading frame. Additional configurations for the expression of the two proteins, e.g. using two separate expression cassettes, or using a 2A-like sequence to separate the two sequences, can also be envisaged.

### Design of Expression Cassettes for Adenoviral Vectors

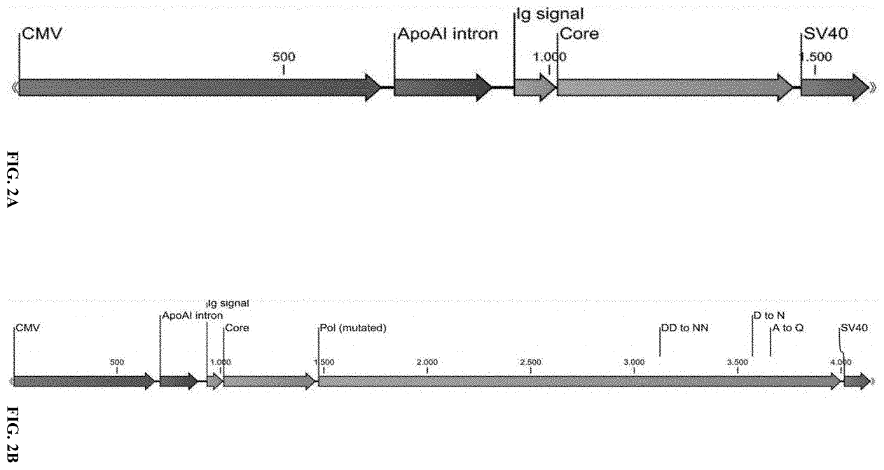

The expression cassettes (diagrammed in FIG. 2A and FIG. 2B) are comprised of the CMV promoter (SEQ ID NO: 19), an intron (SEQ ID NO:12) (a fragment derived from the human ApoAI gene—GenBank accession X01038 base pairs 295-523, harboring the ApoAI second intron), followed by the optimized coding sequence—either core alone or the core and polymerase fusion protein preceded by a human immunoglobulin secretion signal coding sequence (SEQ ID NO: 14), and followed by the SV40 polyadenylation signal (SEQ ID NO: 13). A secretion signal was included because of past experience showing improvement in the manufacturability of some adenoviral vectors harboring secreted transgenes, without influencing the elicited T-cell response (mouse experiments).
The last two residues of the Core protein (VV) and the first two residues of the Polymerase protein (MP) if fused results in a junction sequence (VVMP) that is present on the human dopamine receptor protein (D3 isoform), along with flanking homologies.
The interjection of an AGAG linker between the core and the polymerase sequences eliminates this homology and returned no further hits in a Blast of the human proteome.

### Example 3. In Vivo Immunogenicity Study of DNA Vaccine in Mice

An immunotherapeutic DNA vaccine containing DNA plasmids encoding an HBV core antigen or HBV polymerase antigen was tested in mice. The purpose of the study was designed to detect T-cell responses induced by the vaccine after intramuscular delivery via electroporation into BALB/c mice. Initial immunogenicity studies focused on determining the cellular immune responses that would be elicited by the introduced HBV antigens.
In particular, the plasmids tested included a pDK-Pol plasmid and pDK-Core plasmid, as shown in FIGS. 1A and 1B, respectively, and as described above in Example 1. The pDK-Pol plasmid encoded a polymerase antigen having the amino acid sequence of SEQ ID NO: 7, and the pDK-Core plasmid encoding a Core antigen having the amino acid sequence of SEQ ID NO: 2. First, T-cell responses induced by each plasmid individually were tested. The DNA plasmid (pDNA) vaccine was intramuscularly delivered via electroporation to Balb/c mice using a commercially available TriGrid™ delivery system-intramuscular (TDS-IM) adapted for application in the mouse model in cranialis tibialis. See International Patent Application Publication WO2017172838, and U.S. Patent Application No. 62/607,430, entitled “Method and Apparatus for the Delivery of Hepatitis B Virus (HBV) Vaccines,” filed on Dec. 19, 2017 for additional description on methods and devices for intramuscular delivery of DNA to mice by electroporation, the disclosures of which are hereby incorporated by reference in their entireties. In particular, the TDS-IM array of a TDS-IM v1.0 device having an electrode array with a 2.5 mm spacing between the electrodes and an electrode diameter of 0.030 inch was inserted percutaneously into the selected muscle, with a conductive length of 3.2 mm and an effective penetration depth of 3.2 mm, and with the major axis of the diamond configuration of the electrodes oriented in parallel with the muscle fibers. Following electrode insertion, the injection was initiated to distribute DNA (e.g., 0.020 ml) in the muscle. Following completion of the IM injection, a 250 V/cm electrical field (applied voltage of 59.4-65.6 V, applied current limits of less than 4 A, 0.16 A/sec) was locally applied for a total duration of about 400 ms at a 10% duty cycle (i.e., voltage is actively applied for a total of about 40 ms of the about 400 ms duration) with 6 total pulses. Once the electroporation procedure was completed, the TriGrid™ array was removed and the animals were recovered. High-dose (20 μg) administration to BALB/c mice was performed as summarized in Table 1. Six mice were administered plasmid DNA encoding the HBV core antigen (pDK-core; Group 1), six mice were administered plasmid DNA encoding the HBV pol antigen (pDK-pol; Group 2), and two mice received empty vector as the negative control. Animals received two DNA immunizations two weeks apart and splenocytes were collected one week after the last immunization.

TABLE 1
Mouse immunization experimental design of the pilot study.

| | | | Unilateral | | | | Endpoint |
|---|---|---|---|---|---|---|---|
| | | | Admin Site | | | | (spleen |
| | | | (alternate | | | Admin | harvest) |
| Group | N | pDNA | sides) | Dose | Vol | Days | Day |
| 1 | 6 | Core | CT + EP | 20 μg | 20 μL | 0, 14 | 21 |
| 2 | 6 | Pol | CT + EP | 20 μg | 20 μL | 0, 14 | 21 |
| 3 | 2 | Empty | CT + EP | 20 μg | 20 μL | 0, 14 | 21 |
| | | Vector | | | | | |
| | | (neg | | | | | |
| | | control) | | | | | |

CT, cranialis tibialis muscle;
EP, electroporation.

Antigen-specific responses were analyzed and quantified by IFN-γ enzyme-linked immunospot (ELISPOT). In this assay, isolated splenocytes of immunized animals were incubated overnight with peptide pools covering the Core protein, the Pol protein, or the small peptide leader and junction sequence (2 μg/ml of each peptide). These pools consisted of 15 mer peptides that overlap by 11 residues matching the Genotypes BCD consensus sequence of the Core and Pol vaccine vectors. The large 94 kDan HBV Pol protein was split in the middle into two peptide pools. Antigen-specific T cells were stimulated with the homologous peptide pools and IFN-γ-positive T cells were assessed using the ELISPOT assay. IFN-γ release by a single antigen-specific T cell was visualized by appropriate antibodies and subsequent chromogenic detection as a colored spot on the microplate referred to as spot-forming cell (SFC).

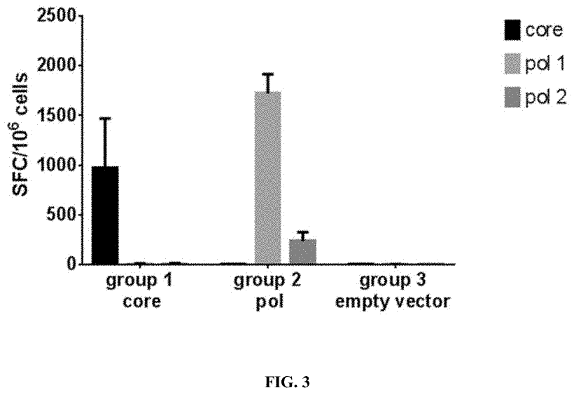

Substantial T-cell responses against HBV Core were achieved in mice immunized with the DNA vaccine plasmid pDK-Core (Group 1) reaching 1,000 SFCs per 10⁶ cells (FIG. 3). Pol T-cell responses towards the Pol 1 peptide pool were strong (˜1,000 SFCs per 10⁶ cells). The weak Pol-2-directed anti-Pol cellular responses were likely due to the limited MHC diversity in mice, a phenomenon called T-cell immunodominance defined as unequal recognition of different epitopes from one antigen. A confirmatory study was performed confirming the results obtained in this study (data not shown).

The above results demonstrate that vaccination with a DNA plasmid vaccine encoding HBV antigens induces cellular immune responses against the administered HBV antigens in mice. Similar results were also obtained with non-human primates (data not shown).

It is understood that the examples and embodiments described herein are for illustrative purposes only, and that changes could be made to the embodiments described above without departing from the broad inventive concept thereof. It is understood, therefore, that this invention is not limited to the particular embodiments disclosed, but it is intended to cover modifications within the spirit and scope of the invention as defined by the appended claims.

1. A therapeutic combination for use in treating a hepatitis B virus (HBV) infection in a subject in need thereof, comprising:
i) at least one of:
a) a truncated HBV core antigen consisting of an amino acid sequence that is at least 95% identical to SEQ ID NO: 2,
b) a first non-naturally occurring nucleic acid molecule comprising a first polynucleotide sequence encoding the truncated HBV core antigen,
c) an HBV polymerase antigen having an amino acid sequence that is at least 90% identical to SEQ ID NO: 7, wherein the HBV polymerase antigen does not have reverse transcriptase activity and RNase H activity, and
d) a second non-naturally occurring nucleic acid molecule comprising a second polynucleotide sequence encoding the HBV polymerase antigen; and
ii) a compound selected from:
1) a benzazepine carboxamide compound of formula (K) 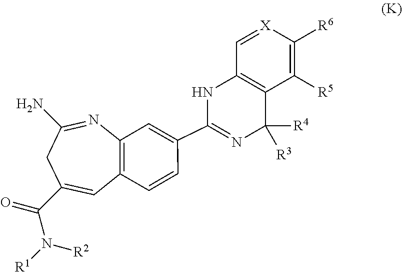
or a pharmaceutically acceptable salt thereof,
wherein R¹ is C₃₋₇-alkyl,
wherein R² is C₃₋₇-alkyl or C₃₋₇-cycloalkyl-C₁₋₇-alkyl,
wherein R³ is hydrogen or C₁₋₇-alkyl,
wherein R⁴ is hydrogen or C₁₋₇-alkyl,
wherein R⁵ is selected from the group consisting of hydrogen, halogen, C₁₋₇-alkyl and C₁₋₇-alkoxy,
wherein R⁶ is selected from the group consisting of hydrogen, halogen, C₁₋₇-alkyl and C₁₋₇-alkoxy,
wherein X is N or CR⁷, and
wherein R⁷ is selected from the group consisting of hydrogen, halogen, C₁₋₇-alkyl and C₁₋₇-alkoxy;
2) a pyridopyrimidine compound of formula (J) 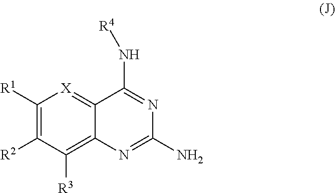
or a pharmaceutically acceptable salt thereof,
wherein X is N or CR¹⁰,
wherein R¹ is selected from the group consisting of hydrogen, halogen, C₁₋₆alkyl, CN, —NR^{a}R^{b}, —S(O)₁₋₂R^{a}, and OR^{a}, wherein the C₁₋₆alkyl is optionally substituted with 1 to 5 R²⁰ groups,
wherein R² is selected from the group consisting of hydrogen, halogen, C₁₋₆alkyl, CN, —NR^{a}R^{b}, —S(O)₁₋₂R^{a} and OR^{a}, wherein the C₁₋₆alkyl is optionally substituted with 1 to 5 R²⁰ groups,
wherein R³ is selected from the group consisting of hydrogen, halogen, C₁₋₆alkyl, CN, —NR^{a}R^{b}, —S(O)₁₋₂R^{a}, and OR^{a}, wherein the C₁₋₆alkyl is optionally substituted with 1 to 5 R²⁰ groups,
wherein R⁴ is C₁₋₁₂ alkyl which is optionally substituted with 1 to 5 substituents independently selected from halogen, —OR^{a}, —NR^{a}R^{b},
CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆haloalkyl, C₃₋₆cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur,
wherein each of the C₃₋₆cycloalkyl, 3 to 6 membered heterocyclyl, C₆₋₁₀ aryl, and 5 to 10 membered heteroaryl is optionally substituted with 1 to 5 R²¹ groups,
wherein R¹⁰ is selected from hydrogen, halogen, C₁₋₆alkyl, CN, —NR^{a}R^{b}, —S(O)₁₋₂R^{a}, and OR^{a}, wherein the C₁₋₆alkyl is optionally substituted with 1 to 5 R²⁰ groups,
wherein each R²⁰ is independently selected from the group consisting of halogen, C₁₋₆haloalkyl, CN, —NR^{a}R^{b}, S(O)₁₋₂R^{a}, and OR^{a},
wherein each R²¹ is independently selected from the group consisting of halogen, C₁₋₆alkyl, C₁₋₆haloalkyl, CN, —NR^{a}R^{b}, S(O)₁₋₂R^{a}, and OR^{a},
wherein each R^{a} and R^{b} are independently selected from the group consisting of hydrogen and C₁₋₆alkyl, and
wherein each of the C₁₋₆alkyl is optionally substituted with 1 to 5 substituents independently selected from halogen, hydroxyl, amino, 5 to 10 membered heteroaryl, wherein the 5 to 10 membered heteroaryl has 1 to 3 heteroatoms selected from oxygen, nitrogen, and sulfur, and C₁₋₆haloalkyl,
provided that when X is N, R¹ is Cl, R² is H and R³ is H then R⁴ is not CH₂CH₂OMe or CH₂CH₂SO₂Me; and
3) a pyridopyrimidine compound of formula (I) 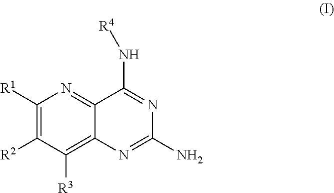
or a pharmaceutically acceptable salt thereof,
wherein R¹ is selected from the group consisting of hydrogen, halogen, C₁₋₆alkyl, CN, —NR^{a}R^{b}, —S(O)₁₋₂R^{a}, and OR^{a}, wherein the C₁₋₆alkyl is optionally substituted with 1 to 5 R²⁰ groups,
wherein R² is selected from the group consisting of hydrogen, halogen, C₁₋₆alkyl, CN, —NR^{a}R^{b}, —S(O)₁₋₂R^{a} and OR^{a}, wherein the C₁₋₆alkyl optionally substituted with 1 to 5 R²⁰ groups,
wherein R³ is selected from the group consisting of hydrogen, halogen, C₁₋₆alkyl, CN, —NR^{a}R^{b}, —S(O)₁₋₂R^{a}, and OR^{a}, wherein the C₁₋₆alkyl is optionally substituted with 1 to 5 R²⁰ groups,
wherein R⁴ is C₁₋₁₂ alkyl which is optionally substituted with 1 to 5 substituents independently selected from halogen, —OR^{a}, —NR^{a}R^{b},
CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆haloalkyl, C₃₋₆cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur,
wherein each of the C₃₋₆cycloalkyl, 3 to 6 membered heterocyclyl, C₆₋₁₀ aryl, and 5 to 10 membered heteroaryl is optionally substituted with 1 to 5 R²¹ groups,
wherein each R²⁰ is independently selected from the group consisting of halogen, C₁₋₆haloalkyl, CN, —NR^{a}R^{b}, S(O)₁₋₂R^{a}, and OR^{a},
wherein each R²¹ is independently selected from the group consisting of halogen, C₁₋₆alkyl, C₁₋₆haloalkyl, CN, —NR^{a}R^{b}, S(O)₁₋₂R^{a}, and OR^{a}, and
wherein each R^{a} and R^{b} are independently selected from the group consisting of hydrogen and C₁₋₆alkyl, wherein each of the C₁₋₆alkyl is optionally substituted with 1 to 5 substituents independently selected from halogen, hydroxyl, amino, 5 to 10 membered heteroaryl wherein the 5 to 10 membered heteroaryl has 1 to 3 heteroatoms selected from oxygen, nitrogen, and sulfur, and C₁₋₆haloalkyl,
provided that when R¹ is Cl, R² is H and R³ is H then R⁴ is not CH₂CH₂OMe or CH₂CH₂SO₂Me.

2.-3. (canceled)

4. The therapeutic combination of claim 1, comprising at least one of the HBV polymerase antigen and the truncated HBV core antigen.

5. The therapeutic combination of claim 4, comprising the HBV polymerase antigen and the truncated HBV core antigen.

6. The therapeutic combination of claim 1, comprising at least one of the first non-naturally occurring nucleic acid molecule comprising the first polynucleotide sequence encoding the truncated HBV core antigen and the second non-naturally occurring nucleic acid molecule comprising the second polynucleotide sequence encoding the HBV polymerase antigen.

7. A therapeutic combination for use in treating a hepatitis B virus (HBV) infection in a subject in need thereof, comprising:
i) a first non-naturally occurring nucleic acid molecule comprising a first polynucleotide sequence encoding a truncated HBV core antigen consisting of an amino acid sequence that is at least 95% identical to SEQ ID NO: 2; and
ii) a second non-naturally occurring nucleic acid molecule comprising a second polynucleotide sequence encoding an HBV polymerase antigen having an amino acid sequence that is at least 90% identical to SEQ ID NO: 7, wherein the HBV polymerase antigen does not have reverse transcriptase activity and RNase H activity; and
iii) a compound selected from:
1) a benzazepine carboxamide compound of formula (K) 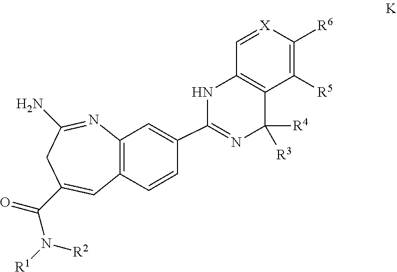
or a pharmaceutically acceptable salt thereof,
wherein R¹ is C₃₋₇-alkyl,
wherein R² is C₃₋₇-alkyl or C₃₋₇-cycloalkyl-C₁₋₇-alkyl,
wherein R³ is hydrogen or C₁₋₇-alkyl,
wherein R⁴ is hydrogen or C₁₋₇-alkyl,
wherein R⁵ is selected from the group consisting of hydrogen, halogen, C₁₋₇-alkyl and C₁₋₇-alkoxy,
wherein R⁶ is selected from the group consisting of hydrogen, halogen, C₁₋₇-alkyl and C₁₋₇-alkoxy,
wherein X is N or CR⁷, and
wherein R⁷ is selected from the group consisting of hydrogen, halogen, C₁₋₇-alkyl and C₁₋₇-alkoxy;
2) a pyridopyrimidine compound of formula (J) 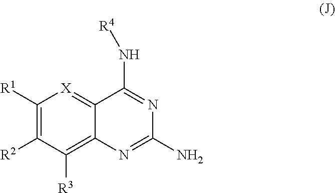
or a pharmaceutically acceptable salt thereof,
wherein X is N or CR¹⁰,
wherein R¹ is selected from the group consisting of hydrogen, halogen, C₁₋₆alkyl, CN, —NR^{a}R^{b}, —S(O)₁₋₂R^{a}, and OR^{a}, wherein the C₁₋₆alkyl is optionally substituted with 1 to 5 R²⁰ groups,
wherein R² is selected from the group consisting of hydrogen, halogen, C₁₋₆alkyl, CN, —NR^{a}R^{b}, —S(O)₁₋₂R^{a} and OR^{a}, wherein the C₁₋₆alkyl is optionally substituted with 1 to 5 R²⁰ groups,
wherein R³ is selected from the group consisting of hydrogen, halogen, C₁₋₆alkyl, CN, —NR^{a}R^{b}, —S(O)₁₋₂R^{a}, and OR^{a}, wherein the C₁₋₆alkyl is optionally substituted with 1 to 5 R²⁰ groups,
wherein R⁴ is C₁₋₁₂ alkyl which is optionally substituted with 1 to 5 substituents independently selected from halogen, —OR^{a}, —NR^{a}R^{b},
CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)₂R^{b}, C₁₋₆haloalkyl, C₃₋₆cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur,
wherein each of the C₃₋₆cycloalkyl, 3 to 6 membered heterocyclyl, C₆₋₁₀ aryl, and 5 to 10 membered heteroaryl is optionally substituted with 1 to 5 R²¹ groups,
wherein R¹⁰ is selected from hydrogen, halogen, C₁₋₆alkyl, CN, —NR^{a}R^{b}, —S(O)₁₋₂R^{a}, and OR^{a}, wherein the C₁₋₆alkyl is optionally substituted with 1 to 5 R²⁰ groups,
wherein each R²⁰ is independently selected from the group consisting of halogen, C₁₋₆haloalkyl, CN, —NR^{a}R^{b}, S(O)₁₋₂R^{a}, and OR^{a},
wherein each R²¹ is independently selected from the group consisting of halogen, C₁₋₆alkyl, C₁₋₆haloalkyl, CN, —NR^{a}R^{b}, S(O)₁₋₂R^{a}, and OR^{a},
wherein each R^{a} and R^{b} are independently selected from the group consisting of hydrogen and C₁₋₆alkyl, and
wherein each of the C₁₋₆alkyl is optionally substituted with 1 to 5 substituents independently selected from halogen, hydroxyl, amino, 5 to 10 membered heteroaryl, wherein the 5 to 10 membered heteroaryl has 1 to 3 heteroatoms selected from oxygen, nitrogen, and sulfur, and C₁₋₆haloalkyl,
provided that when X is N, R¹ is Cl, R² is H and R³ is H then R⁴ is not CH₂CH₂OMe or CH₂CH₂SO₂Me; and
3) a pyridopyrimidine compound of formula (I) 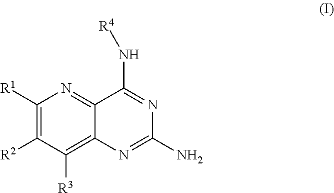
or a pharmaceutically acceptable salt thereof,
wherein R¹ is selected from the group consisting of hydrogen, halogen, C₁₋₆alkyl, CN, —NR^{a}R^{b}, —S(O)₁₋₂R^{a}, and OR^{a}, wherein the C₁₋₆alkyl is optionally substituted with 1 to 5 R²⁰ groups,
wherein R² is selected from the group consisting of hydrogen, halogen, C₁₋₆alkyl, CN, —NR^{a}R^{b}, —S(O)₁₋₂R^{a} and OR^{a}, wherein the C₁₋₆alkyl optionally substituted with 1 to 5 R²⁰ groups,
wherein R³ is selected from the group consisting of hydrogen, halogen, C₁₋₆alkyl, CN, —NR^{a}R^{b}, —S(O)₁₋₂R^{a}, and OR^{a}, wherein the C₁₋₆alkyl is optionally substituted with 1 to 5 R²⁰ groups,
wherein R⁴ is C₁₋₁₂ alkyl which is optionally substituted with 1 to 5 substituents independently selected from halogen, —OR^{a}, —NR^{a}R^{b},
CN, —C(O)R^{a}, —C(O)OR^{a}, —C(O)NR^{a}R^{b}, —OC(O)NR^{a}R^{b}, —NR^{a}C(O)R^{b}, —NR^{a}C(O)NR^{b}, —NR^{a}C(O)OR^{b}, —SR^{a}, —S(O)₁₋₂R^{a}, —S(O)₂NR^{a}R^{b}, —NR^{a}S(O)R^{b}, C₁₋₆haloalkyl, C₃₋₆cycloalkyl, 3 to 6 membered heterocyclyl wherein the 3 to 6 membered heterocyclyl has 1 to 3 heteroatoms selected from oxygen, nitrogen, and sulfur, C₆₋₁₀ aryl, and 5 to 10 membered heteroaryl wherein the 5 to 10 membered heteroaryl has 1 to 3 heteroatoms selected from oxygen, nitrogen, and sulfur,
wherein each of the C₃₋₆cycloalkyl, 3 to 6 membered heterocyclyl, C₆₋₁₀ aryl, and 5 to 10 membered heteroaryl is optionally substituted with 1 to 5 R²¹ groups,
wherein each R²⁰ is independently selected from the group consisting of halogen, C₁₋₆haloalkyl, CN, —NR^{a}R^{b}, S(O)₁₋₂R^{a}, and OR^{a},
wherein each R²¹ is independently selected from the group consisting of halogen, C₁₋₆alkyl, C₁₋₆haloalkyl, CN, —NR^{a}R^{b}, S(O)₁₋₂R^{a}, and OR^{a}, and
wherein each R^{a} and R^{b} are independently selected from the group consisting of hydrogen and C₁₋₆alkyl, wherein each of the C₁₋₆alkyl is optionally substituted with 1 to 5 substituents independently selected from halogen, hydroxyl, amino, 5 to 10 membered heteroaryl wherein the 5 to 10 membered heteroaryl has 1 to 3 heteroatoms selected from oxygen, nitrogen, and sulfur, and C₁₋₆haloalkyl,
provided that when R¹ is Cl, R² is H and R³ is H then R⁴ is not CH₂CH₂OMe or CH₂CH₂SO₂Me.

8.-9. (canceled)

10. The therapeutic combination of claim 6, wherein the first non-naturally occurring nucleic acid molecule further comprises a polynucleotide sequence encoding a signal sequence operably linked to the N-terminus of the truncated HBV core antigen, and the second non-naturally occurring nucleic acid molecule further comprises a polynucleotide sequence encoding a signal sequence operably linked to the N-terminus of the HBV polymerase antigen.

11. The therapeutic combination of claim 1, wherein
a) the truncated HBV core antigen consists of the amino acid sequence of SEQ ID NO: 2 or SEQ ID NO: 4; and
b) the HBV polymerase antigen comprises the amino acid sequence of SEQ ID NO: 7.

12. The therapeutic combination of claim 1, wherein each of the first, and second non-naturally occurring nucleic acid molecules is a DNA molecule.

13. The therapeutic combination of claim 6, comprising the first non-naturally occurring nucleic acid molecule and the second non-naturally occurring nucleic acid molecule in the same non-naturally nucleic acid molecule.

14. The therapeutic combination of claim 6, comprising the first non-naturally occurring nucleic acid molecule and the second non-naturally occurring nucleic acid molecule in two different non-naturally occurring nucleic acid molecules.

15. The therapeutic combination of claim 6, wherein the first polynucleotide sequence comprises a polynucleotide sequence having at least 90% sequence identity to SEQ ID NO: 1 or SEQ ID NO: 3.

16. The therapeutic combination of claim 15, wherein the first polynucleotide sequence comprises the polynucleotide sequence of SEQ ID NO: 1 or SEQ ID NO: 3.

17. The therapeutic combination of claim 6, wherein the second polynucleotide sequence comprises a polynucleotide sequence having at least 90% sequence identity to SEQ ID NO: 5 or SEQ ID NO: 6.

18. The therapeutic combination of claim 17, wherein the second polynucleotide sequence comprises the polynucleotide sequence of SEQ ID NO: 5 or SEQ ID NO: 6.

19. The therapeutic combination of claim 1, wherein the compound is selected from the group consisting of
2-amino-8-(1,4-dihydroquinazolin-2-yl)-N,N-dipropyl-3H-1-benzazepine-4-carboxamide,
2-amino-8-(1,4-dihydropyrido[3,4-d]pyrimidin-2-yl)-N,N-dipropyl-3H-1-benzazepine-4-carboxamide,
2-amino-N-(cyclopropylmethyl)-8-(1,4-dihydroquinazolin-2-yl)-N-propyl-3H-1-benzazepine-4-carboxamide,
2-amino-8-(1,4-dihydroquinazolin-2-yl)-N-isobutyl-N-propyl-3H-1-benzazepine-4-carboxamide,
2-amino-8-(5-chloro-1,4-dihydroquinazolin-2-yl)-N,N-dipropyl-3H-1-benzazepine-4-carboxamide,
2-amino-8-(7-chloro-1,4-dihydroquinazolin-2-yl)-N,N-dipropyl-3H-1-benzazepine-4-carboxamide,
2-amino-8-(4,4-dimethyl-1H-quinazolin-2-yl)-N,N-dipropyl-3H-1-benzazepine-4-carboxamide,
2-amino-8-(6-chloro-1,4-dihydroquinazolin-2-yl)-iV,iV-dipropyl-3H-1-benzazepine-4-carboxamide,
2-amino-8-(5-methyl-1,4-dihydroquinazolin-2-yl)-N,N-dipropyl-3H-1-benzazepine-4-carboxamide,
2-amino-8-(5-fluoro-1,4-dihydroquinazolin-2-yl)-N,N-dipropyl-3H-1-benzazepine-4-carboxamide, and
2-amino-8-(6-methoxy-1,4-dihydroquinazolin-2-yl)-N,N-dipropyl-3H-1-benzazepine-4-carboxamide,
or a pharmaceutically acceptable salt thereof.

20. The therapeutic combination of claim 1, wherein the compound is selected from the group consisting of 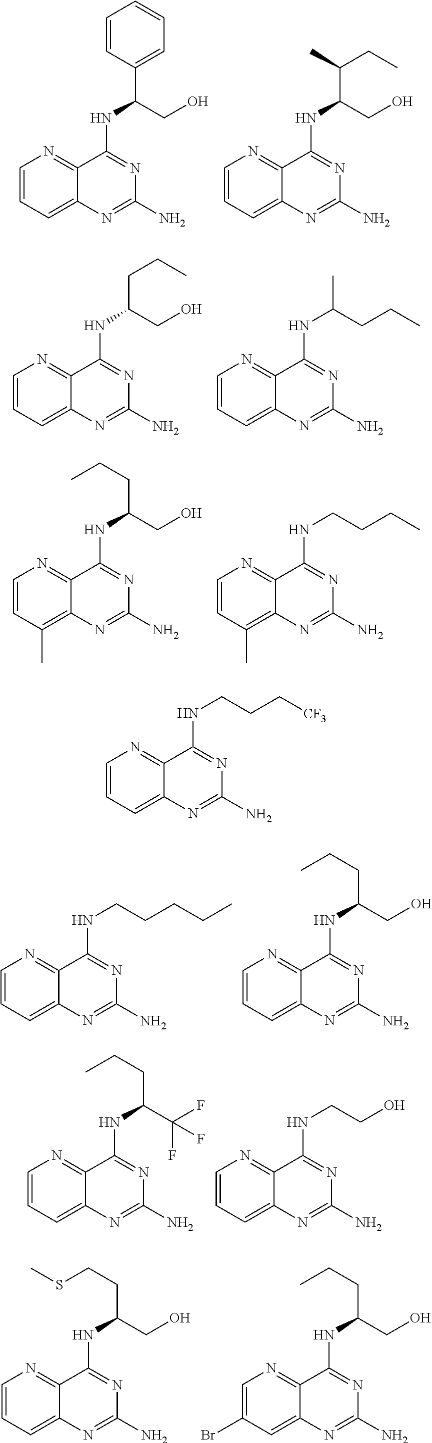 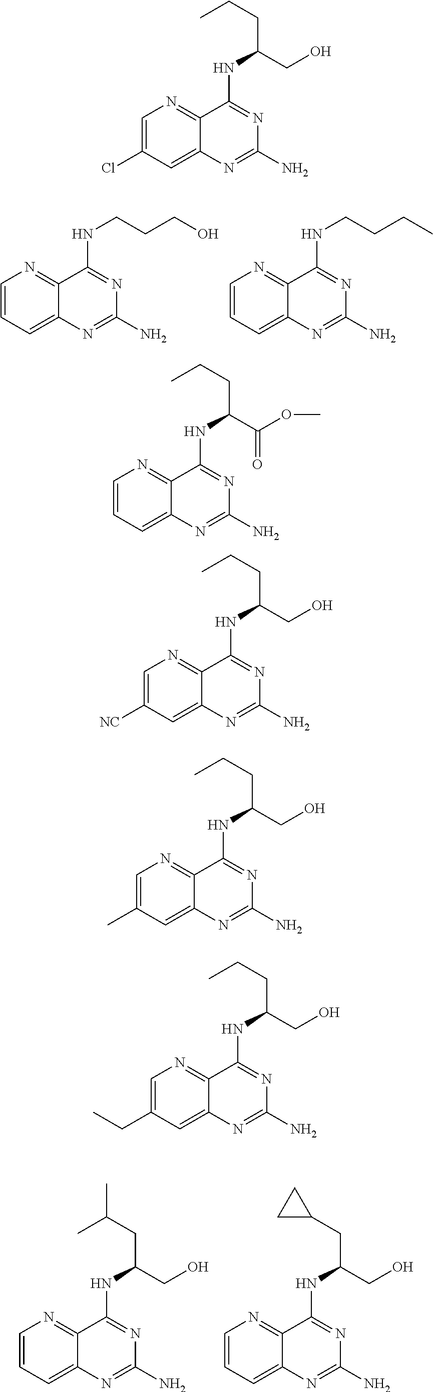 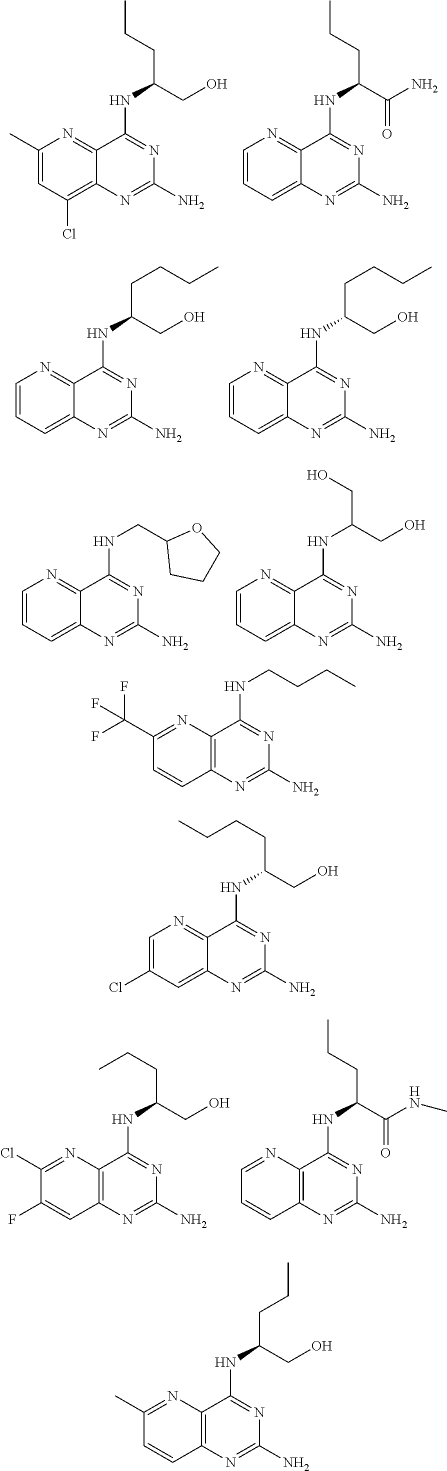 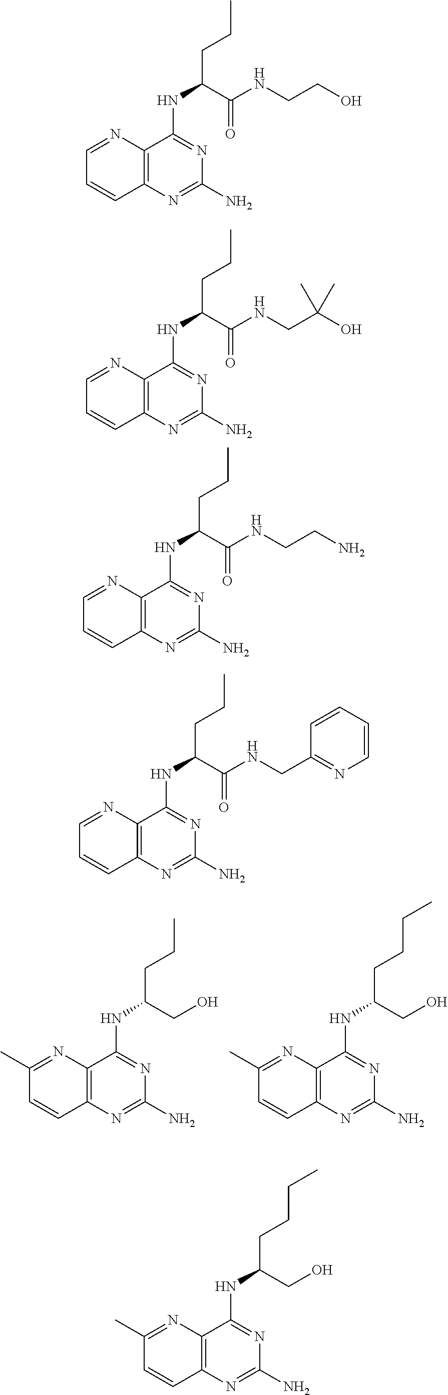 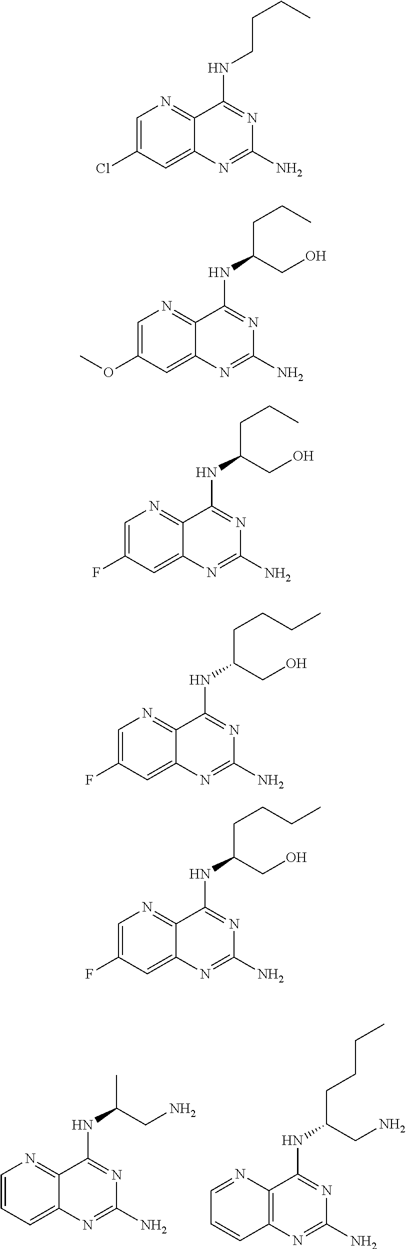 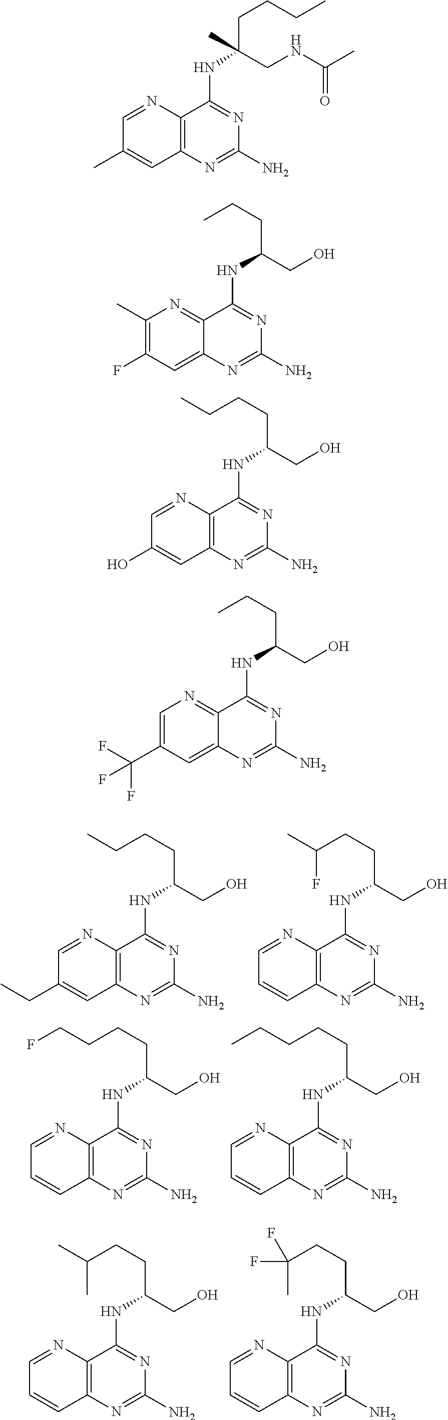 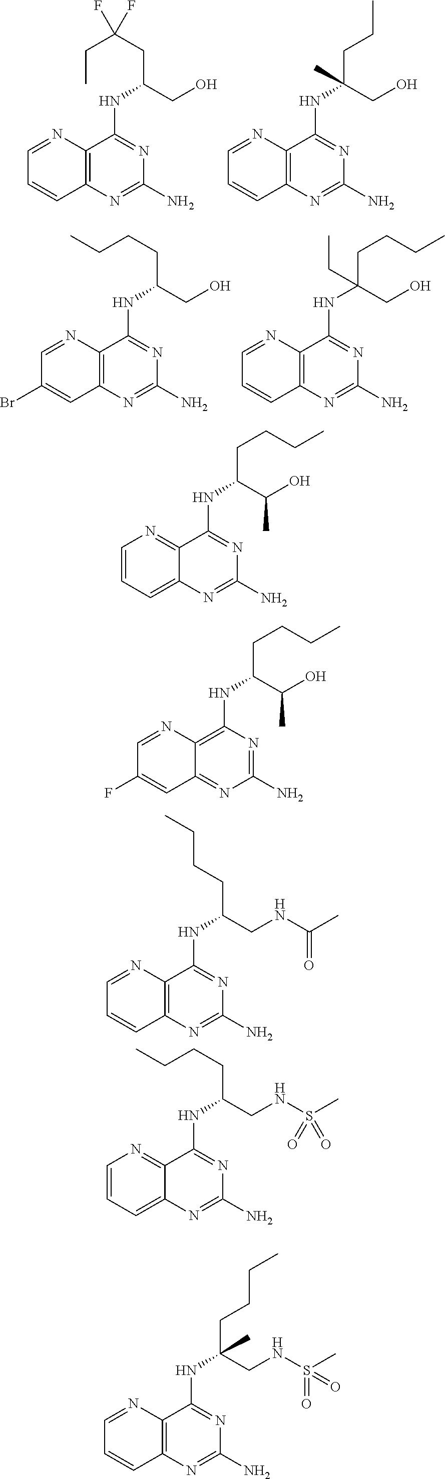 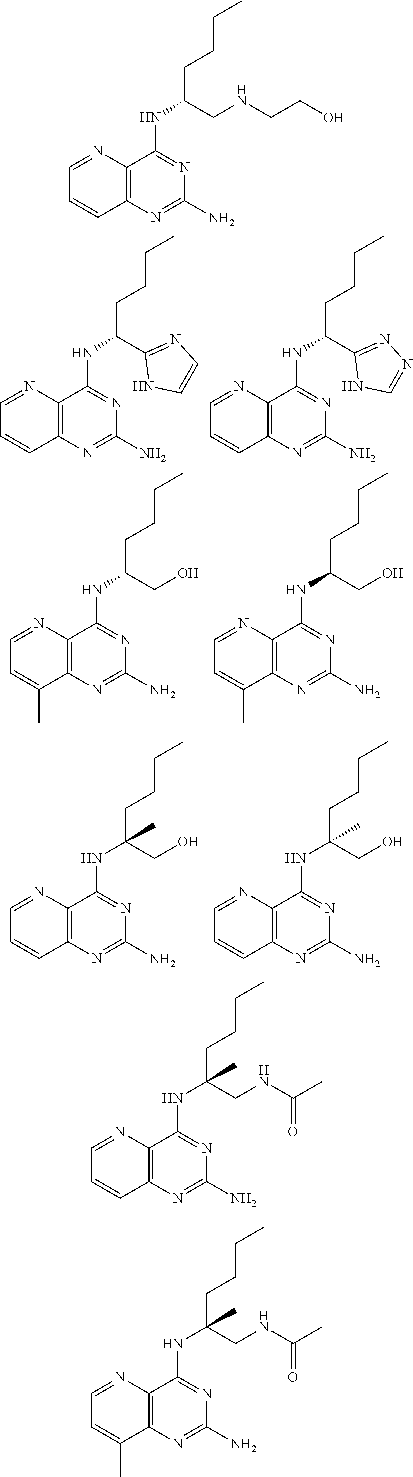 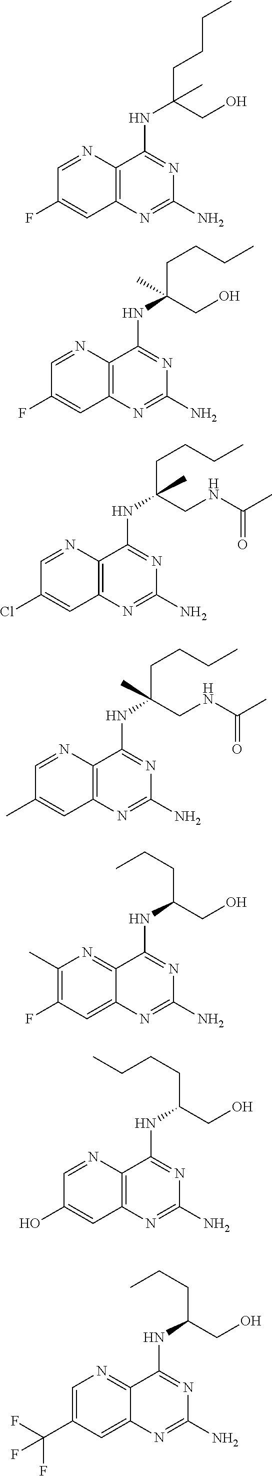 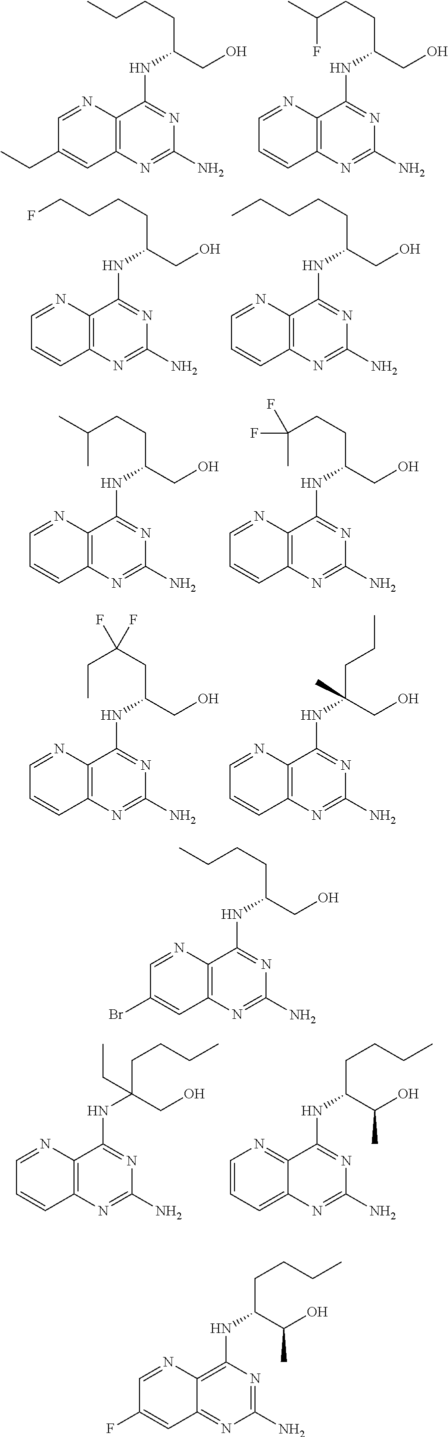 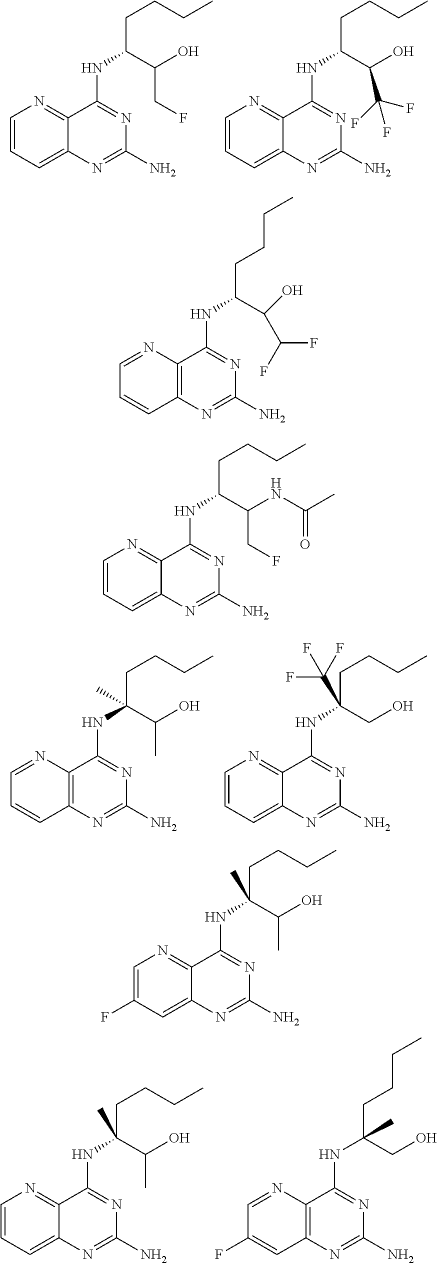 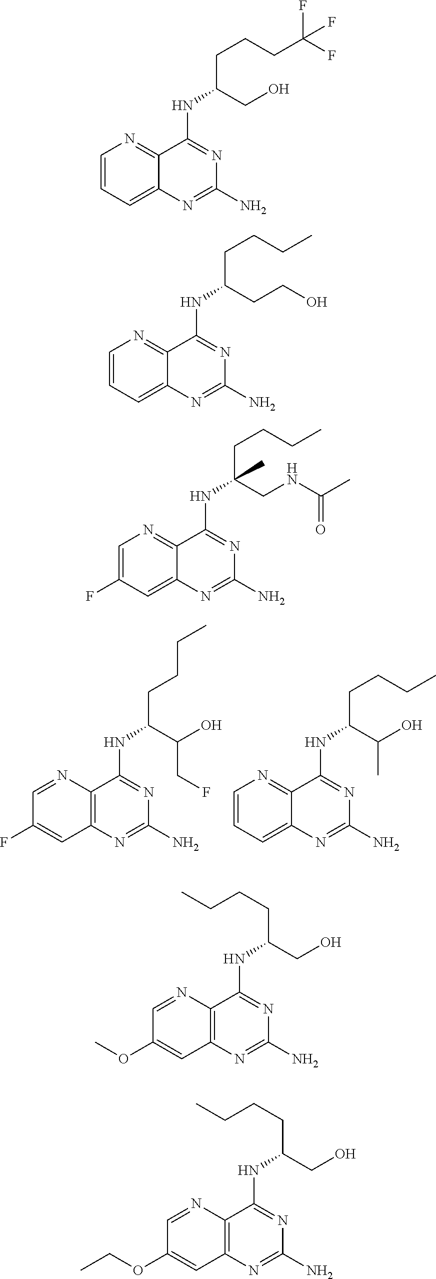 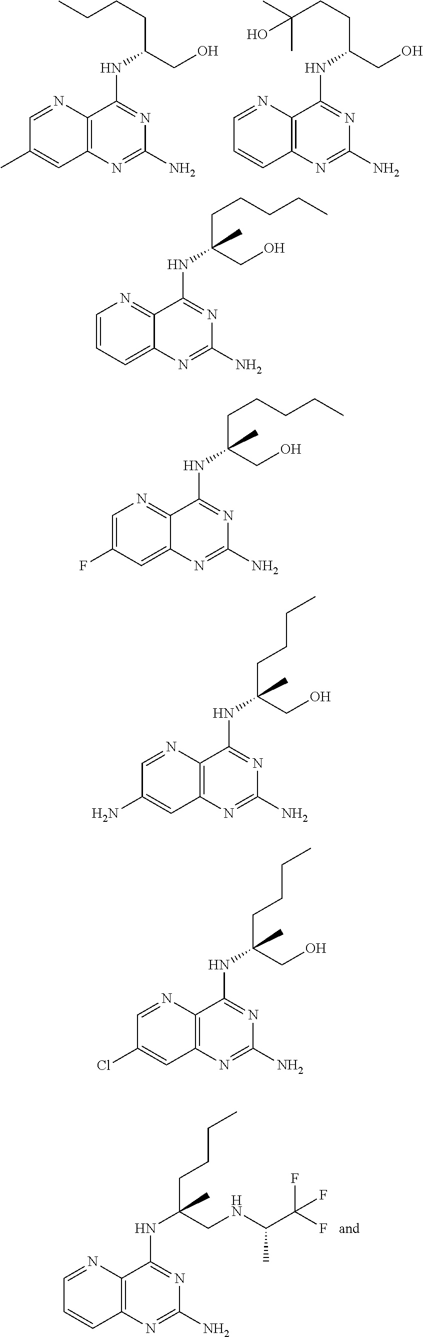 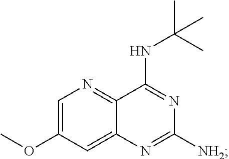 [SMILES: COc1cnc2c(NC(C)(C)C)nc(N)nc2c1]
or a pharmaceutically acceptable salt thereof.

21. The therapeutic combination of claim 1, wherein the compound is selected from the group consisting of 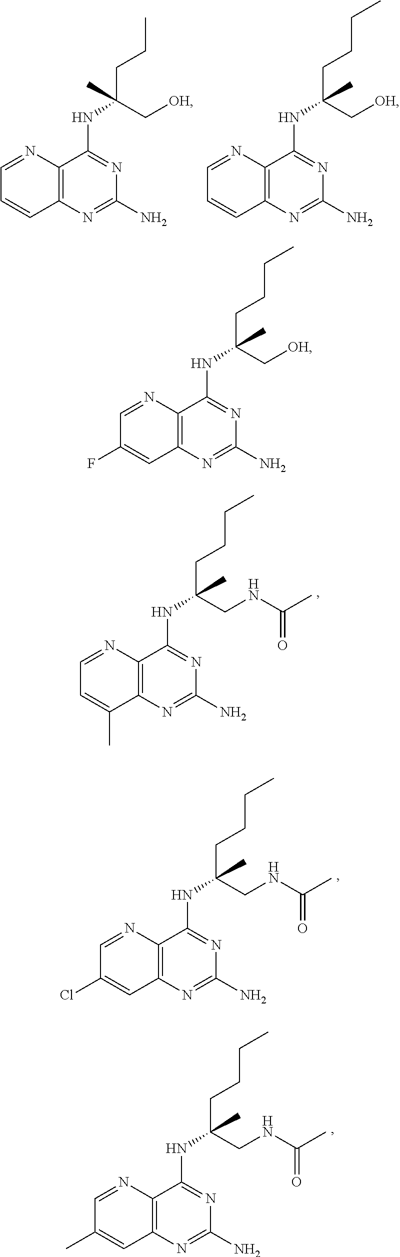 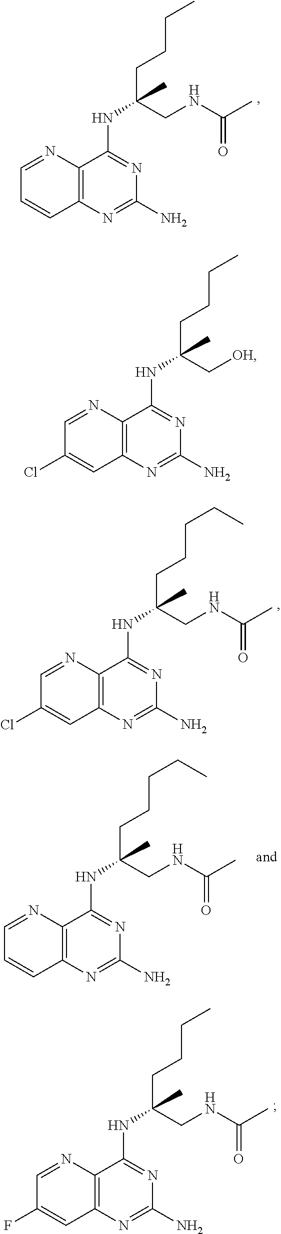
or a pharmaceutically acceptable salt thereof.

22. A kit comprising the therapeutic combination of claim 1, and instructions for using the therapeutic combination in treating a hepatitis B virus (HBV) infection in a subject in need thereof.

23. A method of treating a hepatitis B virus (HBV) infection in a subject in need thereof, comprising administering to the subject the therapeutic combination of claim 1.
